(19) United States
(12) Patent Application Publication
(10) Pub. No.: US 2011/0286916 A1
(43) Pub. Date: Nov. 24, 2011

(54) GENERATION AND CHARACTERIZATION OF ANTI-NOTCH ANTIBODIES FOR THERAPEUTIC AND DIAGNOSTIC USE

(76) Inventors: Jose Miguel Aste-Amezaga, Harleysville, PA (US); Ningyan Zhang, Ambler, PA (US); Fubao Wang, Dresher, PA (US); Andrew Bett, Lansdale, PA (US); Anna Demartis, Rome (IT); Maurizio Nuzzo, Rome (IT); Paolo Monaci, Rome (IT); Stephen Blacklow, Cambridge, MA (US); Jon Aster, Lexington, MA (US)

(21) Appl. No.: 13/130,441

(22) PCT Filed: Nov. 16, 2009
(86) PCT No.: PCT/US09/64520

§ 371 Date: Aug. 3, 2011

(30) Foreign Application Priority Data
Nov. 20, 2008 (US) 61/199753

(51) Int. Cl. A61K 39/395; C12N 1/20; A61P 35/00; A61K 49/00; A61K 51/10; C12N 9/96; C07K 16/28; G01N 33/53

(52) U.S. Cl. 424/1.49; 530/389.1; 530/387.3; 435/252.1; 435/252.8; 435/252.5; 435/253.3; 435/7.92; 435/7.23; 424/9.1; 424/172.1; 424/178.1; 530/391.7; 530/391.3; 435/188

(57) ABSTRACT

The present invention relates to mammalian antibodies, preferably fully human monoclonal antibodies and antigen-binding portions thereof that specifically bind to a cell surface receptor, wherein the receptor protein is a Notch1 receptor protein. Some of the disclosed antibodies bind Notch1 to the exclusion of other members of the Notch receptor family, while other antibodies bind Notch 1 and Notch3. Nucleic acid molecules encoding the Notch antibodies as well as methods of use thereof are also disclosed. Also included are pharmaceutical compositions comprising these antibodies and methods of using the antibodies and compositions thereof for treatment and diagnosis of pathological hyperproliferative oncogenic disorders associated with expression of Notch1 or Notch3 including aberrant activation of each of these receptors.

### FIELD OF THE INVENTION

The invention relates to prevention and treatment of diseases and conditions associated with aberrant Notch activity. This is accomplished through use of agonists or antagonists of Notch. A preferred agonist/antagonist is a monoclonal antibody having specificity for human Notch1 or Notch1/Notch3.

### BACKGROUND OF THE INVENTION

Intrinsic, cell-autonomous factors as well as non-autonomous, short-range and long-range signals guide cells through distinct developmental paths. An organism frequently uses the same signaling pathway within different cellular contexts to achieve unique developmental goals. Notch signaling is an evolutionarily conserved mechanism used to control cell fates through local cell interactions. Signals transmitted through the Notch receptor, in combination with other cellular factors, influence differentiation, proliferation and apoptotic events at all stages of development (Artavanis-Tsakonas et al., 1999, Science, 284, 770-776).

Notch proteins define a unique class of highly conserved transmembrane receptors regulating cell growth, differentiation, and death in different tissues of multicellular organisms (Artavanis-Tsakonas et al., 1999, Science 284, 770-776). Four mammalian Notch homologs have been identified and are designated Notch1, Notch2, Notch3 and Notch4. Human Notch1 (also known as Notch gene homolog 1 and TAN-1) was first identified in 1991 and later mapped to chromosome 9q34 through its involvement in chromosomal translocations found in human T-cell Acute Lymphoblastic Leukemias/Lymphomas (T-ALL) (Ellisen et al., 1991, Cell 66, 649-661).

Mature Notch proteins are heterodimeric receptors derived from the cleavage of Notch pre-proteins into an extracellular subunit containing multiple EGF-like repeats and a transmembrane subunit including the intracellular region (Blaumueller et al., Cell, 1997, 90, 281-291). Notch receptors are first synthesized as 300-350 kDa type I single-pass transmembrane glycoproteins. During maturation, Notch precursor polypeptides are proteolytically processed by a furin-like convertase at a site called S1. The resulting two associated subunits, here termed extracellular Notch (N^{EC}) and transmembrane Notch (N^{TM}), constitute the mature heterodimeric form of the protein present at the cell surface (Sanchez-Irizarry et al., 2004, Mol. Cell. Biol. 24, 9265-9273). The N^{EC} subunit contains EGF like repeats that include the region responsible for ligand binding. The EGF repeats are followed by a negative regulatory region (NRR) that participates in restraining premature activation of Notch receptors (Rand et al., 2000, Mol. Cell. Biol. 20, 1825-1835). The NRR includes three Notch-specific Lin12-Notch repeats (LNRs) and the heterodimerization domain (HD), which contains an additional 103-residue sequence up to the furin cleavage site (S1) as well as the extracellular portion of the N^{TM} subunit. The transmembrane segment of N^{TM} is followed by the intracellular region (ICN) that consists of a RAM domain, seven ankyrin repeats (ANK), a transactivation domain (TAD) and a C-terminal PEST region.

Notch activation results following the binding of ligand to the EGF repeats in the extracellular domain of Notch (Artavanis-Tsakonas et al., Science, 1995, 268, 225-232). Several Notch ligands have been identified in vertebrates, including Delta, Serrate and Jagged. The Notch ligands are also transmembrane proteins, having highly conserved structures. Upon binding of the ligands the N^{TM} subunit gets cleaved by a metalloprotease at S2, a site just external to the transmembrane domain (Mumm et al., 2000, Mol. Cell. 5, 197-206). This cleaved product (N^{TM}*) is subsequently proteolyzed again by an enzyme complex called gamma-secretase, with the final intramembrane cleavage occurring just internal to the inner membrane leaflet. Gamma-secretase cleavage results in release of the ICN. Upon its release, ICN travels to the nucleus and interacts with members of the CSL family of transcription factors, mediated through binding of the RAM and ANK fragments (Nam et al., 2003, J. Biol. Chem. 278, 21232-21239). Binding of ICN to CSL results in recruitment of proteins of the Mastermind family, and this ternary complex engages proteins like p300 and other factors to activate transcription of a variety of target genes. However, in the case of Notch1, the complex is short lived and is targeted for degradation by a C-terminal destruction box in its C-terminal PEST region (Aster, 2005, Int. J. Hematol. 82, 295-301).

In the context of experimental cancer immunotherapy, the Notch signaling network is acquiring increasing importance for its possible roles in neoplastic cells and the immune system (Jang et al., Curr. Opin. Mol. Ther., 2000, 2, 55-65). Larsson et al. predicted that the human Notch genes are proto-oncogenes and candidates for sites of chromosome breakage in neoplasia-associated translocations (Larsson et al., Genomics, 1994, 24, 253-258). The clearest example of oncogenic Notch signaling is found in T-acute lymphoblastic leukemia/lymphoma (T-ALL), an aggressive neoplasm of immature T-cells. Notch1 was identified through its involvement in a (7;9) chromosomal translocation found in approximately 1% of all T-ALLs (Ellisen et al., 1991, Cell 66:649-661). A much broader role was revealed in 2004, with the discovery of two types of activating mutations within Notch1, at least one of which is found in approximately 55-60% of human T-ALLs (Weng et al., 2004, Science 306:269-71). The first type involves the heterodimerization domain and causes changes in amino acid sequence that lead to ligand-independent metalloprotease cleavage at site S2 (Malecki et al., 2006, Mol. Cell. Biol. 26:4642-4651). The second consists of stop codon or frame shift mutations that result in deletion of the C-terminal PEST degron domain and stabilization of ICN1. These remove several possible phosphorylation sites and a conserved WSSSSP sequence that influences the phosphorylation, stability and leukemogenicity of Notch1 in murine models (Chiang et al., 2006, Mol. Cell. Biol. 26:6261-6271).

Synergistic heterodimerization domain and PEST domain mutations are found together in cis in 10-20% of human T-ALL, and low copy number amplifications of Notch1 in T-ALL has also been reported (van Vlierberghe et al., 2006, Leukemia 20:1245-1253). Notch1 mutations are found in combination with all other major known genetic aberrations associated with human T-ALL, and acquired secondary gain-of-function mutations in murine Notch1 have been identified in T-ALLs arising in many different genetic backgrounds (Lin et al., 2006, Blood 107:2540-2543). Transgenes encoding activated forms of Notch3 also cause T-ALL in mice (Bellavia et al., 2000, EMBO J 19:3337-3348). Similar to Notch1, these tumors are dependent on expression of a functional pre-T cell receptor (Bellavia et al., 2002, Proc Natl Acad Sci 99:3788-3793). Taken together, these data indicate that there is continued selection for increasing Notch signaling during T-ALL induction and progression, and that such mutations can be either initiating events or collaborating secondary events.

Notch3 gene amplification and pathway activation have also been reported in ovarian serous carcinoma. Cancer Res., 68:5716-23 (2008). Indeed copy number gain in Notch3 at chromosome 19p13.12 has been reported to occur in ˜20% of high-grade serous carcinomas, and overexpression of Notch3 was observed in nearly 50% of the cases examined, suggesting the significant role that Notch3 signaling contributes to tumor progression in ovarian cancer. Park et al., “Notch-3 gene amplification in ovarian cancer.” Cancer Res., 66:6312-8r (2006).

Beyond T-ALL, there is increasing evidence that Notch signals are oncogenic in other cellular contexts, particularly breast epithelium and melanocytes. To date, cell autonomous gain-of-function mutations in Notch receptors have not been found in human solid tumors, suggesting that ligand-mediated activation predominates in these contexts. The expression of Notch receptors is increased in the human breast and the expression of ligands such as Jagged1 correlates with a more aggressive disease course in breast cancer (Reedijk et al., 2005, Cancer Res. 65:8530-8537). Recent reports indicate that Notch1 signaling is increased in a variety of molecularly different types of breast cancers, and that such cancers are reverted to a more benign phenotype by enforced expression of Numb (Stylianou et al., 2006, Cancer Res. 66:1517-1525). There also appears to be an important positive oncogenic collaboration in breast cancer cells between Notch and Wnt, another pathway that was initially implicated in breast cancer through the analysis of retroviral insertions in murine tumors. Other work in breast cancer cell lines suggests that Notch1 inhibits p53-mediated apoptosis by stimulating signaling through the PI3K-Akt-mTOR-eIF4E pathway (Mungamuri et al., 2006, Cancer Res. 66:4715-4724) and antagonizes the growth suppressive effects of the TGF-β signaling pathway, possibly through the interaction of activated Notch1 with Smad3 (Sun et al., 2005, Oncogene 24:5365-5374).

The expression of Notch receptors and their downstream target genes is also upregulated in primary human melanomas, and enforced expression of constitutively active Notch1 promotes melanoma progression (Balint et al., 2005, J. Clin. Invest. 115:3166-3176). These oncogenic effects correlate with the activation of Wnt signaling in melanoma cells, which promotes the expression of adhesion molecules such as N-cadherin through the transcription factor TCF/LEF. Conversely, previous work suggests that Notch1 activation transmits anti-oncogenic signals in prostate cancer cells. Experiments performed using a murine model of prostate tumor formation suggests that Notch1 signals can inhibit prostate cancer progression. In addition, enforced expression of ICN1 inhibits the growth of various prostate cancer cell lines (Shou et al., 2001, Cancer Res. 61:7291-7297).

Disclosed and claimed in U.S. Pat. No. 5,789,195 are nucleic acid sequences encoding Notch genes. Antibodies to human Notch proteins are additionally provided (Artavanis-Tsakonas et al., 1998). Amino acid sequences of Notch genes and antibodies against Notch proteins are also disclosed and claimed in U.S. Pat. No. 6,090,922 (Artavanis-Tsakonas et al., 2000).

Disclosed in U.S. Pat. No. 6,083,904 and PCT publication WO 94/07474 are therapeutic and diagnostic methods and compositions based on Notch proteins and nucleic acids, wherein antisense methods are generally disclosed (Artavanis-Tsakonas, 2000; Artavanis-Tsakonas et al., 1994).

Currently, there are no known therapeutic agents that effectively modulate the activation of Notch receptors. Modulation of expression of Notch genes may prove to be a useful point for therapeutic intervention in developmental, hyperproliferative or autoimmune disorders or disorders arising from aberrant apoptosis.

In view of the importance of the Notch signaling pathway and its role in human cell differentiation and disease, there is a need in the art for identification of Notch modulators and for methods and therapeutic agents for intervening in diseases and conditions related to defects in the Notch pathway. Towards this end, the present invention provides antibodies specific for Notch1 or Notch1/Notch3 that are effective at preventing the Notch activated signaling cascade that has been implicated in various hyper-proliferative disorders. Compositions and methods for modulating expression of Notch genes, including expression of variants of Notch are also provided.

### SUMMARY OF THE INVENTION

Embodiments of this invention are made available by the development of antibodies that retain favorable affinity to the Notch receptor protein, particularly human Notch1 receptor protein. The antibodies described infra, (“Invention Antibodies”) offer an important new approach to the treatment of various disorders of cell fate, in particular hyperproliferative disorders (e.g., cancer). Disorders involving aberrant Notch receptor activation or undesirable levels of expression or activity of Notch1 protein are also included.

A broad aspect of the invention provides a plurality of anti-Notch antibodies, preferably anti-Notch1 monoclonal antibodies as well as those that bind Notch1 and Notch3 (Notch1/3). The monoclonal antibodies of the invention bind to the human Notch 1 receptor (Notch1) and can thus be useful in methods to treat or diagnose pathological hyperproliferative oncogenic disorders mediated by Notch1 expression or dysplastic cells associated with increased expression or activity of the Notch1 receptor protein.

An embodiment of this invention relates to the antibodies described herein, including the sequences of the VRs, FRs and CDRs polypeptides and the polynucleotides encoding them. Variant antibodies exemplified by diabody, bi-specific, trivalent & tetravalent antibodies or other antibodies derived from the herein described invention antibodies are also encompassed by the invention.

The invention particularly relates to at least one monoclonal antibody, or binding fragment thereof described herein that binds specifically to an antigen present in various cancers including but not limited to T cell acute Lymphoblastic leukemia (T-ALL) human breast cancer, human colon cancer, melanoma, human lung cancer and human prostate cancer, wherein the antigen is Notch1 (i) a polypeptide having a molecular weight of about 300 kDa as determined by SDS-PAGE under reducing conditions, which, upon activation, releases its cytoplasmic domain that travels to the nucleus. In another embodiment, some of the invention antibodies bind Notch1 and Notch3.

The invention also provides an isolated cell line that produces at least one anti-Notch1 antibody as described herein. An embodiment of the invention thus provides an isolated cell line which produces at least one or more of the monoclonal antibodies as detailed herein that binds specifically an antigen present in one of T cell acute Lymphoblastic leukemia (T-ALL), human breast cancer, human colon cancer, melanoma, human lung cancer and human prostate cancer, the antigen being Notch1 receptor protein (i) a polypeptide having a molecular weight of about 300 kDa as determined by SDS-PAGE under reducing conditions.

In certain embodiments, at least one invention described herein binds to the ligand binding domain of the Notch1 receptor.

In yet another embodiment, at least one antibody of the invention binds to the negative regulator region, resident in the extracellular domain of the Notch1 receptor.

In still another embodiment, at least one antibody of the invention is specific for a Notch1 receptor characterized by putative tumor-associated mutations associated with Notch signaling and HD domain stability.

It is understood that the term “antibody” includes “antibodies” such as one or more of the Notch1 specific antibodies described herein including those that also bind Notch3. As well, it includes monoclonal, polyclonal, multivalent, bispecific, and trivalent or optimized antibodies including fragments thereof. The invention also contemplates the use of single chains such as the variable heavy and light chains of the antibodies. Generation of any of these types of antibodies or antibody fragments is well known to those skilled in the art. In the present case, monoclonal antibodies to Notch1 receptor proteins have been generated and have been isolated and shown to have high affinity to Notch1. While the remaining discussion details Notch1 specific antibodies of the invention, the same discussion applies equally to those invention antibodies that bind Notch1 and Notch3.

The invention also includes modifications to the invention antibodies including variants thereof which do not significantly affect their binding properties. Such variants may have enhanced or decreased activity towards its binding partner.

Another embodiment of the invention encompasses monoclonal antibody or binding fragment thereof that may be Fab fragments, F(ab)2 fragments, Fab′ fragments, F(ab′)2 fragments, Fd fragments, Fd′ fragments or Fv fragments, Fv, scFv, scFv-Fc or diabodies or any functional fragment whose half-life would have been increased by a chemical modification, especially by PEGylation, or by incorporation in a liposome. It may also be an anti-idiotypic antibody. Plasma protein binding can be an effective means of improving the pharmacokinetic properties of otherwise short lived molecules.

One general strategy of reducing a therapeutic proteins intrinsic rate of clearance is via amino acid substitutions. In case of a protein, this strategy may entail amino acid substitutions that reduce receptor binding affinity in intracellular endosomal compartments, thereby leading to increased recycling in the ligand-sorting process and consequently resulting in longer half-life in extracellular medium. See Sarkar C. A., Lowenhaupt K., Horan T., Boone T. C., Tidor B., Lauffenburger D. A. Nat. Biotechnol. (2002) 20:908-913. A second approach is to express the therapeutic protein as a genetic fusion with a natural protein that has a long serum half-life; either 67 kDa serum albumin (SA)—Syed S., Schuyler P. D., Kulczycky M., Sheffield W. P. Blood (1997) 89:3243-3252) or the Fc portion of an antibody, which adds an additional 60-70 kDa in its natural dimeric form, depending on glycosylation (Mohler et al., J. Immunol., 151:1548-1561 (1993). As a consequence, an embodiment of the invention provides modifications to at least one antibody disclosed herein resulting in a fusion protein comprising an antibody of the invention fused to albumin. See Dennis et al., “Albumin binding as a general strategy for improving the pharmacokinetics of proteins.” J Biol Chem., 277:35035-43 (2002).

Glycosylated variants (Glycoforms) of the invention antibodies are also envisioned. In one embodiment of the invention, antibodies, or fragments thereof, are modified to reduce or eliminate potential glycosylation sites. The amino acids where carbohydrate, such as oligosaccharide, is attached are typically asparagine (N-linkage), serine (O-linkage), and threonine (O-linkage) residues. In order to identify potential glycosylation sites within an antibody or antigen-binding fragment, the sequence of the antibody is examined, for example, by using publicly available databases such as the website provided by the Center for Biological Sequence Analysis (see http://www.cbs.dtu.dk/services/NetNGlyc/ for predicting N-linked glycosylation sites) and http://www.cbs.dtu.dk/services/NetOGlyc/ for predicting O-linked glycosylation sites). Additional methods for altering glycosylation sites of antibodies are described in U.S. Pat. Nos. 6,350,861 and 5,714,350, the entire content of each of which is incorporated herein in its entirety. In order to improve the binding affinity of an antibody or antigen-binding fragment thereof, glycosylation sites of the antibody can be altered, for example, by mutagenesis (e.g., site-directed mutagenesis). Such modified antibodies having reduced glycosylation sites or carbohydrates relative to the unmodified form are referred to as “aglycosylated” antibodies. An “afucosylated” anti-Notch1 antibody derived from one or more antibodies described herein is representative of such an antibody that falls within the scope of the invention. See Li et al., Nat. Biotechnol., 24: 210-215 (2006); Shields, R. L. et al. Lack of fucose on human IgG1 N-linked oligosaccharide improves binding to human FcγRIII and antibody-dependent cellular toxicity. J. Biol. Chem. 277, 26733-26740 (2002). In alternative embodiment, the invention antibodies or antigen binding fragments thereof are modified to enhance glycosylation.

Fc engineered variants of the invention antibodies are also encompassed by the present invention. Such variants include antibodies or antigen binding fragments thereof which have been engineered so as to introduce mutations or substitutions in the Fc region of the antibody molecule so as to improve or modulate the effector functions of the underlying antibody molecule relative to the unmodified antibody. In general, improved effector functions is included to refer to such activities as CDC and ADCC. Further thereto, the invention provides Fc variants that have improved function and/or solution properties as compared to the aglycosylated form of the parent Fc polypeptide. Improved functionality herein includes but is not limited to binding affinity to an Fc ligand. Improved solution properties herein includes but is not limited to stability and solubility. In one embodiment, the proposed Fc variants bind to an FcγR with an affinity that is within about 0.5-fold of the glycosylated form of the parent Fc polypeptide. In an alternate embodiment, the aglycosylated Fc variants bind to an FcγR with an affinity that is comparable to the glycosylated parent Fc polypeptide. In an alternate embodiment, the Fc variants bind to an FcγR with an affinity that is greater than the glycosylated form of the parent Fc polypeptide.

Another broad aspect of the invention comprises an antibody or a binding fragment thereof that comprises a light chain comprising at least one complementarity determining region CDR having an amino acid sequence as set forth in one or more appendices detailed herein (see appendices III, IV and IV) or at least one CDR whose sequence has at least 80%, preferably 85%, 90%, 95% and 98% identity, after optimum alignment, with the sequences set forth in one or more appendices described herein or a heavy chain comprising at least one CDR comprising an amino acid sequence selected from the group set forth in one or more appendices set forth herein or at least one CDR whose sequence has at least 80%, preferably 85%, 90%, 95% and 98% identity, after optimum alignment, with said at least one CDR as set forth in one or more appendices set forth herein. Alternatively, the antibody of the invention comprises at least one heavy chain and/or a light chain comprising at least one amino acid sequence as set forth in one of Appendix III, IV or IX. Nucleic acid molecules comprising nucleotide sequences encoding at least one or more of the above referenced amino acid sequences are also contemplated.

The light chain may likewise comprise the amino acid sequence as set forth in one or more appendices detailed herein, while the heavy chain may comprise the amino acid sequence as set forth in one or more appendices set forth herein. See appendix III, IV or IX.

Antibodies that compete with any one or more of the antibodies described herein for binding with Notch 1 are also within the scope of the invention.

In recent years, various strategies have been developed for preparing scFv as a multimeric derivative. This is intended to lead, in particular, to recombinant antibodies with improved pharmacokinetic and biodistribution properties as well as with increased binding avidity. In order to achieve multimerisation of the scFv, scFv may be prepared as fusion proteins with multimerisation domains. The multimerisation domains may be, e.g. the CH3 region of an IgG or coiled coil structure (helix structures) such as Leucin-zipper domains. However, there are also strategies in which the interaction between the VH/VL regions of the scFv are used for the multimerisation (e.g. di-, tri- and pentabodies).

Also considered are multivalent antibody constructs that are Notch 1 antagonists or agonists. In one embodiment, a multivalent antibody construct comprises at least one antigen recognition site specific for Notch1 receptor protein. In certain embodiments, at least one of the antigen recognition sites is located on a scFv domain, while in other embodiments, all antigen recognition sites are located on scFv domains.

Also contemplated herein is a multivalent, multispecific antibody or fragment thereof comprising more than one antigen binding site having an affinity toward a Notch1 target antigen and one or more hapten binding sites having affinity towards hapten molecules. Also preferred, the multivalent, multispecific antibody or fragment thereof further comprises a diagnostic/detection and/or therapeutic agent.

The phrase “multivalent antibody” or “multivalent antibody construct” refers to an antibody or antibody construct comprising more than one antigen recognition site. For example, a “bivalent” antibody construct has two antigen recognition sites, whereas a “tetravalent” antibody construct has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody construct of the invention. For example, a “monospecific” antibody construct's antigen recognition sites all bind the same epitope. A “bispecific” antibody construct has at least one antigen recognition site that binds a first epitope and at least one antigen recognition site that binds a second epitope that is different from the first epitope. A “multivalent monospecific” antibody construct has multiple antigen recognition sites that all bind the same epitope. A “multivalent bispecific” antibody construct has multiple antigen recognition sites, some number of which bind a first epitope and some number of which bind a second epitope that is different from the first epitope.

In still another embodiment, the antibody construct is monospecific. In still another embodiment, the multivalent antibody is tetravalent. In one embodiment of the invention, the antibody is a monospecific tetravalent antibody, wherein the antibody comprises four Notch1 antigen recognition sites. In still another embodiment, the antibody construct is specific for an epitope on Notch1.

In another embodiment of the invention, the antibody construct is bispecific. In one embodiment, the antibody construct has two Notch1-specific antigen recognition sites and two Notch3-specific recognition sites.

In another embodiment of the invention, the antibody construct is a trivalent antibody construct specific for the Notch1 receptor protein. In yet another embodiment, the invention contemplates an antibody construct having two Notch1-specific antigen recognition sites and two Notch3-specific recognition sites.

Other antibody constructs may be multispecific for different epitopes on human Notch1 receptor proteins. In any of the multispecific antibody constructs, at least one antigen recognition site may be located on a scFv domain, and in certain embodiments, all antigen recognition sites are located on scFv domains.

In one aspect, the invention provides an antibody fragment comprising: (i) a first polypeptide comprising a light chain variable domain (and in some embodiments further comprising a light chain constant domain), (ii) a second polypeptide comprising a heavy chain variable domain, a first Fc polypeptide sequence (and in some embodiments further comprising a non-Fc heavy chain constant domain sequence), and (iii) a third polypeptide comprising a second Fc polypeptide sequence. Generally, the second polypeptide is a single polypeptide comprising a heavy chain variable domain, heavy chain constant domain (e.g., all or part of CH1) and the first Fc polypeptide. For example, the first Fc polypeptide sequence is generally linked to the heavy chain constant domain by a peptide bond [i.e., not a non-peptidyl bond]. In one embodiment, the third polypeptide comprises an N-terminally truncated heavy chain which comprises at least a portion of a hinge sequence at its N terminus. In one embodiment, the third polypeptide comprises an N-terminally truncated heavy chain which does not comprise a functional or wild type hinge sequence at its N terminus. In some embodiments, the two Fc polypeptides of an antibody of the invention or a fragment thereof are covalently linked. For example, the two Fc polypeptides may be linked through intermolecular disulfide bonds, for instance through intermolecular disulfide bonds between cysteine residues of the hinge region.

In one aspect, the invention provides a composition comprising a population of immunoglobulins wherein at least (or at least about) 50%, 75%, 85%, 90%, 95% of the immunoglobulins are antibody fragments of the invention. A composition comprising said population of immunoglobulins can be in any of a variety of forms, including but not limited to host cell lysate, cell culture medium, host cell paste, or semi-purified or purified forms thereof. Purification methods are well known in the art, some of which are described herein.

Another embodiment of the invention provides a Notch1-specific diabody antibody. By diabody the skilled person means a bivalent homodimeric scFv derivative (Hu et al., 1996, PNAS 16: 5879-5883). The shortening of the Linker in an scFv molecule to 5-10 amino acids leads to the formation of homodimers in which an inter-chain V_{H}/N_{L}-superimposition takes place. Diabodies may additionally be stabilized by the incorporation of disulphide bridges. Examples of diabody-antibody proteins from the prior art can be found in Perisic et al. (1994, Structure 2: 1217-1226).

By minibody the skilled person means a bivalent, homodimeric scFv derivative. It consists of a fusion protein which contains the CH3 region of an immunoglobulin, preferably IgG, most preferably IgG1 as the dimerisation region which is connected to the scFv via a Hinge region (e.g. also from IgG1) and a Linker region. The disulphide bridges in the Hinge region are mostly formed in higher cells and not in prokaryotes. Preferably the minibody is a Notch1-specific minibody antibody fragment. Examples of minibody-antibody proteins from the prior art can be found in Hu et al. (1996, Cancer Res. 56: 3055-61).

By triabody the skilled person means a: trivalent homotrimeric scFv nderivative (Kortt et al. 1997 Protein Engineering 10: 423-433). ScFv derivatives wherein VH-VL are fused directly without a linker sequence lead to the formation of trimers.

The skilled person will also be familiar with so-called mini antibodies which have a bi-, tri- or tetravalent structure and are derived from scFv. The multimerisation is carried out by di-, tri- or tetrameric coiled coil structures (Pack et al., 1993 Biotechnology II:, 1271-1277; Lovejoy et al. 1993 Science 259: 1288-1293; Pack et al., 1995 J. Mol. Biol. 246: 28-34).

Therefore, an alternative embodiment proposes a Notch1-specific multimerised molecule based on the abovementioned antibody fragments and may be, for example, a triabody, a tetravalent mini antibody or a pentabody.

A related aspect of the invention provides monoclonal antibodies or functional fragments thereof that specifically binds human Notch1 with specified affinities. In certain embodiments, these antibodies bind human Notch1 with an ED₅₀ in the range of about 10 pM to about 500 nM. In certain embodiments, these antibodies bind human Notch1 with an ED₅₀ in the range of about 500 pM to about 300 nM.

The present invention further provides an antibody-recognized surface antigen present on host cell, including but not limited to T-cell acute lymphoblastic leukemia/lymphoma, human colon cancer, melanoma, human lung cancer and human prostate cancer, the antigen being Notch1 (i) a polypeptide having a molecular weight of about 300 kDa as determined by SDS-PAGE under reducing conditions or a biologically equivalent variant or fragment thereof.

Also provided is a monoclonal antibody of the invention or a binding fragment thereof that is bound to a solid matrix.

Antibodies to Notch1 as described herein may also be used in production facilities or laboratories to isolate additional quantities of the proteins, such as by affinity chromatography. For example, the antibodies of the invention may also be utilized to isolate additional amounts of Notch1.

In one aspect, the invention provides isolated, purified or recombinant polypeptides having an amino acid sequence that is at least 90%, 95%, 98% or 99% identical to an amino acid sequence as set forth in one or more appendices herein described. In certain embodiments the application provides an amino acid sequence that is at least 90%, 95%, 98%, 99%, 99.3%, 99.5% or 99.7% identical to the target amino acid sequence herein described.

The present invention further relates to a polynucleotide encoding an antibody of the invention. In accordance therewith, the invention further provides: isolated nucleic acid encoding the inventive antibodies disclosed herein including the heavy and/or light chain or antigen-binding portions thereof. Thus, an aspect of the invention provides isolated nucleic acid molecules selected from the nucleotide sequences described in any one or more of the appendices detailed herein. A related aspect is drawn to (a) a nucleic aid molecule described in any one or more of the appendices detailed herein encoding one or more of the sequence of amino acids as set forth in one or more of the appendices described herein; or (b) the nucleotide sequence that hybridizes to the nucleotide sequence of (a) under moderately stringent conditions, or (c) a nucleic acid molecule comprising a nucleotide sequence that is a degenerate sequence with respect to either (a) or (b) above, or (d) splice variant cDNA sequences thereof or (e) a nucleic acid of at least 18 nucleotides capable of hybridizing under conditions of great stringency with at least one of the CDRs of nucleic acid sequence described in one or more of the appendices described herein or with a sequence having at least 80%, preferably 85%, 90%, 95% and 98%, identity after optimum alignment with the sequence as set forth in one or more of the appendices detailed herein.

A vector comprising the nucleic acid molecule described above, optionally, operably linked to control sequences recognized by a host cell transformed with the vector is also provided as is a host cell transformed with the vector. The cells transformed according to the invention can be used in processes for preparation of recombinant antibody disclosed herein. A variety of host cells can be transformed with the nucleic acid molecules encoding the antibody or a fragment thereof. The host cell can be chosen from prokaryotic or eukaryotic systems, for example bacterial cells but likewise yeast cells or animal cells, in particular mammalian cells. It is likewise possible to use insect cells or plant cells. Methods of producing a recombinant protein are well known.

The invention likewise concerns animals, except man, which comprise at least one cell transformed according to the invention. Thus, non-human transgenic animals that express the heavy and/or light chain or antigen-binding portions thereof of an anti-Notch1 antibody are also provided.

The present invention further provides a pharmaceutical composition comprising the monoclonal antibody, or binding fragment thereof, according to the invention, and a pharmaceutically acceptable carrier, excipient, or diluent. The pharmaceutical composition may further comprise another component, such as an anti-tumor agent or an imaging reagent.

Certain embodiments of the invention relate to the use of the invention antibodies as targeted delivery systems for cytotoxic agents such as chemotherapeutic drugs, peptides or radionuclides, for immunological response promoters such as cytokines, for pro-drugs or for gene therapies. As well, the antibodies described herein may also transport/deliver payloads such as RNAi or shRNA. These payloads may be naked or chemically modified. Immunoliposomes as potential delivery vehicles are also included.

As will be appreciated by one skilled in the art, the antibodies of the invention or binding fragments thereof will also find use in various medical or research purposes, including staging of various pathologies associated with expression of Notch1. Indeed, laboratory research may also be facilitated through use of such antibodies. Identifying patients at risk of a pathological effect of an oncogenic disorder associated with expression of Notch1, particularly hyperproliferative oncogenic disorders such as, but not limited to, T cell acute Lymphoblastic leukemia (T-ALL), human breast cancer, human colon cancer, melanoma, human lung cancer and human prostate cancer is also encompassed. As would be recognized by one of ordinary skill in this art, the level of antibody expression associated with a particular disorder will vary depending on the nature and/or the severity of the pre-existing condition.

As a consequence, additional embodiments of the invention pertain to the use of the invention antibodies for detecting dysplastic or neoplastic Notch1 bearing cells as well as diagnosing, assessing and treating disorders associated with expression of Notch1 receptor protein or aberrant activation of the Notch cascade.

As used herein, the term “an oncogenic disorder associated with expression of Notch1” is intended to include diseases and other disorders in which the presence of high levels or abnormally low levels of Notch1 receptor protein (aberrant) in a subject suffering from the disorder has been shown to be or is suspected of being either responsible for the pathophysiology of the disorder or a factor that contributes to a worsening of the disorder. Thus, “neoplastic cells” or “neoplasia associated with expression of Notch1” or “dysplastic cells associated with expression of Notch1” which are used interchangeably refer to abnormal cells or cell growth characterized by increased or decreased expression levels of Notch1 relative to normal. Such transformed cells proliferate without normal homeostatic growth control resulting in a condition marked by abnormal proliferation of cells of a tissue—cancer. Alternatively, such disorders may be evidenced by an increase in the levels of Notch1 on the cell surface or in increased ICD levels in the affected cells or tissues of a subject suffering from the disorder. The increase in Notch1 levels may be detected, for example, using an anti-Notch1 antibody as described above. More, it refers to cells which exhibit relatively autonomous growth, so that they exhibit an aberrant growth phenotype characterized by a significant loss of control of cell proliferation. Alternatively, the cells may express normal levels of Notch1 but are marked by abnormal proliferation. Not all neoplastic cells are necessarily replicating cells at a given time point. The cells defined as neoplastic cells consists of cells in benign neoplasms and cells in malignant (or frank) neoplasms. Frankly, neoplastic cells are frequently referred to as cancer, typically termed carcinoma if originating from cells of endodermal or ectodermal histological origin, or sarcoma if originating from cell types derived from mesoderm.

In certain embodiments, “increased expression” as it relates to Notch1 refers to protein or gene expression levels that demonstrate a statistically significant increase in expression (as measured by RNA expression or protein expression) relative to a control. As well “increased expression” is also used to encompass “increased activation of the Notch cascade”. Thus, in some disorders associated with expression of Notch1, the level of expression of Notch1 may not be increased relative to a control but the level of activation of the Notch cascade may be increased relative to a control or a patient without the disease.

Administration of the antibodies of the present invention in any of the conventional ways known to one skilled in the art (e.g., topical, parenteral, intramuscular, IV, subcutaneous etc.), will provide an extremely useful method of detecting dysplastic cells in a sample as well as allowing a clinician to monitor the therapeutic regiment of a patient undergoing treatment for a hyperproliferative disorder associated with or mediated by expression of Notch1.

It is known in the art to use antibodies to detect the presence or expression of a specific protein. Because Notch1 may be overexpressed in certain hyperproliferative disorders including, for example, cancer, Notch1-specific antibodies of this invention may be used to detect the overexpression and, thus, to detect metastatic disease. As well, the immunodetection methods of the present invention may be of utility in the diagnosis of various disease states. As well, the invention antibodies may be exploited to detect differential expression of Notch1 in metastatic cells. As will be apparent to the skilled artisan human Notch1 or ICD or any other downstream target may be detected in a number of ways such as by various assays. Immunodetection techniques include but are not limited to immunohistological staining, western blotting, dot blotting, precipitation, agglutination, ELISA assays, immunohistochemistry, in situ hybridization, flow cytometry or radio-immunoassay (RIA) technique or equivalent on a variety of tissues and a variety of sandwich assays. These techniques are well known in the art. See, for example, U.S. Pat. No. 5,876,949, hereby incorporated by reference.

When used with suitable labels or other appropriate detectable biomolecule or chemicals, the antibodies described herein are particularly useful for in vitro and in vivo diagnostic and prognostic applications. Suitable conditions for which the antibody of the invention will find particular use for include the detection and diagnosis of neoplasias, such as, but not limited to cancer of the ovary, prostate, colon and skin. Inflammatory responses or disorders triggered by Notch receptor activation or cascade area also included.

Labels for use in immunoassays are generally known to those skilled in the art and include enzymes, radioisotopes, and fluorescent, luminescent and chromogenic substances, including colored particles such as colloidal gold or latex beads. Various types of labels and methods of conjugating the labels to the antibodies of the invention are well known to those skilled in the art, such as the ones set forth below.

In accordance with the above, an illustrative embodiment provides a method for detecting normal, benign, hyperplastic, and/or cancerous cells or a portion thereof in a biological sample comprising: providing a Notch1 antibody or binding portion thereof which recognizes an antigen (Notch1) on the surface of the cells, wherein the antibody or binding portion thereof binds to an epitope of Notch which is also recognized by any one or more monoclonal antibodies detailed herein and wherein the antibody or binding portion thereof is bound to a label effective to permit detection of the cells or a portion thereof upon binding of the antibody or binding portion thereof to the antigen; contacting the biological sample with the antibody or binding portion thereof having a label under conditions effective to permit binding of the antibody or binding portion thereof to the antigen on any of the cells or a portion thereof in the biological sample; and detecting the presence of any of the cells or a portion thereof in the biological sample by detecting the label.

In certain embodiments, the step of contacting the antibody is carried out in a living mammal and comprises: administering the Notch1 antibody or binding portion thereof to the mammal under conditions effective to permit binding of the antibody or binding portion thereof to the antigen on any of the cells or a portion thereof in the mammal.

In certain embodiments, the invention antibodies may be labeled with a detectable moiety, such as a fluorophore, a chromophore, a radionuclide, a chemiluminescent agent, a bioluminescent agent and an enzyme.

Yet another embodiment of the invention provides a method of diagnosis, preferably in vitro, of illnesses connected with an overexpression or under expression, preferably overexpression of the Notch1 receptor. Samples are taken from the patient and subject to any suitable immunoassay with Notch1 specific antibodies to detect the presence of Notch1. Preferably, the biological sample is preferably tissue sample or biopsies of human origin which can be conveniently assayed for the presence of a pathological hyperproliferative oncogenic disorder associated with expression of Notch1.

Once a determination is made of the amount of Notch1 present in the test sample, the results can be compared with those of control samples, which are obtained in a manner similar to the test samples but from individuals that do not have or present with a hyperproliferative oncogenic disorder associated with expression of Notch1, e.g., ovarian cancer. If the level of the Notch1 receptor polypeptide is significantly elevated in the test sample, it may be concluded that there is an increased likelihood of the subject from which it was derived has or will develop said disorder, e.g., T-ALL. The diagnostic uses of the antibodies according to the present invention embrace primary tumors and cancers, as well as metastases. Preferably, the antibody, or one of its functional fragments, can be present in the form of an immunoconjugate or of a labeled antibody so as to obtain a detectable and/or quantifiable signal.

An exemplary in vitro method of diagnosing pathological hyperproliferative oncogenic disorder comprises: (a) determining the presence or absence of Notch1 bearing cells in a sample; and (b) diagnosing a pathological condition or a susceptibility to a pathological condition based on the presence or absence of said Notch1 bearing cells. In the clinical diagnosis or monitoring of patients with an Notch1 mediated neoplastic disease, the detection of Notch1 expressing cells or an increase in the levels of Notch1, in comparison to the levels in a corresponding biological sample from a normal subject or non-cancerous tissue is generally indicative of a patient with or suspected of presenting with an Notch1 mediated disorder.

A representative in vitro method of diagnosing the presence of cancer in a patient or a susceptibility to a pathological condition associated therewith in a subject, proposes: (a) measuring the levels of Notch1 receptor protein in cells or tissues of the patient; and (b) comparing the measured levels of the antigen of (a) with levels of the antigen (Notch1 receptor protein) in cells or tissues from a normal human control, wherein an increase in the measured levels of the antigen in the patient versus the normal control is associated with the presence of the cancer. In certain embodiments, decreased Notch1 expression may be diagnostic of a pathological condition such as disorders of the skin. Alternatively, one may measure the level of the ICD as a measure of Notch receptor activation.

Alternatively, the above method may be practiced over several time points. A representative embodiment thus provides a method of diagnosing a pathological oncogenic disorder associated with aberrant expression of Notch 1 or increased Notch1 receptor activation (Notch cascade) comprising the steps of: a) detecting the presence and level of Notch1 in a biological sample obtained from the mammal at a plurality of time points, wherein Notch1 is detected by a method selected from the group consisting of immunoblotting, western blotting, immunoperoxidase staining, fluorescein labeling, diaminobenzadine and biotinylation; and b) correlating change in Notch1 expression with said diagnosis. It is understood that other conventional assays may be used instead of or in addition to those described herein.

A method of monitoring metastatic potential of an oncogenic disorder associated with Notch1 expression in a mammal is also encompassed. In accordance therewith, provided herein is a method for screening for metastatic potential of solid tumors comprising: a) obtaining a sample of tumor tissue from an individual in need of screening for metastatic potential of a solid tumor; b) reacting an antibody to Notch1 with tumor tissue from the patient; c) detecting the extent of binding of the Notch1 antibody to the tissue and d) correlating the extent of binding of the antibody with its metastatic potential. Preferably, the tumor is cancer arising from large bowel (colorectal cancer), prostate, breast or skin (melanoma or T-ALL). In certain embodiments, step c) may be performed over a plurality of time points. As well, in certain embodiments, Notch1 expression is detected by a method selected from the group consisting of immunohistochemical staining, immunoblotting, western blotting, immunoperoxidase staining, fluorescein labeling, diaminobenzadine and biotinylation.

The invention further provides for a method for predicting susceptibility to cancer comprising detecting the expression level of Notch1 in a tissue sample, its presence indicating susceptibility to cancer, wherein the degree of Notch1 expression correlates to the degree of susceptibility. In certain embodiments, the expression of Notch1 in, for example, breast tissue, prostate tissue, colon tissue, or any other tissue suspected of cells expressing Notch1 is examined, with the presence of Notch 1 in the sample providing an indication of cancer susceptibility or the emergence or existence of a tissue specific tumor.

Stage determination has potential prognostic value and provides criteria for designing optimal therapy. Simpson et al., J. Clin. Oncology 18:2059 (2000). Generally, pathological staging of breast cancer for example, is preferable to clinical staging because the former gives a more accurate prognosis. However, clinical staging would be preferred if it were as accurate as pathological staging because it does not depend on an invasive procedure to obtain tissue for pathological evaluation. Thus, methods for gauging tumor aggressiveness are also provided as are methods for observing the progression of a malignancy in an individual over time.

Accordingly, the invention provides an in vivo imaging reagent comprising an antibody according to the invention, or one of its functional fragments, preferably labeled, especially radiolabeled, and its use in medical imaging, in particular for the detection of Notch1 mediated disorders e.g., cancer characterized by over expressing Notch1 or other pathologies in which cells over express Notch1.

The imaging reagents, e.g., diagnostic reagents can be administered by intravenous injection into the body of the patient, or directly into a tissue suspected of harboring Notch1 bearing cells, e.g., colon or ovary or the pancreas. The dosage of reagent should be within the same ranges as for treatment methods. Typically, the reagent is labeled, although in some methods, the primary reagent with affinity for Notch1 is unlabelled and a secondary labeling agent is used to bind to the primary reagent. The choice of label depends on the means of detection. For example, a fluorescent label is suitable for optical detection. Use of paramagnetic labels is suitable for tomographic detection without surgical intervention. Radioactive labels can also be detected using PET or SPECT.

Diagnosis is performed by comparing the number, size, and/or intensity of labeled loci, to corresponding baseline values. The baseline values can, as an example, represent the mean levels in a population of undiseased individuals. Baseline values can also represent previous levels determined in the same patient. For example, baseline values can be determined in a patient before beginning treatment, and measured values thereafter compared with the baseline values. A decrease in values relative to baseline signals a positive response to treatment.

Thus, a general method embodied by the invention works by administering to a patient an imaging-effective amount of an imaging reagent such as the above described monoclonal antibodies or antigen-binding fragments which are labeled and a pharmaceutically effective carrier and then detecting the agent after it has bound to Notch1 present in the sample.

In certain embodiments, the method works by administering an imaging-effective amount of an imaging agent comprising a targeting moiety and an active moiety. The targeting moiety may be an antibody, Fab, Fab′2, a single chain antibody or other binding agent that interacts with an epitope present in Notch1. The active moiety may be a radioactive agent, such as radioactive technetium, radioactive indium, or radioactive iodine. The imaging agent is administered in an amount effective for diagnostic use in a mammal such as a human and the localization and accumulation of the imaging agent is then detected. The localization and accumulation of the imaging agent may be detected by radionuclide imaging, radioscintigraphy, nuclear magnetic resonance imaging, computed tomography, positron emission tomography, computerized axial tomography, X-ray or magnetic resonance imaging method, fluorescence detection, and chemiluminescent detection.

The in vivo imaging methods of the present invention are also useful for providing prognoses to cancer patients. For example, the presence of Notch1 indicative of an aggressive cancer likely to metastasize or likely to respond to a certain treatment can be detected. Thus, in one aspect, the invention provides a method for observing the progression of a malignancy in an individual over time comprising determining the level of Notch1 expressed by cells in a sample of the tumor, comparing the level so determined to the level of Notch1 expressed in an equivalent tissue sample taken from the same individual at a different time, wherein the degree of Notch1 expression in the tumor sample over time provides information on the progression of the cancer.

The in vivo imaging methods of the present invention can further be used to detect Notch1 mediated cancers e.g., one that has metastasized in other parts of the body.

A related embodiment relates to a pharmaceutical composition for in vivo imaging of an oncogenic disorder associated with expression of Notch1 comprising the invention antibodies or binding fragment thereof which is labeled and which binds Notch1 in vivo; and a pharmaceutically acceptable carrier.

The antibodies disclosed herein may also be used in methods of identifying human tumors that can escape anti-Notch1 treatment by observing or monitoring the growth of the tumor implanted into a rodent or rabbit after treatment with a conventional anti-Notch1 antibody.

The antibodies of the invention can also be used to study and evaluate combination therapies with anti-Notch1 antibodies of this invention and other therapeutic agents. The antibodies and polypeptides of this invention can be used to study the role of Notch1 in other diseases by administering the antibodies or polypeptides to an animal suffering from the disease of a similar disease and determining whether one or more symptoms of the disease are alleviated.

Those of skill in the art are very familiar with differentiating between significant expression of a target antigen, e.g., Notch1, which represents a positive identification, and low level or background expression of the antigen. Indeed, background expression levels are often used to form a “cut-off” above which increased staining will be scored as significant or positive. Significant expression may be represented by high levels of antigens in target cells or tissues or alternatively, by a high proportion of cells from within a tissue that each give a positive signal.

The above diagnostic approaches can be combined with any one of a wide variety of prognostic and diagnostic protocols known in the art. For example, another embodiment of the invention is directed to methods for observing a coincidence between the expression of Notch1 and a factor that is associated with malignancy, as a means for diagnosing and prognosticating the status of a tissue sample. A wide variety of factors associated with malignancy can be utilized, such as the expression of genes or gene products associated with malignancy (e.g. ICD, Hes1, Numb, Hes5 etc) as well as gross cytological observations. Methods for observing a coincidence between the expression of Notch1 and another factor that is associated with malignancy are useful, for example, because the presence of a set of specific factors that coincide with disease provides information crucial for diagnosing and prognosticating the status of a tissue sample. The methods proposed herein can be useful to diagnose or confirm diagnosis of an oncogenic disorder associated with expression of Notch1 or susceptibility thereof. For example, the methods can be used on a patient presenting with symptoms of an oncogenic disorder. If the patient has, for example increased expression levels of Notch1 or aberrant Notch receptor activation as evidenced by increased expression levels of any one or more downstream targets effected by or related to Notch receptor activation or increased expression levels of ICD, then the patient is likely suffering from a cancerous disorder. The methods can also be used in asymptomatic patients. Presence of higher than normal Notch1 may indicate for example susceptibility to future symptomatic disease. As well, the methods are useful for monitoring progression and/or response to treatment in patients who have been previously diagnosed with an Notch1 mediated cancer.

Generally speaking, malignancies are characterized by either increased Notch1 receptor expression, increased or aberrant Notch receptor activation or mutations resident in the Notch1 receptor protein. Malignancies characterized by aberrant or increased Notch receptor activation may be confirmed via determination of expression levels of ICD, whose expression level may be increased in the cytoplasm following activation of the Notch cascade. Thus, in those cases where a malignancy is characterized by increased Notch receptor activation, one is expected to find increased expression of ICD in the cytoplasm. This increase in ICD expression can be traced to the translocation of the ICD into the cytoplasm upon Notch receptor activation. A similar effect should be observed for downstream targets effected by aberrant Notch receptor activation—a decrease or increase relative to normal of a specific downstream Notch target occurring as a result of Notch receptor activation. Thus, measurement of Notch1 in biopsied tissue or other biological sample can be corroborated by determining expression of downstream target expression as a means of identifying high risk patients. In certain embodiments, single or multiple determinations of increased Notch1 expression and/or ICD expression over time may serve as a marker for illness indicative of intervening medical intervention. A positive test can therefore supplement the clinician's judgment.

Increased Notch1 levels also add prognostic accuracy to established severity of illness scores. Such clinical judgments will benefit by a method of scoring diseased cells. As discussed herein, measurement of Notch1 expression in a tissue sample can also be used as an indicator for additional monitoring or testing, or consideration for more aggressive treatment, especially when patients are found to have increased Notch1 expression levels or increased Notch receptor activation as reflected by increased cytoplasmic ICD levels or any other downstream target.

Thus, for example, one would expect that a patient at risk of developing a Notch mediated cancer or presenting with such a cancer would likely have increased Notch1 expression relative to a control sample. As such, in certain embodiments, for such patients, a semiquantitative estimation of Notch1 immunoreactivity can be performed. Towards this end, a score can be given to each slide, considering the intensity of the stain. The slides may be examined and scored independently by two investigators, and discordances may be reconciled by re-examination of the slide, and the scores then averaged. The intensity of immunostaining of individual cells may be scored on a scale of 0 (no staining) to 4 (strongest intensity) and the percentage of cells with staining at each intensity estimated. If there is no staining, a 0 score can be given. A +1 score indicates weak staining, while a +4 score indicating strong intensity of staining. As will be appreciated, any scoring scheme used to compare staining intensities may be used as long as it takes into account the relative intensity of cytoplasmic staining and allows differentiation among degrees of intensity of staining, thus providing a way to grade the malignancies. Because of the novel staining aspects of the present invention which results in highly differentiated staining, the scoring or grading can be done visually, thus allowing the technique of the present invention to be widely used clinically without sophisticated equipment. It will be understood that the staining results can be analyzed by appropriate sensitive optical equipment and analyzed by computer.

In furtherance of the above objective, the invention provides a method for diagnosing an oncogenic disorder associated with expression of Notch1 comprising: a) measuring by radioimmunoassay, competitive-binding assay, Western blot analysis, ELISA assay, or sandwich assay the amount of Notch1 protein in a biological sample, e.g., biopsied tissue obtained from a patient, using an antibody that specifically binds to Notch1; and b) comparing the amount of antibody bound to said Notch1 protein to a normal control tissue sample, wherein increased expression or over-expression of Notch1 in the sample obtained from the patient relative to the normal control tissue sample is diagnostic of an oncogenic disorder associated with expression of Notch1. Preferably, the Notch1-specific antibody comprises at least one antibody detailed herein.

In certain embodiment, the same scoring criteria e.g., score of 0 to 4 may be used to score cytoplasmic ICD staining as a means of corroborating the initial diagnosis. Consequently, cells may be stained with an antibody specific for ICD and the intensity level scored using the above criteria, where the intensity of immunostaining of individual cells may be scored on a scale of 0 (no staining) to 4 (strongest intensity) and the percentage of cells with staining at each intensity estimated. If there is no staining, a 0 score can be given. A +1 score indicates weak staining, while a +4 score indicating strong intensity of staining.

In additional embodiments, a prognostic index is produced by preparing a weighted scale of expression levels of the tumor markers, Notch1 and/or Notch1+Notch3 related to progression observed in a representative sample of a particular tumor type, wherein the different values in the weighted scale are related to increased invasiveness or metastatic spread in the representative sample.

The methods of the invention are also useful for identifying a human cancer patient at risk for additional neoplastic disease, for staging malignant disease in a human cancer patient and assessing the relative risk of metastatic disease versus the risk of toxicity (such as leukocytopenia, for example) from chemotherapeutic treatment.

The invention thus provides methods wherein the results of the determination of the level of cell surface Notch1 expression and the extent of cytoplasmic localization of ICD are used to prepare a prognostic or “risk” index for making a prognostic determination. In this aspect of the invention, a prognostic index is prepared using the above criteria, wherein a value of 0 signifies a control, a value of +1 indicates weak staining etc, wherein a prognosis of a likelihood of progressing to metastatic disease is made when the staining intensity is scoffed at +4.

An illustrative embodiment of the invention provides a diagnostic or monitoring method comprising: a) obtaining a sample of tissue from an individual in need of diagnosis or monitoring for cancer; b) detecting levels of Notch1 protein in said sample, c) scoring said sample for Notch1 protein levels; and d) comparing said scoring to that obtained from a control tissue sample to determine the prognosis associated with said cancer. Samples may be scored using a scale of 0 to 4, where 0 is negative (no detectable Notch1 expression or level comparable to a control level), and 4 is high intensity staining in the majority of cells and wherein a score of 1 to 4 indicates a poor prognosis while a score of 0 indicates a good prognosis.

A related aspect of the invention pertains to a method for screening for metastatic potential of a Notch1 mediated hyperproliferative disorder comprising: a) obtaining a sample of diseased or target tissue from an individual in need of screening for metastatic potential of a Notch1 mediated tumor, b) reacting an antibody to Notch1 with tumor tissue from the patient, c) detecting the extent of binding of the Notch1 antibody to said tissue and d) correlating the extent of binding of said antibody with its metastatic potential. In general, any of the methods of the invention involving analysis of the levels of Notch1 or ICD may be used in conjunction with additional cancer markers readily known to those of skill in the art.

Also provided is a method of detecting the presence and extent of cancer in a patient, comprising: determining the level of the antigen (Notch1) in a sample of cells or a tissue section from the patient and correlating the quantity of the antigen with the presence and extent of the cancer disease in the patient relative to a normal or control patient.

One of the major challenges facing the pharmaceutical industry in drug development is to show efficacy associated with a potential therapeutic candidate. This drawback applies equally to the numerous efforts underway in the pharmaceutical industry to generate anti-Notch1 inhibitory antibodies as anti-cancer therapeutics. One way to do this is to have a suitable marker that indicates when Notch1 activity is inhibited. Ideally, where a candidate Notch1 antagonist moiety is effective, one should observe a decrease in the expression levels of Notch1 following treatment with the Notch1 antagonist moiety. Alternatively, one might expect a decrease in the levels of ICD that is cleaved after Notch1 cascade is activated. It thus follows that favorable treatment with an Notch1 antagonistic moiety would predict a decrease in Notch1 expression levels on tumor cells or any other cells that express this cell surface receptor, while an unfavorable outcome would predict either no change in the expression levels or an increase in expression levels of Notch1. Thus, by measuring Notch1 protein expression on a tumor cell, for example, with a suitable marker, decreased expression levels may be detected as an indicator of suppressed Notch1 activity. The present invention exploits the ability of the Notch1 antibodies of the invention to bind Notch1 with high affinity to be utilized in a “biomarker strategy” for measuring Notch1 activity and/or expression or tumorigenic status by specifically measuring the expression levels of Notch1 on tumor/cancer cells. As a consequence, the present invention provides a rapid means, e.g., high affinity anti-Notch1 antibodies, for assessing the nature, severity and progression of a pathological hyperproliferative oncogenic disorder associated with expression of Notch1.

In furtherance of the “biomarker strategy” noted above, the invention provides a method for determining onset, progression, or regression, of neoplasias associated with expression of Notch1 in a subject, comprising: obtaining from a subject a first biological sample at a first time point, contacting the first sample with a effective amount of an antibody described herein under conditions allowing for binding of the antibody or a fragment thereof to Notch1 suspected to be contained in the sample and determining specific binding between the antibody in the first sample and Notch1 bearing cells to thereby obtain a first value, obtaining subsequently from the subject a second biological sample at a second time point, and contacting the second biological sample with the Notch1 antibody and determining specific binding between the antibody and Notch1 in said sample to obtain a second value, and comparing the determination of binding in the first sample to the determination of specific binding in the second sample as a determination of the onset, progression, or regression of the colon cancer, wherein an increase in expression level of Notch1 in said second or subsequent sample relative to the first sample indicative of the progression of said neoplasias, and wherein decrease in indicative of regression of neoplasias in said sample. The diagnosis or prognosis can be further corroborated, by correlating the expression levels of any one or more of various downstream targets effected by Notch receptor activation. In certain embodiments, the diagnosis or prognosis may be corroborated by measuring expression levels of ICD.

In one embodiment, Notch1 is detected by (1) adding an antibody of the invention to the sample or tissue section; (2) adding goat anti-mouse IgG antibody conjugated with peroxidase; (3) fixing with diaminobenzidene and peroxide, and (4) examining the sample or section, wherein reddish brown color indicates that the cells bear the antigen. According to the method, the effectiveness of a cancer treatment may be monitored by periodically measuring changes in the level of the antigen in a tissue sample taken from a patient undergoing the therapy, and correlating the change in level of the antigen with the effectiveness of the therapy, wherein a lower level of Notch1 expression determined at a later time point relative to the level of Notch1 determined at an earlier time point during the course of therapy indicates effectiveness of the therapy for the cancer disease. In an alternative embodiment, the levels of ICD in the cytoplasmic domain that is cleaved during the Notch cascade may be determined over time as an indication of the efficacy of the therapeutic protocol. Thus, a decrease in the level of the cytoplasmic domain ICD after treatment is indicative of a positive outlook and vice versa. In certain embodiments, levels of the ICD are measured over a plurality of time points.

An illustrative embodiment provides a method of diagnosis or prognosis comprising the steps of: examining the expression of ICD in a sample of diseased cells, e.g., breast tumor by immunohistochemistry, wherein a predominantly cytosolic staining indicates a lower likelihood of survival of the individual, wherein a lower cytosolic staining indicates a greater likelihood of survival.

In yet another embodiment, the application provides methods for determining the appropriate therapeutic protocol for a subject. Specifically, the antibodies of the invention will be very useful for monitoring the course of amelioration of malignancy in an individual, especially in those circumstances where the subject is being treated with a Notch1 antibody that does not compete with the antibodies of the invention for binding Notch1. Essentially, presence or absence or a change in the level of Notch1 expression may be indicative as to whether a subject is likely to have a relapse or a progressive, or a persistent neoplasias such as cancer associated with Notch1. Thus, by measuring an increase in the number of cells expressing Notch1 or changes in the concentration of Notch1 present in various tissues or cells, it is possible to determine whether a particular therapeutic regimen aimed at ameliorating a malignancy associated with Notch1 is effective. For those patients receiving conventional therapy, and whose Notch1 expression has not changed over time, for example, these may, instead be treated with the Notch1 antibodies of the invention and the change in Notch1 observed over time. If a change if readily evident over a period of time, then it may be possible to switch the patient from conventional therapy to therapy with one or more of the antibodies disclosed herein.

An antibody fragment of the invention is capable of specifically binding to a target molecule of interest. For example, in some embodiments, an antibody fragment specifically binds a tumor antigen. In some embodiments, the antibody fragment specifically binds a cell surface receptor that is activated upon receptor multimerization (e.g., dimerization). In some embodiments, binding of an antibody of the invention to a target molecule inhibits binding of another molecule (such as a ligand, where the target molecule is a receptor) to said target molecule.

Thus, in one example, an antibody fragment of the invention when bound to a target molecule inhibits binding of a cognate binding partner to the target molecule. A cognate binding partner can be a ligand, or a hetero or homodimerizing molecule. In one embodiment, an antibody fragment of the invention when bound to a target molecule inhibits target molecule receptor activation. For example, in some embodiments wherein an antibody or a fragment thereof is an antagonist, binding of the antibody fragment to a cell surface receptor may inhibit dimerization of the receptor with another unit of the receptor, whereby activation of the receptor is inhibited (due at least in part to a lack of receptor dimerization). In one embodiment, an antibody fragment of the invention is capable of competing with a native Notch receptor binding partner, e.g., delta or Serrate to the Notch1 receptor. In another embodiment, an antibody of the invention or a fragment thereof is capable of competing with an endogenous Notch receptor ligand for binding to a Notch1 receptor.

In certain embodiments, the herein described antibodies which antagonize, or inhibit, Notch function by either blocking or inhibiting Notch binding to its endogenous ligand or preventing or delaying Notch cascade activation (hereinafter “Antagonist Therapeutics Antibodies”) are administered for therapeutic effect. Disorders which can thus be treated can be identified by in vitro assays such as those described herein or known to one skilled in the art. Such Antagonist Antibodies include anti-Notch neutralizing antibodies, and competitive inhibitors of Notch protein-protein interactions as detailed infra. In furtherance of the above objective, an antibody of the invention is administered to treat a cancerous condition, or to prevent progression from a pre-neoplastic or non-malignant state into a neoplastic or a malignant state.

In one aspect, the invention provides an antibody or a fragment thereof that is an antagonist in single-armed form, but is an agonist or has agonist activity in a two-armed form (i.e., wherein the two arms have the same antigen binding capability).

Thus, in certain aspects, the invention provides a method for treating a disease comprising administering to a subject in need of such a treatment an effective amount for treating the disease of at least one antibody or antigenic or binding fragment thereof (“fragment”) disclosed herein that binds to native human Notch1 (hNotch1 or Notch1) and abolishes or attenuates the function of the native hNotch1 or hNotch receptor. A variety of diseases may be treated with the above described methods including cancer, in particular T-cell acute lymphoblastic leukemia/lymphoma, human breast cancer, human colorectal cancer, melanoma, human lung cancer, human head and neck cancers and human prostate cancer, an immune or inflammatory disorder like colitis or asthma, an infectious disease, an angiogenesis disorder, atherosclerosis, or a disorder of the kidney or any other disorder mediated by Notch signaling. Cardiovascular disease associated with Notch1 polymorphism as well as plasma disorders and neovascular disorders mediated by Notch1 receptor or aberrant receptor activation area also included.

Notch has also been implicated in regulating self-renewal of stem cells. Consequently, blocking notch signally by utilizing one or more of the invention antibodies will induce differentiation and sensitize Notch-bearing tumor cells to treatment with a Notch inhibitor. See, for example, WO 2008/108910, the entire content of which is incorporated by reference herein. See also “Notch signaling regulates stem cell numbers in vitro and in vivo” Nature 442, 823-826 (2006); Chica, S., Stem Cells 24:2437 (2006); Alexson, T. O. et al. (2006) Dev. Neurosci. 28:34 (2006); Shigeru, “Concise Review: Notch Signaling in Stem Cell Systems”. Stem Cells 24:2437-2447 (2006).

In yet another embodiment, there are provided Agonist Antibodies that promote Notch signaling cascade. A proposed use in accordance with this objective is using such agonist antibodies to treat skin cancer wherein the agonist antibodies described herein will decrease proliferation of skin cancer cells by activating the Notch receptor.

Yet another objective resides in the proposed use of constitutively active Notch receptor or an agonist thereof, for the purpose of developing a medicament that may find use in the treatment of a condition which is responsive to constitutively active Notch receptor or an agonist hereof.

In a further embodiment, the present invention provides a method of inhibiting or killing cancer cells, comprising: providing to a patient in need thereof the monoclonal antibody, or binding fragment thereof of the present invention, under conditions and in an amount sufficient for the binding to the cancer cells, thereby causing inhibition or killing of the cancer cells by the immune cells of the patient. Preferably, the method is for the treatment of T-cell acute lymphoblastic leukemia/lymphoma, human colon cancer, melanoma, human lung cancer and human prostate cancer. The monoclonal antibody is preferably conjugated with a cytotoxic moiety, such as a chemotherapeutic agent, a photoactivated toxin, a RNAi molecule or a radioactive agent. Preferably, the cytotoxic moiety may be a Ricin. An alternative method proposes treatment of a Notch1 mediated disorder comprising the steps as outlined above. Representative disorders include an immune or inflammatory disorder like colitis or asthma, an infectious disease, an angiogenesis disorder, atherosclerosis, or a disorder of the kidney or any other disorder mediated by Notch signaling.

In one embodiment, an oligonucleotide, such as an RNAi molecule inhibiting Notch1 expression may be conjugated to, or form the therapeutic agent portion of an immunoconjugate or antibody fusion protein of the present invention. Alternatively, the oligonucleotide may be administered concurrently or sequentially with a naked or conjugated anti-Notch1 antibody or antibody fragment of the present invention. In one embodiment, the oligonucleotides are an antisense oligonucleotide (RNAi) that preferably is directed against Notch1 expression.

An alternative embodiment provides a method of treating a Notch mediated disorder by administering a pharmaceutical composition comprising at least one Notch1 inhibitor wherein the inhibitor is an anti-Notch1 antibody conjugated to a Notch1 specific RNA inhibitor, and a pharmaceutically acceptable carrier. RNA inhibition (RNAi) is based on antisense modulation of Notch1 in cells and tissues comprising contacting the cells and tissues with at least one Notch1 antibody conjugated to a nucleic acid molecule that modulated transcription or translation of Notch1 receptor protein, including but not limited to double stranded RNA, (dsRNA), small interfering RNA (siRNA), ribozymes and locked nucleic acids (LNAs), and a pharmaceutically acceptable carrier.

The present invention further provides a method for localizing cancer cells in a patient, comprising: (a) administering to the patient a detectably-labeled monoclonal antibody of the invention, or binding fragment thereof; (b) allowing the detectably-labeled (e.g. radiolabeled; flurochrome labeled, or enzyme labeled, especially via ELISA) monoclonal antibody, or binding fragment thereof, to bind to the cancer cells within the patient; and (c) determining the location of the labeled monoclonal antibody or binding fragment thereof, within the patient.

Another embodiment of the invention relates to the use of invention antibodies, and VRs, FRs and CDRs thereof, in directed molecular evolution technologies such as phage display or bacterial or yeast cell surface display technologies in order to generate polypeptides with enhanced affinity, specificity, stability or other desired characteristics.

Another embodiment of the present invention is a cancer cell targeting diagnostic immunoconjugate comprising an antibody component that comprises an antibody or fragment thereof of any one of the antibodies or fragments thereof of the present invention, wherein the antibody, or fragment thereof is bound to at least one diagnostic/detection agent.

Preferably, the diagnostic/detection agent is selected from the group comprising a radionuclide, a contrast agent, and a photoactive diagnostic/detection agent. Still preferred, the diagnostic/detection agent is a radionuclide with an energy between 20 and 4,000 keV or is a radionuclide selected from the group consisting of ¹¹⁰In, ¹¹¹In, ¹⁷⁷Lu, ¹¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ⁹⁴mTc, ⁹⁹Tc, ⁹⁹mTc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ⁵²mMn, ⁵⁵Co, ⁷²As, ⁷⁵Br, ⁷⁶Br, ⁸²mRb, ⁸³Sr, or other gamma-, beta-, or positron-emitters. Also preferred, the diagnostic/detection agent is a paramagnetic ion, such as the a metal comprising chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), or a radioopaque material, such as barium, diatrizoate, ethiodized oil, gallium citrate, iocarmic acid, iocetamic acid, iodamide, iodipamide, iodoxamic acid, iogulamide, iohexyl, iopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, and thallous chloride.

Also preferred, the diagnostic/detection agent is a fluorescent labeling compound selected from the group comprising fluorescein isothiocyanate, rhodamine, phycoerytherin, phycocyanin, allophycocyanin, o-phthaldehyde and fluorescamine, a chemiluminescent labeling compound selected from the group comprising luminol, isoluminol, an aromatic acridinium ester, an imidazole, an acridinium salt and an oxalate ester, or a bioluminescent compound selected from the group comprising luciferin, luciferase and aequorin. In another embodiment, the diagnostic immunoconjugates of the present invention are used in intraoperative, endoscopic, or intravascular tumor diagnosis.

Another embodiment of the present invention is a cancer cell targeting therapeutic immunoconjugate comprising an antibody component that comprises an antibody or fragment thereof of any one of the antibodies, fusion proteins, or fragments thereof of the present invention, wherein the antibody, fusion protein, or fragment thereof is bound to at least one therapeutic agent.

Preferably, the therapeutic agent is selected from the group consisting of a radionuclide, an immunomodulator, a hormone, a hormone antagonist, an enzyme, oligonucleotide, an enzyme inhibitor, a photoactive therapeutic agent, a cytotoxic agent, an angiogenesis inhibitor, and a combination thereof.

In one embodiment, the therapeutic agent is a cytotoxic agent, such as a drug or a toxin. Also preferred, the drug is selected from the group consisting of nitrogen mustards, ethylenimine derivatives, alkyl sulfonates, nitrosoureas, gemcitabine, triazenes, folic acid analogs, anthracyclines, taxanes, COX-2 inhibitors, pyrimidine analogs, purine analogs, antibiotics, enzymes, enzyme inhibitors, epipodophyllotoxins, platinum coordination complexes, vinca alkaloids, substituted ureas, methyl hydrazine derivatives, adrenocortical suppressants, hormone antagonists, endostatin, taxols, camptothecins, SN-38, doxorubicins and their analogs, antimetabolites, alkylating agents, antimitotics, antiangiogenic, apoptotoic agents, methotrexate, CPT-11, and a combination thereof.

In another embodiment, the therapeutic agent is an oligonucleotide. For example, the oligonucleotide may be an antisense oligonucleotide such as an antisense oligonucleotide against Notch 1 or an RNAi molecule against Notch1 receptor expression.

In another embodiment, the therapeutic agent is a toxin selected from the group consisting of ricin, abrin, alpha toxin, saporin, ribonuclease (RNase), DNase I, Staphylococcal enterotoxin-A, pokeweed antiviral protein, gelonin, diphtherin toxin, Pseudomonas exotoxin, and Pseudomonas endotoxin and combinations thereof, an immunomodulator is selected from the group consisting of a cytokine, a stem cell growth factor, a lymphotoxin, a hematopoietic factor, a colony stimulating factor (CSF), an interferon (IFN), a stem cell growth factor, erythropoietin, thrombopoietin and a combinations thereof, a radionuclide selected from the group consisting of ³²P, ³³P, ⁴⁷Sc, ⁶⁴Cu, ⁶⁷Cu, ⁶⁷Ga, ⁸⁶Y, ⁹⁰Y, ¹¹¹Ag, ¹¹¹In, ¹²⁵U, ¹³¹I, ¹⁴²Pr, ¹⁵³Sm, ¹⁶¹Tb, ¹⁶⁶Dy, ¹⁶⁶Ho, ¹⁷⁷Lu, ¹⁸⁶Re, ¹⁸⁸Re, ¹⁸⁹Re, ²¹²Pb, ²¹²Bi, ²¹³Bi, ²¹¹At, ²²³Ra and ²²⁵Ac, and combinations thereof, or a photoactive therapeutic agent selected from the group comprising chromogens and dyes.

Still preferred, the therapeutic agent is an enzyme selected from the group comprising malate dehydrogenase, staphylococcal nuclease, delta-V-steroid isomerase, yeast alcohol dehydrogenase, α-glycerophosphate dehydrogenase, triose phosphate isomerase, horseradish peroxidase, alkaline phosphatase, asparaginase, glucose oxidase, β-galactosidase, ribonuclease, urease, catalase, glucose-6-phosphate dehydrogenase, glucoamylase and acetylcholinesterase.

A further aspect of the invention pertains to kits. The invention is a kit comprising a container housing Notch1 antibody or fragment thereof or antibody containing composition and instructions for administering the components in the kit to a subject at risk of, or in need of, treatment of a disease. The kit may further comprise a container housing a pharmaceutical preparation diluent.

The kit may also be used for determining whether an embedded biological sample contains human Notch1 protein comprising: (a) an Notch1-binding agent that specifically binds with an embedded human Notch1 protein to form a binding complex; and (b) an indicator capable of signaling the formation of said binding complex, wherein the Notch1 binding agent is a monoclonal antibody or a binding fragment thereof as set forth in the application. Diagnostic procedures using anti-Notch1 antibody of the invention can be performed by diagnostic laboratories, experimental laboratories, practitioners, or private individuals. The clinical sample is optionally pre-treated for enrichment of the target being tested for. The user then applies a reagent contained in the kit in order to detect the changed level or alteration in the diagnostic component

In a further embodiment, the invention concerns an article of manufacture, comprising: a container; a label on the container; and composition comprising an active agent contained within the container; wherein the composition is effective for the detection, diagnosis or prognosis of neoplasia associated with expression of Notch1 and the label on the container indicates that the composition can be used for the diagnosis or the prognosis of conditions characterized by overexpression of the Notch1 protein receptor.

The invention further pertains to an article of manufacture comprising a container and a composition contained within said container, wherein the composition includes an antibody as described herein.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

Other characteristics and advantages of the invention appear in the continuation of the description with the examples and the Figures whose legends are represented below.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

FIG. 1 shows a Western blot example of Notch1 expressing stable cell lines. Kinetic expression of stably expressed human full-length Notch1 as functions of time and tetracycline concentration, respectively, was conducted where optimal dose/time effect for the Notch lines was identified.

FIG. 2 shows an IHC staining example of a Notch1 expressing stable cell line. The human full-length Notch1 expressing stable cell lines were analyzed by IHC.

FIG. 3 shows a diagram of panning procedures used to identify novel Notch-specific antibodies.

FIG. 4 Phage displayed ScFv (N153-147) binding on Notch 1 on cells detected in FACS. Dotted line shows binding on 293 TRex control cells and solid trace line shows binding to Notch 1 expressing 293 TRex cells.

FIG. 5 shows purified Notch IgG by SDS-PAGE analysis following expression and purification in mammalian cells.

FIG. 6 displays binding affinity determination (EC50) in ELISA using different concentrations of purified IgG protein. (A) Three mAbs binding to Notch 1 ligand binding domain antigen (Ala19-Gln526); (B) Two mAbs binding to Notch 1 LNR-HD domain protein

FIG. 7 shows Notch 1 mAb blocking Notch1/Fc and Notch1 ligand interaction. IgG is the isotype non-blocker control and four Notch 1 ligand blocking Mabs are shown as indicated.

FIG. 8 shows the FACS analysis of Notch antibodies binding to (A) 293 FlpIn (TREx) cells stably expressing the different Notch paralogs (i.e., Notch1, Notch-2, Notch-3, and Notch-4) and (B) LS-1034 and BxPC3 cancer cell lines.

FIG. 9 shows the effect of Notch mAbs on the Jag-2-induced luciferase reporter gene activity in U2O5 FlpIn TREx cells stably expressing (A) hNotch1, (B) hNotch-2, (C) hNotch-3 chimera receptors, and (D) mNotch1 receptor. (E) Potency (IC₅₀) determined from the sigmoid curves generated by serial dilution of the Mabs tested in the Notch1-Gal4 reporter assay. IC₅₀ values were calculated using a 3-parameter logistic curve fit with the top fixed at 100. (F) Summary of the monoclonal antibody activity (IC50s<150 nM) as determined by the Notch1, −2 and -3 Gal4 reporter signaling assays and FACS analysis. Antibodies according to the present invention were able to inhibit Notch1 signaling as indicated by a decrease of luciferase activity in the reporter assay. Data are represented as luciferase fold stimulation in ligand-activated Notch cells relative to Notch cells co-cultivated with parental 3T3 cells (no ligand).

FIG. 10 displays a strategy adopted to generate the 10 k Membranome phage-Ab collection.

FIG. 11 shows a scatter plot of hybridization data obtained from incubating the Cy-5 and Cy-3 labelled tag probes on the micro-Array_{—}44 k chip where complementary tag sequences were immobilized. Hybridization was quantified by a fluorescence scanner. Results reported were obtained by combining data from two independent fluor reversal experiments. Orange dots indicate signals with log₂ ratio>2.7 and log₂ (average intensity>10.5. Red big dots indicate the 8 phage-Ab selected dots.

FIG. 12 shows the effect of Notch mAbs on ligand-independent spontaneous activation of Notch1 receptors bearing Class I (i.e., promote heterodimer dissociation) or Class II (i.e., tandem insertions that duplicate the S2 cleavage) mutations in the negative regulatory region (NRR) of the receptor. Antibodies according to the present invention were able to inhibit Notch1 signaling to the Class I (A), but not to the Class II (B) mutations as indicated by a decrease of luciferase activity in the reporter assay. Data are represented as luciferase fold stimulation observed in cells bearing Notch mutations relative to cells bearing the wild-type form of the receptor.

### DETAILED DESCRIPTION OF THE INVENTION

Provided herein are various human Notch1 and Notch1/Notch3 specific antibodies, preferably monoclonal antibodies. Included are antagonist, inhibitory and neutralizing anti-Notch1 antibodies that inhibit or decrease cancer cell growth or proliferation. Compositions comprising one or more of the herein described antibodies effective for use in the treating Notch1 mediated hyper-proliferative disorders are also included. A Notch receptor antagonist includes antigen-binding fragments thereof that bind the Notch receptor extracellularly and is effective in blocking cleavage of the receptor or activating the Notch receptor mediated signaling cascade. The compositions can be provided in an article of manufacture or a kit.

Also provided are agonist anti-notch1 antibodies. A Notch receptor agonist includes any molecule that can produce an activated signaling form of the receptor, including soluble ligands of the receptor, antibodies and fragments thereof that bind the Notch receptor extracellularly and have enzymatic activity in cleaving the receptor to release the activated form.

Another aspect of the invention is an isolated nucleic acid encoding any one or more of the anti-Notch1 antibodies of the invention, as well as a vector comprising the nucleic acid. The human Notch1 DNA sequence can be found using GenBank Accession Number (NP-060087). Methods of recombinant production of the invention antibodies are also within the scope o the invention. Another aspect of the invention is a method of inhibiting or decreasing the proliferation of cancer cells by administering a Notch1 antibody which results in blocking of the endogenous ligand to the Notch1 receptor or inactivation/deactivation of Notch signaling. Another aspect of the invention is a method of destroying cancer and tumor cells which express a Notch receptor by administering to a patient in need thereof, a therapeutically effective amount of a composition comprising a Notch receptor binding partner, e.g., any one or more of the Notch specific antibodies disclosed herein effective for that purpose. A further aspect of the invention is a method of alleviating cancer by administering an agonist or antagonist of Notch receptor. For therapeutic applications, the modulators of Notch signaling can be used alone, or in combination therapy with, e.g., hormones, antiantiogens, or radiolabeled compounds, or with surgery, cryotherapy, and/or radiotherapy.

The Notch receptor binding partner useful in destroying cancer cells includes soluble ligands of the receptor, antibodies and fragments thereof that bind the Notch receptor. The binding partners can be conjugated to a cytotoxic agent. The antibodies are preferably growth inhibitory antibodies. The cytotoxic agent can be a toxin, antibiotic, radioactive isotope or nucleolytic enzyme. A preferred cytotoxic agent is a toxin, preferably a small molecule toxin such as calicheamicin or a maytansinoid.

The agonists/antagonists and binding partners of Notch receptor can be synthetically or recombinantly produced or otherwise isolated.

The mention of particular references, patent application and patents throughout this application should be read as being incorporated by reference into the text of the specification.

### Definitions

Before the present proteins, nucleotide sequences, and methods are described, it is to be understood that the present invention is not limited to the particular methodologies, protocols, cell lines, vectors, and reagents described, as these may vary. It is also understood that the terminology used herein is for the purpose of describing particular embodiments only, and is not to limit the scope of the present invention.

The singular forms “a,” “an,” and “the” include plural reference unless the context clearly dictates otherwise.

All technical and scientific terms used herein have the same meanings as commonly understood by one of ordinary skill in the art to which this invention pertains. The practice of the present invention will employ, unless otherwise indicated, conventional techniques of protein chemistry and biochemistry, molecular biology, microbiology and recombinant DNA technology, which are within the skill of the art. Such techniques are explained fully in the literature.

Although any machines, materials, and methods similar or equivalent to those described herein can be used to practice or test the present invention, the preferred machines, materials, and methods are now described. All patents, patent applications, and publications mentioned herein, whether supra or infra, are each incorporated by reference in its entirety.

Terms used throughout this application are to be construed with ordinary and typical meaning to those of ordinary skill in the art. However, Applicants desire that the following terms be given the particular definition as defined below:

Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

“Nucleic acid” or a “nucleic acid molecule” as used herein refers to any DNA or RNA molecule, either single- or double-stranded and, if single-stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. In some embodiments of the invention, nucleic acids are “isolated.” This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

Similarly, “amino acid sequence” as used herein refers to an oligopeptide, peptide, polypeptide, or protein sequence and fragments or portions thereof, of a naturally occurring or synthetic molecule.

The terms “isolated”, “purified”, or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein or nucleic acid that is the predominant species present in a preparation is substantially purified. In particular, an isolated nucleic acid is separated from some open reading frames that naturally flank the gene and encode proteins other than protein encoded by the gene. The term “purified” in some embodiments denotes that a nucleic acid or protein gives rise to essentially one band in an electrophoretic gel. Preferably, it means that the nucleic acid or protein is at least 85% pure, more preferably at least 95% pure, and most preferably at least 99% pure. “Purify” or “purification” in other embodiments means removing at least one contaminant from the composition to be purified. In this sense, purification does not require that the purified compound be homogenous, e.g., 100% pure

Nucleic acid is “operably linked” when it is placed into a functional relationship with another nucleic acid sequence. This can be a gene and a regulatory sequence(s) which are connected in such a way as to permit gene expression when the appropriate molecules (e.g., transcriptional activator proteins) are bound to the regulatory sequences(s). For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous Linking is accomplished by ligation at convenient restriction sites. If such sites do not exist; the synthetic oligonucleolide adaptors or linkers are used in accordance with conventional practice.

The term “cell”, “cell line” and “cell culture” are used interchangeably, and all such designations include progeny. It is also understood that all progeny may not be precisely identical in DNA content, due to deliberate or inadvertent mutations. Mutant progeny that have the same function or biological property, as screened for in the originally transformed cell, are included.

The “host cells” used in the present invention generally are prokaryotic or eukaryotic hosts. Examples of suitable host cells are described in Section B. Vectors, Host Cells and Recombinant Methods: (vii) Selection and transformation of host cells.

“Transformation” means introducing DNA into an organism so that the DNA is replicable, either as an extrachronosomal element or by chromosomal integration.

“Transfection” refers to the taking up of an expression vector by a host cell whether or not any coding sequences are in fact expressed.

The terms “transfected host cell” and “transformed” refer to the introduction of DNA into a cell. The cell is termed “host cell” and it may be either prokaryotic or eukaryotic. Typical prokaryotic host cells include various strains of E. coli. Typical eukaryotic host cells are mammalian, such as Chinese hamster ovary or cells of human origin. The introduced DNA sequence may be from the same species as the host cell or a different species from the host cell, or it may be a hybrid DNA sequence, containing some foreign and some homologous DNA.

The terms “replicable expression vector” and “expression vector” refer to a piece of DNA, usually double-stranded, which may have inserted into it a piece of foreign DNA. Foreign DNA is defined as heterologous DNA, which is DNA not naturally found in the host cell. The vector is used to transport the foreign or heterologous DNA into a suitable host cell. Once in the host cell, the vector can replicate independently of the host chromosomal DNA and several copies of the vector and its inserted (foreign) DNA may be generated.

The term “vector” means a DNA construct containing a DNA sequence which is operably linked to a suitable control sequence capable of effecting the expression of the DNA in a suitable host. Such control sequences include a promoter to effect transcription, an optional operator sequence to control such transcription, a sequence encoding suitable mRNA ribosome binding sites, and sequences which control the termination of transcription and translation. The vector may be a plasmid, a phage particle, or simply a potential genomic insert. Once transformed into a suitable host, the vector may replicate and function independently of the host genome, or may in some instances, integrate into the genome itself. In the present specification, “plasmid” and “vector” are sometimes used interchangeably, as the plasmid is the most commonly used form of vector at present. However, the invention is intended to include such other form of vector which serve equivalent function as and which are, or become, known in the art. Typical expression vectors for mammalian cell culture expression, for example, are based on pRK5 (EP 307,247), pSV16B (WO 91/08291), and pVL1392 (Pharmingen).

The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

The terms “protein” or “polypeptide” are intended to be used interchangeably. They refer to a chain of two (2) or more amino acids which are linked together with peptide or amide bonds, regardless of post-translational modification (eg., glycosylation or phosphorylation). Antibodies are specifically intended to be within the scope of this definition. The polypeptides of this invention may comprise more than one subunit, where each subunit is encoded by a separate DNA sequence.

Amino acids may be referred to herein either by their commonly known three letter symbols or by the one-letter symbols recommended by the IUPAC-IUB Biochemical Nomenclature Commission. Nucleotides, likewise, may be referred to by their commonly accepted single-letter codes.

The phrase “substantially identical” with respect to an antibody polypeptide sequence shall be construed as an antibody exhibiting at least 70%, preferably 80%, more preferably 90% and most preferably 95% sequence identity to the reference polypeptide sequence. The term with respect to a nucleic acid sequence shall be construed as a sequence of nucleotides exhibiting at least about 85%, preferably 90%, more preferably 95% and most preferably 97% sequence identity to the reference nucleic acid sequence. For polypeptides, the length of the comparison sequences will generally be at least 25 amino acids. For nucleic acids, the length will generally be at least 75 nucleotides. Methods and computer programs for the alignment are well known in the art. Sequence identity may be measured using sequence analysis software (e.g, Sequence Analysis Software Package, Genetics Computer Group, University of Wisconsin Biotechnology Center, 1710 University Ave., Madison, Wis. 53705). This software matches similar sequences by assigning degrees of homology to various substitutions, deletions, and other modifications.

The term “identity” or “homology” shall be construed to mean the percentage of amino acid residues in the candidate sequence that are identical with the residue of a corresponding sequence to which it is compared, after aligning the sequences and introducing gaps, if necessary to achieve the maximum percent identity for the entire sequence, and not considering any conservative substitutions as part of the sequence identity. A molecule is “substantially similar” to another molecule if both molecules have substantially similar structures or biological activity. Thus, provided that two molecules possess a similar activity, they are considered variants as that term is used herein even if the structure of one of the molecules is not found in the other, or if the sequence of amino acid residues is not identical. Neither N- or C-terminal extensions nor insertions shall be construed as reducing identity or homology

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site located at www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions, as well as naturally occurring, e.g., polymorphic or allelic variants, and man-made variants. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of one of the number of contiguous positions selected from the group consisting typically of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Preferred examples of algorithms that are suitable for determining percent sequence identity and sequence similarity include the BLAST and BLAST 2.0 algorithms. BLAST and BLAST 2.0 are used, with the parameters described herein, to determine percent sequence identity for the nucleic acids and proteins of the invention.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, e.g., where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequences.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG, and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. Typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The term “amino acid sequence variant” refers to a polypeptide that has amino acid sequences that differ to some extent from a native sequence polypeptide.

“Substitutional” “amino acid variant” refers to molecules with some differences in their amino acid sequences as compared to a native amino acid sequence. The substitutions may be single, where only one amino acid in the molecule as been substituted, or they may be multiple, where two or more amino acids have been substituted in the same molecule. Ordinarily, amino acid sequence variants of Notch receptor will possess at least about 70% homology with the native sequence Notch receptor, preferably, at least about 80%, more preferably at least about 85%, even more preferably at least about 90% homology, and most preferably at least 95%. The amino acid sequence variants can possess substitutions, deletions, and/or insertions at certain positions within the amino acid sequence of the native amino acid sequence. “Insertional” variants are those with one or more amino acids inserted immediately adjacent to an amino acid at a particular position in a native sequence. Immediately adjacent to an amino acid means connected to either the α-carboxyl or α-amino functional group of the amino acid. “Deletional” variants are those with one or more amino acids in the native amino acid sequence removed. Ordinarily, deletional variants will have one or two amino acids deleted in a particular region of the molecule.

The term “antibody” (Ab) as used herein includes monoclonal antibodies, polyclonal antibodies, multispecific antibodies (e.g. bispecific antibodies), and antibody fragments, so long as they exhibit the desired biological activity. The term “immunoglobulin” (Ig) is used interchangeably with “antibody” herein. An “isolated antibody” is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In certain embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The term “immunoglobulin” or “antibody” (used interchangeably herein) is used in the broadest sense and is meant to encompass an immunoglobulin molecule obtained by in vitro or in vivo generation of an immunogenic response. A broad scope refers to an antigen-binding protein having a basic four-polypeptide chain structure consisting of two heavy and two light chains, said chains being stabilized, for example, by interchain disulfide bonds, which has the ability to specifically bind antigen. Both heavy and light chains are folded into domains. The term “domain” refers to a globular region of a heavy or light chain polypeptide comprising peptide loops (e.g., comprising 3 to 4 peptide loops) stabilized, for example, by β-pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. “Constant” domains on the light chain are referred to interchangeably as “light chain constant regions”, “light chain constant domains”, “CL” regions or “CL” domains) “Constant” domains on the heavy chain are referred to interchangeably as “heavy chain constant regions”, “heavy chain constant domains”, “CH” regions or “CH” domains) “Variable” domains on the light chain are referred to interchangeably as “light chain variable regions”, “light chain variable domains”, “VL” regions or “VL” domains). “Variable” domains on the heavy chain are referred to interchangeably as “heavy chain variable regions”, “heavy chain variable domains”, “VH” regions or “VH” domains). Particular amino acid residues are believed to form an interface between the light and heavy chain variable domains (Clothia et al., J. Mol Biol. 186, 651-66, 1985); Novotny and Haber, Proc. Natl. Acad Sci. USA 82, 4592-4596 (1985). The term includes polyclonal, monoclonal, single chain and multivalent antibodies. Fragments e.g., Fab, Fab′, F(ab)2, Fv etc. are also included. Representative members include for example, single anti-Notch1 monoclonal antibodies (including agonist, antagonist, and neutralizing antibodies), anti-Notch1 antibody compositions with polyepitopic specificity (e.g. bispecific antibodies so long as they exhibit the desired biological activity), polyclonal antibodies, single chain anti-Notch1 antibodies, and fragments of anti-Notch1 antibodies as long as they exhibit the desired biological or immunological activity. The antibodies may be genetically engineered antibodies and/or produced by recombinant DNA techniques. Fully human antibodies can also be produced by phage display, gene and chromosome transfection methods, as well as by other means. The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated α, δ, ε, γ, and μ, respectively. The γ and α classes are further divided into subclasses on the basis of relatively minor differences in C_{H} sequence and function, e.g., humans express the following subclasses: IgG1, IgG2, IgG3, IgG4, IgA1, and IgA2.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, Nature 256, 495 (1975), or may be made by recombinant methods, e.g., as described in U.S. Pat. No. 4,816,567. The monoclonal antibodies for use with the present invention may also be isolated from phage antibody libraries using the techniques described in Clackson et al. Nature 352: 624-628 (1991), as well as in Marks et al., J. Mol. Biol 222: 581-597 (1991).

An “intact” antibody is one which comprises an antigen-binding site as well as a C_{L} and at least heavy chain constant domains, C_{H}1, C_{H}2 and C_{H}3. The constant domains may be native sequence constant domains (e.g. human native sequence constant domains) or amino acid sequence variant thereof. Preferably, the intact antibody has one or more effector functions.

The term “region” refers to a part or portion of an antibody chain and includes constant or variable domains as defined herein, as well as more discrete parts or portions of said domains. For example, light chain variable domains or regions include “complementarity determining regions” or “CDRs” interspersed among “framework regions” or “FRs”, as defined herein.

The term “antigen” as used herein, means a molecule which is reactive with a specific antibody.

“Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance.

Antibodies of “IgG class” refers to antibodies of IgG1, IgG2, IgG3, and IgG4. The numbering of the amino acid residues in the heavy and light chains is that of the EU index (Kabat, et al., “Sequences of Proteins of Immunological Interest”, 5^{th} ed., National Institutes of Health, Bethesda, Md. (1991); the EU numbering scheme is used herein).

An “immunogenic response” or “antigenic response” is one that results in the production of antibodies directed to a compound after the appropriate cells have been contacted therewith. The compound that is used to elicit an immunogenic response is referred to as an immunogen or antigen. The antibodies produced in the immunogenic response specifically bind the immunogen used to elicit the response.

The term “antibody mutant” refers to an amino acid sequence variant of an antibody wherein one or more of the amino acid residues have been modified. Such mutant necessarily have less than 100% sequence identity or similarity with the amino acid sequence having at least 75% amino acid sequence identity or similarity with the amino acid sequence of either the heavy or light chain variable domain of the antibody, more preferably at least 80%, more preferably at least 85%, more preferably at least 90%, and most preferably at least 95%. Since the method of the invention applies equally to both polypeptides, antibodies and fragments thereof, these terms are sometimes employed interchangeably.

Alternatively or additionally, the word “mutant”, as used herein, is interchangeable with “mutationally-altered” and “glycosylation site altered”. The terms refer to an antibody that comprises at least one immunoglobulin variable region containing at least one mutation that modifies a V region glycosylation site. A mutant immunoglobulin refers to an immunoglobulin (e.g., F(ab′)₂, Fv, Fab, bifunctional antibodies, antibodies, etc.) comprising at least one immunoglobulin variable region containing at least one mutation that modifies a V region glycosylation site. A mutant immunoglobulin chain has at least one mutation that modifies a V region glycosylation site, typically in the V region framework. Thus, the pattern (i.e., frequency and or location(s) of occurrence) of V region glycosylation sites is altered in a mutant immunoglobulin.

The term “variable” in the context of variable domain of antibodies, refers to the fact that certain portions of the variable domains differ extensively in sequence among antibodies and are used in the binding and specificity of each particular antibody for its particular antigen. The V domain mediates antigen binding and define specificity of a particular antibody for its particular antigen. However, the variability is not evenly distributed across the 110-amino acid span of the variable domains. It is concentrated in three segments called complementarity determining regions (CDRS) also known as hypervariable regions both in the light chain and the heavy chain variable domains. There are at least two techniques for determining CDRs: (1) an approach based on cross-species sequence variability (i.e., Kabat et al., Sequences of Proteins of Immunological Interest (National Institute of Health, Bethesda, Md. 1987); and (2) an approach based on crystallographic studies of antigen-antibody complexes (Chothia, C. el at. (1989), Nature 342: 877). The more highly conserved portions of variable domains are called the framework (FR) of 15-30 amino acids separated by shorter “hypervariable regions” (9-12 amino acids long). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies. See Kabat et al., Sequences of Proteins of Immunological Interest, 5th Ed. Public Health Service, National Institutes of Health, Bethesda, Md. (1991). The constant domains are not involved directly in binding an antibody to an antigen, but exhibit various effector function, such as participation of the antibody in antibody-dependent cellular toxicity.

The term “hypervariable region” when used herein refers to the amino acid residues of an antibody which are responsible for antigen-binding. The hypervariable region generally comprises amino acid residues from a “complementarity determining region” or “CDR” (e.g. around about residues 24-34 (Li), 50-56 (L2) and 89-97 (L3) in the V_{L}, and around about 1-35 (H1), 50-65 (H2) and 95-102 (H3) in the V_{H}; Kabate et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the V_{L}, and 26-32 (H1), 53-55 (H2) and 96-101 (H3) in the V_{H}; Chothia and Lesk J. Mol. Biol. 196:901-917 (1987)).

Immunoglobulins or antibodies can exist in monomeric or polymeric form. The term “antigen-binding fragment” refers to a polypeptide fragment of an immunoglobulin or antibody binds antigen or competes with intact antibody (i.e., with the intact antibody from which they were derived) for antigen binding (i.e., specific binding). The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof. Preferably, the fragment exhibits qualitative biological activity in common with a full-length antibody. For example, a functional fragment or analog of an anti-IgE antibody is one which can bind to an IgE immunoglobulin in such a manner so as to prevent or substantially reduce the ability of such molecule from having the ability to bind to the high affinity receptor, FcεRI. Antibody fragments can be prepared by in vitro manipulation of antibodies (e.g., by limited proteolysis of an antibody), or via recombinant DNA technology (e.g., the preparation of single-chain antibodies from phage display libraries). Examples of antibody fragments include Fab, Fab′, F(ab′)2, and Fv fragments; diabodies; linear antibodies (U.S. Pat. No. 5,641,870, Example 2; Zapata et al., Protein Eng. 8(10): 1057-1062 (1995)); single-chain antibody molecules; and multispecific antibodies formed from antibody fragments.

Binding fragments are produced by recombinant DNA techniques, or by enzymatic or chemical cleavage of intact immunoglobulins. Binding fragments include Fab, Fab′, F(ab′)2, Fabc, Fv, single chains, and single-chain antibodies. Other than “bispecific” or “bifunctional” immunoglobulins or antibodies, an immunoglobulin or antibody is understood to have each of its binding sites identical.

“A functional variant” of the antibody molecule according to the invention is an antibody molecule which possesses a biological activity (either functional or structural) that is substantially similar to the antibody molecule according to the invention, i.e. a substantially similar substrate specificity or cleavage of the substrate. The term “functional variant” also includes “a fragment”, “an allelic variant”, “variant based on the degenerative nucleic acid code” or “chemical derivatives”. Such a “functional variant” e.g. may carry one or several point mutations, one or several nucleic acid exchanges, deletions or insertions or one or several amino acid exchanges, deletions or insertions. Said functional variant is still retaining its biological activity such as antibody binding activity, at least in part or even going along with an improvement said biological activity.

An “allelic variant” is a variant due to the allelic variation, e.g. differences in the two alleles in humans. Said variant is still retaining its biological activity such as antibody target binding activity, at least in part or even going along with an improvement said biological activity.

A “variant based on the degenerative of the genetic code” is a variant due to the fact that a certain amino acid may be encoded by several different nucleotide triplets. Said variant is still retaining its biological activity such as antibody binding activity, at least in part or even going along with an improvement said biological activity.

A “fusion molecule” may be the antibody molecule according to the invention fused to e.g. a reporter such as a radiolabel, a chemical molecule such as a toxin or a fluorescent label or any other molecule known in the art.

As used herein, a “chemical derivative” according to the invention is an antibody molecule according to the invention chemically modified or containing additional chemical moieties not normally being part of the molecule. Such moieties may improve the molecule's activity such as target destruction (e.g. killing of tumor cells) or may improve its solubility, absorption, biological half life, etc.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site. Pepsin treatment of an antibody yields a single large F(ab′)2 fragment which roughly corresponds to two disulfide linked Fab fragments having divalent antigen-binding activity and is still capable of cross-linking antigen. Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, which region is also the part recognized by Fc receptors (FcR) found on certain types of cells. By “Fc” or “Fc region” as used herein is meant the polypeptides comprising the last two constant region immunoglobulin domains of IgA, IgD, and IgG, and the last three constant region immunoglobulin domains of IgE and IgM, and part of the flexible hinge N-terminal to these domains. Although the boundaries of the Fc region may vary, the human IgG heavy chain Fc region is usually defined to comprise residues C226 or P230 to its carboxyl-terminus, wherein the numbering is according to the EU numbering scheme. Fc may refer to this region in isolation, or this region in the context of a full length antibody or antibody fragment. Ergo, by “outside the Fc region” as used herein is meant the region of an antibody that does not comprise the Fc region of the antibody. In accordance with the aforementioned definition of Fc region, “outside the Fc region” for an IgG1 antibody is herein defined to be from the N-terminus up to and including residue T225 or C229, wherein the numbering is according to the EU numbering scheme. Thus the Fab region and part of the hinge region of an antibody are outside the Fc region.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (see review M. in Daeron, Annu Rev. Immunol. 15:203-234 (1997)). FcRs are reviewed in Ravetch and Kinet, Annu Rev. Immunol. 9:457-492 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

An “Fv” fragment is the minimum antibody fragment which comprises the variable domains of its heavy chain and light chain and thus contains a complete antigen recognition and binding site. This region consists of a dimer of one heavy and one light chain variable domain in a tight, non-covalent association (VH-VL dimer). It is in this configuration that the three CDRs of each variable domain interact to define an antigen binding site on the surface of the VH-VL dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site. One or more scFv fragments may be linked to other antibody fragments (such as the constant domain of a heavy chain or a light chain) to form antibody constructs having one or more antigen recognition sites.

The term “single chain variable fragment or scFv” refers to an Fv fragment in which the heavy chain domain and the light chain domain are linked. “Single-chain Fv” also abbreviated as “sFv” or “scFv” are antibody fragments that comprise the VH and VL antibody domains connected into a single polypeptide chain. Generally, the sFv polypeptide further comprises a polypeptide linker between the VH and VL domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

The skilled person will also be familiar with so-called miniantibodies which have a bi-, tri- or tetravalent structure and are derived from scFv. The multimerisation is carried out by di-, tri- or tetrameric coiled coil structures (Pack et al., 1993 Biotechnology II:, 1271-1277; Lovejoy et al. 1993 Science 259: 1288-1293; Pack et al., 1995 J. Mol. Biol. 246: 28-34).

By minibody the skilled person means a bivalent, homodimeric scFv derivative. It consists of a fusion protein which contains the CH3 region of an immunoglobulin, preferably IgG, most preferably IgG1 as the dimerisation region which is connected to the scFv via a hinge region (e.g. also from IgG1) and a linker region. The disulphide bridges in the hinge region are mostly formed in higher cells and not in prokaryotes. In some embodiments an antibody according to the invention is a Notch1-specific minibody antibody fragment. Examples of minibody-antibody proteins from the prior art can be found in Hu et al. (1996, Cancer Res. 56: 3055-61).

The term “diabodies” refers to a small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad Sci. USA 90: 6444-6448 (1993). For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71 and Chapter 6.

By triabody the skilled person means a: trivalent homotrimeric scFv derivative (Kortt et al. 1997 Protein Engineering 10: 423-433). ScFv derivatives wherein VH-VL are fused directly without a linker sequence lead to the formation of trimers.

The Fab fragment [also designated as F(ab)] also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains have a free thiol group. F(ab′) fragments are produced by cleavage of the disulfide bond at the hinge cysteines of the F(ab′)₂ pepsin digestion product. Additional chemical couplings of antibody fragments are known to those of ordinary skill in the art.

A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

An antibody “which binds” an antigen of interest, e.g. a tumor-associated polypeptide antigen target, is one that binds the antigen with sufficient affinity such that the antibody is useful as a diagnostic and/or therapeutic agent in targeting a cell or tissue expressing the antigen, and does not significantly cross-react with other proteins. In such embodiments, the extent of binding of the antibody to a “non-target” protein will be less than about 10% of the binding of the antibody, oligopeptide or other organic molecule to its particular target protein as determined by fluorescence activated cell sorting (FACS) analysis or radioimmunoprecipitation (RIA). With regard to the binding of an antibody to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target.

A “species-dependent antibody,” e.g., a mammalian anti-human Notch1 antibody, is an antibody which has a stronger binding affinity for an antigen from a first mammalian species than it has for a homologue of that antigen from a second mammalian species. Normally, the species-dependent antibody “bind specifically” to a human antigen (i.e., has a binding affinity (Kd) value of no more than about 1×10−7 M, preferably no more than about 1×10−8 and most preferably no more than about 1×10−9 M) but has a binding affinity for homologue of the antigen from a second non-human mammalian species which is at least about 500 fold, or at least about 1000 fold, weaker than its binding affinity for the human antigen. The species-dependent antibody can be of any of the various types of antibodies as defined above, but preferably is a human antibody.

Antibody “effector functions” refer to those biological activities attributable to the Fc region (a native sequence Fc region or amino acid sequence variant Fc region) of an antibody, and vary with the antibody isotype. Examples of antibody effector functions include: Clq binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcyRIII only, whereas monocytes express FcγRI, FcγRII and FcγRIII. FcR expression on hematopoietic cells is summarized in Table 3 on page 464 of Ravetch and Kinet, Annu Rev. Immunol. 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Natl. Acad. Sci. U.S.A. 95:652-656 (1998).

“Complement dependent cytotoxicity” or “CDC” refers to the lysis of a target cell in the presence of complement. Activation of the classical complement pathway is initiated by the binding of the first component of the complement system (Clq) to antibodies (of the appropriate subclass) which are bound to their cognate antigen. To assess complement activation, a CDC assay, e.g., as described in Gazzano-Santoro et al., J. Immunol. Methods 202:163 (1996), may be performed.

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source, e.g., from blood.

The term “cytotoxic agent” as used herein refers to a substance that inhibits or prevents the function of cells and/or causes destruction of cells. The term is intended to include radioactive isotopes (e.g. At 211, I 131, I 125, Y 90, Re 186, Re 188, Sm 153, Bi 212, P 32 and radioactive isotopes of Lu), chemotherapeutic agents e.g. methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal. plant or animal origin, including fragments and/or variants thereof, and the various anti-tumor or anticancer agents disclosed below. Other cytotoxic agents are described below.

A “chemotherapeutic agent” is a chemical compound useful in the treatment of cancer. Examples of chemotherapeutic agents include adriamycin, doxorubicin, epirubicin, 5-fluorouracil, cytosine arabinoside (“Ara-C”), cyclophosphamide, thiotepa, busulfan, cytoxin, taxoids, e.g., paclitaxel (Taxol™, Bristol-Myers Squibb Oncology, Princeton, N.J.), and doxetaxel (Taxotere™, Rhone-Poulenc Rorer, Antony, France), toxotere, methotrexate, cisplatin, melphalan, vinblastine, bleomycin, etoposide, ifosfamide, mitomycin C, mitoxantrone, vincristine, vinorelbine, carboplatin, teniposide, daunomycin, carminomycin, aminopterin, dactinomycin, mitomycins, esperamicins (see U.S. Pat. No. 4,675,187), 6-thioguanine, 6-mercaptopurine, actinomycin D, VP-16, chlorambucil, melphalan, and other related nitrogen mustards. Also included in this definition are hormonal agents that act to regulate or inhibit hormone action on tumors such as tamoxifen and onapristone. A “glycosylation variant” “Glycoform variant” antibody herein is an antibody with one or more carbohydrate moieties attached thereto which differ from one or more carbohydrate moieties attached to a main species antibody. Examples of glycosylation variants herein include antibodies with a G1 or G2 oligosaccharide structure, instead of a GO oligosaccharide structure, attached to an Fc region thereof, antibody with one or two carbohydrate moieties attached to one or two light chains thereof, antibody with no carbohydrate attached to one or two heavy chains of the antibody, etc, as well as combinations of such glycosylation alterations.

“Glycosylation sites” refer to amino acid residues which are recognized by a eukaryotic cell as locations for the attachment of sugar residues. The amino acids where carbohydrate, such as oligosaccharide, is attached are typically asparagine (N-linkage), serine (O-linkage), and threonine (O-linkage) residues. The specific site of attachment is typically signaled by a sequence of amino acids, referred to herein as a “glycosylation site sequence”. The glycosylation site sequence for N-linked glycosylation is: -Asn-X-Ser- or -Asn-X-Thr-, where X may be any of the conventional amino acids, other than proline. The predominant glycosylation site sequence for O-linked glycosylation is: -(Thr or Ser)-X-X-Pro-, where X is any conventional amino acid. The recognition sequence for glycosaminoglycans (a specific type of sulfated sugar) is -Ser-Gly-X-Gly-, where X is any conventional amino acid. The terms“N-linked” and“O-linked” refer to the chemical group that serves as the attachment site between the sugar molecule and the amino acid residue. N-linked sugars are attached through an amino group; O-linked sugars are attached through a hydroxyl group. However, not all glycosylation site sequences in a protein are necessarily glycosylated; some proteins are secreted in both glycosylated and nonglycosylated forms, while others are fully glycosylated at one glycosylation site sequence but contain another glycosylation site sequence that is not glycosylated. Therefore, not all glycosylation site sequences that are present in a polypeptide are necessarily glycosylation sites where sugar residues are actually attached. The initial N-glycosylation during biosynthesis inserts the “core carbohydrate” or “core oligosaccharide” (Proteins, Structures and Molecular Principles, (1984) Creighton (ed.), W.H. Freeman and Company, New York, which is incorporated herein by reference).

A “V region glycosylation site” is a position in a variable region where a carbohydrate, typically an oligosaccharide, is attached to an amino acid residue in the polypeptide chain via an N-linked or O-linked covalent bond. Since not all glycosylation site sequences are necessarily glycosylated in a particular cell, a glycosylation site is defined operationally by reference to a designated cell type in which glycosylation occurs at the site, and is readily determined by one of ordinary skill in the art. Thus, a mutant antibody has at least one mutation that adds, subtracts, or relocates a V region glycosylation site, such as, for example, an N-linked glycosylation site sequence. Preferably, the mutation(s) are substitution mutations that introduce conservative amino acid substitutions, where possible, to modify a glycosylation site. Preferably, when the parent immunoglobulin sequence contains a glycosylation site in a V region framework, particularly in a location near the antigen binding site (for example, near a CDR), the glycosylation site sequence is mutated (e.g., by site-directed mutagenesis) to abolish the glycosylation site sequence, typically by producing a conservative amino acid substitution of one or more of the amino acid residues comprising the glycosylation site sequence. When the parent immunoglobulin sequence contains a glycosylation site in a CDR, and where the parent immunoglobulin specifically binds an epitope that contains carbohydrate, that glycosylation site is preferably retained. If the parent immunoglobulin specifically binds an epitope that comprises only polypeptide, glycosylation sites occuring in a CDR are preferably eliminated by mutation (e.g., site-directed mutation).

“Glycosylation-reduced antibodies” and “glycosylation-reduced immunoglobulin chains” are mutant antibodies and mutant immunoglobulin chains, respectively, in which at least one glycosylation site that is present in the parent sequence has been destroyed by mutation and is absent in the mutant sequence.

“Glycosylation-supplemented antibodies” and “glycosylation-supplemented immunoglobulin chains” are mutant antibodies and mutant immunoglobulin chains, respectively, in which at least one glycosylation site is present in the mutant sequence at a position where no glycosylation site occurs in the parent sequence. Typically, glycosylation-supplemented antibodies that have a higher binding affinity for a carbohydrate-containing epitope than does the parent antibody have a glycosylation site present in a CDR where the parent antibody does not. Typically, a glycosylation-supplemented antibody that specifically binds an epitope that contains polypeptide sequence but no carbohydrate have a lower affinity that the parental antibody.

“Parent immunoglobulin sequence” (or “parent immunoglobulin”) and “parent polynucleotide sequence” refer herein to a reference amino acid sequence or polynucleotide sequence, respectively. A parent polynucleotide sequence may encode a naturally-occurring immunoglobulin chain of a fragment thereof wherein glycosylation site sequences, if any, present in the V region occur about at the same relative amino acid residue position(s) at which glycosylation site sequence(s) are present in naturally-occurring immunoglobulin sequence(s) from which the parent sequence(s) were derived. When mutations, such as site-directed mutations, are introduced into a parent immunoglobulin sequence, the resultant sequence is referred to as a mutant immunoglobulin sequence (or a mutated immunoglobulin sequence).

As used herein, the twenty conventional amino acids and their abbreviations follow conventional usage (Immunology—A Synthesis, 2nd Edition, E. S. Golub and D. R. Gren, Eds., Sinauer Associates, Sunderland, Mass. (1991) which is incorporated herein by reference).

An antibody drug-conjugate (ADCs) is one approach for the treatment of cancer. (Braslawsky et al., Cancer Res, 1990; 50: 6608-14; Liu et al., Proc Natl Acad Sci USA, 1996; 93: 8618-23; Bernstein et al., Leukemia, 2000; 14: 474-5; Ross et al., Cancer Res, 2002; 62: 2546-53; Bhaskar et al., Cancer Res, 2003; 63: 6387-94; Doronina et al., Nat Biotechnol, 2003; 21: 778-84; Francisco et al., Blood, 2003; 102: 1458-65). The strategy of this approach is to deliver a toxic payload to the cancer cell via an antibody that targets a cancer-specific antigen. This strategy requires that the potent drug is internalized via the antibody-antigen complex, released within the cell and specifically kills the cancer cells (Bhaskar et al., Cancer Res, 2003; 63: 6387-94; Doronina et al., Nat Biotechnol, 2003; 21: 778-84; Francisco et al., Blood, 2003; 102: 1458-65). Ideally, the potent drug is internalized via the antibody-antigen complex, released within the cell and specifically kills the cancer cells. In order to minimize toxic side effects it is critical that the molecular target is not expressed in essential organs that are accessible to circulating antibodies. In addition, the target must be at the plasma membrane of cancer cells to allow antibody access.

The same criteria that make a target attractive for an ADC approach to cancer therapy are also desirable for an antibody dependent cellular cytotoxicity (ADCC) approach. In an ADCC approach, a naked antibody to the target is used to recruit immune effector cells (cytotoxic T lymphocytes, natural killer cells, activated macrophages) to the tumor. These effector cells then specifically kill the targeted tumor cells.

As used herein, the term “antibody phage library” refers to the phage library used in the affinity maturation process described above and in Hawkins et at., J. Mol Bio1.254: 889-896 (1992), and in Lowman et al., Biochemistry 30(45): 10832-10838 (1991). Each library comprises a hypervariable region (e.g. 6-7 sites) for which all possible amino acid substitutions are generated. The antibody mutants thus generated are displayed in a monovalent fashion from filamentous phage particles as fusions to the gene III product of M13 packaged within each particle and expressed on the exterior of the phage.

“Single-chain Fv” also abbreviated as “sFv” or “scFv” are antibody fragments that comprise the V H and V L antibody domains connected into a single polypeptide chain. Preferably, the sFv polypeptide further comprises a polypeptide linker between the V H and V L domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra

The term “diabodies” refers to small antibody fragments prepared by constructing sFv fragments (see preceding paragraph) with short linkers (about 5-10 residues) between the V H and V L domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the V H and V L domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

As used herein, “target molecule” means any molecule, not necessarily a protein, for which it is desirable to produce an antibody or ligand. Preferably, however, the target will be a protein and most preferably the target will be an antigen—Notch receptor or human Notch 1 or Notch1 and Notch3.

“Notch” encompasses all members of the Notch receptor family and in particular, Notch1. A “full length” Notch1 receptor protein or nucleic acid refers to a polypeptide or polynucleotide sequence, or a variant thereof, that contains all of the elements normally contained in one or more naturally occurring, wild type Notch1 polynucleotide or polypeptide sequences. For example, a full length Notch1 nucleic acid will typically comprise all of the exons that encode for the full length, naturally occurring protein. The “full length” may be prior to, or after, various stages of post-translation processing.

The term “epitope tagged” when used herein refers to a chimeric polypeptide comprising a polypeptide fused to a “tag polypeptide”. The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

By “solid phase” is meant a non-aqueous matrix to which the antibody of the present invention can adhere. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

A “liposome” is a small vesicle composed of various types of lipids, phospholipids and/or surfactant which is useful for delivery of a drug (such as an IL-17A/F polypeptide or antibody thereto) to a mammal. The components of the liposome are commonly arranged in a bilayer formation, similar to the lipid arrangement of biological membranes.

A “small molecule” is defined herein to have a molecular weight below about 500 Daltons.

The term “prodrug” as used in this application refers to a precursor or derivative of a pharmaceutically active substance that is less cytotoxic to tumor cells compared to the parent drug and is capable of being enzymatically activated or converted into the more active parent form. See, e.g., Wilman, “Prodrugs in Cancer Chemotherapy,” Biochemical Society Transactions, 14, pp. 375-382, 615 Meeting, Belfast (1986) and Stella et al., (ed.), “Prodrugs: A Chemical Approach to Targeted Drug Delivery,” Directed Drug Delivery, Borchardt el al., (ed.), pp. 247-267, Human Press (1985. Examples of cytotoxic drugs that can be derivatized into a prodrug form for use in this invention include, but are not limited to, those chemotherapeutic agents described above.

An “antibody that inhibits the growth of cancer cells expressing Notch receptor or a “growth inhibitory” antibody is one which binds to and results in measurable growth inhibition of cancer cells expressing or overexpressing Notch receptor. Growth inhibition of tumor cells in vivo can be determined in various ways. The antibody is growth inhibitory in vivo if administration of the anti-Notch1 antibody at a therapeutically effective dose results in reduction in tumor size or tumor cell proliferation within a measurable period of time from the first administration of the antibody. Growth inhibition can be measured at an antibody concentration of about 0.1 to 30 μg/ml or about 0.5 nM to 200 nM in cell culture where the growth inhibition is determined 1-10 days after exposure of the tumor cells to the antibody.

The terms “cancer” “neoplasia” and “cancerous” refer to or describe any malignant neoplasm or spontaneous growth or proliferation of cells. The term as used herein encompasses both fully developed malignant neoplasms, as well as premalignant lesions. A subject having “cancer”, for example, may have a tumor.

A “Notch receptor-expressing cancer” is a cancer comprising cells that have Notch receptor protein present on the cell surface. A “Notch receptor-expressing cancer” produces sufficient levels of Notch receptor on the surface of cells thereof, such that a Notch receptor agonist/antagonist or antibody can bind thereto and have a therapeutic effect with respect to the cancer. A cancer which “overexpresses” Notch receptor is one which has significantly higher levels of Notch receptor at the cell surface thereof, compared to a noncancerous cell of the same tissue type. Notch receptor overexpression may be determined in a diagnostic or prognostic assay by evaluating increased levels of the Notch receptor protein present on the surface of a cell (e.g. via an immunohistochemistry assay; FACS analysis). Alternatively, or additionally, one may measure levels of Notch receptor-encoding nucleic acid or mRNA in the cell, e.g. via fluorescent in situ hybridization; (FISH; see WO98/45479 published October, 1998), Southern blotting, Northern blotting, or polymerase chain reaction (PCR) techniques, such as real time quantitative PCR (RT-PCR). One may also study Notch receptor overexpression by measuring shed antigen in a biological fluid such as serum, e.g, using antibody-based assays (see also, e.g., U.S. Pat. No. 4,933,294 issued Jun. 12, 1990; WO91/05264 published Apr. 18, 1991: U.S. Pat. No. 5,401,638 issued Mar. 28, 1995; and Sias et. al. J. Immunol. Methods 132: 73-80 (1990)). Aside from the above assays, various in vivo assays are available to the skilled practitioner. For example, one may expose cells within the body of the patient to an antibody which is optionally labeled with a detectable label, e.g. a radioactive isotope, and binding of the antibody to cells in the patient can be evaluated, e.g. by external scanning for radioactivity or by analyzing a biopsy taken from a patient previously exposed to the antibody.

“Alleviation of cancer” refers to both therapeutic treatment and prophylactic or preventative measures, wherein the object is to prevent or slow down (lessen) the targeted pathologic condition or disorder. Those in need of alleviation include those already with the disorder as well as those prone to have the disorder or those in whom the disorder is to be prevented. A subject or mammal is “alleviated” for a Notch receptor-expressing cancer if, after receiving a therapeutic amount of a Notch receptor agonist according to the methods of the present invention, the patient shows observable and/or measurable reduction in or absence of one or more of the following: reduction in the number of cancer cells or absence of the cancer cells, reduction in the tumor size; inhibition (i.e., slow to some extent and preferably stop) of cancer cell infiltration into peripheral organs; inhibition (i.e., slow to some extent and preferably stop) of tumor metastasis; inhibition, to some extent, of tumor growth; and/or relief to some extent, one or more of the symptoms associated with the specific cancer, and reduced morbidity and mortality. To the extent the Notch receptor agonist/antagonist or antibody may prevent growth and/or kill existing cancer cells, it may be cytostatic and/or cytotoxic. Reduction of these signs or symptoms may also be felt by the patient. Detection and measurement of these above indicators are known to those of skill in the art, including, but not limited for example, reduction in tumor burden, inhibition of tumor size, reduction in proliferation of secondary tumors, expression of genes in tumor tissue, presence of biomarkers, lymph node involvement, histologic grade, and nuclear grade.

The term “therapeutically effective amount” refers to an amount of an agonist and/or antagonist antibody effective to “alleviate” a disease or disorder in a subject or mammal. A “therapeutically effective amount”, in reference to the treatment of tumor, refers to an amount capable of invoking one or more of the following effects: (1) inhibition, to some extent, of tumor growth, including, slowing down and complete growth arrest; (2) reduction in the number of tumor cells; (3) reduction in tumor size; (4) inhibition (i.e., reduction, slowing down or complete stopping) of tumor cell infiltration into peripheral organs; (5) inhibition (i.e., reduction, slowing down or complete stopping) of metastasis; (6) enhancement of anti-tumor immune response, which may, but does not have to, result in the regression or rejection of the tumor; and/or (7) relief, to some extent, of one or more symptoms associated with the disorder. A “therapeutically effective amount” of a Notch1 antibody for purposes of treatment of tumor may be determined empirically and in a routine manner.

The term “inhibition of tumor volume” refers to any decrease or reduction in a tumor volume. The term “tumor volume” refers to the total size of the tumor, which includes the tumor itself plus affected lymph nodes if applicable. Tumor volume may be determined by a variety of methods known in the art, such as, e.g. by measuring the dimensions of the tumor using calipers, computed tomography (CT) or magnetic resonance imaging (MRI) scans, and calculating the volume using equations based on, for example, the z-axis diameter, or on standard shapes such as the sphere, ellipsoid, or cube.

The term “biologically active” (synonymous with “bioactive”) indicates that a composition or compound itself has a biological effect, or that it modifies, causes, promotes, enhances, blocks, reduces, limits the production or activity of, or reacts with or binds to an endogenous molecule that has a biological effect. A “biological effect” may be but is not limited to one that stimulates or causes an immunoreactive response; one that impacts a biological process in an animal; one that impacts a biological process in a pathogen or parasite; one that generates or causes to be generated a detectable signal; and the like. Biologically active compositions, complexes or compounds may be used in therapeutic, prophylactic and diagnostic methods and compositions. Biologically active compositions, complexes or compounds act to cause or stimulate a desired effect upon an animal. Non-limiting examples of desired effects include, for example, preventing, treating or curing a disease or condition in an animal suffering therefrom; limiting the growth of or killing a pathogen in an animal infected thereby; augmenting or altering the phenotype or genotype of an animal; and stimulating a prophylactic immunoreactive response in an animal.

In the context of therapeutic applications of the invention, the term “biologically active” indicates that the composition, complex or compound has an activity that impacts an animal suffering from a disease or disorder in a positive sense and/or impacts a pathogen or parasite in a negative sense. Thus, a biologically active composition, complex or compound may cause or promote a biological or biochemical activity within an animal that is detrimental to the growth and/or maintenance of a pathogen or parasite; or of cells, tissues or organs of an animal that have abnormal growth or biochemical characteristics, such as cancer cells, or cells affected by autoimmune or inflammatory disorders.

In the context of prophylactic applications of the invention, the term “biologically active” indicates that the composition or compound induces or stimulates an immunoreactive response. In some embodiments, the immunoreactive response is designed to be prophylactic, i.e., prevents infection by a pathogen. In other embodiments, the immunoreactive response is designed to cause the immune system of an animal to react to the detriment of cells of an animal, such as cancer cells, that have abnormal growth or biochemical characteristics. In this application of the invention, compositions, complexes or compounds comprising antigens are formulated as a vaccine.

It will be understood by those skilled in the art that a given composition, complex or compound may be biologically active in therapeutic, diagnostic and prophylactic applications. A composition, complex or compound that is described as being “biologically active in a cell” is one that has biological activity in vitro (i.e., in a cell culture) or in vivo (i.e., in the cells of an animal). A “biologically active portion” of a compound or complex is a portion thereof that is biologically active once it is liberated from the compound or complex. It should be noted, however, that such a component may also be biologically active in the context of the compound or complex.

In order to achieve a biological effect, invention constructs may comprise an additional moiety to facilitate internalization and/or uptake by a target cell.

A “patient” or “subject” or “host” refers to either a human or non-human animal.

“Treatment” refers to both therapeutic treatment and prophylactic or preventative measures. Those in need of treatment include those already with the disorder as well as those in which the disorder is to be prevented. The above parameters for assessing successful treatment and improvement in the disease are readily measurable by routine procedures familiar to a physician. For cancer therapy, efficacy can be measured, for example, by assessing the time to disease progression (TTP) and/or determining the response rate (RR). For prostate cancer, the progress of therapy can be assessed by routine methods, usually by measuring serum PSA (prostate specific antigen) levels; the higher the level of PSA in the blood, the more extensive the cancer. Commercial assays for detecting PSA are available, e.g, Hybritech Tandem-E® and Tandem-R® PSA assay kits, the Yang ProsCheck® polyclonal assay (Yang Labs, Bellevue, Wash.), Abbott Imx® (Abbott Labs, Abbott Park, Ill.), etc. Metastasis can be determined by staging tests and by bone scan and tests for calcium level and other enzymes to determine spread to the bone. CT scans can also be done to look for spread to the pelvis and lymph nodes in the area. Chest X-rays and measurement of liver enzyme levels by known methods are used to look for metastasis to the lungs and liver, respectively. Other routine methods for monitoring the disease include transrectal ultrasonography (TRUS) and transrectal needle biopsy (TRNB).

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

An “agonist” or “antagonist” of Notch receptor in addition to binding Notch receptor, has a direct effect on a Notch receptor bearing cell. The Notch receptor agonist will bind Notch receptor, and as well, initiate or mediate the signaling event associated with the Notch receptor, such as, for example, to cause the intracellular domain of Notch to be cleaved and translocated to the nucleus. Here it induces the synthesis of transcriptional repressors members of the Hes family as for example HES-1. When ECD truncated forms of Notch are tested, the HES-1 promoter is strongly stimulated. Activation of the HES-1 promoter can be assayed in vitro. The ability to induce Notch receptor activation can be quantified using techniques known in the art such as reporter constructs such as Beta-galactosidase, chloramphenicol acetyl transferase (CAT) or luciferase. The Notch receptor antagonist will inhibit signaling transmitted from the Notch receptor through various mechanisms including blocking cleavage of the intracellular domain or stimulating receptor degradation.

The Notch receptor “antagonist” will inhibit signaling transmitted from the Notch receptor through various mechanisms including blocking cleavage of the intracellular domain, blocking binding of the endogenous ligand to the receptor or stimulating receptor degradation. The term “antagonist” is used in the broadest sense, and includes any molecule that partially or fully blocks, inhibits, or neutralizes a biological activity of a native Notch receptor protein.

Suitable agonist or antagonist molecules specifically include agonist or antagonist antibodies or antibody fragments, fragments or amino acid sequence variants thereof etc. Methods for identifying agonists or antagonists of a Notch receptor polypeptide are known in the art. An exemplary method proposes contacting a Notch1 bearing cells or tissue with a candidate agonist or antagonist molecule and measuring a detectable change in one or more biological activities normally associated with the Notch receptor.

Additional downstream targets of the Notch signal transduction pathway may be used as biomarkers to evaluate the effects of Notch receptor agonists or antagonists. Recent work suggests that expression of the HESS gene, which encodes an E(spl)-like protein, depends on an intact Notch signaling pathway (Davis and Turner, 2001, Oncogene, 20, 8342-8357). HES5 expression is reduced or abolished in mice mutant for Notch1, RBP-Jx, or presenilin1 and 2 (presenilins are required for Notch proteolytic activation) (de la Pompa et al., 1997, Development, 124, 1139-1148). Notch signaling has also been observed to regulate expression of the Hey genes in mice. Expression of the Hey genes in mouse embryos depends on function of the Notch ligand Dll-1 (Kokubo et al., 1999, Biochem. Biophys. Res. Commun., 260, 459-465). Consistent with this, the promoter regions of the three mammalian Hey genes contain binding sites for CSL proteins, and these promoters are responsive in cultured cells to activated Notch signaling (Maier and Gessler, 2000, Biochem. Biophys. Res. Commun., 275, 652-660). Transgenic expression of activated Notch in mouse hair follicles leads to ectopic expression of the HeyL gene, while mutant mice for Notch 1 or the Notch ligand Dll-1 lose expression of HeyL in the presomitic mesoderm (Lin et al., 2000, Development, 127, 2421-2432).

“Mammal” for purposes of treatment refers to any animal classified as a mammal, including humans, domestic and farm animals, and zoo, sports, or pet animals, such as dogs, cats, cattle, horses, sheep, pigs, goats, rabbits, etc. Preferably, the mammal is human.

The term “administering” includes any method of delivery of a compound of the present invention, including but not limited to, a pharmaceutical composition or therapeutic agent, into a subject's system or to a particular region in or on a subject. The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration. “Parenteral administration” and “administered parenterally” means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

Administration “in combination with” one or more further therapeutic agents includes simultaneous (concurrent) and consecutive administration in any order.

The term “modulate” means to affect (e.g., either upregulate, downregulate or otherwise control) the level of a signaling pathway. Cellular processes under the control of signal transduction include, but are not limited to, transcription of specific genes, normal cellular functions, such as metabolism, proliferation, differentiation, adhesion, apoptosis and survival, as well as abnormal processes, such as transformation, blocking of differentiation and metastasis.

A “disorder” is any condition that would benefit from treatment with the polypeptide. This includes chronic and acute disorders or diseases including those pathological conditions which predispose the mammal to the disorder in question.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

Completely “human” antibodies may be desirable for therapeutic treatment of human patients. Human antibodies can be made by a variety of methods known in the art including phage display methods described above using antibody libraries derived from human immunoglobulin sequences. See U.S. Pat. Nos. 4,444,887 and 4,716,111; and PCT publications WO 98/46645; WO 98/50433; WO 98/24893; WO 98/16654; WO 96/34096; WO 96/33735; and WO 91/10741, each of which is incorporated herein by reference in its entirety. Human antibodies can also be produced using transgenic mice which are incapable of expressing functional endogenous immunoglobulins, but which can express human immunoglobulin genes, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entireties. In addition, companies such as Abgenix, Inc. (Fremont, Calif.) and Medarex (Princeton, N.J.) can be engaged to provide human antibodies directed against a selected antigen using technology similar to that described above.

Completely human antibodies that recognize a selected epitope can be generated using a technique referred to as “guided selection.” In this approach a selected non-human monoclonal antibody, e.g., a mouse antibody, is used to guide the selection of a completely human antibody recognizing the same epitope (Jespers et al., Biotechnology 12:899-903 (1988).

An “effector” or “effector moiety” or “effector component” is a molecule that is bound (or linked, or conjugated), either covalently, through a linker or a chemical bond, or noncovalently, through ionic, van der Waals, electrostatic, or hydrogen bonds, to an antibody. The “effector” can be a variety of molecules including, e.g., detection moieties including radioactive compounds, fluorescent compounds, an enzyme or substrate, tags such as epitope tags, a toxin, activatable moieties, a chemotherapeutic or cytotoxic agent, a chemoattractant, a lipase; an antibiotic; or a radioisotope emitting “hard” e.g., beta radiation.

The same criteria that make a target attractive for an ADC approach to cancer therapy are also desirable for an antibody dependent cellular cytotoxicity (ADCC) approach. In an ADCC approach, a naked antibody to the target is used to recruit immune effector cells (cytotoxic T lymphocytes, natural killer cells, activated macrophages) to the tumor. These effector cells then specifically kill the targeted tumor cells.

“Biological sample” as used herein is a sample of biological tissue or cells that contains nucleic acids or polypeptides, e.g., Notch1 or Notch3 protein, polynucleotide or transcript. Such samples include, but are not limited to, tissue isolated from primates (e.g., humans) or from rodents (e.g., mice, and rats). Biological samples may also include sections of tissues such as biopsy and autopsy samples, frozen sections taken for histologic purposes, skin, etc. Biological samples also include explants and primary and/or transformed cell cultures derived from patient tissues. A biological sample is typically obtained from a eukaryotic organism, most preferably a mammal such as a primate e.g., human.

“Providing a biological sample” means to obtain a biological sample for use in methods described in this invention. Most often, this will be done by removing a sample of cells from a human, but can also be accomplished by using previously isolated cells (e.g., isolated by another person, at another time, and/or for another purpose), or by performing the methods of the invention in vivo.

“Tumor”, as used herein, refers to all neoplastic cell growth and proliferation, whether malignant or benign, and all pre-cancerous and cancerous cells and tissues.

“Notch” encompasses all members of the Notch receptor family and in particular, Notch1 and Notch3.

The Notch ligands include Jagged1, Jagged2, Delta1, Delta3, and Delta4. “Notch1” cDNA and deduced amino acid sequence is as set forth in SEQ ID NO:1 and SEQ ID NO:2, respectively.

A “label” or a “detectable moiety” is a composition detectable by spectroscopic, photochemical, biochemical, immunochemical, chemical, or other physical means. The word “label” when used herein refers to a detectable compound or composition which is conjugated directly or indirectly to the antibody so as to generate a “labeled” antibody. The label may be detectable by itself (e.g. radioisotope labels or fluorescent labels) or, in the case of an enzymatic label, may catalyze chemical alteration of a substrate compound or composition which is detectable. For example, useful labels include fluorescent dyes, electron-dense reagents, enzymes (e.g., as commonly used in an ELISA), biotin, digoxigenin, colloidal gold, luminescent nanocrystals (e.g. quantum dots), haptens and proteins or other entities which can be made detectable, e.g., by incorporating a radiolabel into the peptide or used to detect antibodies specifically reactive with the peptide. The radioisotope may be, for example, ³H, ¹⁴C, ³²P, ³⁵S, or ¹²⁵I. In some cases, particularly using antibodies against the proteins of the invention, the radioisotopes are used as toxic moieties, as described below. Any method known in the art for conjugating the antibody to the label may be employed. The lifetime of radiolabeled peptides or radiolabeled antibody compositions may be extended by the addition of substances that stabilize the radiolabeled peptide or antibody and protect it from degradation. Any substance or combination of substances that stabilize the radiolabeled antibody may be used including those substances disclosed in U.S. Pat. No. 5,961,955, which is incorporated by reference herein in its entirety.

### Antibody Structure

Naturally occurring (wildtype) antibody molecules are Y-shaped molecules consisting of four polypeptide chains, two identical heavy chains and two identical light chains, which are covalently linked together by disulfide bonds. Both types of polypeptide chains have constant regions, which do not vary or vary minimally among antibodies of the same class (i.e., IgA, IgM, etc.), and variable regions. The variable regions are unique to a particular antibody and comprise a recognition element for an epitope. The carboxy-terminal regions of both heavy and light chains are conserved in sequence and are called the constant regions (also known as C-domains). The amino-terminal regions (also known as V-domains) are variable in sequence and are responsible for antibody specificity. The antibody specifically recognizes and binds to an antigen mainly through six short complementarity-determining regions (CDRs) located in their V-domains.

Each light chain of an antibody is associated with one heavy chain, and the two chains are linked by a disulfide bridge formed between cysteine residues in the carboxy-terminal region of each chain, which is distal from the amino terminal region of each chain that constitutes its portion of the antigen binding domain. Antibody molecules are further stabilized by disulfide bridges between the two heavy chains in an area known as the hinge region, at locations nearer the carboxy terminus of the heavy chains than the locations where the disulfide bridges between the heavy and light chains are made. The hinge region also provides flexibility for the antigen-binding portions of an antibody.

An antibody's specificity is determined by the variable regions located in the amino terminal regions of the light and heavy chains. The variable regions of a light chain and associated heavy chain form an “antigen binding domain” that recognizes a specific epitope; an antibody thus has two antigen binding domains. The antigen binding domains in a wild type antibody are directed to the same epitope of an immunogenic protein, and a single wild type antibody is thus capable of binding two molecules of the immunogenic protein at the same time. Thus, a wild type antibody is monospecific (i.e., directed to a unique antigen) and divalent (i.e., capable of binding two molecules of antigen).

### Types of Antibodies

“Polyclonal antibodies” are generated in an immunogenic response to a protein having many epitopes. A composition (e.g., serum) of polyclonal antibodies thus includes a variety of different antibodies directed to the same and to different epitopes within the protein. Methods for producing polyclonal antibodies are known in the art (see, e.g., Cooper et al., Section III of Chapter 11 in: Short Protocols in Molecular Biology, 2nd Ed., Ausubel et al., eds., John Wiley and Sons, New York, 1992, pages 11-37 to 11-41).

“Antipeptide antibodies” (also known as “monospecific antibodies”) are generated in a humoral response to a short (typically, 5 to 20 amino acids) immunogenic polypeptide that corresponds to a few (preferably one) isolated epitopes of the protein from which it is derived. A plurality of antipeptide antibodies includes a variety of different antibodies directed to a specific portion of the protein, i.e., to an amino acid sequence that contains at least one, preferably only one, epitope. Methods for producing antipeptide antibodies are known in the art (see, e.g., Cooper et al., Section III of Chapter 11 in: Short Protocols in Molecular Biology, 2nd Ed., Ausubel et al., eds., John Wiley and Sons, New York, 1992, pages 11-42 to 11-46).

A “Monoclonal antibody” is a specific antibody that recognizes a single specific epitope of an immunogenic protein. In a plurality of a monoclonal antibody, each antibody molecule is identical to the others in the plurality. In order to isolate a monoclonal antibody, a clonal cell line that expresses, displays and/or secretes a particular monoclonal antibody is first identified; this clonal cell line can be used in one method of producing the antibodies of the invention. Methods for the preparation of clonal cell lines and of monoclonal antibodies expressed thereby are known in the art (see, for example, Fuller et al., Section II of Chapter 11 in: Short Protocols in Molecular Biology, 2nd Ed., Ausubel et al., eds., John Wiley and Sons, New York, 1992, pages 11-22 to 11-11-36).

A “Naked antibody” is an antibody that lacks the Fc portion of a wildtype antibody molecule. The Fc portion of the antibody molecule provides effector functions, such as complement fixation and ADCC (antibody dependent cell cytotoxicity), which set mechanisms into action that may result in cell lysis. See, e.g., Markrides, Therapeutic inhibition of the complement system, Pharmacol. Rev. 50:59-87, 1998. In some systems, it appears that the therapeutic action of an antibody depends upon the effector functions of the Fc region (see, e.g., Golay et al., Biologic response of B lymphoma cells to anti-CD20 monoclonal antibody rituximab in vitro: CD55 and CD59 regulate complement-mediated cell lysis, Blood 95:3900-3908, 2000).

However, it is possible that the Fc portion is not required for therapeutic function in every instance, as other mechanisms, such as apoptosis, can come into play. Moreover, the Fc region may be deleterious in some applications as antibodies comprising an Fc region are taken up by Fc receptor-bearing cells, thereby reducing the amount of therapeutic antibody taken up by targeted cells. Vaswani and Hamilton, Humanized antibodies as potential therapeutic drugs, Ann. Allergy Asthma Immunol. 81:105-119, 1998. Components of the immune system may recognize and react to antibodies that are clumped together on the surface of tumor cells. It is thus envisioned that the resulting immune response will target and destroy, or at least limit the proliferation of, the tumor cells.

One way to get naked antibodies delivered to surfaces where they will clump together is to use a targetable construct or complex to bring different naked antibodies together on a targeted cellular surface. By way of non-limiting example, an anti-C20 antibody (e.g., Rituxan) and an anti-C22 antibody might be administered separately or together, allowed to clear so that unbound antibodies are removed from the system.

Naked antibodies are also of interest for therapy of diseases caused by parasites, such as malaria. Vukovic et al., Immunoglobulin G3 antibodies specific for the 19-kilodalton carboxyl-terminal fragment of Plasmodium yoelii merozoite surface protein 1 transfer protection to mice deficient in Fc-RI receptors, Infect. Immun. 68:3019-22, 2000.

“Single chain antibodies (scFv)” generally do not include portions of the Fc region of antibodies that are involved in effector functions and are thus naked antibodies, although methods are known for adding such regions to known scFv molecules if desired. See Helfrich et al., A rapid and versatile method for harnessing scFv antibody fragments with various biological functions, J. Immunol. Meth. 237:131-145,2000; and de Haard et al., Creating and engineering human antibodies for immunotherapy, Adv. Drug Delivery Rev. 31:5-31, 1998.

### Antibody Fragments

### Proteolytic Antibody Fragments

Antibody fragments produced by limited proteolysis of wild type antibodies are called proteolytic antibody fragments. These include, but are not limited to, the following.

“F(ab′)₂ fragments” are released from an antibody by limited exposure of the antibody to a proteolytic enzyme, e.g., pepsin or ficin. An F(ab′)₂ fragment comprises two “arms,” each of which comprises a variable region that is directed to and specifically binds a common antigen. The two Fab′ molecules are joined by interchain disulfide bonds in the hinge regions of the heavy chains; the Fab′ molecules may be directed toward the same (bivalent) or different (bispecific) epitopes.

“Fab′ fragments” contain a single anti-binding domain comprising an Fab and an additional portion of the heavy chain through the hinge region.

“Fab′-SH fragments” are typically produced from F(ab′)₂ fragments, which are held together by disulfide bond(s) between the H chains in an F(ab′)₂ fragment. Treatment with a mild reducing agent such as, by way of non-limiting example, beta-mercaptoethylamine, breaks the disulfide bond(s), and two Fab′ fragments are released from one F(ab′)₂ fragment. Fab′-SH fragments are monovalent and monospecific.

“Fab fragments” (i.e., an antibody fragment that contains the antigen-binding domain and comprises a light chain and part of a heavy chain bridged by a disulfide bond) are produced by papain digestion of intact antibodies. A convenient method is to use papain immobilized on a resin so that the enzyme can be easily removed and the digestion terminated. Fab fragments do not have the disulfide bond(s) between the H chains present in an F(ab′)₂ fragment.

### Recombinant Antibody Fragments

“Single-chain antibodies” are one type of antibody fragment. The term single chain antibody is often abbreviated as “scFv” or “sFv.” These antibody fragments are produced using molecular genetics and recombinant DNA technology. A single-chain antibody consists of a polypeptide chain that comprises both a V_{H} and a V_{L} portion. Unlike wildtype antibodies, wherein two separate heavy and light polypeptide chains are conjoined to form a single antigen-binding variable region, a single-chain antibody is a single polypeptide that comprises an antigen-binding variable region. That is, a single-chain antibody comprises the variable, antigen-binding determinative region of a single light and heavy chain of an antibody linked together by a chain of 10-25 amino acids.

The term “single-chain antibody” includes but is not limited to a disulfide-linked Fv (dsFv) in which two single-chain antibodies linked together by a disulfide bond; a bispecific sFv (a sFv or a dsFv molecule having two antigen-binding domains, each of which may be directed to a different epitope); a diabody (a dimerized sFv formed when the V_{H} domain of a first sFv assembles with the V_{L} domain of a second sFv and the V_{L} domain of the first sFv assembles with the V_{H} domain of the second sFv; the two antigen-binding regions of the diabody may be directed towards the same or different epitopes); and a triabody (a trimerized sFv, formed in a manner similar to a diabody, but in which three antigen-binding domains are created in a single complex; the three antigen binding domains may be directed towards the same or different epitopes).

“Fully human antibodies” are human antibodies that can be produced in transgenic animals such as XenoMice™. XenoMouse™ strains are genetically engineered mice in which the murine IgH and Igk loci have been functionally replaced by their human Ig counterparts on yeast artificial YAC transgenes. These human Ig transgenes can carry the majority of the human variable repertoire and can undergo class switching from IgM to IgG isotypes. The immune system of the XenoMouse™ recognizes administered human antigens as foreign and produces a strong humoral response. The use of XenoMouse™ in conjunction with well-established hybridomas techniques, results in fully human IgG mAbs with sub-nanomolar affinities for human antigens (see U.S. Pat. No. 5,770,429, entitled “Transgenic non-human animals capable of producing heterologous antibodies”, U.S. Pat. No. 6,162,963, entitled “Generation of xenogenetic antibodies”; U.S. Pat. No. 6,150,584, entitled “Human antibodies derived from immunized XenoMice™”, U.S. Pat. No. 6,114,598, entitled “Generation of xenogeneic antibodies”; and U.S. Pat. No. 6,075,181, entitled “Human antibodies derived from immunized XenoMice™”; for reviews, see Green, Antibody engineering via genetic engineering of the mouse: XenoMouse™ strains are a vehicle for the facile generation of therapeutic human monoclonal antibodies, J. Immunol. Meth. 231:11-23, 1999; Wells, Eek, a XenoMouse™: Abgenix, Inc., Chem. Biol. 7:R185-6, 2000; and Davis et al., Transgenic mice as a source of filly human antibodies for the treatment of cancer, Cancer Metastasis Rev. 18:421-5, 1999).

“Complementary determining region peptides” or “CDR peptides” are another form of an antibody fragment. A CDR peptide (also known as “minimal recognition unit”) is a peptide corresponding to a single complementarity-determining region (CDR), and can be prepared by constructing genes encoding the CDR of an antibody of interest. Such genes are prepared, for example, by using the polymerase chain reaction to synthesize the variable region from RNA of antibody-producing cells. See, for example, Larrick et al., Methods: A Companion to Methods in Enzymology 2:106, 1991.

Compositions of the Invention and Methods of making same

This invention encompasses encompasses a plurality of substantially pure, isolated anti-Notch1 isolated antibodies and polynucleotide embodiments. Explicitly included are compositions, including pharmaceutical compositions, comprising an anti-Notch1 antibody; and polynucleotides comprising sequences encoding an anti-Notch1 antibody. As used herein, compositions comprise one or more antibodies that bind to Notch1, and/or one or more polynucleotides comprising sequences encoding one or more antibodies that bind to Notch1. These compositions may further comprise suitable carriers, such as pharmaceutically acceptable excipients including buffers, which are well known in the art.

The anti-Notch1 antibodies of the invention are preferably monoclonal. Also encompassed within the scope of the invention are Fab, Fab′, Fab′-SH and F(ab′)₂ fragments of the anti-Notch1 antibodies provided herein. Single chain anti-Notch 1 antibodies as well as multispecific and multivariant Notch1 specific antibodies are also included. These antibody fragments can be created by traditional means, such as enzymatic digestion, or may be generated by recombinant techniques. These fragments are useful for the diagnostic and therapeutic purposes set forth below.

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies.

The anti-Notch1 monoclonal antibodies of the invention are preferably made by recombinant DNA methods (U.S. Pat. No. 4,816,567).

The binding specificity of monoclonal antibodies produced by recombinant means is determined by immunoprecipitation or by an in vitro binding assay, such as radioimmunoassay (RIA) or enzyme-linked immunoadsorbent assay (ELISA).

The binding affinity of the monoclonal antibody can, for example, be determined by the Scatchard analysis of Munson et al., Anal. Biochem., 107:220 (1980).

The anti-Notch1 antibodies of the invention can be made by using combinatorial libraries to screen for synthetic antibody clones with the desired activity or activities. In principle, synthetic antibody clones are selected by screening phage libraries containing phage that display various fragments of antibody variable region (Fv) fused to phage coat protein. Such phage libraries are panned by affinity chromatography against the desired antigen. Clones expressing Fv fragments capable of binding to the desired antigen are adsorbed to the antigen and thus separated from the non-binding clones in the library. The binding clones are then eluted from the antigen, and can be further enriched by additional cycles of antigen adsorption/elution. Any of the anti-Notch1 antibodies of the invention can be obtained by designing a suitable antigen screening procedure to select for the phage clone of interest followed by construction of a full length anti-Notch1 antibody clone using the Fv sequences from the phage clone of interest and suitable constant region (Fc) sequences described in Kabat et al., Sequences of Proteins of Immunological Interest, Fifth Edition, NIH Publication 91-3242, Bethesda Md. (1991), vols. 1-3.

The antigen-binding domain of an antibody is formed from two variable (V) regions of about 110 amino acids, one each from the light (VL) and heavy (VH) chains, that both present three hypervariable loops or complementarity-determining regions (CDRs). Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described in Winter et al., Ann. Rev. Immunol., 12: 433-455 (1994). As used herein, scFv encoding phage clones and Fab encoding phage clones are collectively referred to as “Fv phage clones” or “Fv clones”.

Repertoires of VH and VL genes can be separately cloned by polymerase chain reaction (PCR) and recombined randomly in phage libraries, which can then be searched for antigen-binding clones as described in Winter et al., Ann. Rev. Immunol., 12: 433-455 (1994). Libraries from immunized sources provide high-affinity antibodies to the immunogen without the requirement of constructing hybridomas. Alternatively, the naive repertoire can be cloned to provide a single source of human antibodies to a wide range of non-self and also self antigens without any immunization as described by Griffiths et al., EMBO J. 12: 725-734 (1993). Finally, naive libraries can also be made synthetically by cloning the unrearranged V-gene segments from stem cells, and using PCR primers containing random sequence to encode the highly variable CDR3 regions and to accomplish rearrangement in vitro as described by Hoogenboom and Winter, J. Mol. Biol., 227: 381-388 (1992).

Filamentous phage is used to display antibody fragments by fusion to the minor coat protein pIII. The antibody fragments can be displayed as single chain Fv fragments, in which VH and VL domains are connected on the same polypeptide chain by a flexible polypeptide spacer, e.g. as described by Marks et al., J. Mol. Biol., 222: 581-597 (1991), or as Fab fragments, as described in Hoogenboom et al., Nucl. Acids Res., 19: 4133-4137 (1991).

In general, nucleic acids encoding antibody gene fragments are obtained from immune cells harvested from humans. If a library biased in favor of anti-Notch1 clones is desired, the subject is immunized with Notch1 to generate an antibody response, and spleen cells and/or circulating B cells other peripheral blood lymphocytes (PBLs) are recovered for library construction. In another embodiment, a human antibody gene fragment library biased in favor of anti-Notch1 clones is obtained by generating an anti-Notch1 antibody response in transgenic mice carrying a functional human immunoglobulin gene array (and lacking a functional endogenous antibody production system) such that Notch1 immunization gives rise to B cells producing human antibodies against Notch1. The generation of human antibody-producing transgenic mice is described below.

Additional enrichment for anti-Notch1 reactive cell populations can be obtained by using a suitable screening procedure to isolate B cells expressing Notch1-specific membrane bound antibody, e.g., by cell separation with Notch1 affinity chromatography or adsorption of cells to fluorochrome-labeled Notch1 followed by flow-activated cell sorting (FACS).

Alternatively, the use of spleen cells and/or B cells or other PBLs from an unimmunized donor provides a better representation of the possible antibody repertoire, and also permits the construction of an antibody library using any animal (human or non-human) species in which Notch1 is not antigenic. For libraries incorporating in vitro antibody gene construction, stem cells are harvested from the subject to provide nucleic acids encoding unrearranged antibody gene segments. The immune cells of interest can be obtained from a variety of animal species, such as human, mouse, rat, lagomorpha, luprine, canine, feline, porcine, bovine, equine, and avian species, etc.

Nucleic acid encoding antibody variable gene segments (including VH and VL segments) are recovered from the cells of interest and amplified. In the case of rearranged VH and VL gene libraries, the desired DNA can be obtained by isolating genomic DNA or mRNA from lymphocytes followed by polymerase chain reaction (PCR) with primers matching the 5′ and 3′ ends of rearranged VH and VL genes as described in Orlandi et al., Proc. Natl. Acad. Sci. (USA), 86: 3833-3837 (1989), thereby making diverse V gene repertoires for expression. The V genes can be amplified from cDNA and genomic DNA, with back primers at the 5′ end of the exon encoding the mature V-domain and forward primers based within the J-segment as described in Orlandi et al. (1989) and in Ward et al., Nature, 341: 544-546 (1989). However, for amplifying from cDNA, back primers can also be based in the leader exon as described in Jones et al., Biotechnol., 9: 88-89 (1991), and forward primers within the constant region as described in Sastry et al., Proc. Natl. Acad. Sci. (USA), 86: 5728-5732 (1989). To maximize complementarity, degeneracy can be incorporated in the primers as described in Orlandi et al. (1989) or Sastry et al. (1989). Preferably, the library diversity is maximized by using PCR primers targeted to each V-gene family in order to amplify all available VH and VL arrangements present in the immune cell nucleic acid sample, e.g. as described in the method of Marks et al., J. Mol. Biol., 222: 581-597 (1991) or as described in the method of Orum et al., Nucleic Acids Res., 21: 4491-4498 (1993). For cloning of the amplified DNA into expression vectors, rare restriction sites can be introduced within the PCR primer as a tag at one end as described in Orlandi et al. (1989), or by further PCR amplification with a tagged primer as described in Clackson et al., Nature, 352: 624-628 (1991).

Repertoires of synthetically rearranged V genes can be derived in vitro from V gene segments. Most of the human VH-gene segments have been cloned and sequenced (reported in Tomlinson et al., J. Mol. Biol., 227: 776-798 (1992)), and mapped (reported in Matsuda et al., Nature Genet., 3: 88-94 (1993); these cloned segments (including all the major conformations of the H1 and H2 loop) can be used to generate diverse VH gene repertoires with PCR primers encoding H3 loops of diverse sequence and length as described in Hoogenboom and Winter, J. Mol. Biol., 227: 381-388 (1992). VH repertoires can also be made with all the sequence diversity focused in a long H3 loop of a single length as described in Barbas et al., Proc. Natl. Acad. Sci. USA, 89: 4457-4461 (1992). Human V.kappa. and Vλ segments have been cloned and sequenced (reported in Williams and Winter, Eur. J. Immunol., 23: 1456-1461 (1993)) and can be used to make synthetic light chain repertoires. Synthetic V gene repertoires, based on a range of VH and VL folds, and L3 and H3 lengths, will encode antibodies of considerable structural diversity. Following amplification of V-gene encoding DNAs, germline V-gene segments can be rearranged in vitro according to the methods of Hoogenboom and Winter, J. Mol. Biol., 227: 381-388 (1992).

Repertoires of antibody fragments can be constructed by combining VH and VL gene repertoires together in several ways. Each repertoire can be created in different vectors, and the vectors recombined in vitro, e.g., as described in Hogrefe et al., Gene, 128: 119-126 (1993), or in vivo by combinatorial infection, e.g., the loxP system described in Waterhouse et al., Nucl. Acids Res., 21: 2265-2266 (1993). The in vivo recombination approach exploits the two-chain nature of Fab fragments to overcome the limit on library size imposed by E. coli transformation efficiency. Naive VH and VL repertoires are cloned separately, one into a phagemid and the other into a phage vector. The two libraries are then combined by phage infection of phagemid-containing bacteria so that each cell contains a different combination and the library size is limited only by the number of cells present (about 10¹² clones). Both vectors contain in vivo recombination signals so that the VH and VL genes are recombined onto a single replicon and are co-packaged into phage virions. These huge libraries provide large numbers of diverse antibodies of good affinity (K_{d}⁻¹ of about 10^{-8 M} ).

Alternatively, the repertoires may be cloned sequentially into the same vector, e.g. as described in Barbas et al., Proc. Natl. Acad. Sci. USA, 88: 7978-7982 (1991), or assembled together by PCR and then cloned, e.g. as described in Clackson et al., Nature, 352: 624-628 (1991). PCR assembly can also be used to join VH and VL DNAs with DNA encoding a flexible peptide spacer to form single chain Fv (scFv) repertoires. In yet another technique, “in cell PCR assembly” is used to combine VH and VL genes within lymphocytes by PCR and then clone repertoires of linked genes as described in Embleton et al., Nucl. Acids Res., 20: 3831-3837 (1992).

The antibodies produced by naive libraries (either natural or synthetic) can be of moderate affinity (K_{d}⁻¹ of about 10⁶ to 10⁷ M⁻¹), but affinity maturation can also be mimicked in vitro by constructing and reselecting from secondary libraries as described in Winter et al. (1994), supra. For example, mutation can be introduced at random in vitro by using error-prone polymerase (reported in Leung et al., Technique, 1: 11-15 (1989)) in the method of Hawkins et al., J. Mol. Biol., 226: 889-896 (1992) or in the method of Gram et al., Proc. Natl. Acad. Sci. USA, 89: 3576-3580 (1992). Additionally, affinity maturation can be performed by randomly mutating one or more CDRs, e.g. using PCR with primers carrying random sequence spanning the CDR of interest, in selected individual Fv clones and screening for higher affinity clones. WO 9607754 (published 14 Mar. 1996) described a method for inducing mutagenesis in a complementarity determining region of an immunoglobulin light chain to create a library of light chain genes. Another effective approach is to recombine the VH or VL domains selected by phage display with repertoires of naturally occurring V domain variants obtained from unimmunized donors and screen for higher affinity in several rounds of chain reshuffling as described in Marks et al., Biotechnol., 10: 779-783 (1992). This technique allows the production of antibodies and antibody fragments with affinities in the 10⁻⁹ M range.

Notch1 nucleic acid and amino acid sequences are known in the art. A representative nucleic acid and amino acid sequence of Notch1 is detailed in SEQ ID NOS. 1 and 2 respectively. Nucleic acid sequence encoding the Notch1 can be designed using the amino acid sequence of the desired region of Notch1. Alternatively, the cDNA sequence (or fragments thereof) of GenBank Accession Nos. NM₋₀₁₉₀₇₄. Notch1 is a transmembrane protein. The extracellular region contains 36 EGF-like repeats, as well as a DSL domain that is conserved among all Notch ligands and is necessary for receptor binding. The predicted protein also contains a transmembrane region, and a cytoplasmic tail lacking any catalytic motifs. Human Notch1 protein is a 685 amino acid protein. The accession number of human Notch1 is NM₋₋₀₁₉₀₇₄. See Sarah J. Bray, “Notch signaling: a simple pathway becomes complex” Nature Reviews Molecular Cell Biology, 7: 678-689 (2006), the entire content of which is incorporated by reference herein.

DNAs encoding Notch1 can be prepared by a variety of methods known in the art. These methods include, but are not limited to, chemical synthesis by any of the methods described in Engels et al., Agnew. Chem. Int Ed. Engl., 28: 716-734 (1989), such as the triester, phosphite, phosphoramidite and H-phosphonate methods. In one embodiment, codons preferred by the expression host cell are used in the design of the Notch1 encoding DNA. Alternatively, DNA encoding the Notch1 can be isolated from a genomic or cDNA library.

Following construction of the DNA molecule encoding the Notch1, the DNA molecule is operably linked to an expression control sequence in an expression vector, such as a plasmid, wherein the control sequence is recognized by a host cell transformed with the vector. In general, plasmid vectors contain replication and control sequences which are derived from species compatible with the host cell. The vector ordinarily carries a replication site, as well as sequences which encode proteins that are capable of providing phenotypic selection in transformed cells. Suitable vectors for expression in prokaryotic and eukaryotic host cells are known in the art and some are further described herein. Eukaryotic organisms, such as yeasts, or cells derived from multicellular organisms, such as mammals, may be used.

Optionally, the DNA encoding the Notch1 is operably linked to a secretory leader sequence resulting in secretion of the expression product by the host cell into the culture medium. Examples of secretory leader sequences include stII, ecotin, lamB, herpes GD, lpp, alkaline phosphatase, invertase, and alpha factor. Also suitable for use herein is the 36 amino acid leader sequence of protein A (Abrahmsen et al., EMBO J., 4: 3901 (1985)).

Host cells are transfected and preferably transformed with the above-described expression or cloning vectors of this invention and cultured in conventional nutrient media modified as appropriate for inducing promoters, selecting transformants, or amplifying the genes encoding the desired sequences.

Transfection refers to the taking up of an expression vector by a host cell whether or not any coding sequences are in fact expressed. Numerous methods of transfection are known to the ordinarily skilled artisan, for example, CaPO₄ precipitation and electroporation. Successful transfection is generally recognized when any indication of the operation of this vector occurs within the host cell. Methods for transfection are well known in the art, and some are further described herein.

Transformation means introducing DNA into an organism so that the DNA is replicable, either as an extrachromosomal element or by chromosomal integrant. Depending on the host cell used, transformation is done using standard techniques appropriate to such cells. Methods for transformation are well known in the art, and some are further described herein.

Prokaryotic host cells used to produce the Notch1 can be cultured as described generally in Sambrook et al., supra.

The mammalian host cells used to produce the Notch1 can be cultured in a variety of media, which is well known in the art and some of which is described herein.

The host cells referred to in this disclosure encompass cells in in vitro culture as well as cells that are within a host animal.

Purification of Notch1 may be accomplished using art-recognized methods, some of which are described herein.

The purified Notch1 can be attached to a suitable matrix such as agarose beads, acrylamide beads, glass beads, cellulose, various acrylic copolymers, hydroxyl methacrylate gels, polyacrylic and polymethacrylic copolymers, nylon, neutral and ionic carriers, and the like, for use in the affinity chromatographic separation of phage display clones. Attachment of the Notch1 protein to the matrix can be accomplished by the methods described in Methods in Enzymology, vol. 44 (1976). A commonly employed technique for attaching protein ligands to polysaccharide matrices, e.g. agarose, dextran or cellulose, involves activation of the carrier with cyanogen halides and subsequent coupling of the peptide ligand's primary aliphatic or aromatic amines to the activated matrix.

Alternatively, Notch1 can be used to coat the wells of adsorption plates, expressed on host cells affixed to adsorption plates or used in cell sorting, or conjugated to biotin for capture with streptavidin-coated beads, or used in any other art-known method for panning phage display libraries.

The phage library samples are contacted with immobilized Notch1 under conditions suitable for binding of at least a portion of the phage particles with the adsorbent. Normally, the conditions, including pH, ionic strength, temperature and the like are selected to mimic physiological conditions. The phages bound to the solid phase are washed and then eluted by acid, e.g. as described in Barbas et al., Proc. Natl. Acad. Sci. USA, 88: 7978-7982 (1991), or by alkali, e.g. as described in Marks et al., J. Mol. Biol., 222: 581-597 (1991), or by Notch1 antigen competition, e.g. in a procedure similar to the antigen competition method of Clackson et al., Nature, 352: 624-628 (1991). Phages can be enriched 20-1,000-fold in a single round of selection. Moreover, the enriched phages can be grown in bacterial culture and subjected to further rounds of selection.

The efficiency of selection depends on many factors, including the kinetics of dissociation during washing, and whether multiple antibody fragments on a single phage can simultaneously engage with antigen. Antibodies with fast dissociation kinetics (and weak binding affinities) can be retained by use of short washes, multivalent phage display and high coating density of antigen in solid phase. The high density not only stabilizes the phage through multivalent interactions, but favors rebinding of phage that has dissociated. The selection of antibodies with slow dissociation kinetics (and good binding affinities) can be promoted by use of long washes and monovalent phage display as described in Bass et al., Proteins, 8: 309-314 (1990) and in WO 92/09690, and a low coating density of antigen as described in Marks et al., Biotechnol., 10: 779-783 (1992).

It is possible to select between phage antibodies of different affinities, even with affinities that differ slightly, for Notch1. However, random mutation of a selected antibody (e.g. as performed in some of the affinity maturation techniques described above) is likely to give rise to many mutants, most binding to antigen, and a few with higher affinity. With limiting Notch1, rare high affinity phage could be competed out. To retain all the higher affinity mutants, phages can be incubated with excess biotinylated Notch1, but with the biotinylated Notch1 at a concentration of lower molarity than the target molar affinity constant for Notch1. The high affinity-binding phages can then be captured by streptavidin-coated paramagnetic beads. Such “equilibrium capture” allows the antibodies to be selected according to their affinities of binding, with sensitivity that permits isolation of mutant clones with as little as two-fold higher affinity from a great excess of phages with lower affinity. Conditions used in washing phages bound to a solid phase can also be manipulated to discriminate on the basis of dissociation kinetics.

Anti-Notch1 clones may be activity selected. In one embodiment, the invention provides anti-Notch1 antibodies that block the binding between a Notch receptor (such as Notch1, Notch2, Notch3 and/or Notch4), preferably one of a Notch1 and/or Notch3 receptor and its binding partner. Fv clones corresponding to such anti-Notch1 antibodies can be selected by (1) isolating anti-Notch1 clones from a phage library as described above, and optionally amplifying the isolated population of phage clones by growing up the population in a suitable bacterial host; (2) selecting Notch1 and a second protein against which blocking and non-blocking activity, respectively, is desired; (3) adsorbing the anti-Notch1 phage clones to immobilized Notch1; (4) using an excess of the second protein to elute any undesired clones that recognize Notch1-binding determinants which overlap or are shared with the binding determinants of the second protein; and (5) eluting the clones which remain adsorbed following step (4). Optionally, clones with the desired blocking/non-blocking properties can be further enriched by repeating the selection procedures described herein one or more times.

DNA encoding, for example, phage display Fv clones of the invention is readily isolated and sequenced using conventional procedures (e.g. by using oligonucleotide primers designed to specifically amplify the heavy and light chain coding regions of interest from hybridoma or phage DNA template). Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of the desired monoclonal antibodies in the recombinant host cells. Review articles on recombinant expression in bacteria of antibody-encoding DNA include Skerra et al., Curr. Opinion in Immunol., 5: 256 (1993) and Pluckthun, Immunol. Revs, 130: 151 (1992).

DNA encoding the Fv clones of the invention can be combined with known DNA sequences encoding heavy chain and/or light chain constant regions (e.g. the appropriate DNA sequences can be obtained from Kabat et al., supra) to form clones encoding full or partial length heavy and/or light chains. It will be appreciated that constant regions of any isotype can be used for this purpose, including IgG, IgM, IgA, IgD, and IgE constant regions, and that such constant regions can be obtained from any human or animal species. A Fv clone derived from the variable domain DNA of one animal (such as human) species and then fused to constant region DNA of another animal species to form coding sequence(s) for “hybrid”, full length heavy chain and/or light chain is included in the definition of “chimeric” and “hybrid” antibody as used herein. In a preferred embodiment, a Fv clone derived from human variable DNA is fused to human constant region DNA to form coding sequence(s) for all human, full or partial length heavy and/or light chains.

### Antibody Fragments

In certain circumstances there are advantages of using antibody fragments, rather than whole antibodies. The smaller size of the fragments allows for rapid clearance, and may lead to improved access to solid tumors.

An antibody functional fragment refers to a portion of an antibody which retains some or all of its target-specific binding activity. Such functional fragments can include, for example, antibody functional fragments such as Fv, Fab, F(ab′), F(ab)₂, F(ab′)₂, single chain Fv (scFv), diabodies, triabodies, tetrabodies and minibody. Other functional fragments can include, for example, heavy (H) or light (L) chain polypeptides, variable heavy (V_{H}) and variable light (V_{L}) chain region polypeptides, complementarity determining region (CDR) polypeptides, single domain antibodies, and polypeptides that contain at least a portion of an immunoglobulin that is sufficient to retain target-specific binding activity. The present invention encompasses antibody fragments. In certain circumstances there are advantages of using antibody fragments, rather than whole antibodies. The smaller size of the fragments allows for rapid clearance, and may lead to improved access to solid tumors.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10: 163-167 (1992)). According to another approach, F(ab′)₂ fragments can be isolated directly from recombinant host cell culture. Fab and F(ab′)₂ fragment with increased in vivo half-life comprising a salvage receptor binding epitope residues are described in U.S. Pat. No. 5,869,046. Other techniques for the production of antibody fragments will be apparent to the skilled practitioner. In other embodiments, the antibody of choice is a single chain Fv fragment (scFv). See WO 93/16185; U.S. Pat. Nos. 5,571,894; and 5,587,458. Fv and sFv are the only species with intact combining sites that are devoid of constant regions; thus, they are suitable for reduced nonspecific binding during in vivo use. sFv fusion proteins may be constructed to yield fusion of an effector protein at either the amino or the carboxy terminus of an sFv. See Antibody Engineering, ed. Borrebaeck, supra. The antibody fragment may also be a “linear antibody”, e.g., as described in U.S. Pat. No. 5,641,870 for example. Such linear antibody fragments may be monospecific or bispecific.

With respect to antibodies and functional fragments thereof that exhibit beneficial binding characteristics to a target molecule, various forms, alterations and modifications are well known in the art. Target-specific monoclonal antibodies for use in a biopharmaceutical formulation of the invention can include any of such various monoclonal antibody forms, alterations and modifications. Examples of such various forms and terms as they are known in the art are set forth below.

### Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen (especially when synthetic peptides are used) to a protein that is immunogenic in the species to be immunized. For example, the antigen can be conjugated to keyhole limpet hemocyanin (KLH), serum albumin, bovine thyroglobutin, or soybean trypsin inhibitor, using a bifunctional or derivatizing agent, e.g., maleimidobenzoyl sulfosuccinimide ester (conjugation through cysteine residues). N-hydroxysuccinimide (through lysine residues), glutaraldehyde, succinic anhydride, SOCl₂, or R¹N═C═NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to 14 days later, the animals are bled and the serum is assayed for antibody titer. Animals are boosted until the titer plateaus. Conjugates also can be made in recombinant cell culture as protein fusions. Also, aggregating agents such as alum are suitably used to enhance the immune response.

### Monoclonal Antibodies

Monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium which medium preferably contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Preferred fusion partner myeloma cells are those that fuse efficiently, support stable high-level production of antibody by the selected antibody-producing cells, and are sensitive to a selective medium that selects against the unfused parental cells. Preferred myeloma cell lines are murine myeloma lines, such as those derived from MOPC-21 and MPC-11 mouse tumors available from the Salk Institute Cell Distribution Center, San Diego, Calif. USA, and SP-2 and derivatives e.g., X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The binding affinity of the monoclonal antibody can, for example, be determined by the Scatchard analysis described in Munson et al., Anal, Biochem., 107:220 (1980).

Once hybridoma cells that produce antibodies of the desired specificity, affinity, and/or activity are identified, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal e.g, by i.p. injection of the cells into mice.

The monoclonal antibodies secreted by the subclones are suitably separated from the culture medium, ascites fluid, or serum by conventional antibody purification procedures such as, for example, affinity chromatography (e. g., using protein A or protein G-Sepharose®) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Pluckthun, Immunol. Revs., 130:151-188 (1992).

In a further embodiment, monoclonal antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA that encodes the antibody may be modified, for example, by substituting human heavy chain and light chain constant domain (C_{H} and C_{L}) sequences for the homologous murine sequences (U.S. Pat. No. 4,816,567; and Morrison, et al., Proc. Natl Acad. Sci. USA, 81:6851 (1984)), or by fusing the immunoglobulin coding sequence with all or part of the coding sequence for a non-immunoglobulin polypeptide. The non-immunoglobulin polypeptide sequences can substitute for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

### Human Antibodies

Human anti-Notch1 antibodies of the invention can be constructed by combining Fv clone variable domain sequence(s) selected from human-derived phage display libraries with known human constant domain sequences(s) as described above. Alternatively, human monoclonal anti-Notch1 antibodies of the invention can be made by the hybridoma method. Human myeloma and mouse-human heteromyeloma cell lines for the production of human monoclonal antibodies have been described, for example, by Kozbor J. Immuunol., 133: 3001 (1984); Brodeur et al, Monoclonal Antibody Production Techniques and Applications, pp. 51-63 (Marcel Dekker, Inc., New York, 1987); and Boerner et al, J. Immunol., 147: 86 (1991).

It is now possible to produce transgenic animals (e.g. mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90: 2551 (1993); Jakobovits et al, Nature, 362: 255 (1993); Bruggermann et al, Year in Immunol., 7: 33 (1993); U.S. Pat. Nos. 5,545,806, 5,569,825, 5,591,669 (all of GenPharm); 5,545,807; and WO 97/17852.

Alternatively, phage display technology (McCafferty et al., Nature 348:552-553 (1990) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S. and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

Human antibodies may also be generated by in vitro activated B cells (see U.S. Pat. Nos. 5,567,610 and 5,229,275).

Gene shuffling can also be used to derive human antibodies from non-human, e.g. rodent, antibodies, where the human antibody has similar affinities and specificities to the starting non-human antibody. According to this method, which is also called “epitope imprinting”, either the heavy or light chain variable region of a non-human antibody fragment obtained by phage display techniques as described above is replaced with a repertoire of human V domain genes, creating a population of non-human chain/human chain scFv or Fab chimeras. Selection with antigen results in isolation of a non-human chain/human chain chimeric scFv or Fab wherein the human chain restores the antigen binding site destroyed upon removal of the corresponding non-human chain in the primary phage display clone, i.e. the epitope governs (imprints) the choice of the human chain partner. When the process is repeated in order to replace the remaining non-human chain, a human antibody is obtained (see PCT WO 93/06213 published Apr. 1, 1993). Unlike traditional humanization of non-human antibodies by CDR grafting, this technique provides completely human antibodies, which have no FR or CDR residues of non-human origin.

### Bispecific Antibodies

Bispecific antibodies are monoclonal antibodies that have binding specificities for at least two different antigens. In the present case, one of the binding specificities is for Notch1 and the other is for any other antigen. Exemplary bispecific antibodies may bind to two different epitopes of the Notch1 protein. Bispecific antibodies may also be used to localize cytotoxic agents to cells which express Notch1. These antibodies possess a Notch1-binding arm and an arm which binds the cytotoxic agent (e.g. saporin, anti-interferon-α, vinca alkaloid, ricin A chain, methotrexate or radioactive isotope hapten). Bispecific antibodies can be prepared as full length antibodies or antibody fragments (e.g. F(ab′)₂ bispecific antibodies).

Methods for making bispecific antibodies are known in the art. Traditionally, the recombinant production of bispecific antibodies is based on the co-expression of two immunoglobulin heavy chain-light chain pairs, where the two heavy chains have different specificities (Milstein and Cuello, Nature, 305: 537 (1983)). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed in WO 93/08829 published May 13, 1993, and in Traunecker et al., EMBO J., 10: 3655 (1991).

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1), containing the site necessary for light chain binding, present in at least one of the fusions. DNAs encoding the immunoglobulin heavy chain fusions and, if desired, the immunoglobulin light chain, are inserted into separate expression vectors and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In one embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach, the interface between a pair of antibody molecules can be engineered to maximize the percentage of heterodimers which are recovered from recombinant cell culture. The preferred interface comprises at least a part of the C_{H3} domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Various techniques for making and isolating bispecific antibody fragments directly from recombinant cell culture have also been described. For example, bispecific antibodies have been produced using leucine zippers. See Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. Another strategy for making bispecific antibody fragments by the use of single-chain Fv (scFv) dimers has also been reported. See Gruber et al., J. Immunol., 152:5368 (1994).

### Heteroconjugate

Bispecific antibodies include cross-linked or “heteroconjugate” antibodies. As such, heteroconjugate antibodies are also within the scope of the present invention. Heteroconjugate antibodies are composed of two covalently joined antibodies. Such antibodies have, for example, been proposed to target immune system cells to unwanted cells, e.g., U.S. Pat. No. 4,676,980, and for treatment of HIV infection, e.g., WO 91/00360; WO 92/200373; EP 03089. Heteroconjugate antibodies may be made using any convenient cross-linking methods. It is contemplated that the antibodies may be prepared in vitro using known methods in synthetic protein chemistry, including those involving cross-linking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

### Diabody

The “diabody” technology described by Hollinger et al., Proc. Natl. Acad. Sci. USA, 90:6444-6448 (1993) has provided an alternative mechanism for making bispecific antibody fragments. A diabody is a bivalent dimer formed by the non-covalent association of two scFvs, yielding two Fv binding sites. Briefly, a diabody refers to an engineered antibody construct prepared by isolating the binding domains (both heavy and light chain) of a binding antibody, and supplying a linking moiety which joins or operably links the heavy and light chains on the same polypeptide chain thereby preserving the binding function (see, Holliger et al. (1993) Proc. Natl. Acad. Sci. USA 90:6444; Poljak (1994) Structure 2:1121-1123). This forms, in essence, a radically abbreviated antibody, having only the variable domain necessary for binding the antigen. By using a linker that is too short to allow pairing between the two domains on the same chain, the domains are forced to pair with the complementary domains of another chain and create two antigen-binding sites. These dimeric antibody fragments, or diabodies, are bivalent and bispecific. Thus, diabodies are dimers of two scFv molecules that cannot fold properly into one scFv molecule. Diabodies are built like scFv molecules, but usually have a short (less than 10, preferably 1-5 amino acids) peptide linker connecting both V-domains, whereby both domains can not interact intramolecular, and are forced to interact intermolecular (Holliger et al., 1993) (U.S. Pat. No. 5,837,242). A diabody thus may consist of a VH-VL chain that interacts with a similar VH-VL chain to form a dimer of the formula VH-VL:VH-VL. The diabody chain dimers bind the antigen specified by VH and VL bivalent. Winter described the construction of bispecific diabodies by coupling the VH domain of a chosen antibody A to the VL domain of a chosen antibody B, using a peptide linker sufficiently short to inhibit the interaction of VH(A) with VL(B). Also the reverse molecule VH(B)-VL(A) is made the same way (Holliger, Griffiths, Hoogenboom, Malmqvist, Marks, McGuinness, Pope, Prospero and Winter: “Multivalent and multispecific binding proteins, their manufacture and use”, U.S. Pat. No. 5,837,242, 1998). The skilled artisan will appreciate that any method to generate diabodies can be used. Suitable methods are described by Holliger, et al. (1993) supra, Poljak (1994) supra, Zhu, et al. (1996) Biotechnology 14:192-196, and U.S. Pat. No. 6,492,123, incorporated herein by reference.

### Fab′-SH

Recent progress has facilitated the direct recovery of Fab′-SH fragments from E. coli, which can be chemically coupled to form bispecific antibodies. Shalaby et al., J. Exp. Med., 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the HER2 receptor and normal human T cells, as well as trigger the lytic activity of human cytotoxic lymphocytes against human breast tumor targets.

### Trispecific

Antibodies with more than two valencies are contemplated. For example, trispecific antibodies can be prepared. Tutt et al. J. Immunol. 147: 60 (1991).

### Tetravalent

Tetravalent bispecific antibodies can be created by chemical cross-linking of two monoclonal antibodies (Bs(IgG)2) (Karpovsky et al., 1984) (U.S. Pat. No. 4,676,980). Problems related to their rapid clearance in vivo via the kidney due to their small size may be circumvented by, for example, increasing their molecular weight size thereby increasing their serum permanence and product efficacy. (Wu, A. M., Chen, W., Raubitschek, A., Williams, L. E., Neumaier, M., Fischer, R., Hu, S. Z., Odom-Maryon, T., Wong, J. Y. and Shively, J. E.: Tumor localization of anti-CEA single-chain Fvs: improved targeting by non-covalent dimers. Immunotechnology 2 (1996) 21-36).

### Peptibodies

Peptibodies, which consist of an immunoglobulin constant region domain (Fc) linked to two binding peptides through either the carboxyl- or amino termini of the Fc domain, also are included herein as an antibody functional fragment. Such antibody binding fragments can be found described in, for example, Harlow and Lane, supra; Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, Second Ed., Wiley-Liss, Inc., New York, N.Y. (1990).

### Multivalent Antibodies

A multivalent antibody may be internalized (and/or catabolized) faster than a bivalent antibody by a cell expressing an antigen to which the antibodies bind. The antibodies of the present invention can be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fe region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1)n-VD2-(X2)n-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

### Antibody Variants

In some embodiments, amino acid sequence modification(s) of the antibodies described herein are contemplated. For example, it may be desirable to improve the binding affinity and/or other biological properties of the antibody. Amino acid sequence variants of the antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics. The amino acid alterations may be introduced in the subject antibody amino acid sequence at the time that sequence is made.

A useful method for identification of certain residues or regions of the antibody that are preferred locations for mutagenesis is called “alanine scanning mutagenesis” as described by Cunningham and Wells (1989) Science, 244:1081-1085. Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed immunoglobulins are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion of the N- or C-terminus of the antibody to an enzyme (e.g. for ADEPT) or a polypeptide which increases the serum half-life of the antibody.

Glycosylation of polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites). See section marked “Effector Function Engineering”, infra.

Where the antibody comprises an Fc region, the carbohydrate attached thereto may be altered. For example, antibodies with a mature carbohydrate structure that lacks fucose attached to an Fc region of the antibody are described in US Pat Appl No US 2003/0157108 (Presta, L.). See also US 2004/0093621 (Kyowa Hakko Kogyo Co., Ltd). Antibodies with a bisecting N-acetylglucosamine (GlcNAc) in the carbohydrate attached to an Fc region of the antibody are referenced in WO 2003/011878, Jean-Mairet et al. and U.S. Pat. No. 6,602,684, Umana et al. Antibodies with at least one galactose residue in the oligosaccharide attached to an Fc region of the antibody are reported in WO 1997/30087, Patel et al. See, also, WO 1998/58964 (Raju, S.) and WO 1999/22764 (Raju, S.) concerning antibodies with altered carbohydrate attached to the Fc region thereof. See also US 2005/0123546 (Umana et al.) on antigen-binding molecules with modified glycosylation.

At least one glycosylation variant herein comprises an Fc region, wherein a carbohydrate structure attached to the Fc region lacks fucose. Such variants have improved ADCC function. Optionally, the Fc region further comprises one or more amino acid substitutions therein which further improve ADCC, for example, substitutions at positions 298, 333, and/or 334 of the Fc region (Eu numbering of residues). Examples of publications related to “defucosylated” or “fucose-deficient” antibodies include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; Okazaki et al. J. Mol. Biol. 336:1239-1249 (2004); Yamane-Ohnuki et al. Biotech. Bioeng. 87: 614 (2004). Examples of cell lines producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (Yamane-Ohnuki et al. Biotech. Bioeng. 87: 614 (2004)). For further details, see “Effector Function Engineering” infra.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative substitutions are shown in Table 1 under the heading of “preferred substitutions”. If such substitutions result in a change in biological activity, then more substantial changes, denominated “exemplary substitutions” in Table 1, or as further described below in reference to amino acid classes, may be introduced and the products screened.

Exemplary Preferred Residue Substitutions Ala (A) Val; Leu; Ile Val Arg (R) Lys; Gln; Asn Lys Asn (N) Gln; His; Asp, Lys; Arg Gln Asp (D) Glu; Asn Glu Cys (C) Ser; Ala Ser Gln (Q) Asn; Glu Asn Glu (E) Asp; Gln Asp Gly (G) Ala Ala His (H) Asn; Gln; Lys; Arg Arg Ile (I) Leu; Val; Met; Ala; Leu Phe; Norleucine Leu (L) Norleucine; Ile; Val; Ile Met; Ala; Phe Lys (K) Arg; Gln; Asn Arg Met (M) Leu; Phe; Ile Leu Phe (F) Trp; Leu; Val; Ile; Ala; Tyr Tyr Pro (P) Ala Ala Ser (S) Thr Thr Thr (T) Val; Ser Ser Trp (W) Tyr; Phe Tyr Tyr (Y) Trp; Phe; Thr; Ser Phe Val (V) Ile; Leu; Met; Phe; Leu Ala; Norleucine

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro; and

(6) aromatic: Trp, Tyr, Phe.

Non-conservative substitutions will entail exchanging a member of one of these classes for another class.

One type of substitutional variant involves substituting one or more hypervariable region residues of a parent antibody (e.g. human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g. 6-7 sites) are mutated to generate all possible amino acid substitutions at each site. The antibodies thus generated are displayed from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

### Effector Function Engineering

(A) Anti-Notch1 Antibodies with Variant Fc Regions

Most immune functions of antibodies depend on their ability to act as flexible adaptor molecules, linking pathogen with appropriate elimination mechanisms. This ‘bridging’ role entails two types of recognition, each involving contributions from particular antibody domains. The first involves highly specific recognition of the antigen target, and is mediated through the amino-terminal variable domains of the two Fab regions of the antibody. The second involves interaction of the constant domains of the Fc region of the molecule with various effector molecules, including complement and, perhaps most importantly, Fc receptors (FcRs) present on phagocytes and other immune cells. The dual recognition of target and FcR by immunoglobulin molecules has a key role in eliciting effector mechanisms to rid the body of bacteria, viruses, and parasites.

Briefly, therapeutic antibodies can exert potent biological functions through two major non-exclusive mechanisms: (i) they can block interactions between receptors and their ligands due to the exquisite epitope specificity of their variable domains (“neutralizing/antagonist antibodies”) or trigger potent biological responses such as apoptosis or cell proliferation once they are bound to surface molecules (“agonist antibodies”); (ii) induce effector functions against pathogens and tumor cells following their interactions with the complement component C1q and/or with receptors for Fc region (FcγR). See Cragg et al., Curr Opin Immunol 11:541-547 (1999); Glennie et al., Immunol Today 21:403-410 (2000).

The effector functions of immunoglobulins e.g., IgG, which is the most common immunoglobulin, are mediated by the antibody Fc region through two major mechanisms: (1) binding to the cell surface Fc receptors (FcγRs) can lead to ingestion of pathogens by phagocytosis or lysis by killer cells via the antibody-dependent cellular cytotoxicity (ADCC) pathway, or (2) binding to the C1q part of the first complement component C1 initiates the complement-dependent cytotoxicity (CDC) pathway, resulting in the lysis of pathogens. Reviewed in Daeron, Annu Rev. Immunol. 15:203-234 (1997); Ward and Ghetie, Therapeutic Immunol. 2:77-94 (1995); Ravetch and Kinet, Annu Rev. Immunol. 9:457-492 (1991). Uananue and Benacerraf, Textbook of Immunology, 2nd Edition, Williams & Wilkins, p. 218 (1984)). There are three known FcγRs, designated FcγRI (CD64), FcγRII (CD32), and FcγRIII (CD16). Anti-tumor efficacy can be due to a combination of these mechanisms, and their relative importance in clinical therapy appears to be cancer dependent. Notwithstanding this arsenal of anti-tumor weapons, the potency of antibodies as anti-cancer agents is unsatisfactory, particularly given their high cost. Currently for anti-cancer therapy, any small improvement in mortality rate defines success.

Thus, it may be desirable to introduce one or more amino acid modifications in an Fc region of the immunoglobulin polypeptides of the invention, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions including that of a hinge cysteine.

ADCC involves the recognition of the antibody by immune cells that engage the antibody-marked cells and either through their direct action, or through the recruitment of other cell types, leads to the tagged-cell's death. CDC is a process where a cascade of different complement proteins become activated, usually when several IgGs are in close proximity to each other, either with one direct outcome being cell lysis, or one indirect outcome being attracting other immune cells to this location for effector cell function.

A promising means for enhancing the anti-tumor potency of antibodies is via enhancement of their ability to mediate cytotoxic effector functions such as ADCC, ADCP, and CDC. The importance of ADCC as a cytotoxic mechanism of anti-tumor mAbs has been demonstrated in animal studies. Ravetch et al., Annu Rev. Immunol. 16:421-432 (1998) showed that the tumoricidal effect of a humanized anti-HER2/neu mAb (epithelial growth factor receptor 2; Trastuzumab) was significantly reduced in FcγR knockout nude mice as compared to wild-type nude mice. Similarly, the tumor regression activity of a chimeric anti-CD20 mAb (Rituximab) was significantly reduced in FcγR deficient mice as compared to wild-type mice. Ravetch, supra; Clynes et al., Inhibitory Fc receptors modulate in vivo cytoxicity against tumor targets. Nat. Med. 6:443-446 (2000). Further support for an important role for ADCC was provided by a study of Cartron et al., who found that in patients with a polymorphism in FcγRIIIa leading to increased binding of IgG1, therapy with an anti-CD20 mAb produced a 90% response rate (patients with complete remission or partial response) at 12 months, compared to a 51% response rate in individuals not expressing this polymorphism of FcγRIIIa. Cartron et al., Therapeutic activity of humanized anti-CD20 monoclonal antibody and polymorphism in IgG Fc receptor FcγRIIIa gene, Blood 99:754-758 (2002). Others have shown that this FcγRIIIa polymorphism and also a polymorphism in FcγRIIa are associated with the response rate to therapeutic mAbs. W. K. Weng and R. Levy, Two immunoglobulin G fragment C receptor polymorphisms independently predict response to rituximab in patients with follicular lymphoma. J. Clin. Oncol. 21:3940-3947 (2003). The importance of FcγR-mediated effector functions for the anti-cancer activity of antibodies has also been demonstrated in mice (Clynes et al., 1998, Proc Natl Acad Sci USA 95:652-656; Clynes et al., 2000, Nat Med 6:443-446), and the affinity of interaction between Fc and certain FcγRs correlates with targeted cytotoxicity in cell-based assays (Shields et al., 2001, J Biol Chem 276:6591-6604; Presta et al., 2002, Biochem Soc Trans 30:487-490; Shields et al., 2002, J Biol Chem 277:26733-26740). Additionally, a correlation has been observed between clinical efficacy in humans and their allotype of high (V158) or low (F158) affinity polymorphic forms of FcγRIIIa (Cartron et al., 2002, Blood 99:754-758). Together these data suggest that an antibody that is optimized for binding to certain FcγRs may better mediate effector functions and thereby destroy cancer cells more effectively in patients. The balance between activating and inhibiting receptors is an important consideration, and optimal effector function may result from an antibody that has enhanced affinity for activation receptors, for example FcγRI, FcγRIIa/c, and FcγRIIIa, yet reduced affinity for the inhibitory receptor FcγRIIb. Furthermore, because FcγRs can mediate antigen uptake and processing by antigen presenting cells, enhanced FcγR affinity may also improve the capacity of antibody therapeutics to elicit an adaptive immune response. Fc variants have been successfully engineered with selectively enhanced binding to FcγRs, and furthermore these Fc variants provide enhanced potency and efficacy in cell-based effector function assays. See for example U.S. Ser. No. 10/672,280, U.S. Ser. No. 10/822,231, entitled “Optimized Fc Variants and Methods for their Generation”, U.S. Ser. No. 60/627,774, entitled “Optimized Fc Variants”, and U.S. Ser. No. 60/642,477, entitled “Improved Fc Variants”, and references cited therein. U.S. Pat. No. 7,276,585 Xencor, Inc. (Monrovia, Calif.) See also U.S. Pat. No. 6,821,505.

Most mAbs that mediate antibody-dependent cellular cytotoxicity (ADCC) also activate the complement system 1. A. Gorter and S. Meri, “Immune evasion of tumor cells using membrane-bound complement regulatory proteins.” Immunol. Today, pp. 576-582 (1999).

Complement initiates three mechanisms that can be used against mAb-coated tumor cells. The first is direct complement killing of tumor cells by the membrane attack complex (MAC), a process usually called ‘complement-dependent cytotoxicity’ (CDC). The second mechanism is complement receptor-dependent enhancement of ADCC. In this case, CR3 binds to iC3b, thus enhancing FcγR-mediated effector cell binding. A third mechanism used for killing microorganisms, CR3-dependent cellular cytotoxicity (CR3-DCC), is usually not activated with tumors.

Based upon the results of chemical modifications and crystallographic studies, Burton et al. (Nature, 288:338-344 (1980)) proposed that the binding site for the complement subcomponent C1q on IgG involves the last two (C-terminal) β-strands of the CH2 domain. Burton later suggested (Molec. Immunol., 22(3):161-206 (1985)) that the region comprising amino acid residues 318 to 337 might be involved in complement fixation.

Duncan and Winter (Nature 332:738-40 (1988)), using site directed mutagenesis, reported that Glu318, Lys320 and Lys322 form the binding site to C1q. The data of Duncan and Winter were generated by testing the binding of a mouse IgG2b isotype to guinea pig C1q. The role of Glu318, Lys320 and Lys322 residues in the binding of C1q was confirmed by the ability of a short synthetic peptide containing these residues to inhibit complement mediated lysis. Similar results are disclosed in U.S. Pat. No. 5,648,260 issued on Jul. 15, 1997, and U.S. Pat. No. 5,624,821 issued on Apr. 29, 1997.

The residue Pro331 has been implicated in C1q binding by analysis of the ability of human IgG subclasses to carry out complement mediated cell lysis. Mutation of Ser331 to Pro331 in IgG4 conferred the ability to activate complement. (Tao et al., J. Exp. Med., 178:661-667 (1993); Brekke et al., Eur. J. Immunol., 24:2542-47 (1994)).

From the comparison of the data of the Winter group, and the Tao et al. and Brekke et al. papers, Ward and Ghetie concluded in their review article that there are at least two different regions involved in the binding of C1q: one on the β-strand of the CH2 domain bearing the Glu318, Lys320 and Lys322 residues, and the other on a turn located in close proximity to the same β-strand, and containing a key amino acid residue at position 331.

Other reports suggest that human IgG1 residues Leu235, and Gly237, located in the lower hinge region, play a critical role in complement fixation and activation. Xu et al., J. Immunol. 150:152A (Abstract) (1993). WO94/29351 published Dec. 22, 1994 reports that amino acid residues necessary for C1q and FcR binding of human IgG1 are located in the N-terminal region of the CH2 domain, i.e. residues 231 to 238.

It has further been proposed that the ability of IgG to bind C1q and activate the complement cascade also depends on the presence, absence, or modification of the carbohydrate moiety positioned between the two CH2 domains (which is normally anchored at Asn297). Ward and Ghetie, Therapeutic Immunology 2:77-94 (1995) at page 81.

The binding site on human and murine antibodies for FcγR have been previously mapped to the so-called “lower hinge region” consisting of residues 233-239 (EU index numbering as in Kabat et al., Sequences of Proteins of Immunological Interest, 5th Ed. Public Health Service, National Institutes of Health, Bethesda, Md. (1991)). Woof et al. Molec. Immunol. 23:319-330 (1986); Duncan et al. Nature 332:563 (1988); Canfield and Morrison, J. Exp. Med. 173:1483-1491 (1991); Chappel et al., Proc. Natl. Acad. Sci USA 88:9036-9040 (1991). Of residues 233-239, P238 and S239 have been cited as possibly being involved in binding, but these two residues have never been evaluated by substitution or deletion.

Other previously cited areas possibly involved in binding to FcγR: G316-K338 (human IgG) for human FcγRI (by sequence comparison only; no substitution mutants were evaluated) (Woof et al. Molec. Immunol 23:319-330 (1986)); K274-R301 (human IgG1) for human FcγRIII (based on peptides) (Sarmay et al. Molec. Immunol. 21:43-51 (1984)); Y407-R416 (human IgG) for human FcγRIII (based on peptides) (Gergely et al. Biochem. Soc. Trans. 12:739-743 (1984)).

U.S. Pat. No. 6,165,745 discloses a method of producing an antibody with a decreased biological half-life by introducing a mutation into the DNA segment encoding the antibody. The mutation includes an amino acid substitution at position 253, 310, 311, 433, or 434 of the Fc-hinge domain. The full disclosure of U.S. Pat. No. 6,165,745, as well as the full disclosure of all other U.S. patent references cited herein, are hereby incorporated by reference.

U.S. Patent Application No. 20020098193 A1 and PCT Publication No. WO 97/34621 disclose mutant IgG molecules having increased serum half-lives relative to IgG wherein the mutant IgG molecule has at least one amino acid substitution in the Fc-hinge region. However, no experimental support is provided for mutations at positions 250, 314, or 428.

U.S. Pat. No. 6,277,375 B1 discloses a composition comprising a mutant IgG molecule having an increased serum half-life relative to the wild-type IgG, wherein the mutant IgG molecule comprises the amino acid substitutions: threonine to leucine at position 252, threonine to serine at position 254, or threonine to phenylalanine at position 256. A mutant IgG with an amino acid substitution at position 433, 435, or 436 is also disclosed.

U.S. Pat. No. 6,528,624 discloses a variant of an antibody comprising a human IgG Fc region, which variant comprises an amino acid substitution at one or more of amino acid positions 270, 322, 326, 327, 329, 331, 333, and 334 of the human IgG Fc region.

In accordance with the above description and the teachings of the art, it is contemplated that in some embodiments, an antibody used in methods of the invention may comprise one or more alterations as compared to the wild type counterpart antibody, e.g. in the Fc region. These antibodies would nonetheless retain substantially the same characteristics required for therapeutic utility as compared to their wild type counterpart. For example, it is thought that certain alterations can be made in the Fc region that would result in altered (i.e., either improved or diminished) C1q binding and/or Complement Dependent Cytotoxicity (CDC), e.g., as described in WO99/51642. See also Duncan & Winter Nature 322:738-40 (1988); U.S. Pat. No. 5,648,260; U.S. Pat. No. 5,624,821; and WO94/29351 concerning other examples of Fc region variants. WO00/42072 (Presta) and WO 2004/056312 (Lowman) describe antibody variants with improved or diminished binding to FcRs. The content of these patent publications are specifically incorporated herein by reference. See, also, Shields et al. J. Biol. Chem. 9(2): 6591-6604 (2001). Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934 μl (Hinton et al.). These antibodies comprise an Fc region with one or more substitutions therein which improve binding of the Fc region to FcRn. Polypeptide variants with altered Fc region amino acid sequences and increased or decreased C1q binding capability are described in U.S. Pat. No. 6,194,551B1, WO99/51642. The contents of those patent publications are specifically incorporated herein by reference. See, also, Idusogie et al. J. Immunol. 164:4178-4184 (2000).

Functional Assays of Molecules with Variant Fc Regions

The ability of any particular antibody e.g., any one or more of the anti-antibodies disclosed herein, to mediate lysis of the target cell by complement activation and/or ADCC can be assayed. Functional assays for identifying potent Fc variants of any one or more of the anti-Notch1 antibodies of the invention are well known to one skilled in the art. See, for example, U.S. Patent Application Publications 2005/0037000 and 2005/0064514, and International Patent Application Publication WO 04/063351 (each of which is hereby incorporated by reference in its entirety); that describe yeast display technology for characterizing an antibody with a variant Fc region. Likewise, R-Fc binding assays are disclosed in U.S. Patent Application Publications 2005/0037000 and 2005/0064514, and International Patent Application Publication WO 04/063351 (each of which is hereby incorporated by reference in its entirety).

Examples of effector cell functions that can be assayed in accordance with the invention, include but are not limited to, antibody-dependent cell mediated cytotoxicity, phagocytosis, opsonization, opsonophagocytosis, C1q binding, and complement dependent cell mediated cytotoxicity. Any cell-based or cell free assay known to those skilled in the art for determining effector cell function activity can be used (For effector cell assays, see Perussia et al., 2000, Methods Mol. Biol. 121: 179-92; Baggiolini et al., 1998 Experientia, 44(10): 841-8; Lehmann et al., 2000 J. Immunol. Methods, 243(1-2): 229-42; Brown E J. 1994, Methods Cell Biol., 45: 147-64; Munn et al., 1990 J. Exp. Med., 172: 231-237, Abdul-Majid et al., 2002 Scand. J. Immunol. 55: 70-81; Ding et al., 1998, Immunity 8:403-411, each of which is incorporated by reference herein in its entirety).

Generally, the cells of interest are grown and labeled in vitro; the target antibody is added to the cell culture in combination with either serum complement or immune cells which may be activated by the antigen-antibody complexes. Cytolysis of the target cells is detected by the release of label from the lysed cells. In fact, antibodies can be screened using the patient's own serum as a source of complement and/or immune cells. The antibody that is capable of activating complement or mediating ADCC in the in vitro test can then be used therapeutically in that particular patient.

Preferably, the effector cells used in the ADCC assays of the invention are peripheral blood mononuclear cells (PBMC) that are preferably purified from normal human blood, using standard methods known to one skilled in the art, e.g. using Ficoll-Paque density gradient centrifugation.

An exemplary assay for determining ADCC activity of such anti-Notch1 antibodies with variant Fc regions is based on a 51Cr release assay comprising of: labeling target cells with [51Cr]Na2CrO4 (this cell-membrane permeable molecule is commonly used for labeling since it binds cytoplasmic proteins and although spontaneously released from the cells with slow kinetics, it is released massively following target cell necrosis); osponizing the target cells with the anti-antibodies with variant Fc region(s) of the invention; combining the opsonized radiolabeled target cells with effector cells in a microtitre plate at an appropriate ratio of target cells to effector cells; incubating the mixture of cells for 16-18 hours at 37° C.; collecting supernatants; and analyzing radioactivity. The cytotoxicity of the anti-antibodies with variant Fc regions can then be determined using known formulae, for example using the following formula: % lysis=(experimental cpm−target leak cpm)/(detergent lysis cpm−target leak cpm).times.100%. Alternatively, % lysis=(ADCC−AICC)/(maximum release−spontaneous release). Specific lysis can be calculated using the formula: specific lysis=% lysis with the anti-antibodies with variant Fc region(s) of the invention-% lysis in the absence of the anti-antibodies with variant Fc region(s) of the invention. A graph can be generated by varying either the target:effector cell ratio or antibody concentration. Perussia et al., 2000, Methods Mol. Biol. 121: 179-92.

The affinities and binding properties of anti-antibodies with variant Fc regions for an FcγR may initially be determined using in vitro assays (biochemical or immunological based assays) known in the art for determining Fc-FcγR interactions, i.e., specific binding of an Fc region to an FcγR including but not limited to ELISA assay, surface plasmon resonance assay, immunoprecipitation assays. Preferably, the binding properties of the anti-antibodies with variant Fc regions in accordance with the invention may also be characterized by in vitro functional assays for determining one or more FcγR mediator effector cell functions. In some embodiments, the anti-Notch Fc variants of the invention have similar binding properties in in vivo models as those in in vitro based assays. However, the present invention does not exclude molecules of the invention that do not exhibit the desired phenotype in in vitro based assays but do exhibit the desired phenotype in vivo.

Methods for generating anti-antibodies with variant Fc regions are known. DNA encoding an amino acid sequence variant of any one or more of the herein disclosed starting anti-Notch antibodies may be prepared by a variety of methods known in the art. These methods include, but are not limited to, preparation by site-directed (or oligonucleotide-mediated) mutagenesis, PCR mutagenesis, and cassette mutagenesis of an earlier prepared DNA encoding the antibody. In an alternative embodiment of the invention, however, a nucleic acid encoding an Fc region of a parent antibody is available and this nucleic acid sequence is altered to generate a variant nucleic acid sequence encoding the Fc region variant.

Site-directed mutagenesis is a preferred method for preparing substitution variants. This technique is well known in the art (see, e.g., Carter et al. Nucleic Acids Res. 13:4431-4443 (1985) and Kunkel et al, Proc. Natl. Acad. Sci. USA 82:488 (1985)). Briefly, in carrying out site-directed mutagenesis of DNA, the starting DNA is altered by first hybridizing an oligonucleotide encoding the desired mutation to a single strand of such starting DNA. After hybridization, a DNA polymerase is used to synthesize an entire second strand, using the hybridized oligonucleotide as a primer, and using the single strand of the starting DNA as a template. Thus, the oligonucleotide encoding the desired mutation is incorporated in the resulting double-stranded DNA.

PCR mutagenesis is also suitable for making amino acid sequence variants of the starting polypeptide. See Higuchi, in PCR Protocols, pp. 177-183 (Academic Press, 1990); and Vallette et al., Nuc. Acids Res. 17:723-733 (1989). Briefly, when small amounts of template DNA are used as starting material in a PCR, primers that differ slightly in sequence from the corresponding region in a template DNA can be used to generate relatively large quantities of a specific DNA fragment that differs from the template sequence only at the positions where the primers differ from the template.

Another method for preparing variants, cassette mutagenesis, is based on the technique described by Wells et al., Gene 34:315-323 (1985). The starting material is the plasmid (or other vector) comprising the starting polypeptide DNA to be mutated. The codon(s) in the starting DNA to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in the starting polypeptide DNA. The plasmid DNA is cut at these sites to linearize it. A double-stranded oligonucleotide encoding the sequence of the DNA between the restriction sites but containing the desired mutation(s) is synthesized using standard procedures, wherein the two strands of the oligonucleotide are synthesized separately and then hybridized together using standard techniques. This double-stranded oligonucleotide is referred to as the cassette. This cassette is designed to have 5′ and 3′ ends that are compatible with the ends of the linearized plasmid, such that it can be directly ligated to the plasmid. This plasmid now contains the mutated DNA sequence.

Alternatively, or additionally, the desired amino acid sequence encoding an anti-Fc variant can be determined, and a nucleic acid sequence encoding such amino acid sequence variant can be generated synthetically.

In certain embodiments, the modification entails one or more amino acid substitutions. The substitution may, for example, be a “conservative substitution”.

In some embodiments, the molecules of the invention with altered affinities for activating and/or inhibitory receptors having variant Fc regions, have one or more amino acid modifications.

The Fc regions of any one or more of the herein disclosed anti-antibodies may be optimized for a variety of properties. Properties that may be optimized include but are not limited to enhanced or reduced affinity for an FcγR. In one embodiment, the Fc variants are optimized to possess enhanced affinity for a human activating FcγR, preferably FcγRI, FcγRIIa, FcγRIIc, FcγRIIIa, and FcγRIIIb, most preferably FcγRIIIa. In an alternative embodiment, the Fc region is optimized to possess reduced affinity for the human inhibitory receptor FcγRIIb. These embodiments are anticipated to provide anti-antibodies with enhanced therapeutic properties in humans, for example enhanced effector function and greater anti-cancer potency.

In an alternate embodiment, the Fc variants of the present invention are optimized to have reduced or ablated affinity for a human FcγRI (should this be FcγR), including but not limited to FcγRI, FcγRIIa, FcγRIIb, FcγRIIc, FcγRIIIa, and IFcγRIIb (should this be FcγRIIIb. These embodiments are anticipated to provide anti-Notch1 antibodies with enhanced therapeutic properties in humans, for example reduced effector function and reduced toxicity. As is known in the art, cancer cells can be grafted or injected into mice to mimic a human cancer, a process referred to as xenografting. Testing of any one or more of the anti-Notch1 antibodies that comprise Fc variants that are optimized for one or more mouse FcγR, may provide valuable information with regard to the efficacy of the antibody, its mechanism of action, and the like.

The Fc variants of the present invention may also be optimized for enhanced functionality and/or solution properties in aglycosylated form. In one embodiment, the aglycosylated Fc variants of the present invention bind an Fc ligand with greater affinity than the aglycosylated form of the parent Fc polypeptide. Exemplary Fc ligands include but are not limited to FcγRs, C1q, FcRn, and proteins A and G, and may be from any source, preferably human. In an alternative embodiment, the Fc variants of the invention are optimized to be more stable and/or more soluble than the aglycosylated form of the parent Fc polypeptide.

Certain aspects of this invention thus involve antibodies which are (a) directed against a particular antigen and (b) belong to a subclass or isotype that is capable of mediating the lysis of cells to which the antibody molecule binds. More specifically, these antibodies should belong to a subclass or isotype that, upon complexing with cell surface proteins, activates serum complement and/or mediates antibody dependent cellular cytotoxicity (ADCC) by activating effector cells such as natural killer cells or macrophages. Towards this end, it may be desirable to modify the antibody of the invention with respect to effector function, so as to enhance the effectiveness of the antibody in treating cancer, for example. For example, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, any one or more of the anti-antibodies of the invention can be engineered with dual Fc regions and may thereby have enhanced complement lysis and ADCC capabilities. See Stevenson et al. Anti-Cancer Drug Design 3:219-230 (1989).

In yet another embodiment, the anti-antibodies with variant Fc region(s) of the invention are characterized for antibody dependent cellular cytotoxicity (ADCC) see, e.g., Ding et al, Immunity, 1998, 8:403-11; which is incorporated herein by reference in its entirety.

In another example, one or more amino acids in the Fc region can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

A broad aspect of the invention thus relates to immunoglobulins (e.g., anti-antibodies disclosed herein), comprising a variant Fc region, having one or more amino acid modifications (e.g., substitutions, but also including insertions or deletions) in one or more regions, which modifications alter, e.g., increase or decrease, the affinity of the variant Fc region for an FcγR. As binding to FcγRIIb decreases ADCC, it is important to increase binding to FcγRIIIA and decrease binding to FcγRIIB. Thus, in certain embodiments, said one or more amino acid modification increases the affinity of the variant Fc region for FcγRIIIA and/or FcγRIIA.

In certain embodiments, the herein described anti-antibodies with a variant Fc region further specifically bind FcγRIIB (via the Fc region) with a lower affinity than a comparable antibody molecule (i.e., having the same amino acid sequence as the antibody with a variant Fc region except for the one or more amino acid modifications in the Fc region) comprising the wild-type Fc region binds FcγRIIB.

In some embodiments, the invention encompasses molecules comprising a variant Fc region, wherein said variant Fc region comprises at least one amino acid modification relative to a wild type Fc region, which variant Fc region does not bind any FcγR or binds with a reduced affinity, relative to a comparable molecule comprising the wild-type Fc region, as determined by standard assays (e.g., in vitro assays) known to one skilled in the art. In a specific embodiment, the invention encompasses molecules comprising a variant Fc region, wherein said variant Fc region comprises at least one amino acid modification relative to a wild type Fc region, which variant Fc region only binds one FcγR, wherein the FcγR is FcγRIIIA. In another specific embodiment, the invention encompasses molecules comprising a variant Fc region, wherein said variant Fc region comprises at least one amino acid modification relative to a wild type Fc region, which variant Fc region only binds one FcγR wherein the FcγR is FcγRIIA. In yet another embodiment, the invention encompasses an anti-antibody molecule comprising a variant Fc region, wherein the variant Fc region comprises at least one amino acid modification relative to a wild type Fc region, which variant Fc region only binds one FcγR wherein the FcγR is FcγRIIB.

In yet another embodiment, provided herein is at least one or more anti-antibodies which comprises an antigen binding region and a variant Fe region, wherein the variant Fc region: (A) differs from a wild-type Fc region by comprising at least one amino acid modification according to the EU index as in Kabat, relative to the wild-type Fc region (unmodified), e.g., any one or more corresponding anti-antibodies disclosed herein that include the wild type Fc polypeptide; and (B) binds an FcγR with an increased affinity relative to a said wild-type Fc region.

The Fc variants of the present invention may be combined with other Fc modifications, including but not limited to modifications that alter effector function or interaction with one or more Fc ligands. Such combination may provide additive, synergistic, or novel properties in antibodies or Fc fusions. In one embodiment, the Fc variants of the present invention may be combined with other known Fc variants (Duncan et al., 1988, Nature 332:563-564; Lund et al., 1991, J Immunol 147:2657-2662; Lund et al., 1992, Mol Immunol 29:53-59; Alegre et al., 1994, Transplantation 57:1537-1543; Hutchins et al., 1995, Proc Natl Acad Sci USA 92:11980-11984; Jefferis et al., 1995, Immunol Left 44:111-117; Lund et al., 1995, Faseb J9:115-119; Jefferis et al., 1996, Immunol Left 54:101-104; Lund et al., 1996, J Immunol 157:4963-4969; Armour et al., 1999, Eur J Immunol 29:2613-2624; Idusogie et al., 2000, J Immunol 164:4178-4184; Reddy et al., 2000, J Immunol 164:1925-1933; Xu et al., 2000, Cell Immunol 200:16-26; Idusogie et al., 2001, J Immunol 166:2571-2575; Shields et al., 2001, J Biol Chem 276:6591-6604; Jefferis et al., 2002, Immunol Left 82:57-65; Presta et al., 2002, Biochem Soc Trans 30:487-490; Hinton et al., 2004, J Biol Chem 279:6213-6216) (U.S. Pat. No. 5,624,821; U.S. Pat. No. 5,885,573; U.S. Pat. No. 6,194,551; PCT WO 00/42072; PCT WO 99/58572; US 2004/0002587 A1). In an alternate embodiment, the Fc variants of the present invention are incorporated into an antibody or Fc fusion that comprises one or more engineered glycoforms (infra). Thus combinations of the Fc variants of the present invention with other Fc modifications, as well as undiscovered Fc modifications, are contemplated with the goal of generating novel antibodies or Fc fusions with optimized properties.

B. Anti-Notch1 Engineered Glycoforms

The invention additionally, encompasses anti-antibodies including fragments thereof which are differentially modified during or after translation, e.g., by glycosylation, proteolytic cleavage etc. Any of numerous chemical modifications may be carried out by known techniques, including but not limited to, specific chemical cleavage by trypsin, papain, metabolic synthesis in the presence of tunicamycin etc.

Antibodies are glycoproteins containing carbohydrate structures at conserved positions in the heavy chain constant regions, with each isotype possessing a distinct array of N-linked carbohydrate structures, which variably affect protein assembly, secretion or functional activity. The structure of the attached N-linked carbohydrate varies considerably and can include high-mannose, multiply-branched as well as biantennary complex oligosaccharides. (Wright, A., and Morrison, S. L., Trends Biotech. 15:26-32 (1997)). The major carbohydrate units are attached to amino acid residues of the constant region of the antibody. Carbohydrate is also known to attach to the antigen binding sites of some antibodies and may affect the antibody-binding characteristics by limiting access of the antigen to the antibody binding site. Typically, there is heterogeneous processing of the core oligosaccharide structures attached at a particular glycosylation site such that even monoclonal antibodies exist as multiple glycoforms. Likewise, it has been shown that major differences in antibody glycosylation occur between cell lines, and even minor differences are seen for a given cell line grown under different culture conditions. (Lifely, M. R. et al., Glycobiology 5(8):813-22 (1995)).

Monoclonal antibodies often achieve their therapeutic benefit through two binding events. First, the variable domain of the antibody binds to a specific tumor receptor on the surface of the target cell. This is followed by recruitment of effector cells such as natural killer (NK) cells that bind to the constant region (Fc) of the antibody and destroy cells to which the antibody has bound. This process, known as antibody-dependent cell cytotoxicity (ADCC), partially depends on a specific N-glycosylation event at Asn 297 in the Fc domain of the heavy chain of IgG1s. In general, antibodies that lack this N-glycosylation structure still bind antigen but cannot mediate ADCC, apparently as a result of reduced affinity of the Fc domain of the antibody for the Fc receptor FcγRIIIa on the surface of NK cells. Interestingly, there is a linear increase of in vitro complement activation with increasing terminal galactosylation of the carbohydrate moiety in the Fc domain. There are a number of roles associated with the carbohydrate units. Glycosylation may affect overall solubility and the rate of catabolism of the antibody. It is also known that carbohydrate is necessary for cellular secretion of some antibody chains. It has been demonstrated that glycosylation of the constant region plays a vital role in the effector functioning of an antibody; without this glycosylation in its correct configuration, the antibody may be able to bind to the antigen but may not be able to bind for example to macrophages, helper and suppressor cells or complement, to carry out its role of blocking or lysing the cell to which it is bound. Hyperglycosylated proteins have been shown to exhibit increased serum half-life, are less sensitive to proteolysis and more heat-stable compared with the non-glycosylated forms. (Leatherbarrow et al., Mol. Immunol. 22:407 (1985)).

IgG1 type antibodies, which represent the most commonly used antibodies in cancer immunotherapy, are glycoproteins that have a conserved N-linked glycosylation site at Asn297 in each CH2 domain. The two complex bi-antennary oligosaccharides attached to Asn297 are buried between the CH2 domains, forming extensive contacts with the polypeptide backbone, and their presence is essential for the antibody to mediate effector functions such as antibody dependent cellular cytotoxicity (ADCC) (Lifely, M. R., et al., Glycobiology 5:813-822 (1995); Jefferis, R., et al., Immunol Rev. 163:59-76 (1998); Wright, A. and Morrison, S. L., Trends Biotechnol. 15:26-32 (1997)). Glycosylation of IgG at asparagine 297 in the C_{H}2 domain is also required for full capacity of IgG to activate the classical pathway of complement-dependent cytolysis (Tao and Morrison, J. Immunol. 143:2595 (1989)).

More, glycosylation of IgM at asparagine 402 in the CH 3 domain is necessary for proper assembly and cytolytic activity of the antibody (Muraoka and Shulman, J. Immunol. 142:695 (1989)). Likewise, removal of glycosylation sites as positions 162 and 419 in the CH1 and CH3 domain of an IgA antibody has been shown to lead to intracellular degradation and at least 90% inhibition of secretion (Taylor and Wall, Mol. Cell. Biol. 8:4197 (1988)).

Glycosylation of immunoglobulins in the variable (V) region has also been observed. Sox and Hood, Proc. Natl. Acad. Sci. USA 66:975 (1970), reported that about 20% of human antibodies are glycosylated in the V region. Glycosylation of the V domain is believed to arise from fortuitous occurrences of the N-linked glycosylation signal Asn-Xaa-Ser/Thr in the V region sequence and has not been recognized in the art as playing an important role in immunoglobulin function.

It has also been reported that glycosylation at CDR2 of the heavy chain, in the antigen binding site, of a murine antibody specific for α-(1-6)dextran increases its affinity for dextran (Wallick et al., J. Exp. Med. 168:1099 (1988) and Wright et al., EMBO J. 10:2717 (1991)). See U.S. Pat. No. 6,933,368. Some classes and subclasses also have O-linked sugars, often in the hinge region, e.g. IgD and IgA from some species.

For example, the absence of fucose or the presence of a bisecting N-acetylglucosamine in the carbohydrate structure of the monoclonal antibody, has been positively correlated with the potency of ADCC. Specifically, defucosylated carbohydrate residues on monoclonal antibodies have been shown to enhance the ADCC capability of the target antibody more than threefold. “Glycosylation of therapeutic proteins in different production systems” Acta Paediatrica, 96: 17-22 (2007); Shields et al., “Lack of Fucose on Human IgG1 N-Linked Oligosaccharide Improves Binding to Human FcγRIII and Antibody-dependent Cellular Toxicity”, J. Biol. Chem., 277: 26733-26740 (2002). Likewise, specific engineered glycoforms of monoclonal antibodies, which interact solely with the FcγRIIIa receptor of natural killer cells, exhibit superior ADCC compared with heterogeneous glycoforms that interact with different Fc receptors. The collective data impel the conclusion that glycoengineering for directed glycosylation of therapeutic proteins can improve the therapeutic effect in vivo. See Umaa, P. et al., Nature Biotechnol. 17:176-180 (1999)). See also U.S. Pat. No. 5,624,821; U.S. Pat. No. 6,602,684; WO 00/42072 and WO 07/048122, the content of each of which is incorporated in its entirety by reference herein. See also US serial No. 2006/0182744. Not only is ADCC dependent on glycosylation of the Fc domain, but the degree of cell-mediated killing is also sensitive to the composition of the glycans in the Fc region of the antibody.

As a consequence, the present invention, in related embodiments, provides “Engineered Glycoforms” of any one or more of the anti-antibodies disclosed herein including fragments thereof, wherein the glycosylation profiles of the antibody are altered to enhance their use in the treatment of specific types of cancers or other disease states.

By “engineered glycoform” as used herein is meant a carbohydrate composition that is covalently attached to an Fc polypeptide, wherein the carbohydrate composition differs chemically from that of a parent Fc polypeptide. Engineered glycoforms may be useful for a variety of purposes, including but not limited to enhancing or reducing effector function. Engineered glycoforms may be generated by a variety of methods known in the art (Umana et al., 1999, Nat Biotechnol 17:176-180; Davies et al., 2001, Biotechnol Bioeng 74:288-294; Shields et al., 2002, J Biol Chem 277:26733-26740; Shinkawa et al., 2003, J Biol Chem 278:3466-3473); (U.S. Pat. No. 6,602,684; U.S. Ser. No. 10/277,370; U.S. Ser. No. 10/113,929; PCT WO 00/61739A1; PCT WO 01/29246A1; PCT WO 02/31140A1; PCT WO 02/30954A1); (Potelligent™ technology [Biowa, Inc., Princeton, N.J.]; GlycoMAb™ glycosylation engineering technology [GLYCART biotechnology AG, Zurich, Switzerland)). Many of these techniques are based on controlling the level of fucosylated and/or bisecting oligosaccharides that are covalently attached to the Fc region, for example by expressing an Fc polypeptide in various organisms or cell lines, engineered or otherwise (for example Lec-13 CHO cells or rat hybridoma YB2/0 cells), by regulating enzymes involved in the glycosylation pathway (for example FUT8 [α1,6-fucosyltranserase] and/or β1-4-N-acetylglucosaminyltransferase III [GnTIII]), or by modifying carbohydrate(s) after the Fc polypeptide has been expressed. Engineered glycoform typically refers to the different carbohydrate or oligosaccharide; thus an Fc polypeptide, for example an antibody or Fc fusion, may comprise an engineered glycoform. Alternatively, engineered glycoform may refer to the Fc polypeptide that comprises the different carbohydrate or oligosaccharide.

Covalent modification of the target antibody included within the scope of this invention comprises altering the native glycosylation pattern of the polypeptide. “Altering the native glycosylation pattern” is intended for purposes herein to mean deleting one or more carbohydrate moieties found in the native target antibody, and/or adding one or more glycosylation sites that are not present in the native target antibody.

In still another embodiment, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, EP 1,176,195 by Hanai et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17:176-180).

Antibodies disclosed herein can be glycosylated in both the C-regions and in the V-regions. Obviously C-region glycosylation is dependent on the particular sequence which by definition defines the class and subclass of the antibody. As noted elsewhere, many classes of antibody have conserved N-linked glycosylation sites in the constant domains. For example all IgG antibodies have a conserved N-linked glycosylation site in the CH2 domain at residue Asn297.

Two basic types of glycosylation of therapeutic proteins are known: O-linked and N-linked glycosylation. O-linked glycosylation is initiated by the attachment of N-acetyl-galactosamine to a serine or threonine residue in the peptide backbone of the therapeutic protein. The proximal carbohydrate is the target for glycosyltransferases to form a mature O-glycan structure. It is difficult to predict where O-linked glycosylation will occur in the protein as there is no clear consensus amino acid sequence for O-linked glycosylation (3, 4). However, O-linked glycosylation is affected by secondary structural elements such as an extended β-turn. In contrast, consensus amino acid sequences are known for N-glycosylation. N-glycosylation occurs at a specific sequence motif, Asn-X-Thr/Ser (sequon or consensus sequence; where X is any amino acid except proline), and this consensus sequence must be accessible to the precursor transferring the enzyme. In the case where X=Pro, no glycosylation takes place. Asn-X-Thr/Ser sequences in β-turns can influence the protein conformation by N-linked glycosylation. As glycosylation precedes final protein folding, the structure of the resultant therapeutic protein may be altered, resulting in differences in activity or stability compared with the non-glycosylated form. Thus, recombinant antibodies of the invention can be modified to recreate or create additional glycosylation sites if desired, which is simply achieved by engineering the appropriate amino acid sequences (such as Asn-X-Ser, Asn-X-Thr, Ser, or Thr) into the primary sequence of the antibody. Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

Thus, in certain embodiments of the invention a mutant anti-antibody is provided that exhibits a higher affinity for its antigen e.g., receptor or endogenous binding partner, than a parent antibody that comprises a parent immunoglobulin chain, wherein the mutant immunoglobulin chain comprises an amino acid substitution that eliminates a variable region glycosylation site of the parent immunoglobulin chain, said elimination having the effect of increasing the affinity of the mutant antibody relative to the parent antibody. Alternative embodiments contemplate variants that are “aglycosylated.”

“Glycosylation sites” refer to amino acid residues which are recognized by a eukaryotic cell as locations for the attachment of sugar residues. The amino acids where carbohydrate, such as oligosaccharide, is attached are typically asparagine (N-linkage), serine (O-linkage), and threonine (O-linkage) residues. In order to identify potential glycosylation sites within an antibody or antigen-binding fragment, the sequence of the antibody is examined, for example, by using publicly available databases such as the website provided by the Center for Biological Sequence Analysis (see http://www.cbs.dtu.dk/services/NetNGlyc/ for predicting N-linked glycoslyation sites) and http://www.cbs.dtu.dk/services/NetOGlyc/ for predicting O-linked glycoslyation sites). Additional methods for altering glycosylation sites of antibodies are described in U.S. Pat. Nos. 6,350,861 and 5,714,350.

Several approaches have been attempted to alter the glycosylation state of IgG antibodies: inhibition of glycosylation by culturing cells in the presence of the drug tunicamycin (Leatherbarrow et al. 1985; Walker et al. 1989; Pound et al. 1993); treatment of glycoproteins with specific glycosidases that remove the entire oligosaccharide or specific residues (Tsuchiya et al. 1989; Boyd et al. 1995); or site-directed mutagenesis to remove either the carbohydrate addition site (Tao, Smith et al. 1993) or residues within the CH2 region that contact the core oligosaccharide residues (Lund, Takahashi et al. 1996). These studies have confirmed that the presence of carbohydrate is essential to antibody function.

Glycosylation can be achieved by methods known in the art, e.g., by producing the antibody in a mammalian host cell such as Chinese Hamster Ovary (CHO) cell or in yeast. Addition of glycosylation sites to the target antibody is conveniently accomplished by altering the amino acid sequence such that it contains one or more of the above-described tri-peptide sequences (for N-linked glycosylation sites). The alteration may also be made by the addition of, or substitution by, one or more serine or threonine residues to the native target antibody sequence (for O-linked glycosylation sites). For ease, the target antibody amino acid sequence is preferably altered through changes at the DNA level, particularly by mutating the DNA encoding the target antibody at preselected bases such that codons are generated that will translate into the desired amino acids. The DNA mutation(s) may be made using methods described above under the heading of “Amino Acid Sequence Variants of Target antibody”.

Yeast provides substantial advantages over bacteria for the production of immunoglobulin H and L chains. Yeasts carry out post-translational peptide modifications including glycosylation. A number of recombinant DNA strategies now exist which utilize strong promoter sequences and high copy number plasmids which can be used for production of the desired proteins in yeast. Yeast recognizes leader sequences of cloned mammalian gene products and secretes peptides bearing leader sequences (i.e., pre-peptides) (Hitzman, et al., 11th International Conference on Yeast, Genetics and Molecular Biology, Montpelier, France, Sep. 13-17, 1982).

Another means of increasing the number of carbohydrate moieties on the target antibody is by chemical or enzymatic coupling of glycosides to the polypeptide. These procedures are advantageous in that they do not require production of the polypeptide in a host cell that has glycosylation capabilities for N-and O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330 published Sep. 11, 1987, and in Aplin and Wriston (CRC Crit. Rev. Biochem., pp. 259-306 [1981]).

Moreover, the main species antibody or variant thereof may further comprise glycosylation variations, non-limiting examples of which include antibody comprising a G1 or G2 oligosaccharide structure attached to the Fc region thereof, antibody comprising a carbohydrate moiety attached to a light chain thereof (e.g. one or two carbohydrate moieties, such as glucose or galactose, attached to one or two light chains of the antibody, for instance attached to one or more lysine residues), antibody comprising one or two non-glycosylated heavy chains, or antibody comprising a sialidated oligosaccharide attached to one or two heavy chains thereof etc.

Immune effector functions are unnecessary or even deleterious in certain clinical settings. In another embodiment of the invention, antibodies or fragments thereof are altered wherein the constant region of the antibody is modified to reduce at least one constant region-mediated biological effector function relative to an unmodified antibody. Such modified antibodies are often referred to as “aglycosylated” antibodies. In order to improve the binding affinity of an antibody of the invention or antigen-binding fragment thereof to the antigen while minimizing or reducing its binding to the Fc receptor, the antibody can be mutated at particular regions necessary for Fc receptor (FcR) interactions (see e.g., Canfield, S. M. and S. L. Morrison (1991) J. Exp. Med. 173:1483-1491; and Lund, J. et al. (1991) J. of Immunol. 147:2657-2662). Reduction in FcR binding ability of the antibody may also reduce other effector functions which rely on FcR interactions, such as opsonization and phagocytosis and antigen-dependent cellular cytotoxicity, glycosylation sites of the antibody can be altered, for example, by mutagenesis (e.g., site-directed mutagenesis). As a consequence, such antibodies do not exhibit substantial immune effector functions that are dependent on glycosylation of the Fc region. Generally and preferably, an aglycosylated antibody of the invention does not exhibit substantial immune effector functions except for binding to FcRn. In some embodiments, an antibody of the invention or a fragment thereof does not possess substantial or completely lacks effector functions other than FcRn binding. In one embodiment, said effector function is complement lysis. In one embodiment, said effector function is antibody dependent cell cytotoxicity (ADCC). In one embodiment, the antibody fragment binds FcRn.

Aglycosylated antibodies can be produced by a variety of methods known in the art. A convenient method comprises expressing the antibody in a prokaryotic host cell such as E. coli. The amino acids where carbohydrate, such as oligosaccharide, is attached are typically asparagine (N-linkage), serine (O-linkage), and threonine (O-linkage) residues. In order to identify potential glycosylation sites within an antibody or antigen-binding fragment, the sequence of the antibody is examined, for example, by using publicly available databases such as the website provided by the Center for Biological Sequence Analysis (see http://www.cbs.dtu.dk/services/NetNGlyc/ for predicting N-linked glycoslyation sites) and http://www.cbs.dtu.dk/services/NetOGlyc/ for predicting O-linked glycoslyation sites). Additional methods for altering glycosylation sites of antibodies are described in U.S. Pat. Nos. 6,350,861 and 5,714,350.

Removal of carbohydrate moieties present on the native target antibody may also be accomplished chemically or enzymatically. Chemical deglycosylation requires exposure of the polypeptide to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al. (Arch. Biochem. Biophys., 259:52 [1987]) and by Edge et al. (Anal. Biochem., 118:131 [1981]). Enzymatic cleavage of carbohydrate moieties on polypeptides can be achieved by the use of a variety of endo- and exo-glycosidases as described by Thotakura et al. (Meth. Enzymol. 138:350 [1987]). Glycosylation at potential glycosylation sites may be prevented by the use of the compound tunicamycin as described by Duskin et al. (J. Biol. Chem., 257:3105 [1982]). Tunicamycin blocks the formation of protein-N-glycoside linkages.

Thus, in some embodiments, the antibodies of the invention or an antigen-binding fragment thereof is modified to reduce or eliminate potential glycosylation sites. In still another embodiment, the constant region of the antibody, or fragment thereof of the invention is modified to reduce at least one constant region-mediated biological effector function relative to an unmodified antibody.

### Antibody Derivatives

The antibodies of the present invention can be further modified to contain additional nonproteinaceous moieties that are known in the art and readily available. Preferably, the moieties suitable for derivatization of the antibody are water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymers are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

Other modifications of the antibody are contemplated herein. For example, the antibody may be linked to one of a variety of nonproteinaceous polymers, e.g., polyethylene glycol, polypropylene glycol, polyoxyalkylenes, or copolymers of polyethylene glycol and polypropylene glycol. The antibody also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980). In Vivo Stabilization Using Polymeric Stabilizing Moieties—PEGylation:

Another type of covalent modification of the target antibody, e.g., any one or more of the anti-Notch1 antibodies of the invention comprises linking the target antibody to various nonproteinaceous polymers, e.g. polyethylene glycol, polypropylene glycol or polyoxyalkylenes, in the manner set forth in U.S. Pat. Nos. 4,640,835; 4,496,689; 4,301,144; 4,670,417; 4,791,192 or 4,179,337. Thus, in some embodiments, the antibodies and antibody fragments of the invention may be chemically modified to provide a desired effect such as increased solubility, stability and circulating time of the polypeptide, or decreased immunogenicity.

The antibody or fragments thereof polypeptides may be modified at random positions within the molecule, or at predetermined positions within the molecule and may include one, two, three or more attached chemical moieties. For example, PEGylation of antibodies and antibody fragments of the invention may be carried out by any of the PEGylation reactions known in the art, See for example, EP 0 154 316 by Nishimura et al. and EP 0 401 384 by Ishikawa et al. Methods for determining the degree of substitution are discussed, for example, in Delgado et al., Crit. Rev. Thera. Drug Carrier Sys. 9:249-304 (1992). (each of which is incorporated by reference herein in its entirety). To PEGylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the PEGylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be PEGylated is an aglycosylated antibody

In another aspect, a single immunoglobulin variable domain derived from an invention antibody containing composition is stabilized in vivo by linkage or association with a (non-polypeptide) polymeric stabilizing moiety. Examples of this type of stabilization are described, for example, in WO99/64460 (Chapman et al.) and EP1,160,255 (King et al.), each of which is incorporated herein by reference. Specifically, these references describe the use of synthetic or naturally-occurring polymer molecules, such as polyalkylene, polyalkenylenes, polyoxyalkylenes or polysaccharides, to increase the in vivo half-life of immunoglobulin polypeptides. A typical example of a stabilizing moiety is polyethylene glycol, or PEG, a polyalkylene. The process of linking PEG to an immunoglobulin polypeptide is described in these references and is referred to herein as “PEGylation.” As described therein, an immunoglobulin polypeptide can be PEGylated randomly, as by attachment of PEG to lysine or other amino acids on the surface of the protein, or site-specifically, e.g., through PEG attachment to an artificially introduced surface cysteine residue. Depending upon the immunoglobulin, it may be preferred to use a non-random method of polymer attachment, because random attachment, by attaching in or near the antigen-binding site or sites on the molecule often alters the affinity or specificity of the molecule for its target antigen. Polyethylene glycol can also be attached to proteins using a number of different intervening linkers. For example, U.S. Pat. No. 5,612,460, the entire disclosure of which is incorporated herein by reference, discloses urethane linkers for connecting polyethylene glycol to proteins. Protein-polyethylene glycol conjugates wherein the polyethylene glycol is attached to the protein by a linker can also be produced by reaction of proteins with compounds such as MPEG-succinimidylsuccinate, MPEG activated with 1,1′-carbonyldiimidazole, MPEG-2,4,5-trichloropenylcarbonate, MPEG-p-nitrophenolcarbonate, and various MPEG-succinate derivatives. A number of additional polyethylene glycol derivatives and reaction chemistries for attaching polyethylene glycol to proteins are described in WO 98/32466, the entire disclosure of which is incorporated herein by reference. PEGylated protein products produced using the reaction chemistries set out herein are included within the scope of the invention.

One system for attaching polyethylene glycol directly to amino acid residues of proteins without an intervening linker employs tresylated MPEG, which is produced by the modification of monmethoxy polyethylene glycol (MPEG) using tresylchloride (ClSO₂CH₂CF₃). Upon reaction of protein with tresylated MPEG, polyethylene glycol is directly attached to amine groups of the protein. Thus, the invention includes protein-polyethylene glycol conjugates produced by reacting proteins of the invention with a polyethylene glycol molecule having a 2,2,2-trifluoroethane sulphonyl group.

A general method for preparing PEGylated antibodies and antibody fragments of the invention will generally comprise the steps of (a) reacting the antibody or antibody fragment with polyethylene glycol, such as a reactive ester or aldehyde derivative of PEG, under conditions whereby the antibody or antibody fragment becomes attached to one or more PEG groups, and (b) obtaining the reaction products. It will be apparent to one of ordinary skill in the art to select the optimal reaction conditions or the acylation reactions based on known parameters and the desired result.

There are a number of attachment methods available to those skilled in the art, e.g., EP 0 401 384, herein incorporated by reference (coupling PEG to G-CSF), see also Malik et al., Exp. Hematol. 20:1028-1035 (1992) (reporting PEGylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues, glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

As suggested above, polyethylene glycol may be attached to proteins via linkage to any of a number of amino acid residues. As indicated above, pegylation of the proteins of the invention may be accomplished by any number of means. For example, polyethylene glycol may be attached to the protein either directly or by an intervening linker. Linkerless systems for attaching polyethylene glycol to proteins are described in Delgado et al., Crit. Rev. Thera. Drug Carrier Sys. 9:249-304 (1992); Francis et al., Intern. J. of Hematol. 68:1-18 (1998); U.S. Pat. No. 4,002,531; U.S. Pat. No. 5,349,052; WO 95/06058; and WO 98/32466, the disclosures of each of which are incorporated herein by reference. For example, polyethylene glycol can be linked to a protein via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) of the protein or to more than one type of amino acid residue (e.g., lysine, histidine, aspartic acid, glutamic acid, cysteine and combinations thereof) of the protein.

One may specifically desire proteins chemically modified at the N-terminus. Using polyethylene glycol as an illustration of the present composition, one may select from a variety of polyethylene glycol molecules (by molecular weight, branching, etc.), the proportion of polyethylene glycol molecules to protein (or peptide) molecules in the reaction mix, the type of PEGylation reaction to be performed, and the method of obtaining the selected N-terminally PEGylated protein. The method of obtaining the N-terminally PEGylated preparation (i.e., separating this moiety from other mono-PEGylated moieties if necessary) may be by purification of the N-terminally PEGylated material from a population of PEGylated protein molecules. Selective proteins chemically modified at the N-terminus may be accomplished by reductive alkylation which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

The polymer may be of any molecular weight, and may be branched or unbranched. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750(1999); and Caliceti et al., Bioconjug. Chem. 10:638-646 (1999), the disclosures of each of which are incorporated herein by reference. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

It is preferred that the addition of PEG or another polymer does not interfere with the antigen-binding affinity or specificity of the antibody variable domain polypeptide. By “does not interfere with the antigen-binding affinity or specificity” is meant that the PEG-linked antibody single variable domain has an IC50 or ND50 which is no more than 10% greater than the IC50 or ND50, respectively, of a non-PEG-linked antibody variable domain having the same antibody single variable domain. In the alternative, the phrase “does not interfere with the antigen-binding affinity or specificity” means that the PEG-linked form of an antibody single variable domain retains at least 90% of the antigen binding activity of the non-PEGylated form of the polypeptide.

PEGylated antibodies and antibody fragments may generally be used to treat conditions that may be alleviated or modulated by administration of the antibodies and antibody fragments described herein. Generally the PEGylated antibodies and antibody fragments have increased half-life, as compared to the non-PEGylated antibodies and antibody fragments. The PEGylated antibodies and antibody fragments may be employed alone, together, or in combination with other pharmaceutical compositions.

The target antibody may also be entrapped in microcapsules prepared, for example, by coacervation techniques or by interfacial polymerization (for example, hydroxymethylcellulose or gelatin-microcapsules and poly-[methylmethacylate]microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Osol, A., Ed., (1980).

### Antibody-Coated Liposomes and Therapeutics (Immunoliposomes)

Liposomal formulations are often used in therapeutics and pharmaceuticals. However, the biodistribution of liposomes in initial studies meant that such formulations were not widely applicable for use in humans. The technology of “stealth or stealthed” liposomes and formulations was thus developed, which allows liposomes to circulate for longer durations??. A preferred agent for use in stealthing liposomes is polyethylene glycol (PEG), and the resultant liposomes are also termed PEGylated liposomes.

Any one of the antibodies or fragments thereof disclosed herein may also be formulated as immunoliposomes. Liposomes containing the antibody are prepared by methods known in the art, such as described in Epstein et al., Proc. Natl. Acad. Sci. USA 82: 3688 (1985); Hwang et al., Proc. Natl. Acad Sci. USA 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem. 257: 286-288 (1982) via a disulfide interchange reaction. A chemotherapeutic agent (such as Doxorubicin) is optionally contained within the liposome. See Gabizon et al., J. National Cancer Inst. 81 (19): 1484 (1989).

Stealth liposomes have also been proposed for use in delivering cytotoxic agents to tumors in cancer patients. A range of drugs have been incorporated into stealth liposomes, including cisplatin (Rosenthal et al., 2002), TNFα (Kim et al., 2002), doxorubicin (Symon et al., 1999) and adriamycin (Singh et al., 1999), each reference being specifically incorporated herein by reference. However, recent reports have indicated unexpected low efficacy of stealth liposomal doxorubicin and vinorelbine in the treatment of metastatic breast cancer (Rimassa et al., 2003). See also U.S. Patent Application No. 20040170620, the content of which is incorporated in entirety by reference herein.

Thus, in certain embodiments, the invention provides improved stealthed liposome formulations, in which the stealthed liposomes are functionally associated or “coated” with an antibody that binds to an aminophospholipid or anionic phospholipid, preferably to PS or PE. The 9D2, 3G4 (ATCC 4545) and like, competing antibodies of the invention are preferred for such uses, although any antibody, or antigen binding region thereof, which binds to an aminophospholipid or anionic phospholipid may be used.

Any stealthed liposome may form the basis of the new liposomal formulations, and preferably a PEGylated liposome will be employed. The stealthed liposomes are “coated”, i.e., operatively or functionally associated with the antibody that binds to an aminophospholipid or anionic phospholipid. The operative or functional association is made such that the antibody retains the ability to specifically bind to the target aminophospholipid or anionic phospholipid, preferably PS or PE, thereby delivering or targeting the stealthed liposome and any contents thereof to PS- and/or PE-positive cells, such as tumor cells and tumor vascular endothelial cells.

The antibody-coated stealthed liposomes of the invention may be used alone. Preferably, however, such liposomes will also contain one or more second therapeutic agents, such as anti-cancer or chemotherapeutic agents (the first therapeutic agent being the antibody itself). The second therapeutic agents are generally described as being within the “core” of the liposome. Any one or more of the second, anti-cancer or chemotherapeutic agents known in the art and/or described herein for conjugation to antibodies, or for combination therapies, may be used in the antibody-coated stealthed liposomes of the invention, for example, any chemotherapeutic or radiotherapeutic agent, cytokine, anti-angiogenic agent or apoptosis-inducing agent. In certain embodiments, preferred chemotherapeutic agents are anti-tubulin drugs, docetaxel and paclitaxel.

Moreover, the antibody-coated stealthed liposomes of the invention may also be loaded with one or more anti-viral drugs for use in treating viral infections and diseases. As with the anti-cancer agents, any one or more of the second, anti-viral drugs known in the art and/or described herein for conjugation to antibodies, or for combination therapies, may be used in the antibody-coated stealthed liposomes of the invention.

In other embodiments of the invention the invention antibodies or antigen-binding fragments thereof are conjugated to albumen using art recognized techniques.

To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fe region of an IgG molecule (e.g., IgG₁, IgG₂, IgG₃, or IgG₄) that is responsible for increasing the in vivo serum half-life of the IgG molecule.

### Species and Molecule Selectivity

The anti-Notch1 antibody of the invention including binding fragments thereof demonstrates both species and molecule selectivity. In one aspect, the anti-Notch1 antibody of the invention binds to human Notch1. Following the teachings of the specification, one may determine the species selectivity for the anti-Notch1 antibody using methods well known in the art. For instance, one may determine species selectivity using Western blot, FACS, ELISA or RIA. In a preferred embodiment, one may determine the species selectivity using Western blot.

Likewise, one may determine the selectivity of an anti-Notch1 antibody for Notch1 using methods well known in the art following the teachings of the specification. For instance, one may determine the molecule selectivity using Western blot, FACS, ELISA or RIA. In a preferred embodiment, one may determine the molecular selectivity using Western blot.

### Naked Antibody Therapy

A therapeutically effective amount of a naked fully human anti-Notch1 antibody, or fragments thereof can be formulated in a pharmaceutically acceptable excipient. The efficacy of the naked fully human Notch1 antibodies and their fragments can also be enhanced by supplementing these naked antibodies with one or more other naked antibodies, with one or more immunoconjugates of fully human invention Notch1 antibodies, conjugated with one or more therapeutic agents, including drugs, toxins, immunomodulators, hormones, oligonucleotides, hormone antagonists, enzymes, enzyme inhibitors, therapeutic radionuclides, an angiogenesis inhibitor, etc., administered concurrently or sequentially or according to a prescribed dosing regimen, with the Notch1 antibodies or fragments thereof.

### Antibody Dependent Enzyme Mediated Prodrug Therapy (ADEPT)

The antibody of the present invention may also be used in ADEPT by conjugating the antibody to a prodrug-activating enzyme which converts a prodrug (e.g., a peptidyl chemotherapeutic agent, see WO81/01145) to an active anti-cancer drug. See, for example, WO 88/07378 and U.S. Pat. No.4,975,278.

The enzyme component of the immunoconjugate useful for ADEPT includes any enzyme capable of acting on a prodrug in such a way so as to convert it into its more active, cytotoxic form.

Enzymes that are useful in the method of this invention include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxylpeptidascs, useful for converting prodrugs that contain D-amino acid substituents; carbohydrate-cleaving enzymes such as beta-galactosidase and neuraminidase useful for converting glycosylated prodrugs into free drugs; beta-lactamase useful for converting drugs derivatized with beta-lactams into free drugs; and penicillin amidases, such as penicillin V amidase or penicillin G amidase, useful for converting drugs derivatized at their amine nitrogens with phenoxylacetyl or phenylacetyl groups, respectively, into free drugs. Alternatively, antibodies with enzymatic activity, also known in the art as “abzymes”, can be used to convert to prodrugs of the invention into free active drugs (Massey, Nature 328: 457-458 (1987)). Antibody-abzyme conjugates can be prepared as described herein for delivery of the abzyme to a tumor cell population.

The enzymes of this invention can be covalently bound to the antibody mutant by techniques well known in the art such as the use of the heterobifunctional crosslinking reagents discussed above. Alternatively, fusion proteins comprising at least the antigen binding region of an antibody of the invention linked to at least a functionally active portion of an enzyme of the invention can be constructed using recombinant DNA techniques well known in the art (Neubergeret et al., Nature 312: 604-608(1984)).

Antibodies with enzymatic activity, known as catalytic antibodies or “abzymes”, can also be employed to convert prodrugs into active drugs. Abzymes based upon the antibodies of the invention, preferably the 9D2 and 3G4 and like antibodies, thus form another aspect of the present invention. The technical capacity to make abzymes also exists within those of ordinary skill in the art, as exemplified by Massey et al. (1987), specifically incorporated herein by reference for purposes of supplementing the abzyme teaching. Catalytic antibodies capable of catalyzing the breakdown of a prodrug at the carbamate position, such as a nitrogen mustard aryl carbamate, are further contemplated, as described in EP 745,673, specifically incorporated herein by reference.

Screening for Antibodies with Desired Properties

Techniques for generating antibodies have been described above. The antibodies of the present invention can be characterized for their physical/chemical properties and biological functions by various assays known in the art. In some embodiments, antibodies are characterized for any one or more of binding to Notch1 receptor protein, and/or reduction or blocking of Notch receptor activation; and/or reduction or blocking of Notch receptor downstream molecular signaling; and/or disruption or blocking of Notch receptor binding to its native ligand, e.g, serrate or delta etc; and/or promotion of endothelial cell proliferation; and/or inhibition of endothelial cell differentiation; and/or inhibition of arterial differentiation; and/or inhibition of tumor vascular perfusion; and/or treatment and/or prevention of a tumor, cell proliferative disorder or a cancer; and/or treatment or prevention of a disorder associated with Notch1 expression and/or activity; and/or treatment or prevention of a disorder associated with Notch receptor expression and/or activity.

In certain embodiments, antibodies may be selected based upon certain biological characteristics such as for example assessing the growth inhibitory effects of an anti-Notch1 antibody of the invention. This property may be assessed by methods known in the art, e.g., using cells which express Notch receptor either endogenously or following transfection with the Notch receptor gene. For example, tumor cell lines and Notch receptor-transfected cells may be treated with an anti-Notch receptor monoclonal antibody of the invention at various concentrations for a few days (e.g., 2-7) days and stained with crystal violet or MTT or analyzed by some other colorimetric assay. Another method of measuring proliferation would be by comparing ³H-thymidine uptake by the cells treated in the presence or absence of an anti-Notch receptor antibody of the invention. After antibody treatment, the cells are harvested and the amount of radioactivity incorporated into the DNA quantitated in a scintillation counter. Appropriate positive controls include treatment of a selected cell line with a growth inhibitory antibody known to inhibit growth of that cell line. Preferably, the Notch receptor agonist will inhibit cell proliferation of a Notch receptor-expressing tumor cell in vitro or in vivo by about 25-100% compared to the untreated tumor cell, more preferably, by about 30-100%, and even more preferably by about 50-100% or 70-100%, at an antibody concentration of about 0.5 to 30 μg/ml. Growth inhibition can be measured at an antibody concentration of about 0.5 to 30 μg/ml or about 0.5 nM to 200 nM in cell culture, where the growth inhibition is determined 1-10 days after exposure of the tumor cells to the antibody. The antibody is growth inhibitory in vivo if administration of the anti-Notch receptor antibody at about 1 μg/kg to about 100 mg/kg body weight results in reduction in tumor size or tumor cell proliferation within about 5 days to 3 months from the first administration of the antibody.

The purified antibodies can be further characterized by a series of assays including, but not limited to, N-terminal sequencing, amino acid analysis, non-denaturing size exclusion high pressure liquid chromatography (HPLC), mass spectrometry, ion exchange chromatography and papain digestion.

The assaying method for detecting Notch1 using the antibodies of the invention or binding fragments thereof are not particularly limited. Any assaying method can be used, so long as the amount of antibody, antigen or antibody-antigen complex corresponding to the amount of antigen (e.g., the level of Notch1) in a fluid to be tested can be detected by chemical or physical means and the amount of the antigen can be calculated from a standard curve prepared from standard solutions containing known amounts of the antigen. Representative immunoassays encompassed by the present invention include, but are not limited to, those described in U.S. Pat. No. 4,367,110 (double monoclonal antibody sandwich assay); Wide et al., Kirkham and Hunter, eds. Radioimmunoassay Methods, E. and S. Livingstone, Edinburgh (1970); U.S. Pat. No. 4,452,901 (western blot); Brown et al., J. Biol. Chem. 255: 4980-4983 (1980) (immunoprecipitation of labeled ligand); and Brooks et al., Clin. Exp. Immunol. 39:477 (1980) (immunocytochemistry); immunofluorescence techniques employing a fluorescently labeled antibody, coupled with light microscopic, flow cytometric, or fluorometric detection etc. See also Immunoassays for the 80's, A. Voller et al., eds., University Park, 1981, Zola, Monoclonal Antibodies: A Manual of Techniques, pp. 147-158 (CRC Press, Inc. 1987).

(1) Sandwich assays involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus forming an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

In the sandwich assay, the immobilized antibody of the present invention is reacted with a test fluid (primary reaction), then with a labeled form of antibody of the present invention (secondary reaction), and the activity of the labeling agent on the immobilizing carrier is measured, whereby the Notch1 level in the test fluid can be quantified. The primary and secondary reactions may be performed simultaneously or with some time intervals. The methods of labeling and immobilization can be performed by modifications of those methods described above. In the immunoassay by the sandwich assay, the antibody used for immobilized or labeled antibody is not necessarily from one species, but a mixture of two or more species of antibodies may be used to increase the measurement sensitivity, etc. In the method of assaying Notch1 by the sandwich assay, for example, when the antibodies used in the primary reaction recognize the partial peptides at the C-terminal region of Notch1, the antibodies used in the secondary reaction are preferably those recognizing partial peptides other than the C-terminal region (i.e., the N-terminal region). When the antibodies used for the primary reaction recognize partial peptides at the N-terminal region of Notch1, the antibodies used in the secondary reaction, antibodies recognizing partial peptides other than the N-terminal region (i.e., the C-terminal region) are preferably employed.

Other types of “sandwich” assays, which can also be useful for detecting Notch1, are the so-called “simultaneous” and “reverse” assays. A simultaneous assay involves a single incubation step wherein the antibody bound to the solid support and labeled antibody are both added to the sample being tested at the same time. After the incubation is completed, the solid support is washed to remove the residue of fluid sample and uncomplexed labeled antibody. The presence of labeled antibody associated with the solid support is then determined as it would be in a conventional “forward” sandwich assay.

In the “reverse” assay, stepwise addition first of a solution of labeled antibody to the fluid sample followed by the addition of unlabeled antibody bound to a solid support after a suitable incubation period, is utilized. After a second incubation, the solid phase is washed in conventional fashion to free it of the residue of the sample being tested and the solution of unreacted labeled antibody. The determination of labeled antibody associated with a solid support is then determined as in the “simultaneous” and “forward” assays. In one embodiment, a combination of antibodies of the present invention specific for separate epitopes can be used to construct a sensitive three-site immunoradiometric assay.

This type of assays may also be used to quantify Notch1 expression in whatever “sample” it may present itself. Thus, in certain aspects, the sandwich assay includes:

(i) a method for quantifying expression levels of Notch1 in a test fluid, comprising reacting the antibody specifically reacting with a partial peptide at the N-terminal region of the Notch1 immobilized on a carrier, a labeled form of the antibody specifically reacting with a partial peptide at the C-terminal region and the test fluid, and measuring the activity of the label; or

(ii) a method for quantifying Notch1 expression in a test fluid, comprising reacting the antibody specifically reacting with a partial peptide at the C-terminal region of the Notch1 immobilized onto a carrier, the antibody specifically reacting with a partial peptide at the N-terminal region of a labeled form of the Notch1 and the test fluid, and measuring the activity of the label; etc.

### (2) Competitive Assay

Competitive binding assays rely on the ability of a labeled standard to compete with the test sample analyte for binding with a limited amount of antibody. The amount of Notch1 protein in the test sample is inversely proportional to the amount of standard that becomes bound to the antibodies. To facilitate determining the amount of standard that becomes bound, the antibodies generally are insolubilized before or after the competition, so that the standard and analyte that are bound to the antibodies may conveniently be separated from the standard and analyte which remain unbound.

For quantifying the level of Notch1 expression, one skilled in the art may combine and/or competitively react antibodies of the invention or fragments thereof, a test fluid and a labeled form of Notch1, measure a ratio of the labeled Notch1 bound to the antibodies or fragments thereof b to thereby quantify the Notch1 in the test fluid.

### (3) Immunometric Assay

In the immunometric assay, an antigen in a test fluid and a solid phase antigen are competitively reacted with a given amount of a labeled form of the antibody of the present invention followed by separating the solid phase from the liquid phase; or an antigen in a test fluid and an excess amount of labeled form of the antibody of the present invention are reacted, then a solid phase antigen is added to bind an unreacted labeled form of the antibody of the present invention to the solid phase and the solid phase is then separated from the liquid phase. Thereafter, the labeled amount of any of the phases is measured to determine the antigen level in the test fluid.

Typical, and preferred, immunometric assays include “forward” assays in which the antibody bound to the solid phase is first contacted with the sample being tested to extract the Notch1 from the sample by formation of a binary solid phase antibody-Notch1 complex. After a suitable incubation period, the solid support is washed to remove the residue of the fluid sample, including unreacted Notch1, if any, and then contacted with the solution containing a known quantity of labeled antibody (which functions as a “reporter molecule”). After a second incubation period to permit the labeled antibody to complex with the Notch1 bound to the solid support through the unlabeled antibody, the solid support is washed a second time to remove the unreacted labeled antibody. This type of forward sandwich assay can be a simple “yes/no” assay to determine whether Notch1 is present or can be made quantitative by comparing the measure of labeled antibody with that obtained for a standard sample containing known quantities of Notch1. Such “two-site” or “sandwich” assays are described by Wide (Radioimmune Assay Method, Kirkham, ed., Livingstone, Edinburgh, 1970, pp. 199 206).

### (4) Nephrometry

In the nephrometry, the amount of insoluble sediment, which is produced as a result of the antigen-antibody reaction in a gel or in a solution, is measured. Even when the amount of an antigen in a test fluid is small and only a small amount of the sediment is obtained, a laser nephrometry utilizing laser scattering can be suitably used.

Examples of labeling agents, which may be used in the above referenced assay methods (1) to (4) using labeling agents, include radioisotopes (e.g., ¹²⁵I, ¹³¹I, ³H, ¹⁴C, ³²P, ³³P, ³⁵S, etc., fluorescent substances, e.g., cyanine fluorescent dyes (e.g., Cy2, Cy3, Cy5, Cy5.5, Cy7), fluorescamine, fluorescein isothiocyanate, etc., enzymes (e.g., β-galactosidase, β-glucosidase, alkaline phosphatase, peroxidase, malate dehydrogenase, etc.), luminescent substances (e.g., luminol, a luminol derivative, luciferin, lucigenin, etc.), biotin, lanthanides, etc. In addition, a biotin-avidin system may be used as well for binding an antibody to a labeling agent.

In the immobilization of antigens or antibodies, physical adsorption may be used. Alternatively, chemical binding that is conventionally used for immobilization of proteins, enzymes, etc. may be used as well. Examples of the carrier include insoluble polysaccharides such as agarose, dextran, cellulose, etc.; synthetic resins such as polystyrene, polyacrylamide, silicone, etc.; or glass; and the like.

In certain embodiments, the antibodies of the present invention are tested for their antigen binding activity. The antigen binding assays that are known in the art and can be used herein include without limitation any direct or competitive binding assays using techniques such as western blots, radioimmunoassays, ELISA (enzyme linked immunosorbent assay), “sandwich” immunoassays, immunoprecipitation assays, fluorescent immunoassays, and protein A immunoassays. Illustrative antigen binding assay are provided herein.

In some embodiments, the binding affinity of anti Notch1 antibodies is determined. Antibodies of the invention preferably have a binding affinity (K_{D}) to Notch1 of at least about 1×10⁻⁷ M, more preferably at least about 1.times.10⁻⁸ M, more preferably at least about 1.times.10⁻⁹ M, and most preferably at least about 1.times.10⁻¹⁰ M. Preferred antibody-producing cells of the invention produce substantially only antibodies having a binding affinity to Notch1 of at least about 1.times.10⁻⁷ M, more preferably at least about 1.times.10⁻⁸ M, more preferably at least about 1.times.10⁻⁹ M, and most preferably at least about 1.times.10⁻¹⁰ M. Preferred compositions of the invention comprise substantially only antibodies having a binding affinity to Notch1 of at least about 1.times.10⁻⁷ M, more preferably at least about 1.times.10⁻⁸ M, more preferably at least about 1.times.10⁻⁹ M, and most preferably at least about 1.times.10⁻¹⁰ M.

In another aspect of the invention, the antibodies of the invention bind to Notch1 with substantially the same K_{D} as an antibody that comprises one of the amino acid sequences selected from SEQ ID NOs: 3-22; 23-42; 43-45; 46-48; 49-76 and/or 77-104. In another embodiment, the antibody binds to Notch1 with substantially the same K_{D} as an antibody that comprises one or more CDRs selected from the group consisting of SEQ ID NOS: 207-221 (heavy chain) or SEQ ID NOs:222-253 (light chain).

Anti-Notch1 antibodies according to the invention or identified using the methods disclosed herein have a low dissociation rate. In one embodiment, the anti-Notch1 antibody has a K_{off} of 1×0.10⁻⁴ or lower, preferably a K_{off} that is 5×10⁻⁵ or lower. In another embodiment, the antibodies of to invention or those identified or produced using the methods of the invention bind to Notch1 with substantially the same K_{off} as an antibody that comprises one or more CDRs disclosed herein. Illustrative assays for affinity analysis are described herein.

### Affinity Analysis for Epitope(s)

Affinity can be either absolute or relative. By absolute affinity, it is meant that the assay for affinity gives defined numerical determinations of the affinity of one compound for another. Comparison of the affinity of the complex being tested to that of a reference compound whose binding affinity is known allows for the determination of relative binding affinity of the test ligand.

Whether absolute or relative, affinity of one molecule for another can be measured by any method known in the art. By way of non-limiting example, such methods include competition assays, surface plasmon resonance, half-maximal binding assays, competition assays, Scatchard analysis, direct force techniques (Wong et al., Direct force measurements of the streptavidin-biotin interaction, Biomol. Eng. 16:45-55, 1999), and mass spectrometry (Downard, Contributions of mass spectrometry to structural immunology, J. Mass Spectrom. 35:493-503, 2000).

The binding affinity and dissociation rate of an antibody to Notch1 may be determined by any method known in the art. For example, the binding affinity can be measured by competitive ELISAs, RIAs or surface plasmon resonance, such as BIAcore. The dissociation rate can also be measured by surface plasmon resonance. Alternatively, the binding affinity and dissociation rate is measured by surface plasmon resonance. More, the binding affinity and dissociation rate is measured using a BIAcore. See below for a brief description, it being understood the invention is not limited to the specific assays detailed herein.

1. Absolute Affinity

As regards absolute affinity, “low affinity” refers to binding wherein the association constant (Kₐ) between two molecules is about 10⁵ M⁻¹ to 10⁷ M⁻¹. “Moderate affinity” refers to binding wherein the association constant (Kₐ) between two molecules is at least about 10⁷ M⁻¹ to 10⁸ M⁻¹. “High affinity” refers to a binding wherein the association constant between the two molecules is at least about 10⁸ M⁻¹ to about 10¹⁴ M⁻¹, and preferably about 10⁹ M⁻¹ to about 10¹⁴ M⁻¹, more preferably about 10¹⁰ M⁻¹ to about 10¹⁴ M⁻¹, and most preferably greater than about 10¹⁴ M⁻¹.

The dissociation constant, K_{d}, is an equilibrium constant for the dissociation of one species into two, e.g., the dissociation of a complex of two or more molecules into its components, for example, dissociation of a substrate from an enzyme. Exemplary K_{d} values for compositions of the present invention are from about 10⁻⁷ M (100 nM) to about 10⁻¹² M (0.001 nM). The stability constant is an equilibrium constant that expresses the propensity of a species to form from its component parts. The larger the stability constant, the more stable is the species. The stability constant (formation constant) is the reciprocal of the instability constant (dissociation constant).

The affinity of an invention antibody for a target epitope, or the affinity of a bi-specific antibody for a carrier epitope, is driven by non-covalent interactions. There are four main non-covalent attractive forces between molecules: (i) electrostatic forces, which occur between oppositely charged molecules such as amino groups and carboxylic groups; (ii) hydrogen bonds, which are formed when hydrogen atoms are shared between electronegative atoms such as nitrogen and oxygen; (iii) Van der Waals forces, which are generated between electron clouds around molecules oppositely polarized by neighboring atoms; and (iv) hydrophobic interactions, which are formed when water is excluded from the interface allowing hydrophobic molecules to interact in a waterless environment.

Non-covalent interactions can, but rarely do, have the strength of a covalent linkage (i.e., a chemical bond). In some instances, the affinity of the invention antibody for a target epitope, although driven by non-covalent interactions, is so high as to approach the strength of a covalent bond. This provides for invention antibodies that are very stable relative to other Notch receptor antibodies of the invention.

Preferably, the affinity of an invention antibody for its cognate target epitope, is a K_{D} of about 100 nM to about 0.01 nM; more preferably, greater than about 100 nM, or greater than about 10 nM; most preferably, greater than about 1 nM, or greater than about 0.1 nM. Typical K_{D} for target epitopes are from about 0.1 nM to 100 nM, preferably from about 0.1 nM to 10 nM, more preferably from about 0.5 nM to 5 nM, or about 1 nM.

In the invention, when multiple copies of a carrier epitope are present on the antibody, the affinity of an antibody for its cognate carrier epitope may be greater than the affinity of an antibody for a free carrier epitope or for a monovalent antibody comprising the carrier epitope. Additionally or alternatively, a multivalent targetable construct having x carrier epitopes has a greater affinity for its target epitope than would x number of constructs. Put another way, the compositions of the invention also provides for synergistic, rather than merely additive, binding effects.

2. Surface Plasmon Resonance

Binding parameters such as K_{D} may be measured using surface plasmon resonance on a chip, for example, with a BIAcore® chip coated with immobilized binding components. Surface plasmon resonance is used to characterize the microscopic association and dissociation constants of reaction between an antibody or antibody fragment and its ligand. Such methods are generally described in the following references which are incorporated herein by reference. (Vely et al., BIAcore analysis to test phosphopeptide-SH2 domain interactions, Meth. Mol. Biol. 121:313-21, 2000; Liparoto et al., Biosensor analysis of the interleukin-2 receptor complex, J. Mol. Recog. 12:316-21, 1999; Lipschultz et al., Experimental design for analysis of complex kinetics using surface plasmon resonance, Methods 20:310-8, 2000; Malmqvist., BIACORE: an affinity biosensor system for characterization of biomolecular interactions, Biochem. Soc. Transactions 27:33540, 1999; Alfthan, Surface plasmon resonance biosensors as a tool in antibody engineering, Biosensors & Bioelectronics 13:653-63, 1998; Fivash et al., BIAcore for macromolecular interaction, Curr. Opin. Biotech. 9:97-101, 1998; Price et al., Summary report on the ISOBM TD-4 Workshop: analysis of 56 monoclonal antibodies against the MUC1 mucin, Tumour Biol. 19 Suppl 1:1-20, 1998; Malmqvist et al., Biomolecular interaction analysis: affinity biosensor technologies for functional analysis of proteins, Curr. Opin. Chem. Biol. 1:378-83, 1997; O'Shannessy et al., Interpretation of deviations from pseudo-first-order kinetic behavior in the characterization of ligand binding by biosensor technology, Anal. Biochem. 236:275-83, 1996; Malmborg et al., BIAcore as a tool in antibody engineering, J. Immunol. Meth. 183:7-13, 1995; Van Regenmortel, Use of biosensors to characterize recombinant proteins, Dev. Biol. Standardization 83:143-51, 1994; O'Shannessy, Determination of kinetic rate and equilibrium binding constants for macromolecular interactions: a critique of the surface plasmon resonance literature, Curr. Opin. Biotechnol. 5:65-71, 1994). Models using BIAcore to examine the binding of fixed ligands to multivalent compounds have been described (Muller et al., Model and simulation of multivalent binding to fixed ligands, Anal. Biochem. 261:149-158, 1998).

BIAcore® uses the optical properties of surface plasmon resonance (SPR) to detect alterations in protein concentration bound within to a dextran matrix lying on the surface of a gold/glass sensor chip interface, a dextran biosensor matrix. In brief, proteins are covalently bound to the dextran matrix at a known concentration and a ligand for the protein (e.g., antibody) is injected through the dextran matrix. Near infrared light, directed onto the opposite side of the sensor chip surface is reflected and also induces an evanescent wave in the gold film, which in turn, causes an intensity dip in the reflected light at a particular angle known as the resonance angle. If the refractive index of the sensor chip surface is altered (e.g., by ligand binding to the bound protein) a shift occurs in the resonance angle. This angle shift can be measured and is expressed as resonance units (RUs) such that 1000 RUs is equivalent to a change in surface protein concentration of 1 ng/mm 2. These changes are displayed with respect to time along the y-axis of a sensorgram, which depicts the association and dissociation of any biological reaction.

Additional details may be found in Jonsson et al., Introducing a biosensor based technology for real-time biospecific interaction analysis, Ann. Biol. Clin. 51:19-26, 1993; Jonsson et al., Real-time biospecific interaction analysis using surface plasmon resonance and a sensor chip technology, Biotechniques 11:620-627, 1991; Johnsson et al., Comparison of methods for immobilization to carboxymethyl dextran sensor surfaces by analysis of the specific activity of monoclonal antibodies, J. Mol. Recog. 8:125-131, 1995; and Johnsson, Immobilization of proteins to a carboxymethyldextran-modified gold surface for biospecific interaction analysis in surface plasmon resonance sensors, Anal. Biochem. 198:268-277, 1991; Karlsson et al., Kinetic analysis of monoclonal antibody-antigen interactions with a new biosensor based analytical system, J. Immunol. Meth. 145:229, 1991; Weinberger et al., Recent trends in protein biochip technology, Pharmacogenomics 1:395-416, 2000; Lipschultz et al., Experimental design for analysis of complex kinetics using surface plasmon resonance, Methods 20:310-8, 2000.

3. Relative Affinity

Affinity may also be defined in relative terms, e.g., by IC₅₀. In the context of affinity, the IC₅₀ of a compound is the concentration of that compound at which 50% of a reference ligand is displaced from a target epitope in vitro or targeted tissue in vivo. Typically, IC₅₀ is determined by competitive ELISA. In still another embodiment, the invention provides anti-Notch1 monoclonal antibodies that compete with a conventional anti-Notch antibody for binding to Notch receptor protein. Such competitor antibodies include antibodies that recognize a Notch1 epitope that is the same as or overlaps with the Notch1 epitope recognized by any one of a conventional antibody. Such competitor antibodies can be obtained by assay well known to one skilled in the art. For example, they can b obtained by screening anti-Notch1 hybridoma supernatants for binding to immobilized Notch1 in competition with labeled 26.6, 26.14, 26.20, 26.34, and/or 26.82 antibodies. Alternatively, they can be used in a binding assay A hybridoma supernatant containing competitor antibody will reduce the amount of bound, labeled antibody detected in the subject competition binding mixture as compared to the amount of bound, labeled antibody detected in a control binding mixture containing irrelevant (or no) antibody. Any of the competition binding assays described herein are suitable for use in the foregoing procedure.

Anti-Notch1 antibodies of the invention possessing the unique properties described herein can be obtained by screening anti-Notch1 hybridoma clones for the desired properties by any convenient method. For example, if an anti-Notch1 monoclonal antibody that blocks or does not block the binding of Notch receptors to its binding partner e.g., a Notch ligand is desired, the candidate antibody can be tested in a binding competition assay, such as a competitive binding ELISA, wherein plate wells are coated with the binding partner, and a solution of antibody in an excess of the Notch receptor of interest is layered onto the coated plates, and bound antibody is detected enzymatically, e.g. contacting the bound antibody with HRP-conjugated anti-Ig antibody or biotinylated anti-Ig antibody and developing the HRP color reaction., e.g. by developing plates with streptavidin-HRP and/or hydrogen peroxide and detecting the HRP color reaction by spectrophotometry at 490 nm with an ELISA plate reader.

In one embodiment, the present invention contemplates an altered antibody that possesses some but not all effector functions, which make it a desired candidate for many applications in which the half life of the antibody in vivo is important yet certain effector functions (such as complement and ADCC) are unnecessary or deleterious. In certain embodiments, the Fc activities of the produced immunoglobulin are measured to ensure that only the desired properties are maintained. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu Rev. Immunol 9:457-92 (1991). An example of an in vitro assay to assess ADCC activity of a molecule of interest is described in U.S. Pat. No. 5,500,362 or 5,821,337. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Natl. Acad. Sci. A 95:652-656 (1998). C1q binding assays may also be carried out to confirm that the antibody is unable to bind C1q and hence lacks CDC activity. To assess complement activation, a CDC assay, e.g. as described in Gazzano-Santoro et al., J. Immunol. Methods 202:163 (1996), may be performed. FcRn binding and in vivo clearance/half life determinations can also be performed using methods known in the art, e.g. those described in the Examples section.

### Identification of Notch1 Epitopes Recognized by Anti-Notch1 Antibody

One may determine whether an anti-Notch1 antibody derived from the antibodies of the invention or produced in accordance with the methods described above binds to the same antigen as another antibody, e.g., conventional antibody using a variety of methods known in the art. For instance, one may determine whether a test anti-Notch1 antibody binds to the same antigen by using an anti-Notch1 antibody to capture an antigen that is known to bind to the anti-Notch1 antibody, eluting the antigen from the antibody, and then determining whether the test antibody will bind to the eluted antigen.

One may determine whether a test antibody binds to the same epitope as an anti-Notch1 antibody by binding the anti-Notch1 antibody to Notch1 receptor protein under saturating conditions, and then measuring the ability of the test antibody to bind to Notch1. If the test antibody, e.g., anti-Notch1 antibodies derived from the invention antibodies or in accordance with the methods of the invention is able to bind to the Notch1 receptor protein at the same time as the reference anti-Notch1 antibody, then the test antibody binds to a different epitope as the anti-Notch1 antibody. However, if the test antibody is not able to bind to Notch1 receptor protein at the same time, then the test antibody binds to the same epitope as the human anti-Notch1 antibody. This experiment may be performed using ELISA, RIA or surface plasmon resonance. In certain embodiments, the experiment is performed using surface plasmon resonance, supra. In another embodiment, BIAcore is used, see supra. One may also determine whether an anti-Notch1 antibody cross-competes with a reference anti-Notch1 antibody. For example, one may determine whether a test anti-Notch1 antibody cross-competes with another by using the same method that is used to measure whether the anti-Notch1 antibody is able to bind to the same epitope as another anti-Notch1 antibody.

The diagnostic method may also be used to determine whether a tumor is potentially cancerous, if it expresses high levels of Notch1, or benign, if it expresses low levels of Notch1. Thus, for example, biological samples obtained from patients suspected of exhibiting an oncogenic disorder mediated by Notch1 may be assayed for the presence of Notch1 expressing cells.

As noted, the anti-Notch1 antibodies of the invention may be used to determine the levels of Notch1 receptor protein in a tissue or in cells derived from the tissue. In one embodiment, the tissue is a diseased tissue. In a more preferred embodiment, the tissue is a tumor or a biopsy thereof. In a preferred embodiment of the method, a tissue or a biopsy thereof is excised from a patient. The tissue or biopsy is then used in an immunoassay to determine, e.g., Notch1 levels, cell surface levels of Notch1, levels of tyrosine phosphorylation of Notch1, or localization of Notch1 by the methods discussed herein. The method can be used to determine tumors that express Notch1.

In a related embodiment, the present invention provides methods for diagnosing cancers by assaying for changes in the level of Notch1 in cells, tissues or body fluids compared with the levels in cells, tissues, or body fluids, preferably of the same type in a control sample. A change, especially an increase, in levels of Notch1 in the patient versus the control is associated with the presence of cancer. Typically, for a quantitative diagnostic assay, a positive result indicating that the patient being tested has cancer is one in which levels of Notch1 in or on cells, tissues or body fluid are at least two times higher, and preferably three to five times higher, or greater, than the levels of the antigens in or on the same cells, tissues, or body fluid of the control. Normal controls include a human without cancer and/or non-cancerous samples from the patient.

The in vitro diagnostic methods may include any method known to one skilled in the art including immunohistological or immunohistochemical detection of tumor cells (e.g., on human tissue, or on cells dissociated from excised tumor specimens), or serological detection of tumor associated antigens (e.g., in blood samples or other biological fluids). Immunohistochemical techniques involve staining a biological specimen, such as a tissue specimen, with one or more of the antibodies of the invention and then detecting the presence on the specimen of antibody-antigen complexes comprising antibodies bound to the cognate antigen. The formation of such antibody-antigen complexes with the specimen indicates the presence of cancer in the tissue.

Detection of the antibody on the specimen can be accomplished using techniques known in the art such as immunoenzymatic techniques, e.g., immunoperoxidase staining technique, or the avidin-biotin technique, or immunofluorescence techniques (see, e.g., Ciocca et al., 1986, “Immunohistochemical Techniques Using Monoclonal Antibodies”, Meth. Enzymol., 121:562 79 and Introduction to Immunology, Ed. Kimball, (2^{nd} Ed), Macmillan Publishing Company, 1986, pp. 113 117). Those skilled in the art can determine operative and optimal assay conditions by routine experimentation.

In another embodiment, the present invention assists in the diagnosis of cancers and tumors by the identification and measurement of the Notch1 receptor protein levels in biological samples. If Notch1 receptor protein is normally present, and the development of the oncogenic disorder is caused by an abnormal quantity of the cell surface receptor (Notch1), e.g., expression relative to normal, the assay should compare Notch1 levels in the biological sample to the range expected in normal, non-oncogenic tissue of the same cell type. Thus, a statistically significant increase in the amount of Notch1 bearing cells or Notch1 expression level in the subject relative to the control subject or subject's baseline, can be a factor that may lead to a diagnosis of an oncogenic disorder that is progressing or at risk for such a disorder. Likewise, the presence of high levels of Notch1 indicative of cancers likely to metastasize can also be detected. For those cancers that express the antigen recognized by the antibodies of the invention, e.g., Notch1, the ability to detect the antigen provides early diagnosis, thereby affording the opportunity for early treatment. Early detection is especially important for cancers difficult to diagnose in their early stages.

Moreover, the level of antigen detected and measured in a body fluid sample such as for example diseased tissue provides a means for monitoring the course of therapy for the cancer or tumor, including, but not limited to, surgery, chemotherapy, radiation therapy, the therapeutic methods of the present invention, and combinations thereof. By correlating the level of the antigen in the tissue sample with the severity of disease, the level of such antigen can be used to indicate successful removal of the primary tumor, cancer, and/or metastases, for example, as well as to indicate and/or monitor the effectiveness of other therapies over time. For example, a decrease in the level of the cancer or tumor-specific antigen over time indicates a reduced tumor burden in the patient. By contrast, no change, or an increase, in the level of antigen over time indicates ineffectiveness of therapy, or the continued growth of the tumor or cancer.

A typical in vitro immunoassay for detecting Notch1 comprises incubating a biological sample in the presence of a detectably labeled anti-Notch1 antibody or antigen binding fragment of the present invention capable of selectively binding to Notch1, and detecting the labeled fragment or antibody which is bound in a sample. The antibody is bound to a label effective to permit detection of the cells or portions (e.g., Notch1 or fragments thereof liberated from hyperplastic, dysplastic and/or cancerous cells) thereof upon binding of the antibody to the cells or portions thereof. The presence of any cells or portions thereof in the biological sample is detected by detection of the label.

The biological sample may be brought into contact with, and immobilized onto, a solid phase support or carrier, such as nitrocellulose, or other solid support or matrix, which is capable of immobilizing cells, cell particles, membranes, or soluble proteins. The support may then be washed with suitable buffers, followed by treatment with the detectably-labeled anti-Notch1 antibody. The solid phase support may then be washed with buffer a second time to remove unbound antibody. The amount of bound label on the solid support may then be detected by conventional means. Accordingly, in another embodiment of the present invention, compositions are provided comprising the monoclonal antibodies, or binding fragments thereof, bound to a solid phase support, such as described herein.

In vitro assays in accordance with the present invention also include the use of isolated membranes from cells expressing a recombinant Notch1, soluble fragments comprising the ligand binding segments of Notch1, or fragments attached to solid phase substrates. These assays allow for the diagnostic determination of the effects of either binding segment mutations and modifications, or ligand mutations and modifications, e.g., ligand analogues.

In certain embodiments the monoclonal antibodies and binding fragments thereof of the present invention may be used in in vitro assays designed to screen compounds for binding affinity to Notch1. See Fodor et al. Science 251: 767-773 (1991), incorporated herein by reference. In accordance with this objective, the invention contemplates a competitive drug screening assay, where the monoclonal antibodies or fragments thereof of the invention compete with a test compound for binding to Notch1. In this manner the monoclonal antibodies and fragments thereof are used to detect the presence of any polypeptide which shares one or more binding sites of the Notch1 and can be used to occupy binding sites on the receptor which might otherwise be occupied by the antibody.

In certain embodiments, the anti-Notch1 antibodies of the invention may be used to determine or quantify the amount of Notch1 on the cell surface after treatment of the cells with various compounds. This method can be used to test compounds that may be used to activate or inhibit Notch1. In this method, one sample of cells is treated with a test compound for a period of time while another sample is left untreated. If the total level of Notch1 is to be measured, the cells are lysed and the total Notch1 level is measured using one of the immunoassays described herein.

A preferred immunoassay for measuring total Notch1 receptor protein levels is an ELISA or Western blot. If only the cell surface level of Notch1 is to be measured, the cells are not lysed, and the cell surface levels of Notch1 are measured using any one or more of the assays known to the skilled artisan, e.g., one of the immunoassays described herein. A preferred immunoassay for determining cell surface levels of Notch1 includes the steps of labeling the cell surface proteins with a detectable label, such as biotin or ¹²⁵I, immunoprecipitating the Notch1 with an anti-Notch1 antibody and then detecting the labeled Notch1. Another preferred immunoassay for determining the localization of Notch1, e.g., cell surface levels, is by using immunohistochemistry.

As well, provided herein is a method to determine whether a conventional anti-Notch1 antibody decreases Notch1 expression on a target tumor tissue or cell. The term “conventional Notch1 antagonist” “conventional treatment with a Notch1 moiety” is used interchangeably to mean Notch1 specific monoclonal antibodies currently available that specifically target Notch1 expression and do not bind to the same epitope as the antibodies of the invention.

A further aspect of the invention is an assessment of the susceptibility that an individual has for developing cancer mediated by Notch1. The method comprises the steps of measuring the level of expression of Notch1 in a cell or tissue of interest, incubating the cell or tissue with an anti-Notch1 antibody or antigen-binding portion thereof, then re-measuring the level of Notch1 expression with an anti-Notch1 antibody or antigen binding fragment of the invention in the cell or tissue. Alternatively, ICD expression levels may be measured in the above example. A diagnosis that levels of Notch1 are low could be used for predicting that the patient is responding to treatment with the conventional anti-Notch1 antibody regiment. On the contrary, no change in the level of Notch1 or an increase in expression of Notch1 after treatment with a conventional anti-Notch1 antibody indicate that the patient is either unresponsive to the current treatment protocol or unlikely to respond to further treatment with the conventional anti-Notch1 antibody, thereby allowing for earlier intervention. The anti-Notch1 antibodies of the invention may be used in the above diagnostic assays either simultaneously with administration of the conventional Notch1 antibody or after treatment with the conventional anti-Notch1. Preferably, the conventional Notch1 antibody does not compete with the anti-Notch1 antibody of the invention for binding Notch1 protein. The above assays can be performed iteratively over a period of time to assess the therapeutic efficacy of a conventional anti-Notch1 antibody based therapeutic protocol. In this way, the anti-Notch1 antibody of the invention can be used as a “negative biomarker” allowing it to be used to assess the treatment and therapeutic protocol of a conventional anti-Notch1 antibody based therapy.

### Vectors, Host Cells and Recombinant Methods

The invention also includes nucleic acids encoding the heavy chain and/or light chain of the anti-Notch1 antibodies of the invention. Nucleic acids of the invention also include fragments of the nucleic acids of the invention. A “fragment” refers to a nucleic acid sequence that is preferably of sufficient length to encode a functionally active fragment of the invention antibodies, e.g., light or heavy chain. A “fragment” can also mean the whole coding sequence of a gene and may include 5′ and 3′ untranslated regions.

Constructs of any one or more polynucleotides having sequences as set forth herein can be generated synthetically. Alternatively, single-step assembly of a gene and entire plasmid from large numbers of oligodeoxyribonucleotides is described by, e.g., Stemmer et al., Gene (Amsterdam) (1995) 164(1):49-53. In this method, assembly PCR (the synthesis of long DNA sequences from large numbers of oligodeoxyribonucleotides) is derived from DNA shuffling (Stemmer, Nature (1994) 370:389-391).

Appropriate polynucleotide constructs are purified using standard recombinant DNA techniques as described in, for example, Sambrook et al., Molecular Cloning: A Laboratory Manual, 2nd Ed., (1989) Cold Spring Harbor Press, Cold Spring Harbor, N.Y. The gene product encoded by a polynucleotide of the invention is expressed in any expression system, including, for example, bacterial, yeast, insect, amphibian and mammalian systems. Vectors, host cells and methods for obtaining expression in same are well known in the art. Suitable vectors and host cells are described in U.S. Pat. No. No. 5,654,173.

Polynucleotide molecules comprising a polynucleotide sequence provided herein are generally propagated by placing the molecule in a vector. Viral and non-viral vectors are used, including plasmids. The choice of plasmid will depend on the type of cell in which propagation is desired and the purpose of propagation. Certain vectors are useful for amplifying and making large amounts of the desired DNA sequence. Other vectors are suitable for expression in cells in culture. Still other vectors are suitable for transfer and expression in cells in a whole animal or person. The choice of appropriate vector is well within the skill of the art. Many such vectors are available commercially. Methods for preparation of vectors comprising a desired sequence are well known in the art.

The polynucleotides set forth in one of SEQ ID NOs: 105-134 or 125-144 or 145-147 or 148-150 or their corresponding full-length polynucleotides are linked to regulatory sequences as appropriate to obtain the desired expression properties. These can include promoters (attached either at the 5′ end of the sense strand or at the 3′ end of the antisense strand), enhancers, terminators, operators, repressors, and inducers. The promoters can be regulated or constitutive. In some situations it may be desirable to use conditionally active promoters, such as tissue-specific or developmental stage-specific promoters. These are linked to the desired nucleotide sequence using the techniques described above for linkage to vectors. Any techniques known in the art can be used.

When any appropriate host cells or organisms are used to replicate and/or express the polynucleotides or nucleic acids of the invention, the resulting replicated nucleic acid, RNA, expressed protein or polypeptide, is within the scope of the invention as a product of the host cell or organism. The product is recovered by any appropriate means known in the art.

Expression of a target gene, e.g., corresponding to any one or more of the nucleic acid molecules set forth herein can be regulated in the cell to which the gene is native. For example, an endogenous gene of a cell can be regulated by an exogenous regulatory sequence as disclosed in U.S. Pat. No. 5,641,670.

The encoded antibody heavy chain preferably comprises an amino acid sequence selected from the group consisting of SEQ ID NOs: 3-22 and 43-45. The encoded antibody light chain preferably comprises an amino acid sequence of SEQ ID NO: 23-42 and 46-48.

In some embodiments, the invention provides nucleic acids encoding both a heavy chain and a light chain of an antibody of the invention. For example, a nucleic acid of the invention may comprise a nucleic acid sequence (SEQ ID NOs: 105-124) encoding an amino acid sequence of SEQ ID NOs: 3-22 and a nucleic acid sequence (SEQ ID Nos: 125-144) encoding an amino acid sequence of SEQ ID NOs: 23-42.

Nucleic acids of the invention include nucleic acids having at least 80%, more preferably at least about 90%, more preferably at least about 95%, and most preferably at least about 98% homology to nucleic acids of the invention. The terms “percent similarity”, “percent identity” and “percent homology” when referring to a particular sequence, are used as set forth in the University of Wisconsin GCG software program. Nucleic acids of the invention also include complementary nucleic acids. In some instances, the sequences will be fully complementary (no mismatches) when aligned. In other instances, there may be up to about a 20% mismatch in the sequences.

The invention also provides a nucleic acid molecule encoding the variable region of the light chain (V_{L}) as described herein as well as an amino acid sequence that is at least 70%, 75%, 80%, 85%, 90%, 95%, 96%, 97%, 98% or 99% identical to one of the amino acid sequences encoding a V_{L} as described herein, particularly to a V_{L} that comprises an amino acid sequence of one of SEQ ID NOs: 23-42. The invention also provides a nucleic acid sequence that is at least 70%, 75%, 80%, 85%, 90%, 95%, 96%, 97%, 98% or 99% identical to a nucleic acid sequence of one of SEQ ID NOs: 125-144. In another embodiment, the nucleic acid molecule encoding a V_{L} is one that hybridizes under highly stringent conditions to a nucleic acid sequence encoding a V_{L} as described above.

The invention also provides a nucleic acid molecule encoding the variable region of the heavy chain (V_{H}) as described herein as well as an amino acid sequence that is at least 70%, 75%, 80%, 85%, 90%, 95%, 96%, 97%, 98% or 99% identical to one of the amino acid sequences encoding a V_{H} as described herein, particularly to a V_{H} that comprises an amino acid sequence of one of SEQ ID NOs: 3-22. The invention also provides a nucleic acid sequence that is at least 70%, 75%, 80%, 85%, 90%, 95%, 96%, 97%, 98% or 99% identical to a nucleic acid sequence of one of SEQ ID NOs: 105-124. In another embodiment, the nucleic acid molecule encoding a V_{H} is one that hybridizes under highly stringent conditions to a nucleic acid sequence encoding a V_{H} as described above.

The term “selectively hybridize” referred to herein means to detectably and specifically bind. Polynucleotides, oligonucleotides and fragments thereof in accordance with the invention selectively hybridize to nucleic acid strands under hybridization and wash conditions that minimize appreciable amounts of detectable binding to nonspecific nucleic acids. “High stringency” or “highly stringent” conditions can be used to achieve selective hybridization conditions as known in the art and discussed herein. An example of “high stringency” or “highly stringent” conditions is a method of incubating a polynucleotide with another polynucleotide, wherein one polynucleotide may be affixed to a solid surface such as a membrane, in a hybridization buffer of 6.times. SSPE or SSC, 50% formamide, 5.times. Denhardt's reagent, 0.5% SDS, 100 μg/ml denatured, fragmented salmon sperm DNA at a hybridization temperature of 42° C. for 12 16 hours, followed by twice washing at 55° C. using a wash buffer of 1.times.SSC, 0.5% SDS. See also Sambrook et al., supra, pp. 9.50 9.55.

The nucleic acid molecule encoding either or both of the entire heavy and light chains of an anti-Notch1 antibodies or the variable regions thereof may be obtained from any source that produces an anti-Notch1 antibody. Methods of isolating mRNA encoding an antibody are well-known in the art (See, e.g., Sambrook et al.) The mRNA may be used to produce cDNA for use in the polymerase chain reaction (PCR) or cDNA cloning of antibody genes.

A nucleic acid molecule encoding the entire heavy chain of an anti-Notch1 antibody disclosed herein, e.g., SEQ ID NO: 105 may be constructed by fusing a nucleic acid molecule encoding the variable domain of a heavy chain or an antigen-binding domain thereof with a constant domain of a heavy chain. Similarly, a nucleic acid molecule encoding the light chain of the anti-Notch1 antibody of the invention, e.g., SEQ ID NO: 125 may be constructed by fusing a nucleic acid molecule encoding the variable domain of a light chain or an antigen-binding domain thereof with a constant domain of a light chain. The nucleic acid molecules encoding the V_{H} and V_{L} chain may be converted to full-length antibody genes by inserting them into expression vectors already encoding heavy chain constant and light chain constant regions, respectively, such that the V_{H} segment is operatively linked to the heavy chain constant region (C_{H}) segment(s) within the vector and the V_{L} segment is operatively linked to the light chain constant region (C_{L}) segment within the vector. Alternatively, the nucleic acid molecules encoding the V_{H} or V_{L} chains are converted into full-length antibody genes by linking, e.g., ligating, the nucleic acid molecule encoding a V_{H} chain to a nucleic acid molecule encoding a C_{H} chain using standard molecular biological techniques. The same may be achieved using nucleic acid molecules encoding V_{L} and C_{L} chains. The sequences of human heavy and light chain constant region genes are known in the art. See, e.g., Kabat et al., Sequences of Proteins of Immunological Interest, 5th Ed., NIH Publ. No. 91 3242, 1991. Nucleic acid molecules encoding the full-length heavy and/or light chains may then be expressed from a cell into which they have been introduced and the anti-Notch1 antibody isolated.

The nucleic acid molecules may be used to recombinantly express large quantities of anti-Notch1 antibodies using techniques known to one skilled in the art of recombinant biologist. Likewise, the herein described nucleic acid molecules can also be used to recombinantly produce any one of the anti-Notch1 antibody variants, mutants, fragments thereof or derivatives, including single chain antibodies, bispecific, scFv etc immunoadhesins, diabodies, mutated antibodies and antibody derivatives, as described further below.

In another embodiment, the nucleic acid molecules of the invention may be used as probes or PCR primers for specific antibody sequences. For instance, a nucleic acid molecule probe may be used in diagnostic methods or a nucleic acid molecule PCR primer may be used to amplify regions of DNA that could be used, inter alia, to isolate nucleic acid sequences for use in producing variable domains of anti-Notch1 antibodies. In a preferred embodiment, the nucleic acid molecules are oligonucleotides. In a more preferred embodiment, the oligonucleotides are from highly variable regions of the heavy and light chains of the antibody of interest. In an even more preferred embodiment, the oligonucleotides encode all or a part of one or more of the CDRs.

A “vector” is a replicon, such as a plasmid, cosmid, bacmid, phage, artificial chromosome (BAC, YAC) or virus, into which another genetic sequence or element (either DNA or RNA) may be inserted so as to bring about the replication of the attached sequence or element. A “replicon” is any genetic element, for example, a plasmid, cosmid, bacmid, phage, artificial chromosome (BAC, YAC) or virus, which is capable of replication largely under its own control. A replicon may be either RNA or DNA and may be single or double stranded. In some embodiments, the expression vector contains a constitutively active promoter segment (such as but not limited to CMV, SV40, Elongation Factor or LTR sequences) or an inducible promoter sequence such as the steroid inducible pIND vector (Invitrogen), where the expression of the nucleic acid can be regulated. The expression vector can be introduced into a cell by transfection,

In addition to the antibody chain genes, the recombinant expression vectors of the invention carry regulatory sequences that control the expression of the antibody chain genes in a host cell. It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from retroviral LTRs, cytomegalovirus (CMV) (such as the CMV promoter/enhancer), Simian Virus 40 (SV40) (such as the SV40 promoter/enhancer), adenovirus, (e.g., the adenovirus major late promoter (AdMLP)), polyoma and strong mammalian promoters such as native immunoglobulin and actin promoters. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al.

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For recombinant production of an antibody of the invention, the nucleic acid encoding it is isolated and inserted into a replicable vector for further cloning (amplification of the DNA) or for expression. DNA encoding the antibody is readily isolated and sequenced using conventional procedures (e.g., by using oligonucleotide probes that are capable of binding specifically to genes encoding the heavy and light chains of the antibody). Many vectors are available. The choice of vector depends in part on the host cell to be used. Generally, preferred host cells are of either prokaryotic or eukaryotic (generally mammalian) origin. It will be appreciated that constant regions of any isotype can be used for this purpose, including IgG, IgM, IgA, IgD, and IgE constant regions, and that such constant regions can be obtained from any human or animal species.

a. Generating Antibodies Using Prokaryotic Host Cells:
i. Vector Construction

Polynucleotide sequences encoding polypeptide components of the antibody of the invention can be obtained using standard recombinant techniques. Desired polynucleotide sequences may be isolated and sequenced from antibody producing cells such as hybridoma cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. pBR322 contains genes encoding ampicillin (Amp), kanamycin (Kn) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins. Examples of pBR322 derivatives used for expression of particular antibodies are described in detail in Carter et al., U.S. Pat. No. 5,648,237.

A convenient vector is one that encodes a functionally complete human C_{H} or C_{L} immunoglobulin sequence, with appropriate restriction sites engineered so that any V_{H} or V_{L} sequence can be easily inserted and expressed, as described above. In such vectors, splicing usually occurs between the splice donor site in the inserted J region and the splice acceptor site preceding the human C region, and also at the splice regions that occur within the human C_{H} exons. Polyadenylation and transcription termination occur at native chromosomal sites downstream of the coding regions. The recombinant expression vector can also encode a signal peptide that facilitates secretion of the antibody chain from a host cell. The antibody chain gene may be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition, phage vectors containing replicon and control sequences that are compatible with the host microorganism can be used as transforming vectors in connection with these hosts. For example, bacteriophage such as λGEM™-11 may be utilized in making a recombinant vector which can be used to transform susceptible host cells such as E. coli LE392.

The expression vector of the invention may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the light or heavy chain by removing the promoter from the source DNA via restriction enzyme digestion and inserting the isolated promoter sequence into the vector of the invention. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the PhoA promoter, the β-galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleotide sequences have been published, thereby enabling a skilled worker operably to ligate them to cistrons encoding the target light and heavy chains (Siebenlist et al. (1980) Cell 20: 269) using linkers or adaptors to supply any required restriction sites.

In one aspect of the invention, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP. In one embodiment of the invention, the signal sequences used in both cistrons of the expression system are STII signal sequences or variants thereof.

In another aspect, the production of the immunoglobulins according to the invention can occur in the cytoplasm of the host cell, and therefore does not require the presence of secretion signal sequences within each cistron. In that regard, immunoglobulin light and heavy chains are expressed, folded and assembled to form functional immunoglobulins within the cytoplasm. Certain host strains (e.g., the E. coli trxB-strains) provide cytoplasm conditions that are favorable for disulfide bond formation, thereby permitting proper folding and assembly of expressed protein subunits. Proba and Pluckthun. Gene, 159:203 (1995).

Prokaryotic host cells suitable for expressing antibodies of the invention include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In one embodiment, gram-negative cells are used. In one embodiment, E. coli cells are used as hosts for the invention. Examples of E. coli strains include strain W3110 (Bachmann, Cellular and Molecular Biology, vol. 2 (Washington, D.C.: American Society for Microbiology, 1987), pp. 1190-1219; ATCC® Deposit No. 27,325) and derivatives thereof, including strain 33D3 having genotype W3110 δfhuδ (δtonA) ptr3 lac Iq lacL8 δompTδ (nmpc-fepE) degP41 kanR (U.S. Pat. No. 5,639,635). Other strains and derivatives thereof, such as E. coli 294 (ATCC 31,446), E. coli B, E. coli λ 1776 (ATCC 31,537) and E. coli RV308 (ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well known plasmids such as pBR322, pBR325, pACYC177, or pKN410 are used to supply the replicon. Typically the host cell should secrete minimal amounts of proteolytic enzymes, and additional protease inhibitors may desirably be incorporated in the cell culture.

ii. Antibody Production

Host cells are transformed with the above-described expression vectors and cultured in conventional nutrient media modified as appropriate for inducing promoters, selecting transformants, or amplifying the genes encoding the desired sequences.

Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce any one or more of the anti-Notch antibodies of the invention are grown in media known in the art and suitable for culture of the selected host cells. Examples of suitable media include luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol.

The prokaryotic host cells are cultured at suitable temperatures. For E. coli growth, for example, the preferred temperature ranges from about 20° C. to about 39° C., more preferably from about 25° C. to about 37° C., even more preferably at about 30° C. The pH of the medium may be any pH ranging from about 5 to about 9, depending mainly on the host organism. For E. coli, the pH is preferably from about 6.8 to about 7.4, and more preferably about 7.0.

If an inducible promoter is used in the expression vector of the invention, protein expression is induced under conditions suitable for the activation of the promoter. In one aspect of the invention, PhoA promoters are used for controlling transcription of the polypeptides. Accordingly, the transformed host cells are cultured in a phosphate-limiting medium for induction. Preferably, the phosphate-limiting medium is the C.R.A.P medium (see, e.g., Simmons et al., J. Immunol. Methods (2002), 263:133-147). A variety of other inducers may be used, according to the vector construct employed, as is known in the art.

In one embodiment, the expressed polypeptides of the present invention are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

Another aspect of the invention contemplates antibody production in large quantity by a fermentation process. Various large-scale fed-batch fermentation procedures are available for production of recombinant proteins. Large-scale fermentations have at least 1000 liters of capacity, preferably about 1,000 to 100,000 liters of capacity. These fermentors use agitator impellers to distribute oxygen and nutrients, especially glucose (the preferred carbon/energy source). Small scale fermentation refers generally to fermentation in a fermentor that is no more than approximately 100 liters in volumetric capacity, and can range from about 1 liter to about 100 liters.

In a fermentation process, induction of protein expression is typically initiated after the cells have been grown under suitable conditions to a desired density, e.g., an OD550 of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction time may be used.

To improve the production yield and quality of the polypeptides of the invention, various fermentation conditions can be modified. For example, to improve the proper assembly and folding of the secreted antibody polypeptides, additional vectors overexpressing chaperone proteins, such as Dsb proteins (DsbA, DsbB, DsbC, DsbD and or DsbG) or FkpA (a peptidylprolyl cis, trans-isomerase with chaperone activity) can be used to co-transform the host prokaryotic cells. The chaperone proteins have been demonstrated to facilitate the proper folding and solubility of heterologous proteins produced in bacterial host cells. Chen et al. (1999) J Bio Chem 274:19601-19605; Georgiou et al., U.S. Pat. No. 6,083,715; Georgiou et al., U.S. Pat. No. 6,027,888; Bothmann and Pluckthun (2000) J. Biol. Chem. 275:17100-17105; Ramm and Pluckthun (2000) J. Biol. Chem. 275:17106-17113; Arie et al. (2001) Mol. Microbiol. 39:199-210.

To minimize proteolysis of expressed heterologous proteins (especially those that are proteolytically sensitive), certain host strains deficient for proteolytic enzymes can be used for the present invention. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof Some E. coli protease-deficient strains are available and described in, for example, Joly et al. (1998), supra; Georgiou et al., U.S. Pat. No. 5,264,365; Georgiou et al., U.S. Pat. No. 5,508,192; Hara et al., Microbial Drug Resistance, 2:63-72 (1996).

In one embodiment, E. coli strains deficient for proteolytic enzymes and transformed with plasmids overexpressing one or more chaperone proteins are used as host cells in the expression system of the invention.

iii. Antibody Purification

Standard protein purification methods known in the art can be employed. When using recombinant techniques, the antibody can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicofin® or Millipore Pellicon® ultrafiltration unit. A protease inhibitor such as PMSF may be included in any of the foregoing steps to inhibit proteolysis and antibiotics may be included to prevent the growth of adventitious contaminants.

The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C H3 domain, the Bakerbond ABX®resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE® chromatography on an anion or cation exchange resin (such as a polyaspartic acid column), chromatofocusing, SDS-PAGE, and ammonium sulfate precipitation are also available depending on the antibody to be recovered.

In one aspect, Protein A immobilized on a solid phase is used for immunoaffinity purification of the full length antibody products of the invention. Protein A is a 41 kD cell wall protein from Staphylococcus aureas which binds with a high affinity to the Fc region of antibodies. Lindmark et al (1983) J. Immunol. Meth. 62:1-13. The solid phase to which Protein A is immobilized is preferably a column comprising a glass or silica surface, more preferably a controlled pore glass column or a silicic acid column. In some applications, the column has been coated with a reagent, such as glycerol, in an attempt to prevent nonspecific adherence of contaminants.

As the first step of purification, the preparation derived from the cell culture as described above is applied onto the Protein A immobilized solid phase to allow specific binding of the antibody of interest to Protein A. The solid phase is then washed to remove contaminants non-specifically bound to the solid phase. Finally the antibody of interest is recovered from the solid phase by elution.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e. g., from about 0-0.25M salt).

b. Generating Antibodies Using Eukaryotic Host Cells:

The vector components generally include, but are not limited to, one or more of the following: a signal sequence, an origin of replication, one or more marker genes, an enhancer element, a promoter, and a transcription termination sequence.

### (i) Signal Sequence Component

A vector for use in a eukaryotic host cell may also contain a signal sequence or other polypeptide having a specific cleavage site at the N-terminus of the mature protein or polypeptide of interest. The heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available.

The DNA for such precursor region is ligated in reading frame to DNA encoding the antibody.

### (ii) Origin of Replication

Generally, an origin of replication component is not needed for mammalian expression vectors. For example, the SV40 origin may typically be used only because it contains the early promoter.

(iii) Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, where relevant, or (c) supply critical nutrients not available from complex media.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the antibody nucleic acid, such as DHFR, thymidine kinase, metallothionein-I and -II, preferably primate metallothionein genes, adenosine deaminase, ornithine decarboxylase, etc.

For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity (e.g., ATCC CRL-9096).

Alternatively, host cells (particularly wild-type hosts that contain endogenous DHFR) transformed or co-transformed with DNA sequences encoding an antibody, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

### (iv) Promoter Component

Expression and cloning vectors usually contain a promoter that is recognized by the host organism and is operably linked to the antibody polypeptide nucleic acid. Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Antibody polypeptide transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. Alternatively, the Rous Sarcoma Virus long terminal repeat can be used as the promoter.

### (v) Enhancer Element Component

Transcription of DNA encoding the antibody polypeptide of this invention by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the antibody polypeptide-encoding sequence, but is preferably located at a site 5′ from the promoter.

### (vi) Transcription Termination Component

Expression vectors used in eukaryotic host cells will typically also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding an antibody. One useful transcription termination component is the bovine growth hormone polyadenylation region. See WO94/11026 and the expression vector disclosed therein.

(vii) Selection and Transformation of Host Cells

Suitable host cells for cloning or expressing the DNA in the vectors herein include higher eukaryote cells described herein, including vertebrate host cells. Propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for antibody production and cultured in conventional nutrient media modified as appropriate for inducing promoters, selecting transformants, or amplifying the genes encoding the desired sequences.

(viii) Culturing the Host Cells

The Suitable host cells for producing an antibody of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

### (ix) Purification of Antibody

When using recombinant techniques, the antibody can be produced intracellularly, or directly secreted into the medium. If the antibody is produced intracellularly, as a first step, the particulate debris, either host cells or lysed fragments, are removed, for example, by centrifugation or ultrafiltration. Where the antibody is secreted into the medium, supernatants from such expression systems are generally first concentrated using a commercially available protein concentration filter, for example, an Amicon or Millipore Pellicon® ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25 M salt).

### Immunoconjugates

The invention also pertains to immunoconjugates (interchangeably termed “antibody-drug conjugates” or “ADC”)comprising at least one invention antibody conjugated to a cytotoxic agent such as a chemotherapeutic agent, a growth inhibitory agent, a toxin (e.g., an enzymatically active toxin of bacterial, fungal, plant, or animal origin, or fragments thereof), or a radioactive isotope (i.e., a radioconjugate).

The use of antibody-drug conjugates for the local delivery of cytotoxic or cytostatic agents, i.e. drugs to kill or inhibit tumor cells in the treatment of cancer (Syrigos and Epenetos (1999) Anticancer Research 19:605-614; Niculescu-Duvaz and Springer (1997) Adv. Drg Del. Rev. 26:151-172; U.S. Pat. No. 4,975,278) allows targeted delivery of the drug moiety to tumors, and intracellular accumulation therein, where systemic administration of these unconjugated drug agents may result in unacceptable levels of toxicity to normal cells as well as the tumor cells sought to be eliminated (Baldwin et al., (1986) Lancet pp. (Mar. 15, 1986):603-05; Thorpe, (1985) “Antibody Carriers Of Cytotoxic Agents In Cancer Therapy: A Review,” in Monoclonal Antibodies '84: Biological And Clinical Applications, A. Pinchera et al. (ed.s), pp. 475-506). Maximal efficacy with minimal toxicity is sought thereby. Both polyclonal antibodies and monoclonal antibodies have been reported as useful in these strategies (Rowland et al., (1986) Cancer Immunol. Immunother., 21:183-87). Drugs used in these methods include daunomycin, doxorubicin, methotrexate, and vindesine (Rowland et al., (1986) supra). Toxins used in antibody-toxin conjugates include bacterial toxins such as diphtheria toxin, plant toxins such as ricin, small molecule toxins such as geldanamycin (Mandler et al (2000) Jour. of the Nat. Cancer Inst. 92(19):1573-1581; Mandler et al (2000) Bioorganic & Med. Chem. Letters 10: 1025-1028; Mandler et al (2002) Bioconjugate Chem. 13:786-791), maytansinoids (EP 1391213; Liu et al., (1996) Proc. Natl. Acad. Sci. USA 93:8618-8623), and calicheamicin (Lode et al (1998) Cancer Res. 58:2928; Hinman et al (1993) Cancer Res. 53:3336-3342). The toxins may effect their cytotoxic and cytostatic effects by mechanisms including tubulin binding, DNA binding, or topoisomerase inhibition. Some cytotoxic drugs tend to be inactive or less active when conjugated to large antibodies or protein receptor ligands.

ZEVALIN™ (ibritumomab tiuxetan, Biogen/Idec) is an antibody-radioisotope conjugate composed of a murine IgG1 kappa monoclonal antibody directed against the CD20 antigen found on the surface of normal and malignant B lymphocytes and ¹¹¹In or ⁹⁰Y radioisotope bound by a thiourea linker-chelator (Wiseman et al (2000) Eur. Jour. Nucl. Med. 27(7):766-77; Wiseman et al (2002) Blood 99(12):4336-42; Witzig et al (2002) J. Clin. Oncol. 20(10):2453-63; Witzig et al (2002) J. Clin. Oncol. 20(15):3262-69). Although ZEVALIN has activity against B-cell non-Hodgkin's Lymphoma (NHL), administration results in severe and prolonged cytopenias in most patients. MYLOTARG™ (gemtuzumab ozogamicin, Wyeth Pharmaceuticals), an antibody drug conjugate composed of a hu CD33 antibody linked to calicheamicin, was approved in 2000 for the treatment of acute myeloid leukemia by injection (Drugs of the Future (2000) 25(7):686; U.S. Pat. Nos. 4,970,198; 5,079,233; 5,585,089; 5,606,040; 5,693,762; 5,739,116; 5,767,285; 5,773,001). Cantuzumab mertansine (Immunogen, Inc.), an antibody drug conjugate composed of the huC242 antibody linked via the disulfide linker SPP to the maytansinoid drug moiety, DM1, is advancing into Phase II trials for the treatment of cancers that express CanAg, such as colon, pancreatic, gastric, and others. MLN-2704 (Millennium Pharm., BZL Biologics, Immunogen Inc.), an antibody drug conjugate composed of the anti-prostate specific membrane antigen (PSMA) monoclonal antibody linked to the maytansinoid drug moiety, DM1, is under development for the potential treatment of prostate tumors. The auristatin peptides, auristatin E (AE) and monomethylauristatin (MMAE), synthetic analogs of dolastatin, were conjugated to chimeric monoclonal antibodies cBR96 (specific to Lewis Y on carcinomas) and cAC10 (specific to CD30 on hematological malignancies) (Doronina et al (2003) Nature Biotechnology 21(7):778-784) and are under therapeutic development.

Chemotherapeutic agents useful in the generation of immunoconjugates are described herein (eg., above). Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. See, e.g., WO 93/21232 published Oct. 28, 1993. A variety of radionuclides are available for the production of radioconjugated antibodies. Examples include ²¹²Bi, ¹³¹I, ¹³¹In, ⁹⁰Y, and ¹⁸⁶Re. Conjugates of the antibody and cytotoxic agent are made using a variety of bifunctional protein-coupling agents such as N-succinimidyl-3-(2-pyridyldithiol)propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

Conjugates of an antibody and one or more small molecule toxins, such as a calicheamicin, maytansinoids, dolastatins, aurostatins, a trichothecene, and CC1065, and the derivatives of these toxins that have toxin activity, are also contemplated herein.

i. Maytansine and Maytansinoids

In some embodiments, the immunoconjugate comprises an antibody (full length or fragments) of the invention conjugated to one or more maytansinoid molecules. Maytansinoids are mitotic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533.

Maytansinoid drug moieties are attractive drug moieties in antibody drug conjugates because they are: (i) relatively accessible to prepare by fermentation or chemical modification, derivatization of fermentation products, (ii) amenable to derivatization with functional groups suitable for conjugation through the non-disulfide linkers to antibodies, (iii) stable in plasma, and (iv) effective against a variety of tumor cell lines.

Immunoconjugates containing maytansinoids, methods of making same, and their therapeutic use are disclosed, for example, in U.S. Pat. Nos. 5,208,020, 5,416,064 and European Patent EP 0 425 235 B1, the disclosures of which are hereby expressly incorporated by reference. Liu et al., Proc. Natl. Acad. Sci. USA 93:8618-8623 (1996) described immunoconjugates comprising a maytansinoid designated DM1 linked to the monoclonal antibody C242 directed against human colorectal cancer. The conjugate was found to be highly cytotoxic towards cultured colon cancer cells, and showed antitumor activity in an in vivo tumor growth assay. Chari et al., Cancer Research 52:127-131 (1992) describe immunoconjugates in which a maytansinoid was conjugated via a disulfide linker to the murine antibody A7 binding to an antigen on human colon cancer cell lines, or to another murine monoclonal antibody TA.1 that binds the HER-2/neu oncogene. The cytotoxicity of the TA.1-maytansinoid conjugate was tested in vitro on the human breast cancer cell line SK-BR-3, which expresses 3.times.10⁵ HER-2 surface antigens per cell. The drug conjugate achieved a degree of cytotoxicity similar to the free maytansinoid drug, which could be increased by increasing the number of maytansinoid molecules per antibody molecule. The A7-maytansinoid conjugate showed low systemic cytotoxicity in mice.

Antibody-maytansinoid conjugates are prepared by chemically linking an antibody to a maytansinoid molecule without significantly diminishing the biological activity of either the antibody or the maytansinoid molecule. See, e.g., U.S. Pat. No. 5,208,020 (the disclosure of which is hereby expressly incorporated by reference). An average of 3-4 maytansinoid molecules conjugated per antibody molecule has shown efficacy in enhancing cytotoxicity of target cells without negatively affecting the function or solubility of the antibody, although even one molecule of toxin/antibody would be expected to enhance cytotoxicity over the use of naked antibody. Maytansinoids are well known in the art and can be synthesized by known techniques or isolated from natural sources. Suitable maytansinoids are disclosed, for example, in U.S. Pat. No. 5,208,020 and in the other patents and nonpatent publications referred to hereinabove. Preferred maytansinoids are maytansinol and maytansinol analogues modified in the aromatic ring or at other positions of the maytansinol molecule, such as various maytansinol esters.

There are many linking groups known in the art for making antibody-maytansinoid conjugates, including, for example, those disclosed in U.S. Pat. No. 5,208,020 or EP Patent 0 425 235 B1, Chari et al., Cancer Research 52:127-131 (1992), and U.S. patent application Ser. No. 10/960,602, filed Oct. 8, 2004, the disclosures of which are hereby expressly incorporated by reference. Antibody-maytansinoid conjugates comprising the linker component SMCC may be prepared as disclosed in U.S. patent application Ser. No. 10/960,602, filed Oct. 8, 2004. The linking groups include disulfide groups, thioether groups, acid labile groups, photolabile groups, peptidase labile groups, or esterase labile groups, as disclosed in the above-identified patents, disulfide and thioether groups being preferred. Additional linking groups are described and exemplified herein.

Conjugates of the antibody and maytansinoid may be made using a variety of bifunctional protein coupling agents such as N-succinimidyl-3-(2-pyridyldithio)propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Particularly preferred coupling agents include N-succinimidyl-3-(2-pyridyldithio)propionate (SPDP) (Carlsson et al., Biochem. J. 173:723-737 (1978)) and N-succinimidyl-4-(2-pyridylthio)pentanoate (SPP) to provide for a disulfide linkage.

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hydroxymethyl, the C-15 position modified with a hydroxyl group, and the C-20 position having a hydroxyl group. In a preferred embodiment, the linkage is formed at the C-3 position of maytansinol or a maytansinol analogue.

ii. Auristatins and Dolastatins

In some embodiments, the immunoconjugate comprises an antibody of the invention conjugated to dolastatins or dolostatin peptidic analogs and derivatives, the auristatins (U.S. Pat. Nos. 5,635,483; 5,780,588). Dolastatins and auristatins have been shown to interfere with microtubule dynamics, GTP hydrolysis, and nuclear and cellular division (Woyke et al (2001) Antimicrob. Agents and Chemother. 45(12):3580-3584) and have anticancer (U.S. Pat. No. 5,663,149) and antifungal activity (Pettit et al (1998) Antimicrob. Agents Chemother. 42:2961-2965). The dolastatin or auristatin drug moiety may be attached to the antibody through the N (amino) terminus or the C (carboxyl) terminus of the peptidic drug moiety (WO 02/088172).

Exemplary auristatin embodiments include the N-terminus linked monomethylauristatin drug moieties DE and DF, disclosed in “Monomethylvaline Compounds Capable of Conjugation to Ligands”, U.S. Ser. No. 10/983,340, filed Nov. 5, 2004, the disclosure of which is expressly incorporated by reference in its entirety.

Typically, peptide-based drug moieties can be prepared by forming a peptide bond between two or more amino acids and/or peptide fragments. Such peptide bonds can be prepared, for example, according to the liquid phase synthesis method (see E. Schroder and K. Lubke, “The Peptides”, volume 1, pp 76-136, 1965, Academic Press) that is well known in the field of peptide chemistry. The auristatin/dolastatin drug moieties may be prepared according to the methods of: U.S. Pat. No. 5,635,483; U.S. Pat. No. 5,780,588; Pettit et al (1989) J. Am. Chem. Soc. 111:5463-5465; Pettit et al (1998) Anti-Cancer Drug Design 13:243-277; Pettit, G. R., et al. Synthesis, 1996, 719-725; and Pettit et al (1996) J. Chem. Soc. Perkin Trans. 1 5:859-863. See also Doronina (2003) Nat Biotechnol 21(7):778-784; “Monomethylvaline Compounds Capable of Conjugation to Ligands”, U.S. Ser. No. 10/983,340, filed Nov. 5, 2004, hereby incorporated by reference in its entirety (disclosing, e.g., linkers and methods of preparing monomethylvaline compounds such as MMAE and MMAF conjugated to linkers).

iii. Calicheamicin

In other embodiments, the immunoconjugate comprises an antibody of the invention conjugated to one or more calicheamicin molecules. The calicheamicin family of antibiotics are capable of producing double-stranded DNA breaks at sub-picomolar concentrations. For the preparation of conjugates of the calicheamicin family, see U.S. Pat. Nos. 5,712,374, 5,714,586, 5,739,116, 5,767,285, 5,770,701, 5,770,710, 5,773,001, 5,877,296 (all to American Cyanamid Company). Structural analogues of calicheamicin which may be used include, but are not limited to γ₁^{I}, α₂^{I}, α₃^{I}, N-acetyl-γ₁^{I}, PSAG and θ^{I}₁ (Hinman et al., Cancer Research 53:3336-3342 (1993), Lode et al., Cancer Research 58:2925-2928 (1998) and the aforementioned U.S. patents to American Cyanamid). Another anti-tumor drug that the antibody can be conjugated is QFA which is an antifolate. Both calicheamicin and QFA have intracellular sites of action and do not readily cross the plasma membrane. Therefore, cellular uptake of these agents through antibody mediated internalization greatly enhances their cytotoxic effects.

iv. Other Cytotoxic Agents

Other antitumor agents that can be conjugated to the antibodies of the invention include BCNU, streptozoicin, vincristine and 5-fluorouracil, the family of agents known collectively LL-E33288 complex described in U.S. Pat. Nos. 5,053,394, 5,770,710, as well as esperamicins (U.S. Pat. No. 5,877,296).

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232 published Oct. 28, 1993.

Procedures for conjugating the biological agents with the cytotoxic agents have been previously described. Procedures for conjugating chlorambucil with antibodies are described by Flechner, I, European Journal of Cancer, 9:741-745 (1973); Ghose, T. et al., British Medical Journal, 3:495-499 (1972); and Szekerke, M., et al., Neoplasma, 19:211-215 (1972), which are hereby incorporated by reference. Procedures for conjugating daunomycin and adriamycin to antibodies are described by Hurwitz, E. et al., Cancer Research, 35:1175-1181 (1975) and Arnon, R. et al. Cancer Surveys, 1:429-449 (1982), which are hereby incorporated by reference. Procedures for preparing antibody-ricin conjugates are described in U.S. Pat. No. 4,414,148 and by Osawa, T., et al. Cancer Surveys, 1:373-388 (1982) and the references cited therein, which are hereby incorporated by reference. Coupling procedures are also described in EP 86309516.2, which is hereby incorporated by reference.

The present invention further contemplates an immunoconjugate formed between an antibody and a compound with nucleolytic activity (e.g., a ribonuclease or a DNA endonuclease such as a deoxyribonuclease; DNase).

For selective destruction of the tumor, the antibodies of the invention can be coupled to high energy radiation emitters, for example, a radioisotope, such as ¹³¹I, a γ-emitter, which, when localized at the tumor site, results in a killing of several cell diameters. See, e.g., S. E. Order, “Analysis, Results, and Future Prospective of the Therapeutic Use of Radiolabeled Antibody in Cancer Therapy”, Monoclonal Antibodies for Cancer Detection and Therapy, R. W. Baldwin et al. (eds.), pp 303-316 (Academic Press 1985), which is hereby incorporated by reference. A variety of radioactive isotopes are available for the production of radioconjugated antibodies. Examples include At₂₁₁, I¹³¹, I¹²⁵, Y⁹⁰, Re^{186,} Re¹⁸⁸, Sm¹⁵³, Bi²¹², P³², Pb²¹² and radioactive isotopes of Lu. When the conjugate is used for detection, it may comprise a radioactive atom for scintigraphic studies, for example tc⁹⁹m or I¹²³, or a spin label for nuclear magnetic resonance (NMR) imaging (also known as magnetic resonance imaging, MRI), such as iodine-123 again, iodine-131, indium-111, fluorine-19, carbon-13, nitrogen-15, oxygen-17, gadolinium, manganese or iron.

The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as tc⁹⁹m or I¹²³, Re¹⁸⁶, Re¹⁸⁸ and In¹¹¹ can be attached via a cysteine residue in the peptide. Yttrium-90 can be attached via a lysine residue. The IODOGEN method (Fraker et al (1978) Biochem. Biophys. Res. Commun. 80: 49-57) can be used to incorporate iodine-123. “Monoclonal Antibodies in Immunoscintigraphy” (Chatal, CRC Press 1989) describes other methods in detail.

Conjugates of the antibody and cytotoxic agent may be made using a variety of bifunctional protein coupling agents such as N-succinimidyl-3-(2-pyridyldithio)propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell, For example, an acid-labile linker, peptidase-sensitive linker, photolabile linker, dimethyl linker or disulfide-containing linker (Chari et al., Cancer Research 52:127-131 (1992); U.S. Pat. No. 5,208,020) may be used.

The compounds of the invention expressly contemplate, but are not limited to, ADC prepared with cross-linker reagents: BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A). See pages 467-498, 2003-2004 Applications Handbook and Catalog.

v. Preparation of Antibody Drug Conjugates

In the antibody drug conjugates (ADC) of the invention, an antibody (Ab) is conjugated to one or more drug moieties (D), e.g. about 1 to about 20 drug moieties per antibody, through a linker (L). The ADC of Formula I may be prepared by several routes, employing organic chemistry reactions, conditions, and reagents known to those skilled in the art, including: (1) reaction of a nucleophilic group of an antibody with a bivalent linker reagent, to form Ab-L, via a covalent bond, followed by reaction with a drug moiety D; and (2) reaction of a nucleophilic group of a drug moiety with a bivalent linker reagent, to form D-L, via a covalent bond, followed by reaction with the nucleophilic group of an antibody. Additional methods for preparing ADC are described herein.

### Ab-(L-D)ₚ I

The linker may be composed of one or more linker components. Exemplary linker components include 6-maleimidocaproyl (“MC”), maleimidopropanoyl (“MP”), valine-citrulline (“val-cit”), alanine-phenylalanine (“ala-phe”), p-aminobenzyloxycarbonyl (“PAB”), N-Succinimidyl 4-(2-pyridylthio) pentanoate (“SPP”), N-Succinimidyl 4-(N-maleimidomethyl)cyclohexane-1 carboxylate (“SMCC”), and N-Succinimidyl (4-iodo-acetyl)aminobenzoate (“SIAB”). Additional linker components are known in the art and some are described herein. See also “Monomethylvaline Compounds Capable of Conjugation to Ligands”, U.S. Ser. No. 10/983,340, filed Nov. 5, 2004, the contents of which are hereby incorporated by reference in its entirety.

In some embodiments, the linker may comprise amino acid residues. Exemplary amino acid linker components include a dipeptide, a tripeptide, a tetrapeptide or a pentapeptide. Exemplary dipeptides include: valine-citrulline (vc or val-cit), alanine-phenylalanine (af or ala-phe). Exemplary tripeptides include: glycine-valine-citrulline (gly-val-cit) and glycine-glycine-glycine (gly-gly-gly). Amino acid residues which comprise an amino acid linker component include those occurring naturally, as well as minor amino acids and non-naturally occurring amino acid analogs, such as citrulline. Amino acid linker components can be designed and optimized in their selectivity for enzymatic cleavage by a particular enzyme, for example, a tumor-associated protease, cathepsin B, C and D, or a plasmin protease.

Nucleophilic groups on antibodies include, but are not limited to: (i) N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups. Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by introducing one, two, three, four, or more cysteine residues (e.g., preparing mutant antibodies comprising one or more non-native cysteine amino acid residues).

Antibody drug conjugates of the invention may also be produced by modification of the antibody to introduce electrophilic moieties, which can react with nucleophilic substituents on the linker reagent or drug. The sugars of glycosylated antibodies may be oxidized, e.g. with periodate oxidizing reagents, to form aldehyde or ketone groups which may react with the amine group of linker reagents or drug moieties. The resulting imine Schiff base groups may form a stable linkage, or may be reduced, e.g. by borohydride reagents to form stable amine linkages. In one embodiment, reaction of the carbohydrate portion of a glycosylated antibody with either glactose oxidase or sodium meta-periodate may yield carbonyl (aldehyde and ketone) groups in the protein that can react with appropriate groups on the drug (Hermanson, Bioconjugate Techniques). In another embodiment, proteins containing N-terminal serine or threonine residues can react with sodium meta-periodate, resulting in production of an aldehyde in place of the first amino acid (Geoghegan & Stroh, (1992) Bioconjugate Chem. 3:138-146; U.S. Pat. No. 5,362,852). Such aldehyde can be reacted with a drug moiety or linker nucleophile.

Likewise, nucleophilic groups on a drug moiety include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups.

Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

In yet another embodiment, the antibody may be conjugated to a “receptor” (such streptavidin) for utilization in tumor pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

### RNAi

In certain embodiments, the cytotoxic agent is a gene modifier, e.g, RNAi molecule. Methods for making specific RNAi (RNA interference) nucleic acids are described in the art (see, e.g., U.S. Pat. No. 6,506,559; WO 01/75164; WO 99/32619; Elbashir et al., Nature 411:494-98 (2001); Zhang et al., Curr. Pharm. Biotech. 5:1-7 (2004); Paddison et al., Curr. Opin. Mol. Ther. 5:217-24 (2003).

RNA interference refers to the process of sequence-specific post transcriptional gene silencing in animals mediated by short interfering RNAs (siRNA) (Fire et al., 1998, Nature, 391, 806). The corresponding process in plants is commonly referred to as post transcriptional gene silencing or RNA silencing and is also referred to as quelling in fungi. The process of post transcriptional gene silencing is thought to be an evolutionarily conserved cellular defense mechanism used to prevent the expression of foreign genes which is commonly shared by diverse flora and phyla (Fire et al., 1999, Trends Genet., 15, 358). Such protection from foreign gene expression may have evolved in response to the production of double stranded RNAs (dsRNA) derived from viral infection or the random integration of transposon elements into a host genome via a cellular response that specifically destroys homologous single stranded RNA or viral genomic RNA. The presence of dsRNA in cells triggers the RNAi response though a mechanism that has yet to be fully characterized. This mechanism appears to be different from the interferon response that results from dsRNA mediated activation of protein kinase PKR and 2′,5′-oligoadenylate synthetase resulting in non-specific cleavage of mRNA by ribonuclease L.

The presence of long dsRNAs in cells stimulates the activity of a ribonuclease III enzyme referred to as dicer. Dicer is involved in the processing of the dsRNA into short pieces of dsRNA known as short interfering RNAs (siRNA) (Berstein et al., 2001, Nature, 409, 363). Short interfering RNAs derived from dicer activity are typically about 21-23 nucleotides in length and comprise about 19 base pair duplexes. Dicer has also been implicated in the excision of 21 and 22 nucleotide small temporal RNAs (stRNA) from precursor RNA of conserved structure that are implicated in translational control (Hutvagner et al., 2001, Science, 293, 834). The RNAi response also features an endonuclease complex containing a siRNA, commonly referred to as an RNA-induced silencing complex (RISC), which mediates cleavage of single stranded RNA having sequence complementary to the antisense strand of the siRNA duplex. Cleavage of the target RNA takes place in the middle of the region complementary to the antisense strand of the siRNA duplex (Elbashir et al., 2001, Genes Dev., 15, 188).

### Pharmaceutical Formulations

Therapeutic formulations comprising an antibody of the invention are prepared for storage by mixing the antibody having the desired degree of purity with optional physiologically acceptable carriers, excipients or stabilizers (Remington: The Science and Practice of Pharmacy 20th edition (2000)), in the form of aqueous solutions, lyophilized or other dried formulations. Acceptable carriers, excipients, or stabilizers are nontoxic to recipients at the dosages and concentrations employed, and include buffers such as phosphate, citrate, histidine and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington: The Science and Practice of Pharmacy 20th edition (2000).

The formulations to be used for in vivo administration must be sterile. This is readily accomplished by filtration through sterile filtration membranes.

### Therapeutic and Non-Therapeutic Uses

Any one or more of the anti-Notch1 antibodies described herein can be used in a method for binding an antigen, preferably Notch1 receptor protein in a subject suffering from a disorder associated with increased antigen expression and/or activity, comprising administering to the subject an antibody of the invention such that the antigen in the subject is bound. Preferably, the antigen is a human protein molecule and the subject is a human subject. Consequently, an antibody of the invention can be administered to a human subject for therapeutic purposes. As well, an antibody of the invention can be administered to a non-human mammal expressing an antigen with which the immunoglobulin cross-reacts (e.g., a primate, pig or mouse) for veterinary purposes or as an animal model of human disease. Regarding the latter, such animal models may be useful for evaluating the therapeutic efficacy of antibodies of the invention (e.g., testing of dosages and time courses of administration).

The present Notch receptor agonist, antagonist and neutralizing antibodies are useful as therapeutic reagents for treating a Notch receptor expressing cancer or alleviating one or more symptoms of the cancer in a mammal. The antibodies of the invention can also be used to treat other Notch-mediated disorders such as inflamatory disorders etc. The antibody is able to bind to at least a portion of the cancer cells that express a Notch receptor in the mammal and preferably destroy or kill Notch receptor-expressing tumor cells or inhibit the growth of such tumor cells, in vitro, ex vivo or in vivo. Such an antibody include a naked anti-Notch receptor antibody (not conjugated to any agent). Naked antibodies that have cytotoxic or cell growth inhibition properties can be further harnessed with a cytotoxic agent to render them even more potent in tumor cell destruction. Cytotoxic properties can be conferred to an anti-Notch receptor antibody by, e.g., conjugating the antibody with a cytotoxic agent, to form an immunoconjugate as described herein.

In one aspect, the invention provides methods for treating or preventing a tumor, a cancer, and/or a cell proliferative disorder associated with increased expression and/or activity of Notch1, the methods comprising administering an effective amount of an anti-Notch1 antibody to a subject in need of such treatment.

In one aspect, the invention provides methods for reducing, inhibiting, blocking, or preventing growth of a tumor or cancer, the methods comprising administering an effective amount of an anti-Notch1 antibody to a subject in need of such treatment.

In one aspect, the invention provides methods for treating a tumor, a cancer, and/or a cell proliferative disorder comprising administering an effective amount of an anti-Notch1 antibody to a subject in need of such treatment.

In one aspect, the invention provides methods for inhibiting cellular proliferative disorders including angiogenesis comprising administering an effective amount of an anti-Notch1 antibody to a subject in need of such treatment.

In one aspect, the invention provides methods for treating a pathological condition associated with a cellular proliferative disorder comprising administering an effective amount of an anti-Notch1 antibody to a subject in need of such treatment. In some embodiments, the pathological condition associated with said cellular proliferative disorder is a tumor and/or cancer.

The antibodies in accordance with the present invention may be used to deliver a variety of cytotoxic drugs including therapeutic drugs, a compound emitting radiation, molecules of plants, fungal, or bacterial origin, biological proteins, and mixtures thereof. The cytotoxic drugs can be intracellularly acting cytotoxic drugs, such as short-range radiation emitters, including, for example, short-range, high-energy α-emitters.

The antibodies of the invention can be used to treat, inhibit, delay progression of, prevent/delay recurrence of, ameliorate, or prevent diseases, disorders or conditions associated with expression and/or activity of one or more antigen molecules.

Exemplary disorders include breast cancer, lung cancer, colorectal cancer, pancreatic cancer, prostate cancer, and head and neck cancer. Also included are Notch-receptor mediated leukemias, disorders involving neovascularization, cadiovascular disease associated with Notch1 polymorphism (US 2006/0134645) and plasma disorders as described in US 2005/0129686.

In certain embodiments, an immunoconjugate comprising an antibody conjugated with one or more cytotoxic agent(s) is administered to the patient. In some embodiments, the immunoconjugate and/or antigen to which it is bound is/are internalized by the cell, resulting in increased therapeutic efficacy of the immunoconjugate in killing the target cell to which it binds. In one embodiment, the cytotoxic agent targets or interferes with nucleic acid in the target cell. In one embodiment, the cytotoxic agent targets or interferes with microtubule polymerization. Examples of such cytotoxic agents include any of the chemotherapeutic agents noted herein (such as a maytansinoid, auristatin, dolastatin, or a calicheamicin), a radioactive isotope, or a ribonuclease or a RNAi or a DNA endonuclease.

The anti-Notch1 antibodies or immunoconjugates are administered to a human patient, in accord with known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. Intravenous or subcutaneous administration of the antibody is preferred.

Other therapeutic regimens may be combined with the administration of the anti-Notch receptor antibody. The combined administration includes co-administration, using separate formulations or a single pharmaceutical formulation, and consecutive administration in either order, wherein preferably there is a time period while both (or all) active agents simultaneously exert their biological activities. Preferably such combined therapy results in a synergistic therapeutic effect.

It may also be desirable to combine administration of the anti-Notch receptor antibody or antibodies, with administration of an antibody directed against another tumor antigen associated with the particular cancer. In another embodiment, the invention antibody is a bispecific construct targeting two distinct epitopes. The epitopes may be on the same antigen or on separate antigens, one of which is Notch1 receptor protein.

Antibodies of the invention can be used either alone or in combination with other compositions in a therapy. For instance, an antibody of the invention may be co-administered with another antibody, chemotherapeutic agent(s) (including cocktails of chemotherapeutic agents), other cytotoxic agent(s), anti-angiogenic agent(s), cytokines, and/or growth inhibitory agent(s). Where an antibody of the invention inhibits tumor growth, it may be particularly desirable to combine it with one or more other therapeutic agent(s) which also inhibits tumor growth, e.g. anti-EGFR agents including antibodies to any one or more of the EGFR family of receptors. Alternatively, or additionally, the patient may receive combined radiation therapy (e.g. external beam irradiation or therapy with a radioactive labeled agent, such as an antibody). Such combined therapies noted above include combined administration (where the two or more agents are included in the same or separate formulations), and separate administration, in which case, administration of the antibody of the invention can occur prior to, and/or following, administration of the adjunct therapy or therapies.

It is to be further understood that a cocktail of different monoclonal antibodies, such as a mixture of the specific monoclonal antibodies described herein, or their binding fragments, may be administered, if necessary or desired, for cancer treatment. Indeed, using a mixture of monoclonal antibodies, or binding fragments thereof, in a cocktail to target several antigens, or different epitopes, on cancer cells, is an advantageous approach, particularly to prevent evasion of tumor cells and/or cancer cells due to down regulation of one of the antigens.

### Combination Therapies

As indicated above, the invention provides combined therapies in which an anti-Notch1 antibody is administered with another therapy. For example, anti-Notch1 antibodies are used in combinations with anti-cancer therapeutics or anti-neovascularization therapeutics to treat various neoplastic or non-neoplastic conditions. In one embodiment, the neoplastic or non-neoplastic condition is characterized by pathological disorder associated with aberrant or undesired angiogenesis. The anti-Notch1 antibody can be administered serially or in combination with another agent that is effective for those purposes, either in the same composition or as separate compositions. Alternatively, or additionally, multiple inhibitors of Notch1 can be administered.

The administration of the anti-Notch1 antibody can be done simultaneously, e.g., as a single composition or as two or more distinct compositions using the same or different administration routes. Alternatively, or additionally, the administration can be done sequentially, in any order. In certain embodiments, intervals ranging from minutes to days, to weeks to months, can be present between the administrations of the two or more compositions. For example, the anti-cancer agent may be administered first, followed by the Notch1 inhibitor. However, simultaneous administration or administration of the anti-Notch1 antibody first is also contemplated.

The effective amounts of therapeutic agents administered in combination with an anti-Notch1 antibody will be at the physician's or veterinarian's discretion. Dosage administration and adjustment is done to achieve maximal management of the conditions to be treated. The dose will additionally depend on such factors as the type of therapeutic agent to be used and the specific patient being treated. Suitable dosages for the anti-cancer agent are those presently used and can be lowered due to the combined action (synergy) of the anti-cancer agent and the anti-Notch1 antibody. In certain embodiments, the combination of the inhibitors potentiates the efficacy of a single inhibitor. The term “potentiate” refers to an improvement in the efficacy of a therapeutic agent at its common or approved dose. See also the section entitled “Pharmaceutical Compositions” herein.

Typically, the anti-Notch1 antibodies and anti-cancer agents are suitable for the same or similar diseases to block or reduce a pathological disorder such as tumor growth or growth of a cancer cell. In one embodiment the anti-cancer agent is an anti-angiogenesis agent.

“Hyper cellular proliferative” therapy in relationship to cancer is a cancer treatment strategy aimed at inhibiting the neoplastic growth of tumor cells expressing the Notch1 receptor protein as well as preventing metastasis of tumors at the secondary sites, therefore allowing attack of the tumors by other therapeutics.

The anti-Nocth antibodies may also be used in addition to or in conjunction with anti-angiogenic agents that inhibit excessive tumor vascular development. Consequently, hyper-cellular proliferative therapy contemplated herein may be combined with an “antiangiogenic” therapy comprising anti-angiogenic compounds, of which many have been identified and are known in the arts, including those listed herein, e.g., listed under Definitions, and by, e.g., Carmeliet and Jain, Nature 407:249-257 (2000); Ferrara et al., Nature Reviews. Drug Discovery, 3:391-400 (2004); and Sato Int. J. Clin. Oncol., 8:200-206 (2003). See also, US Patent Application US20030055006. In one embodiment, an anti-Notch1 antibody is used in combination with an anti-VEGF neutralizing antibody (or fragment) and/or another VEGF antagonist or a VEGF receptor antagonist including, but not limited to, for example, soluble VEGF receptor (e.g., VEGFR-1, VEGFR-2, VEGFR-3, neuropillins (e.g., NRP1, NRP2)) fragments, aptamers capable of blocking VEGF or VEGFR, neutralizing anti-VEGFR antibodies, low molecule weight inhibitors of VEGFR tyrosine kinases (RTK), antisense strategies for VEGF, ribozymes against VEGF or VEGF receptors, antagonist variants of VEGF; and any combinations thereof. Alternatively, or additionally, two or more angiogenesis inhibitors may optionally be co-administered to the patient in addition to VEGF antagonist and other agent. In certain embodiment, one or more additional therapeutic agents, e.g., anti-cancer agents, can be administered in combination with anti-Notch1 antibody, the VEGF antagonist, and an anti-angiogenesis agent.

In certain aspects of the invention, other therapeutic agents useful for combination tumor therapy with an anti-Notch1 antibody include other cancer therapies, (e.g., surgery, radiological treatments (e.g., involving irradiation or administration of radioactive substances), chemotherapy, treatment with anti-cancer agents listed herein and known in the art, or combinations thereof). Alternatively, or additionally, two or more antibodies binding the same or two or more different antigens disclosed herein can be co-administered to the patient. Sometimes, it may be beneficial to also administer one or more cytokines to the patient.

Yet another embodiment provides a method for treating an Notch1 mediated cancer comprising: a) obtaining a sample of diseased tissue from a patient in need of treatment of said cancer; b) determining the level of expression of Notch1 levels in the tissue sample; c) scoring the samples for expression of Notch1 levels; d) correlating the score to identify patients likely to benefit from treatment with an Notch1 antagonist, wherein the step of correlating comprises comparing said scoring to that obtained from a control sample, e) treating the patient with a therapeutic regime known to improve the prognosis for the particular cancer. In certain embodiments, the method further proposes f) repeating steps “a” and “b”, and g) adjusting the therapeutic regime known to improve the prognosis for the cancer; h) repeating steps a-f as frequently as deemed appropriate. Exemplary and non-limiting methods of “detecting” Notch1 expression for staging or scoring purposes is provided herebelow. Refer to, for example, section marked “Detection of Notch1 antigen.”

Additional combination therapies include combining any one or more of the invention antibodies with glucocorticoids (US 20060003927) and gamma-secretase inhibitors (20080206753 including references cited therein e.g., Lanz, T. A., Hosley, J. D., Adams, W. J., and Merchant, K. M. 2004. Studies of Abeta pharmacodynamics in the brain, cerebrospinal fluid, and plasma in young (plaque-free) Tg2576 mice using the gamma-secretase inhibitor N2-[(2S)-2-(3,5-difluorophenyl)-2-hydroxyethanoyl]-N1-[(7S)-5-methyl-6-ox-o-6,7-dihydro-5H-dibenzo[b,d]azepin-7-yl]-alaninamide (LY-411575). J Pharmacol Exp Ther 309:49-55.)

### Chemotherapeutic Agents

In certain aspects, the invention provides a method of blocking or reducing tumor growth or growth of a cancer cell, by administering effective amounts of an antagonist of Notch1 and/or an angiogenesis inhibitor(s) and one or more chemotherapeutic agents to a patient susceptible to, or diagnosed with, cancer. A variety of chemotherapeutic agents may be used in the combined treatment methods of the invention. An exemplary and non-limiting list of chemotherapeutic agents contemplated is provided herein under “Definitions.”

Likewise, in certain aspects, the invention provides for the use of “agonist” anti-Notch1 antibodies.

As will be understood by those of ordinary skill in the art, the appropriate doses of chemotherapeutic agents will be generally around those already employed in clinical therapies wherein the chemotherapeutics are administered alone or in combination with other chemotherapeutics. Variation in dosage will likely occur depending on the condition being treated. The physician administering treatment will be able to determine the appropriate dose for the individual subject.

The invention also provides methods and compositions for inhibiting or preventing relapse tumor growth or relapse cancer cell growth. Relapse tumor growth or relapse cancer cell growth is used to describe a condition in which patients undergoing or treated with one or more currently available therapies (e.g., cancer therapies, such as chemotherapy, radiation therapy, surgery, hormonal therapy and/or biological therapy/immunotherapy, anti-VEGF antibody therapy, particularly a standard therapeutic regimen for the particular cancer) is not clinically adequate to treat the patients or the patients are no longer receiving any beneficial effect from the therapy such that these patients need additional effective therapy. As used herein, the phrase can also refer to a condition of the “non-responsive/refractory” patient, e.g., which describe patients who respond to therapy yet suffer from side effects, develop resistance, do not respond to the therapy, do not respond satisfactorily to the therapy, etc. In various embodiments, a cancer is relapse tumor growth or relapse cancer cell growth where the number of cancer cells has not been significantly reduced, or has increased, or tumor size has not been significantly reduced, or has increased, or fails any further reduction in size or in number of cancer cells. The determination of whether the cancer cells are relapse tumor growth or relapse cancer cell growth can be made either in vivo or in vitro by any method known in the art for assaying the effectiveness of treatment on cancer cells, using the art-accepted meanings of “relapse” or “refractory” or “non-responsive” in such a context. A tumor resistant to anti-EGFR treatment is an example of a relapse tumor growth.

The invention provides methods of blocking or reducing relapse tumor growth or relapse cancer cell growth in a subject by administering one or more anti-Notch1 antibodies to block or reduce the relapse tumor growth or relapse cancer cell growth in subject. In certain embodiments, the Notch1 antagonist or agonist antibodies in accordance with the invention can be administered subsequent to the cancer therapeutic. In certain embodiments, the anti-Notch1 antibodies are administered simultaneously with cancer therapy. Alternatively, or additionally, the anti-Notch1 antibody therapy alternates with another cancer therapy, which can be performed in any order. The invention also encompasses methods for administering one or more inhibitory antibodies to prevent the onset or recurrence of cancer in patients predisposed to having cancer. Generally, the subject was or is concurrently undergoing cancer therapy. In one embodiment, the cancer therapy is treatment with an anti-angiogenesis agent, e.g., a VEGF antagonist. In one embodiment, the anti-angiogenesis agent is an anti-VEGF neutralizing antibody or fragment (e.g. AVASTIN™ (Genentech, South San Francisco, Calif.) or LUCENTIS™ (Genentech, South San Francisco, Calif.)), Y0317, M4, G6, B20, 2C3, etc.). See, e.g., U.S. Pat. Nos. 6,582,959, 6,884,879, 6,703,020; WO98/45332; WO 96/30046; WO94/10202; EP 0666868B1; US Patent Applications 20030206899, 20030190317, 20030203409, and 20050112126; Popkov et al., Journal of Immunological Methods 288:149-164 (2004); and, WO2005012359. Additional agents can be administered in combination with VEGF antagonist and an anti-Notch1 antibody for blocking or reducing relapse tumor growth or relapse cancer cell growth, e.g., see section entitled Combination Therapies herein.

The antibody of the invention (and adjunct therapeutic agent) is/are administered by any suitable means, including parenteral, subcutaneous, intraperitoneal, intrapulmonary, and intranasal, and, if desired for local treatment, intralesional or intravitreal administration. Parenteral infusions include intramuscular, intravenous, intraarterial, intraperitoneal, or subcutaneous administration. In addition, the antibody is suitably administered by pulse infusion, particularly with declining doses of the antibody. Dosing can be by any suitable route, e.g. by injections, such as intravenous or subcutaneous injections, depending in part on whether the administration is brief or chronic.

The antibody composition of the invention will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibodies of the invention present in the formulation, the type of disorder or treatment, and other factors discussed above. These are generally used in the same dosages and with administration routes as used hereinbefore or about from 1 to 99% of the heretofore employed dosages.

For the prevention or treatment of disease, the appropriate dosage of an antibody of the invention (when used alone or in combination with other agents such as chemotherapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g. 0.1 mg/kg-10 mg/kg) of antibody is an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, e.g. about six doses of the antibody). An initial higher loading dose, followed by one or more lower doses may be administered. An exemplary dosing regimen comprises administering an initial loading dose of about 4 mg/kg, followed by a weekly maintenance dose of about 2 mg/kg of the antibody. However, other dosage regimens may be useful. The progress of this therapy is easily monitored by conventional techniques and assays.

### Detection of Notch1 Antigen

It is well accepted that cell surface growth receptor proteins, especially those whose expression correlates with an oncogenic disorder, e.g., Notch1 are excellent targets for drug candidates or tumor (e.g., cancer) treatment. The state of the art now concludes that such proteins may also find use in non-therapeutic applications. The exquisite specificity of the anti-Notch antibodies for the Notch1 receptor protein can be exploited for various uses including diagnostic and prognostic reagents. The proposed uses exploit the observation that (i) the anti-Notch1 antibodies of the invention including antigen binding fragments thereof specifically bind Notch1 and (ii) the target receptor bound by the antibodies of the invention is highly expressed on cancerous cells. Expressly contemplated also are the use of the invention antibodies in detecting, monitoring, and quantifying Notch1 expression (e.g. in an ELISA or a Western blot); purification or immunoprecipitation of Notch1 from cells, to kill and eliminate Notch1-expressing cells from a population of mixed cells as a step in the purification of other cells. Proposed methods of diagnosis can be performed in vitro using a cellular sample (e.g.,lymph node biopsy or tissue) from a patient or be performed by in vivo imaging. Diagnostic and prognostic applications include scoring tumors as well as staging Notch1-expressing cancers (e.g., in radioimaging). They may be used alone or in combination with other Notch1 related cancer markers. The diagnostic uses of the antibodies according to the present invention embrace primary tumors and cancers, as well as metastases. Other cancers and tumors bearing the antigen are also amenable to these diagnostic and imaging procedures.

In one embodiment, the invention antibody or binding fragments thereof will be very useful in cancer diagnosis and prognosis by effectively allowing one skilled in the art to quantitate or quantify the expression levels of Notch1 in whatever kind of “sample” it may occur. This can be achieved, for example, by immunofluorescence techniques employing a fluorescently labeled antibody, coupled with light microscopic, flow cytometric, or fluorometric detection. In addition, the antibodies, or binding fragments thereof, according to the present invention may additionally be employed histologically, as in immunofluorescence, immunoelectron microscopy, or non-immuno assays, for in situ detection of the cancer-specific antigen on cells, such as for use in monitoring, diagnosing, or detection assays. See, for example, Zola, Monoclonal Antibodies: A Manual of Techniques, pp. 147 158 (CRC Press, Inc. 1987).

In another aspect, the invention provides methods for detection of Notch1, the methods comprising detecting Notch1-anti-Notch1 antibody complex in the sample. The term “detection” as used herein includes qualitative and/or quantitative detection (measuring levels) with or without reference to a control.

In another aspect, the invention provides methods for diagnosing a disorder associated with Notch1 expression and/or activity, the methods comprising detecting Notch1-anti-Notch1 antibody complex in a biological sample from a patient having or suspected of having the disorder. In some embodiments, the Notch1 expression is increased expression or abnormal (undesired) expression. In some embodiments, the disorder is a tumor, cancer, and/or a cell proliferative disorder.

In another aspect, the invention provides any of the anti-Notch1 antibodies described herein, wherein the anti-Notch1 antibody comprises a detectable label.

In another aspect, the invention provides a complex of any of the anti-Notch1 antibodies described herein and Notch1. In some embodiments, the complex is in vivo or in vitro. In some embodiments, the complex comprises a cancer cell. In some embodiments, the anti-Notch1 antibody is detectably labeled.

Anti-Notch1 antibodies can be used for the detection of Notch1 in any one of a number of well known detection assay methods. For example, a biological sample may be assayed for Notch1 by obtaining the sample from a desired source, admixing the sample with anti-Notch1 antibody to allow the antibody to form antibody/Notch1 complex with any Notch1 present in the mixture, and detecting any antibody/Notch1 complex present in the mixture. The biological sample may be prepared for assay by methods known in the art which are suitable for the particular sample. The methods of admixing the sample with antibodies and the methods of detecting antibody/Notch1 complex are chosen according to the type of assay used. Such assays include immunohistochemistry, competitive and sandwich assays, and steric inhibition assays.

Analytical methods for Notch1 all use one or more of the following reagents: labeled Notch1 analogue, immobilized Notch1 analogue, labeled anti-Notch1 antibody, immobilized anti-Notch1 antibody and steric conjugates. The labeled reagents also are known as “tracers.”

For diagnostic and imaging applications, the antibodies of the invention may be labeled. The label used is any detectable functionality that does not interfere with the binding of Notch1 and anti-Notch1 antibody and it can bind to the antibodies by means of physical binding, chemical binding or the like, thus allowing them to be detected. Numerous labels are known for use in immunoassay, examples including moieties that may be detected directly, such as fluorochrome, chemiluminescent, and radioactive labels, as well as moieties, such as enzymes, that must be reacted or derivatized to be detected. Specific examples of labeling substances include enzymes, fluorescent substances, chemiluminescent substances, biotin, avidin, radioactive isotopes and the like. When the fluorescently labeled antibody is exposed to light of the proper wavelength, its presence can then be detected due to fluorescence. The radioactive isotopes and fluorescent substances detailed herein independently produce detectable signals, but the enzymes, chemiluminescent substances, biotin and avidin do not independently produce detectable signals, but instead produce detectable signals when they react with at least one other substance. For example, in the case of an enzyme at least a substrate is required, and a variety of substrates are used depending on the method of measuring enzyme activity (colorimetry, fluorescence method, bioluminescence method or chemoluminescence method). In the case of biotin generally at least avidin or enzyme-modified avidin is reacted. A variety of colorants dependent on the substrate can also be used as necessary.

Examples of such labels include: Examples of such labels include the radioisotopes ³²P, ¹⁴C, ¹²⁵I ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like. The label may be directly conjugated to the antibodies or fragments thereof or indirectly conjugated. Indeed, numerous ways to detectably label protein molecules are known and practiced in the art. Means of indirect conjugation of a protein to a label are also well known. Indirect conjugation of the label to the antibody may, for example, be achieved by conjugating antibody to a small hapten (e.g., digoxin) and one of the different types of labels mentioned herein is conjugated with an anti-hapten antibody mutant (e.g., anti-digoxin antibody). See, e.g., Wagner et al., J. Nucl. Med. 20: 428 (1979) and Saha et al., J. Nucl. Med. 6:542 (1976), hereby incorporated by reference.

Conventional methods are available to bind these labels covalently to proteins or polypeptides. For instance, coupling agents such as dialdehydes, carbodiimides, dimaleimides, bis-imidates, bis-diazotized benzidine, and the like may be used to tag the antibodies with the above-described fluorescent, chemiluminescent, and enzyme labels. See, for example, U.S. Pat. No. 3,940,475 (fluorimetry) and U.S. Pat. No. 3,645,090 (enzymes); Hunter et al., Nature, 144: 945 (1962); David et al., Biochemistry, 13: 1014-1021 (1974); Pain et al., J. Immunol. Methods, 40: 219-230 (1981); and Nygren, J. Histochem. and Cytochem., 30: 407-412 (1982). Preferred labels herein are enzymes such as horseradish peroxidase and alkaline phosphatase. The conjugation of such label, including the enzymes, to the antibody is a standard manipulative procedure for one of ordinary skill in immunoassay techniques. See, for example, O'Sullivan et al., “Methods for the Preparation of Enzyme-antibody Conjugates for Use in Enzyme Immunoassay,” in Methods in Enzymology, ed. J. J. Langone and H. Van Vunakis, Vol. 73 (Academic Press, New York, N.Y., 1981), pp. 147-166.

Another way to label the antibodies of the invention is by linking the antibody to an enzyme, e.g., for use in an enzyme immunoassay (EIA), (A. Voller et al., 1978, “The Enzyme Linked Immunosorbent Assay (ELISA)”, Diagnostic Horizons, 2:1 7; Microbiological Associates Quarterly Publication, Walkersville, Md.; A. Voller et al., 1978, J. Clin. Pathol., 31:507 520; J. E. Butler et al., 1981, Meths. Enzymol., 73:482 523; Enzyme Immunoassay, 1980, (Ed.) E. Maggio, CRC Press, Boca Raton, Fla.; Enzyme Immunoassay, 1981, (Eds.) E. Ishikawa et al., Kgaku Shoin, Tokyo, Japan). The enzyme that is bound to the antibody reacts with an appropriate substrate, preferably a chromogenic substrate, so as to produce a chemical moiety which can be detected, for example, by spectrophotometric, fluorometric, or by visual detection means. Nonlimiting examples of enzymes which can be used to detectably label the antibodies include malate dehydrogenase, staphylococcal nuclease, delta-5-steroid isomerase, yeast alcohol dehydrogenase, alpha-glycerophosphate dehydrogenase, triose phosphate isomerase, horseradish peroxidase, alkaline phosphatase, ribonuclease, urease, catalase, glucose-6-phosphate dehydrogenase, glucoamylase and acetylcholinesterase. The detection can be accomplished by calorimetric methods, which employ a chromogenic substrate for the enzyme, or by visual comparison of the extent of enzymatic reaction of a substrate compared with similarly prepared standards or controls. Numerous other enzyme-substrate combinations are available to those skilled in the art. For a general review of these, see U.S. Pat. Nos. 4,275,149 and 4,318,980.

Techniques for conjugating enzymes to antibodies are described in O′Sullivan et al., Methods for the Preparation of Enzyme-Antibody Conjugates for use in Enzyme Immunoassay, in Methods in Enzym. (ed J. Langone & H. Van Vunakis), Academic press, New York, 73:147-166 (1981).

Examples of enzyme-substrate combinations include, for example:

(i) Horseradish peroxidase (HRPO) with hydrogen peroxidase as a substrate, wherein the hydrogen peroxidase oxidizes a dye precursor (e.g., orthophenylene diamine (OPD) or 3,3′,5,5′-tetramethyl benzidine hydrochloride (TMB));

(ii) alkaline phosphatase (AP) with para-Nitrophenyl phosphate as chromogenic substrate; and

(iii) β-D-galactosidase (β-D-Gal) with a chromogenic substrate (e.g., p-nitrophenyl-β-D-galactosidase) or fluorogenic substrate 4-methylumbelliferyl-β-D-galactosidase.

Immobilization of reagents is required for certain assay methods. Immobilization entails separating the anti-Notch1 antibody from any Notch1 that remains free in solution. This conventionally is accomplished by either insolubilizing the anti-Notch1 antibody or Notch1 analogue before the assay procedure, as by adsorption to a water-insoluble matrix or surface (Bennich et al., U.S. Pat. No. 3,720,760), by covalent coupling (for example, using glutaraldehyde cross-linking), or by insolubilizing the anti-Notch1 antibody or Notch1 analogue afterward, e.g., by immunoprecipitation.

Suitable subjects include those who are suspected of being at risk of a pathological effect of any hyperproliferative oncogenic disorders, particularly carcinoma and sarcomas mediated by Notch1, are suitable for the detection, diagnosis and prognosis paradigms of the invention. Those with a history of cancer are especially suitable. Suitable human subjects for the diagnostic and prognostic therapies may comprise two groups, which can be distinguished by clinical criteria. Patients with “advanced disease” or “high tumor burden” are those who bear a clinically measurable tumor. A clinically measurable tumor is one that can be detected on the basis of tumor mass (e.g., by palpation, CAT scan, or X-Ray; positive biochemical or histopathological markers on their own may be insufficient to identify this population).

A second group of suitable subjects is known in the art as the “adjuvant group”. These are individuals who have had a history of cancer, but have been responsive to another mode of therapy. The prior therapy may have included, but is not restricted to, surgical resection, radiotherapy, and traditional chemotherapy. As a result, these individuals have no clinically measurable tumor. However, they are suspected of being at risk for progression of the disease, either near the original tumor site, or by metastases.

This group can be further subdivided into high-risk and low-risk individuals. The subdivision is made on the basis of features observed before or after the initial treatment. These features are known in the clinical arts, and are suitably defined for each different cancer. Features typical of high risk subgroups are those in which the tumor has invaded neighboring tissues, or who show involvement of lymph nodes.

Another suitable group of subjects is those with a genetic predisposition to cancer but who have not yet evidenced clinical signs of cancer. For instance, women with a family history of breast cancer, but still of childbearing age, may avail themselves of having their breast tissue examined for expression levels of Notch1 and those testing positive, e.g., having higher than normal expression level of Notch1 may wish to be monitored for presenting with breast cancer or alternatively avail themselves of preventive treatment with a conventional Notch1 specific monoclonal therapy.

A variety of other immunoassays are available for detecting Notch1. For example, by labeling the antibodies, or binding fragments thereof, with a radioisotope, a radioimmunoassay (RIA) can be used to detect cancer-specific antigens (e.g., Current Protocols in Immunology, Volumes 1 and 2, Coligen et al., Ed. Wiley-Interscience, New York, N.Y., Pubs. (19910, Colcher et al., 1981, Cancer Research, 41, 1451 1459; Weintraub, “Principles of Radioimmunoassays”, Seventh Training Course on Radioligand Techniques, The Endocrine Society, March, 1986). The radioactive isotope label can be detected by using a gamma counter or a scintillation counter or by radiography. Representative radioisotopes include ³⁵S, ¹⁴C, ¹²⁵I, ³H, and ¹³¹I. Procedures for labeling biological agents with the radioactive isotopes are generally known in the art. Tritium labeling procedures are described in U.S. Pat. No. 4,302,438, which is hereby incorporated by reference. Iodinating, tritium labeling, and ³⁵S labeling procedures especially adapted for murine monoclonal antibodies are well known. Other procedures for iodinating biological agents, such as antibodies, binding portions thereof, probes, or ligands, are described by Hunter and Greenwood, Nature 144:945 (1962), David et al., Biochemistry 13:1014-1021 (1974), and U.S. Pat. Nos. 3,867,517 and 4,376,110, which are hereby incorporated by reference. Procedures for iodinating biological agents are described by Greenwood, F. et al., Biochem. J. 89:114-123 (1963); Marchalonis, J., Biochem. J. 113:299-305 (1969); and Morrison, M. et al., Immunochemistry, 289-297 (1971), which are hereby incorporated by reference. Procedures for ⁹⁹mTc-labeling are described by Rhodes, B. et al. in Burchiel, S. et al. (eds.), Tumor Imaging: The Radioimmunochemical Detection of Cancer, New York: Masson 111-123 (1982) and the references cited therein, which are hereby incorporated by reference. Procedures suitable for “In-labeling biological agents are described by Hnatowich, D. J. et al., J. Immul. Methods, 65:147-157 (1983), Hnatowich, D. et al., J. Applied Radiation, 35:554-557 (1984), and Buckley, R. G. et al., F.E.B.S. 166:202-204 (1984), which are hereby incorporated by reference.

The presently universally-accepted method for the diagnosis of solid cancer is the histologic determination of abnormal cellular morphology in surgically biopsied or resected tissue. Once removed, the tissue is preserved in a fixative, embedded in paraffin wax, cut into 5 μm-thick sections, and stained with two dyes: hematoxylin for the nucleus and eosin for the cytoplasm (”H&E staining“). This approach is simple, fast, reliable, and inexpensive. Histopathology allows the diagnosis of a variety of tissue and cell types. By providing an estimation of tumor “Grade” (cellular differentiation/tissue architecture) and “Stage” (depth of organ penetration) it also makes prognosis possible.

Immunohistochemistry (“IHC”) techniques utilize an antibody to probe and visualize cellular antigens in situ, generally by chromogenic or fluorescent methods. For sample preparation, a tissue or cell sample from a mammal (typically a human patient) may be used. Examples of samples include, but are not limited to, cancer cells such as colon, breast, prostate, ovary, lung, stomach, pancreas, lymphoma, and leukemia cancer cells. The sample can be obtained by a variety of procedures known in the art including, but not limited to surgical excision, aspiration or biopsy. The tissue may be fresh or frozen. In one embodiment, the sample is fixed and embedded in paraffin or the like. The tissue sample may be fixed (i.e. preserved) by conventional methodology. One of ordinary skill in the art will appreciate that the choice of a fixative is determined by the purpose for which the sample is to be histologically stained or otherwise analyzed. One of ordinary skill in the art will also appreciate that the length of fixation depends upon the size of the tissue sample and the fixative used.

IHC may be performed in combination with additional techniques such as morphological staining and/or fluorescence in-situ hybridization. Two general methods of IHC are available; direct and indirect assays. According to the first assay, binding of antibody to the target antigen (e.g., Notch1) is determined directly. This direct assay uses a labeled reagent, such as a fluorescent tag or an enzyme-labeled primary antibody, which can be visualized without further antibody interaction. In a typical indirect assay, unconjugated primary antibody binds to the antigen and then a labeled secondary antibody binds to the primary antibody. Where the secondary antibody is conjugated to an enzymatic label, a chromogenic or fluorogenic substrate is added to provide visualization of the antigen. Signal amplification occurs because several secondary antibodies may react with different epitopes on the primary antibody.

The primary and/or secondary antibody used for immunohistochemistry typically will be labeled with a detectable moiety. Numerous labels are available, some of which are detailed herein.

Aside from the sample preparation procedures discussed above, further treatment of the tissue section prior to, during or following IHC may be desired, for example, epitope retrieval methods, such as heating the tissue sample in citrate buffer may be carried out (see, e.g., Leong et al. Appl. Immunohistochem. 4(3):201 (1996)).

Following an optional blocking step, the tissue section is exposed to primary antibody for a sufficient period of time and under suitable conditions such that the primary antibody binds to the target protein antigen in the tissue sample. Appropriate conditions for achieving this can be determined by routine experimentation. The extent of binding of antibody to the sample is determined by using any one of the detectable labels discussed above. Preferably, the label is an enzymatic label (e.g. HRPO) which catalyzes a chemical alteration of the chromogenic substrate such as 3,3′-diaminobenzidine chromogen. Preferably the enzymatic label is conjugated to antibody which binds specifically to the primary antibody (e.g. the primary antibody is rabbit polyclonal antibody and secondary antibody is goat anti-rabbit antibody).

Specimens thus prepared may be mounted and coverslipped. Slide evaluation is then determined, e.g. using a microscope.

Alternatively, one may also utilize microscope-based cell imaging, which uses conventional light microscopy combined with monochromatic light filters and computer software programs. The wavelengths of the light filters are matched to the colors of the antibody stain and the cell counterstain. The filters allow the microscopist to identify, classify and then measure differences in the optical density of specific colors of light transmitted through immunostained portions of tissue sections. See U.S. Pat. Nos. 5,235,522 and 5,252,487, both of which are incorporated herein by reference, for applications of these methods to tumor protein measurement. Yet other cell imaging systems (image cytometers) permit automated recognition of features, and combine this with automated calculation of feature areas, automated calibration, and automatic calculation of average and integrated (SOD) optical density. (See, e.g., U.S. Pat. Nos. 5,548,661, 5,787,189, both of which are incorporated herein by reference, and references therein.)

Since immunohistochemical staining of tissue sections has been shown to be a reliable method of assessing or detecting presence of proteins in a sample. Consequently, use of the antibodies described herein to score staining and/or detection levels are also contemplated.

Protein expression may be determined using a validated scoring method (Dhanasekaran et al., 2001, Nature 412, 822-826; Rubin et al., 2002, supra; Varambally et al., 2002, Nature 419, 624-629) where staining was evaluated for intensity and the percentage of cells staining positive. In cases where benign tissue and cancer are present, only one or the other tissue type is evaluated for purposes of analysis. Any of the methods of the invention may score the analysis by using a scale of 0 to 4, where 0 is negative (no detectable Notch or level of expression same as that of a control sample) and 4 is high intensity staining in the majority of cells. In certain embodiments, the scoring may be used for diagnostic or prognostic purposes. For example, a score of 1, while a positive score, may indicate better prognosis than, say, a score of 3 or 4.

The information gathered in accordance with the invention will aid the physician in determining a course of treatment for a patient presenting with a Notch-mediated oncogenic disorder. For example, in the case of tumor cells comprising Notch receptor expressing, a low score might dictate that additional intervention, e.g., surgery is not warranted. Typically, a staining pattern score of about 3+ or higher in an IHC assay is diagnostic and/or prognostic. In some embodiments, a staining pattern score of about 1+ or higher is diagnostic and/or prognostic. In other embodiments, a staining pattern score of about 2+ or higher is diagnostic and/or prognostic. It is understood that when cells and/or tissue from a tumor are examined using IHC, staining is generally determined or assessed in tumor cell and/or tissue (as opposed to stromal or surrounding tissue that may be present in the sample).

Other assay methods, known as competitive or sandwich assays, are well established and widely used in the commercial diagnostics industry.

Competitive assays rely on the ability of a tracer Notch1 analogue to compete with the test sample Notch1 for a limited number of anti-Notch1 antibody antigen-binding sites. The anti-Notch1 antibody generally is insolubilized before or after the competition and then the tracer and Notch1 bound to the anti-Notch1 antibody are separated from the unbound tracer and Notch1. This separation is accomplished by decanting (where the binding partner was preinsolubilized) or by centrifuging (where the binding partner was precipitated after the competitive reaction). The amount of test sample Notch1 is inversely proportional to the amount of bound tracer as measured by the amount of marker substance. Dose-response curves with known amounts of Notch1 are prepared and compared with the test results to quantitatively determine the amount of Notch1 present in the test sample. These assays are called ELISA systems when enzymes are used as the detectable markers.

Another species of competitive assay, called a “homogeneous” assay, does not require a phase separation. Here, a conjugate of an enzyme with the Notch1 is prepared and used such that when anti-Notch1 antibody binds to the Notch1 the presence of the anti-Notch1 antibody modifies the enzyme activity. In this case, the Notch1 or its immunologically active fragments are conjugated with a bifunctional organic bridge to an enzyme such as peroxidase. Conjugates are selected for use with anti-Notch1 antibody so that binding of the anti-Notch1 antibody inhibits or potentiates the enzyme activity of the label. This method per se is widely practiced under the name of EMIT.

Steric conjugates are used in steric hindrance methods for homogeneous assay. These conjugates are synthesized by covalently linking a low-molecular-weight hapten to a small Notch1 fragment so that antibody to hapten is substantially unable to bind the conjugate at the same time as anti-Notch1 antibody. Under this assay procedure the Notch1 present in the test sample will bind anti-Notch1 antibody, thereby allowing anti-hapten to bind the conjugate, resulting in a change in the character of the conjugate hapten, e.g., a change in fluorescence when the hapten is a fluorophore.

Sandwich assays particularly are useful for the determination of Notch1 or anti-Notch1 antibodies. In sequential sandwich assays an immobilized anti-Notch1 antibody is used to adsorb test sample Notch1, the test sample is removed as by washing, the bound Notch1 is used to adsorb a second, labeled anti-Notch1 antibody and bound material is then separated from residual tracer. The amount of bound tracer is directly proportional to test sample Notch1. In “simultaneous” sandwich assays the test sample is not separated before adding the labeled anti-Notch1. A sequential sandwich assay using an anti-Notch1 monoclonal antibody as one antibody and a polyclonal anti-Notch1 antibody as the other is useful in testing samples for Notch1.

In another embodiment, the invention provides a method for determining the effect of a therapeutic regimen for alleviating a Notch1 mediated disorder, wherein the regimen comprises the use of an Notch1 antagonist or an agonist antibody, the method comprising the steps of: a) obtaining a cell or tissue sample from an individual undergoing the therapeutic regimen b) measuring the levels of Notch1 in the cell or tissue sample; c) scoring the sample for Notch1 protein levels, and d) comparing the levels to that of a control sample to predict the responsiveness of the Notch1 mediated disorder to the therapeutic regimen. Thus, a low score, e.g., 0 or a lowering score over time suggests that the treatment regimen comprising a Notch1 antagonist, e.g., Notch1 specific antibody, is effective in reducing tumor burden or Notch1 expressing cells or level of Notch1 expression.

In certain embodiments, the methods of the invention propose contacting the sample of interest with an antibody to Notch1. In certain embodiments, the detecting is done on histological or tissue sections or cytological preparations by immunohistochemistry or immunocytochemistry. As well, detecting Notch1 may be done by immunoblotting or by Fluorescence-Activated Cell Sorting (FACS).

The invention is also directed to a method for predicting disease-free survival and/or overall survival in a patient diagnosed with an oncogenic disorder associated with Notch1 expression comprising: a) obtaining a sample of diseased or cancerous tissue from an individual presenting with an oncogenic disorder, b) detecting levels of Notch1 expressing cells in the cancer cells or cancer tissue of the sample, c) scoring the samples for expression of Notch1 levels; and d) comparing the scoring to that obtained from a control sample to determine likelihood of disease-free survival and overall survival associated with Notch1. Preferably, the scoring comprises using a scale of 0 to 4, where 0 is negative (no detectable Notch1 or level of Notch1 comparable to a control level), and 4 is high intensity staining in the majority of cells and wherein a score of 1 to 4 (i.e. a positive score) indicates a poor prognosis for disease free and overall survival in patients with said disorder.

A method for screening for metastatic potential of solid tumors is also provided. The method comprises a) obtaining a sample of tumor tissue from an individual in need of screening for metastatic potential of a solid tumor; b) reacting an antibody to Notch1 with tumor tissue from the patient; c) detecting the extent of binding of the antibody to the tissue and d) correlating the extent of binding of the antibody with its metastatic potential.

The present invention further encompasses in vivo imaging methods useful for visualizing the presence of Notch1 expressing cells indicative of an oncogenic disorder. Such techniques allow for a diagnosis without the use of an unpleasant biopsy or other invasive diagnostic technique. The concentration of detectably labeled anti-Notch1 antibody of the invention which is administered should be sufficient such that the binding to those cells having or expressing the Notch1 antigen is detectable compared to the background. Further, it is desirable that the detectably labeled anti-Notch1 antibody of the invention be rapidly cleared from the circulatory system in order to give the best target-to-background signal ratio.

Imaging analysis is well known in the medical art, and includes, without limitation, x-ray analysis, magnetic resonance imaging (MRI) or computed tomography (CE). As indicated supra, preferably, the Notch1 antibodies used in the in vivo (and also in vitro) diagnostic methods are directly or indirectly labeled with a detectable substance/label that can be imaged in a patient. Suitable detectable substances include various enzymes, prosthetic groups, fluorescent materials, luminescent materials and radioactive materials. As a rule, the dosage of detectably labeled anti-Notch1 antibody of the invention for in vivo diagnosis is somewhat patient-specific and depends on such factors as age, sex, and extent of disease. Dosages may also vary, for example, depending on number of injections given, tumor burden, and other factors known to those of skill in the art. For instance, tumors have been labeled in vivo using cyanine-conjugated Mabs. Ballou et al. (1995) Cancer Immunol. Immunother. 41:257 263.

In the case of a radiolabeled biological agent, the biological agent is administered to the patient and is localized to the tumor bearing the antigen (Notch1 receptor protein) with which the biological agent reacts, and is detected or “imaged” in vivo using known techniques such as radionuclear scanning using e.g., a gamma camera or emission tomography. See e.g., A. R. Bradwell et al., “Developments in Antibody Imaging”, Monoclonal Antibodies for Cancer Detection and Therapy, R. W. Baldwin et al., (eds.), pp. 65-85 (Academic Press 1985), which is hereby incorporated by reference. Alternatively, a positron emission transaxial tomography scanner, such as designated Pet VI located at Brookhaven National Laboratory, can be used where the radiolabel emits positrons (e.g., ¹¹C, ¹⁸F, ¹⁵O, and ¹³N).

In one embodiment the invention provides for the use of the Notch1 antibodies in the diagnosis of cancer, by specifically allowing one to detect and visualize tissues that express Notch1 or contain Notch1 expressing cells (e.g., cancer). The method comprises: (i) administering to a subject (and optionally a control subject) a diagnostically effective amount of detectably labeled anti-Notch1 antibody of the invention or an antigen-binding fragment thereof or a pharmaceutical composition thereof comprising as an active component the antibodies of the invention or binding fragments thereof that specifically bind Notch1, under conditions that allow interaction of the antibodies to Notch1 to occur; and (ii) detecting the binding agent, for example, to locate Notch1 expressing tissues or otherwise identify Notch1 expressing cells. The term “diagnostically effective” means that the amount of detectably labeled anti-Notch1 antibody of the invention is administered in sufficient quantity to enable detection of neoplasia.

In certain embodiments, the antibodies of the invention may be labeled with a contrast agent, such as barium, which can be used for x-ray analysis, or a magnetic contrast agent, such as a gadolinium chelate, which can be used for MRI or CE.

In another embodiment of the method, a biopsy is obtained from the patient to determine whether the tissue of interest expresses Notch1 rather than subjecting the patient to imaging analysis.

A radiolabeled antibody or immunoconjugate may comprise a gamma.-emitting radioisotope or a positron-emitter useful for diagnostic imaging. The label used will depend on the imaging modality chosen. The use of antibodies for in vivo diagnosis is well known in the art. Sumerdon et al., (Nucl. Med. Biol 17:247-254 (1990)) have described an optimized antibody-chelator for the radioimmunoscintographic imaging of tumors using Indium-111 as the label. Griffin et al., (J Clin Onc 9:631-640 [1991]) have described the use of this agent in detecting tumors in patients suspected of having recurrent colorectal cancer.

The methods of the present invention may also use paramagnetic isotopes for purposes of in vivo detection. The use of similar agents with paramagnetic ions as labels for magnetic resonance imaging is also known in the art -Lauffer, Magnetic Resonance in Medicine 22:339-342 (1991).

Radioactive labels such as Indium-111, Technetium-99m, or Iodine-131 can be used for planar scans or single photon emission computed tomography (SPECT). Positron emitting labels such as Fluorine-19 can also be used for positron emission tomography (PET). For MRI, paramagnetic ions such as Gadolinium (III) or Manganese (II) can be used.

For in vivo diagnostic imaging, the type of detection instrument available is a major factor in selecting a given radioisotope. The radioisotope chosen must have a type of decay which is detectable for a given type of instrument. Still another important factor in selecting a radioisotope for in vivo diagnosis is that the half-life of the radioisotope be long enough so that it is still detectable at the time of maximum uptake by the target, but short enough so that deleterious radiation with respect to the individual is minimized. Ideally, a radioisotope used for in vivo imaging lacks a particle emission, but produces a large number of photons in the 140 250 keV range, to be readily detected by conventional gamma cameras.

Radioactive metals with half-lives ranging from 1 hour to 3.5 days are available for conjugation to antibodies, such as scandium-47 (3.5 days) gallium-67 (2.8 days), gallium-68 (68 minutes), technetiium-99m (6 hours), and indium-111 (3.2 days), of which gallium-67, technetium-99m, and indium-111 are preferable for gamma camera imaging, gallium-68 is preferable for positron emission tomography. Labels such as Indium-111, Technetium-99m, or Iodine-131 can be used for planar scans or single photon emission computed tomography (SPECT).

In the case of the radiometals conjugated to the specific antibody, it is likewise desirable to introduce as high a proportion of the radiolabel as possible into the antibody molecule without destroying its immunospecificity. A further improvement may be achieved by effecting radiolabeling in the presence of the specific cancer marker of the present invention, to insure that the antigen binding site on the antibody will be protected. The antigen is separated after labeling.

Suitable radioisotopes, particularly in the energy range of 60 to 4,000 keV, include, ⁵¹Cr, ⁵⁷Co, ⁵⁸Co, ⁵⁹Fe, 131I, 121I, .124I, 86Y, 62Cu, 64Cu, 111In, 67Ga, 68Ga, 99mTc, 94mTc, 18F, 11C, 13N, 15O, 75Br, 75Se, 97Ru, 99mTc, 111In, 114mIn, 123I, 125I, 131I, 169Yb, 197Hg, and 201T1, and the like. See for example, U.S. Patent Application entitled “Labeling Targeting Agents with Gallium-68”—Inventors G. L. Griffiths and W. J. McBride, (U.S. Provisional Application No. 60/342,104), which discloses positron emitters, such as 18F, .68Ga, 94mTc. and the like, for imaging purposes and which is incorporated in its entirety by reference. Particularly useful diagnostic/detection radionuclides include, but are not limited to, 18F, 52Fe, 62Cu, 64Cu, .67Cu, 67Ga, 68Ga, .86Y, 89Zr, 94mTc, 94mTc, .99mTc, .111In, 123I, 124I, 125I, .131I, 154-158Gd, 32P, 90Y, 188Re, and 175Lu.

Decay energies of useful gamma-ray emitting radionuclides are preferably 20 2000 keV, more preferably 60 600 keV, and most preferably 100 300 keV.

Radionuclides useful for positron emission tomography include, but are not limited to: 18F, 1Mn, 2mMn, 52Fe, 55Co, 62Cu, 64Cu, 68Ga, 72As, 75Br, 76Br, 82mRb, 83Sr, 86Y, 89Zr, 94mTc, 110In, 120I, and 124I. Total decay energies of useful positron-emitting radionuclides are preferably <2,000 keV, more preferably under 1,000 keV, and most preferably <700 keV.

Also contemplated by the present invention is the use of non-radioactive agents as diagnostic reagents. A suitable non-radioactive diagnostic agent is a contrast agent suitable for magnetic resonance imaging, computed tomography or ultrasound. Magnetic imaging agents include, for example, non-radioactive metals, such as manganese, iron and gadolinium, complexed with metal-chelate combinations that include 2-benzyl-DTPA and its monomethyl and cyclohexyl analogs, when used along with the antibodies of the invention. See U.S. Ser. No. 09/921,290 filed on Oct. 10, 2001, which is incorporated in its entirety by reference.

Bispecific antibodies are also useful in targeting methods and provide a preferred way to deliver two diagnostic agents to a subject. U.S. Ser. Nos. 09/362,186 and 09/337,756 discloses a method of pretargeting using a bispecific antibody, in which the bispecific antibody is labeled with ²⁵¹I and delivered to a subject, followed by a divalent peptide labeled with ⁹⁹mTc and are incorporated herein by reference in their entirety. Pretargeting methods are also described in U.S. Pat. No. 6,962,702 (Hansen et al.), U.S. Ser. No. 10/150,654 (Goldenberg et al.), and Ser. No. 10/768,707 (McBride et al.), which are all also incorporated herein by reference in their entirety. The delivery results in excellent tumor/normal tissue ratios for ¹²⁵1 and ⁹⁹mTc, thus showing the utility of two diagnostic radioisotopes. Any combination of known diagnostic agents can be used to label the antibodies. The binding specificity of the antibody component of the MAb conjugate, the efficacy of the therapeutic agent or diagnostic agent and the effector activity of the Fc portion of the antibody can be determined by standard testing of the conjugates.

A diagnostic agent can be attached at the hinge region of a reduced antibody component via disulfide bond formation. As an alternative, such peptides can be attached to the antibody component using a heterobifunctional cross-linker, such as N-succinyl 3-(2-pyridyldithio)propionate (SPDP). Yu et al., Int. J. Cancer 56: 244 (1994). General techniques for such conjugation are well-known in the art. See, for example, Wong, CHEMISTRY OF PROTEIN CONJUGATION AND CROSS-LINKING (CRC Press 1991); Upeslacis et al., “Modification of Antibodies by Chemical Methods,” in MONOCLONAL ANTIBODIES: PRINCIPLES AND APPLICATIONS, Birch et al. (eds.), pages 187 230 (Wiley-Liss, Inc. 1995); Price, “Production and Characterization of Synthetic Peptide-Derived Antibodies,” in MONOCLONAL ANTIBODIES: PRODUCTION, ENGINEERING AND CLINICAL APPLICATION, Ritter et al. (eds.), pages 60 84 (Cambridge University Press 1995).

Methods for conjugating peptides to antibody components via an antibody carbohydrate moiety are also well-known to those of skill in the art. See, for example, Shih et al., Int. J. Cancer 41: 832 (1988); Shih et al., Int. J. Cancer 46: 1101 (1990); and Shih et al., U.S. Pat. No. 5,057,313, all of which are incorporated in their entirety by reference. The general method involves reacting an antibody component having an oxidized carbohydrate portion with a carrier polymer that has at least one free amine function and that is loaded with a plurality of peptide. This reaction results in an initial Schiff base (imine) linkage, which can be stabilized by reduction to a secondary amine to form the final conjugate.

The Fc region is absent if the antibody used as the antibody component of the immunoconjugate is an antibody fragment. However, it is possible to introduce a carbohydrate moiety into the light chain variable region of a full length antibody or antibody fragment. See, for example, Leung et al., J. Immunol. 154: 5919 (1995); Hansen et al., U.S. Pat. No. 5,443,953 (1995), Leung et al, U.S. Pat. No. 6,254,868, all of which are incorporated in their entirety by reference. The engineered carbohydrate moiety is used to attach the therapeutic or diagnostic agent.

In situ detection can be accomplished by removing a histological specimen from a patient, and providing the combination of labeled antibodies of the present invention to such a specimen. The antibody (or fragment) is preferably provided by applying or by overlaying the labeled antibody (or fragment) to a biological sample. Through the use of such a procedure, it is possible to determine not only the presence of Notch1 but also the distribution of Notch1 in the examined tissue. Using the present invention, those of ordinary skill will readily perceive that any of a wide variety of histological methods (such as staining procedures) can be modified in order to achieve such in situ detection.

Also provided by the invention is in vivo biophotonic imaging (Xenogen, Almeda, Calif.) which utilizes real-time luciferase. The luciferase gene is incorporated into cells, microorganisms, and animals (e.g., as a fusion protein with a marker of the present invention). When active, it leads to a reaction that emits light. A CCD camera and software is used to capture the image and analyze it.

In another embodiment, the anti-Notch1 antibody is unlabeled and imaged by administering a second antibody or other molecule that is detectable and that can bind the anti-Notch1 antibody. A specifically bound and labeled antibody can be detected in the patient using known methods, including, but not limited to, radionuclide imaging, positron emission tomography, computerized axial tomography, X-ray or magnetic resonance imaging method, fluorescence detection, and chemiluminescent detection.

In vivo imaging methods can also be used for developing a prognostic evaluation of the condition of a patient suspected of exhibiting an oncogenic disorder mediated by Notch1.

### Purification

Still further, the anti-Notch1 antibodies described herein may also be used as affinity purification agents. In this process, the antibodies are immobilized on a solid phase such a Sephadex resin or filter paper, using methods well known in the art. The immobilized antibody is contacted with a sample containing the Notch1 protein (or fragment thereof) to be purified, and thereafter the support is washed with a suitable solvent that will remove substantially all the material in the sample except the Notch1 protein, which is bound to the immobilized antibody. Finally, the support is washed with another suitable solvent, such as glycine buffer, pH 5.0, that will release the Notch1 protein from the antibody.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, etc. The containers may be formed from a variety of materials such as glass or plastic. The container holds a composition which is by itself or when combined with another composition(s) effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice, such as cancer. Moreover, the article of manufacture may comprise (a) a first container with a composition contained therein, wherein the composition comprises an antibody of the invention; and (b) a second container with a composition contained therein, wherein the composition comprises a further therapeutic agent, including, e.g. a chemotherapeutic agent or an anti-angiogenesis agent, including, e.g., an anti-VEGF antibody (e.g. bevacizumab). The article of manufacture in this embodiment of the invention may further comprise a package insert indicating that the first and second antibody compositions can be used to treat a particular condition, e.g. cancer. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for Notch receptor cell killing assays, for purification or immunoprecipitation of Notch receptor from cells. For isolation and purification of Notch receptor, the kit can contain an anti-Notch receptor antibody coupled to beads (e.g., sepharose beads). Kits can be provided which contain the antibodies for detection and quantitation of Notch receptor in vitro, e.g. in an ELISA or a Western blot. As with the article of manufacture, the kit comprises a container and a label or package insert on or associated with the container. The container holds a composition comprising at least one anti-Notch receptor antibody of the invention. Additional containers may be included that contain, e.g., diluents and buffers, control antibodies. The label or package insert may provide a description of the composition as well as instructions for the intended in vitro or diagnostic use.

The foregoing are merely exemplary detection assays for Notch1. Other methods now or hereafter developed that use anti-Notch1 antibody for the determination of Notch1 are included within the scope hereof, including the bioassays described herein.

The following are examples of the methods and compositions of the invention. It is understood that various other embodiments may be practiced, given the general description provided above.

### EXAMPLES

### Example 1

### Generation of 293 Cell Line Stably Expressing Human Notch1, 2, 3 and 4

Gene synthesis and Vector Construction for human full-length Notch1, 2, 3 and 4 with FLAG tag at its C-terminus. Stable cell lines were generated for all four Notch genes (Notch1, Notch2, Notch3 and Notch4) with FLAG tag using the same strategy, vector and cell substrate. Only Notch1 is described as a representative in this example. The full-length coding region, along with their respective 5′- and 3′-flanking sequences for Notch1, was chemically synthesized at DNA2.0. The open reading frame was tagged with FLAG peptide (DYKDDDDK) at the C-terminal end before the stop codon (Appendix I and II). The flanking sequences contain cloning site HindIll and GCCGCCACC in front of the ATG start codon at its 5′-end and stop codon followed by cloning site NotI at its 3′-end. Plasmids containing the HindIII-NotI DNA fragments were received from DNA2.0 and the HindIII-NotI fragments were isolated. The vector pcDNA5/FRT/T0 (Invitrogen) was used to express the FLAG tagged full-length Notch1, open reading frame. The pcDNA5/FRT/T0 contains two features, together with matching cell lines, needed for the generation of Notch stable cell lines. For one, the vector pcDNA5/FRT/T0 allows the expression of Notch1 with FLAG tag. The vector contains human cytomegalovirus early promoter with two tetracycline operators to allow tight control of Notch1 expression in cell lines where the tetracycline repressor is expressed. For the other, the vector pcDNA5/FRT/T0 contains the FRT sequence in front of promoter-less and ATG-less hygromycin gene, which allows the homologous recombination between the vector FRT sequence and the identical chromosome FRT sequence in the presence of the FLIP recombinase provided by co-transfection of pOG44 vector (Invitrogen). The matching cell line 293 TREX FLIP contains both tetracycline repressor allowing tight control of Notch1 expression and FRT sequence permitting homologous recombination. The homologous recombination between the vector FRT sequence and host cell FRT sequence will allow the FLIP-IN of the entire plasmid sequence and hence the Notch1 expression cassette. At the same time, the FRT sequence in the host cell chromosome was designed such that it can put the promoter-less and ATG-less hygromycin gene in frame with the start codon ATG to confer the transfected cells with expected homologous recombination hygromycin resistant. The vector pcDNA5/FRT/T0 was digested with HindIII+NotI and the Notch fragment digested with the same set of enzymes was ligated with the linearized vector to form pcDNA5/FRT/T0-HN1+FLAG. Ligation product was transformed into XL2 Blue MRF′ cells and transformants were screened and positive clones sequencing confirmed.

Generation of 293 cell line stably expressing human full-length Notch1. Inducible expression stable cell lines were generated using Invitrogen FLPIn system by transfecting pcDNA5/FRT/T0-HN1+FLAG into 293 FLPIn TREX. The description and the rationale of the cell line are provided in the earlier section. To generate stable cell lines, pOG44, which provides constitutively expressed FLP recombinase (flp-F70L), and the pcDNA5/FRT based vector, pcDNA5/FRT/T0-HN1+FLAG were co-transfected into the 293 FLPIn/TREX host cell line. FLP recombinase mediates FRT site specific insertion of the construct by homologous recombination between FRT sites in the expression vector and those integrated into the 293 FLPIn TREX cell genome. After FRT insertion hygromycin resistance is used as a selection marker. pOG44 was co-transfected with pcDNA5/FRT/T0-HN1+FLAG in a 9:1 ratio (w/w) pOG44:expression plasmid using Fugene6 (Roche) lipid-based transfection reagent in a 4:1 (v/w) ratio. Cells are re-fed 48 hours post transfection with media containing hygromycin (100 ug/ml) selection agent. Colonies are expanded to generate stable expression cell lines.

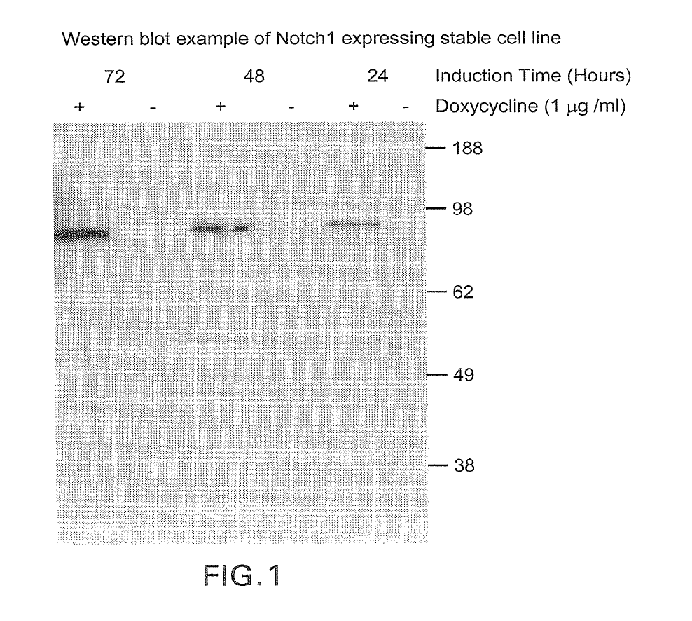

Stable Cell Line Characterization. Stable cell line expression screening was completed with Western blot analysis (FIG. 1). Cells were lysed with 2× lysis buffer+Protease inhibitor Cocktail and loaded to 4-12% Bis-Tris gel and run under denaturing and reducing conditions. Proteins were transferred to PVDF membrane and blocked overnight in 12% milk. Detection was completed using a 1:2000 dilution of Anti-Flag M2 peroxidase conjugated antibody (Sigma) incubated at RT for 120 minutes. Kinetic expression of stably expressed human full-length Notch1 as functions of time and tetracycline concentration, respectively, was conducted where optimal dose/time effect for the Notch lines was identified.

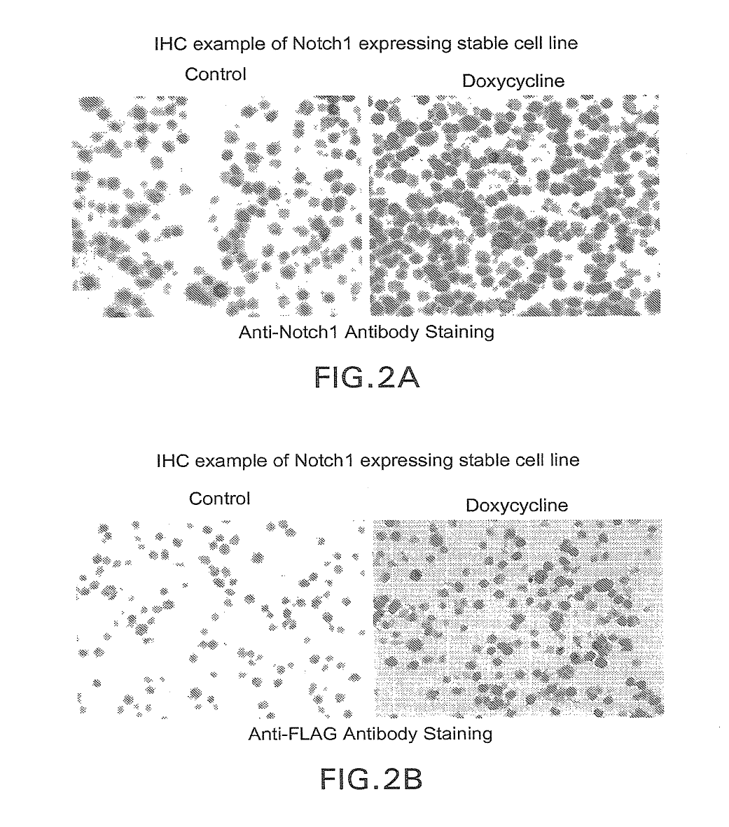

Immunohistochemical (IHC) staining of stable cell lines. The human full-length Notch1 expressing stable cell lines were analyzed by IHC. Confluent T225 flasks were trypsinized (HyClone, SH30236-01) and the resulting cell suspension was pelleted by centrifugation at 250× g for 10 minutes. The media was aspirated and 3-4 drops of warmed Histogel (Richard-Allan Scientific HG-4000-012) was added to each loosened cell pellet. The Histogel pellet was mixed and cooled at 2-8° C. for 60 minutes and placed in 10% formalin for 16-24 hours. The Histogel pellet was infiltrated with 70% ethanol, 95% ethanol, 100% ethanol, xylenes, and paraffin overnight (Sakura VIP5A-F1). The Histogel pellet was then embedded in paraffin (Sakura TEC5EMA-15101), cut at 5 μm, and mounted onto Superfrost plus slides. Sections were deparaffinized, rehydrated, and placed in Target Retrieval Buffer 1× (Dako S1699) in a Decloaking Chamber (Biocare Medical DC2002) for heat-induced epitope retrieval at 125° C. for 30 seconds. Endogenous peroxidase activity was blocked using Peroxidase Blocking Reagent (Dako K4007) for ten minutes. Sections were washed with Phosphate Buffered Saline (PBS), and incubated with anti-Notch 1 (0.02 μg/ml, Santa Cruz, SC-6014R), anti-Notch 2, (1.6 μg/ml, Santa Cruz, SC-5545), anti-Notch 3 (0.2 μg/ml, Abcam, ab23426), anti-Notch 4 (0.5 μg/ml, Santa Cruz, SC-5594) and anti-FLAG M2 mouse monoclonal antibody Peroxidase Conjugate (1:100, Sigma, A8592) for 30 minutes at room temperature. Sections were washed with PBS and incubated with Envision+polymer (no polymer used for anti-FLAG M2) for 30 minutes at room temperature, washed with PBS, and diaminobenzidine was used for development of a brown reaction product (Dako K4007). The slides were immersed in hematoxylin for 30 seconds to counterstain (Sigma MHS32). The staining was visualized and shown in FIG. 2.

### Example 2

### Solid Phase Panning Procedures

Phage antibody display libraries in single chain (ScFv) format were derived from a phage library. Three libraries including BMV (bone marrow), CS (spleen) and DP47 (frame work based CS) were used for panning Notch 1 binding antibodies. Human IgG Fc protein was purchased from Chemicon International (cat#AG714) and was used for de-selection in panning Notch1 extracellular fragment (amino acid 19-526) with Fc fusion protein was purchased from R&D Systems (catalog number: 3647-TK). Helper phages (M13K07^{trp}) used for rescuing phagemid and phage amplification were provided by CAT.

Elution buffer preparation was done as following: add acetic acid to high quality deionized water to a final concentration of 50 mM and filter through 0.22 um filter to sterilize solution. Trypsin (Sigma cat#T-1426, from bovine pancreas) was dissolved in 50 mM acetic acid to give a 500× stock solution (5 mg/ml) and small volume aliquots were stored at −20° C. Working solution (1×) was made freshly each time by diluting 500× stock solution into 0.1 M sodium phosphate buffer at pH 7.0.

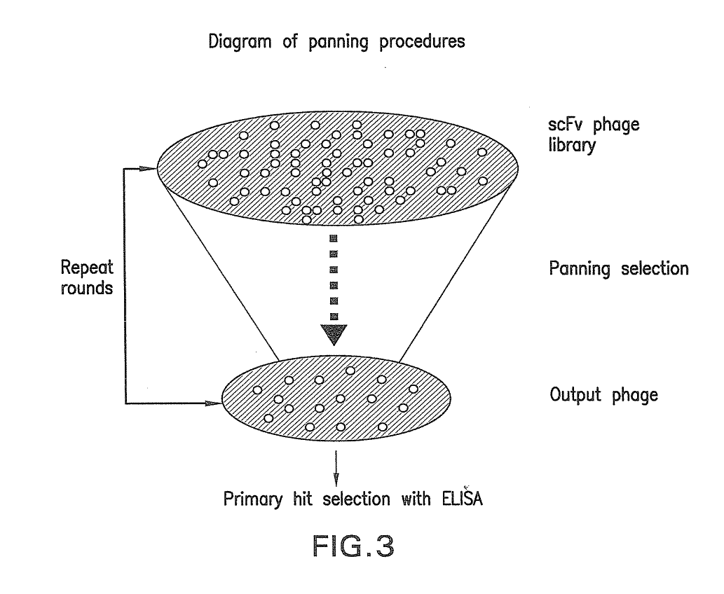

Panning procedures are outlined in FIG. 3. The first step is coating Notch1 antigen on Maxisorp 96-well plate at 25 ug/ml concentration in Dulbecco's PBS (GIBCO) over night at 4° C. Panning round 1 used 4 wells and 75 ug of Notch1 was used in each well. After coating, the wells with coated Notch antigen were rinsed 3 times with 1× PBS to remove the unbound antigen, then blocked with 5% (w/v) skimmed milk in PBS-D for 2 hours at room temperature. The library was supplied in 50 ul aliquots, each 50 ul contains 10̂12 phage particles. For the first round panning, 50 ul of the phage library was mixed with 950 ul of 5% milk blocker in PBS with 0.1% Tween in a 1.6 ml tube, and human Fc protein (200 ug/ml) was also added and incubated at RT for 1 hour to block phage and neutralize Fc binding. After pre-blocking step, the blocked phage library was incubated with Notch1 antigen in the coated well with slow mixing at RT for 2 hours. Following washing with PBS-Tween and PBS, 250 ul per well of Trypsin solution was added and incubated at 37° C. incubator with cover for 30 min to elute bound phages.

To recover eluted phages, E. coli (TG1) cells were grown from a single colony in a flask with 50 ml of 2-TY medium at 37° C. with shaking 300 rpm until cell density reaching OD600nm at 0.5-0.8. The prepared TG1 cells were added to the eluted phage solution and incubated at 37° C. for 1 hour with slowing shaking at 100 rpm. After the infection step, Tg1 cells were plated on selection agar plate and incubated at 30° C. over night. Single colonies and pool of colonies were used for phage preparation to be used in next round panning or ELISA screening.

Phage can be rescued and amplified with E. coli using helper phage. MK13K07 helper phage engineered with trypsin cleavage site was added to E. coli cells at growth phase (OD600 at 0.5-0.8) at ratio of 10 helper phage per cell and incubated at 37° C. for 30 minutes without mixing, then at 150 RPM for an additional 30 minutes for helper phage infection. Then, media with helper phage solution were removed by spinning down cells at 3000 g×10 minutes. After removing all supernatants, cells were re-suspended in 2TYAK media (2TY medium with 100 ug/ml Ampicillin and 25 ug/ml kanamycin) and grow overnight with 280 rpm shaking at 25° C. Cells were centrifuged down and supernatants with phage were used for following rounds of panning and ELISA screening.

### Example 3

### Whole Cell Panning

Full length Notch1 was transfected and expressed in 293 Flp-in Trex cells and Notch1 expression was induced by 1 ug/ml doxycycline for 24 hours. The detailed procedures for Notch1 cell line generation are described in other section. Panning was conducted using Notch1 expression cells for Notch1 binding phage selection and 293 Flp-in Trex cells used for non-specific phage de-selection control. Induction media for Notch1 expression contained advanced DMEM, 5% FBS (Tet defined), 2% L Glut, 1% Pen Strep, hygromycin (100 ug/ml), 1 ug/ml doxycycline.

Two days before panning, cells were seeded by splitting cells into fresh media. After 1 day of culture, cells were induced with doxycycline at 1 ug/ml concentration for 24 hours. Phage blocking was done using similar procedures as described in solid phase panning Both Notch1 expression cells and 293 TRex control cells were suspended from culture surface by add 5 ml of enzyme free cell dissociation buffer and leaving the flask at RT for 3-5 minutes to detach cells. Cell suspension was centrifuged down at 1800 rpm for 5 min, then removed media and re-suspended cells in 1 ml of fresh media with 5% fetal bovine serum (FBS) and 1×10̂7 cells.

To de-select phages binding to 293 TRex control cells, blocked phage was mixed with 293 TRex control cells (1×10̂7) with gentle mixing at 4° C. for 60 min. After the selection, phage supernatants were used to incubate with Notch1 expressing 293 cells for 2 hour at 4° C. with slow mixing. After the phage capture and incubation, cells were washed with 1 ml of PBS/0.1 Tween for 2 times and with 1 ml PBS for one time. Elution of cell Notch1 bound phages was done by adding 1 ml of 1× trypsin solution and incubating cells at 37° C. incubator for 30 min.

Procedures for phage rescue and amplification in TG1 E. coli cells are similar to that described in the example 2 for solid phase panning

### Example 4

ELISA with Phage Display ScFv and Recombinant Notch1 ECD Protein

Single E. coli colonies were grown on TYAG agar plates at 37° C., over night after infection of TG1 with panning eluted phages. Fresh TYAG media (270 μl/well) were inoculated with cells picked from single colony and cells were cultured at 37° C., 280 rpm for 4-5 hr, to OD600=0.5-1.0. Sixty micro-liter per well (60 ul/well) of M13K07 helper phage with titer of 10¹⁰ pfu/ml was added to TG1 cells and helper phage infection was conducted at 37° C. for lhr with 100 rpm shaking After helper phage infection, cells were centrifuged down at 3200 rpm for 10 min and supernatants were removed and cells were resuspended back into 150 ul of 2XTY media with 50 μg/ml ampicillin and 25 μg/ml kanamycin. The cells were cultured at 30° C., 280 rpm, over night.

After over night phage amplification, cells were centrifuged down at 3200 rpm for 15 min, and supernatants with phage were blocked with 5% milk blocker by mixing 40 ul blocker with 40 ul of phage supernatants for 1 hour at room temperature.

Recombinant human Notch1 extracellular domain protein (amino acid 19-526) from R&D Systems was used to coat maxisorp binding plates (Costa) at 4 μg/ml concentration in PBS over night at 4° C. Notch1 antigen needed per plate is 10 ml, 100 μl/well. Notch1 antigen coated plates were blocked with 200 ul of milk blocker solution (2% skim milk in PBS) each well at RT for 2 hour. The plates were washed with PBS/0.05% Tween 20 for 5 times and PBS for 3 times. Then the blocked phage solution was added to the Notch1 coating plate and incubated at room temperature for 2 hour. After incubation and washing steps, detection antibody for M13-phage with horse radish peroxidase (HRP) enzyme conjugation (NC9491398, Fisher/Amersham Bioscience) was added to plate at 100 μl/well in 1:2000 dilution with milk blocker. After 1 hour incubation at room temperature and washing step, 100 ul of HRP substrate, tetramethylbenzidine (TMB, Sigma) was added to each well and incubate for 5 min, then 50 μl of 0.5M H₂SO₄ was added to stop reaction and plates were read at 450nm for absorbance.

Vector phagemid without antibody display was used as assay control and positive binding clones were selected if signal/background is equal to or bigger than 5.

### Example 5

ELISA with Phage Display ScFv and Notch1 Expressing Cells

Notch1 expressing 293 Flp-TRex cells were cultured in advanced DMEM with 5% FBS (Tet defined), 2% L-Glut, 1% Pen/Strep, Hygromycin (100 ug/ml) and 293 Flp-TRex control cells were cultured in a similar media but with Zeocin (antibiotic 50 μg/ml) selection. Two days before ELISA, cells were detached from culture flask with Trypsin (0.25%) and cell number was counted using trypan blue staining method. In each well, 100 ul of cells (˜5×10E4 cells) were seeded in a 96-well cell culture plate (Costar, cat #3595) and incubated at 37° C., 5% CO₂ overnight. After the overnight culture, cells were induced with doxycyclin (2 μg/ml) for 24 hours and cells then were used for phage binding in ELISA.

Notch1 expression cells were fixed by adding 10% of buffered Formalin phosphate (Fisher, cat#SF100-4) and incubating at room temperature for 20 minutes. After fixing cells, 200 ul of wash buffer (from Superarray cat#FA001) per well was used to wash cells twice, 5 min incubation time for each wash. Quenching buffer (1% H2O2 and 0.1% NaN3 in 1× wash buffer) was added to cells and incubated at RT for 20 minutes and then cells were washed with PBS and blocked with 3% no fat dry milk in PBS at RT for 1 hour. Procedures for phage preparation are similar to that described in example 3 and phages were blocked with 3% milk blocker before adding to cells. Per well 100 μl of blocked phage was added and cells were incubated with phage at RT for 2 hours. After phage/cell incubation, plates were washed 3 times with washing buffer, 5 minutes incubation each time and final wash with PBS. Anti-M13 phage antibody with HRP was used for detection of cell bound phage. Details are also described in example 4.

### Example 6

### Sequencing Positive Clones and Analyzing Sequences Using IMGT Program

Notch1 binding clones were plated onto TYAG plates and incubated at 37° C. over night. Single colonies were picked out to grow in liquid medium over night and plasmid DNA were isolated using QIAGEN kit. Sequencing of ScFv using plasmid pCANTAB6 vector was conducted using following two primers and all sequences were confirmed with double strand sequencing.

Forward primer sequences are
5′-CAGGAAACAGCTATGAC-3′
Reverse primer sequences are
5′-GTCGTCTTTCCAGACGTTAGT-3′

A total of 147 unique sequences were identified from solid phase and whole cell panning (51 were Notch 1 binding specific and 96 clones can bind both Notch1 and Notch3 receptors)). The list of the amino acid, DNA, and CDR antibody sequences for both the heavy and light chain is included in Appendix III and IV. As indicated in the mentioned appendix, the selected clones were grouped in different buckets depending on their capability to bind by FACS either Notch1 (Appendix III) or Notch1/Notch3 (Appendix IV).

### Example 7

### FACS Screening of Cell Notch1 Binding Antibodies Using Phage Display ScFv

293 Flp-TRex Notch 1 cells were harvested and resuspended in PBS with 3% FBS at cell density approximately 1×10⁶/ml. Phage display ScFv was prepared by over night growth of TG1 cells with M13 helper phage infection and detailed procedures of phage amplification step are described in example 2. Over night grown phage display ScFv was pre-block in PBS, 3% FBS at RT for 1 hr and 100 μl of cells was incubated with the pre-blocked phages at RT for 45 min. Cells were transferred into FACS tubes and washed with 4 mL PBS, 3% FBS, then centrifuged at 1200 rpm for 10 minutes. Supernatant was removed and cells were stained with biotinylated anti-M13 phage antibody (Progen cat.no. 61597) for 45 min at RT. After washing cells with 4 mL of PBS, 3% FBS and collecting cells after centrifugation at 1200 rpm for 10 minutes, cells were stained with streptavidin-APC (Invitrogen 5868) for 30 min at RT. Cells were washed again with 4 mL PBS, 3%FBS, then centrifuged down at 1200 rpm for 10 minutes. Cell pellets were fixed in 1% formaldehyde and analyzed on a FACSCalibur (BD Biosciences) by collecting 30,000 events from gate 1.

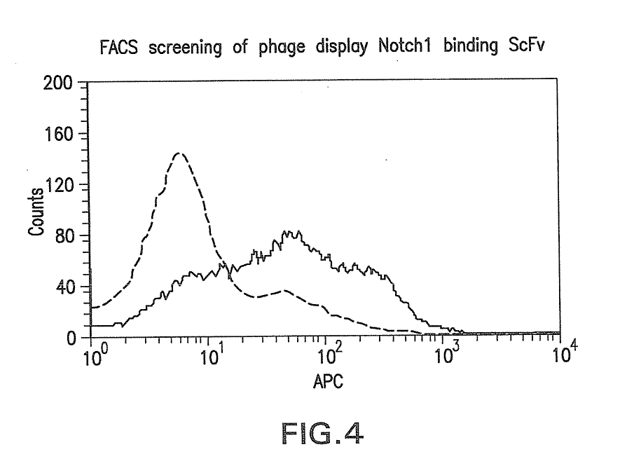

M13 phage without ScFv display on surface was used as negative control. FIG. 4 shows FACS graph for one of Notch1 binding antibody displayed as ScFv on phage.

### Example 8

Conversion of scFv Leads into IgGs for Further Characterization

scFv leads selected for IgG conversion. Following the library panning and screening described in earlier examples, a battery of scFv clones were selected for IgG conversion. The scFv variable region DNA and amino acid sequences for both the heavy and light chain of the antibodies are outlined in Appendix III and IV. All heavy chain variable regions were in-frame fused with human IgG1 constant region (Appendix V and VI), all light chain variable regions were in-frame fused with matching light chain constant regions, either lambda or kappa one (Appendix V and VI). The leader sequences were also in-frame fused with the variable regions accordingly. Their DNA and protein sequences are shown in Appendix VII and VIII, respectively.

IgG conversion vectors. The converted IgGs can be expressed using plasmid based vectors. The expression vectors were built such that they contain all the necessary components except the variable regions. In the basic vectors, the expression of both light and heavy chains was driven by human CMV promoter and bovine growth hormone polyadenylation signal. The leader sequence in the front mediated the secretion of antibodies into the culture medium. The heavy chain leader sequence was MEWSWVFLFFLSVTTGVHS whereas the light chain leader sequence was MSVPTQVLGLLLLWLTDARC (Appendix VII and VIII). The constant regions for the matching light or heavy chain (Appendix V and VI) were built in the vectors. In the case of light chain expression vectors, the constant region can be either lambda or kappa; In the case of heavy chain expression ones, the constant region can be different subclass isotype, e.g., IgG1 or IgG2. Between the leader sequence and the constant region, the intergenic sequences contains cloning sequences for seamless in-frame fusion of the incoming variable region with the leader sequence at its 5′-end and the constant region at its 3′-end using In-Fusion cloning strategy (Clontech). The expression vectors carry oriP from EBV viral genome for prolonged expression in 293EBNA cells and the bacterial sequences for kanamycin selection marker and replication origin in E. coli. When the variable regions were inserted, the IgGs can be directly expressed in mammalian cells. All the heavy chain variable regions were cloned into IgG1 expression vector (pV1JNSA-BF-HCG1) and the light chain variable regions were cloned into matching kappa or lambda expression vector (pV1JNSA-GS-FB-LCK or LCL).

IgG Conversion Cloning. The cloning procedure for the resulting antibody expression vectors is described below. The variable regions were PCR amplified. PCR reactions was carried out in a volume of 25 μl containing high fidelity PCR master mix, template volume 1 μl and forward and reverse primers: 1 μl each. PCR condition was 1 cycle of 94° C., 2 minutes; 25 cycles of 94° C., 1.5 minutes; 60° C., 1.5 minutes; 72° C., 1.5 minutes and 72° C., 7 minutes; 4° C. until removed. The PCR products were then digested with DpnI and purified with QIAquick plate kit (Qiagen). 100 ng of the corresponding previously linearized heavy chain or light chain vectors annealed to 10 ng of the PCR fragment with an In-Fusion reaction (Clontech IN-Fusion Dry-Down Cloning Kit). The reaction mixture is then transformed to XL2 Blue MRF' competent cells and plated overnight on Agar plates containing 50 ug/ml Kanamycin. Light chain constructs are then digested with HindIII+NotI and heavy chain constructs are digested with AspI+HindIII to check structure by restriction analysis. The DNA sequences for all the clones were confirmed by sequencing.

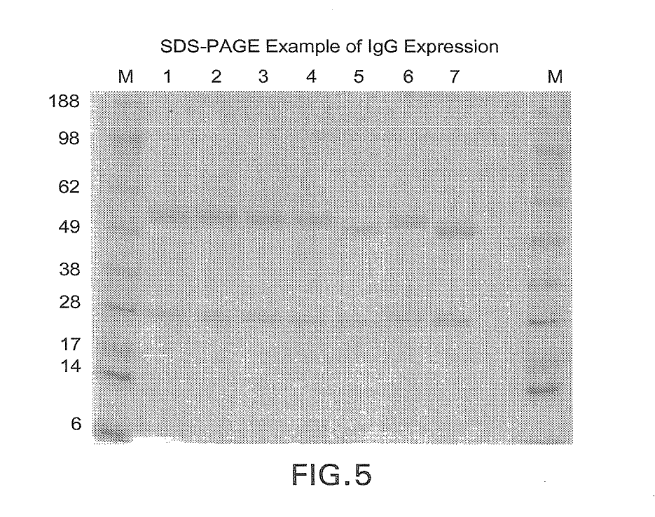

IgG Expression in Mammalian Cells, Purification and Characterization. Sequencing confirmed constructs of light chain and heavy chain DNA were transfected in 293 Freestyle cells or in EBNA monolayer cells. The 293 Freestyle cells were transfected using 293Transfectin from Invitrogen. EBNA monolayer cells were transfected using PEI based transfection reagents. Transfected cells were incubated at 37° C./5% CO2 for 7 days in Opti-MEM serum free medium (Invitrogen). The medium was collected, spin down and filtered through 0.22 um filtration system (Millipore), then concentrated by Centricon (Millipore). Concentrated medium were mixed 1:1 with binding buffer (Pierce), and then was loaded into pre-equilibrated protein A/G column (Pierce) or HI trap rProtein A FF from GE healthcare. Loaded column was washed with binding buffer and eluted with elution buffer (Pierce). Eluted antibody was neutralized immediately and dialyzed against buffer PBS for overnight. Dialyzed antibody was concentration with Amicon (Pierce) and protein concentration was determined by OD₂₈₀ₙₘ with the extinct coefficient of 1.34 mg/ml. Purification antibody was analyzed using SDS-PAGE (Invitrogen), or protein labchip (Caliper LifeSciences). SDS-PAGE was run under non-reduced conditions (FIG. 5).

### Example 9

### Notch1 Antibody Titration ELISA for Binding Affinity (EC50) and Epitope Determination

To prepare human Notch1 ECD domain proteins for titration ELISA, Notch1 ligand binding ECD (EGF like domain 1-13) was diluted in PBS and human Notch1 NRR antigen in TBS with 10 mM CaCl2 and working concentration for coating maxisorp plates was 5 ug/ml. Coating 96-well plates was done by adding 100 ul of Notch1 antigen solution to each well and incubating over night at 4° C. Plates were blocked with 300 ul of milk blocker per well and incubated for 2 hour at RT. Purified Notch1 binding antibodies IgG1 was diluted in series of 3-fold cross 12 wells in columns in milk blocker with starting concentrations from 50 ug/ml to 500 ug/ml (100 uL/well) depending on binding affinity. Anti-Notch1 antibody was incubated with coated Notch1 antigen for 2 hours at RT. After washing plates with PBS or TBS buffer, anti-human lambda light chain antibody with HRP was used for detection antibody and absorbance at 450 nM was read with a plate reader.

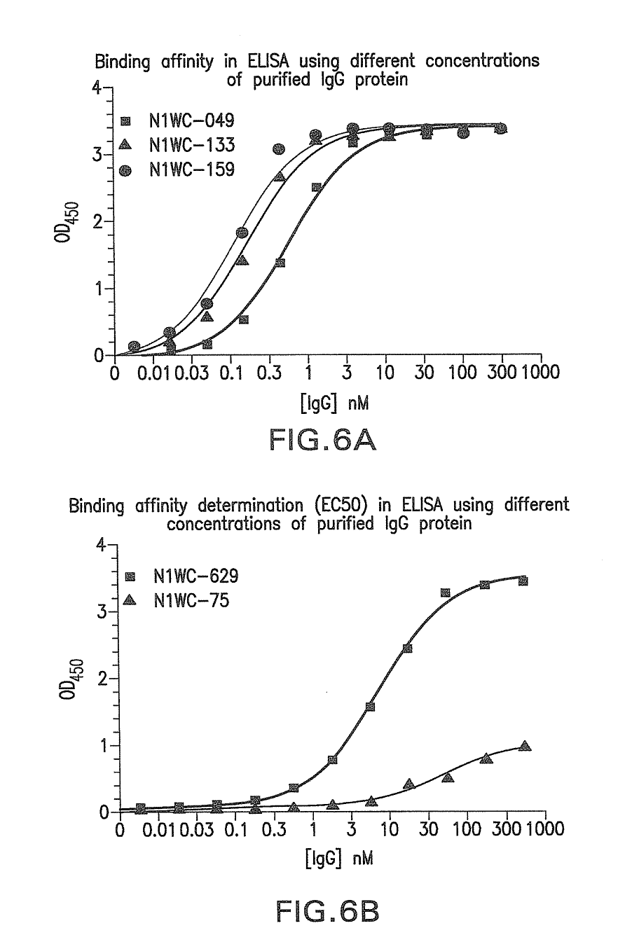

FIG. 6A and 6B show the binding curves of Notch 1 Mabs in different concentrations to ligand binding ECD domain protein (FIG. 6A) and LNR-HD domain protein (FIG. 6B), respectively.

To determine EC50 value, the data from titration ELISA was curve fitted using PRISM software and Table 1 shows EC values for selected Notch1 binding antibodies. Binding epitope was determined based on binding profiles assayed with two different Notch1 ECD domains in titration ELISA.

### Example 10

### Binding Kinetic Constants Determined Using BIAcore Analysis

All experiments were performed at 25° C. at a flow rate of 40 μl/min. To prepare a BIAcore assay, Notch1-ECD Fc fusion protein (Notch1/Fc) from R&D Systems (10 μg/ml each in acetate buffer, pH 4.0) was immobilized onto a carboxymethyl dextran sensorchip (CM5) using amine coupling procedures as described by the manufacturer. Close to 500 resonance units (RU) of Notch1/Fc were linked onto Flowcells (FC1, 2). FC1 was used as the reference cell. Specific signals were the results of differences of signals obtained on FC2 versus FC1. The analyte (Notch1 Mabs), was injected during 90 seconds at five different concentrations (50, 25, 12.5, 6.25 and 3.125 nM) in 0.5% P20, HBS-EP buffer. The dissociation phase of the analyte was monitored over a period of time ranging 10-30 minutes. Running buffer was also injected under the same conditions as a reference. After each cycle (antibody+analyte injection), both Flowcells were regenerated by injecting 20 to 45 μl of Glycine-HCl buffer, pH 1.5, to eliminate all Mabs and Notch1/Mab complexes captured on the sensorchip.

Binding kinetics constants of the anti-Notch1 Mab to Notch1/Fc were characterized by both association and dissociation rate constants kₐ and k_{d}, respectively. Equilibrium dissociation constant (KD) was calculated by the ratio between dissociation (koff) and association (kon) rate constants. Table 2 shows kinetic constants of Notch1 binding antibodies.

### Example 11

### Ligand Blocking Assay

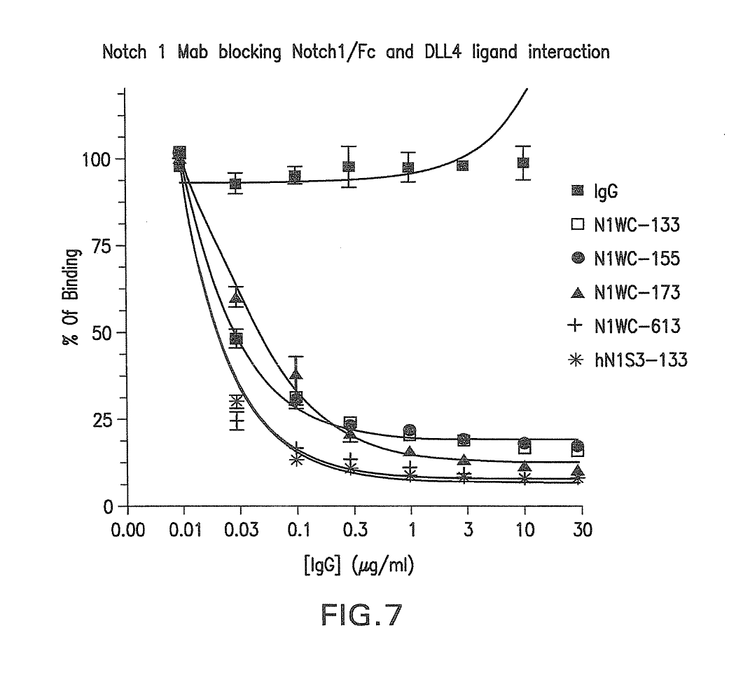

This assay was developed based on a dissociation-enhanced time resolved fluorometric assay (DELFIA) technology. Briefly, Notch ligand was coated in Maxisorp 96-well plates with 100 ul/well at 2 ug/ml concentration in D-PBS and incubated at 4° C. over night. The coated plates were blocked by adding 300 ul/well of 5% BSA in D-PBS blocker and incubating for 2 hours at room temperature. Notch1/Fc ECD protein was pre-labeled with europium (Eu) reagent according to manufacture suggested procedure (PerkinElmer). Notch1 monoclonal antibody in concentration titrations (0-30 mg/ml) was incubated with fixed amount (0.5 ug/ml) of Eu labeled Notch1/Fc for 2 hour with shaking before adding to DLL coated plate to block ligand binding site with antibody. After the incubation and blocking step, mixture of Notch1 antibody and Eu-Notch1/Fc was added to the Notch1 ligand coated plates and incubated at RT with slow shaking for lhour to capture Notch1/Fc protein through ligand receptor interaction. Then plates were washed with PBS-0.05%Tween for 5 times and with PBS for 3 times with a plate washer and 100 ul/well of enhancement solution was added for 5 minutes with shaking after washing step to release europium from complex of Eu-Notch1/Fc receptor and ligand. Fluorescence signals were read using Victor3-V plate reader (PerkinElmer) Inhibition of Notch1/Fc binding to ligand by Notch1 antibody was shown in FIG. 7 with increasing antibody concentrations corresponding to decreasing Eu-Notch1/Fc signal.

### Example 12

### Characterization of Notch Monoclonal Antibodies (Mabs) by FACS

To determine whether the Notch Mabs bind selectively to Notch1 on the surface of viable cells, FACS analysis was performed using dox-inducible 293 FlpIn (TREx) cells stably expressing the full-length Notch1, -2, -3, or -4 receptors, respectively. Notch 293 FlpIn (TREx) cells were treated with dox (2 ug/ml) for two days to induce Notch expression before collecting them for FACS surface expression analysis. The cells were harvested from tissue culture flasks using Trypsin for 15 min, centrifuged at 1200 rpm for 10 minutes and resuspended in 2 ml PBS with 2% fetal bovine serum (2% FBS/PBS). The cells were counted, adjusted to 10⁷ cells/ml and 0.1 ml of cells was incubated with 1 ug of each Mab for 40 minutes at 4° C. The cells were washed, resuspended in 0.1 ml 2% FBS/PBS and incubated with 1 ug R-PE-conjugated goat anti-human Ig (Jackson ImmunoResearch) for 30 minutes at 4° C. Cells were washed, resuspended in 0.3 ml PBS with 10% formaldehyde and analyzed on a FACSCalibur (Becton Dickinson, Mt View, Calif.).

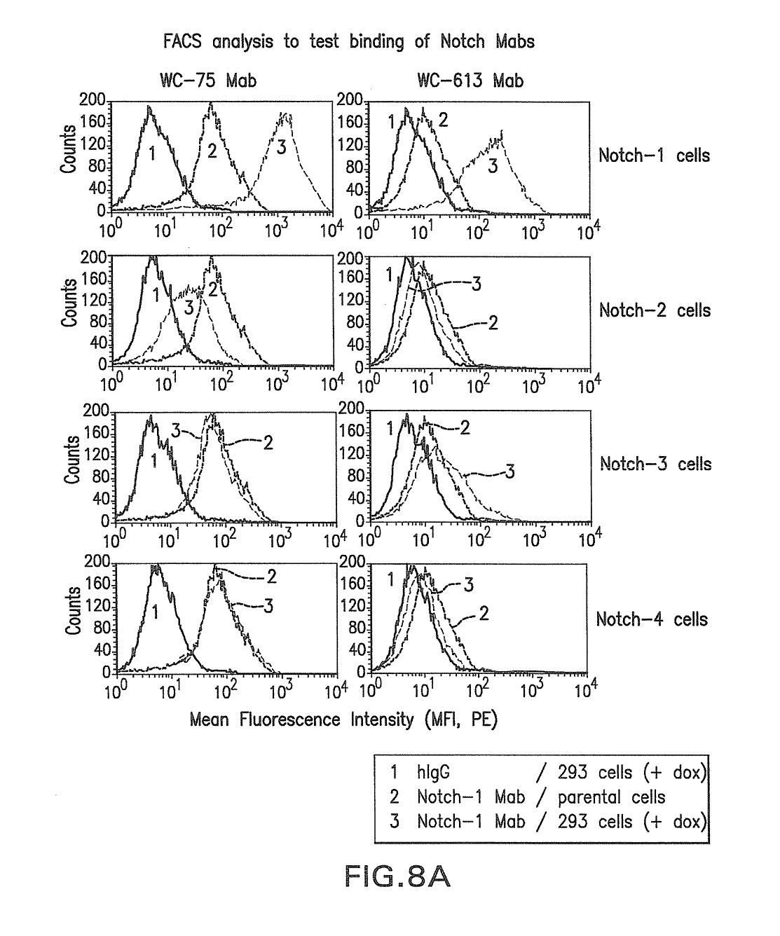

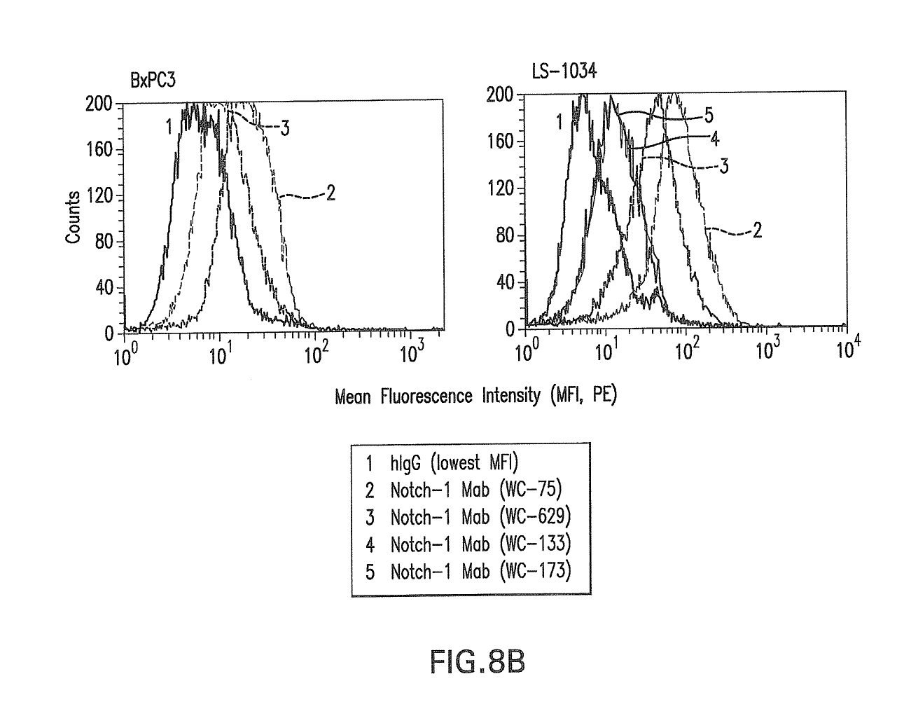

A panel of 26 Mabs was tested for the ability to bind 293 FlpIn (TREx) cells over-expressing Notch receptors. During the pre-screening of the scFvs generated by panning of the CAT libraries, these Mabs showed specificity and selectivity for Notch1 receptor. Antibodies binding to the Notch1 HD-LNR region (e.g., WC-75) showed a greater increase in fluorescence (MFI=1483), when compared to isotype control (MFI=9), than antibodies binding to the Notch1 ligand binding domain (LBD) (e.g., WC-613, MFI=211) (FIG. 8A). The most potent Notch1 Mabs (IC50s<25 nM) bind to the parental (e.g., WC-75, MFI=98) and to the Notch-2, -3, and -4 over-expressing 293 FlpIn (TREx) cells (FIG. 8A). These results suggest the presence of endogenous levels of Notch1 on the surface of the293 FlpIn (TREx) parental cells, which could not be detected previously using commercial anti-Notch1 antibodies. Also, the most potent Mabs showed binding to LS-1034 (e.g., WC-75, MFI=92) and BxPC3 (e.g., WC-75, MFI=23) cells (FIG. 8B) compared to the binding of IgG isotype control antibody (MFI=9). Analysis of protein expression by Western blot has previously shown the presence of Notch1 in LS-1034 and BxPC3 cancer cell lines (not shown).

### Example 13

### Functional Assays—Characterization of IgGs

Generation of mouse and human Notch antigens and stable cell lines. Chimeras of human Notch receptors were prepared by inserting the Gal-4 protein into the Notch intracellular domain (NICD) previously deleted of two (e.g., chimera Notch1-Gal4) or all (e.g., chimera Notch2-Gal4) ankryrin repeats and most of its RAM domain.

### Materials:

Human osteosarcoma cells: U2OS-FlpIn-TREx/human N1Gal4
   U2OS-FlpIn-TREx/human N2Gal4
   U2OS-FlpIn-TREx Trex/hN3Gal4 (BWH, Boston)*
Human osteosarcoma cells: U2OS-FlpIn-TREx/mouse N1
Mouse Fibroblast cells: 3T3-/Jag2 (BWH, Boston)*
DNA reporter gene: pFR-Luc (Stratagene, Cedar Crest, Tex.)
Transfection reagent Fugene 6 (Roche, Indianapolis, Ind.)
Bright-Glo Luciferase reagent (Promega, Madison, Wis.) * Brigham and Women's Hospital, Boston

### Methods:

a. Generation of Human Notch-Gal4 Chimeric U2OS Cell Lines

The full-length Notch1 and -2 (both with a c-terminal FLAG tag) receptors were synthesized by DNA 2.0 Technologies. In the Notch1 chimera, the Gal4 binding domain (Gal4-GVP plasmid, Invitrogen, Carlsbad, Calif.) was PCR amplified and inserted into the Notch1-FLAG gene at the Bsu36I site (in the RAM sequence of NICD) and Stul site (in the second Ankyrin repeat of NICD)). This insertion replaces most of the RAM sequence and the two Ankyrin repeats with the Gal4 binding domain sequence. In the Notch-2 chimera, the Gal4 binding domain was PCR amplified and inserted into the Notch2-FLAG gene at the MscI site (in the RAM sequence of NICD) and MscI site (in the Ankyrin repeat region of NICD). This insertion replaces most of the RAM sequence and all of the Ankyrin repeats with the Gal4 binding domain sequence. Both chimeric genes, Notch1 and -2 Gal4, were subcloned into pcDNA5 FRT TO vector (Invitrogen) and used to create stable U2OS cell lines over-expressing the Notch1 and -2-Gal4 chimeric receptors. U2OS FlpIn (TREx) parental cells (obtained from the Brigham and Women's Hospital, Boston) were stably transfected using the Fugene transfectant reagent and maintained in accordance with Invitrogen's 293 FlpIn (Trex) cell line protocol. Cells were selected for and maintained in DMEM high glucose (Invitrogen) with 10% FBS (Hyclone, Logan, Utah), 1X Pen-Strep (Mediatech, Herndon, Va.), and 100 ug/ml Hygromycin (Mediatech).

b. Generation of Full-Length Mouse Notch1 U2OS Cell Lines

The full-length mouse Notch1 receptor was synthesized by DNA 2.0 Technologies and subcloned into the Hind III and Not I restriction sides of the pcDNA5 FRT TO vector (Invitrogen) and used to create stable U2OS FlpIn TREx cell lines over-expressing mouse Notch1 receptors. Cells were stably transfected using the Fugene transfectant reagent and maintained in accordance with Invitrogen's 293 FlpIn (TREx) cell line protocol. Cells were selected for and maintained in DMEM high glucose with 10% FBS, 1× Pen-Strep, and 100 ug/ml Hygromycin.

c. Selectivity and Potency of Notch Antibodies Using Co-Culture Reporter Assays

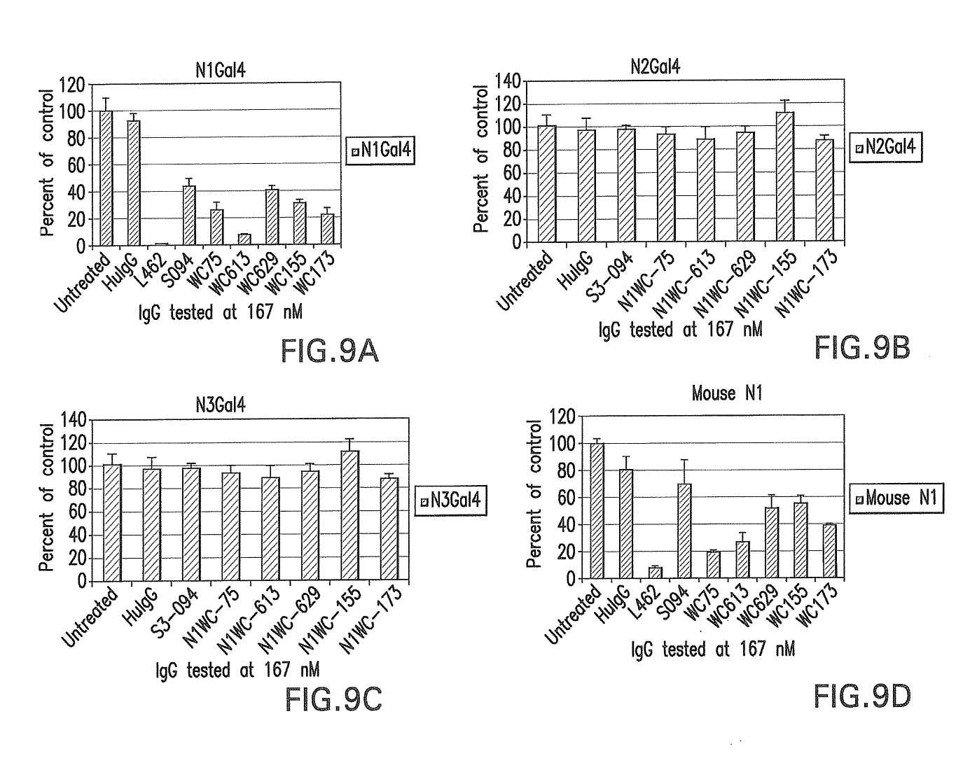

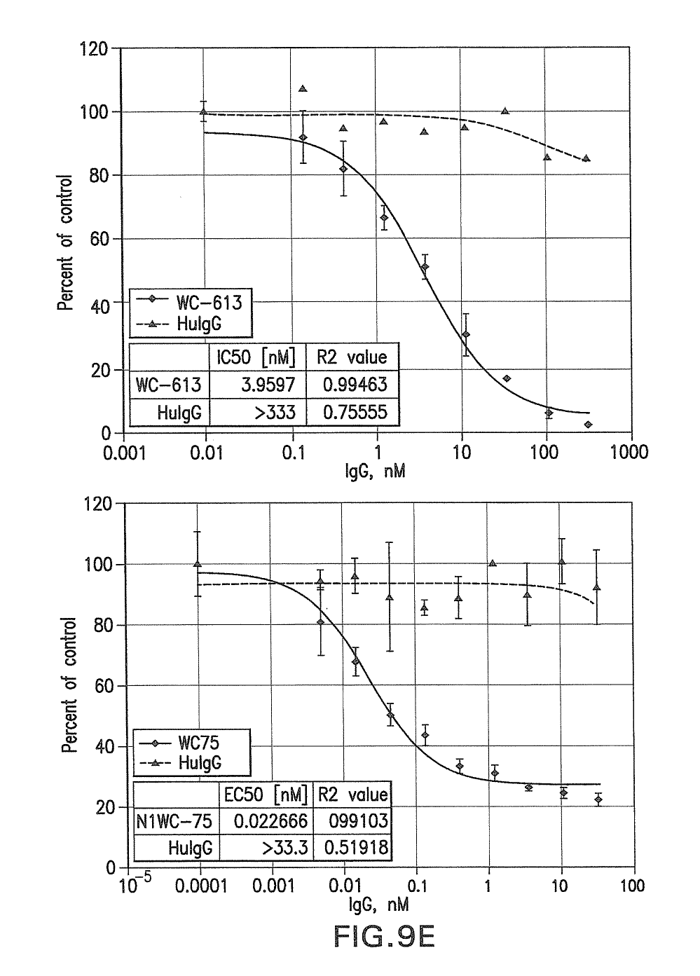

The ability of anti-Notch Mabs to antagonize the release of the ligand-dependent Notch Intracellular Domain (NICD) of Notch receptors was tested in a 96-well plate format using U2OS FlpIn (TREx)-Notch-Gal4 cells transfected with a pFR-Luc reporter plasmid (Stratagene) and co-cultured for 24 hours with ligand-overexpressing 3T3 cells. Each of the U2OS FlpIn TREx-Notch cell lines were generated by stably transfecting pcDNA5 plasmids encoding the respective human Notch-Gal4 chimera receptors (Notch1, -2, and -3). The full-length mouse Notch1 stably transfected in U2OS FlpIn (TREx) cells was generated using the same transfection protocol as the human Notch chimera receptors. For the assay, U20S-Notch-Gal4 cells were transiently transfected with a pFR-Luc reporter plasmid (Stratagene) driven by a 5X-UAS Gal-4-interacting promoter on day 1; then, on day 2 cells were treated with dox in the presence or absence of Notch Mabs and IgG control, and co-cultured for 24 hours with Jag2-overexpressing 3T3 cells (1.5×10⁴/well) seeded in 96-well plates. Activity of luciferase was measured in cell lysates with Bright-Glo assay kit (Promega). In the mouse Notch1 reporter assay, transactivation of the luciferase reporter gene was driven by the 4× promoter which binds to the endogenous CSL transcription factor. The anti-Notch Mabs of the invention selectively blocked Notch1 (FIG. 9A), but not Notch-2 (FIG. 9B) or Notch-3 (FIG. 9C) luciferase activity. The potency of the Mabs (IC₅₀ₛ) was determined using serial dilutions of the Mabs to generate sigmoid IC₅₀ curves. IC₅₀ values were determined using a 3-parameter logistic curve fit with the top fixed at 100. The Mabs inhibit specific Notch1 signaling with a wide range of potencies varying from IC₅₀ values of less than 1 nM to greater than 100 nM (FIG. 9E, Table 3). Some of the Mabs did not block Notch1 signaling in the reporter assay even though they showed binding to Notch1 by FACS (not shown). The Mabs were also tested for their capacity to block signaling in U2OS FlpIn TREx cells stably transfected with the mouse Notch1 receptor. In these experiments, the Notch antibodies at a concentration of 166 nM blocked luciferase signal in the range from 26% to 81% of the maximal fold stimulation in the co-culture conditions without antibody treatment (FIG. 9D).

### Example 14

### The 10k Membranome Collection of Phage-Ab

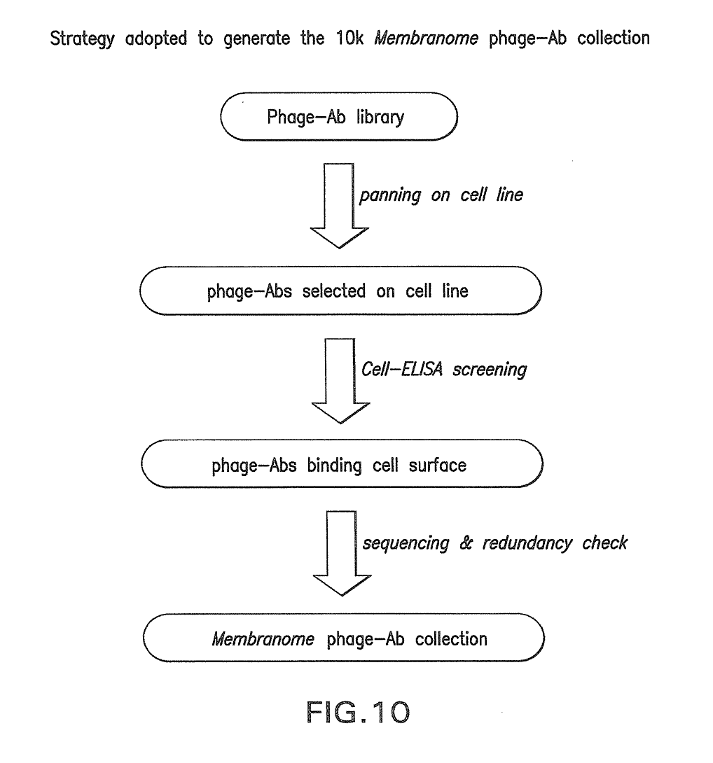

We define as Membranome the ensemble of human genes coding for proteins associated to the cell membrane. We built up a collection of phage-Ab binding the Membranome proteins by high-throughput in vitro screening of naïve phage-Ab libraries using various cell lines as “selectors”. Membrane Proteins (MPs) expressed on the cell surface maintain their native features: folding, post-translation modification, expression of splicing variants and formation of multimeric complexes. In addition, cultured cell lines are reagents easily available with reproducible properties. The workflow adopted to build up the Membranome phage-Ab collection is schematically described in FIG. 10. Three naïve phage-Ab libraries were used in this work which have been derived from B cells of human healthy donors and include over 10¹⁰ different clones each (Cambridge Antibody Technology DP47, BMV, CS). These libraries were independently panned on 64 different human cell lines derived from 25 different tissues (see Table 4). Phage-Abs from each selected pool were individually screened by cell-ELISA for their ability to bind the selector cell line. Positive clones with a novel VH-CDR3 sequence were progressively added to the 10k Membranome phage-Ab collection. Automation of this process enabled a high throughput screening.

Briefly, libraries of scFv displayed on filamentous phage were obtained from Cambridge Antibody Technology. These libraries (DP47, BMV and CS) were derived from B cells of healthy donors and each of them includes more than 10¹⁰ different clones each. Each phage-Ab library (about 10¹¹ TU) was independently panned on 64 different cell lines (listed in Table 4). In each selection phage libraries were incubated with MPBS (3% powdered milk in PBS solution) for 30 min at room temperature (RT). Human cells used in the selection were detached from the plate using 2.5 mM EDTA in PBS and re-suspended to a final concentration of 1×10⁷ cells/mL. Following centrifugation, cells were re-suspended with pre-adsorbed phage-Abs solution and incubated for 1 hour at RT. After extensive washing with PBS, cell-bound phage were centrifuged 5 min at 2 krpm and resuspended in 800 μL of trypsin solution (1 μg/ml in sodium phosphate). E. Coli TG1 cells (New England Biolabs, Beverly, Mass.) were infected with eluted phage and plated on 2XTY agar containing 2% glucose and 100 μg/mL ampicillin (2XTYAG). Phage rescue and amplification was carried out as described (Hegmans et al 2002; J Immunol Methods 262: 191-204. The selected phage were panned again on the same cell line. A variable number of clones (in the range 100-1,000) were randomly chosen form the pool of selected phage and tested by phage ELISA for their ability to bind the same cell line.

a. Screening the Membranome Collection for Notch1 Ligands

The 10k Membranome phage-Ab collection is a “specialized”, low-complexity library composed by clones binding receptors expressed in their native form on the cell surface. This collection can be surveyed to rapidly identify antibodies binding epitopes of known cell-surface receptors. We screened the Membranome to identify phage-Abs binding the extra-cellular domain of Notch1. As a selector molecule we decided to avoid using a recombinant extra-cellular domain of the receptor. We chose to use the Notch1 receptor expressed in its natural environment, i.e. on the cell surface. This format is anticipated to preserve the active conformation of the receptor and include all the post-translational modifications. Using a Notch1 expressing cell implies, however, that many other different receptors will be displayed on the cell surface. We therefore used a differential selection system to focus our screening on Notch1 receptor. A stable cell line pcDNA5/FRT/TO-HN1+FLAG expressing Notch1 under the control of doxycyclin-responsive promoter (293_N1) was used after doxycyclin induction for 48 hrs at 100 ug/ml (test sample). The corresponding parental cell line 293TREX FLIP was used as control (293; reference sample). Panning the Membranome phage-Ab collection on test and on reference samples generated two phage populations. The goal is to identify clones preferentially or exclusively present in the test as compared to the reference population.

We estimate that the complexity of each pool is about 5,000 clones. This number makes very difficult testing the binding specificity of individual clones from each of the two populations and would anyway require a large number of transfected cells

As an alternative approach, sequencing a number of clones from the test and the reference population of phage-Ab would reveal clones which are differentially represented in the two pools. Clearly, a statistically significant comparison of the two populations would require a very large number of sequences. To efficiently acquire this information we developed a novel DNA-based screening technology we called tagArray.

First we designed a set of short oligonucleotides that could be used as tags and cloned each single tag into each phagemid coding for a phage-Ab. These tags were cloned in each phagemid coding for a phage-Ab. In this way we generate an association between a specific tag and a specific scFv sequence. This made possible to measure the frequency of each scFv clone in a population by measuring the frequency of the associated tag. The latter can be measured en masse by DNA microarray analysis.

The tagArray technology enables the efficient and sensitive comparative analysis of the entire populations using minimal amounts of selector samples. The screening protocol articulates in four steps: i) the tagged 10k Membranome phage population was panned in parallel on test and reference sample generating two distinct phage populations (Notch and mock phage population, respectively); ii) tag sequences were amplified from the Notch and the mock phage populations, labeled with Cy5 or Cy3 fluorochromes and hybridized on a double channel DNA microarray containing the complete repertoire of tag sequences; iii) analysis of the hybridization data identified the tags differentially represented in the two populations; iv) the scFv associated to the relevant tags are retrieved from the phage population.

b. TagArray Repertoire

There were identified 12 sequences sharing the following properties: i) 7 nucleotides long; ii) G2W5 base composition; iii) do not contain G-C palindromes; iv) do not contain self complementary sequences; v) have the same melting temperature, which is 10° C. lower as compared to each of the other 11 sequences. By combining 4 of these building blocks we generated a repertoire of 20,736 (12⁴) sequences of 28 nucleotides. Each member of this repertoire has a 10° C. difference or higher in Tm with any other member of the repertoire.

c. Tagging 10k Membranome

We synthesized 2 sets of 144 oligos with the following structure

Forward
5′-[BLOCK-C][BLOCK-D] GAACGACCGAGCGCAGCGAGTCA-3′

BLOCK indicates the upper strand of any of the 12 sequence we identified
The sequence at its 3′ is complementary to the region of the pCANTAB vector

Reverse
3′-[block-a][block-b]-GGCTGCGGCGAAGCGGTATCAGCT-5′

block indicates the lower strand of any of the 12 sequences we identified
The sequence at its 3′ is complementary to the region of the pCANTAB

PCANTAB
AGCTGATACCGCTCGCCGCAGCCGAACGACCGAGCGCAGCGAGTCA

[symbol]

We assembled an inverse-PCR reaction in 96 well plates containing a unique combination of the forward and reverse primers (5pmols each), 3% DMSO, 200 nM dNTPs, 0.65 units Taq polymerase in 50 uL final volume of HF buffer). Individual bacterial clones from the Membranome collection were included in each well. And processed according to the following thermal profile (98° C. for 3; 98° C. for 30″, 68° C. for 30″, 72° C. for 3′ (24 cycleS); 72° C. for 10″. About 5 uL of each PCR product was digested with DpnI for 2 hrs at 37° C. and inactivated for 20 min at 80° C. An aliquot of the reaction was then ligated overnight at 14° C. with T4 DNA ligase. Following ligase inactivation, the mix was electroporated in TG1 cells which were incubated overnight at 30° C.

d. Panning 10k Membranome

Phage from the Membranome collection, 293_N1 cells induced with doxycyclin for 48 hrs at 100 ug/ml and 293 cells were incubated in PBSM (3% non-fat dry milk in PBS) for 30 min. Then about 1×10exp10 TU from the 10k Membranome phage-Ab collection were panned for 30 min at 37° C. with 293-N1 or 293 cells. Following extensive washing, cell-bound phage were eluted by proteolytic cleavage and amplified by infection of TG1 cells.

e. Amplification Tag Sequences

The Ampicillin-resistant bacterial colonies were collected and phagemid DNA purified by DNA purification kit (Qiagen GmbH, Hilden, Germany). Tag sequences were amplified by PCR. Forward primer was 5′-labelled with Cy5 or Cy3 dyes. Reaction mix included 30 ng phagemid DNA, 5 μM labeled forward and reverse primers, 200 uM dNTPs and 2.5 units Taq polymerase (Promega, Madison, Wis.) in a final 50 μL volume. Nine explicitly tagged-phagemids with different relative ratios and absolute amounts were added to the reaction. These “spike in” probes allowed monitoring that the amplification process maintained the original relative frequencies of the clones in the amplified population. The amplification product was first purified from salts, free primers and dNTPs by a commercially available removal kit (Qiagen GmbH, Hilden, Germany). An aliquot was then run onto an agarose gel and the Cy5- or Cy3-labeled fragment was quantified by a fluorescence scanner (Typhoon; Perkin Elmer, Waltham, Mass.).

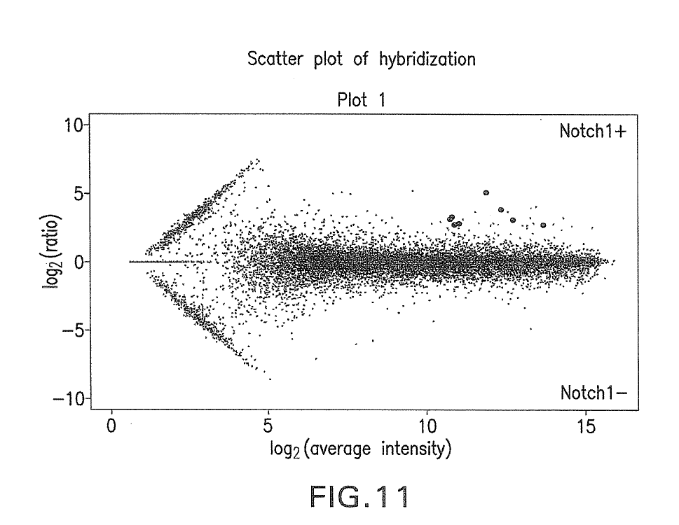

The Hybridization mix was assembled in 490 μL final volume containing 3.5 mM Cy5- and Cy-3 labelled probes, 0.5 μM blocker-1 and reverse primer. The latter perfectly hybridizes to the phagemid sequences flanking the tag, thus allowing the tag to interact with complementary tag sequences immobilized on the microArray chip (Agilent Technologies, Palo Alto, Calif.). The reaction mix was incubated at 99.9° C. for 2 minutes and then snap-cooled by transferring back in ice for 1 min. The mix was then applied onto tagArray_{—}44k chip and incubated at 64° C. for 15 hours in a rotating chamber. Then the slide was then washed at RT in 6× SSPEL (6× SSPE, 0.005% N-Lauroylsarcosine), 0.06× SSPEL, rinsed in stabilization and drying solution (Agilent Technologies, Palo Alto, Calif.) and analyzed with Agilent scanner. Images were acquired at 10 μm resolution using an Agilent scanner, with the XDR option enabled (eXtended Dynamic Range: for each slide two images were generated with photomultiplier tube voltages of 100 and 10, respectively). Images were then processed using the Feature Extraction software (v 9.1, Agilent Technologies, Palo Alto, Calif.) generating the net signal for each channel (Cy3 and Cy5) and p-values after background and dye-bias correction. Results reported were obtained by combining data from two independent fluor reversal experiments. The scatter plot derived from this analysis is reported in FIG. 11. Each dot is defined by the log 2 of its ratio between the 293-Notch and the 293 derived tag probes, and by the log 2 of the average of the same values. We filtered the data arbitrarily selecting the following criteria: p<10exp-4, log 2(ratio)>2.6 and log 2(average intensity)>10.7. The orange dots in FIG. 11 refer to clones which fulfilled these criteria. We selected 14 clones with highest ratio and analyzed the corresponding phage-Ab for their binding to 293-N1 and 293 cells by flow cytometry. These clones were converted to IgGs as described in example 16 to confirm binding by FACS and reporter activity in functional assays (Table 5).

### Example 15

### Analysis of the Selected Clones

a. Whole-Cell Phage-ELISA

Clones were analyzed as described in Section I-Example 5. Phage-Abs whose binding to the target cell line measured by A_{450 nm}-A_{620 nm} was at least three fold higher than that observed with an unrelated phage and higher than 0.3 units were defined as positive.

b. FACS Analysis (IgG Format)

Clones were analyzed as described in Section I-Example 12. We analyzed the corresponding Mabs for their binding to 293-N1, -N3 and 293 parental cells by flow cytometry. Three clones were N1-specific (see Table 5), and three (e.g., C2-514, D-268, and A673-C2-110) did cross-react with Notch-3 expressing cells.

c. Reporter Assays

The Abs selected for conversion were tested for their ability to inhibit Notch1, -2, and -3 signaling as described in the Example 13. Results are reported in the Table 5

### Example 16

Conversion of scFv Leads into IgGs

The conversion to IgG of the scFv selected clones (based on FACS analysis) was performed as described in Example 8. The scFv variable region DNA and aminoacid sequences are outlined in Appendix IX. As indicated, the selected clones were grouped in different buckets depending on their capability to bind or not bind the negative regulatory region (NRR) of the Notch1 receptor.

### Example 17

### Activity of Antibodies Against Notch-1 Receptors Bearing NRR Mutations

Notch-1 receptors bearing different T-ALL-associated mutations within the negative regulatory region (NNR) typically exhibit ligand-independent activation (i.e., spontaneous release of NICD). Ligand-independent signaling by these mutated forms of Notch-1 was evaluated in both the presence and absence of antibodies of the invention that bind to the negative regulatory region (NNR) of the receptor.

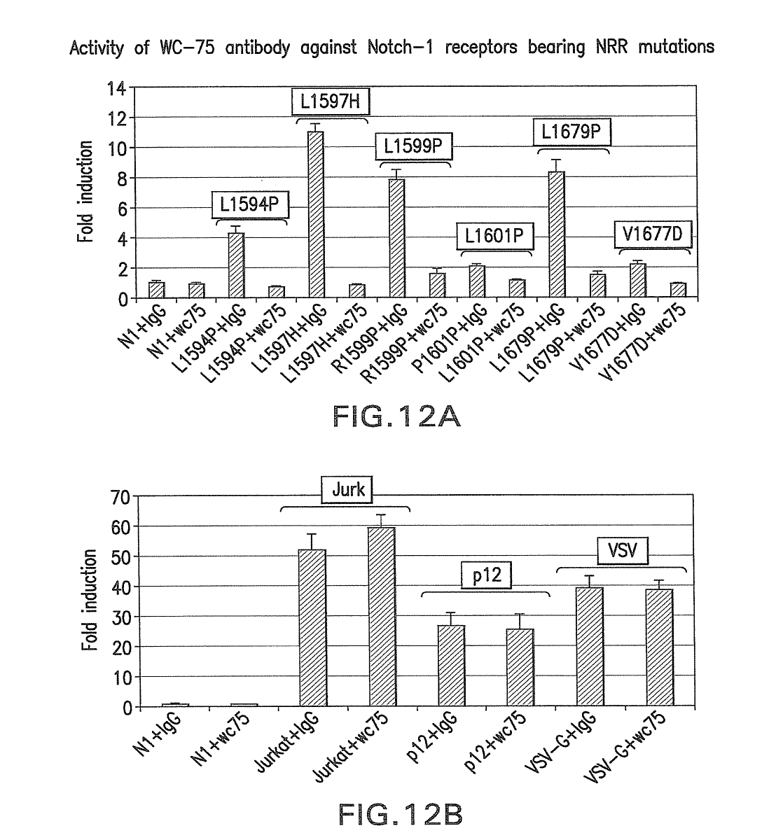

In a 96-well format, U2OS cells were transiently transfected with full-length Notch-1 cDNA constructs encoding specific mutations that cause spontaneous activation of the receptor and the consequent activation of the Notch signaling pathway. Immediately post-transfection with the mutated Notch receptor and the 4XCSL-luciferase reporter gene, inhibitory or control antibodies (10 ug/ml) were added and the luciferase signal was measured in cell lysates at 24 h with Bright-Glo assay kit (Promega). U2OS cells transient transfected with the wild-type receptor were used as the baseline control to express fold-induction secondary to spontaneous release of NICD. The intrinsic activity of the different receptors bearing Class I mutations (i.e., those that promote heterodimer dissociation) was very sensitive to antibodies (e.g., WC-75) binding to the NRR region (FIG. 12A). In contrast, signaling by Notch1 receptors with Class II mutations (i.e., tandem insertions that duplicate the S2 cleavage) was not significantly affected by treatment with these antibodies (FIG. 12B).

### Summary of Tables:

Table 1: Notch1 antibody EC₅₀ values calculated using titration ELISA
Table 2: Notch1 antibody kinetic constants calculated using BIAcore analysis
Table 3: Summary of monoclonal antibody activity
Table 4: Human cell lines used for selecting Membranone phage-Ab collection
Table 5: Summary of monoclonal antibody activity—from Membranome panning

### Summary of Appendices:

Appendix I: Notch1 DNA Sequence (Notch1+FLAG TAG)
Appendix II: Notch1 Protein Sequence (Notch1+FLAG TAG)
Appendix III: Group I antibodies-Notch1 specific—Generated directly from a phage display library
   A. Amino Acid Sequence
      A.1. Binding to LBD (N1-EGF 1-13) (SEQ ID NOS: 3-22 Hc & 23-42 Lc)
      A.2. Binding to NRR (SEQ ID NOS: 43-45 Hc & 46-28 Lc)
      A.3. Additional Notch Antibodies
   B. DNA Sequence
      B.1. Binding to LBD (N1-EGF 1-13) (SEQ ID NOS: 105-124 Hc & 125-144 Lc)
      B.2. Binding to NRR (SEQ ID NOS: 145-147 Hc and 148-150 Lc)
      B.3. Additional Notch Antibodies
   C. CDR (Amino Acid Sequence)
      C.1. Binding to LBD (N1-EGF 1-13)
      C.2. Binding to NRR
      C.3. Additional Notch Antibodies
Appendix IV: Group II antibodies—Bind to both Notch1/Notch3
   A. Amino Acid Sequence
   B. DNA Sequence
   C. CDR (Amino Acid Sequence)
Appendix V: Constant Region DNA Sequences
Appendix VI: Constant Region Protein Sequences
Appendix VII: Leader DNA Sequences
Appendix VIII: Leader Protein Sequences
Appendix IX: Group I Antibodies-Notch1 specific—Generated from the Membranome library
   A. Amino Acid Sequence
      A.1. Binding to NRR
      A.2. Additional Notch Antibodies
   B. DNA Sequence
      B.1. Binding to NRR
      B.2. Additional Notch Antibodies
   C. CDR (Amino Acid Sequence)
      C.1. Binding to NRR
      C.2. Additional Notch Antibodies

TABLE 1
Notch1 antibody EC₅₀ values calculated using a titration ELISA

| Mab Name | Binding Epitope | Binding EC50, nM |
|---|---|---|
| WC-75 | LNR-HD | 62.62 |
| WC-629 | LNR-HD | 5.50 |
| WC-133 | Notch1 (EGF 1-13) | 0.17 |
| WC-159 | Notch1 (EGF 1-13) | 0.12 |
| WC-49 | Notch1 (EGF 1-13) | 0.60 |

TABLE 2
Notch1 antibody kinetic constants calculated using BIAcore analysis

| | 1:1 binding | | |
|---|---|---|---|
| Mab (ID) | Kon (1/M · s) | Koff (1/s) | K_{D} (M) |
| HN1S3-133 | 6.16E+05 | 4.00E−04 | 6.49E−10 |
| HN1S3-147 | 1.06E+06 | 2.85E−04 | 2.69E−10 |
| HN1S3-13 | 5.97E+05 | 0.002937 | 4.92E−09 |
| HN1S3-56 | 5.17E+05 | 0.002918 | 5.65E−09 |
| HN1S3-94 | 4.20E+05 | 0.001632 | 3.89E−09 |
| HN1S3-9 | 5.13E+05 | 0.002681 | 5.23E−09 |
| hN1wc-97 | 5.56E+04 | 4.38E−04 | 7.87E−09 |
| hN1wc-155 | 1.66E+05 | 2.90E−04 | 1.74E−09 |
| hN1wc-168 | 4.86E+05 | 0.002281 | 4.70E−09 |
| hN1wc-173 | 1.37E+05 | 4.47E−04 | 3.27E−09 |
| hN1wc-179 | 4.93E+05 | 0.003628 | 7.36E−09 |
| hN1wc-613 | 2.37E+05 | 2.58E−04 | 1.09E−09 |
| hN1wc-49 | 9.89E+05 | 0.1605 | 1.62E−07 |
| Hn1WC-133 | 1.57E+05 | 2.60E−04 | 1.66E−09 |

TABLE 3

| | | hN1 | | | |
|---|---|---|---|---|---|
| Mab ID | hN1-FACS^{a, b} | (IC₅₀, nM) | hN2^{c} | hN3^{c} | mN1^{d} |
| WC-75 | ++++ | 0.023 | <10% | <10% | +++ |
| WC-629 | +++ | 2.1 | <10% | <10% | ++ |
| WC-613 | ++ | 3.9 | <10% | <10% | +++ |
| S3-009 | ++ | 12 | <10% | <20% | ++ |
| S3-094 | ++ | 13 | <10% | <10% | ++ |
| WC-133 | ++ | 15 | <10% | <10% | ++ |
| WC-173 | ++ | 21 | <20% | <20% | +++ |
| S3-013 | + | 25 | <10% | <10% | + |
| S3-056 | + | 35 | <10% | <10% | ++ |
| S3-011 | ++ | 50 | <10% | <10% | + |
| WC-155 | ++ | 61 | <10% | <10% | ++ |
| WC-168 | + | 114 | <10% | <10% | ++ |
| WC-159 | +++ | 147 | <10% | <10% | ++ |
| WC-97 | + | 152 | <10% | <10% | ++ |

^{a}hN1, mN1 represent human and mouse Notch1, respectively.
^{b}Binding of Notch Mabs to 293 FlipIn (TREx) cells stably expressing hN1: (−) MFI < 11; (+) MFI = 11-100; (++) MFI = 101-500; (+++) MFI = 501-999; (++++) MFI > 1000
^{c}Inhibition of Luc activity at [166 nM] Mab in the Notch human reporter assay: (−) <20%;
^{d}Inhibition of Luc activity at [166 nM] Mab in the mouse reporter assay: (−) <20%; (+) = 21-30%; (++) = 31-60%; (+++) = 61-90%; (++++) >90%

TABLE 4
Human cell lines used for selecting Membranome phage-Ab collection

| Cell line^{a} | Tissue | Clones |
|---|---|---|
| A549 | lung | 137 |
| HCT-116 | colon | 50 |
| HL-60 | blood | 57 |
| SW480 | colon | 302 |
| G-361 | skin | 17 |
| T-47D | breast | 46 |
| HT-3 | cervix | 4 |
| WiDr | colon | 258 |
| MDA-MB-231 | breast | 118 |
| MDA-MB-468 | breast | 35 |
| BT-474 | breast | 9 |
| 143B | bone | 59 |
| AGS | stomach | 268 |
| SW620 | colon | 247 |
| LN405 | brain | 34 |
| PerC-6 | retina | 39 |
| HeLa | cervix | 169 |
| RKO | | 192 |
| A-498 | kidney | 2 |
| MIA-PaCa-2 | pancreas | 232 |
| MSTO-211H | lung | 57 |
| SK-CO1 | colon | 151 |
| MG63 | bone | 440 |
| CAL-120 | breast | 426 |
| SK-N-AS | brain | 350 |
| A-375 | skin | 95 |
| PANC02.03 | pancreas | 203 |
| LNZTA3WT4 | brain | 146 |
| A498 | kidney | 2 |
| U-2OS | bone | 181 |
| 5637 | urinary bladder | 114 |
| A-673 | muscle | 381 |
| G-402 | kidney | 340 |
| JEG-3 | placenta | 210 |
| T98G | brain | 251 |
| MCF7 | breast | 303 |
| NCI-H292 | lung | 51 |
| KATOIII | stomach | 197 |
| KG-1 | bone marrow | 88 |
| PC-3M-luc-C6 | prostate | 231 |
| CAL-27 | tongue | 151 |
| Caov-3 | ovary | 180 |
| HEK293T/17 | kidney | 99 |
| HepG2 | liver | 367 |
| K-562 | bone marrow | 68 |
| A-204 | muscle | 323 |
| A2780 | ovary | 197 |
| A2058 | skin | 224 |
| OAW42 | ovary | 67 |
| C32 | skin | 155 |
| AN3CA | uterus | 129 |
| J82 | urinary bladder | 55 |
| NCI-H460 | lung | 127 |
| SKOV-3 | ovary | 244 |
| DAOY | brain | 129 |
| A-431 | skin | 28 |
| HPAF-II | pancreas | 107 |
| LS1034 | cecum | 48 |
| HUVEC | umbelicalvein | 198 |
| Hs766T | pancreas | 31 |
| HUH7.5 | liver | 395 |
| | Total | 9,925 |

^{a}Cell lines are listed according to the order they have been used to select the Membranome phage-Ab collection.

TABLE 5
Summary of monoclonal antibody activity -Membranome panning

| | hN1- | hN1^{c} | | |
|---|---|---|---|---|
| Mab ID | FACS^{a, b} | (IC₅₀, nM) | hN2^{c, d} | hN3^{c, d} |
| A2780-C2-16 | ++++ | 0.583 | <15% | <15% |
| JEG-C2-11 | ++++ | 0.299 | <5% | <5% |
| C2-384 | +++ | >333 | <5% | <5% |
| C2-514 | ++++ | >333 | <5% | <5% |
| D2-268 | ++++ | >333 | <5% | <5% |
| A673-C2-110 | ++ | >333 | <15% | <10% |

^{a}hN1 represents human Notch1
^{b}Binding of Notch Mabs to 293 FlipIn (TREx) cells stably expressing hN1: (−) MFI < 11; (+) MFI = 11-100; (++) MFI = 101-500; (+++) MFI = 501-999; (++++) MFI > 1000
^{c}Reporter assays
^{d}Inhibition of Luc activity at [166 nM] Mab: (−) ≦20%

### APPENDIX

APPENDIX I
Notch1 DNA Sequence (Notch1 + FLAG TAG)

ATGCCCCCCCTGCTGGCCCCCCTGCTGTGCCTGGCCCTGCTGCCTGCCCTGGCTGCCCGGGGCCCCCGGTGCTCCCA
GCCTGGCGAGACCTGCCTGAATGGCGGCAAGTGTGAGGCTGCCAATGGCACAGAGGCCTGTGTCTGTGGCGGCGCCT
TTGTGGGCCCCCGGTGCCAGGACCCCAACCCATGCCTGAGCACCCCATGCAAGAATGCTGGCACCTGCCATGTGGTG
GACCGGCGGGGCGTGGCTGACTATGCCTGCTCCTGTGCCCTGGGCTTCTCTGGCCCCCTGTGCCTGACCCCCCTGGA
CAATGCCTGCCTGACCAACCCATGCCGGAATGGCGGCACCTGTGACCTGCTGACCCTGACAGAGTACAAGTGCCGGT
GCCCCCCTGGCTGGTCTGGCAAGAGCTGCCAGCAGGCTGACCCATGTGCCTCCAACCCATGTGCCAATGGCGGCCAG
TGCCTGCCATTTGAGGCCAGCTACATCTGCCACTGCCCCCCATCCTTCCATGGCCCCACCTGCCGGCAGGATGTGAA
TGAGTGTGGCCAGAAGCCTGGCCTGTGCCGGCATGGCGGCACCTGCCACAATGAGGTGGGCAGCTACCGGTGTGTCT
GCCGGGCCACCCACACAGGCCCCAACTGTGAGCGGCCATATGTGCCATGCTCCCCAAGCCCATGCCAGAATGGCGGC
ACCTGCCGGCCCACAGGCGATGTGACCCATGAGTGTGCCTGCCTGCCTGGCTTCACAGGCCAGAACTGTGAGGAGAA
CATTGATGACTGCCCTGGCAACAACTGCAAGAATGGCGGCGCCTGTGTGGATGGCGTGAACACCTACAACTGCCGGT
GCCCCCCTGAGTGGACAGGCCAGTACTGCACAGAGGATGTGGATGAGTGCCAGCTGATGCCCAATGCCTGCCAGAAT
GGCGGCACCTGCCACAACACCCATGGCGGCTACAACTGTGTCTGTGTGAATGGCTGGACAGGCGAGGACTGCTCTGA
GAACATTGATGACTGTGCCTCTGCTGCCTGCTTCCATGGCGCCACCTGCCATGACCGGGTGGCCTCCTTCTACTGTG
AGTGCCCCCATGGCCGGACAGGCCTGCTGTGCCATCTGAATGATGCCTGCATCAGCAACCCATGCAATGAGGGCTCC
AACTGTGACACCAACCCTGTGAATGGCAAGGCCATCTGCACCTGCCCATCTGGCTACACAGGCCCTGCCTGCTCCCA
GGATGTGGATGAGTGCAGCCTGGGCGCCAACCCATGTGAGCATGCTGGCAAGTGCATCAACACCCTGGGCTCCTTTG
AGTGCCAGTGCCTGCAAGGCTACACAGGCCCCCGGTGTGAGATTGATGTGAATGAGTGTGTCAGCAACCCATGCCAG
AATGATGCCACCTGCCTGGACCAGATTGGCGAGTTCCAGTGCATCTGCATGCCTGGCTATGAGGGCGTGCACTGTGA
GGTGAACACAGATGAGTGTGCCTCCAGCCCATGCCTGCACAATGGCCGGTGCCTGGACAAGATCAATGAGTTCCAGT
GTGAGTGCCCCACAGGCTTCACAGGCCATCTGTGCCAGTATGATGTGGATGAGTGTGCCTCCACCCCATGCAAGAAT
GGCGCCAAGTGCCTGGATGGCCCCAACACCTACACCTGTGTCTGCACAGAGGGCTACACAGGCACCCACTGTGAGGT
GGACATTGATGAGTGTGACCCTGACCCATGCCACTATGGCAGCTGCAAGGATGGCGTGGCCACCTTCACCTGCCTGT
GCCGGCCTGGCTACACAGGCCATCACTGTGAGACCAACATCAATGAGTGCTCCAGCCAGCCATGCCGGCATGGCGGC
ACCTGCCAGGACCGGGACAATGCCTACCTGTGCTTCTGCCTGAAGGGCACCACAGGCCCCAACTGTGAGATCAACCT
GGATGACTGTGCCTCCTCCCCATGTGACTCTGGCACCTGCCTGGACAAGATTGATGGCTATGAGTGTGCCTGTGAGC
CTGGCTACACAGGCAGCATGTGCAACATCAACATTGATGAGTGTGCTGGCAACCCATGCCACAATGGCGGCACCTGT
GAGGATGGCATCAATGGCTTCACCTGCCGGTGCCCTGAGGGCTACCATGACCCCACCTGCCTGTCTGAGGTGAATGA
GTGCAACTCCAACCCATGTGTGCATGGCGCCTGCCGGGACAGCCTGAATGGCTACAAGTGTGACTGTGACCCTGGCT
GGTCTGGCACCAACTGTGACATCAACAACAATGAGTGTGAGTCCAACCCATGTGTGAATGGCGGCACCTGCAAGGAC
ATGACCTCTGGCTATGTCTGCACCTGCCGGGAGGGCTTCTCTGGCCCCAACTGCCAGACCAACATCAATGAGTGTGC
CAGCAACCCATGCCTGAACCAGGGCACCTGCATTGATGATGTGGCTGGCTACAAGTGCAACTGCCTGCTGCCATACA
CAGGCGCCACCTGTGAGGTGGTGCTGGCCCCATGTGCCCCATCCCCATGCCGGAATGGCGGCGAGTGCCGGCAGTCT
GAGGACTATGAGAGCTTCTCCTGTGTCTGCCCCACAGGCTGGCAGGCTGGCCAGACCTGTGAGGTGGACATCAATGA
GTGTGTGCTGAGCCCATGCCGGCATGGCGCCTCCTGCCAGAACACCCATGGCGGCTACCGGTGCCACTGCCAGGCTG
GCTACTCTGGCCGGAACTGTGAGACAGACATTGATGACTGCCGGCCCAACCCATGCCACAATGGCGGCTCCTGCACA
GATGGCATCAACACAGCCTTCTGTGACTGCCTGCCTGGCTTCCGGGGCACCTTCTGTGAGGAGGACATCAATGAGTG
TGCCTCTGACCCATGCCGGAATGGCGCCAACTGCACAGACTGTGTGGACAGCTACACCTGCACCTGCCCTGCTGGCT
TCTCTGGCATCCACTGTGAGAACAACACCCCTGACTGCACAGAGTCCAGCTGCTTCAATGGCGGCACCTGTGTGGAT
GGCATCAACTCCTTCACCTGCCTGTGCCCCCCTGGCTTCACAGGCAGCTACTGCCAGCATGATGTGAATGAGTGTGA
CTCCCAGCCATGCCTGCATGGCGGCACCTGCCAGGATGGCTGTGGCAGCTACCGGTGCACCTGCCCCCAGGGCTACA
CAGGCCCCAACTGCCAGAACCTGGTGCACTGGTGTGACTCCAGCCCATGCAAGAATGGCGGCAAGTGCTGGCAGACC
CACACCCAGTACCGGTGTGAGTGCCCATCTGGCTGGACAGGCCTGTACTGTGATGTGCCATCTGTCTCCTGTGAGGT
GGCTGCCCAGCGGCAGGGCGTGGATGTGGCCCGGCTGTGCCAGCATGGCGGCCTGTGTGTGGATGCTGGCAACACCC
ATCACTGCCGGTGCCAGGCTGGCTACACAGGCAGCTACTGTGAGGACCTGGTGGATGAGTGCTCCCCAAGCCCATGC
CAGAATGGCGCCACCTGCACAGACTACCTGGGCGGCTACTCCTGCAAGTGTGTGGCTGGCTACCATGGCGTGAACTG
CTCTGAGGAGATTGATGAGTGCCTGAGCCATCCATGCCAGAATGGCGGCACCTGCCTGGACCTGCCCAACACCTACA
AGTGCTCCTGCCCCCGGGGCACCCAGGGCGTGCACTGTGAGATCAATGTGGATGACTGCAACCCCCCTGTGGACCCT
GTCAGCCGGTCCCCCAAGTGCTTCAACAATGGCACCTGTGTGGACCAGGTGGGCGGCTACTCCTGCACCTGCCCCCC
TGGCTTTGTGGGCGAGCGGTGTGAGGGCGATGTGAATGAGTGCCTGAGCAACCCATGTGATGCCCGGGGCACCCAGA
ACTGTGTGCAGCGGGTGAATGACTTCCACTGTGAGTGCCGGGCTGGCCACACAGGCCGGCGGTGTGAGTCTGTGATC
AATGGCTGCAAGGGCAAGCCATGCAAGAATGGCGGCACCTGTGCTGTGGCCTCCAACACAGCCCGGGGCTTCATCTG
CAAGTGCCCTGCTGGCTTTGAGGGCGCCACCTGTGAGAATGATGCCCGGACCTGTGGCAGCCTGCGGTGCCTGAATG
GCGGCACCTGCATCTCTGGCCCCCGGTCCCCCACCTGCCTGTGCCTGGGCCCATTCACAGGCCCTGAGTGCCAGTTC
CCTGCCAGCTCCCCATGCCTGGGCGGCAACCCATGCTACAACCAGGGCACCTGTGAGCCCACCTCTGAGTCCCCATT
CTACCGGTGCCTGTGCCCTGCCAAGTTCAATGGCCTGCTGTGCCACATCCTGGACTACAGCTTTGGCGGCGGCGCTG
GCCGGGACATCCCCCCCCCCCTGATTGAGGAGGCCTGTGAGCTGCCTGAGTGCCAGGAGGATGCTGGCAACAAGGTC
TGCTCCCTCCAGTGCAACAACCATGCCTGTGGCTGGGATGGCGGCGACTGCTCCCTGAACTTCAATGACCCATGGAA
GAACTGCACCCAGAGCCTGCAATGCTGGAAGTACTTCTCTGATGGCCACTGTGACTCCCAGTGCAACTCTGCTGGCT
GCCTGTTTGATGGCTTTGACTGCCAGCGGGCTGAGGGCCAGTGCAACCCCCTGTATGACCAGTACTGCAAGGACCAC
TTCTCTGATGGCCACTGTGACCAGGGCTGCAACTCTGCTGAGTGTGAGTGGGATGGCCTGGACTGTGCTGAGCATGT
GCCTGAGCGGCTGGCTGCTGGCACCCTGGTGGTGGTGGTGCTGATGCCCCCTGAGCAGCTGCGGAACAGCTCCTTCC
ACTTCCTGCGGGAGCTGTCCCGGGTGCTGCACACCAATGTGGTCTTCAAGCGGGATGCCCATGGCCAGCAGATGATC
TTCCCATACTATGGCCGGGAGGAGGAGCTGCGGAAGCATCCCATCAAGCGGGCTGCTGAGGGCTGGGCTGCCCCTGA
TGCCCTGCTGGGCCAGGTGAAGGCCAGCCTGCTGCCTGGCGGCTCTGAGGGCGGCCGGCGGCGGCGGGAGCTGGACC
CCATGGATGTGCGGGGCTCCATTGTCTACCTGGAGATTGACAACCGGCAGTGTGTGCAGGCCAGCTCCCAGTGCTTC
CAGTCTGCCACAGATGTGGCTGCCTTCCTGGGCGCCCTGGCCAGCCTGGGCAGCCTGAACATCCCATACAAGATTGA
GGCTGTGCAGTCTGAGACAGTGGAGCCCCCCCCCCCTGCCCAGCTGCACTTCATGTATGTGGCTGCTGCTGCCTTTG
TGCTGCTGTTCTTTGTGGGCTGTGGCGTGCTGCTGTCCCGGAAGCGGCGGCGGCAGCATGGCCAGCTGTGGTTCCCT
GAGGGCTTCAAGGTCTCTGAGGCCTCCAAGAAGAAGCGGCGGGAGCCCCTGGGCGAGGACTCTGTGGGCCTGAAGCC
CCTGAAGAATGCCTCTGATGGCGCCCTGATGGATGACAACCAGAATGAGTGGGGCGATGAGGACCTGGAGACCAAGA
AGTTCCGGTTTGAGGAGCCTGTGGTGCTGCCTGACCTGGATGACCAGACAGACCATCGGCAGTGGACCCAGCAGCAT
CTGGATGCTGCTGACCTGCGGATGTCTGCCATGGCCCCCACCCCCCCCCAGGGCGAGGTGGATGCTGACTGCATGGA
TGTGAATGTGCGGGGCCCTGATGGCTTCACCCCCCTGATGATTGCCAGCTGCTCTGGCGGCGGCCTGGAGACAGGCA
ACTCTGAGGAGGAGGAGGATGCCCCTGCTGTGATCTCTGACTTCATCTACCAGGGCGCCTCCCTGCACAACCAGACA
GACCGGACAGGCGAGACAGCCCTGCATCTGGCTGCCCGGTACAGCCGGTCTGATGCTGCCAAGCGGCTGCTGGAGGC
CTCTGCTGATGCCAACATCCAGGACAACATGGGCCGGACCCCCCTGCATGCTGCTGTCTCTGCTGATGCCCAGGGCG
TCTTCCAGATCCTGATCCGGAACCGGGCCACAGACCTGGATGCCCGGATGCATGATGGCACCACCCCCCTGATCCTG
GCTGCCCGGCTGGCTGTGGAGGGCATGCTGGAGGACCTGATCAACTCCCATGCTGATGTGAATGCTGTGGATGACCT
GGGCAAGTCTGCCCTGCACTGGGCTGCTGCTGTGAACAATGTGGATGCTGCTGTGGTGCTGCTGAAGAATGGCGCCA
ACAAGGACATGCAGAACAACCGGGAGGAGACCCCCCTGTTCCTGGCTGCCCGGGAGGGCAGCTATGAGACAGCCAAG
GTGCTGCTGGACCACTTTGCCAACCGGGACATCACAGACCACATGGACCGGCTGCCCCGGGACATTGCCCAGGAGCG
GATGCATCATGACATTGTGCGGCTGCTGGATGAGTACAACCTGGTGCGGTCCCCCCAGCTGCATGGCGCCCCCCTGG
GCGGCACCCCCACCCTGTCCCCCCCCCTGTGCAGCCCCAATGGCTACCTGGGCTCCCTGAAGCCTGGCGTGCAGGGC
AAGAAGGTGCGGAAGCCAAGCTCCAAGGGCCTGGCCTGTGGCTCCAAGGAGGCCAAGGACCTGAAGGCCCGGCGGAA
GAAGTCCCAGGATGGCAAGGGCTGCCTGCTGGACTCCAGCGGCATGCTGTCCCCTGTGGACAGCCTGGAGTCCCCCC
ATGGCTACCTGTCTGATGTGGCCAGCCCCCCCCTGCTGCCATCCCCATTCCAGCAGTCCCCATCTGTGCCCCTGAAC
CATCTGCCTGGCATGCCTGACACCCATCTGGGCATTGGCCATCTGAATGTGGCTGCCAAGCCTGAGATGGCTGCCCT
GGGCGGCGGCGGCCGGCTGGCCTTTGAGACAGGCCCCCCCCGGCTGAGCCATCTGCCTGTGGCCTCTGGCACCTCCA
CAGTGCTGGGCAGCTCCTCTGGCGGCGCCCTGAACTTCACAGTGGGCGGCTCCACCAGCCTGAATGGCCAGTGTGAG
TGGCTGTCCCGGCTGCAATCTGGCATGGTGCCCAACCAGTACAACCCCCTGCGGGGCTCTGTGGCCCCTGGCCCCCT
GTCCACCCAGGCCCCAAGCCTGCAACATGGCATGGTGGGCCCCCTGCACTCCAGCCTGGCTGCCTCTGCCCTGTCCC
AGATGATGTCCTACCAGGGCCTGCCAAGCACCCGGCTGGCCACCCAGCCCCATCTGGTGCAGACCCAGCAGGTGCAG
CCCCAGAACCTGCAAATGCAGCAGCAGAACCTGCAACCTGCCAACATCCAGCAGCAGCAGTCCCTCCAGCCCCCCCC
CCCCCCCCCCCAGCCCCATCTGGGCGTCAGCTCTGCTGCCTCTGGCCATCTGGGCCGGTCCTTCCTGTCTGGCGAGC
CAAGCCAGGCTGATGTGCAGCCCCTGGGCCCATCCTCCCTGGCTGTGCACACCATCCTGCCCCAGGAGAGCCCTGCC
CTGCCCACCTCCCTGCCAAGCTCCCTGGTGCCCCCTGTGACAGCTGCCCAGTTCCTGACCCCCCCATCCCAGCACTC
CTACTCCAGCCCTGTGGACAACACCCCATCCCATCAGCTCCAGGTGCCTGAGCATCCATTCCTGACCCCAAGCCCTG
AGTCCCCTGACCAGTGGTCCAGCTCCTCCCCCCACAGCAATGTCTCTGACTGGTCTGAGGGCGTCTCCTCCCCCCCC
ACCAGCATGCAGTCCCAGATTGCCAGAATCCCTGAGGCCTTCAAGGATTACAAGGACGACGATGACAAGTAA
(SEQ ID NO: 1)

APPENDIX II
Notch1 Protein Sequence (Notch1 + FLAG TAG)

MPPLLAPLLCLALLPALAARGPRCSQPGETCLNGGKCEAANGTEACVCGGAFVGPRCQDPNPCLSTPCKNAGTCHVV
DRRGVADYACSCALGFSGPLCLTPLDNACLTNPCRNGGTCDLLTLTEYKCRCPPGWSGKSCQQADPCASNPCANGGQ
CLPFEASYICHCPPSFHGPTCRQDVNECGQKPGLCRHGGTCHNEVGSYRCVCRATHTGPNCERPYVPCSPSPCQNGG
TCRPTGDVTHECACLPGFTGQNCEENIDDCPGNNCKNGGACVDGVNTYNCRCPPEWTGQYCTEDVDECQLMPNACQN
GGTCHNTHGGYNCVCVNGWTGEDCSENIDDCASAACFHGATCHDRVASFYCECPHGRTGLLCHLNDACISNPCNEGS
NCDTNPVNGKAICTCPSGYTGPACSQDVDECSLGANPCEHAGKCINTLGSFECQCLQGYTGPRCEIDVNECVSNPCQ
NDATCLDQIGEFQCICMPGYEGVHCEVNTDECASSPCLHNGRCLDKINEFQCECPTGFTGHLCQYDVDECASTPCKN
GAKCLDGPNTYTCVCTEGYTGTHCEVDIDECDPDPCHYGSCKDGVATFTCLCRPGYTGHHCETNINECSSQPCRHGG
TCQDRDNAYLCFCLKGTTGPNCEINLDDCASSPCDSGTCLDKIDGYECACEPGYTGSMCNINIDECAGNPCHNGGTC
EDGINGFTCRCPEGYHDPTCLSEVNECNSNPCVHGACRDSLNGYKCDCDPGWSGTNCDINNNECESNPCVNGGTCKD
MTSGYVCTCREGFSGPNCQTNINECASNPCLNQGTCIDDVAGYKCNCLLPYTGATCEVVLAPCAPSPCRNGGECRQS
EDYESFSCVCPTGWQAGQTCEVDINECVLSPCRHGASCQNTHGGYRCHCQAGYSGRNCETDIDDCRPNPCHNGGSCT
DGINTAFCDCLPGFRGTFCEEDINECASDPCRNGANCTDCVDSYTCTCPAGFSGIHCENNTPDCTESSCFNGGTCVD
GINSFTCLCPPGFTGSYCQHDVNECDSQPCLHGGTCQDGCGSYRCTCPQGYTGPNCQNLVHWCDSSPCKNGGKCWQT
HTQYRCECPSGWTGLYCDVPSVSCEVAAQRQGVDVARLCQHGGLCVDAGNTHHCRCQAGYTGSYCEDLVDECSPSPC
QNGATCTDYLGGYSCKCVAGYHGVNCSEEIDECLSHPCQNGGTCLDLPNTYKCSCPRGTQGVHCEINVDDCNPPVDP
VSRSPKCFNNGTCVDQVGGYSCTCPPGFVGERCEGDVNECLSNPCDARGTQNCVQRVNDFHCECRAGHTGRRCESVI
NGCKGKPCKNGGTCAVASNTARGFICKCPAGFEGATCENDARTCGSLRCLNGGTCISGPRSPTCLCLGPFTGPECQF
PASSPCLGGNPCYNQGTCEPTSESPFYRCLCPAKFNGLLCHILDYSFGGGAGRDIPPPLIEEACELPECQEDAGNKV
CSLQCNNHACGWDGGDCSLNFNDPWKNCTQSLQCWKYFSDGHCDSQCNSAGCLFDGFDCQRAEGQCNPLYDQYCKDH
FSDGHCDQGCNSAECEWDGLDCAEHVPERLAAGTLVVVVLMPPEQLRNSSFHFLRELSRVLHTNVVFKRDAHGQQMI
FPYYGREEELRKHPIKRAAEGWAAPDALLGQVKASLLPGGSEGGRRRRELDPMDVRGSIVYLEIDNRQCVQASSQCF
QSATDVAAFLGALASLGSLNIPYKIEAVQSETVEPPPPAQLHFMYVAAAAFVLLFFVGCGVLLSRKRRRQHGQLWFP
EGFKVSEASKKKRREPLGEDSVGLKPLKNASDGALMDDNQNEWGDEDLETKKFRFEEPVVLPDLDDQTDHRQWTQQH
LDAADLRMSAMAPTPPQGEVDADCMDVNVRGPDGFTPLMIASCSGGGLETGNSEEEEDAPAVISDFIYQGASLHNQT
DRTGETALHLAARYSRSDAAKRLLEASADANIQDNMGRTPLHAAVSADAQGVFQILIRNRATDLDARMHDGTTPLIL
AARLAVEGMLEDLINSHADVNAVDDLGKSALHWAAAVNNVDAAVVLLKNGANKDMQNNREETPLFLAAREGSYETAK
VLLDHFANRDITDHMDRLPRDIAQERMHHDIVRLLDEYNLVRSPQLHGAPLGGTPTLSPPLCSPNGYLGSLKPGVQG
KKVRKPSSKGLACGSKEAKDLKARRKKSQDGKGCLLDSSGMLSPVDSLESPHGYLSDVASPPLLPSPFQQSPSVPLN
HLPGMPDTHLGIGHLNVAAKPEMAALGGGGRLAFETGPPRLSHLPVASGTSTVLGSSSGGALNFTVGGSTSLNGQCE
WLSRLQSGMVPNQYNPLRGSVAPGPLSTQAPSLQHGMVGPLHSSLAASALSQMMSYQGLPSTRLATQPHLVQTQQVQ
PQNLQMQQQNLQPANIQQQQSLQPPPPPPQPHLGVSSAASGHLGRSFLSGEPSQADVQPLGPSSLAVHTILPQESPA
LPTSLPSSLVPPVTAAQFLTPPSQHSYSSPVDNTPSHQLQVPEHPFLTPSPESPDQWSSSSPHSNVSDWSEGVSSPP
TSMQSQIARIPEAFKDYKDDDDK (SEQ ID NO: 2)

APPENDIX III
Group I - Notch1 specific antibodies

A. Aminoacid sequence
A.1- Binding to LBD (N1-EGF 1-13)

Heavy Chain

| Clone Name | HC-sequences |
|---|---|
| hN1S3- | EVQLVQSGAEVKKPGASVKVSCKASGYNFTGYYMHWVRQAPGQGLEWMGWIN |
| 009 | PNSGGTNYAQKFQGRVTMTRDTSISTAYMELSRLRSDDTAVYYCATDSFDYWGQ |
| | GTMVTVSS (SEQ ID NO: 3) |
| hN1S3- | EVQLVQSGAEVKKPGASVKVSCKASGYNFTGYYMHWVRQAPGQGLEWMGWIN |
| 010 | PNSGGTNYAQKFQGRVIMTRDTSISTAYMELSRLTSDDTAVYYCATDSFDYWGRG |
| | TMVTVSS (SEQ ID NO: 4) |
| hN1S3- | QVQLVQSGPEVKKPGASVKVSCKASGYNFTGYYMHWVRQAPGQGLEWMGWIN |
| 011 | PNSGGTNYAQKFQGRVTMTRDTSISTAYMELSRLRSDDTAVYYCATDSFDYWGR |
| | GTMVTVSS (SEQ ID NO: 5) |
| hN1S3- | EVQLVQSGAEVKKPGASVKVSCKASGYNFTGYYMHWVRQAPGQGLEWMGWIN |
| 013 | PNSGGTNYAQKFQGRVTMTRDTSISTAYMELSRLRSDDTAVYYCATDSFDYWGR |
| | GTLVTVSS (SEQ ID NO: 6) |
| hN1S3- | QVQLVQSGAEVKKPGASVKVSCKASGYNFTGYYMHWVRQAPGQGLEWMGWIN |
| 050 | PNSGGTNYAQKFQGRVTMTRDTSISTAYMELSRLRSDDTAVYYCATDSFDYWGR |
| | GTMVTVSS (SEQ ID NO: 7) |
| hN1S3- | QVQLVQSGAEVKKPGASVRISCKTSGFTFTSYFIHWVRQAPGQRPEWMGVINPSRGN |
| 052 | ADYAPRFRGRVTMTRDKSTHTVYMDLKSLRSDDAAIYYCARDRLGGLLDYYDSSDSSK |
| | NFDAFDFWGQGTLVTVSS (SEQ ID NO: 8) |
| hN1S3- | EVQLVQSGAEVKKPGASVKVSCKASGYNFTGYYMHWVRQAPGQGLEWMGWIN |
| 094 | PNSGGTNYAQKFQGRVTMTRDTSISTAYMELSRLRSDDTAVYYCATDSFDYWGR |
| | GTMVTVSS (SEQ ID NO: 9) |
| hN1S3- | EVQLVQSGAEVKKPGASVKVSCKASGYNFTGYYMHWVRQAPGQGLEWMGWIN |
| 113 | PNSGGTNYAQKFQGRVTMTRDTSISTAYMELSRLRSDDTAVYYCATDSFDYWGR |
| | GTLVTVSS (SEQ ID NO: 10) |
| hN1S3- | EVQLVQSGAEVKKPGASVKVSCKASGYNFTGYYMHWVRQAPGQGLEWMGWIN |
| 126 | PNSGGTNYAQKFQGRVTMTRDTSISTAYMELSRLRSDDTAVYYCATDSFDYWGQ |
| | GTLVTVSS (SEQ ID NO: 11) |
| hN1S3- | EVQLVQSGPEVKKPGTSVKVSCKASGFTFTSSAVQWVRQARGQRLEWIGWIVVG |
| 146 | SGNTNYAQKFQERVTITRDMSTSTAYMELSSLRSEDTAVYYCVADTVYWGKGTLV |
| | TVSS (SEQ ID NO: 12) |
| hN1S3- | EVQLVQSGAEVKKPGASVKVSCKASGYNFTGYYMHWVRQAPGQGLEWMGWIN |
| 174 | PNSGGTNYAQKFQGRVTMTRDTSISTAYMELSRLRSDDTAVYYCATDSFDYWGQ |
| | GTTVTVSS (SEQ ID NO: 13) |
| hN1S3- | QVQLQQSGAEVKKPGASVKVSCKASGYNFTGYYMHWVRQAPGQGLEWMGWIN |
| 056 | PNSGGTNYAQKFQGRVTMTRDTSISTAYMELSRLRSDDTAVYYCATDSFDYWGR |
| | GTLVTVSS (SEQ ID NO: 14) |
| N1wc613 | QVQLVQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGR |
| | GTLVTVSS (SEQ ID NO: 15) |
| N1wc133 | QVQLVQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGQ |
| | GTLVTVSS (SEQ ID NO: 16) |
| N1wc173 | EVQLVQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGQ |
| | GTTVTVSS (SEQ ID NO: 17) |
| N1wc155 | EVQLVQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGR |
| | GTLVTVSS (SEQ ID NO: 18) |
| N1wc168 | EVQLVQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGR |
| | GTLVTVSS (SEQ ID NO: 19) |
| N1wc159 | QLQLVETGGGLVKPGESLTLSCAASGFTFSSYSMNWVRQAPGKGLEWVSSISSS |
| | GSSIYYADSLKGRFTISRDNAKNSLYLQMNSLRAEDTAVYYCTRGTQWLAGVGDY |
| | WGKGTLVTVSS (SEQ ID NO: 20) |
| N1wc97 | EVQLVQSGAEVKKPGASVKVSCKASGYNFTGYYMHWVRQAPGQGLEWMGWIN |
| | PNSGGTNYAQKFQGRVAMTRDTSISTAYMELSRLRSDDSAVYYCATDSFDYWGR |
| | GTTVTVSS (SEQ ID NO: 21) |
| N1wc49 | EVQLVESGAEVKKPGASLNLSCKASGYSFNKYYIHWVRQAPGQGLELMGRINPGS |
| | GTTSYAQKFQGRVTLSRDTSTNTIFMELNGLTGEDTAIYYCARSNYDILAGYGPDA |
| | FDIWGRGTLVTVSS (SEQ ID NO: 22) |

Light Chain

| Clone Name | LC amino acid sequences |
|---|---|
| hN1S3- | SYVLTQPPSVSEAPRQRVTISCSGSSSNIGNNAVSWYQQLPGKAPKTLIYYDDLLS |
| 009 | SGVSDRFSGSRSGTSASLAISGLQSEDEADYYCAAWDDSLNGVVFGGGTKLTVL |
| | (SEQ ID NO: 23) |
| hN1S3- | QSVLTQPPSASGTPGQRVTISCSGSNSNIGSNYVFWYQQLPGTAPKLLIYNNNQR |
| 010 | PSGVPDRFSGSKSGTSASLAISGLRSEDEADYYCAAWDDSLSGVVFGGGTKLTVL |
| | (SEQ ID NO: 24) |
| hN1S3- | QSVLTQPPSASGTPGQRVTISCSGSSSNIGSNTVNWYQQLPGTAPKLLIYSNNQR |
| 011 | PSGVPDRFSGSKSGTSASLAISGLQSEDEADYYCAAWDDSLNGVVFGGGTKLTVL |
| | (SEQ ID NO: 25) |
| hN1S3- | QPVLTQPPSASGTPGQRVTISCSGSSSNIGSNYVYWYQKFPGTAPKLLIYTNNQR |
| 013 | PSGVPDRFSGSKSGTSASLAISGLRSEDEADYYCAAWDDSLSGVVFGGGTKVTVL |
| | (SEQ ID NO: 26) |
| hN1S3- | LPVLTQPPSASGTPGQRVTISCSGRSSNIGSNFVYWYQQLPGTAPKLLIYRNNQRP |
| 050 | SGVPDRFSGSKSGTSASLAISGLRSEDEADYYCATWDDNLSGVVFGGGTKLTVL |
| | (SEQ ID NO: 27) |
| hN1S3- | QAVLTQPSLASGTPGQRVTISCSGGSSNIGRHTVNWYQQLPGAAPKLLMHSSDQ |
| 052 | RPSGVPDRFSGSKSGTSASLAISGLQSEDEADYYCAAWDASLKNWVFGGGTKLT |
| | VL (SEQ ID NO: 28) |
| hN1S3- | QSVLTQPPSASGTPGQRVTISCSGGSSNIGSNTVTWYQQLPGKAPKLLIYISDQRP |
| 094 | SGVPDRFSGSKSGTSASLAIRGLQSEDEADYYCAAWDDSLNGAVFGTGTKVTVL |
| | (SEQ ID NO: 29) |
| hN1S3- | SYVLTQPPSASGTPGQRVTISCSGSSPNIGSNTVNWYQQLPGTAPKLLIYTNNQRP |
| 113 | SGVPDRFSGSKSATSASLAISGLQFDDEADYFCAAWDDSLLGWFGGGTKVTVL |
| | (SEQ ID NO: 30) |
| hN1S3- | SYVLTQPPSASGAPGQRVTISCSGSISNIGDNTVNWYQHLPRTAPKLVIYSDVQRP |
| 126 | SGVPERFSGSKSGTSASLVISGLQAEDEADYYCASWDDSLDGVFFGGGTKLTVL |
| | (SEQ ID NO: 31) |
| hN1S3- | QAVLTQPSSVSEAPRQRVTISCSGSSSNIGKNAVNWYQQLPGKAPKLLIYYDDLM |
| 146 | PSGVSDRFSGSKSGTSASLAISGLQSEDEADYFCAAWDDSLSGWVFGGGTKLTV |
| | L (SEQ ID NO: 32) |
| hN1S3- | QSVLTQPPSVSGAPGQRITISCTGSSSNIGAGYDVHWYQQLPGTAPKLLIYGNSYR |
| 174 | PSGVPDRFSGSKSGTSASLAITGLQAEDEADYYCQSYDSSLTGSIFGGGTKLTVL |
| | (SEQ ID NO: 33) |
| hN1S3- | QSVLTQPPSASGTPGQRVTISCSGSSSNIGSNTVNWYQQLPGTAPKLLIYSNNQR |
| 056 | PSGVPDRFSGSKSGTSASLAISGLQSEDEADYYCAAWDDSLNGLVFGGGTKVTVL |
| | (SEQ ID NO: 34) |
| N1wc613 | SYELTQPPSASGTPGQRVTISCSGSYSNIGSNYVYWYQQLPGTAPKLLIYTNNQRP |
| | SGVPDRFSGSKSGTSASLVISGLRSDDEGDYYCAAWDDSVSVLLFGGGTKVTVL |
| | (SEQ ID NO: 35) |
| N1wc133 | SYELTQPPSASGTPGQRVTISCSGSNSNIGSNTLNWYQQLPGTAPKLLIYSNNQRP |
| | SWVPDRFSGSKSGTSASLAISGLQSEDEADYYCAAWDDSLNGLIFGGGTKLTVL |
| | (SEQ ID NO: 36) |
| N1wc173 | QPVLTQPPSASWTPGQRVTISCSGSSSNIGSNTVHWYRQFPGTAPQLLIYSNNQR |
| | PSGVPDRFSGSKSGTSASLAIRGLQSEDEADYYCAAWDDSLNGVVFGGGIKLTVL |
| | (SEQ ID NO: 37) |
| N1wc155 | QSVLTQPPSVSGAPGQRVTISCTGSSSNIGAGYDVLWYRQLPGTAPKLLIYGNTH |
| | RPSGVPDRFSGSNSGTSASLAITGLQAEDEADYYCQSYDSSLSGSVFGGGTKLTV |
| | L (SEQ ID NO: 38) |
| N1wc168 | QSVLTQPPSASGTPGQRVTISCSGSSSNIGSNIVNWYQQLPGTAPKLLIYSNNQRP |
| | SGVPDRFSGSKSGTSASLAISGLQSEDEADYYCAAWDDSLNGVVFGGGTKLTVL |
| | (SEQ ID NO: 39) |
| N1wc159 | QSALTQPPSASASPGQSVTISCTGTSSDVGAYDSVSWYQQHPGKAPKLIIYDVNN |
| | RPSGVSNRFSGSESGNTASLTISGLQAEDEANYYCSSYRNTKTVVFGGGTKLTVL |
| | (SEQ ID NO: 40) |
| N1wc97 | QSVLTQPPSVSGAPGQRVTISCTGTSSNIGAGYDVQWYQQLPGTAPKVLMYGNS |
| | NRPSGVPDRFSGSKSGTSASLAITGLQAEDEADYYCQSYDSSLSGSLFGTGTKVTVL |
| | (SEQ ID NO: 41) |
| N1wc49 | QSVVTQPPSVSGAPGQTVTISCTGNSSNIGSSVDVHWYQQLPGTAPKLLIYDNKN |
| | RPSGVPDRFSGSKSGTSASLAITGLQAEDEADYYCQSYDSSLSSEVFGGGTKVTVL |
| | (SEQ ID NO: 42) |

A.2- Binding to NRR
Heavy Chain

| Clone Name | HC-sequences |
|---|---|
| N1wc629 | EVQLVQSGAEVKKPGSSVKVSCKASGGTLSSYTVSWLRQAPGQGLEWMGRIIPIL |
| | DRANYAQKFQGRVTITADKSTSTAYMELNSLRSDDTAVYYCARSIGAAGDGVWFD |
| | PWGQGTMVTVSS (SEQ ID NO: 43) |
| N1wc75 | GVQLVQSGGGLVQPGGSLRLSCAASGFTFDDYAMHWVRQAPGKGLEWVSSISW |
| | HSRTIAYADSVKGRFSISRDNAKNSLYLQMNSLRPEDTAVYYCAKASYLSTSSSLD |
| | YWGRGTLVTVSS (SEQ ID NO: 44) |
| N1wc104 | QVQLQESGPGLVKPSGTLSLTCAVSGVSLATNNWWLWVRQSPGKGLEWIGEIYH |
| | SGYTNYNPSLKSRVTISVDKSSNQLSLDLRSITAADTAVYYCARRYCDNGVCYPFD |
| | HWGQGTMVTVSS (SEQ ID NO: 45) |

Light Chain

| Clone Name | LC amino acid sequences |
|---|---|
| N1wc629 | QAVLTQPSSVSGAPGQRVTISCTGSSSNIGAGYDVHWYQQLPGTAPKLLIFDNKN |
| | RPSGVPDRFSGSNSGTSASLAITGLQAEDEAEYYCQSYDNNLSGRVFGGGTKLTVL |
| | (SEQ ID NO: 46) |
| N1wc75 | QSVLTQPGSVSGSPGQSITISCTGTSSDVGGYNYVSWYQQHPGKAPKLMIYEGSK |
| | RPSGVSNRFSGSKSGNTASLTISGLQAEDEADYYCSSYTTRSTRVFGGGTKLTVL |
| | (SEQ ID NO: 47) |
| N1wc104 | SYVLTQPPSVSEAPRQTVTISCSGNSFNIGRYPVNWYQQLPGKAPKLLIYYNNLRF |
| | SGVSDRFSGSKSGTSASLAIRDLLSEDEADYYCSTWDDTLKGWVFGGGTKLTVL |
| | (SEQ ID NO: 48) |

A.3- Additional Notch Antibodies
Heavy Chain

| Clone Name | HC-sequences |
|---|---|
| hN1S3- | EVQLVQSGAEVKKPGASVRISCKTSGFTFTSYFIHWVRQAPGQRPEWMGVINPSR |
| 067 | GNADYAPRFRGRVTMTRDKSTHTVYMDLKSLRSDDAAIYYCARDRLGGLLDYYDS |
| | SDSSKNFDAFDFWGQGTLVTVSS (SEQ ID NO: 49) |
| hN1S3- | EVQLVQSGAEVKKPGASVKVSCKASGYTFTDYYMHWVRQAPGQGLEWMGWINP |
| 116 | NSGGTYFPQNFQGRVTMTSDPSISAAYMELSNLRSDDTAVYFCARGAYDYVWGS |
| | HRYSGDAFDFWGQGTLVTVSS (SEQ ID NO: 50) |
| hN1S3- | QVQLVQSGAEVKKPGASVKVSCKASGYNFTGYYMHWVRQAPGQGLEWMGWIN |
| 193 | PNSGGTNYAQKFQGRVTMTRDTSISTAYMELSRLRSDDTAVYYCATDSFDYWGQ |
| | GTLVTVSS (SEQ ID NO: 51) |
| hN1S3- | QVQLVQSGAEVKKPGASVRISCKTSGFTFTSYFIHWVRQAPGQRPEWMGVINPS |
| 073 | RGNADYAPRFRGRVTMTRDKSTHTVYMDLKSLRSDDAAIYYCARDRLGGLLDYY |
| | DSSDSSKNFDAFDFWGQGTLVTVSS (SEQ ID NO: 52) |
| N1wc14 | EVQLVQSGGGLVQPGRSLRLSCAASGFTSDDYAMHWVRQAPGKGLD*VTAITWN |
| | SGHKDYADSVKGRFAVSRDNAKNALYLQMNSLRPEDTAVYYCAKASYLSTSSSLD |
| | YWGRGTMVTVSS (SEQ ID NO: 53) |
| N1wc23 | EVQLVQSGAEVKKPGASVKVSCKASGYNFTGYYIHWVRQAPGQGLEWMGWINP |
| | NSGGTNYAQKFQGRVIMTRDTSISTAYMELSRLRSDDTAVYYCATDSFDYWGRG |
| | TLVTVSS (SEQ ID NO: 54) |
| N1wc33 | EVQLVESGGDLVQPGGSLRLSCAASGFTFSDSWMHWVRQVPGKGLEWVSHIGID |
| | GITTNYADSVKGRFTISRDNAKNTLYLQMNNLRAEDTAVYYCARNVGWESRPWG |
| | QGTLVTVSS (SEQ ID NO: 55) |
| N1wc48 | QVQLVQSGAEVKKPGATVKISCKVSGYTFTDYYIHWVQQAPGKGLEWMGLVDPE |
| | DGETIYAEKFQGRVTIIADTSTDTAYMELSSLRSEDTAVYYCATGPFRLDSWGQGT |
| | MVTVSS (SEQ ID NO: 56) |
| N1wc60 | QVQLVQSGAEVKKPGASVKVSCKASGYNFTGYYMHWVRQAPGQGLEWMGWIN |
| | PNSGGTNYAQKFQGRVTMTRETSISTAYMELSRLRSDDTAVYYCATDSFDYWGR |
| | GTLVTISS (SEQ ID NO: 57) |
| N1wc99 | QVQLVQSGAEVKKPGASVKVSCKASGYTFTSYGISWVRQAPGQGLEWMGWISA |
| | YNGNTNYAQKLQGRVTMTTDTSTSTAYMELRSLRSDDTAVYYCARLASRAFDIWG |
| | RGTLVTVSS (SEQ ID NO: 58) |
| N1wc103 | QVQLQQSGAEVKKPGASVKVSCKASGYNFTGYYMHWVRQAPGQGLEWMGWIN |
| | PNSGGTNYAQKFQGRVTMTRDTSISTAYVELSRLRSDDTAVYYCATDSFDYWGR |
| | GTLVTVSS (SEQ ID NO: 59) |
| N1wc106 | QVQLVQSGAEVKRPGASVNLSCKASGYSFSRYYIHWVRQAPGQGLELMGRINPG |
| | GGTTTYAQKFQGRVTLSRDTSTNTIFMELNGLTSEDTAMYYCARSNYDILAGYGP |
| | DAFDFWGRGTLVTVSS (SEQ ID NO: 60) |
| N1wc110 | EVQLVQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGQ |
| | GTMVTVSS (SEQ ID NO: 61) |
| N1wc112 | EVQLVESGGGLAQPGKSLRISCAASGFTFGDYAMTWVRQAPGKGLEWVSSISGS |
| | GSGTYYTDSVKGRFAISRDNSKNTLYPQMNSLAAEDTAIYFCAKDQTDVTIRDFDY |
| | WGRGTLVTVSS (SEQ ID NO: 62) |
| N1wc137 | QVQLVQSGAEVKKPGASVKVSCKASGYTFTNYGLSWVRQAPGQGLEWMGWISG |
| | YNDNTSYAQKFQDRITMTTDTSTSTASMELRSLRSDDTAVYYCAFSYYDSSGYFN |
| | WGQGTMVTVSS (SEQ ID NO: 63) |
| N1wc140 | QVQLQQSGAEVKKPGSSVKVSCRASGGTFSNYAISWVRQAPGQGLEWMGRIIPIF |
| | GGAHYAQKLQGRVSITADESTSTAHMELSSLRSEDAAVYYCASGAYYDVMGNYP |
| | YSGMDVWGRGTLVTVSS (SEQ ID NO: 64) |
| N1wc142 | EVQLVQSGAEVKKPGASVNLSCKASGYSFSRYYIHWVRQAPGQGLELMGRINPG |
| | GGTTTYAQKFQGRVTLSRDTSTNTIFMELNGLTSEDTAMYYCARSNYDILAGYGP |
| | DAFDFWGRGTMVTVSS (SEQ ID NO: 65) |
| N1wc145 | EVQLVESGAEVKKPGASVKVSCKASGYTFTTYGISWVRQAPGQGLEWMGWISTY |
| | NGNTKYAQKLQGRVTMTTDTSTSTAYMELRSLRSDDTAVYYCARDARKAFDIWG |
| | RGTLVTVSS (SEQ ID NO: 66) |
| N1wc152 | QVQLVQSGGGLVQPGGSLRLSCGASGFTFSSHWMSWVRRPPGRGLEWVANINE |
| | GGSAEYYADSLKGRFTISRDNAKNSLFLQMDSLRAEDTAVYYCVRDQFHSNYDW |
| | GQGTMVTVSS (SEQ ID NO: 67) |
| N1wc157 | QVQLVQSGAGLKRPGTSVKISCKTSGYIFSQYPMHWVRQAPGQGLEWVAWVDT |
| | GNGTTRYSPNFQGRATVSGDTSANTGYLELRSLRFTDTAVYYCATNAFDYWGRG |
| | TLVTVSS (SEQ ID NO: 68) |
| N1wc158 | EVQLVQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGR |
| | GTLVTVSS (SEQ ID NO: 69) |
| N1wc160 | QVQLQESGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGR |
| | GTMVTVSS (SEQ ID NO: 70) |
| N1wc175 | QVQLVQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGR |
| | GTLVTVSS (SEQ ID NO: 71) |
| N1wc179 | EVQLVQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGQ |
| | GTMVTVSS (SEQ ID NO: 72) |
| N1wc191 | QVQLVQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGQ |
| | GTMVTVSS (SEQ ID NO: 73) |
| N1wc202 | EVQLVQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGR |
| | GTLVTVSS (SEQ ID NO: 74) |
| N1wc520 | QVQLVQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGR |
| | GTTVTVSS (SEQ ID NO: 75) |
| N1wc625 | QVQLVQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGQ |
| | GTMVTVSS (SEQ ID NO: 76) |

Light Chain

| Clone Name | LC amino acid sequences |
|---|---|
| hN1S3- | NFMLTQPHSVSESPGKTVIISCTRSSGSIASNYVQWLQQRPGSSPTTVIYDDNRRP |
| 067 | SGVPDRFSGSIDGSSNSASLTISGLKTEDEADYYCQSYDRFDHVVFGGGTKLTVL |
| | (SEQ ID NO: 77) |
| hN1S3- | QSVLTQPPSASGTPGQRVTISCSGSSSNIGSNTVNWYQRLPGAAPQLLIYNNDQR |
| 116 | PSGIPDRFSGSKSGTSGSLVISGLQSEDEADYYCASWDDSLNGRVFGGGTKLTVL |
| | (SEQ ID NO: 78) |
| hN1S3- | SYELTQPPSASGTPGQRVTISCSGSSSNIGSNTVNWYQQLPGTAPKLLIYSNNQR |
| 193 | PSGVPDRFSGSKSGTSASLAISGLQSEDEADYYCAAWDDSLNGYVFGTGTKLTVL |
| | (SEQ ID NO: 79) |
| hN1S3- | DIQMTQSPSSLSAFVGDRVTITCRASQGINNYLAWFQQKPGKAPKSLIYAASTLRS |
| 073 | GVPPRFSGSGSGTDFTLTISSLQPEDFATYYCQQYDSYPHTFGQGTRLEIK (SEQ |
| | ID NO: 80) |
| N1wc14 | SYVLTQPPSVSVAPGQTARITCGGNNIGSKSVHWYQQKPGQAPVLVIYEDSKRPS |
| | GIPERFSGSSSGTMATLTISGAQVEDEADYYCYSTDSSGNHRVFGTGTKVTVL |
| | (SEQ ID NO: 81) |
| N1wc23 | QSVLTQPPSASGTPGQRVTISCSGSYSNIGSNYVYWYQQFPGTAPKLLIYTNNQR |
| | PSGVPDRFSGSKSGSSASLAISGLRSEDEADYYCAAWDDSVGVLLFGGGTKLTVL |
| | (SEQ ID NO: 82) |
| N1wc33 | SYVLTQPPSASGAPGQRVTISCSGSDSNIGRNTVNWYQQQLPGTAPTLLIYTNNQ |
| | RPSGVPDRFSGSKSGTSASLAISGLQSGDEADYYCAAWDDSLNAYFFGPGTKLTVL |
| | (SEQ ID NO: 83) |
| N1wc48 | QSVLTQPPSVSGAPGQRVTISCTGSSSNIGAGYDVHWYQQLPGSAPKLLIYGNNN |
| | RPSGVPDRFSGSKSGTSASLAITGLQAEDEADYYCQSFDNRLSDFNVFGTGTKVTVL |
| | (SEQ ID NO: 84) |
| N1wc60 | QSVLTQPPSASGTPGQRVTISCSGSSSNIGSNYVYWYQQLPGTAPKLLIYRNNQR |
| | PSGVPDRFSGSKSGTSASLAISGLRSEDEADYYCAAWDDSLSGLVFGGGTKLTVL |
| | (SEQ ID NO: 85) |
| N1wc99 | QSVLTQPASVSGSPGQSITISCTGTSSDVGGYNYVSWYQQHPGKAPKLMIYEGSK |
| | RPSGVSNRFSGSKSGNTASLTISGLQAEDEADYYCSSYTTRSTRVFGGGTKLTVL |
| | (SEQ ID NO: 86) |
| N1wc103 | QSVLTQPPSASGTPGQRVTISCSGSSSNIGSNPVNWYQQLPGTAPKLLIYSNNQR |
| | PSGVPDRFSGSKSGTSASLAISGLHSEDEADYYCAAWDDSLNGSSVFGTGTKLTVL |
| | (SEQ ID NO: 87) |
| N1wc106 | QAVLTQPSSVSGAPGQRVTISCTGSSSNIGADNDVHWYQKFPGTAPKLLIFSNNN |
| | RPSGVPDRFSGSKSGTSASLAITGLQADDEADYYCQSYDSGLTRELFGGGTKLTVL |
| | (SEQ ID NO: 88) |
| N1wc110 | QSVVTQPPSASGSPGQRVTIACSGSSSNIGSNTVNWYQHVPGTAPKLLIYNNNQR |
| | PSGVQDRFSGSKSDTSASLAISGLQAEDEAVYYCATWDDSLIGLAFGGGTKLTVL |
| | (SEQ ID NO: 89) |
| N1wc112 | SYELTQPPSVSVAPGQTARISCGGDNLGRKSVHWYQQKPGQAPVLVIYEDSKRP |
| | SGIPERFSGSSSGTMATLTISGAQVEDEADYYCYSTDSSGNHRVFGGGTKVTVL |
| | (SEQ ID NO: 90) |
| N1wc137 | QAVLTQPSSVSGAPGQRVTVSCTGSSSNIGTGYAVHWYQQLPGTAPRLLIFGNNN |
| | RPSGVPDRFSGSKSGTSASLVITGLQAEDEADYYCQSFDTLSGSRVFGGGTKVTVL |
| | (SEQ ID NO: 91) |
| N1wc140 | SYVLTQPPSVSVSPGQTAKITCGGDKIETKSVHWYQQKPGQAPVLVIYEDTKRPS |
| | GISERLSGSSAGTVATLTITGAQVDDEADYFCYSTDATGTERVFGGGTKLTVL |
| | (SEQ ID NO: 92) |
| N1wc142 | QSVLTQPPSVSGAPGQRVTISCIGSNSNIGAGHDVHWYQQFPGTAPKLLIYDNTN |
| | RPSGVPDRFSGSKSGTSVSLAITGLQAEDEADYYCQSYDSSLGEVFGGGTKLTVL |
| | (SEQ ID NO: 93) |
| N1wc145 | HVILTQPRSVSGSPGQSITISCTGTSSDVGGYNYVSWYQQHPGKAPKLMIYEGSK |
| | RPSGVSNRFSGSKSGNTASLTISGLQAEDEADYYCSSYTTRSTRVFGGGTKLTVL |
| | (SEQ ID NO: 94) |
| N1wc152 | QAVLTQPSSVSGAPGQRVTISCTGSSSNIGAPFDVHWYQQLPGTAPKLLIYGNSN |
| | RPSGVPDRFSGSKFGTSASLAITGLQAADEADYYCQSYDSSLSGWVFGGGTKLTVL |
| | (SEQ ID NO: 95) |
| N1wc157 | SYELTQPPSVSVSPGQTARITCSGDALPDKYVHWYQQKSGQAPVLVLFDDSKRPS |
| | GIPERFSGSSSGTVATLIISGAQVEDEGDFFCYSTDSSGNERVFGGGTKLTVL |
| | (SEQ ID NO: 96) |
| N1wc158 | QSVLTQPPSASGTPGQRVTISCSGSSSNIGSNSVYWYQQLPGTAPKLLIYRNNQR |
| | PSGVPDRFSGSKSGTSASLAISGLRSEDEADYYCAAWDDSLSGLVFGGGTKLTVL |
| | (SEQ ID NO: 97) |
| N1wc160 | SYVLTQPPSASGTPGQRVTISCSGSSSNIGSNTVHWYQQLPETAPKLLIYTNNQRP |
| | SGVPDRFSGSKSGTSASLAISGLQSEDEADYYCASWDDSLKAYVFGTRTKLTVL |
| | (SEQ ID NO: 98) |
| N1wc175 | QSVLTQPPSASGTPGQRVTISCSGSRSSIGSNTVSWYQQLPGTAPKLIIYTNNQRP |
| | SGVPDRFSGSKSGTSATLAISGLQSEDEADYYCASWDDSLNDYVFGVGTKVTVL |
| | (SEQ ID NO: 99) |
| N1wc179 | SYVLTQPPSASGTPGQRVTISCSGSSSNIGSNTVNWYQQLPGTAPKLLIYNDNERP |
| | SGVPDRFSGSKSGTYASLAISGLQSEDEADYYCAAWDDSLNGLAFGGGTKLTVL |
| | (SEQ ID NO: 100) |
| N1wc191 | LPVLTQPPSASGTPGQRVTISCSGSSSNIGSNIVNWYQQLPGTAPKLLIYTSNQRP |
| | SGVPDRFSGSKSGTSASLAISGLQSEDEADYYCAAWDDSLNAVVFGGGTKVTVL |
| | (SEQ ID NO: 101) |
| N1wc202 | SYELTQPPSVSGTPGQRVTISCSGSSSNIESNAVHWYQHLPGRAPKLLIFSHNQRP |
| | SGVPARFSGSKSGTSASLAISGLQSEDEADYYCAAWDDSLSGYVFASGTKLTVL |
| | (SEQ ID NO: 102) |
| N1wc520 | SYVLTQPPSVSGTPGQRVTISCSGSSSNIESNAVHWYQHLPGRAPKLLIFSHNQRP |
| | SGVPARFSGSKSGTSASLAISGLQSEDEADYYCAAWDDSLSGYVFASGTKVTVL |
| | (SEQ ID NO: 103) |
| N1wc625 | QSVLTQPPSASGTPGQRVTISCSGGRSNIGTNYVYWYQKLPGTAPKSLIYTTNQR |
| | PSGVPDRFSGSKAGTSASLAISGLRSEDEGDYYCASWDESLNGVVFGGGTKVTVL |
| | (SEQ ID NO: 104) |

B. DNA sequence
B.1- Binding to LBD (N1-EGF 1-13)

Heavy Chain
>hN1S3-009
GAAGTGCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGTTGAGCAGGCTGAGATC
TGACGACACGGCCGTGTATTACTGTGCGACAGACTCCTTTGACTACTGGGGCCAGGGGACAATGGT
CACCGTCTCGAGT (SEQ ID NO: 105)
>hN1S3-010
GAGGTGCAGCTGGTGCAGTCTGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCATCATGACCAGGGACACGTCCATCAGCACAGCCTACATGGAACTGAGCAGGCTGACATC
TGACGACACGGCCGTGTATTACTGTGCGACAGACTCCTTTGACTACTGGGGCAGAGGGACAATGGT
CACCGTCTCGAGT (SEQ ID NO: 106)
> hN1S3-011
CAGGTGCAGCTGGTGCAGTCTGGGCCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCGGGGGACAATGGT
CACCGTCTCGAGT (SEQ ID NO: 107)
> hN1S3-050
CAGGTGCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCGGGGGACAATGGT
CACCGTCTCGAGT (SEQ ID NO: 108)
> hN1S3-052
CAGGTCCAGCTGGTACAGTCTGGAGCTGAGGTGAAGAAGCCTGGGGCCTCAGTGAGGATTTCCTGC
AAGACATCTGGATTCACCTTTACCAGCTACTTTATCCACTGGGTGCGGCAGGCCCCAGGACAAAGG
CCAGAGTGGATGGGCGTTATCAACCCAAGCCGTGGCAACGCAGACTACGCACCGAGGTTCCGTGG
CAGAGTCACCATGACCCGGGACAAGTCCACGCACACTGTTTACATGGATCTGAAGAGCCTCAGATCT
GACGACGCGGCCATATATTACTGTGCCAGAGATCGGCTTGGTGGCCTTCTTGATTACTACGACAGTA
GTGATTCTTCGAAGAATTTTGATGCTTTTGATTTCTGGGGCCAAGGCACCCTGGTCACCGTCTCGAGT
(SEQ ID NO: 109)
> hN1S3-094
GAGGTGCAGCTGGTGCAGTCTGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCGGGGGACAATGGT
CACCGTCTCGAGT (SEQ ID NO: 110)
> hN1S3-113
GAGGTGCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCGGGGCACCCTGGT
CACCGTCTCGAGT (SEQ ID NO: 111)
>hN1S3-126
GAGGTGCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCAGGGCACCCTGGT
CACCGTCTCGAGT (SEQ ID NO: 112)
> hN1S3-146
GAGGTGCAGCTGGTGCAGTCTGGGCCTGAGGTGAAGAAGCCTGGGACCTCAGTGAAGGTCTCCTG
CAAGGCTTCTGGATTCACCTTTACTAGCTCTGCTGTGCAGTGGGTGCGACAGGCTCGTGGACAACG
CCTTGAGTGGATAGGATGGATCGTCGTTGGCAGTGGTAACACAAACTACGCACAGAAGTTCCAGGA
AAGAGTCACCATTACCAGGGACATGTCCACAAGCACAGCCTACATGGAGCTGAGCAGCCTGAGATC
CGAGGACACGGCCGTGTATTATTGTGTGGCAGATACGGTCTACTGGGGCAAGGGAACCCTGGTCAC
CGTCTCGAGT (SEQ ID NO: 113)
> hN1S3-174
GAAGTGCAGCTGGTGCAGTCTGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
ACTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGGCAAGGGACCACGGT
CACCGTCTCGAGT (SEQ ID NO: 114)
> hN1S3-056
CAGGTACAGCTGCAGCAGTCAGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
ACTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCGGGGAACCCTGGT
CACCGTCTCGAGT (SEQ ID NO: 115)
> hN1S3-013
GAGGTGCAGCTGGTGCAGTCTGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCGAGGAACCCTGGT
CACCGTCTCGAGT (SEQ ID NO: 116)
>N1wc613
CAGGTCCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCGGGGCACCCTGGT
CACCGTCTCGAGT (SEQ ID NO: 117)
>N1wc133
CAGGTGCAGCTGGTGCAATCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCAGGGCACCCTGGT
CACCGTCTCGAGT (SEQ ID NO: 118)
>N1wc173
GAGGTGCAGCTGGTGCAGTCTGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGGCAGGGGACCACGGT
CACCGTCTCGAGT (SEQ ID NO: 119)
>N1wc155
GAGGTGCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCGGGGAACCCTGGT
CACCGTCTCGAGT (SEQ ID NO: 120)
>N1wc168
GAGGTCCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCGGGGCACCCTGGT
CACCGTCTCGAGT (SEQ ID NO: 121)
>N1wc159
CAGCTGCAGCTGGTGGAGACCGGGGGAGGCCTGGTCAAGCCTGGGGAGTCCCTGACTCTCTCCTG
TGCAGCCTCTGGATTCACCTTCAGTAGTTATTCCATGAACTGGGTCCGCCAGGCTCCAGGGAAGGG
GCTGGAGTGGGTCTCATCCATTAGTAGTAGTGGTAGTTCCATATACTACGCAGACTCATTGAAGGGC
CGATTCACCATCTCCAGAGACAACGCCAAGAACTCACTGTATCTGCAAATGAACAGCCTGAGAGCCG
AGGACACGGCTGTCTATTACTGTACGAGAGGGACGCAGTGGCTGGCTGGGGTAGGAGACTATTGG
GGCAAGGGAACCCTGGTCACCGTCTCGAGT (SEQ ID NO: 122)
>N1wc97
GAGGTCCAGCTGGTGCAGTCTGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCGCCATGACCAGGGACACGTCCATCAGCACAGCCTACATGGAGCTGAGCAGGCTGAGAT
CTGACGACTCGGCCGTGTATTACTGTGCGACAGACTCCTTTGACTACTGGGGGCGAGGGACCACGG
TCACCGTCTCGAGT (SEQ ID NO: 123)
>N1wc49
GAGGTGCAGCTGGTGGAGTCTGGGGCTGAGGTGAAGAAGCCTGGGGCCTCACTTAACCTTTCCTG
CAAGGCATCTGGATACTCCTTCAACAAATACTACATCCACTGGGTGCGACAGGCCCCTGGACAAGG
ACTTGAGTTGATGGGACGGATCAACCCTGGAAGTGGTACCACAAGCTACGCACAGAAATTCCAGGG
CAGAGTCACCCTGTCCAGGGACACGTCGACGAACACCATCTTCATGGAACTGAACGGCCTCACAGG
TGAAGACACGGCCATATATTATTGTGCGAGGTCGAACTACGATATATTGGCTGGTTATGGGCCTGAC
GCTTTTGATATCTGGGGCCGAGGCACCCTGGTCACCGTCTCGAGT (SEQ ID NO: 124)
Light Chain
> hN1S3-009
TCCTATGTGCTGACTCAGCCACCCTCGGTGTCTGAAGCCCCCAGGCAGAGGGTCACCATCTCCTGT
TCTGGAAGCAGCTCCAACATCGGAAATAATGCTGTAAGCTGGTACCAGCAGCTCCCAGGAAAGGCT
CCCAAAACCCTCATCTATTATGATGATCTGCTGTCCTCAGGGGTCTCTGACCGATTCTCTGGCTCCA
GGTCTGGCACCTCAGCCTCCCTGGCCATCAGTGGGCTCCAGTCCGAGGATGAGGCTGATTATTACT
GTGCAGCATGGGATGACAGCCTGAATGGTGTGGTATTCGGCGGAGGGACCAAGCTGACCGTCCTA
(SEQ ID NO: 125)
> hN1S3-010
CAGTCTGTGCTGACGCAGCCGCCCTCAGCGTCTGGGACCCCCGGGCAGAGGGTCACCATCTCTTG
TTCTGGAAGCAACTCCAACATCGGCAGTAATTATGTATTCTGGTACCAGCAGCTCCCGGGAACGGCC
CCCAAACTCCTCATCTATAACAATAATCAGCGGCCCTCAGGGGTCCCTGACCGATTCTCTGGCTCCA
AGTCTGGCACCTCAGCCTCCCTGGCCATCAGTGGGCTCCGGTCCGAGGATGAGGCTGATTATTACT
GTGCAGCATGGGATGACAGCCTGAGTGGTGTGGTATTCGGCGGAGGGACCAAGCTGACCGTCCTA
(SEQ ID NO: 126)
> hN1S3-011
CAGTCTGTGCTGACTCAGCCACCCTCAGCGTCTGGGACCCCCGGGCAGAGGGTCACCATCTCTTGT
TCTGGAAGCAGCTCCAACATCGGAAGTAATACTGTAAACTGGTACCAGCAGCTCCCAGGAACGGCC
CCCAAACTCCTCATCTATAGTAATAATCAGCGGCCCTCAGGGGTCCCTGACCGATTCTCTGGCTCCA
AGTCTGGCACCTCAGCCTCCCTGGCCATCAGTGGGCTCCAGTCTGAGGATGAGGCTGATTATTACT
GTGCAGCATGGGATGACAGCCTGAATGGTGTGGTATTCGGCGGAGGGACCAAGCTGACCGTCCTA
(SEQ ID NO: 127)
> hN1S3-050
CTGCCTGTGCTGACTCAGCCACCCTCAGCGTCTGGGACCCCCGGGCAGAGGGTCACCATCTCTTGT
TCTGGAAGGAGCTCCAACATCGGAAGTAATTTTGTATATTGGTACCAGCAGCTCCCAGGAACGGCCC
CCAAACTCCTCATCTACAGGAATAATCAGCGGCCCTCAGGGGTCCCTGACCGATTCTCCGGCTCCA
AGTCTGGCACCTCAGCCTCCCTGGCCATCAGTGGGCTCCGGTCCGAGGATGAGGCTGATTATTATT
GTGCAACATGGGATGACAACCTGAGTGGTGTGGTATTCGGCGGAGGGACCAAGCTGACCGTCCTA
(SEQ ID NO: 128)
> hN1S3-052
CAGGCTGTGCTGACTCAGCCGTCCTTAGCGTCTGGGACCCCCGGGCAGAGGGTCACCATCTCTTGT
TCTGGAGGCAGTTCCAACATCGGAAGACATACTGTGAACTGGTACCAGCAACTCCCAGGAGCGGCC
CCCAAACTCCTCATGCATAGTAGTGATCAGCGGCCCTCAGGGGTCCCTGACCGATTCTCTGGCTCC
AAGTCTGGGACCTCAGCCTCCCTGGCCATCAGTGGGCTCCAGTCTGAGGATGAGGCTGATTATTAC
TGTGCAGCATGGGATGCCAGTCTCAAGAATTGGGTGTTCGGCGGAGGGACCAAGCTGACCGTCCTA
(SEQ ID NO: 129)
> hN1S3-094
CAGTCTGTGCTGACGCAGCCGCCCTCAGCGTCTGGGACCCCCGGGCAGAGGGTCACCATCTCTTG
TTCTGGAGGCAGTTCCAACATCGGAAGTAATACTGTAACCTGGTACCAACAACTCCCAGGAAAGGCC
CCCAAACTCCTCATTTATATCAGTGATCAGCGGCCCTCTGGGGTCCCTGACCGATTCTCTGGCTCCA
AGTCTGGCACCTCAGCCTCCCTGGCCATCAGAGGGCTCCAGTCTGAGGATGAGGCTGATTATTACT
GTGCAGCATGGGATGACAGCCTCAATGGTGCCGTCTTCGGAACTGGGACCAAGGTCACCGTCCTA
(SEQ ID NO: 130)
> hN1S3-113
TCCTATGTGCTGACTCAGCCACCCTCAGCGTCTGGGACCCCCGGGCAGAGGGTCACCATCTCTTGT
TCTGGGAGCAGTCCCAACATCGGAAGTAATACTGTCAACTGGTACCAGCAACTCCCAGGAACGGCC
CCCAAACTTCTCATCTATACTAATAATCAGCGGCCCTCAGGGGTCCCTGACCGATTCTCTGGCTCCA
AGTCTGCTACCTCAGCCTCCCTGGCCATCAGTGGGCTCCAGTTTGACGATGAGGCTGATTATTTCTG
TGCAGCGTGGGACGACAGCCTTCTTGGTGTGGTCTTCGGCGGAGGGACCAAGGTCACCGTCCTA
(SEQ ID NO: 131)
> hN1S3-126
TCCTATGTGCTGACTCAGCCACCCTCAGCGTCTGGGGCCCCCGGTCAGAGGGTCACCATCTCTTGT
TCTGGTAGCATCTCCAACATCGGAGATAATACTGTAAACTGGTACCAACACCTCCCAAGAACGGCCC
CCAAACTCGTCATCTATAGTGATGTTCAGCGGCCCTCCGGGGTCCCTGAGCGATTCTCTGGCTCCAA
GTCTGGCACCTCAGCCTCCCTGGTCATCAGTGGACTCCAGGCTGAAGATGAGGCTGATTATTACTGT
GCATCATGGGACGACAGCCTGGATGGTGTGTTTTTCGGCGGAGGGACCAAGCTGACCGTCCTA
(SEQ ID NO: 132)
> hN1S3-146
CAGGCTGTGCTGACTCAGCCGTCCTCGGTGTCTGAGGCCCCCAGGCAGAGGGTCACCATCTCCTGT
TCTGGAAGCAGCTCCAACATCGGAAAGAATGCTGTAAACTGGTACCAGCAGCTCCCAGGGAAGGCT
CCCAAACTCCTCATCTATTATGATGATCTGATGCCCTCAGGGGTCTCTGACCGATTCTCTGGCTCCA
AGTCTGGCACCTCAGCCTCCCTGGCCATCAGTGGGCTCCAGTCTGAGGATGAGGCTGACTATTTCT
GTGCAGCATGGGATGACAGCCTGAGTGGTTGGGTGTTCGGCGGAGGGACCAAGCTGACCGTCCTA
(SEQ ID NO: 133)
> hN1S3-174
CAGTCTGTGTTGACGCAGCCGCCCTCAGTGTCTGGGGCCCCAGGGCAGAGGATCACCATCTCCTG
CACTGGGAGCAGCTCCAACATCGGGGCAGGTTATGATGTACACTGGTACCAGCAGCTTCCAGGAAC
AGCCCCCAAACTCCTCATCTATGGTAACAGCTATCGGCCCTCAGGGGTCCCTGACCGATTCTCTGG
CTCCAAGTCTGGCACCTCAGCCTCCCTGGCCATCACTGGGCTCCAGGCTGAGGATGAGGCTGATTA
TTACTGCCAGTCCTATGACAGCAGCCTGACTGGTTCGATATTCGGCGGAGGGACCAAGCTGACCGT
CCTA (SEQ ID NO: 134)
> hN1S3-056
CAGTCTGTGCTGACTCAGCCACCCTCAGCGTCTGGGACCCCCGGGCAGAGGGTCACCATCTCTTGT
TCTGGAAGCAGCTCCAACATCGGAAGTAATACTGTAAACTGGTACCAGCAGCTCCCAGGAACGGCC
CCCAAACTCCTCATCTATAGTAATAATCAGCGGCCCTCAGGGGTCCCTGACCGATTCTCTGGCTCCA
AGTCTGGCACCTCAGCCTCCCTGGCCATCAGTGGGCTCCAGTCTGAGGATGAGGCTGATTATTACT
GTGCAGCATGGGATGACAGCCTGAATGGGTTGGTATTCGGCGGAGGGACCAAGGTCACCGTCCTA
(SEQ ID NO: 135)
> hN1S3-013
CAGCCTGTGCTGACTCAGCCACCCTCAGCGTCTGGGACCCCCGGGCAGAGGGTCACCATCTCTTGT
TCTGGAAGCAGCTCCAACATCGGAAGTAATTATGTATATTGGTACCAGAAGTTCCCAGGAACGGCCC
CCAAACTCCTCATCTATACGAATAATCAGCGGCCCTCAGGGGTCCCTGACCGATTTTCTGGCTCCAA
GTCTGGCACCTCAGCCTCCCTGGCCATCAGTGGGCTCCGGTCCGAGGATGAGGCTGATTATTACTG
TGCAGCATGGGATGACAGCCTGAGTGGTGTGGTATTCGGCGGAGGGACCAAGGTCACCGTCCTA
(SEQ ID NO: 136)
>N1wc613
TCCTATGAGCTGACTCAGCCACCCTCAGCGTCTGGGACCCCCGGGCAGAGGGTCACCATCTCTTGT
TCTGGAAGCTACTCCAACATCGGAAGTAATTATGTATACTGGTACCAGCAGCTCCCAGGAACGGCCC
CCAAACTCCTCATCTATACGAATAATCAGCGGCCCTCAGGGGTCCCTGACCGATTCTCTGGCTCCAA
GTCTGGCACCTCAGCCTCCCTTGTCATCAGTGGGCTCCGGTCCGACGATGAAGGTGATTATTACTGT
GCAGCATGGGATGACAGTGTGAGTGTCCTGTTATTCGGCGGGGGGACCAAGGTCACCGTCCTA
(SEQ ID NO: 137)
>N1wc133
TCCTATGAGCTGACTCAGCCACCCTCAGCGTCTGGGACCCCCGGGCAGAGGGTCACCATCTCTTGT
TCTGGAAGCAACTCCAACATCGGAAGTAATACTCTAAACTGGTACCAACAACTCCCAGGAACGGCCC
CCAAACTCCTCATCTATAGTAATAATCAGCGGCCCTCATGGGTCCCTGACCGATTCTCTGGCTCCAA
GTCTGGCACCTCAGCCTCCCTGGCCATCAGTGGTCTCCAGTCTGAGGATGAGGCTGATTATTACTGT
GCAGCATGGGATGACAGCCTGAATGGCCTGATATTCGGCGGAGGGACCAAGCTGACCGTCCTA
(SEQ ID NO: 138)
>N1wc173
CAGCCTGTGCTGACTCAGCCCCCCTCAGCGTCTTGGACCCCCGGGCAGAGGGTCACCATCTCTTGT
TCTGGAAGCAGTTCCAACATCGGAAGTAATACAGTGCACTGGTACCGGCAGTTCCCAGGAACGGCC
CCCCAACTCCTCATCTATAGTAATAACCAGCGGCCCTCAGGGGTCCCTGACCGATTCTCTGGCTCCA
AGTCTGGCACCTCAGCCTCCCTGGCCATCAGAGGGCTCCAGTCTGAGGATGAGGCTGATTATTATT
GTGCAGCATGGGATGACAGCCTGAATGGTGTGGTTTTCGGCGGAGGGATCAAGCTGACCGTCCTA
(SEQ ID NO: 139)
>N1wc155
CAGTCTGTGTTGACGCAGCCGCCCTCAGTGTCTGGGGCCCCAGGGCAGAGGGTCACCATCTCCTG
CACTGGGAGCAGCTCCAACATCGGGGCAGGTTATGATGTACTCTGGTACCGGCAGCTTCCAGGAAC
AGCCCCCAAACTCCTCATCTATGGTAACACCCATCGGCCCTCAGGGGTCCCTGACCGATTCTCTGG
CTCCAACTCTGGCACCTCAGCCTCCCTGGCCATCACTGGGCTCCAGGCTGAGGATGAGGCTGATTA
TTACTGCCAGTCCTATGACAGCAGCCTGAGTGGTTCGGTGTTCGGCGGAGGGACCAAGCTGACCGT
CCTA (SEQ ID NO: 140)
>N1wc168
CAGTCTGTGTTGACGCAGCCGCCCTCAGCGTCTGGGACCCCCGGGCAGAGGGTCACCATCTCTTGT
TCTGGAAGCAGCTCCAACATCGGAAGTAATATTGTAAACTGGTACCAGCAGCTCCCAGGAACGGCC
CCCAAACTCCTCATCTATAGTAATAATCAGCGGCCCTCAGGGGTCCCTGACCGATTCTCTGGCTCCA
AGTCTGGCACCTCAGCCTCCCTGGCCATCAGTGGGCTCCAGTCTGAGGATGAGGCTGATTATTACT
GTGCAGCATGGGATGACAGCCTGAATGGTGTGGTATTCGGCGGAGGGACCAAGCTGACCGTCCTA
(SEQ ID NO: 141)
>N1wc159
CAATCTGCCCTGACTCAGCCTCCCTCCGCGTCCGCGTCTCCTGGCCAGTCAGTCACCATCTCCTGC
ACTGGAACCAGCAGTGACGTTGGTGCTTATGACTCTGTCTCCTGGTACCAACAGCACCCCGGCAAA
GCCCCCAAACTCATAATTTATGACGTCAATAACCGGCCCTCAGGGGTTTCGAATCGCTTCTCTGGCT
CCGAGTCTGGCAACACGGCCTCCCTGACCATCTCTGGGCTCCAGGCTGAGGACGAGGCTAATTATT
ACTGCAGCTCATATAGAAACACCAAGACTGTGGTATTCGGCGGGGGGACCAAGCTGACCGTCCTA
(SEQ ID NO: 142)
>N1wc97
CAGTCTGTGCTGACTCAGCCACCCTCGGTGTCTGGGGCCCCAGGCCAGAGGGTCACCATCTCCTG
CACTGGGACCAGCTCCAACATCGGGGCAGGTTATGATGTACAGTGGTACCAGCAGCTTCCAGGAAC
AGCCCCCAAAGTCCTCATGTATGGTAACAGCAATCGGCCCTCAGGGGTCCCTGACCGATTCTCAGG
CTCCAAGTCTGGCACCTCAGCCTCCCTGGCCATCACTGGGCTCCAGGCTGAGGATGAGGCTGATTA
TTATTGCCAGTCCTATGACAGCAGCCTGAGTGGTTCCCTCTTCGGAACCGGGACCAAGGTCACCGT
CCTA (SEQ ID NO: 143)
>N1wc49
CAGTCTGTCGTGACGCAGCCGCCCTCGGTGTCTGGGGCTCCGGGGCAGACAGTCACCATCTCCTG
CACTGGGAATAGCTCCAACATCGGGTCAAGTGTTGATGTACACTGGTACCAGCAGCTTCCAGGGAC
AGCCCCCAAACTCCTCATCTATGATAACAAAAATCGGCCCTCGGGGGTCCCTGACCGATTCTCTGGC
TCCAAGTCTGGCACCTCAGCCTCCCTGGCCATCACTGGGCTCCAGGCTGAGGATGAGGCTGATTAT
TACTGCCAGTCCTATGACAGCAGCCTGAGTTCTGAGGTGTTCGGCGGAGGGACCAAGGTCACCGTC
CTA (SEQ ID NO: 144)

B.2- Binding to NRR

Heavy Chain
>N1wc629
GAGGTCCAGCTGGTACAGTCTGGGGCTGAGGTGAAGAAGCCTGGGTCCTCGGTGAAGGTCTCCTG
CAAGGCTTCTGGAGGCACCCTCAGCAGCTACACTGTCAGCTGGTTGCGACAGGCCCCTGGACAAG
GTCTTGAGTGGATGGGACGGATTATCCCTATCCTTGATAGAGCAAACTATGCACAGAAGTTCCAGGG
CAGAGTCACGATTACCGCGGACAAATCCACGAGCACAGCCTACATGGAGCTCAACAGCCTGAGATC
GGACGACACGGCCGTATATTACTGTGCGAGAAGTATAGGAGCAGCTGGCGATGGTGTTTGGTTCGA
CCCCTGGGGCCAGGGGACAATGGTCACCGTCTCGAGT (SEQ ID NO: 145)
>N1wc75
GGGGTGCAGCTGGTGCAGTCTGGGGGAGGCTTGGTACAGCCTGGGGGGTCCCTGAGACTCTCCTG
TGCAGCCTCTGGATTCACCTTTGATGATTATGCCATGCACTGGGTCCGGCAAGCTCCAGGGAAGGG
CCTGGAGTGGGTCTCAAGTATTAGTTGGCATAGTCGTACCATAGCCTATGCGGACTCTGTGAAGGG
CCGATTCTCCATCTCCAGAGACAACGCCAAGAACTCCCTCTATCTGCAAATGAACAGTCTGAGACCT
GAGGACACGGCCGTATATTACTGTGCGAAAGCCTCGTACCTCAGCACCTCGTCCTCCCTTGACTATT
GGGGCCGAGGAACCCTGGTCACCGTCTCGAGT (SEQ ID NO: 146)
>N1wc104
CAGGTGCAGCTGCAGGAGTCCGGCCCAGGACTGGTGAAGCCTTCGGGGACCCTGTCCCTCACCTG
CGCTGTCTCTGGTGTCTCCCTCGCCACTAATAACTGGTGGCTTTGGGTCCGCCAGTCCCCAGGGAA
GGGACTGGAGTGGATTGGAGAAATTTATCATAGTGGATACACCAACTACAACCCGTCCCTCAAGAGT
CGAGTCACCATATCCGTAGACAAGTCCAGCAACCAGCTCTCCCTGGACCTGAGGTCTATCACCGCC
GCGGACACGGCCGTTTATTATTGTGCGAGAAGATATTGTGATAATGGTGTTTGTTATCCCTTTGACCA
CTGGGGCCAGGGGACAATGGTCACCGTCTCGAGT (SEQ ID NO: 147)
Light Chain
>N1wc629
CAGGCTGTGCTGACTCAGCCGTCCTCAGTGTCTGGGGCCCCAGGGCAGAGGGTCACCATCTCCTG
CACTGGGAGCAGCTCCAACATCGGGGCAGGTTATGATGTACACTGGTACCAGCAGCTTCCAGGAAC
AGCCCCCAAACTCCTCATCTTTGATAACAAGAATCGGCCCTCAGGGGTCCCTGACCGATTCTCTGGC
TCCAACTCTGGCACCTCAGCCTCCCTGGCCATCACTGGGCTCCAGGCTGAAGATGAGGCTGAATAT
TACTGCCAGTCGTATGACAACAACCTGAGTGGCCGGGTGTTCGGCGGAGGGACCAAGCTGACCGT
CCTA (SEQ ID NO: 148)
>N1wc75
CAGTCTGTGCTGACTCAGCCTGGCTCCGTGTCTGGGTCTCCTGGACAGTCGATCACCATCTCCTGC
ACTGGAACCAGCAGTGACGTTGGTGGTTATAACTATGTCTCCTGGTACCAACAACACCCAGGCAAAG
CCCCCAAACTCATGATTTATGAGGGCAGTAAGCGGCCCTCAGGGGTTTCTAATCGCTTCTCTGGCTC
CAAGTCTGGCAACACGGCCTCCCTGACAATCTCTGGGCTCCAGGCTGAGGACGAGGCTGATTATTA
CTGCAGCTCATATACAACCAGGAGCACTCGAGTTTTCGGCGGAGGGACCAAGCTGACCGTCCTA
(SEQ ID NO: 149)
>N1wc104
TCCTATGTGCTGACTCAGCCACCCTCGGTGTCTGAAGCCCCCAGGCAGACGGTCACCATCTCCTGT
TCTGGAAACAGTTTCAACATCGGGAGATATCCTGTCAACTGGTATCAACAACTCCCAGGGAAGGCTC
CCAAACTCCTCATTTATTATAATAATCTGAGGTTTTCAGGGGTCTCTGACCGATTCTCTGGCTCCAAG
TCTGGCACCTCAGCCTCCCTGGCCATCAGGGATCTCCTGTCTGAGGACGAGGCTGATTACTATTGTT
CGACGTGGGATGACACCCTGAAGGGTTGGGTGTTCGGCGGAGGGACCAAGCTGACCGTCCTA
(SEQ ID NO: 150)

B.3- Additional Notch Antibodies

Heavy Chain
> hN1S3-067
GAGGTGCAGCTGGTGCAGTCTGGGGCCGAGGTGAAGAAGCCTGGGGCCTCAGTGAGGATTTCCTG
CAAGACATCTGGATTCACCTTTACCAGCTACTTTATCCACTGGGTGCGGCAGGCCCCAGGACAAAG
GCCAGAGTGGATGGGCGTTATCAACCCAAGCCGTGGCAACGCAGACTACGCACCGAGGTTCCGTG
GCAGAGTCACCATGACCCGGGACAAGTCCACGCACACTGTTTACATGGATCTGAAGAGCCTCAGAT
CTGACGACGCGGCCATATATTACTGTGCCAGAGATCGGCTTGGTGGCCTTCTTGATTACTACGACAG
TAGTGATTCTTCGAAGAATTTTGATGCTTTTGATTTCTGGGGCCAAGGAACCCTGGTCACCGTCTCGA
GT (SEQ ID NO: 151)
> hN1S3-073
CAGGTCCAGCTGGTGCAGTCTGGGGCTGAGGTGAAGAAGCCTGGGGCCTCAGTGAGGATTTCCTG
CAAGACATCTGGATTCACCTTTACCAGCTACTTTATCCACTGGGTGCGGCAGGCCCCAGGACAAAG
GCCAGAGTGGATGGGCGTTATCAACCCAAGCCGTGGCAACGCAGACTACGCACCGAGGTTCCGTG
GCAGAGTCACCATGACCCGGGACAAGTCCACGCACACTGTTTACATGGATCTGAAGAGCCTCAGAT
CTGACGACGCGGCCATATATTACTGTGCCAGAGATCGGCTTGGTGGCCTTCTTGATTACTACGACAG
TAGTGATTCTTCGAAGAATTTTGATGCTTTTGATTTCTGGGGCCAGGGAACCCTGGTCACCGTCTCG
AGT (SEQ. ID. NO. 152)
> hN1S3-116
GAAGTGCAGCTGGTGCAGTCTGGGGCTGAGGTGAAGAAGCCTGGGGCCTCAGTGAAGGTCTCCTG
CAAGGCTTCTGGATACACCTTCACCGATTACTATATGCACTGGGTACGACAGGCCCCTGGACAAGGA
CTTGAGTGGATGGGATGGATCAACCCTAACAGTGGTGGCACATACTTTCCACAGAACTTTCAGGGCC
GGGTCACCATGACCAGCGACCCGTCCATCAGCGCAGCCTACATGGAATTGAGTAACCTGAGATCTG
ACGACACGGCCGTGTATTTCTGTGCAAGAGGCGCTTACGATTACGTTTGGGGGAGTCATCGTTATAG
CGGCGATGCTTTCGATTTCTGGGGCCAAGGAACCCTGGTCACCGTCTCCTCA (SEQ ID NO: 153)
> hN1S3-193
CAGGTGCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCAGGGAACCCTGGT
CACCGTCTCGAGT (SEQ ID NO: 154)
>N1wc14
GAAGTGCAGCTGGTGCAGTCTGGGGGAGGCTTGGTACAGCCCGGCAGGTCCCTGAGACTCTCCTG
TGCGGCCTCTGGATTCACCTCTGATGATTATGCCATGCACTGGGTCCGGCAAGCTCCAGGGAAGGG
CCTGGACTAGGTCACAGCTATCACTTGGAATAGTGGTCACAAGGACTATGCGGACTCTGTGAAGGG
CCGATTCGCCGTCTCCAGAGACAACGCCAAGAACGCCCTGTATCTGCAAATGAACAGTCTGAGACC
TGAGGACACGGCCGTATATTACTGTGCGAAAGCCTCGTACCTCAGCACCTCGTCCTCCCTTGACTAC
TGGGGCAGAGGGACAATGGTCACCGTCTCGAGT (SEQ ID NO: 155)
>N1wc23
GAAGTCCAGCTGGTGCAGTCTGGGGCTGAGGTGAAGAAGCCTGGGGCCTCAGTGAAGGTCTCCTG
CAAGGCTTCTGGATACAACTTCACCGGCTACTATATACACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCATCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCAGAGGAACCCTGGT
CACCGTCTCGAGT (SEQ ID NO: 156)
>N1wc33
GAGGTGCAGCTGGTGGAGTCTGGGGGAGACTTAGTTCAGCCTGGGGGGTCCCTGAGACTCTCCTG
TGCAGCCTCTGGATTCACCTTCAGTGATTCCTGGATGCACTGGGTCCGCCAAGTTCCAGGGAAGGG
ACTGGAGTGGGTCTCACATATTGGTATTGATGGGATTACCACAAACTACGCGGACTCCGTGAAGGG
CCGATTCACCATCTCCAGGGACAACGCCAAGAACACCCTGTATCTGCAAATGAACAATCTGAGAGCC
GAAGACACGGCTGTATATTACTGTGCCAGGAATGTAGGGTGGGAGTCTCGCCCTTGGGGCCAAGGA
ACCCTGGTCACCGTCTCGAGT (SEQ ID NO: 157)
>N1wc48
CAGGTCCAGCTGGTGCAGTCTGGAGCTGAGGTGAAGAAGCCTGGGGCTACAGTGAAAATCTCCTGC
AAGGTTTCTGGATACACCTTCACCGACTACTACATCCACTGGGTGCAACAGGCCCCTGGAAAAGGG
CTTGAGTGGATGGGACTTGTTGATCCTGAAGATGGTGAAACAATATACGCAGAGAAGTTTCAGGGCA
GAGTCACCATAATTGCGGACACGTCTACAGACACAGCCTACATGGAACTGAGCAGCCTGAGATCTG
AGGACACGGCCGTCTATTACTGTGCAACTGGGCCGTTCCGTCTTGACTCCTGGGGCCAAGGGACAA
TGGTCACCGTCTCGAGT (SEQ ID NO: 158)
>N1wc60
CAGGTGCAGCTGGTGCAGTCTGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
ACTTGAGTGGATGGGATGGATCAACCCTAACAGTGGTGGCACAAACTATGCACAGAAGTTTCAGGG
CAGGGTCACCATGACCAGGGAAACGTCCATCAGCACAGCCTACATGGAGCTGAGCAGGCTGAGATC
TGACGACACGGCCGTGTATTACTGTGCGACAGACTCCTTTGACTACTGGGGCCGAGGCACCCTGGT
CACCATCTCGAGT (SEQ ID NO: 159)
>N1wc99
CAGGTCCAGCTGGTGCAGTCTGGGGCTGAGGTGAAGAAGCCTGGGGCCTCAGTGAAGGTCTCCTG
CAAGGCTTCTGGTTACACCTTTACCAGCTATGGTATCAGCTGGGTGCGACAGGCCCCTGGACAAGG
GCTTGAGTGGATGGGATGGATCAGCGCTTACAATGGTAACACAAACTATGCACAGAAGCTCCAGGG
CAGAGTCACCATGACCACAGACACATCCACGAGCACAGCCTACATGGAGCTGAGGAGCCTGAGATC
TGACGACACGGCCGTGTATTACTGTGCGAGACTGGCCAGCCGTGCTTTTGATATCTGGGGCAGGGG
CACCCTGGTCACCGTCTCGAGT (SEQ ID NO: 160)
>N1wc103
CAGGTACAGCTGCAGCAGTCAGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCTATCAGCACAGCCTACGTGGAGCTGAGCAGGCTGAGATC
TGACGACACGGCCGTGTATTACTGTGCGACAGACTCCTTTGACTACTGGGGCCGGGGCACCCTGGT
CACCGTCTCGAGT (SEQ ID NO: 161)
>N1wc106
CAGGTCCAGCTGGTACAGTCTGGGGCTGAGGTGAAGAGGCCTGGGGCCTCAGTTAACCTTTCCTGC
AAGGCATCTGGATACTCCTTCAGCAGATACTATATCCACTGGGTGCGACAGGCCCCTGGACAGGGA
CTTGAGTTGATGGGACGGATCAACCCTGGAGGTGGTACCACAACCTACGCACAGAAATTCCAGGGC
AGAGTCACCCTGTCCAGGGACACGTCCACGAACACCATCTTCATGGAACTGAACGGCCTCACATCT
GAAGACACGGCCATGTATTATTGTGCGAGGTCGAACTACGATATTTTGGCTGGTTATGGGCCTGATG
CTTTTGATTTCTGGGGCCGGGGCACCCTGGTCACCGTCTCGAGT (SEQ ID NO: 162)
>N1wc110
GAAGTGCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCAGGGGACAATGGT
CACCGTCTCGAGT (SEQ ID NO: 163)
>N1wc112
GAGGTGCAGCTGGTGGAGTCTGGGGGGGGCTTGGCTCAGCCTGGAAAGTCCCTCAGAATATCCTG
TGCAGCCTCTGGATTCACCTTTGGCGACTACGCCATGACGTGGGTCCGCCAGGCTCCAGGGAAGG
GACTGGAGTGGGTCTCAAGTATAAGTGGTAGTGGTTCTGGCACATATTATACAGACTCCGTGAAGGG
CCGCTTCGCCATCTCCAGAGACAACTCCAAGAACACACTATATCCGCAGATGAACAGCCTGGCAGC
CGAGGACACGGCCATTTATTTCTGTGCAAAAGATCAGACAGATGTGACTATCCGGGACTTTGACTAC
TGGGGCAGGGGAACCCTGGTCACCGTCTCGAGT (SEQ ID NO: 164)
>N1wc137
CAGGTGCAGCTGGTGCAGTCTGGAGCTGAGGTGAAGAAGCCTGGGGCCTCAGTGAAGGTCTCCTG
CAAGGCTTCTGGTTACACCTTTACCAACTATGGTCTCAGCTGGGTGCGACAGGCCCCTGGACAAGG
GCTTGAGTGGATGGGATGGATCAGCGGTTACAATGATAACACAAGCTATGCACAGAAGTTCCAGGA
CAGAATCACTATGACCACAGACACATCCACGAGCACAGCCTCCATGGAGCTGAGGAGCCTGAGATC
TGACGACACGGCCGTGTATTACTGTGCGTTTAGTTATTATGATAGTAGTGGTTATTTCAACTGGGGCC
AGGGGACAATGGTCACCGTCTCGAGT (SEQ ID NO: 165)
>N1wc140
CAGGTACAGCTGCAGCAGTCAGGGGCTGAGGTAAAGAAGCCTGGGTCCTCGGTGAAGGTCTCCTG
CAGGGCTTCTGGAGGCACCTTCAGCAACTACGCTATCAGCTGGGTGCGACAGGCCCCTGGACAAG
GCCTTGAGTGGATGGGACGGATCATCCCTATCTTTGGAGGAGCACACTACGCACAGAAGTTACAGG
GCAGAGTCTCGATTACCGCGGACGAGTCGACGAGCACAGCCCACATGGAGCTGAGCAGCCTGAGA
TCTGAGGACGCGGCCGTGTACTACTGTGCGAGCGGAGCGTATTACGATGTTATGGGTAATTATCCTT
ACTCAGGTATGGACGTCTGGGGCCGAGGCACCCTGGTCACCGTCTCGAGT (SEQ ID NO: 166)
>N1wc142
GAGGTGCAGCTGGTGCAGTCTGGGGCTGAGGTGAAGAAGCCTGGGGCCTCAGTTAACCTTTCCTG
CAAGGCATCTGGATACTCCTTCAGCAGGTACTATATCCACTGGGTGCGACAGGCCCCTGGACAAGG
ACTTGAGTTGATGGGACGGATCAACCCTGGAGGTGGTACCACAACCTACGCACAGAAATTCCAGGG
CAGAGTCACCCTGTCCAGGGACACGTCCACGAACACCATCTTCATGGAACTGAACGGCCTCACATC
TGAAGACACGGCCATGTATTATTGTGCGAGGTCGAACTACGATATTTTGGCTGGTTATGGGCCTGAT
GCTTTTGATTTCTGGGGCCGAGGGACAATGGTCACCGTCTCGAGT (SEQ ID NO: 167)
>N1wc145
GAGGTGCAGCTGGTGGAGTCTGGAGCTGAGGTGAAGAAGCCTGGGGCCTCAGTGAAGGTCTCCTG
CAAGGCTTCTGGTTACACCTTTACCACCTATGGTATCAGCTGGGTGCGACAGGCCCCTGGACAAGG
GCTTGAGTGGATGGGATGGATCAGCACTTACAATGGTAACACAAAATATGCACAGAAGCTCCAGGG
CAGAGTCACCATGACCACAGACACATCCACGAGCACAGCCTACATGGAGCTGAGGAGCCTGAGATC
TGACGACACGGCCGTGTATTACTGTGCGAGAGATGCAAGGAAAGCCTTTGATATCTGGGGCAGAGG
CACCCTGGTCACCGTCTCTTCA (SEQ ID NO: 168)
>N1wc152
CAGGTGCAGCTGGTGCAGTCTGGGGGAGGGTTGGTCCAGCCTGGGGGGTCCCTAAGACTGTCCTG
TGGAGCCTCTGGGTTCACCTTTAGTAGCCATTGGATGAGCTGGGTCCGCCGGCCTCCAGGGAGGG
GGCTGGAATGGGTGGCCAACATAAACGAGGGTGGCAGTGCGGAATATTATGCGGACTCTCTGAAGG
GCCGATTCACCATCTCCCGAGACAACGCCAAGAACTCCCTGTTTCTGCAAATGGACAGCCTGAGAG
CCGAGGACACGGCCGTCTATTACTGTGTGAGAGATCAATTCCACAGTAACTACGACTGGGGCCAGG
GGACAATGGTCACCGTCTCGAGT (SEQ ID NO: 169)
>N1wc157
CAGGTCCAGCTGGTGCAGTCTGGAGCTGGGCTGAAGAGGCCGGGGACCTCAGTGAAAATATCCTG
CAAGACTTCTGGATACATCTTCAGTCAATATCCCATGCACTGGGTGCGCCAGGCCCCCGGACAAGG
GCTTGAGTGGGTGGCATGGGTCGACACTGGCAATGGTACCACAAGATATTCGCCGAATTTCCAGGG
CAGAGCCACCGTTAGTGGAGACACATCCGCGAACACAGGCTACTTGGAATTGAGAAGCCTGAGATT
TACCGACACGGCTGTTTATTACTGTGCGACTAACGCCTTTGACTACTGGGGCCGAGGCACCCTGGT
CACCGTCTCGAGT (SEQ ID NO: 170)
>N1wc158
GAGGTCCAGCTGGTACAGTCTGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCGAGGCACCCTGGT
CACCGTCTCGAGT (SEQ ID NO: 171)
>N1wc160
CAGGTGCAGCTGCAGGAGTCCGGAGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCGGGGGACAATGGT
CACCGTCTCGAGT (SEQ ID NO: 172)
>N1wc175
CAGGTCCAGCTGGTGCAGTCTGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCGGGGCACCCTGGT
CACCGTCTCGAGT (SEQ ID NO: 173)
>N1wc179
GAGGTCCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCAGGGGACAATGGT
CACCGTCTCGAGT (SEQ ID NO: 174)
>N1wc191
CAGGTGCAGCTGGTGCAATCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCAAGGGACAATGGT
CACCGTCTCGAGT (SEQ ID NO: 175)
>N1wc202
GAGGTGCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCGGGGCACCCTGGT
CACCGTCTCGAGT (SEQ ID NO: 176)
>N1wc520
CAGGTGCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGGCGAGGGACCACGGT
CACCGTCTCGAGT (SEQ ID NO: 177)
>N1wc625
CAGGTGCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCAGGGGACAATGGT
CACCGTCTCGAGT (SEQ ID NO: 178)
Light Chain
> hN1S3-067
AATTTTATGCTGACTCAGCCCCACTCTGTGTCGGAGTCTCCGGGGAAGACGGTAATCATCTCCTGCA
CTCGCAGCAGTGGCAGCATTGCCAGCAACTATGTGCAGTGGCTCCAGCAGCGCCCGGGCAGTTCC
CCCACCACTGTGATTTATGACGATAATCGAAGACCCTCTGGAGTCCCTGATCGGTTTTCTGGCTCCA
TCGACGGTTCCTCCAACTCGGCCTCCCTCACCATCTCTGGCCTGAAGACAGAGGACGAGGCTGATT
ACTACTGTCAGTCTTATGATCGATTCGACCATGTGGTTTTCGGCGGAGGGACCAAGCTGACCGTCCTA
(SEQ ID NO: 179)
> hN1S3-073
GACATCCAGATGACCCAGTCTCCATCCTCACTGTCTGCATTTGTGGGAGACAGAGTCACCATCACTT
GTCGGGCGAGTCAGGGCATTAATAATTATTTAGCCTGGTTTCAGCAGAAGCCAGGGAAAGCCCCTAA
GTCCCTGATCTACGCCGCCTCCACTTTGCGAAGTGGGGTCCCACCAAGGTTCAGCGGCAGTGGATC
TGGGACAGATTTCACTCTCACCATCAGCAGCCTGCAGCCTGAAGATTTTGCAACTTATTACTGCCAA
CAGTATGATAGTTACCCTCACACCTTCGGCCAGGGGACACGACTGGAGATTAAA (SEQ ID NO: 180)
> hN1S3-116
CAGTCTGTGCTGACTCAGCCACCCTCAGCGTCTGGGACCCCCGGGCAGAGGGTCACCATCTCTTGT
TCTGGAAGCAGCTCCAACATCGGGAGTAACACTGTAAATTGGTACCAGCGACTCCCAGGAGCGGCC
CCCCAACTCCTCATCTACAATAATGACCAGCGGCCCTCAGGGATCCCTGACCGATTCTCTGGCTCCA
AGTCTGGCACCTCAGGTTCCCTGGTCATCAGTGGGCTCCAGTCTGAAGATGAGGCTGATTACTACTG
TGCGTCATGGGATGACAGTCTGAATGGTCGGGTGTTCGGCGGAGGGACCAAGCTGACCGTCCTA
(SEQ ID NO: 181)
> hN1S3-193
TCCTATGAGCTGACTCAGCCACCCTCAGCGTCTGGGACCCCCGGGCAGAGGGTCACCATCTCTTGT
TCTGGAAGCAGCTCCAACATCGGAAGTAATACTGTAAACTGGTACCAGCAGCTCCCAGGAACGGCC
CCCAAACTCCTCATCTATAGTAATAATCAGCGGCCCTCAGGGGTCCCTGACCGATTCTCTGGCTCCA
AGTCTGGCACCTCAGCCTCCCTGGCCATCAGTGGGCTCCAGTCTGAGGATGAGGCTGATTATTACT
GTGCAGCATGGGATGACAGCCTGAATGGTTATGTCTTCGGAACTGGGACCAAGCTGACCGTCCTA
(SEQ ID NO: 182)
>N1wc14
TCCTATGTGCTGACTCAGCCACCCTCGGTGTCAGTGGCCCCAGGACAGACGGCCAGGATTACCTGT
GGGGGAAACAACATTGGAAGTAAAAGTGTGCACTGGTACCAGCAGAAGCCAGGCCAGGCCCCTGT
GCTGGTCATCTATGAGGACAGCAAACGACCCTCCGGGATCCCTGAGAGATTCTCTGGCTCCAGCTC
AGGGACAATGGCCACCTTGACTATCAGTGGGGCCCAGGTGGAGGATGAAGCTGACTACTACTGTTA
CTCAACAGACAGCAGTGGTAATCATAGGGTCTTCGGAACTGGGACCAAGGTCACCGTCCTA
(SEQ ID NO: 183)
>N1wc23
CAGTCTGTGCTGACTCAGCCACCCTCAGCGTCTGGGACCCCCGGGCAGAGGGTCACCATCTCTTGT
TCTGGAAGCTACTCCAACATCGGAAGTAATTATGTCTACTGGTACCAGCAGTTCCCAGGAACGGCCC
CCAAACTCCTCATCTATACGAATAATCAGCGGCCCTCAGGGGTCCCTGACCGATTCTCTGGCTCCAA
GTCTGGCTCCTCAGCCTCCCTTGCCATCAGTGGGCTCCGGTCCGAGGATGAGGCTGATTATTACTG
TGCAGCATGGGATGACAGTGTGGGTGTCCTGTTATTCGGCGGGGGGACCAAGCTGACCGTCCTA
(SEQ ID NO: 184)
>N1wc33
TCCTATGTGCTGACTCAGCCACCCTCAGCGTCTGGGGCCCCCGGCCAGAGGGTCACCATCTCTTGT
TCTGGAAGCGACTCCAACATCGGACGTAACACTGTTAACTGGTACCAGCAGCAGCTCCCTGGAACG
GCCCCCACACTCCTCATCTATACTAATAATCAGCGGCCCTCAGGGGTCCCTGACCGATTCTCTGGCT
CCAAGTCTGGCACCTCAGCCTCCCTGGCCATCAGTGGGCTCCAGTCTGGGGATGAGGCTGATTATT
ACTGTGCAGCATGGGATGACAGCCTCAATGCTTATTTTTTCGGGCCTGGGACCAAGCTGACCGTCCTA
(SEQ ID NO: 185)
>N1wc48
CAGTCTGTGTTGACGCAGCCGCCCTCAGTTTCTGGGGCCCCAGGGCAGAGGGTCACCATCTCCTGC
ACTGGGAGCAGCTCCAACATCGGGGCAGGTTATGATGTACACTGGTACCAGCAGCTTCCAGGATCA
GCCCCCAAACTCCTCATCTATGGTAACAACAATCGGCCCTCAGGGGTCCCTGACCGATTCTCTGGCT
CCAAGTCTGGCACCTCAGCCTCCCTGGCCATCACTGGGCTCCAGGCTGAGGATGAGGCTGATTATT
ACTGCCAGTCCTTTGACAACAGGCTGAGTGATTTTAATGTCTTCGGAACTGGGACCAAGGTCACCGT
CCTA (SEQ ID NO: 186)
>N1wc60
CAGTCTGTGCTGACGCAGCCGCCCTCAGCGTCTGGGACCCCCGGGCAGAGGGTCACCATCTCTTG
TTCTGGAAGCAGCTCCAACATCGGAAGTAATTATGTATACTGGTACCAGCAGCTCCCAGGAACGGCC
CCCAAACTCCTCATCTATAGGAATAATCAGCGGCCCTCAGGGGTCCCTGACCGATTCTCTGGCTCCA
AGTCTGGCACCTCAGCCTCCCTGGCCATCAGTGGGCTCCGGTCCGAGGATGAGGCTGATTATTACT
GTGCAGCATGGGATGACAGCCTGAGTGGTCTGGTATTCGGCGGAGGGACCAAGCTGACCGTCCTA
(SEQ ID NO: 187)
>N1wc99
CAGTCTGTGCTGACTCAGCCTGCCTCCGTGTCTGGGTCTCCTGGACAGTCGATCACCATCTCCTGC
ACTGGAACCAGCAGTGACGTTGGTGGTTATAACTATGTCTCCTGGTACCAACAACACCCAGGCAAAG
CCCCCAAACTCATGATTTATGAGGGCAGTAAGCGGCCCTCAGGGGTTTCTAATCGCTTCTCTGGCTC
CAAGTCTGGCAACACGGCCTCCCTGACAATCTCTGGGCTCCAGGCTGAGGACGAGGCTGATTATTA
CTGCAGCTCATATACAACCAGGAGCACTCGAGTTTTCGGCGGAGGGACCAAGCTGACCGTCCTA
(SEQ ID NO: 188)
>N1wc103
CAGTCTGTGTTGACGCAGCCGCCCTCAGCGTCTGGGACCCCCGGGCAGAGGGTCACCATCTCTTGT
TCTGGAAGCAGCTCCAACATCGGAAGTAATCCTGTAAACTGGTACCAGCAGCTCCCAGGAACGGCC
CCCAAACTCCTCATCTATAGTAATAATCAGCGGCCCTCAGGGGTCCCTGACCGATTCTCTGGCTCCA
AGTCTGGCACCTCAGCCTCCCTGGCCATCAGTGGGCTCCACTCTGAGGATGAGGCTGATTATTACT
GTGCAGCATGGGATGACAGCCTGAATGGGTCTTCTGTCTTCGGAACTGGGACCAAGCTGACCGTCC
TA (SEQ ID NO: 189)
>N1wc106
CAGGCTGTGCTGACTCAGCCGTCCTCAGTGTCTGGGGCCCCAGGGCAGAGGGTCACCATCTCCTG
CACTGGGAGCAGCTCCAACATCGGGGCAGATAATGATGTTCATTGGTACCAAAAGTTTCCAGGAACA
GCCCCCAAACTCCTCATCTTTTCTAACAACAATCGGCCCTCAGGGGTCCCTGACCGGTTCTCTGGCT
CCAAGTCTGGCACCTCAGCCTCTCTGGCCATCACTGGACTCCAGGCTGACGATGAGGCTGATTATTA
CTGCCAGTCATATGATAGCGGCCTGACGAGGGAGTTGTTCGGCGGAGGGACCAAGCTGACCGTCC
TA (SEQ ID NO: 190)
>N1wc110
CAGTCTGTCGTGACGCAGCCGCCCTCAGCGTCTGGGTCCCCCGGGCAGAGGGTCACCATCGCTTG
TTCTGGAAGCAGCTCCAACATCGGAAGTAATACTGTAAACTGGTACCAGCACGTTCCAGGAACGGCC
CCCAAACTCCTCATCTATAATAATAATCAGCGGCCCTCAGGGGTCCAAGATCGATTCTCTGGCTCCA
AGTCTGACACTTCAGCCTCCCTGGCCATCAGTGGTCTCCAGGCTGAGGATGAGGCTGTTTATTACTG
TGCGACATGGGATGACAGCCTGATTGGTCTAGCGTTCGGCGGAGGGACCAAGCTGACCGTCCTA
(SEQ ID NO: 191)
>N1wc112
TCCTATGAGCTGACTCAGCCACCCTCGGTGTCGGTGGCCCCGGGACAGACGGCCAGGATTTCCTGT
GGGGGAGACAACCTTGGACGTAAAAGCGTGCACTGGTACCAGCAGAAGCCAGGCCAGGCCCCTGT
GCTGGTCATCTATGAGGACAGCAAACGACCCTCCGGGATCCCTGAGAGATTCTCTGGCTCCAGCTC
AGGGACAATGGCCACCTTGACTATCAGTGGGGCCCAGGTGGAGGATGAAGCTGACTACTACTGTTA
CTCAACAGACAGCAGTGGTAATCATAGGGTGTTCGGCGGAGGGACCAAGGTCACCGTCCTA
(SEQ ID NO: 192)
>N1wc137
CAGGCTGTGCTGACTCAGCCGTCCTCAGTGTCTGGGGCCCCAGGGCAGAGGGTCACCGTCTCCTG
CACTGGGAGCAGCTCCAATATCGGGACAGGTTATGCTGTACACTGGTACCAGCAGCTTCCAGGAAC
AGCCCCCAGACTCCTCATCTTTGGAAACAACAATCGGCCCTCAGGGGTCCCTGACCGATTCTCTGG
CTCCAAGTCTGGCACCTCAGCCTCCCTGGTCATCACTGGGCTCCAGGCTGAGGATGAGGCTGATTA
TTACTGCCAGTCCTTTGACACCCTTAGTGGTTCGAGGGTGTTCGGCGGAGGGACCAAGGTCACCGT
CCTA (SEQ ID NO: 193)
>N1wc140
TCCTATGTGCTGACTCAGCCACCCTCGGTGTCAGTGTCCCCCGGCCAGACGGCCAAGATCACCTGT
GGGGGAGACAAGATTGAGACAAAAAGTGTGCACTGGTACCAGCAGAAGCCGGGCCAGGCCCCTGT
TCTGGTCATCTATGAGGACACCAAACGACCCTCCGGGATCTCTGAGAGATTGTCGGGCTCCAGCGC
AGGGACAGTGGCCACGTTGACCATCACTGGGGCCCAGGTGGACGATGAGGCTGACTACTTTTGTTA
TTCCACAGACGCCACCGGTACTGAAAGGGTCTTCGGCGGAGGGACCAAGCTGACCGTCCTA
(SEQ ID NO: 194)
>N1wc142
CAGTCTGTGCTGACTCAGCCACCCTCAGTGTCTGGGGCCCCAGGGCAGAGGGTCACCATCTCCTGC
ATTGGGAGCAACTCCAACATCGGGGCAGGTCATGATGTACACTGGTACCAGCAGTTTCCAGGAACA
GCCCCCAAACTCCTCATCTATGATAACACCAACCGGCCCTCAGGGGTCCCTGACCGATTCTCTGGCT
CCAAGTCTGGCACCTCAGTTTCCCTGGCCATCACTGGGCTCCAGGCTGAGGATGAGGCTGATTATT
ACTGCCAGTCCTATGACAGCAGCCTGGGTGAGGTGTTCGGCGGAGGGACCAAGCTGACCGTCCTA
(SEQ ID NO: 195)
>N1wc145
CACGTTATACTGACTCAACCTCGCTCAGTGTCTGGGTCTCCTGGACAGTCGATCACCATCTCCTGCA
CTGGAACCAGCAGTGACGTTGGTGGTTATAACTATGTCTCCTGGTACCAACAACACCCAGGCAAAGC
CCCCAAACTCATGATTTATGAGGGCAGTAAGCGGCCCTCAGGGGTTTCTAATCGCTTCTCTGGCTCC
AAGTCTGGCAACACGGCCTCCCTGACAATCTCTGGGCTCCAGGCTGAGGACGAGGCTGATTATTAC
TGCAGCTCATATACAACCAGGAGCACTCGAGTTTTCGGCGGAGGGACCAAGCTGACCGTCCTA
(SEQ ID NO: 196)
>N1wc152
CAGGCTGTGCTGACTCAGCCGTCCTCAGTGTCTGGGGCCCCAGGGCAGAGGGTCACCATCTCCTG
CACTGGGAGCAGCTCCAACATCGGGGCACCTTTTGATGTACACTGGTACCAGCAGCTTCCAGGAAC
AGCCCCCAAACTCCTCATCTATGGTAACAGTAATCGGCCCTCAGGAGTCCCTGACCGCTTCTCTGGC
TCCAAGTTTGGCACCTCAGCCTCCCTGGCCATCACTGGGCTCCAGGCTGCGGATGAGGCTGATTAT
TACTGCCAGTCTTATGACAGCAGCCTGAGTGGTTGGGTGTTCGGCGGAGGGACCAAGCTGACCGTC
CTA (SEQ ID NO: 197)
>N1wc157
TCCTATGAGCTGACTCAGCCACCCTCGGTGTCAGTGTCCCCAGGACAAACGGCCAGGATCACCTGC
TCTGGAGATGCATTGCCAGACAAATATGTTCATTGGTACCAGCAGAAGTCAGGCCAGGCCCCTGTCC
TGGTCCTCTTCGACGACAGTAAGCGACCCTCCGGTATCCCTGAGAGATTCTCTGGCTCCAGCTCAG
GGACAGTGGCCACCTTGATTATCAGTGGGGCCCAGGTGGAGGATGAAGGTGACTTCTTCTGTTACT
CAACAGACAGCAGTGGTAATGAAAGGGTTTTCGGCGGAGGGACCAAGCTGACCGTCCTA
(SEQ ID NO: 198)
>N1wc158
CAGTCTGTGCTGACTCAGCCACCCTCAGCGTCTGGGACCCCCGGGCAGAGGGTCACCATCTCTTGT
TCTGGAAGCAGCTCCAACATCGGAAGTAATTCTGTATACTGGTACCAGCAGCTCCCAGGAACGGCC
CCCAAACTCCTCATCTATAGGAATAATCAGCGGCCCTCAGGGGTCCCTGACCGATTCTCTGGCTCCA
AGTCTGGCACCTCAGCCTCCCTGGCCATCAGTGGGCTCCGGTCCGAGGATGAGGCTGATTATTACT
GTGCAGCATGGGATGACAGCCTGAGTGGTCTGGTCTTCGGCGGAGGGACCAAGCTGACCGTCCTA
(SEQ ID NO: 199)
>N1wc160
TCCTATGTGCTGACTCAGCCACCCTCAGCGTCTGGGACCCCCGGGCAGAGGGTCACCATCTCTTGT
TCTGGGAGCAGCTCCAACATCGGAAGTAATACTGTACACTGGTACCAGCAGCTCCCAGAAACGGCC
CCCAAACTCCTCATCTATACTAATAATCAGCGGCCCTCAGGGGTCCCTGACCGATTCTCTGGCTCCA
AGTCTGGCACCTCAGCCTCCCTGGCCATCAGTGGGCTCCAGTCTGAGGACGAGGCTGACTATTACT
GTGCTTCATGGGATGACAGCCTGAAAGCTTATGTCTTCGGAACTAGGACCAAGCTGACCGTCCTA
(SEQ ID NO: 200)
>N1wc175
CAGTCTGTGCTGACTCAGCCACCCTCTGCGTCTGGGACCCCCGGGCAGAGGGTCACCATCTCTTGT
TCTGGAAGCAGGTCCAGCATCGGAAGTAATACTGTAAGCTGGTACCAGCAACTCCCAGGAACGGCC
CCCAAACTCATCATCTATACTAATAATCAGCGGCCCTCCGGGGTCCCTGACCGATTCTCTGGCTCCA
AGTCTGGCACCTCAGCCACCCTGGCCATCAGTGGGCTCCAGTCTGAGGATGAGGCTGATTATTACT
GTGCATCATGGGATGACAGCCTGAATGATTATGTCTTCGGAGTTGGGACCAAGGTCACCGTCCTA
(SEQ ID NO: 201)
>N1wc179
TCCTATGTGCTGACTCAGCCACCCTCAGCGTCTGGGACCCCCGGGCAGAGGGTCACCATCTCTTGT
TCTGGAAGCAGCTCCAACATCGGAAGTAATACTGTAAACTGGTACCAGCAGCTCCCAGGAACGGCC
CCCAAACTCCTCATCTATAATGATAATGAGCGGCCCTCTGGGGTCCCTGACCGATTCTCGGGCTCCA
AGTCTGGCACCTACGCCTCCCTGGCCATCAGTGGGCTCCAGTCTGAGGATGAGGCTGATTATTACT
GTGCAGCTTGGGATGACAGCCTGAATGGTCTGGCTTTCGGCGGAGGGACCAAGCTGACCGTCCTA
(SEQ ID NO: 202)
>N1wc191
CTGCCTGTGCTGACTCAGCCCCCCTCAGCGTCTGGGACCCCCGGGCAGAGGGTCACCATCTCTTGT
TCTGGAAGCAGCTCCAACATCGGAAGTAATATTGTAAACTGGTACCAGCAGCTCCCAGGAACGGCC
CCCAAACTCCTCATCTATACTAGTAATCAGCGGCCCTCAGGGGTCCCTGACCGATTCTCTGGCTCCA
AGTCTGGCACCTCAGCCTCCCTGGCCATCAGTGGGCTCCAGTCTGAGGATGAGGCTGATTATTACT
GTGCAGCATGGGATGACAGCCTGAATGCTGTGGTATTCGGCGGAGGGACCAAGGTCACCGTCCTA
(SEQ ID NO: 203)
>N1wc202
TCCTATGAGCTGACTCAGCCACCCTCAGTTTCTGGGACCCCCGGGCAGAGGGTCACCATCTCTTGT
TCTGGAAGCAGTTCCAACATCGAAAGTAATGCTGTCCATTGGTACCAACACCTCCCAGGAAGGGCCC
CCAAACTCCTCATCTTTAGTCATAATCAGCGGCCCTCAGGTGTCCCTGCCCGCTTCTCTGGCTCCAA
GTCTGGCACCTCAGCCTCCCTGGCCATCAGTGGGCTCCAGTCTGAGGATGAGGCTGACTATTACTG
TGCAGCTTGGGATGACAGCCTGAGTGGTTATGTCTTCGCATCTGGGACCAAGCTGACCGTCCTA
(SEQ ID NO: 204)
>N1wc520
TCCTATGTGCTGACTCAGCCACCCTCAGTTTCTGGGACCCCCGGGCAGAGGGTCACCATCTCTTGTT
CTGGAAGCAGTTCCAACATCGAAAGTAATGCTGTCCATTGGTACCAACACCTCCCAGGAAGGGCCC
CCAAACTCCTCATCTTTAGTCATAATCAGCGGCCCTCAGGTGTCCCTGCCCGCTTCTCTGGCTCCAA
GTCTGGCACCTCAGCCTCCCTGGCCATCAGTGGGCTCCAGTCTGAGGATGAGGCTGACTATTACTG
TGCAGCTTGGGATGACAGCCTGAGTGGTTATGTCTTCGCATCTGGGACCAAGGTCACCGTCCTA
(SEQ ID NO: 205)
>N1wc625
CAGTCTGTGCTGACTCAGCCACCCTCAGCGTCTGGGACCCCCGGGCAGAGGGTCACCATCTCTTGT
TCTGGAGGCAGATCCAATATCGGAACTAATTATGTGTACTGGTACCAGAAATTACCAGGCACGGCCC
CCAAATCCCTCATCTATACTACTAATCAGCGCCCCTCAGGGGTCCCTGACCGATTCTCTGGCTCCAA
GGCTGGCACCTCAGCCTCCCTGGCCATCAGTGGGCTCCGGTCCGAGGATGAGGGTGATTATTACTG
TGCCTCGTGGGATGAAAGCCTGAATGGTGTGGTGTTCGGCGGAGGGACCAAGGTCACCGTCCTA
(SEQ ID NO: 206)

C. CDRs (Aminoacid sequences)
C.1- Binding to LBD (N1-EGF 1-13)

Heavy Chain

| Clone Name | HC-CDR1 | HC-CDR2 | HC-CDR3 |
|---|---|---|---|
| hN1S3-009 | GYNFTGYY (SEQ ID | INPNSGG (SEQ | ATDSFDY (SEQ ID NO: 209) |
| | NO: 207) | ID NO: 208) | |
| hN1S3-010 | GYNFTGYY (SEQ ID | INPNSGG (SEQ | ATDSFDY (SEQ ID NO: 209) |
| | NO: 207) | ID NO: 208) | |
| hN1S3-011 | GYNFTGYY (SEQ ID | INPNSGG (SEQ | ATDSFDY (SEQ ID NO: 209) |
| | NO: 207) | ID NO: 208) | |
| hN1S3-013 | GYNFTGYY (SEQ ID | INPNSGG (SEQ | ATDSFDY (SEQ ID NO: 209) |
| | NO: 207) | ID NO: 208) | |
| hN1S3-050 | GYNFTGYY (SEQ ID | INPNSGG (SEQ | ATDSFDY (SEQ ID NO: 209) |
| | NO: 207) | ID NO: 208) | |
| hN1S3-052 | GFTFTSYF (SEQ ID | INPSRGN (SEQ | ARDRLGGLLDYYDSSDSSKNFDAFDF |
| | NO: 210) | ID NO: 211) | (SEQ ID NO: 212) |
| hN1S3-056 | GYNFTGYY (SEQ ID | INPNSGG (SEQ | ATDSFDY (SEQ ID NO: 209) |
| | NO: 207) | ID NO: 208) | |
| hN1S3-094 | GYNFTGYY (SEQ ID | INPNSGG (SEQ | ATDSFDY (SEQ ID NO: 209) |
| | NO: 207) | ID NO: 208) | |
| hN1S3-113 | GYNFTGYY (SEQ ID | INPNSGG (SEQ | ATDSFDY (SEQ ID NO: 209) |
| | NO: 207) | ID NO: 208) | |
| hN1S3-126 | GYNFTGYY (SEQ ID | INPNSGG (SEQ | ATDSFDY (SEQ ID NO: 209) |
| | NO: 207) | ID NO: 208) | |
| hN1S3-146 | GFTFTSSA (SEQ ID | IVVGSGN (SEQ | VADTVY (SEQ ID NO: 215) |
| | NO: 213) | ID NO: 214) | |
| hN1S3-174 | GYNFTGYY (SEQ ID | INPNSGG (SEQ | ATDSFDY (SEQ ID NO: 209) |
| | NO: 207) | ID NO: 208) | |
| N1wc97 | GYNFTGYY (SEQ ID | INPNSGG (SEQ | ATDSFDY (SEQ ID NO: 209) |
| | NO: 207) | ID NO: 208) | |
| N1wc613 | GYNFTGYY (SEQ ID | INPNSGG (SEQ | ATDSFDY (SEQ ID NO: 209) |
| | NO: 207) | ID NO: 208) | |
| N1wc133 | GYNFTGYY (SEQ ID | INPNSGG (SEQ | ATDSFDY (SEQ ID NO: 209) |
| | NO: 207) | ID NO: 208) | |
| N1wc155 | GYNFTGYY (SEQ ID | INPNSGG (SEQ | ATDSFDY (SEQ ID NO: 209) |
| | NO: 207) | ID NO: 208) | |
| N1wc159 | GFTFSSYS (SEQ ID | ISSSGSS (SEQ | TRGTQWLAGVGDY |
| | NO: 216) | ID NO: 217) | (SEQ ID NO: 218) |
| N1wc168 | GYNFTGYY (SEQ ID | INPNSGG (SEQ | ATDSFDY (SEQ ID NO: 209) |
| | NO: 207) | ID NO: 208) | |
| N1wc173 | GYNFTGYY (SEQ ID | INPNSGG (SEQ | ATDSFDY (SEQ ID NO: 209) |
| | NO: 207) | ID NO: 208) | |
| N1wc49 | GYSFNKYY (SEQ ID | INPGSGT (SEQ | ARSNYDILAGYGPDAFDI (SEQ ID |
| | NO: 219) | ID NO: 220) | NO: 221) |

Light Chain

| Clone Name | LC-CDR1 | LC-CDR2 | LC-CDR3 |
|---|---|---|---|
| hN1S3-009 | SSNIGNNA (SEQ | YD | AAWDDSLNGVV (SEQ |
| | ID NO: 222) | | ID NO: 223) |
| hN1S3-010 | NSNIGSNY (SEQ | NN | AAWDDSLSGVV (SEQ |
| | ID NO: 224) | | ID NO: 225) |
| hN1S3-011 | SSNIGSNT (SEQ | SN | AAWDDSLNGVV (SEQ |
| | ID NO: 226) | | ID NO: 227) |
| hN1S3-013 | SSNIGSNY (SEQ | TN | AAWDDSLSGVV (SEQ |
| | ID NO: 228) | | ID NO: 225) |
| hN1S3-050 | SSNIGSNF (SEQ | RN | ATWDDNLSGVV (SEQ |
| | ID NO: 229) | | ID NO: 230) |
| hN1S3-052 | SSNIGRHT (SEQ | SS | AAWDASLKNWV (SEQ |
| | ID NO: 231) | | ID NO: 232) |
| hN1S3-056 | SSNIGSNT (SEQ | SN | AAWDDSLNGLV (SEQ ID |
| | ID NO: 226) | | NO: 233) |
| hN1S3-094 | SSNIGSNT (SEQ | IS | AAWDDSLNGAV (SEQ |
| | ID NO: 226) | | ID NO: 234) |
| hN1S3-113 | SPNIGSNT (SEQ | TN | AAWDDSLLGVV (SEQ ID |
| | ID NO: 235) | | NO: 236) |
| hN1S3-126 | ISNIGDNT (SEQ | SD | ASWDDSLDGVF (SEQ |
| | ID NO: 237) | | ID NO: 238) |
| hN1S3-146 | SSNIGKNA (SEQ | YD | AAWDDSLSGWV (SEQ |
| | ID NO: 239) | | ID NO: 240) |
| hN1S3-174 | SSNIGAGYD | GN | QSYDSSLTGSI (SEQ ID |
| | (SEQ ID NO: 241) | | NO: 242) |
| N1wc97 | SSNIGAGYD | GN | QSYDSSLSGSL (SEQ ID |
| | (SEQ ID NO: 241) | | NO: 243) |
| N1wc613 | YSNIGSNY (SEQ | TN | AAWDDSVSVLL (SEQ ID |
| | ID NO: 244) | | NO: 245) |
| N1wc133 | NSNIGSNT (SEQ | SN | AAWDDSLNGLI (SEQ ID |
| | ID NO: 246) | | NO: 247) |
| N1wc155 | SSNIGAGYD | GN | QSYDSSLSGSV (SEQ ID |
| | (SEQ ID NO: 241) | | NO: 248) |
| N1wc159 | SSDVGAYDS | DV | SSYRNTKTVV (SEQ ID |
| | (SEQ ID NO: 249) | | NO: 250) |
| N1wc168 | SSNIGSNI (SEQ | SN | AAWDDSLNGVV (SEQ |
| | ID NO: 251) | | ID NO: 227) |
| N1wc173 | SSNIGSNT (SEQ | SN | AAWDDSLNGVV (SEQ |
| | ID NO: 226) | | ID NO: 227) |
| N1wc49 | SSNIGSSVD | DN | QSYDSSLSSEV (SEQ ID |
| | (SEQ ID NO: 252) | | NO: 253) |

C.2- Binding to NRR
Heavy Chain

| Clone Name | HC-CDR1 | HC-CDR2 | HC-CDR3 |
|---|---|---|---|
| N1wc104 | GVSLATNNW | IYHSGY (SEQ ID | ARRYCDNGVCYPFDH (SEQ ID |
| | (SEQ ID NO: 254) | NO: 255) | NO: 256) |
| N1wc629 | GGTLSSYT (SEQ | IIPILDR (SEQ ID | ARSIGAAGDGVWFDP (SEQ ID |
| | ID NO: 257) | NO: 258) | NO: 259) |
| N1wc75 | GFTFDDYA (SEQ | ISWHSRT (SEQ | AKASYLSTSSSLDY (SEQ ID |
| | ID NO: 260) | ID NO: 261) | NO: 262) |

Light Chain

| Clone Name | LC-CDR1 | LC-CDR2 | LC-CDR3 |
|---|---|---|---|
| N1wc104 | SFNIGRYP (SEQ ID | YN | STWDDTLKGWV |
| | NO: 263) | | (SEQ ID NO: 264) |
| N1wc629 | SSNIGAGYD (SEQ ID | DN | QSYDNNLSGRV |
| | NO: 241) | | (SEQ ID NO: 265) |
| N1wc75 | SSDVGGYNY (SEQ | EG | SSYTTRSTRV (SEQ |
| | ID NO: 266) | | ID NO: 267) |

C.3- Additional Notch Antibodies
Heavy Chain

| Clone Name | HC-CDR1 | HC-CDR2 | HC-CDR3 |
|---|---|---|---|
| hN1S3-067 | GFTFTSYF | INPSRGN | ARDRLGGLLDYYDSSDSSKNFDAFDF |
| | (SEQ ID | (SEQ ID | (SEQ ID NO: 212) |
| | NO: 210) | NO: 211) | |
| hN1S3-116 | GYTFTDYY | INPNSGG | ARGAYDYVWGSHRYSGDAFDF (SEQ |
| | (SEQ ID | (SEQ ID | ID NO: 269) |
| | NO: 268) | NO: 208) | |
| hN1S3-073 | GFTFTSYF | INPSRGN | ARDRLGGLLDYYDSSDSSKNFDAFDF |
| | (SEQ ID | (SEQ ID | (SEQ ID NO: 212) |
| | NO: 210) | NO: 211) | |
| hN1S3-193 | GYNFTGYY | INPNSGG | ATDSFDY (SEQ ID NO: 209) |
| | (SEQ ID | (SEQ ID | |
| | NO: 207) | NO: 208) | |
| N1wc520 | GYNFTGYY | INPNSGG | ATDSFDY (SEQ ID NO: 209) |
| | (SEQ ID | (SEQ ID | |
| | NO: 207) | NO: 208) | |
| N1wc625 | GYNFTGYY | INPNSGG | ATDSFDY (SEQ ID NO: 209) |
| | (SEQ ID | (SEQ ID | |
| | NO: 207) | NO: 208) | |
| N1wc103 | GYNFTGYY | INPNSGG | ATDSFDY (SEQ ID NO: 209) |
| | (SEQ ID | (SEQ ID | |
| | NO: 207) | NO: 208) | |
| N1wc106 | GYSFSRYY | INPGGGT | ARSNYDILAGYGPDAFDF (SEQ ID |
| | (SEQ ID | (SEQ ID | NO: 272) |
| | NO: 270) | NO: 271) | |
| N1wc110 | GYNFTGYY | INPNSGG | ATDSFDY (SEQ ID NO: 209) |
| | (SEQ ID | (SEQ ID | |
| | NO: 207) | NO: 208) | |
| N1wc112 | GFTFGDYA | ISGSGSG | AKDQTDVTIRDFDY |
| | (SEQ ID | (SEQ ID | (SEQ ID NO: 275) |
| | NO: 273) | NO: 274) | |
| N1wc137 | GYTFTNYG | ISGYNDN | AFSYYDSSGYFN |
| | (SEQ ID | (SEQ ID | (SEQ ID NO: 278) |
| | NO: 276) | NO: 277) | |
| N1wc140 | GGTFSNYA | IIPIFGG (SEQ | ASGAYYDVMGNYPYSGMDV |
| | (SEQ ID | ID NO: 280) | (SEQ ID NO: 281) |
| | NO: 279) | | |
| N1wc142 | GYSFSRYY | INPGGGT | ARSNYDILAGYGPDAFDF (SEQ ID |
| | (SEQ ID | (SEQ ID | NO: 272) |
| | NO: 270) | NO: 271) | |
| N1wc145 | GYTFTTYG | ISTYNGN (SEQ | ARDARKAFDI (SEQ ID NO: 284) |
| | (SEQ ID | ID NO: 283) | |
| | NO: 282) | | |
| N1wc152 | GFTFSSHW | INEGGSA | VRDQFHSNYD (SEQ ID NO: 287) |
| | (SEQ ID | (SEQ ID | |
| | NO: 285) | NO: 286) | |
| N1wc157 | GYIFSQYP | VDTGNGT | ATNAFDY (SEQ ID NO: 290) |
| | (SEQ ID | (SEQ ID | |
| | NO: 288) | NO: 289) | |
| N1wc158 | GYNFTGYY | INPNSGG | ATDSFDY (SEQ ID NO: 209) |
| | (SEQ ID | (SEQ ID | |
| | NO: 207) | NO: 208) | |
| N1wc160 | GYNFTGYY | INPNSGG | ATDSFDY (SEQ ID NO: 209) |
| | (SEQ ID | (SEQ ID | |
| | NO: 207) | NO: 208) | |
| N1wc175 | GYNFTGYY | INPNSGG | ATDSFDY (SEQ ID NO: 209) |
| | (SEQ ID | (SEQ ID | |
| | NO: 207) | NO: 208) | |
| N1wc191 | GYNFTGYY | INPNSGG | ATDSFDY (SEQ ID NO: 209) |
| | (SEQ ID | (SEQ ID | |
| | NO: 207) | NO: 208) | |
| N1wc99 | GYTFTSYG | ISAYNGN (SEQ | ARLASRAFDI (SEQ ID NO: 293) |
| | (SEQ ID | ID NO: 292) | |
| | NO: 291) | | |
| N1wc14 | GFTSDDYA | ITWNSGH | AKASYLSTSSSLDY |
| | (SEQ ID | (SEQ ID | (SEQ ID NO: 262) |
| | NO: 294) | NO: 295) | |
| N1wc179 | GYNFTGYY | INPNSGG | ATDSFDY (SEQ ID NO: 209) |
| | (SEQ ID | (SEQ ID | |
| | NO: 207) | NO: 208) | |
| N1wc23 | GYNFTGYY | INPNSGG | ATDSFDY (SEQ ID NO: 209) |
| | (SEQ ID | (SEQ ID | |
| | NO: 207) | NO: 208) | |
| N1wc33 | GFTFSDSW | IGIDGIT (SEQ | ARNVGWESRP |
| | (SEQ ID | ID NO: 297) | (SEQ ID NO: 298) |
| | NO: 296) | | |
| N1wc48 | GYTFTDYY | VDPEDGE | ATGPFRLDS (SEQ ID NO: 300) |
| | (SEQ ID | (SEQ ID | |
| | NO: 268) | NO: 299) | |
| N1wc60 | GYNFTGYY | INPNSGG | ATDSFDY (SEQ ID NO: 209) |
| | (SEQ ID | (SEQ ID | |
| | NO: 207) | NO: 208) | |
| N1wc202 | GYNFTGYY | INPNSGG | ATDSFDY (SEQ ID NO: 209) |
| | (SEQ ID | (SEQ ID | |
| | NO: 207) | NO: 208) | |

Light Chain

| Clone Name | LC-CDR1 | LC-CDR2 | LC-CDR3 |
|---|---|---|---|
| hN1S3-067 | SGSIASNY (SEQ | DD | QSYDRFDHVV (SEQ ID |
| | ID NO: 301) | | NO: 302) |
| hN1S3-116 | SSNIGSNT (SEQ | NN | ASWDDSLNGRV (SEQ |
| | ID NO: 226) | | ID NO: 303) |
| hN1S3-073 | QGINNY (SEQ ID | AA | QQYDSYPHT (SEQ ID |
| | NO: 304) | | NO: 305) |
| hN1S3-193 | SSNIGSNT (SEQ | SN | AAWDDSLNGYV (SEQ |
| | ID NO: 226) | | ID NO: 215) |
| N1wc520 | SSNIESNA (SEQ | SH | AAWDDSLSGYV (SEQ |
| | ID NO: 306) | | ID NO: 307) |
| N1wc625 | RSNIGTNY (SEQ | TT | ASWDESLNGVV (SEQ |
| | ID NO: 308) | | ID NO: 309) |
| N1wc103 | SSNIGSNP (SEQ | SN | AAWDDSLNGSSV (SEQ |
| | ID NO: 310) | | ID NO: 311) |
| N1wc106 | SSNIGADND | SN | QSYDSGLTREL (SEQ ID |
| | (SEQ ID NO: 312) | | NO: 313) |
| N1wc110 | SSNIGSNT (SEQ | NN | ATWDDSLIGLA (SEQ ID |
| | ID NO: 226) | | NO: 314) |
| N1wc112 | NLGRKS (SEQ ID | ED | YSTDSSGNHRV (SEQ ID |
| | NO: 315) | | NO: 316) |
| N1wc137 | SSNIGTGYA | GN | QSFDTLSGSRV (SEQ ID |
| | (SEQ ID NO: 317) | | NO: 318) |
| N1wc140 | KIETKS (SEQ ID | ED | YSTDATGTERV (SEQ ID |
| | NO: 319) | | NO: 320) |
| N1wc142 | NSNIGAGHD | DN | QSYDSSLGEV (SEQ ID |
| | (SEQ ID NO: 321) | | NO: 322) |
| N1wc145 | SSDVGGYNY | EG | SSYTTRSTRV (SEQ ID |
| | (SEQ ID NO: 266) | | NO: 323) |
| N1wc152 | SSNIGAPFD | GN | QSYDSSLSGWV (SEQ |
| | (SEQ ID NO: 324) | | ID NO: 325) |
| N1wc157 | ALPDKY (SEQ ID | DD | YSTDSSGNERV (SEQ ID |
| | NO: 326) | | NO: 327) |
| N1wc158 | SSNIGSNS (SEQ | RN | AAWDDSLSGLV (SEQ ID |
| | ID NO: 328) | | NO: 329) |
| N1wc160 | SSNIGSNT (SEQ | TN | ASWDDSLKAYV (SEQ ID |
| | ID NO: 226) | | NO: 330) |
| N1wc175 | RSSIGSNT (SEQ | TN | ASWDDSLNDYV (SEQ |
| | ID NO: 331) | | ID NO: 332) |
| N1wc191 | SSNIGSNI (SEQ | TS | AAWDDSLNAVV (SEQ ID |
| | ID NO: 251) | | NO: 333) |
| N1wc99 | SSDVGGYNY | EG | SSYTTRSTRV (SEQ ID |
| | (SEQ ID NO: 266) | | NO: 267) |
| N1wc14 | NIGSKS (SEQ ID | ED | YSTDSSGNHRV (SEQ ID |
| | NO: 334) | | NO: 316) |
| N1wc179 | SSNIGSNT (SEQ | ND | AAWDDSLNGLA (SEQ ID |
| | ID NO: 226) | | NO: 335) |
| N1wc23 | YSNIGSNY (SEQ | TN | AAWDDSVGVLL (SEQ ID |
| | ID NO: 244) | | NO: 336) |
| N1wc33 | DSNIGRNT (SEQ | TN | AAWDDSLNAYF (SEQ ID |
| | ID NO: 337) | | NO: 338) |
| N1wc48 | SSNIGAGYD | GN | QSFDNRLSDFNV (SEQ |
| | (SEQ ID NO: 241) | | ID NO: 339) |
| N1wc60 | SSNIGSNY (SEQ | RN | AAWDDSLSGLV (SEQ ID |
| | ID NO: 228) | | NO: 329) |
| N1wc202 | SSNIESNA (SEQ | SH | AAWDDSLSGYV (SEQ |
| | ID NO: 306) | | ID NO: 307) |

APPENDIX IV
Group II - Notch1/Notch3 antibodies

A. Aminoacid sequence
Heavy Chain

| Clone Name | HC-sequences |
|---|---|
| hN1S3- | EVQLVQSGAEVKKPGASVKVSCKASGYNFTGYYMHWVRQAPGQGLEWMGWIN |
| 001 | PNSGGTNYAQKFQGRVTMTRDTSISTAYMELSRLRSDDTAVYYCATDSFDYWGQ |
| | GTMVTVSS (SEQ ID NO: 340) |
| hN1S3- | EVQLVQSGPEVKKPGTSVKVSCKASGFTFTNSAVQWVRQARGQRLEWIGWIVVG |
| 003 | SGNTNYAQKFQERVTITRDMSTSTAYMELSSLKSEDTAVYYCVADTVYWGQGTLV |
| | TVSS (SEQ ID NO: 341) |
| hN1S3- | QVQLVQSGAEVKKPGASVKVSCKASGYNFTGYYMHWVRQAPGQGLEWMGWIN |
| 007 | PNSGGTNYAQKFQGRVTMTRDTSISTAYMELSRLRSDDTAVYYCATDSFDYWGQ |
| | GTMVTVSS (SEQ ID NO: 342) |
| hN1S3- | EVQLVQSGAEVKKPGASVKVSCKASGYNFTGYYMHWVRQAPGQGLEWMGWIN |
| 023 | PNSGGTNYAQKFQGRVTMTRDTSISTAYMELSRLRSDDTAVYYCATDSFDYWGQ |
| | GTMVTVSS (SEQ ID NO: 343) |
| hN1S3- | EVQLVQSGAEVKKPGASVKVSCKASGYNFTGYYMHWVRQAPGQGLEWMGWIN |
| 032 | PNSGGTNYAQKFQGRVTMTRDTSISTAYMELSRLRSDDTAVYYCATDSFDYWGK |
| | GTLVTVSS (SEQ ID NO: 344) |
| hN1S3- | QVQLVQSGAEVKKPGASVKVSCKASGYNFTGYYMHWVRQAPGQGLEWMGWIN |
| 035 | PNSGGTNYAQKFQGRVTMTRDTSISTAYMELSRLRSDDTAVYYCATDSFDYWGQ |
| | GTLVTVSR (SEQ ID NO: 345) |
| hN1S3- | EVQLVQSGPEVKKPGTSVKVSCKASGFTFTSSAVQWVRQARGQRLEWIGWIVVG |
| 040 | SGNTNYAQKFQERVTITRDMSTSTAYMELSSLRSEDTAVYYCVADTVYWGRGTTV |
| | TVSS (SEQ ID NO: 346) |
| hN1S3- | EVQLVQSGAEVKKPGASVKVSCKASGYNFTGYYMHWVRQAPGQGLEWMGWIN |
| 053 | PNSGGTNYAQKFQGRVTMTRDTSISTAYMELSRLRSDDTAVYYCATDSFDYWGR |
| | GTTVTVSS (SEQ ID NO: 347) |
| hN1S3- | QVQLVQSGAEVKKPGASVKVSCKASGYNFTGYYMHWVRQAPGQGPEWMGWIN |
| 055 | PNSGGTNYAQKFQGRVTMTRDTSISTAYMELSRLRSDDTAVYYCATDSFDYWGR |
| | GTMVTVSS (SEQ ID NO: 348) |
| hN1S3- | QVQLVQSGPEVKKPGTSVKVSCKASGFTFTSSAVQWVRQARGQRLEWIGWIVVG |
| 147 | SGNTNYAQKFQERVTITRDMSTSTAYMELSSLRSEDTAVYYCVADTVYWGKGTLV |
| | TVSS (SEQ ID NO: 349) |
| hN1S3- | QVQLVQSGPEVKKPGTSVKVSCKASGFTFTSSAVQWVRQARGQRLEWIGWIVVG |
| 082 | SGNTNYAQKFQERVTITRDMSTSTAYMELSSLRSEDTAVYYCVADTVYWGRGTLV |
| | TVSS (SEQ ID NO: 350) |
| hN1S3- | EVQLLESGGGLVQPGGSLRLSCAASGFTFSSYAMSWVRQAPGKGLEWVSAISGS |
| 088 | GGSTYYADSVKGRFTISRDNSKNTLYLQMNSLRAEDTAVYYCATEPPFDIWGRGT |
| | MVTVSS (SEQ ID NO: 351) |
| hN1S3- | EVQLVQSGAEVKKPGASVKVSCKASGYNFTGYYMHWVRQAPGQGLEWMGWIN |
| 096 | PNSGGTNYAQKFQGRVTMTRDTSISTAYMELSRLRSDDTAVYYCATDSFDYWGR |
| | GTTVTVSS (SEQ ID NO: 352) |
| hN1S3- | QVQLVQSGAEVKKPGASVKVSCKASGYNFTGYYMHWVRQAPGQGLEWMGWIN |
| 112 | PNSGGTNYAQKFQGRVTMTRDTSISTAYMELSRLRSDDTAVYYCATDSFDYWGR |
| | GTLVTVSS (SEQ ID NO: 353) |
| hN1S3- | EVQLVQSGAEVKKPGASVKVSCKASGYNFTGYYMHWVRQAPGQGLEWMGWIN |
| 124 | PNSGGTNYAQKFQGRVTMTRDTSISTAYMELSRLRSDDTAVYYCATDSFDYWGR |
| | GTTVTVSS (SEQ ID NO: 354) |
| hN1S3- | EVQLVQSGAEVKKPGASVKVSCKASGYNFTGYYMHWVRQAPGQGLEWMGWIN |
| 127 | PNSGGTNYAQKFQGRVTMTRDTSISTAYMELSRLRSDDTAVYYCATDSFDYWGQ |
| | GTLVTVSS (SEQ ID NO: 355) |
| hN1S3- | QVQLQESGAEVKKPGASVKVSCKASGYNFTGYYMHWVRQAPGQGLEWMGWIN |
| 136 | PNSGGTNYAQKFQGRVTMTRDTSISTAYMELSRLRSDDTAVYYCATDSFDYWGR |
| | GTTVTVSS (SEQ ID NO: 356) |
| hN1S3- | EVQLVQSGAEVKKPGASVKVSCKASGYNFTGYYMHWVRQAPGQGLEWMGWIN |
| 177 | PNSGGANYAQKFQGRVTMTRDTSISTAYMELSRLRSDDTAVYYCATDSFDYWGR |
| | GTLVTVSS (SEQ ID NO: 357) |
| hN1S3- | QVQLVQSGAEVKKPGASVKVSCKASGYNFTGYYMHWVRQAPGQGLEWMGWIN |
| 181 | PNSGGTNYAQKFQGRVTMTRDTSISTAYMELSRLRSDDTAVYYCATDSFDYWGR |
| | GTLVTVSS (SEQ ID NO: 358) |
| hN1S3- | QVQLVQSGAEVKKPGASVKVSCKASGYNFTGYYMHWVRQAPGQGLEWMGWIN |
| 182 | PNSGGTNYAQKFQGRVTMTRDTSISTAYMELSRLRSDDTAVYYCATDSFDYWGR |
| | GTMVTVSS (SEQ ID NO: 359) |
| hN1S3- | QVQLVQSGAEVKKPGASVKVSCKASGYNFTGYYMHWVRQAPGQGLEWMGWIN |
| 184 | PNSGGTNYAQKFQGRVTMTRDTSISTAYMELSRLRSDDTAVYYCATDSFDYWGR |
| | GTTVTVSS (SEQ ID NO: 360) |
| hN1S3- | QVQLVQSGAEVKKPGASVKVSCKASGYNFTGYYMHWVRQAPGQGLEWMGWIN |
| 191 | PNSGGTNYAQKFQGRVTMTRDTSISTAYMELSRLRSDDTAVYYCATDSFDYWGR |
| | GTLVTVSS (SEQ ID NO: 361) |
| hN1S3- | GVQLVQSGAEVKKPGASVKVSCKASGYTFTDYYVHWVRQAPGQGPEWMGWINP |
| 002 | NNGGTKYAQKFQGRVTMTTDTSTSTAYMELRSLRSDDTALYYCARDLLPYYGDY |
| | RADKIDDFDIWGRGTLVTVSS (SEQ ID NO: 362) |
| hN1S3- | QVQLVQSGAEVKKPGASVKVSCKASGYNFTGYYIHWVRQAPGQGLEWMGWINP |
| 006 | NSGGTNYAQKFQGRVIMTRDTSISTAYMELSRLRSDDTAVYYCATDSFDYWGRG |
| | TLVTVSS (SEQ ID NO: 363) |
| hN1S3- | QVQLVQSGAEVKKPGASVKVSCKASGYNFTGYYMHWVRQAPGQGLEWMGWIN |
| 030 | PNSGGTNYAQKFQGRVTMTRDTSISTAYMELSRLRSDDTAVYYCATDSFDYWGQ |
| | GTMVTVSS (SEQ ID NO: 364) |
| hN1S3- | EVQLVQSGAEVKKPGASVKVSCKASGYNFTGYYMHWVRQAPGQGLEWMGWIN |
| 036 | PNSGGTNYAQKFQGRVTMTRDTSISTAYMELSRLRSDDTAVYYCATDSFDYWGR |
| | GTMVTVSS (SEQ ID NO: 365) |
| hN1S3- | EVQLVQSGAEVKKPGASVKVSCKASGYNFTGYYMHWVRQAPGQGLEWMGWIN |
| 038 | PNSGGTNYAQKFQGRVIMTRDTSISTAYMELSRLTSDDTAVYYCATDSFDYWGRG |
| | TTVTVSR (SEQ ID NO: 366) |
| hN1S3- | EVQLVQSGAEVKKPGASVKVSCKASGYNFTGYYMHWVRQAPGQGLEWMGWIN |
| 074 | PNSGGTNYAQKFQGRVTMTRDTSISTAYMELSRLRSDDTAVYYCATDSFDYWGR |
| | GTMVTVSS (SEQ ID NO: 367) |
| hN1S3- | EVQLVQSGAEVKKPGAPVKVSCKTSGYGFSNYYMHWVRQAPGQGLEWLGMINP |
| 077 | SDGSQHFAQRFRDRVSFTSDTSTSAVYMELAGLRSDDTAVYYCAREKANSMYFD |
| | YWGQGTLVTVSS (SEQ ID NO: 368) |
| hN1S3- | QVQLQQSGPGLVKPSQTLSLTCAISGDSISSSSATWNWIRQSPSRGLEWLGRTYY |
| 083 | RSKWYNDYAVSVKSRITIDPDTSKNQFSLQLNSVTPEDTAVYYCARQLGGRIEFW |
| | GQGTLVTVSS (SEQ ID NO: 369) |
| hN1S3- | QVQLVQSGAEVKKPGASVRISCKTSGFTFTSYFIHWVRQAPGQRPEWMGVINPS |
| 087 | RGNADYAPRFRGRVTMTRDKSTHTVYMDLKSLRSDDAAIYYCARDRLGGLLDYY |
| | DSSDSSKNFDAFDFWGQGTLVTVSS (SEQ ID NO: 370) |
| hN1S3- | EVQLVQSGAEVKKPGASVKVSCKASGYNFTGYYMHWVRQAPGQGLEWMGWIN |
| 091 | PNSGGTNYAQKFQGRVTMTRDTSISTAYMELSRLRSDDTAVYYCATDSFDYWGQ |
| | GTMVTVSS (SEQ ID NO: 371) |
| hN1S3- | EVQLVQSGAEVKKPGASVKVSCKASGYNFTGYYMHWVRQAPGQGLEWMGWIN |
| 092 | PNSGGTNYAQKFQGRVTMTRDTSISTAYMELSRLRSDDTAVYYCATDSFDYWGQ |
| | GTLVTVSS (SEQ ID NO: 372) |
| hN1S3- | EVQLVQSGAEVKKPGASVKVSCKASGYNFTGYYMHWVRQAPGRGLEWMGWINP |
| 095 | NSGGTNYAQKFQGRVTMTRDTSISTAYMELSRLRSDDTAVYYCATDSFDYWGQG |
| | TLVTVSS (SEQ ID NO: 373) |
| hN1S3- | EVQLVQSGPEVKKPGASVKVSCKASGYTFTDYFLNWVRQAPGQGLEWIGWINPN |
| 107 | TGGTNYAQNFQGRVTVTRDASISTAYMELRSLTSADTAIYYCARGAYDYVWGSHR |
| | YVADAFDIWGQGTLVTVSS (SEQ ID NO: 374) |
| hN1S3- | QVQLVQSGPEVKKPGTSVKVSCKASGFTFTSSAVQWVRQARGQRLEWIGWIVVG |
| 133 | SGNTNYAQKFQERVTITRDMSTSTAYMELSSLRSEDTAVYYCVADTVYWGQGTLV |
| | TVSS (SEQ ID NO: 375) |
| hN1S3- | EVQLVQSGAEVKKPGASVKVSCKASGYNFTGYYMHWVRQAPGQGLEWMGWIN |
| 135 | PNSGGTNYAQKFQGRVTMTRDTSISTAYMELSRLRSDDTAVYYCATDSFDYWGK |
| | GTLVTVSS (SEQ ID NO: 376) |
| hN1S3- | QVQLVQSGAEVKKPGASVKVSCKASGYNFTGYYIHWVRQAPGQGLEWMGWINP |
| 141 | NSGGTNYAQKFQGRVIMTRDTSISTAYMELSRLRSDDTAVYYCATDSFDYWGRG |
| | TLVTVSS (SEQ ID NO: 377) |
| hN1S3- | QVQLVQSGAEVKKPGASVKVSCKASGYNFTGYYMHWVRQAPGQGLEWMGWIN |
| 151 | PNSGGTNYAQKFQGRVTMTRDTSISTAYMELSRLRSDDTAVYYCATDSFDYWGR |
| | GTLVTVSS (SEQ ID NO: 378) |
| hN1S3- | QVQLQESGGGLVQPGGSLSLSCATSGFTFGNYVMRWVRQAPGKGLEWVSTITE |
| 153 | GGGDFIYYADSVKGRFAISRDNSKSTVYLHMNGLRVEDTAVYYCAKSGGDDNSNY |
| | NFEYWGQGTLVTVSS (SEQ ID NO: 379) |
| hN1S3- | QVQLVQSGAEVKKPGASVRISCKTSGFTFTSYFIHWVRQAPGQRPEWMGVINPS |
| 190 | RGNADYAPRFRGRVTMTRDKSTHTVYMDLKSLRSDDAAIYYCARDRLGGLLDYY |
| | DSSDSSKNFDAFDFWGQGTLVTVSS (SEQ ID NO: 380) |
| hN1S3- | QVQLVQSGAEVRKPGASVKVSCKASGYTFTDYYLHWVRQAPGLGLEWMGWINP |
| 108 | NTGGTDYGQKFQGRVTMTRDTSINTAYMELSSLRSDDTAVYYCARGAYDYVWGS |
| | HRYVADAFDIWGQGTTVTVSS (SEQ ID NO: 381) |
| hN1S3- | KVQLVQSGAEVKKPGASVKVSCKASGYTFTDFYIHWVRQAPGQGLEWMGWINSK |
| 130 | SGDTHYAQKFQGRVTLTRDTSTDTAYMELSRLTSDDTAVYYCARGAYDYVWGSS |
| | RYVADAFDIWGRGTMVTVSS (SEQ ID NO: 382) |
| N1wc11 | EVQLVQSGPEVKKPGTSVKVSCKASGFTFTNSAVQWVRQARGQRLEWIGWIVVG |
| | SGNINYAQKFQERVTITRDMSTSTAYMELSSLKSEDTAVYYCVADTVYWGRGTMV |
| | TVSS (SEQ ID NO: 383) |
| N1wc13 | QVQLVQSGAEVKKPGASVKVSCKASGYNFTGYYMHWVRQAPGQGLEWMGWIN |
| | PNSGGTNYAQKFQGRVTMTRETSISTAYMELSRLRSDDTAVYYCATDSFDYWGR |
| | GTMVTVSS (SEQ ID NO: 384) |
| N1wc15 | QVQLQQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGQ |
| | GTMVTVSS (SEQ ID NO: 385) |
| N1wc16 | QVQLQQSGAEVKTPGASVKVSCKASGYTFTDNYIHWVRQAPGQGLEWMGRINV |
| | NSGATNYARKFQGRVTMTGDTSISTAYMDLSRLKSDDTAVYYCARDFRYSGVDV |
| | WGQGTMVTVSS (SEQ ID NO: 386) |
| N1wc21 | QVQLVESGAEVKKPGASVKVSCKASGYTFTDHYIQWLRQAPGQGLEWMGWMNP |
| | KSGGTNYAQKFQGRVTLTRDTSISTAYMDLSGLRFDDTAIYFCASDPTIPPSPNYD |
| | YYYYAMDLWGKGTLVTVSS (SEQ ID NO: 387) |
| N1wc24 | EVQLVESGPGLVKPSETLSLTCTVSGGSMTSNFWSWIRQTPGKGLEWIGYVLYTG |
| | STNSNPSLKSRVTMSVDTSKNQVSLTLSSVTAADTAIYYCARGNGWYLPWGRGT |
| | MVTVSS (SEQ ID NO: 388) |
| N1wc72 | EVQLVQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGK |
| | GTMVTVSS (SEQ ID NO: 389) |
| N1wc79 | QVQLQQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGQ |
| | GTMVTVSS (SEQ ID NO: 390) |
| N1wc82 | QVQLVQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGK |
| | GTLVTVSS (SEQ ID NO: 391) |
| N1wc102 | QVQLVQSGAEVKKPGAPVKVSCKASGYNFTGYYMHWVRQAPGQGLEWMGWIN |
| | PNSGGTNYAQKFQGRVAMTRDTSISTAYMELSRLRSDDTAVYYCATDSFDYWGQ |
| | GTMVTVSS (SEQ ID NO: 392) |
| N1wc113 | QMQLVQSGAEVKRPGASVNLSCKASGYSFSRYYIHWVRQAPGQGLELMGRINPG |
| | GGTTTYAQKFQGRVTLSRDTSTNTIFMELNGLTSEDTAMYYCARSNYDILAGYGP |
| | DAFDFWGQGTMVTVSS (SEQ ID NO: 393) |
| N1wc117 | QVQLVQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGR |
| | GTLVTVSS (SEQ ID NO: 394) |
| N1wc125 | QVQLQQSGAEVKKPGAPLKVSCKTSGFTFTGYYIHWVRQAPGQGLEWMGRVDP |
| | YNGATNFAQKFQGRVTMTTDTSTSTTYMELMSLRSDDTAVYYCARGWELDSWG |
| | QGTLVTVSS (SEQ ID NO: 395) |
| N1wc127 | QVQLVQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGR |
| | GTLVTVSS (SEQ ID NO: 396) |
| N1wc129 | QVQLVQSGAEVKKPGASVKVSCKASGYNFTGYYLHWVRQAPGQGLEWMGWIDP |
| | NSGGTDYAQQFQGRVTVTRDTSISTAYMELSGLRSDDTAVYYCASDSFDHWGQG |
| | TLVTVSS (SEQ ID NO: 397) |
| N1wc130 | QVQLQQSGAEVKKPGASVKVSCKASGYNFTGYYMHWVRQAPGQGLEWMGWIN |
| | PNSGGTNYAQKFQGRVTMTRDTSISTAYMELSRLRPDDTAVYYCATDSFDYWGK |
| | GTMVTVSS (SEQ ID NO: 398) |
| N1wc131 | EVQLVQSGAEAKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGQ |
| | GTLVTVSS (SEQ ID NO: 399) |
| N1wc132 | QVQLVQSGAEVKKPGASVKVSCKASGYNFTGYYLHWVRQAPGQGLEWMGWINP |
| | NSGGTDYAQKFQGRVTVTRDTSISTAYMELGGLRSDDTAVYYCVSDSFDHWGRG |
| | TLVTVSS (SEQ ID NO: 400) |
| N1wc135 | QVQLVQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGQ |
| | GTLVTVSS (SEQ ID NO: 401) |
| N1wc150 | EVQLVQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGR |
| | GTMVTVSS (SEQ ID NO: 402) |
| N1wc164 | QVQLVQSGADVKKPGASVKISCKASGYTFTKYYMHWVRQAPGQGLEWMGIINPT |
| | NGYTSYAQKFQGRVTMTSDTSASTVSMELSSLRSEDTAVYYCTRSYSNYDSFDIW |
| | GRGTMVTVSS (SEQ ID NO: 403) |
| N1wc170 | QVQLQQSGPEVKKPGASVKISCKTSGYTFSSFGISWVRQAPGQGPEWMGWVSP |
| | YSGDTDYAQKFRGRVTMTIDSSTDTAYVEMRSLRSDDTAIYYCANSPISDGSSSSY |
| | DNWGKGTLVTVSS (SEQ ID NO: 404) |
| N1wc177 | QVQLVQSGAEVKEPGSSVRVSCKSSGGTFSNYAISWVRQAPGQGLEWMGGIIPG |
| | FGKTNYAQKFQGRVTITADASTSTTYMELRTLRSGDTAVYYCARDRGPDIGVAHF |
| | DSWGKGTMVTVSS (SEQ ID NO: 405) |
| N1wc182 | QVQLVQSGPEVKKPGTSVKVSCKASGFTFTSSAVQWVRQARGQRLEWIGWIVVG |
| | SGNTNYAQKFQERVTITRDMSTSTAYMELSSLRSEDTAVYYCVADTVYWGQGTLV |
| | TVSS (SEQ ID NO: 406) |
| N1wc197 | EVQLVQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGR |
| | GTLVTVSS (SEQ ID NO: 407) |
| N1wc225 | QVQLQQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGR |
| | GTMVTVSS (SEQ ID NO: 408) |
| N1wc239 | EVQLVESGAEVKKPGASVKVSCKASGYNFTGYYIHWVRQAPGQGLEWMGWINP |
| | NSGGTNYAQKFQGRVIMTRDTSISTAYMELSRLRSDDTAVYYCATDSFDYWGKGT |
| | MVTVSS (SEQ ID NO: 409) |
| N1wc241 | EVQLVQSGAEVKKPGASVKVSCKTSGYTFNDYYHHWVRQAPGQGLEWMGWINP |
| | HTGVANYAEKYKGRLAMTGDTSISTIYMDLSSLISDDTAIYFCARERSEWRNTVSS |
| | PSEYFHHWGKGTLVTVSS (SEQ ID NO: 410) |
| N1wc248 | EVQLVQSGAEVKKPGASVKVSCKASGYNFTGYYMHWVRQAPGQGLEWMGWIN |
| | PNSGGTNYAQKFQGRVTMTRDTSISTAYMELSRLRSDDSAVYYCATDSFDYWGR |
| | GTLVTVSS (SEQ ID NO: 411) |
| N1wc266 | QVQLVQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGQ |
| | GTMVTVSS (SEQ ID NO: 412) |
| N1wc281 | QVQLQESGAEVKKPGASVKVSCRASGYTFTTNSLHWVRQAPGQGLDWMGVINP |
| | RDGSATVAQKFQGRITMTSDTSTSTVFLALSSLTSDDTGVYFCARDGLDYRDTSRI |
| | LAPTDVWGRGTLVTVSS (SEQ ID NO: 413) |
| N1wc289 | QVQLVQSGAEVKKPGTTVKISCKVSGYKFTDYYIHWVRQAPGKGLEWMGFVDPT |
| | DDETRFAEKFQGRLTMTADTSRDTASMELSSLRSEDTAVYYCIRGIAALADWGRG |
| | TMVTVSS (SEQ ID NO: 414) |
| N1wc295 | QVQLQQSGAEVKKPGASVKVSCKASGYNFTGYYMHWVRQAPGQGLEWMGWIN |
| | PNSGGTNYAQNFQGRVTMTRDTSISTAYMELSRLRSDDTAVYYCATDSFDYWGR |
| | GTMVTVSS (SEQ ID NO: 415) |
| N1wc301 | QVQLQQSGAEVKKPGASVKVSCKASGYIFGTYAIHWVRQAPGQSLEWMGWINAG |
| | TGNTKYSQKFQGRVTITMDTSATTAYMELSSLRSGDTAVYYCTRGDYYDSRGYYN |
| | RFDYWGRGTLVTVSS (SEQ ID NO: 416) |
| N1wc319 | QVQLVQSGAEVEKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGR |
| | GTLVTVSS (SEQ ID NO: 417) |
| N1wc336 | QVQLVQSGAEVKRPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGK |
| | GTLVTVSS (SEQ ID NO: 418) |
| N1wc338 | QVQLVQSGAEVKKPGASVKVSCKASGYNFTGYYMHWVRQAPGQGLEWMGWIN |
| | PNSGGTNYAQKF*GRVTMTRDTSISTAYMELSRLRSDDTAVYYCATDSFDYWGKG |
| | TLVTVSS (SEQ ID NO: 419) |
| N1wc403 | EVQLVQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGR |
| | GTLVTVSS (SEQ ID NO: 420) |
| N1wc441 | QVQLVQSGAEVKKPGASVKVSCKAPGYSFTDYYLHWVRQAPGIGLEWVGWINPN |
| | NGGTNYAQKFRGRVTMTRDTAINTAYMDMRWLRSDDTAVYYCATDGPPGTGNA |
| | FDIWGRGTLVTVSS (SEQ ID NO: 421) |
| N1wc476 | EVQLVQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGQ |
| | GTLVTVSS (SEQ ID NO: 422) |
| N1wc516 | EVQLVESGAEVKKPGASVKVSCKASGYNFTGYYMHWVRQAPGQGLEWMGWINP |
| | NSGGTNYAQKFQGRVIMTRDTSISTAYMELSRLTSDDTAVYYCATDSFDYWGRGT |
| | LVTVSS (SEQ ID NO: 423) |
| N1wc535 | QVQLVQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGR |
| | GTLVTVSS (SEQ ID NO: 424) |
| N1wc540 | EVQLVQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGQ |
| | GTMVTVSS (SEQ ID NO: 425) |
| N1wc558 | QVQLVQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGR |
| | GTLVTVSS (SEQ ID NO: 426) |
| N1wc570 | QVQLVQSGPEVKKPGTSVKVSCKASGFTFTSSAVQWVRQARGQRLEWIGWIVVG |
| | SGNTNYAQKFQERVTITRDMSTSTAYMELSSLRSEDTAVYYCVADTVYWGRGTM |
| | VTVSS (SEQ ID NO: 427) |
| N1wc572 | QVQLVQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGR |
| | GTLVTVSS (SEQ ID NO: 428) |
| N1wc586 | QVQLVQSGPEVKKPGASVKVSCKAAGYTFPDFYMHWVRQAPGKGPEWVGWINS |
| | NSGSTRLAERLEGRVTLTRDTSISTAYMELTSLTSDDTAVYYCARGAYDYVWGTS |
| | RYVADAFDIWGQGTLVTVSP (SEQ ID NO: 429) |
| N1wc614 | EVQLVQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGQ |
| | GTLVTVSS (SEQ ID NO: 430) |
| N1wc621 | EVQLVQSGAEVKKPGASVKVSCKASGYTFTGYYIHWVRQAPGQGLEWMGWISP |
| | NSGVTNYAQRFQGRVTMSRDTSITTAYMELNRLGSDDTAVYYCAREGREDGVLF |
| | DYWGRGTLVTVSS (SEQ ID NO: 431) |
| N1wc624 | QVQLQQSGAEVKKPGASVKVSCKASGYNFTGYYMHWVRQAPGQGLEWMGWIN |
| | PNSGGTNYAQKFQGRVTMTRDTSISTAYMELSRLGSDDTAVYYCATDSFDYWGR |
| | GTLVTVSS (SEQ ID NO: 432) |
| N1wc633 | QVQLQQSGAEVKKPGASVKVSCKASGYNFTGYYMHWVRQAPGQGLEWMGWIN |
| | PNSGGTNYAQKFQGRVTMTRDTSISTAYMELSRLRSDDTAVYYCATDSFDYWGR |
| | GTLVTVSS (SEQ ID NO: 433) |
| N1wc45 | EVQLVQSGAEVKKPGASVKVSCKASGYSFRSYGIVWVRQAPGQGLEWMGWISA |
| | YNGNTNYAQKLQGRVTMTTDTSTSTAYMELRSLRSDDTAVYYCVRGDRHYDILTG |
| | YSPVWFDPWGKGTTVTVSS (SEQ ID NO: 434) |
| N1wc39 | EVQLVQSGAEVKKPGASVKLSCKTSGHTFTNYAFHWLRQAPGQRPEWLGWINAD |
| | NGNTKYSQSFQDRVTITRDISANTAYMEMSRLKSEDTAYYYCARDPWSGVLDHW |
| | GKGTLVTVSS (SEQ ID NO: 435) |

Light Chain

| Clone Name | LC amino acid sequences |
|---|---|
| hN1S3- | QSVVTQPPSASGTPGQRVTISCSGSSSNIGSNTVNWYQQLPGTAPKLLIYSNNQR |
| 001 | PSGVPDRFSGSKSGTSASLAISGLQSEDEADYYCAAWDDSLNGYVFGTGTKLTVL |
| | (SEQ ID NO: 436) |
| hN1S3- | QSVLTQPPSASGTPGQRVTISCSGSSSNIGSNTVNWYQQLPGTAPKLLIYSNNQR |
| 003 | PSGVPDRFSGSKSGTSASLAISGLQSEDEADYYCAAWDDSLNGWVFGGGTKLTV |
| | L (SEQ ID NO: 437) |
| hN1S3- | QAVLTQPSSVSGTPGQRVTMSCSGGRSNIGSNAVNWYQQFPGTAPKLLMYNTN |
| 007 | QRPSGVPGRFSGSKSGASASLAISGLQSDDEADYYCSSWDDSLNGLEFGGGTKL |
| | TVL (SEQ ID NO: 438) |
| hN1S3- | QSVVTQPPSASGTPGQRVTISCSGSSSNIGSNTVNWYQQLPGTALKLLIYSNNQR |
| 023 | PSGVPDRFSGSKSGTSASLAISGLQSEDEADYYCAAWDDSLNGYVFGTGTKLTVL |
| | (SEQ ID NO: 439) |
| hN1S3- | QAVLTQPSSVSGAPGQRVTISCTGSSSNIGAGYDVHWYRQLPGTAPKLLLYGNDN |
| 032 | RPSGVPDRFSGSKSGASASLAITGLRADDEADYYCQSYDSSLSGSVFGGGTKVTV |
| | L (SEQ ID NO: 440) |
| hN1S3- | QAVLTQPSSVSGAPGQRVTISCTGSSSNIGAGYDVHWYQQLPGTAPKLLIYGNSN |
| 035 | RPSGVPDRFSGSKSGTSASLAITGLQAEDEADYYCQSYDSSLSGSVFGGGTKVTV |
| | L (SEQ ID NO: 441) |
| hN1S3- | QSVLTQPPSASGTPGQGVTISCSGSSSNIGRNSVNWYQQLPGTAPKLLIYGDNQR |
| 040 | PSGVPDRFSGSKPGTSASLAISGLQSDDEAHYYCAAWDDSLNGWVFGGGTKVTV |
| | L (SEQ ID NO: 442) |
| hN1S3- | QAVLTQPSSVSGAPGQRVTISCTGSRSNIGAGYDVHWYQQLPGTAPKLLIYGNSN |
| 053 | RPSGVPDRFSGSKSGTSASLAITGLQAEDEADYYCQSYDSSLSGSVFGGGTKLTV |
| | L (SEQ ID NO: 443) |
| hN1S3- | QSVLTQPPSASGTPGQRVTISCSGSSSNIGSNTVTWYQQVPGTAPKVLIYTNIQRP |
| 055 | SGVPDRFSGSKSGTSASLAISGLQSEDESDYFCAAWDDSLNGVVFGGGTKLTVL |
| | (SEQ ID NO: 444) |
| hN1S3- | SYELTQPPSASGTPGQRVTISCSGSSSNIGSNPVNWYQHLPGTAPKLLIYSDSQRP |
| 147 | SGVPDRFSGSKSGTSASLAISGLQSEDEADYYCATWDDSLSGWVFGGGTKLTVL |
| | (SEQ ID NO: 445) |
| hN1S3- | HVILTQPPSASGTPGQRVTISCSGGGSNIGSNTVNWYQHLPGTAPKLLIYNNDQRP |
| 082 | SGVPDRFSGSKSGTSASLAISGLQSEDEADYYCAAWDDSLNGWLFGGGTKLTVL |
| | (SEQ ID NO: 446) |
| hN1S3- | QSVLTQPPSVSGTPGQRVTISCSGSSSNIGNDVVNWYQQLPGTAPKLLIYNDYQR |
| 088 | PSGVSDRFSGSKSGTSAYLAISGLQSEDEADYHCAVWDDSLDGWVFGGGTKLTV |
| | L (SEQ ID NO: 447) |
| hN1S3- | QPVLTQPPSASGTPGQRVTISCSGSSSNIASNSVYWYQQLPGTAPKLLIYRNNQR |
| 096 | PSGVPDRFSGSQSGASASLAISGLRSEDEADYYCAAWDDNLYGVVFGGGTKLTV |
| | L (SEQ ID NO: 448) |
| hN1S3- | LPVLTQPPSASGTPGQRVTISCSGSSSNIGNNYVYWYQQLPGTAPKLLIYRNNQR |
| 112 | PSGVPDRFSASKSGTSASLVISGIRSEDEAEYYCGGWDDSLSGQVVFGGGTKLTV |
| | L (SEQ ID NO: 449) |
| hN1S3- | QAVLTQPSSASGTPGQRVTISCSGSSSNIGSNTVNWYQQLPGTAPKLLIYATNQR |
| 124 | PSGVPDRFSGSESGTSASLAIRGLQSEDEADYYCAAWDDSLNGKVFGGGTKVTV |
| | L (SEQ ID NO: 450) |
| hN1S3- | QSVLTQPPSVSGAPGQRVTISCSGSTSNIGGNPVTWYQQFPGTAPKLLVYRDNQ |
| 127 | RPSGVADRFSGSRSGTSASLAISGLQSEDEADFYCASWDDSLGGVVFGGGTKLT |
| | VL (SEQ ID NO: 451) |
| hN1S3- | QAVLTQPSSESGTPGQRVTISCSGTTSNIGDNTVTWYQQLPGTAPKLLIYSNTQRP |
| 136 | SGVPARFSGSKSGTSASLAISGLQSGDEADYYCAAWDDSRNGYVFGTGTKLTVL |
| | (SEQ ID NO: 452) |
| hN1S3- | QAVLTQPSSASGTPGQRVTISCSGSSSNIGTNYVYWYQHLPGMAPKLLLYSNNQR |
| 177 | PSGVPDRFSGSKSGPSASLAISGLRSEDEADYYCAAWDDSLSGLVFGGGTKLTVL |
| | (SEQ ID NO: 453) |
| hN1S3- | SYVLTQPPSASGTPGQRVTISCSGSSPNIGSNTVNWYQQLPGTAPKLLIYTNNQRP |
| 181 | SGVPDRFSGSKSATSASLAISGLQFDDEADYFCAAWDDSLLGVVFGGGTKVTVL |
| | (SEQ ID NO: 454) |
| hN1S3- | QAVLTQPSSVSGAPGQRVTISCTGSSSNIGSPYDVNWYQQFPGTAPKLLIYGNINR |
| 182 | PSGVPDRFSGSKSGTSASLAITGLQAEDEADYYCQTYDSTLNGAVFGGGTKLTVL |
| | (SEQ ID NO: 455) |
| hN1S3- | QSVLTQPPSASGTPGQRVTISCSGSSSNIGGNYVYWYQQLPGTAPKLLIYRSNQR |
| 184 | PSGVPDRFSGSKSGTSASLAISGLRSEDEADYYCAAWDDSLHGVIFGGGTKLTVL |
| | (SEQ ID NO: 456) |
| hN1S3- | QAVLTQPSSASGTPGQRVTISCPGSSSNIGSNTVNWYQQLPGTAPKLLIYTNNQR |
| 191 | PSGVPDRFSASKSGTSASLAISGLQSEDEADYYCAAWDDSLNGYVFGTGTKLTVL |
| | (SEQ ID NO: 457) |
| hN1S3- | QSVLTQPPSASGTPGQRVTISCSGSSSNIGSNTVNWYQRLPGAAPQLLIYNNDQR |
| 002 | PSGIPDRFSGSKSGTSGSLVISGLQSEDEADYYCASWDDSLNGRVFGGGTKLTVL |
| | (SEQ ID NO: 458) |
| hN1S3- | QAVLTQPSSASGTAGQRVTISCSGSNSNIGSNTVNWYQQLPGTTPKLLIYSNNER |
| 006 | PSGVPDRFSGSKSGTSASLAISGLQSDDEADYYCAAWDDSLNGYVFGTGTKVTVL |
| | (SEQ ID NO: 459) |
| hN1S3- | QSVLTQPPSASGTPGQRVSISCSGSSSNIGSNYVYWYQQLPGTAPKVLVYRNNQ |
| 030 | RPSGVSDRFSGSKYGTSASLAISGLRSEDEADYYCGSWDDSLSGVVFGGGTKVT |
| | VL (SEQ ID NO: 460) |
| hN1S3- | QSVLTQPPSASGTPGQRVTISCSGSSSNIGSNYVYWYQQLPGTAPKLLIYRNNQR |
| 036 | PSGVPDRFSGSKSGTSASLAISGLRSEDEADYYCAAWDDSLSGVVFGGGTKVTVL |
| | (SEQ ID NO: 461) |
| hN1S3- | QSVVTQPPSASGTPGQRVTISCSGSSSNIGSNYVYWYQQLPGTAPKLLIYRNNQR |
| 038 | PSGVPDRFSGSKSGTSASLAISGLRSEDEADYYCAAWDDSLSGVVFGGGTKLTVL |
| | (SEQ ID NO: 462) |
| hN1S3- | QSVLTQPPSASGTPGQRVTISCSGGSSNIGSNTVTWYQQLPGKAPKLLIYISDQRP |
| 074 | SGVPDRFSGSKSGTSASLAIRGLQSEDEADYYCAAWDDSLNGAVFGTGTKVTVL |
| | (SEQ ID NO: 463) |
| hN1S3- | QSVVTQPPSLSAAPGQRVTISCSGSHSNIGNFYVSWYQQVPGAAPKLLIYDNNER |
| 077 | PSGIPDRFSGSKSGTSATLDITGLQTGDEADYYCSTWDRSLSASVFGSGTKLTVL |
| | (SEQ ID NO: 464) |
| hN1S3- | QSVLTQPPSASGSPGRSVTISCTGTSRDVGAYNYVSWYQQHPGEAPKLLISEVTK |
| 083 | RPSGVPARFSASKSGNTASLTISGLQAEDEGDYYCSSYAGSDSWVFGGGTKLTVL |
| | (SEQ ID NO: 465) |
| hN1S3- | NFMLTQPHSVSESPGKTVTISCTRSSGSIGSSYVQWYQQRPGTSPVTIIYEDSQRP |
| 087 | SGVPDRFSGSIDNSANSASLTISGLKTEDEADYYCQSYDANNRVFGGGTKLTVL |
| | (SEQ ID NO: 466) |
| hN1S3- | QSVLTQPPSATGTPGQTVSISCSGGNSNIGSNYVSWYQQLPGTAPKLLIYSNNQR |
| 091 | PSGVPDRFSGSKSGTSASLAISGLQSEDEADYYCAAWDDSLNGHIFGTGTKLTVL |
| | (SEQ ID NO: 467) |
| hN1S3- | QSVLTQPPSASGTPGQRVTISCSGSSSNIGANYVYWYQQLPGTAPKLLIYKNDQR |
| 092 | PSGVPDRFSGSKSGTSASLAISGLRSEDEADYYCAAWDDSLSGLVFGGGTKVTVL |
| | (SEQ ID NO: 468) |
| hN1S3- | QSVLTQPPSASGTPGQRVTISCSGSNSNIGSNTVNWYQQLPGTAPKVLIYGNLQR |
| 095 | PSGVPDRFSGSKSGTSASLAISGLQSEDEADYYCATWDDSLNGFVFGTGTKLTVL |
| | (SEQ ID NO: 469) |
| hN1S3- | QSVLTQPPSASGTPGQRVTISCSGSSSNIGSNTVNWYQRLPGAAPQLLIYNNDQR |
| 107 | PSGIPDRFSGSKSGTSGSLVISGLQSEDEADYYCASWDDSLNGRVFGGGTKLTVL |
| | (SEQ ID NO: 470) |
| hN1S3- | QSVLTQPPSVSEAPRQRVTISCSGSSSNIGNNAVNWYQQLPGEAPKLLIYYDDLLP |
| 133 | SGVSDRFSGSKSGSSASLAISGLQAEDEADYYCATWDDSLNAWVFGGGTKVTVL |
| | (SEQ ID NO: 471) |
| hN1S3- | LPVLTQPPSVSEAPRQRVTISCSGSSFNIGNNAVNWYQQLPGKAPKLLIYYDDLLP |
| 135 | SGVSDRFSGSKSGTSASLAISGLQSEDEADYYCAAWDDSLNAVVFGGGTKVTVL |
| | (SEQ ID NO: 472) |
| hN1S3- | QSVLTQPPSASGTPGQRVTISCSGSYSNIGSNYVYWYQQFPGTAPKLLIYTNNQR |
| 141 | PSGVPDRFSGSKSGSSASLAISGLRSEDEADYYCAAWDDSVGVLLFGGGTKLTVL |
| | (SEQ ID NO: 473) |
| hN1S3- | QSVVTQPPSASGTPGQRVTISCSGSSSNIGSNTVNWYQQLPGTAPKLLIYSNNQR |
| 151 | PSGVPDRFSGSKSGTSASLAISGLQSEDEADYYCAAWDDSLNGGVFGGGTKLTV |
| | L (SEQ ID NO: 474) |
| hN1S3- | QAVLTQPSSVSGAPGQRVIISCTGTSSNTGAGYYVNWYQQLPGAAPKVLIFGNDN |
| 153 | RPSGVPDRFSGAKSGTSASLAITGLQAEDEADYYCQSYDRSLSGWVFGGGTKLT |
| | VL (SEQ ID NO: 475) |
| hN1S3- | SSELTQDPAVSVALGQTVRITCQGDSLRSNYPSWYQQKPGQAPLLVIYSENRRPS |
| 190 | GIPDRFSASRSGNTASLTITGAQAEDEADYYCHSRGSSGNHKVFGGGTQLTVL |
| | (SEQ ID NO: 476) |
| hN1S3- | DIVMTQSPSTLSASVGDRVTITCRASQGISSWLAWYQQKPGRAPKVLIYKASTLES |
| 108 | GVPSRFSGSGSGTDFTLTISSLQPEDFATYYCQQSYSTPWTFGQGTKLEIK (SEQ |
| | ID NO: 477) |
| hN1S3- | DIVMTQSPSTLSASVGDRVTITCRASQGISSWLAWYQQKPGRAPKVLIYKASTLES |
| 130 | GVPSRFSGSGSGTDFTLTISSLQPEDFATYYCQQSYSTPWTFGQGTKLEIK (SEQ |
| | ID NO: 478) |
| N1wc11 | QPVLTQPPSASGTPGQRVTISCSGSSSNLGINTVNWYQQVPGTAPKLLIYSDHQR |
| | PSGVPDRFSGSKSATSASLAISGLQSEDEADYYCAAWDDSLNGWVFGGGTKLTV |
| | L (SEQ ID NO: 479) |
| N1wc13 | QSVLTQPPSASGTPGQRVTIPCSGSSSNIGSNYVYWYQQLPGTAPKLLIYRNNQR |
| | PSGVPDRFSGSKSGTSASLAISGLRSEDEADYYCAAWDDSLSGLVFGGGTKVTVL |
| | (SEQ ID NO: 480) |
| N1wc15 | SYVLTQPPSASGTPGQRVTVSCSGSSSSIGSNTVNWYQQLPGTAPKLLIYSNNQR |
| | PSGVPDRFSGSKSGTSASLAISGLQSEDEADYYCAAWDDSLNGLVFGGGTKLTVL |
| | (SEQ ID NO: 481) |
| N1wc16 | QSVLTQPPSVSVAPGQTATLSCVGDAIERKSVHWYQQRPGQAPVLVVHDDSDRP |
| | SGIPDRISGSNSGNTATLTITRVEVGDEADYFCQVWDTSSGHDAVVFGGGTKLTVL |
| | (SEQ ID NO: 482) |
| N1wc21 | SYELTQPPSVSVSPGQTARITCSGDALPDKYVHWYQQKSGQAPVLVLFDDSKRPS |
| | GIPERFSGSSSGTVATLIISGAQVEDEGDFFCYSTDSSGNERVFGGGTKLTVL |
| | (SEQ ID NO: 483) |
| N1wc24 | QSVLTQPPSVSGAPRQRVTISCSGSSSNIENNIVNWYQQLPGEAPKLLIYYDNLLP |
| | SGVSDRFSGSKSGTSASLAISGLQSGDEADYYCAAWDDSLKAWFGGGTKLTVL |
| | (SEQ ID NO: 484) |
| N1wc72 | QPVLTQPPSASGTPGQRVTISCSGSYSNIGSNYVYWYQQFPGTAPKLLIYTNNQR |
| | PSGVPDRFSGSKSGSSASLAISGLRSEDEADYYCAAWDDSVGVLLFGGGTKVTVL |
| | (SEQ ID NO: 485) |
| N1wc79 | QSVLTQPPSASGTPGQRVTISCSGSTSNIGKNTVNWYQQLPATAPKLLIYSDNQRP |
| | SGVPDRFSGSKSGTSASLAISGLQSEDESDYYCAAWDDSLDGLVFGGGTKLTVL |
| | (SEQ ID NO: 486) |
| N1wc82 | QSVLTQPPSVSATPGQKVTISCSGSNSNIGNNYVSWYQQLPGTAPKLLIYDSDQR |
| | PAGIPDRFSGSKLGTSATLGITGLQTGDEADYYCATWDAGLSVLIFGGGTKLTVL |
| | (SEQ ID NO: 487) |
| N1wc102 | QPVLTQPPSASGTPGQRVAISCSGSSSNIGSNPVNWYQQLPGTAPKLLIYGDNQR |
| | PSGVPDRFSGSKSGTSASLAVSGLQSEDEADYFCAAWDDSLNGVVFGGGTKLTV |
| | L (SEQ ID NO: 488) |
| N1wc113 | QAVLTQPSSVSGAPGQRVTISCTGSSSNIGAGNDVHWYRQFPGTAPKLLIYGNNK |
| | RPSGVSDRFSGSKSGTSASLAITGLQVEDEADYYCQSYDSSLGGSDVFGGGTKLT |
| | VL (SEQ ID NO: 489) |
| N1wc117 | QSVLTQPPSASGTPGQRVTISCSGSSSNIGGNTVNWYQQLPGTAPKLLIYGTNQR |
| | PSGVPDRFSGSKSGTSASLAISGLQSEDEADYYCAAWDDFLNGKVFGGGTKLTVL |
| | (SEQ ID NO: 490) |
| N1wc125 | QSVLTQPASVSGSPGQSITISCTGTSSDVGGYNYVSWYQQHPGKAPKLMIYEGSK |
| | RPSGVSNRFSGSKSGNTASLTISGLQAEDEADYYCSSYTTRSTRVFGGGTKLTVL |
| | (SEQ ID NO: 491) |
| N1wc127 | SYVLTQPPSTSGAPGQRVTISCSGSSSNIGSNGVNWYHHLPGTAPKLLIYSNFHRP |
| | SGVPDRFSGSKSGTSATLAISGLQSEDEADYYCAAWDDSLNDYVFGVGTKVTVL |
| | (SEQ ID NO: 492) |
| N1wc129 | QSVLTQPPSASGTPGQRATISCSGSGSNIGRNAVNWFQQLPGAAPKLLIYSNIHRP |
| | SGVPDRISGSKSGTSATLAISGLQSEDEADYYCATWDDSLNDYVFGTGTKLTVL |
| | (SEQ ID NO: 493) |
| N1wc130 | QAVLTQPSSVSGAPGQRVTISCTGTGSNIGAGYDVHWYQQLPGTAPKLLIYANTN |
| | RPSGVPDRFSGSKSGTSASLAITGLQAEDEADYYCQSYDSSLSGSVFGGGTKVTV |
| | L (SEQ ID NO: 494) |
| N1wc131 | SYELTQPPSASGTPGQRVTISCSGTSSNIGINYVYWYQQLPRTAPKLLIYYNNQRP |
| | SGVPDRFSGSRSGTSASLAISGLRSEDEADYYCAAWDDSLSGLVFGTGTKLTVL |
| | (SEQ ID NO: 495) |
| N1wc132 | QSVLTQPPSASGTPGQRVTISCSGSSSNIGSNPVNWYQQLPGSAPKLLVYSDDQR |
| | PSGVPDRFSGSKSGASASLAISGLQSEDESDYYCAAWDDSQNAVVFGGGTKVTV |
| | L (SEQ ID NO: 496) |
| N1wc135 | LPVLTQPPSASGTPGQRVIISCSGSSSNIGSNYVYWYQQLPGTAPKLLIYRNNQRP |
| | SGVPDRFSGSKSGTSASLAISGLRSEDEADYHCAAWDDSLSGVVFGGGTKLTVL |
| | (SEQ ID NO: 497) |
| N1wc150 | SYELTQPPSASGTPGQRVTISCSGSGSNIGKNTVNWYQQLPGAAPKLLIYSNNER |
| | PSGVPGRFSGSKSGTSASLAISGLQSEDETDYYCAAWDDSLNDMVFGGGTKVTV |
| | L (SEQ ID NO: 498) |
| N1wc164 | QAVLTQPSSVSGAPGQRITISCTGTSSNIGAGYDVEWYQHLPGTAPKLLIYRNSNR |
| | PSGVPDRFSASKSGTSASLVITGLQAEDEADYYCQSYDTRLSDSVIFGGGTKLTVL |
| | (SEQ ID NO: 499) |
| N1wc170 | SYELTQPPSVSVAPGQTARITCGGENIGTKSVHWYQQKSGQAPVLVIYEDKKRPS |
| | GIPDRFSGSSSGTMATLTITGAQVDDEADYYCYSADNTGDQRMFGGGTKLTVL |
| | (SEQ ID NO: 500) |
| N1wc177 | QAVLTQPSSASGTPGQRVIISCSGSSSNIGKYIVNWYQQLPGAAPKLLIYTYNERPS |
| | GVPARFSGSTSGTSASLAITGLQSEDEADYYCASWDDSLNVVVFGGGTKLTVL |
| | (SEQ ID NO: 501) |
| N1wc182 | QSVVTQPPSASGTPGQRVTISCSGSSSNIGSNTVNWYQQLPGMAPKLLIYSNNQR |
| | PPGVPDRFSGSKSGTSASLAISGLQSEDEADYYCAAWDDSLNGNWVFGGGTKLT |
| | VL (SEQ ID NO: 502) |
| N1wc197 | QSVLTQPPSASGTPGQRVTISCSGSSSNIGSNYVYWYQKFPGTAPKLLIYTNNQR |
| | PSGVPDRFSGSKSGTSASLAISGLRSEDEADYYCAAWDDSLSGVVFGGGTKVTVL |
| | (SEQ ID NO: 503) |
| N1wc225 | QSVVTQPPSVSGAPGQRVTISCTGSSSNIGAGYDVHWYQQLPGTAPKLLIYGNSN |
| | RPSGVPDRFSGSKSGTSASLAITGLQAEDEADYYCQSYDSSLSGSVFGGGTKLTV |
| | L (SEQ ID NO: 504) |
| N1wc239 | QSVLTQPPSASGTPGQRVAISCSGSHSNIRSNDVYWYQQFPGTAPKLLIYSNDQR |
| | PSGVSDRFAGSKSGTSASLAISGLRSEDESDYYCATWDDSLSGVVFGGGTKLTVL |
| | (SEQ ID NO: 505) |
| N1wc241 | QSVLTQPPSASGTPGQRVTISCSGSFSNIGSNNVYWYQQLAGTAPKLLIYRNNER |
| | PSGVPDRFSGSKSGTSASLAISGLRSEDEADYYCAVRDDSLSGHYVFGSGTKVTV |
| | L (SEQ ID NO: 506) |
| N1wc248 | QSVLTQPPSASGTPGQRVTISCSGSSSNIGSNSVYWYQQFPGTAPKLLIYRNNQR |
| | PSGVPDRFSGSKSDTSASLAISGLRSEDEADYYCATWDDSLSGVVFGGGTKLTVL |
| | (SEQ ID NO: 507) |
| N1wc266 | QAVLTQPSSVSGTPGQRVTMSCSGGRSNIGSNAVNWYQQFPGTAPKLLMYNTN |
| | QRPSGVPGRFSGSKSGASASLAISGLQSDDEADYYCSSWDDSLNGLEFGGGTKL |
| | EIK (SEQ ID NO: 508) |
| N1wc281 | QSVLTQPPSVSVSPGQTASITCSGDKLGDKYTFWYQQKPGQSPVLIIYQDTKRPS |
| | GIPERFSGSNSGNTATLTISGTQAMDEADYYCQAWDSNTDHVIFGGGTKLTVL |
| | (SEQ ID NO: 509) |
| N1wc289 | QAVLTQPSSLSASPGASASLTCTLRSDINLETSRIYWFQQKPGSPPRYLLRYQSDS |
| | DNNLDSGVPSRFSGSKDASANAGILLISGVQSEDEADYHCMIWHSGGSVFGGGTK |
| | VTVL (SEQ ID NO: 510) |
| N1wc295 | QSVLTQPPSASGTPGQRVTISCSGNRSNIGSNTVNWYQQLPGTAPKLLIYSNDQR |
| | PSGVPDRFSGSKSGTSASLTISGLQSDDEADFYCAAWDDNLNGYVFGSGTKVTVL |
| | (SEQ ID NO: 511) |
| N1wc301 | QSVVTQPPSASGTPGQRVTISCSGSSSNIGKNNVYWYQQLPGAAPKLLIYRNHER |
| | ASGVPDRFSGSKSGTSASLAISGLQSEDEADYYCAARDDSLNGYVFGTGTKVTVL |
| | (SEQ ID NO: 512) |
| N1wc319 | QSVLTQPPSSSGTPGQRVTISCSGSSSNIGSNSVNWYQQLPGAAPKLLIYSDNQR |
| | PSGVPDRFSASKSYTSASLAISGLQSGDEADYFCATWDDSLTGLVFGGGTKLTVL |
| | (SEQ ID NO: 513) |
| N1wc336 | QAVLTQPSSASGTPGQRVTISCSGSYSNIGSNYVYWYQQLPGTAPKLLIYTNNQR |
| | PSGVPDRFSGSKSGTSASLVISGLRSDDEGDYYCAAWDDSVSVLLFGGGTKLTVL |
| | (SEQ ID NO: 514) |
| N1wc338 | QSVLTQPPSASGTPGQRVTISCSGSSSNIGTYTVNWYQQHPGTAPKLLIYSNDQR |
| | PSGVPDRFSGSRSGTSASLAISGLRSEDEADYYCAAWDDSLHGYVFRTGTKLTVL |
| | (SEQ ID NO: 515) |
| N1wc403 | QAVLTQPSSASGTPGQRVTISCPGSSSNIGSNTVNWYQQLPGTAPKLLIYTNNQR |
| | PSGVPDRFSASKSGTSASLAISGLQSEDEADYYCAAWDDSLNGYVFGTGTKLTVL |
| | (SEQ ID NO: 516) |
| N1wc441 | SYELTQPPSVSVSPGQTARITCSGDALPDKYVHWYQQKSGQAPVLVLFDDSKRPS |
| | GIPERFSGSSSGTVATLIISGAQVEDEGDFFCYSTDSSGNERVFGGGTKLTVL |
| | (SEQ ID NO: 517) |
| N1wc476 | QAVLTQPSSVSGTPGQTVTISCSGSNSNIGNNYVYWYQQLPGSAPKVVIYRNHQR |
| | PSGVPDRFSGSKSGTAASLAISGLRSEDEADYYCASWDDSLSGLVFGGGTKLTVL |
| | (SEQ ID NO: 518) |
| N1wc516 | QPVLTQPPSASGTPGQRVTISCSGSSSNIGSNTVNWYQQLPGTAPKLLIYSNNQR |
| | PSGVPDRFSGSKSGTSASLAISGLQSEDEADYYCAAWDDSLNDVVFGGGTKLTVL |
| | (SEQ ID NO: 519) |
| N1wc535 | QSVLTQPPSASGTPGQRVTLSCSGSSSNIGSNTVNWYQQLPGTAPKVLIYSDNQR |
| | PSGVPDRFSGSKSGTSASLAISGLQSDDEAVYYCAGWDDSLNAYLFGTGTKVTVL |
| | (SEQ ID NO: 520) |
| N1wc540 | QAVLTQPSSASGTPGQRVTISCSGSSSNIGSYTVNWYQQVPGTAPKLLIYSNNQR |
| | PSGVPERFSGSKSGTSASLAISGLQSEDEADYYCAAWDDSLSDVLFGGGTKLTVL |
| | (SEQ ID NO: 521) |
| N1wc558 | LPVLTQPPSASGTPGQRVTISCSGSSSNIGSNSVNWYQQLPGTAPKLLIYSNNQRP |
| | SGVPDRFSGSQSGTSASLAISGLRSEDEADYYCAAWDDSLNNLVFGGGTKLTVL |
| | (SEQ ID NO: 522) |
| N1wc570 | QSVLTQPPSASATPGQTVTISCSGSTSNIGNNNVNWYQQVPGTAPKLLMYSNNQ |
| | RPSGVPDRFSGSRSGTSASLAITALQSEDEADYYCATWDDSLSAWVFGGGTKVT |
| | VL (SEQ ID NO: 523) |
| N1wc572 | LPVLTQPPSASGTPGQRVTISCSGSSSNIGSNPVNWYQQLPGSAPKLLVYSDDQR |
| | PSGVPDRFSGSKSGASASLAISGLQSEDESDYYCAAWDDSLNAVVFGGGTKVTVL |
| | (SEQ ID NO: 524) |
| N1wc586 | DIVMTQSPSTLSASVGDRVTITCRASQGISSWLAWYQQKPGRAPKVLIYKASTLES |
| | GVPSRFSGSGSGTDFTLTISSLQPEDFATYYCQQSYSTPWTFGQGTKLEIK (SEQ |
| | ID NO: 525) |
| N1wc614 | QSVLTQPPSASGTPGQRVTISCSGSSSNIGSNTVNWYQQLPGTAPKLLIYSNNQR |
| | PSGVPDRFSGSKSGTSASLAISGLQSEDEADYYCAAWDDSLNGLVFGGGTKVTVL |
| | (SEQ ID NO: 526) |
| N1wc621 | QAVLTQPSSVSGAPGQTVTISCAGSSSNLGAGYDAHWYQHLPGAVPRLLIYNNSK |
| | RPSGVPDRFSASKSGSSASLAITGLQPEDEADYYCQTYDTRLSGSMLFGGGTKVT |
| | VL (SEQ ID NO: 527) |
| N1wc624 | QSVLTQPPSASGTPGQGVTISCSGSSSNIGSNTVNWYQQLPGTAPKLLIYSNNQR |
| | PSGVPDRFSGSKSGTSASLAISGLQSEDEADYYCAAWDDSLNGKVFGTGTKVTVL |
| | (SEQ ID NO: 528) |
| N1wc633 | QAVLTQPSSASGTPGQRVTISCSGSSSNIGSYTVNWYQQVPGTAPKLLIYSNNQR |
| | PSGVPERFSGSKSGTSASLAISGLQSEDEADYYCAAWDDSLSDVLFGGGTKVTVL |
| | (SEQ ID NO: 529) |
| N1wc39 | DIQLTQSPSSVSASIGDTVTITCRASQNINKWLAWYQQKPGKAPYLLISTASNLQSG |
| | VPSRFSGTGSGTAFTLTISRVQPEDFANYYCQQAFSFPITFGQGTRLEIK (SEQ ID |
| | NO: 530) |
| N1wc45 | SSELTQDPAVSVALGQTVRITCQGDSLRSYYASWYQQKPGQAPVLVIYGKNNRPS |
| | GIPDRFSGSSSGNTASLTITGAQAEDEADYYCNSRDSSGNHVVFGGGTKLTVL |
| | (SEQ ID NO: 531) |

B. DNA sequence

Heavy Chain
>hN1S3-001
GAGGTGCAGCTGGTGCAGTCTGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
ACTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCAGGGGACAATGGT
CACCGTCTCGAGT (SEQ ID NO: 532)
> hN1S3-003
GAGGTCCAGCTGGTGCAGTCTGGGCCTGAGGTGAAGAAGCCTGGGACCTCAGTGAAGGTCTCCTG
CAAGGCTTCTGGATTCACCTTTACTAACTCTGCTGTGCAGTGGGTGCGACAGGCTCGTGGACAACG
CCTTGAGTGGATAGGATGGATCGTCGTTGGCAGTGGTAACACAAACTACGCACAGAAGTTCCAGGA
AAGAGTCACCATTACCAGGGACATGTCCACAAGCACAGCCTACATGGAGCTGAGCAGCCTGAAATC
CGAGGACACGGCCGTGTATTATTGTGTGGCAGATACGGTCTACTGGGGCCAGGGCACCCTGGTCAC
CGTCTCGAGT (SEQ ID NO: 533)
> hN1S3-007
CAGGTGCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCAGGGGACAATGGT
CACCGTCTCGAGT (SEQ ID NO: 534)
> hN1S3-023
GAGGTGCAGCTGGTGCAGTCTGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
ACTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCAGGGGACAATGGT
CACCGTCTCGAGT (SEQ ID NO: 535)
> hN1S3-032
GAGGTGCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCAAGGGAACCCTGGT
CACCGTCTCGAGT (SEQ ID NO: 536)
>hN1S3-035
CAGGTCCAGCTGGTGCAGTCTGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCAAGGCACCCTGGT
CACCGTCTCGAGA (SEQ ID NO: 537)
> hN1S3-040
GAGGTCCAGCTGGTGCAGTCTGGGCCTGAGGTGAAGAAGCCTGGGACCTCAGTGAAGGTCTCCTG
CAAGGCTTCTGGATTCACCTTTACTAGCTCTGCTGTGCAGTGGGTGCGACAGGCTCGTGGACAACG
CCTTGAGTGGATAGGATGGATCGTCGTTGGCAGTGGTAACACAAACTACGCACAGAAGTTCCAGGA
AAGAGTCACCATTACCAGGGACATGTCCACAAGCACAGCCTACATGGAGCTGAGCAGCCTGAGATC
CGAGGACACGGCCGTGTATTATTGTGTGGCAGATACGGTCTACTGGGGGCGAGGGACCACGGTCA
CCGTCTCGAGT (SEQ ID NO: 538)
> hN1S3-053
GAAGTGCAGCTGGTGCAGTCTGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGGCGGGGGACCACGGT
CACCGTCTCGAGT (SEQ ID NO: 539)
> hN1S3-055
CAGGTCCAGCTGGTGCAGTCTGGAGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
ACCTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCGGGGGACAATGGT
CACCGTCTCGAGT (SEQ ID NO: 540)
> hN1S3-147
CAGGTGCAGCTGGTGCAGTCTGGGCCTGAGGTGAAGAAGCCTGGGACCTCAGTGAAGGTCTCCTG
CAAGGCTTCTGGATTCACCTTTACTAGCTCTGCTGTGCAGTGGGTGCGACAGGCTCGTGGACAACG
CCTTGAGTGGATAGGATGGATCGTCGTTGGCAGTGGTAACACAAACTACGCACAGAAGTTCCAGGA
AAGAGTCACCATTACCAGGGACATGTCCACAAGCACAGCCTACATGGAGCTGAGCAGCCTGAGATC
CGAGGACACGGCCGTGTATTACTGTGTGGCAGATACGGTCTACTGGGGCAAAGGAACCCTGGTCAC
CGTCTCGAGT (SEQ ID NO: 541)
> hN1S3-082
CAGGTGCAGCTGGTGCAATCTGGGCCTGAGGTGAAGAAGCCTGGGACCTCAGTGAAGGTCTCCTG
CAAGGCTTCTGGATTCACCTTTACTAGCTCTGCTGTGCAGTGGGTGCGACAGGCTCGTGGACAACG
CCTTGAGTGGATAGGATGGATCGTCGTTGGCAGTGGTAACACAAACTACGCACAGAAGTTCCAGGA
AAGAGTCACCATTACCAGGGACATGTCCACAAGCACAGCCTACATGGAGCTGAGCAGCCTGAGATC
CGAGGACACGGCCGTGTATTACTGTGTGGCAGATACGGTCTACTGGGGCAGGGGAACCCTGGTCA
CCGTCTCGAGT (SEQ ID NO: 542)
> hN1S3-088
GAGGTGCAGCTGTTGGAGTCTGGGGGAGGCTTGGTACAGCCTGGGGGGTCCCTGAGACTCTCCTG
TGCAGCCTCTGGATTCACCTTTAGCAGCTATGCCATGAGCTGGGTCCGCCAGGCTCCAGGGAAGGG
GCTGGAGTGGGTCTCAGCTATTAGTGGTAGTGGTGGTAGCACATATTACGCAGACTCCGTGAAGGG
CCGGTTCACCATCTCCAGAGACAATTCCAAGAACACGCTGTATCTGCAAATGAACAGCCTGAGAGCC
GAGGACACGGCCGTGTATTACTGTGCGACTGAGCCTCCTTTTGATATCTGGGGCCGAGGGACAATG
GTCACCGTCTCGAGT (SEQ ID NO: 543)
>hN1S3-096
GAGGTGCAGCTGGTGCAGTCTGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
ACTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGGCGAGGGACCACGGT
CACCGTCTCGAGT (SEQ ID NO: 544)
> hN1S3-112
CAGGTGCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCGGGGAACCCTGGT
CACCGTCTCGAGT (SEQ ID NO: 545)
>hN1S3-124
GAAGTGCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCGAGGGACCACGGT
CACCGTCTCGAGT (SEQ ID NO: 546)
> hN1S3-127
GAAGTGCAGCTGGTGCAGTCTGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
ACTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCAGGGAACCCTGGT
CACCGTCTCGAGT (SEQ ID NO: 547)
> hN1S3-136
CAGGTGCAGCTGCAGGAGTCGGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGGCGGGGGACCACGGT
CACCGTCTCGAGT (SEQ ID NO: 548)
> hN1S3-177
GAGGTGCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCGCAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCGGGGAACCCTGGT
CACCGTCTCGAGT (SEQ ID NO: 549)
> hN1S3-181
CAGGTGCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCGGGGCACCCTGGT
CACCGTCTCGAGT (SEQ ID NO: 550)
> hN1S3-182
CAGGTCCAGCTGGTACAGTCTGGAGCTGAGGTGAAGAAGCCTGGGGCCTCAGTGAAGGTCTCTTGC
AAGGCTTCTGGATACAACTTCACCGGCTACTATATGCACTGGGTGCGACAGGCCCCTGGACAAGGG
CTTGAGTGGATGGGATGGATCAACCCTAACAGTGGTGGCACAAACTATGCACAGAAGTTTCAGGGC
AGGGTCACCATGACCAGGGACACGTCCATCAGCACAGCCTACATGGAGCTGAGCAGGCTGAGATCT
GACGACACGGCCGTGTATTACTGTGCGACAGACTCCTTTGACTACTGGGGCCGAGGGACAATGGTC
ACCGTCTCGAGT (SEQ ID NO: 551)
> hN1S3-184
CAGGTCCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGGAGAGGGACCACGGT
CACCGTCTCGAGT (SEQ ID NO: 552)
> hN1S3-191
CAGGTGCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCGGGGAACCCTGGT
CACCGTCTCGAGT (SEQ ID NO: 553)
> hN1S3-002
GGGGTGCAGCTGGTGCAGTCTGGGGCTGAGGTGAAGAAGCCTGGGGCCTCAGTGAAGGTCTCCTG
CAAGGCTTCTGGATACACCTTCACCGACTACTATGTGCACTGGGTGCGACAGGCCCCGGGCCAAGG
GCCTGAGTGGATGGGATGGATCAACCCTAACAATGGAGGCACAAAGTATGCACAGAAGTTCCAGGG
CAGAGTCACCATGACCACAGACACGTCCACGAGCACCGCCTACATGGAGTTGAGGAGCCTGAGATC
TGACGACACGGCCCTATATTACTGTGCGAGAGATCTGTTACCCTATTACGGTGACTACCGGGCAGAC
AAAATTGATGATTTTGATATCTGGGGCAGGGGAACCCTGGTCACCGTCTCGAGT (SEQ ID NO: 554)
> hN1S3-006
CAGGTGCAGCTGGTGCAGTCTGGGGCTGAGGTGAAGAAGCCTGGGGCCTCAGTGAAGGTCTCCTG
CAAGGCTTCTGGATACAACTTCACCGGCTACTATATACACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCATCATGACCAGGGACACGTCCATCAGCACAGCCTACATGGAACTGAGCAGGCTGAGATC
TGACGACACGGCCGTGTATTACTGTGCGACAGACTCCTTTGACTACTGGGGCCGGGGAACCCTGGT
CACCGTCTCGAGT (SEQ ID NO: 555)
> hN1S3-030
CAGGTGCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCAGGGGACAATGGT
CACCGTCTCGAGT (SEQ ID NO: 556)
> hN1S3-036
GAGGTGCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCAGAGGGACAATGGT
CACCGTCTCGAGT (SEQ ID NO: 557)
> hN1S3-038
GAGGTGCAGCTGGTGCAGTCTGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCATCATGACCAGGGACACGTCCATCAGCACAGCCTACATGGAACTGAGCAGGCTGACATC
TGACGACACGGCCGTGTATTACTGTGCGACAGACTCCTTTGACTACTGGGGGCGAGGGACCACGGT
CACCGTCTCGAGA (SEQ ID NO: 558)
> hN1S3-074
GAGGTGCAGCTGGTGCAGTCTGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCGGGGGACAATGGT
CACCGTCTCGAGT (SEQ ID NO: 559)
> hN1S3-077
GAGGTCCAGCTGGTACAGTCTGGGGCTGAGGTGAAGAAGCCTGGGGCCCCAGTGAAGGTTTCCTG
CAAGACATCTGGATACGGCTTCAGTAACTACTATATGCACTGGGTGCGACAGGCCCCTGGACAAGG
GCTTGAGTGGCTGGGAATGATCAACCCCAGTGATGGTTCTCAACACTTCGCACAGAGGTTCCGGGA
CAGAGTCTCCTTTACCAGCGACACGTCCACGAGCGCAGTCTACATGGAGCTGGCCGGCCTGAGATC
TGACGACACGGCCGTGTATTACTGTGCGAGAGAGAAAGCGAATTCGATGTACTTTGACTACTGGGG
CCAGGGAACCCTGGTCACCGTCTCGAGT (SEQ ID NO: 560)
> hN1S3-083
CAGGTACAGCTGCAGCAGTCAGGTCCAGGACTGGTGAAGCCCTCGCAGACCCTCTCACTCACCTGT
GCCATCTCCGGGGACAGTATCTCTAGCAGCAGTGCTACTTGGAACTGGATCAGACAGTCCCCATCG
AGAGGCCTTGAGTGGCTGGGAAGGACATACTACAGGTCCAAGTGGTATAATGATTATGCAGTATCTG
TGAAAAGTCGAATAACCATCGACCCTGACACATCCAAGAACCAGTTCTCCCTGCAGCTGAACTCTGT
GACTCCCGAGGACACGGCTGTGTATTACTGTGCAAGACAGCTGGGTGGTAGGATTGAATTCTGGGG
CCAAGGAACCCTGGTCACCGTCTCGAGT (SEQ ID NO: 561)
> hN1S3-087
CAGGTCCAGCTGGTACAGTCTGGGGCTGAGGTGAAGAAGCCTGGGGCCTCAGTGAGGATTTCCTG
CAAGACATCTGGATTCACCTTTACCAGCTACTTTATCCACTGGGTGCGGCAGGCCCCAGGACAAAG
GCCAGAGTGGATGGGCGTTATCAACCCAAGCCGTGGCAACGCAGACTACGCACCGAGGTTCCGTG
GCAGAGTCACCATGACCCGGGACAAGTCCACGCACACTGTTTACATGGATCTGAAGAGCCTCAGAT
CTGACGACGCGGCCATATATTACTGTGCCAGAGATCGGCTTGGTGGCCTTCTTGATTACTACGACAG
TAGTGATTCTTCGAAGAATTTTGATGCTTTTGATTTCTGGGGCCAGGGAACCCTGGTCACCGTCTCG
AGT (SEQ ID NO: 562)
> hN1S3-091
GAGGTCCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCAAGGAACAATGGT
CACCGTCTCGAGT (SEQ ID NO: 563)
> hN1S3-092
GAGGTGCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCAGGGCACCCTGGT
CACCGTCTCGAGT (SEQ ID NO: 564)
> hN1S3-095
GAAGTGCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACGAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCAAGGAACCCTGGT
CACCGTCTCGAGT (SEQ ID NO: 565)
> hN1S3-107
GAGGTGCAGCTGGTGCAGTCTGGACCTGAAGTGAAGAAGCCTGGGGCCTCAGTGAAGGTCTCCTG
CAAGGCTTCTGGATACACCTTCACCGACTACTTTCTCAATTGGGTGCGACAGGCCCCTGGACAAGG
GCTTGAGTGGATTGGGTGGATCAACCCTAACACTGGTGGCACAAACTATGCACAGAACTTTCAGGG
CAGGGTCACCGTGACCAGGGACGCGTCCATCAGCACGGCCTATATGGAGCTAAGGAGCCTGACAT
CTGCCGACACGGCCATATATTATTGTGCGAGAGGGGCCTATGATTACGTGTGGGGGAGTCATCGTT
ATGTCGCTGATGCTTTCGATATCTGGGGCCAAGGCACCCTGGTCACCGTCTCGAGT (SEQ ID NO: 566)
> hN1S3-133
CAGGTCCAGCTGGTGCAGTCTGGGCCTGAGGTGAAGAAGCCTGGGACCTCAGTGAAGGTCTCCTG
CAAGGCTTCTGGATTCACCTTTACTAGCTCTGCTGTGCAGTGGGTGCGACAGGCTCGTGGACAACG
CCTTGAGTGGATAGGATGGATCGTCGTTGGCAGTGGTAACACAAACTACGCACAGAAGTTCCAGGA
AAGAGTCACCATTACCAGGGACATGTCCACAAGCACAGCCTACATGGAGCTGAGCAGCCTGAGATC
CGAGGACACGGCCGTGTATTACTGTGTGGCAGATACGGTCTACTGGGGCCAAGGCACCCTGGTCAC
CGTCTCGAGT (SEQ ID NO: 567)
> hN1S3-135
GAGGTGCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCAAAGGCACCCTGGT
CACCGTCTCGAGT (SEQ ID NO: 568)
> hN1S3-141
CAGGTCCAGCTGGTGCAGTCTGGGGCTGAGGTGAAGAAGCCTGGGGCCTCAGTGAAGGTCTCCTG
CAAGGCTTCTGGATACAACTTCACCGGCTACTATATACACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCATCATGACCAGGGACACGTCCATCAGCACAGCCTACATGGAACTGAGCAGGCTGAGATC
TGACGACACGGCCGTGTATTACTGTGCGACAGACTCCTTTGACTACTGGGGCAGAGGAACCCTGGT
CACCGTCTCGAGT (SEQ ID NO: 569)
> hN1S3-151
CAGGTGCAGCTGGTGCAATCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCGGGGAACCCTGGT
CACCGTCTCGAGT (SEQ ID NO: 570)
> hN1S3-153
CAGGTGCAGCTGCAGGAGTCGGGGGGAGGCTTGGTACAGCCTGGGGGGTCCCTGAGCCTCTCCTG
TGCAACCTCTGGATTCACCTTTGGCAATTATGTCATGAGGTGGGTCCGCCAGGCTCCAGGGAAGGG
GCTGGAGTGGGTCTCAACTATCACCGAGGGTGGTGGTGATTTTATATACTACGCAGACTCCGTGAAG
GGCCGGTTCGCCATCTCCAGGGACAATTCCAAGAGCACGGTGTATCTGCACATGAATGGCCTGAGA
GTCGAGGACACGGCCGTTTATTACTGTGCGAAATCCGGGGGCGATGATAATAGCAACTATAATTTTG
AGTATTGGGGCCAAGGCACCCTGGTCACCGTCTCGAGT (SEQ ID NO: 571)
> hN1S3-190
CAGGTCCAGCTGGTGCAGTCTGGGGCTGAGGTGAAGAAGCCTGGGGCCTCAGTGAGGATTTCCTG
CAAGACATCTGGATTCACCTTTACCAGCTACTTTATCCACTGGGTGCGGCAGGCCCCAGGACAAAG
GCCAGAGTGGATGGGCGTTATCAACCCAAGCCGTGGCAACGCAGACTACGCACCGAGGTTCCGTG
GCAGAGTCACCATGACCCGGGACAAGTCCACGCACACTGTTTACATGGATCTGAAGAGCCTCAGAT
CTGACGACGCGGCCATATATTACTGTGCCAGAGATCGGCTTGGTGGCCTTCTTGATTACTACGACAG
TAGTGATTCTTCGAAGAATTTTGATGCTTTTGATTTCTGGGGCCAAGGCACCCTGGTCACCGTCTCG
AGT (SEQ ID NO: 572)
> hN1S3-108
CAGGTGCAGCTGGTGCAGTCTGGGGCTGAGGTGAGGAAGCCTGGGGCCTCAGTGAAAGTCTCCTG
CAAGGCTTCTGGATACACATTCACCGACTACTATTTACACTGGGTGAGACAGGCCCCTGGACTGGGT
CTTGAGTGGATGGGATGGATCAACCCTAACACTGGTGGCACAGACTATGGACAGAAGTTTCAGGGC
AGGGTCACCATGACCAGGGACACGTCCATCAACACAGCCTACATGGAGCTGAGCAGCCTGAGATCT
GACGACACGGCCGTGTATTATTGTGCGAGAGGGGCCTATGATTACGTGTGGGGGAGTCATCGTTAT
GTCGCTGATGCTTTCGATATCTGGGGCCAAGGGACCACGGTCACCGTCTCCTCA (SEQ ID NO: 573)
> hN1S3-130
AAGGTCCAGCTGGTGCAGTCTGGGGCTGAGGTGAAGAAGCCTGGGGCCTCAGTGAAGGTCTCCTG
CAAGGCCTCTGGATACACTTTCACCGACTTCTATATACACTGGGTGCGACAGGCCCCTGGACAAGG
GCTTGAGTGGATGGGATGGATCAATTCCAAAAGTGGTGACACACACTATGCACAGAAGTTTCAGGGC
AGGGTCACCTTGACCAGGGACACGTCCACCGACACAGCCTACATGGAGCTGAGCAGGCTGACATCT
GACGACACGGCCGTGTATTACTGTGCGAGAGGGGCCTATGATTACGTTTGGGGGAGTTCTCGTTAT
GTCGCTGATGCTTTCGATATCTGGGGCCGGGGGACAATGGTCACCGTCTCCTCA (SEQ ID NO: 574)
>N1wc11
GAAGTGCAGCTGGTGCAGTCTGGGCCTGAGGTGAAGAAGCCTGGGACCTCAGTGAAGGTCTCCTG
CAAGGCTTCTGGATTCACCTTTACTAACTCTGCTGTGCAGTGGGTGCGACAGGCTCGTGGACAACG
CCTTGAGTGGATAGGATGGATCGTCGTTGGCAGTGGTAACATAAACTACGCACAGAAGTTCCAGGAA
AGAGTCACCATTACCAGGGACATGTCCACAAGCACAGCCTACATGGAGCTGAGCAGCCTGAAATCC
GAGGACACGGCCGTGTATTATTGTGTGGCAGATACGGTCTACTGGGGCAGAGGGACAATGGTCACC
GTCTCGAGT (SEQ ID NO: 575)
>N1wc13
CAGGTGCAGCTGGTGCAGTCTGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGAAACGTCCATCAGCACAGCCTACATGGAGCTGAGCAGGCTGAGATC
TGACGACACGGCCGTGTATTACTGTGCGACAGACTCCTTTGACTACTGGGGCCGGGGGACAATGGT
CACCGTCTCGAGT (SEQ ID NO: 576)
>N1wc15
CAGGTACAGCTGCAGCAGTCAGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCAAGGGACAATGGT
CACCGTCTCGAGT (SEQ ID NO: 577)
>N1wc16
CAGGTACAGCTGCAGCAGTCAGGGGCTGAGGTGAAGACGCCTGGGGCGTCAGTCAAGGTCTCCTG
CAAGGCTTCTGGATACACCTTCACCGACAATTATATACATTGGGTGAGACAGGCCCCTGGACAAGGG
CTTGAGTGGATGGGACGGATCAATGTTAACAGTGGTGCCACAAACTATGCACGGAAATTTCAGGGTA
GGGTCACCATGACCGGGGACACGTCCATCAGCACAGCCTACATGGACCTGAGCAGACTGAAGTCTG
ACGACACGGCCGTGTATTACTGTGCGAGAGATTTCCGCTACTCCGGTGTGGACGTCTGGGGCCAAG
GGACAATGGTCACCGTCTCGAGT (SEQ ID NO: 578)
>N1wc21
CAGGTGCAGCTGGTGGAGTCTGGGGCTGAGGTGAAGAAGCCTGGGGCCTCAGTGAAGGTCTCCTG
CAAGGCTTCTGGATACACGTTCACCGACCACTATATACAGTGGCTGCGACAGGCCCCTGGACAAGG
GCTTGAGTGGATGGGATGGATGAATCCGAAGAGTGGTGGCACAAACTATGCACAGAAGTTTCAGGG
CAGGGTCACCCTGACCAGGGACACGTCCATCAGCACAGCCTACATGGACCTGAGTGGGCTGAGATT
TGACGACACGGCCATATATTTCTGTGCGAGCGACCCGACTATACCACCTTCCCCCAACTATGACTAC
TACTACTACGCTATGGACCTCTGGGGCAAGGGCACCCTGGTCACCGTCTCGAGT (SEQ ID NO: 579)
>N1wc24
GAGGTGCAGCTGGTGGAGTCCGGCCCAGGACTGGTGAAGCCTTCGGAGACCCTGTCCCTCACCTG
CACTGTCTCTGGTGGCTCCATGACTTCTAACTTTTGGAGCTGGATCCGGCAGACCCCAGGGAAGGG
ACTGGAGTGGATTGGCTATGTCCTTTATACTGGGAGCACCAACTCCAATCCCTCCCTCAAGAGTCGA
GTCACCATGTCAGTAGACACGTCCAAGAACCAGGTCTCCCTGACCCTGAGCTCTGTGACCGCCGCA
GACACGGCCATTTACTACTGTGCGAGAGGCAATGGCTGGTACCTCCCCTGGGGCAGGGGGACAAT
GGTCACCGTCTCGAGT (SEQ ID NO: 580)
>N1wc72
GAAGTGCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCAAAGGGACAATGGT
CACCGTCTCGAGT (SEQ ID NO: 581)
>N1wc79
CAGGTACAGCTGCAGCAGTCAGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCAAGGGACAATGGT
CACCGTCTCGAGT (SEQ ID NO: 582)
>N1wc82
CAGGTGCAGCTGGTGCAATCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGCGCGACAGACTCCTTTGACTACTGGGGCAAGGGAACCCTGGT
CACCGTCTCGAGT (SEQ ID NO: 583)
>N1wc102
CAGGTCCAGCTGGTGCAGTCTGGGGCTGAGGTGAAGAAGCCTGGGGCCCCGGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCGCCATGACCAGGGACACGTCCATCAGCACAGCCTACATGGAGCTGAGCAGGCTGAGAT
CTGACGACACGGCCGTGTATTACTGTGCGACAGACTCCTTTGACTACTGGGGCCAAGGGACAATGG
TCACCGTCTCGAGT (SEQ ID NO: 584)
>N1wc113
CAGATGCAGCTGGTGCAATCAGGGGCTGAGGTGAAGAGGCCTGGGGCCTCAGTTAACCTTTCCTGC
AAGGCATCTGGATACTCCTTCAGCAGATACTATATCCACTGGGTGCGACAGGCCCCTGGACAGGGA
CTTGAGTTGATGGGACGGATCAACCCTGGAGGTGGTACCACAACCTACGCACAGAAATTCCAGGGC
AGAGTCACCCTGTCCAGGGACACGTCCACGAACACCATCTTCATGGAACTGAACGGCCTCACATCT
GAAGACACGGCCATGTATTATTGTGCGAGGTCGAACTACGATATTTTGGCTGGTTATGGGCCTGATG
CTTTTGATTTCTGGGGCCAAGGGACAATGGTCACCGTCTCGAGT (SEQ ID NO: 585)
>N1wc117
CAGGTGCAGCTGGTGCAATCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCGGGGAACCCTGGT
CACCGTCTCGAGT (SEQ ID NO: 586)
>N1wc125
CAGGTACAGCTGCAGCAGTCAGGGGCTGAGGTGAAGAAACCTGGGGCCCCACTGAAGGTCTCCTG
CAAGACTTCTGGATTCACCTTCACCGGCTACTATATACACTGGGTGCGACAGGCCCCTGGACAAGG
GCTTGAGTGGATGGGACGGGTCGACCCTTACAATGGTGCCACAAACTTTGCACAGAAGTTTCAGGG
CAGAGTCACCATGACCACAGACACATCCACGAGCACAACCTACATGGAACTGATGAGCCTGAGATCT
GACGACACGGCCGTATATTACTGTGCGAGAGGGTGGGAACTTGACTCCTGGGGCCAAGGAACCCT
GGTCACCGTCTCCTCA (SEQ ID NO: 587)
>N1wc127
CAGGTGCAGCTGGTGCAGTCTGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCGGGGAACCCTGGT
CACCGTCTCGAGT (SEQ ID NO: 588)
>N1wc129
CAGGTGCAGCTGGTGCAGTCTGGGGCTGAGGTGAAGAAGCCTGGGGCCTCAGTGAAGGTCTCCTG
CAAGGCTTCTGGATACAATTTCACCGGCTACTATTTGCACTGGGTGCGACAGGCCCCTGGACAAGG
GCTTGAGTGGATGGGATGGATCGACCCTAACAGTGGTGGCACAGACTATGCACAGCAGTTTCAGGG
CAGGGTCACCGTGACCAGGGACACGTCCATCAGCACAGCCTACATGGAATTGAGCGGGCTGAGATC
TGACGACACGGCCGTGTACTACTGTGCGTCAGACTCTTTTGACCACTGGGGCCAGGGAACCCTGGT
CACCGTCTCGAGT (SEQ ID NO: 589)
>N1wc130
CAGGTACAGCTGCAGCAGTCAGGAGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGAC
CTGACGACACGGCCGTGTATTACTGTGCGACAGACTCCTTTGACTACTGGGGCAAAGGGACAATGG
TCACCGTCTCGAGT (SEQ ID NO: 590)
>N1wc131
GAGGTCCAGCTGGTACAGTCTGGAGCTGAGGCGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
ACTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCAAGGCACCCTGGT
CACCGTCTCGAGT (SEQ ID NO: 591)
>N1wc132
CAGGTCCAGCTGGTACAGTCTGGAGCTGAGGTGAAGAAGCCTGGGGCCTCAGTGAAGGTCTCCTG
CAAGGCTTCTGGATACAATTTCACCGGCTACTATTTGCACTGGGTGCGACAGGCCCCTGGACAAGG
GCTTGAGTGGATGGGATGGATCAACCCTAACAGTGGTGGCACAGACTATGCACAGAAGTTTCAGGG
CAGGGTCACCGTGACCAGGGACACGTCCATCAGCACAGCCTACATGGAACTGGGCGGGCTGAGAT
CTGACGACACGGCCGTGTACTACTGTGTGTCCGACTCTTTTGACCACTGGGGCCGGGGCACCCTGG
TCACCGTCTCGAGT (SEQ ID NO: 592)
>N1wc135
CAGGTCCAGCTGGTGCAGTCTGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCAGGGCACCCTGGT
CACCGTCTCGAGT (SEQ ID NO: 593)
>N1wc150
GAAGTGCAGCTGGTGCAGTCTGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
ACTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTATATGGAGCTGAGCAGGCTGAGATC
TGACGACACGGCCGTGTATTACTGTGCGACAGACTCCTTTGACTACTGGGGCCGGGGGACAATGGT
CACCGTCTCGAGT (SEQ ID NO: 594)
>N1wc164
CAGGTGCAGCTGGTGCAGTCTGGGGCTGACGTGAAGAAGCCTGGGGCCTCAGTGAAAATTTCCTGC
AAGGCATCTGGATACACCTTCACCAAGTACTATATGCACTGGGTGCGACAGGCCCCTGGACAAGGA
CTTGAGTGGATGGGAATAATCAACCCTACTAATGGTTACACAAGCTACGCACAGAAGTTCCAGGGCA
GAGTCACCATGACCAGTGACACGTCCGCGAGCACAGTCTCCATGGAGCTGAGCAGCCTGAGATCTG
AAGACACGGCCGTGTATTACTGTACGAGATCTTACAGTAACTACGATTCTTTTGATATCTGGGGCAG
GGGGACAATGGTCACCGTCTCGAGT (SEQ ID NO: 595)
>N1wc170
CAGGTACAGCTGCAGCAGTCAGGACCTGAGGTGAAGAAGCCTGGGGCCTCAGTGAAAATCTCCTGC
AAGACTTCTGGTTATACCTTTAGCAGTTTCGGCATCAGTTGGGTGCGACAGGCCCCTGGACAAGGG
CCTGAGTGGATGGGATGGGTCAGTCCTTACAGTGGTGACACGGACTATGCACAGAAGTTCCGGGGC
AGAGTCACCATGACCATAGACTCATCCACGGACACAGCCTACGTGGAGATGAGGAGCCTGAGGTCT
GACGACACGGCCATATATTATTGTGCGAATTCACCTATCTCTGATGGCAGTTCGTCCTCGTATGACAA
CTGGGGCAAAGGCACCCTGGTCACCGTCTCGAGT (SEQ ID NO: 596)
>N1wc177
CAGGTGCAGCTGGTGCAGTCTGGGGCTGAGGTGAAGGAGCCTGGGTCCTCGGTGAGGGTCTCCTG
TAAGTCTTCTGGAGGAACCTTCAGCAATTATGCAATCAGCTGGGTGCGACAGGCCCCTGGACAAGG
GCTTGAGTGGATGGGAGGGATCATCCCAGGCTTTGGGAAAACAAATTACGCACAGAAGTTCCAGGG
CAGAGTCACGATCACCGCGGACGCATCCACGAGTACAACCTACATGGAACTGCGCACCTTAAGATC
TGGCGACACGGCCGTATACTACTGTGCGAGAGATCGGGGCCCCGATATAGGAGTGGCTCATTTTGA
CTCCTGGGGCAAGGGGACAATGGTCACCGTCTCGAGT (SEQ ID NO: 597)
>N1wc182
CAGGTCCAGCTGGTGCAGTCTGGGCCTGAGGTGAAGAAGCCTGGGACCTCAGTGAAGGTCTCCTG
CAAGGCTTCTGGATTCACCTTTACTAGCTCTGCTGTGCAGTGGGTGCGACAGGCTCGTGGACAACG
CCTTGAGTGGATAGGATGGATCGTCGTTGGCAGTGGTAACACAAACTACGCACAGAAGTTCCAGGA
AAGAGTCACCATTACCAGGGACATGTCCACAAGCACAGCCTACATGGAGCTGAGCAGCCTGAGATC
CGAGGACACGGCCGTGTATTACTGTGTGGCAGATACGGTCTACTGGGGCCAAGGCACCCTGGTCAC
CGTCTCGAGT (SEQ ID NO: 598)
>N1wc197
GAGGTGCAGCTGGTGCAGTCTGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCGAGGAACCCTGGT
CACCGTCTCGAGT (SEQ ID NO: 599)
>N1wc225
CAGGTACAGCTGCAGCAGTCAGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCGAGGGACAATGGT
CACCGTCTCGAGT (SEQ ID NO: 600)
>N1wc239
GAGGTGCAGCTGGTGGAGTCCGGGGCTGAGGTGAAGAAGCCTGGGGCCTCAGTGAAGGTCTCCTG
CAAGGCTTCTGGATACAACTTCACCGGCTACTATATACACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCATCATGACCAGGGACACGTCCATCAGCACAGCCTACATGGAACTGAGCAGGCTGAGATC
TGACGACACGGCCGTGTATTACTGTGCGACAGACTCCTTTGACTACTGGGGCAAGGGGACAATGGT
CACCGTCTCGAGT (SEQ ID NO: 601)
>N1wc241
GAGGTCCAGCTGGTGCAGTCTGGAGCGGAGGTGAAGAAGCCTGGGGCCTCAGTGAAGGTCTCATG
TAAGACTTCTGGATATACCTTCAACGACTACTACCATCACTGGGTGCGGCAGGCCCCTGGACAAGG
CCTTGAGTGGATGGGATGGATCAACCCGCATACAGGTGTCGCAAACTATGCAGAGAAGTATAAGGG
CAGGCTCGCCATGACCGGGGACACGTCCATCAGTACAATTTACATGGACCTGAGTAGTCTCATTTCT
GACGACACGGCCATATATTTCTGTGCGAGAGAGAGGTCAGAGTGGAGGAACACAGTGTCGTCTCCC
TCTGAATACTTCCACCACTGGGGCAAGGGAACCCTGGTCACCGTCTCGAGT (SEQ ID NO: 602)
>N1wc248
GAGGTGCAGCTGGTGCAGTCTGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACTCGGCCGTGTATTACTGTGCGACAGACTCCTTTGACTACTGGGGCCGAGGAACCCTGGT
CACCGTCTCGAGT (SEQ ID NO: 603)
>N1wc266
CAGGTGCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCAGGGGACAATGGT
CACCGTCTCGAGT (SEQ ID NO: 604)
>N1wc281
CAGGTGCAGCTGCAGGAGTCGGGGGCTGAGGTGAAGAAGCCTGGGGCCTCCGTGAAGGTTTCCTG
CAGGGCATCTGGATACACCTTCACCACTAACTCTCTGCACTGGGTGCGACAGGCCCCTGGACAAGG
ACTTGACTGGATGGGAGTCATCAACCCCAGGGACGGTAGCGCAACCGTCGCACAGAAATTCCAGGG
CAGGATCACCATGACCAGTGACACGTCAACGAGCACAGTCTTCTTGGCGCTGAGCAGCCTGACATC
TGACGACACGGGCGTATATTTCTGTGCGAGAGATGGCCTGGATTACCGTGATACAAGTCGAATCCTT
GCCCCGACTGATGTCTGGGGCAGAGGCACCCTGGTCACCGTCTCGAGT (SEQ ID NO: 605)
>N1wc289
CAGGTCCAGCTGGTACAGTCTGGGGCTGAGGTGAAGAAGCCTGGGACTACAGTTAAAATCTCCTGC
AAGGTTTCTGGATACAAGTTCACCGACTACTACATACACTGGGTACGACAGGCCCCAGGAAAAGGG
CTTGAGTGGATGGGATTTGTTGATCCTACAGATGATGAAACAAGATTCGCAGAGAAGTTCCAGGGCA
GACTCACCATGACCGCGGACACGTCTAGAGACACAGCCTCCATGGAACTGAGCAGCCTGAGATCTG
AGGACACGGCCGTGTATTACTGTATAAGGGGGATCGCCGCATTGGCGGACTGGGGCCGGGGGACA
ATGGTCACCGTCTCGAGT (SEQ ID NO: 606)
>N1wc295
CAGGTACAGCTGCAGCAGTCAGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAACTTTCAGGG
CAGGGTCACCATGACCAGGGACACGTCTATCAGCACAGCCTACATGGAGCTGAGCAGGCTGAGATC
TGACGACACGGCCGTTTATTACTGTGCGACAGACTCCTTTGACTACTGGGGCCGGGGGACAATGGT
CACCGTCTCGAGT (SEQ ID NO: 607)
>N1wc301
CAGGTACAGCTGCAGCAGTCAGGGGCTGAGGTGAAGAAGCCTGGGGCCTCAGTGAAGGTTTCCTG
CAAGGCTTCTGGATACATCTTCGGCACCTATGCCATACATTGGGTGCGCCAGGCCCCCGGACAAAG
CCTTGAGTGGATGGGATGGATCAACGCTGGCACCGGTAACACAAAATATTCACAGAAGTTCCAGGG
CAGAGTCACCATTACCATGGACACATCCGCGACCACAGCCTACATGGAGCTGAGCAGCCTGAGATC
TGGAGATACGGCTGTGTATTACTGCACAAGAGGGGATTATTATGATAGTCGTGGTTATTACAACCGC
TTCGACTACTGGGGCAGGGGAACCCTGGTCACCGTCTCGAGT (SEQ ID NO: 608)
>N1wc319
CAGGTGCAGCTGGTGCAGTCTGGGGCTGAGGTGGAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCGGGGAACCCTGGT
CACCGTCTCGAGT (SEQ ID NO: 609)
>N1wc336
CAGGTGCAGCTGGTGCAGTCTGGGGCTGAGGTGAAGAGGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCAAAGGAACCCTGGT
CACCGTCTCGAGT (SEQ ID NO: 610)
>N1wc338
CAGGTGCAGCTCGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTTAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCAAGGGAACCCTGGT
CACCGTCTCGAGT (SEQ ID NO: 611)
>N1wc403
GAGGTGCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCGGGGAACCCTGGT
CACCGTCTCGAGT (SEQ ID NO: 612)
>N1wc441
CAGGTGCAGCTGGTGCAATCTGGGGCTGAGGTGAAGAAGCCTGGGGCCTCAGTGAAGGTCTCCTG
CAAGGCTCCTGGATACAGTTTCACCGACTACTATTTGCACTGGGTGCGACAGGCCCCTGGAATAGG
ACTTGAGTGGGTGGGATGGATCAACCCTAACAATGGTGGCACAAATTATGCACAGAAGTTTCGGGG
CAGGGTCACGATGACCAGGGACACGGCCATCAACACAGCCTACATGGACATGAGGTGGCTGAGATC
TGACGACACGGCCGTGTATTACTGTGCGACAGATGGGCCCCCGGGTACTGGCAATGCTTTTGATAT
CTGGGGCAGGGGCACCCTGGTCACCGTCTCGAGT (SEQ ID NO: 613)
>N1wc476
GAGGTGCAGCTGGTGCAGTCTGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCAGGGAACCCTGGT
CACCGTCTCGAGT (SEQ ID NO: 614)
>N1wc516
GAGGTGCAGCTGGTGGAGTCTGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCATCATGACCAGGGACACGTCCATCAGCACAGCCTACATGGAACTGAGCAGGCTGACATC
TGACGACACGGCCGTGTATTACTGTGCGACAGACTCCTTTGACTACTGGGGCAGGGGCACCCTGGT
CACCGTCTCGAGT (SEQ ID NO: 615)
>N1wc535
CAGGTCCAGCTGGTACAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGCGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCGAGGAACCCTGGT
CACCGTCTCGAGT (SEQ ID NO: 616)
>N1wc540
GAAGTGCAGCTGGTGCAGTCTGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCAAGGGACAATGGT
CACCGTCTCGAGT (SEQ ID NO: 617)
>N1wc558
CAGGTCCAGCTGGTACAGTCTGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCAGGGGCACCCTGGT
CACCGTCTCGAGT (SEQ ID NO: 618)
>N1wc570
CAGGTCCAGCTGGTGCAGTCTGGGCCTGAGGTGAAGAAGCCTGGGACCTCAGTGAAGGTCTCCTG
CAAGGCTTCTGGATTCACCTTTACTAGCTCTGCTGTGCAGTGGGTGCGACAGGCTCGTGGACAACG
CCTTGAGTGGATAGGATGGATCGTCGTTGGCAGTGGTAACACAAACTACGCACAGAAGTTCCAGGA
AAGAGTCACCATTACCAGGGACATGTCCACAAGCACAGCCTACATGGAGCTGAGCAGCCTGAGATC
CGAGGACACGGCCGTGTATTATTGTGTGGCAGATACGGTCTACTGGGGCAGAGGGACAATGGTCAC
CGTCTCGAGT (SEQ ID NO: 619)
>N1wc572
CAGGTGCAGCTGGTGCAGTCTGGAGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGTTGAGATC
TGACGACACGGCCGTGTATTACTGTGCGACAGACTCCTTTGACTACTGGGGCCGAGGAACCCTGGT
CACCGTCTCGAGT (SEQ ID NO: 620)
>N1wc586
CAGGTCCAGCTGGTACAGTCTGGGCCTGAGGTGAAGAAGCCTGGGGCCTCAGTGAAGGTCTCCTG
TAAGGCAGCTGGATACACCTTTCCCGACTTCTATATGCACTGGGTGCGACAGGCCCCTGGAAAAGG
ACCTGAGTGGGTGGGATGGATCAACTCTAATAGTGGCAGCACAAGACTTGCAGAGAGACTTGAGGG
CAGGGTCACCTTGACCAGGGACACATCCATCAGCACAGCCTATATGGAGTTGACCAGCCTCACATCT
GACGACACGGCCGTCTATTATTGTGCGAGAGGGGCCTATGATTACGTCTGGGGGACTTCTCGCTAT
GTCGCTGATGCTTTCGATATCTGGGGCCAAGGAACTCTGGTCACCGTCTCCCCA (SEQ ID NO: 621)
>N1wc614
GAGGTGCAGCTGGTGCAGTCTGGGGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCAAGGCACCCTGGT
CACCGTCTCGAGT (SEQ ID NO: 622)
>N1wc621
GAGGTGCAGCTGGTGCAGTCTGGGGCTGAGGTGAAGAAGCCTGGGGCCTCTGTGAAGGTCTCCTG
CAAGGCTTCTGGATACACCTTCACCGGCTACTATATACACTGGGTGCGACAGGCCCCTGGTCAAGG
GCTTGAGTGGATGGGCTGGATCAGCCCCAACAGTGGTGTCACAAACTATGCACAGAGGTTTCAGGG
CAGGGTCACCATGTCCAGGGACACGTCCATCACCACAGCCTACATGGAGCTGAACAGGCTGGGATC
TGACGACACGGCCGTGTATTACTGTGCGAGAGAAGGGCGGGAGGACGGTGTCCTCTTTGACTACTG
GGGCCGAGGAACCCTGGTCACCGTCTCGAGT (SEQ ID NO: 623)
>N1wc624
CAGGTACAGCTGCAGCAGTCAGGAGCTGAGGTGAAGAAGCCTGGGGCCTCAGTGAAGGTCTCCTG
CAAGGCTTCTGGATACAACTTCACCGGCTACTATATGCACTGGGTGCGACAGGCCCCTGGACAAGG
ACTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGGGAT
CTGACGACACGGCCGTGTATTACTGTGCGACAGACTCCTTTGACTACTGGGGCCGAGGCACCCTGG
TCACCGTCTCGAGT (SEQ ID NO: 624)
>N1wc633
CAGGTACAGCTGCAGCAGTCAGGGGCTGAGGTGAAGAAGCCTGGGGCCTCAGTGAAGGTCTCTTG
CAAGGCTTCTGGATACAACTTCACCGGCTACTATATGCACTGGGTGCGACAGGCCCCTGGACAAGG
GCTTGAGTGGATGGGATGGATCAACCCTAACAGTGGTGGCACAAACTATGCACAGAAGTTTCAGGG
CAGGGTCACCATGACCAGGGACACGTCCATCAGCACAGCCTACATGGAGCTGAGCAGGCTGAGATC
TGACGACACGGCCGTGTATTACTGTGCGACAGACTCCTTTGACTACTGGGGCCGAGGAACCCTGGT
CACCGTCTCGAGT (SEQ ID NO: 625)
>N1wc39
GAGGTCCAGCTGGTGCAGTCTGGAGCTGAGGTGAAGAAGCCTGGGGCCTCAGTGAAGCTCTCCTG
CAAGACTTCTGGACACACCTTCACTAACTATGCTTTCCATTGGCTGCGCCAGGCCCCCGGACAAAGG
CCTGAGTGGCTGGGATGGATCAACGCTGACAATGGTAACACAAAATATTCACAGAGTTTCCAGGATA
GAGTCACCATTACCAGGGACATATCCGCGAACACAGCCTACATGGAGATGAGCAGACTGAAATCAG
AAGACACGGCTTATTATTACTGTGCGAGAGATCCCTGGAGTGGCGTCCTTGACCACTGGGGCAAAG
GAACCCTGGTCACCGTCTCGAGT (SEQ ID NO: 626)
>N1wc45
GAGGTCCAGCTGGTGCAGTCTGGAGCTGAGGTGAAGAAGCCTGGGGCCTCAGTGAAAGTCTCCTG
TAAGGCTTCTGGTTACAGCTTTCGCAGCTATGGTATCGTCTGGGTGCGACAGGCCCCTGGACAGGG
ACTTGAGTGGATGGGATGGATCAGCGCCTACAATGGTAACACAAACTATGCACAGAAGCTCCAGGG
CAGAGTCACCATGACCACAGACACATCCACGAGCACAGCCTACATGGAGCTGAGGAGCCTGAGATC
TGACGACACGGCCGTGTATTACTGTGTGAGAGGGGACCGCCATTACGATATTCTGACTGGTTACTCC
CCTGTCTGGTTCGACCCCTGGGGGAAGGGGACCACGGTCACCGTCTCGAGT (SEQ ID NO: 627)
Light Chain
> hN1S3-001
CAGTCTGTCGTGACGCAGCCGCCCTCAGCGTCTGGGACCCCCGGGCAGAGGGTCACCATCTCTTG
TTCTGGAAGCAGCTCCAACATCGGAAGTAATACTGTAAACTGGTACCAGCAGCTCCCAGGAACGGC
CCCCAAACTCCTCATCTATAGTAATAATCAGCGGCCCTCAGGGGTCCCTGACCGATTCTCTGGCTCC
AAGTCTGGCACCTCAGCCTCCCTGGCCATCAGTGGGCTCCAGTCTGAGGATGAGGCTGATTATTAC
TGTGCAGCATGGGATGACAGCCTGAATGGTTATGTCTTCGGAACTGGGACCAAGCTGACCGTCCTA
(SEQ ID NO: 628)
> hN1S3-003
CAGTCTGTGCTGACTCAGCCACCCTCAGCGTCTGGGACCCCCGGGCAGAGGGTCACCATCTCTTGT
TCTGGAAGCAGCTCCAACATCGGAAGTAATACTGTAAACTGGTACCAGCAGCTCCCAGGAACGGCC
CCCAAACTCCTCATCTATAGTAATAATCAGCGGCCCTCAGGGGTCCCTGACCGATTCTCTGGCTCCA
AGTCTGGCACCTCAGCCTCCCTGGCCATCAGTGGGCTCCAGTCTGAGGATGAGGCTGATTATTACT
GTGCAGCATGGGATGACAGCCTGAATGGTTGGGTGTTCGGCGGAGGGACCAAGCTGACCGTCCTA
(SEQ ID NO: 629)
> hN1S3-007
CAGGCTGTGCTGACTCAGCCGTCCTCCGTGTCTGGGACCCCCGGCCAGAGGGTCACCATGTCTTGT
TCTGGAGGCAGGTCCAACATCGGAAGTAATGCTGTCAACTGGTACCAACAGTTCCCAGGAACGGCC
CCCAAACTCCTCATGTATAATACTAATCAGCGGCCCTCAGGGGTCCCTGGCCGATTCTCTGGCTCCA
AGTCTGGCGCCTCAGCCTCCCTGGCCATCAGTGGCCTCCAGTCTGATGATGAGGCTGATTATTACT
GTTCGTCTTGGGATGACAGCCTCAATGGTCTGGAGTTCGGCGGAGGGACCAAGCTGACCGTCCTA
(SEQ ID NO: 630)
> hN1S3-023
CAGTCTGTCGTGACGCAGCCGCCCTCAGCGTCTGGGACCCCCGGGCAGAGGGTCACCATCTCTTG
TTCTGGAAGCAGCTCCAACATCGGAAGTAATACTGTAAACTGGTACCAGCAGCTCCCAGGAACGGC
CCTCAAACTCCTCATCTATAGTAATAATCAGCGGCCCTCAGGGGTCCCTGACCGATTCTCTGGCTCC
AAGTCTGGCACCTCAGCCTCCCTGGCCATCAGTGGGCTCCAGTCTGAGGATGAGGCTGATTATTAC
TGTGCAGCATGGGATGACAGCCTGAATGGTTATGTCTTCGGAACTGGGACCAAGCTGACCGTCCTA
(SEQ ID NO: 631)
> hN1S3-032
CAGGCTGTGCTGACTCAGCCGTCCTCAGTGTCTGGGGCCCCAGGGCAGAGGGTCACTATCTCCTGT
ACTGGGAGCAGCTCCAACATCGGGGCAGGTTATGATGTTCACTGGTACCGGCAGCTTCCAGGAACA
GCCCCCAAACTCCTCTTGTATGGTAATGACAATCGACCCTCAGGGGTCCCTGACCGATTCTCTGGCT
CCAAGTCTGGCGCCTCAGCCTCCCTGGCCATCACAGGGCTCCGGGCTGACGATGAGGCTGATTATT
ACTGCCAGTCCTATGACAGCAGCCTGAGTGGTTCGGTGTTCGGCGGAGGGACCAAGGTCACCGTC
CTA (SEQ ID NO: 632)
> hN1S3-035
CAGGCTGTGCTGACTCAGCCGTCCTCAGTGTCTGGGGCCCCAGGGCAGAGGGTCACCATCTCCTG
CACTGGGAGCAGCTCCAACATCGGGGCAGGTTATGATGTACACTGGTACCAGCAGCTTCCAGGAAC
AGCCCCCAAACTCCTCATCTATGGTAACAGCAATCGGCCCTCAGGGGTCCCTGACCGATTCTCTGG
CTCCAAGTCTGGCACCTCAGCCTCCCTGGCCATCACTGGGCTCCAGGCTGAGGATGAGGCTGATTA
TTACTGCCAGTCCTATGACAGCAGCCTGAGTGGTTCGGTATTCGGCGGAGGGACCAAGGTCACCGT
CCTA (SEQ ID NO: 633)
> hN1S3-040
CAGTCTGTGCTGACGCAGCCGCCCTCAGCGTCTGGGACCCCCGGGCAGGGGGTCACCATCTCTTG
TTCTGGAAGCAGCTCCAACATCGGAAGGAATTCTGTAAACTGGTACCAACAACTCCCAGGAACGGCC
CCCAAACTCCTCATCTATGGTGATAATCAGCGGCCCTCAGGGGTCCCTGACCGATTCTCTGGCTCCA
AGCCTGGCACCTCGGCCTCCCTGGCCATCAGTGGGCTCCAGTCTGACGATGAGGCTCATTATTACT
GTGCAGCATGGGATGACAGCCTGAATGGTTGGGTGTTCGGCGGAGGGACCAAGGTCACCGTCCTA
(SEQ ID NO: 634)
> hN1S3-053
CAGGCTGTGCTGACTCAGCCGTCCTCAGTGTCTGGGGCCCCAGGGCAGAGGGTCACCATCTCCTG
CACTGGGAGCAGGTCCAACATCGGGGCAGGTTATGATGTACACTGGTACCAGCAGCTTCCAGGAAC
AGCCCCCAAACTCCTCATCTATGGTAACAGCAATCGGCCCTCAGGGGTCCCTGACCGATTCTCTGG
CTCCAAGTCTGGCACCTCAGCCTCCCTGGCCATCACTGGGCTCCAGGCTGAGGATGAGGCTGATTA
TTACTGCCAGTCCTATGACAGCAGCCTGAGTGGTTCGGTGTTCGGCGGAGGGACCAAGCTGACCGT
CCTA (SEQ ID NO: 635)
> hN1S3-055
CAGTCTGTGCTGACTCAGCCACCCTCCGCGTCTGGGACCCCCGGCCAGAGGGTCACCATCTCTTGT
TCTGGAAGCAGCTCCAACATCGGAAGTAATACTGTAACCTGGTACCAGCAGGTCCCAGGAACGGCC
CCCAAAGTCCTCATCTATACTAATATTCAGCGGCCCTCAGGGGTCCCTGACCGATTCTCTGGCTCCA
AGTCTGGCACCTCAGCCTCCCTGGCCATCAGTGGGCTCCAGTCTGAGGATGAGAGTGATTATTTCT
GTGCAGCATGGGATGACAGCCTGAATGGTGTGGTTTTTGGCGGAGGGACCAAGCTGACCGTCCTA
(SEQ ID NO: 636)
> hN1S3-147
TCCTATGAGCTGACTCAGCCACCCTCAGCGTCTGGGACCCCCGGGCAGAGGGTCACCATCTCTTGT
TCTGGAAGCAGCTCCAACATCGGCAGTAATCCTGTAAACTGGTACCAGCACCTCCCAGGAACGGCC
CCCAAACTCCTCATCTATAGTGATAGTCAGCGGCCCTCAGGGGTCCCTGACCGATTCTCTGGCTCCA
AGTCTGGCACCTCAGCCTCCCTGGCCATCAGTGGGCTCCAGTCTGAGGATGAGGCTGATTATTACT
GTGCAACATGGGATGACAGCCTGAGTGGTTGGGTGTTCGGCGGAGGGACCAAGCTGACCGTCCTA
(SEQ ID NO: 637)
> hN1S3-082
CACGTTATACTGACTCAACCGCCCTCAGCGTCTGGGACCCCCGGGCAGAGGGTCACCATCTCTTGT
TCTGGAGGCGGCTCCAACATCGGAAGTAATACTGTAAACTGGTACCAGCACCTCCCAGGAACGGCC
CCCAAACTCCTCATCTATAATAATGATCAGCGGCCCTCAGGGGTCCCTGACCGATTCTCTGGCTCCA
AGTCTGGCACCTCAGCCTCCCTGGCCATCAGTGGGCTCCAGTCTGAGGATGAGGCTGATTATTACT
GTGCAGCATGGGATGACAGCCTGAATGGTTGGCTGTTCGGCGGAGGGACCAAGCTGACCGTCCTA
(SEQ ID NO: 638)
> hN1S3-088
CAGTCTGTGCTGACTCAGCCACCCTCAGTGTCAGGGACCCCCGGGCAGAGGGTCACCATCTCTTGT
TCTGGAAGCAGCTCCAACATCGGAAATGATGTTGTAAACTGGTACCAACAACTCCCAGGAACGGCCC
CCAAACTCCTCATCTATAATGATTATCAGCGGCCCTCAGGGGTCTCTGACCGATTCTCCGGCTCCAA
GTCTGGCACCTCAGCCTACCTGGCCATCAGTGGGCTCCAGTCTGAAGATGAGGCTGATTATCACTG
TGCAGTATGGGATGACAGCCTGGATGGTTGGGTGTTCGGCGGAGGGACCAAGCTGACCGTCCTA
(SEQ ID NO: 639)
> hN1S3-096
CAGCCTGTGCTGACTCAGCCCCCCTCAGCGTCTGGGACCCCCGGGCAGAGGGTCACCATCTCTTGT
TCTGGAAGCAGCTCCAACATCGCAAGTAATTCTGTATACTGGTACCAGCAGCTCCCAGGAACGGCC
CCCAAACTCCTCATCTATAGGAATAATCAGCGGCCCTCAGGGGTCCCTGACCGATTCTCTGGCTCCC
AGTCTGGCGCCTCAGCCTCCCTGGCCATCAGTGGGCTCCGGTCCGAGGATGAGGCTGATTATTACT
GTGCAGCATGGGATGACAACCTCTATGGTGTGGTATTCGGCGGAGGGACCAAGCTGACCGTCCTA
(SEQ ID NO: 640)
> hN1S3-112
CTGCCTGTGCTGACTCAGCCCCCCTCAGCGTCTGGGACCCCCGGGCAGAGGGTCACCATCTCTTGT
TCTGGAAGTAGCTCCAACATCGGAAATAATTATGTATATTGGTATCAACAACTCCCAGGAACGGCCC
CCAAACTCCTCATCTACAGGAATAATCAGCGGCCCTCAGGGGTCCCTGACCGATTCTCTGCCTCCAA
GTCTGGCACCTCAGCCTCCCTGGTCATCAGTGGCATCCGGTCCGAGGATGAGGCTGAATATTACTG
TGGAGGATGGGATGACAGCCTGAGTGGTCAGGTGGTTTTCGGCGGAGGGACCAAGCTGACCGTCC
TA (SEQ ID NO: 641)
> hN1S3-124
CAGGCTGTGCTGACTCAGCCGTCCTCAGCGTCTGGGACCCCCGGGCAGAGGGTCACCATCTCTTGT
TCTGGAAGCAGCTCCAACATCGGAAGTAATACTGTAAACTGGTACCAGCAGCTCCCAGGAACGGCC
CCCAAACTCCTCATCTATGCTACTAATCAGCGGCCCTCAGGGGTCCCTGACCGATTCTCTGGCTCCG
AGTCTGGCACCTCAGCCTCCCTGGCCATCAGGGGGCTCCAGTCTGAGGATGAGGCTGATTATTACT
GTGCAGCATGGGATGACAGCCTGAATGGGAAAGTATTCGGCGGAGGGACCAAGGTCACCGTCCTA
(SEQ ID NO: 642)
> hN1S3-127
CAGTCTGTGTTGACGCAGCCGCCCTCAGTGTCTGGGGCCCCCGGGCAGAGGGTCACCATCTCTTGT
TCTGGAAGCACCTCCAACATCGGAGGCAATCCTGTGACCTGGTACCAGCAATTCCCAGGAACGGCC
CCCAAACTCCTCGTCTATCGTGATAATCAGCGGCCCTCCGGGGTCGCTGACCGATTCTCTGGCTCC
AGGTCTGGCACCTCAGCCTCCCTGGCCATCAGTGGCCTCCAGTCTGAGGATGAGGCTGATTTTTAC
TGTGCATCATGGGATGACAGCCTGGGTGGTGTGGTGTTCGGCGGTGGGACCAAGCTGACCGTCCTA
(SEQ ID NO: 643)
> hN1S3-136
CAGGCTGTGCTGACTCAGCCGTCCTCAGAGTCTGGGACCCCCGGGCAGAGGGTCACCATCTCTTGT
TCTGGGACCACCTCCAACATCGGAGATAATACTGTCACCTGGTACCAGCAGCTCCCAGGAACGGCC
CCCAAACTCCTCATCTATAGTAACACTCAGCGGCCCTCAGGGGTCCCTGCCCGGTTCTCTGGCTCC
AAGTCTGGCACCTCAGCCTCCCTGGCCATCAGTGGACTCCAGTCTGGGGATGAGGCTGATTATTAC
TGTGCGGCGTGGGATGACAGTCGGAATGGTTATGTCTTCGGAACTGGGACCAAGCTGACCGTCCTA
(SEQ ID NO: 644)
> hN1S3-177
CAGGCTGTGCTGACTCAGCCGTCCTCAGCGTCTGGGACCCCCGGGCAGAGGGTCACCATCTCTTGT
TCTGGAAGCAGCTCCAACATCGGAACTAATTATGTCTACTGGTACCAGCATCTCCCAGGAATGGCCC
CCAAACTCCTCCTCTATAGTAATAATCAGCGGCCCTCAGGGGTCCCTGACCGATTCTCTGGCTCCAA
GTCTGGCCCCTCAGCCTCCCTGGCCATCAGTGGGCTCCGGTCCGAGGATGAGGCTGATTATTACTG
TGCTGCTTGGGATGACAGCCTGAGTGGTCTGGTGTTCGGCGGAGGGACCAAGCTGACCGTCCTA
(SEQ ID NO: 645)
> hN1S3-181
TCCTATGTGCTGACTCAGCCACCCTCAGCGTCTGGGACCCCCGGGCAGAGGGTCACCATCTCTTGT
TCTGGGAGCAGTCCCAACATCGGAAGTAATACTGTCAACTGGTACCAGCAACTCCCAGGAACGGCC
CCCAAACTTCTCATCTATACTAATAATCAGCGGCCCTCAGGGGTCCCTGACCGATTCTCTGGCTCCA
AGTCTGCTACCTCAGCCTCCCTGGCCATCAGTGGGCTCCAGTTTGACGATGAGGCTGATTATTTCTG
TGCAGCGTGGGACGACAGCCTTCTTGGTGTGGTCTTCGGCGGAGGGACCAAGGTCACCGTCCTA
(SEQ ID NO: 646)
> hN1S3-182
CAGGCTGTGCTGACTCAGCCGTCCTCAGTGTCCGGGGCCCCAGGGCAGAGGGTCACCATCTCCTG
CACTGGGAGCAGCTCCAACATCGGGTCACCTTATGATGTAAACTGGTACCAGCAGTTTCCAGGAACA
GCCCCCAAACTCCTCATCTATGGTAACATTAATCGGCCCTCAGGAGTCCCTGATCGATTCTCTGGCT
CCAAGTCTGGCACCTCAGCCTCCCTGGCCATCACTGGGCTCCAGGCTGAGGACGAGGCTGATTATT
ACTGCCAGACCTATGACAGCACCCTGAACGGTGCGGTTTTCGGCGGCGGGACCAAGCTGACCGTC
CTA (SEQ ID NO: 647)
> hN1S3-184
CAGTCTGTGCTGACTCAGCCACCCTCAGCGTCTGGGACCCCCGGGCAGAGGGTCACCATCTCTTGT
TCTGGAAGCAGCTCCAACATCGGAGGTAATTATGTATACTGGTACCAGCAGCTCCCAGGAACGGCC
CCCAAACTCCTCATCTATAGGAGTAATCAGCGGCCCTCAGGGGTCCCTGACCGATTCTCTGGCTCCA
AGTCTGGCACCTCAGCCTCCCTGGCCATCAGTGGGCTCCGGTCCGAGGATGAGGCTGATTATTACT
GTGCAGCATGGGATGACAGCCTGCACGGTGTGATATTCGGCGGAGGGACCAAGCTGACCGTCCTA
(SEQ ID NO: 648)
> hN1S3-191
CAGGCTGTGCTGACTCAGCCGTCCTCAGCGTCTGGGACCCCCGGGCAGAGGGTCACCATCTCTTGT
CCTGGAAGCAGCTCCAACATCGGAAGTAATACTGTAAACTGGTACCAGCAGCTCCCAGGAACGGCC
CCCAAACTCCTCATCTATACTAATAATCAGCGGCCCTCAGGGGTCCCTGACCGATTCTCTGCCTCCA
AGTCTGGCACCTCAGCCTCCCTGGCCATCAGTGGGCTCCAGTCTGAGGATGAGGCTGATTATTACT
GTGCAGCATGGGATGACAGCCTGAATGGTTATGTCTTCGGAACTGGGACCAAGCTGACCGTCCTA
(SEQ ID NO: 649)
> hN1S3-002
CAGTCTGTGCTGACTCAGCCACCCTCAGCGTCTGGGACCCCCGGGCAGAGGGTCACCATCTCTTGT
TCTGGAAGCAGCTCCAACATCGGGAGTAACACTGTAAACTGGTACCAGCGACTCCCAGGAGCGGCC
CCCCAACTCCTCATCTACAATAATGACCAGCGGCCCTCAGGGATCCCTGACCGATTCTCTGGCTCCA
AGTCTGGCACCTCGGGCTCCCTGGTCATCAGTGGGCTCCAGTCTGAAGATGAGGCTGATTACTACT
GTGCGTCATGGGATGACAGTCTGAATGGTCGGGTGTTCGGCGGAGGGACCAAGCTGACCGTCCTA
(SEQ ID NO: 650)
>hN1S3-006
CAGGCTGTGCTGACTCAGCCGTCCTCAGCGTCTGGGACCGCCGGGCAGAGGGTCACCATCTCTTG
TTCTGGAAGCAACTCCAACATCGGAAGTAATACTGTAAACTGGTACCAACAACTCCCAGGAACGACC
CCCAAACTCCTCATCTATAGTAATAATGAGCGGCCCTCAGGGGTCCCTGACCGATTCTCTGGCTCCA
AGTCTGGCACTTCAGCCTCCCTGGCCATCAGTGGGCTCCAGTCTGACGATGAAGCTGATTATTACTG
TGCAGCATGGGATGACAGCCTGAATGGTTATGTCTTCGGAACTGGGACCAAGGTCACCGTCCTA
(SEQ ID NO: 651)
> hN1S3-030
CAGTCTGTGTTGACGCAGCCGCCCTCAGCGTCTGGGACCCCCGGGCAGAGGGTCAGCATCTCTTG
TTCTGGAAGCAGCTCCAACATCGGAAGCAATTATGTTTACTGGTACCAGCAACTCCCAGGAACGGCC
CCCAAAGTCCTCGTCTATAGGAATAATCAGCGACCCTCAGGGGTCTCTGACCGCTTCTCTGGCTCCA
AGTATGGCACCTCAGCCTCCCTGGCCATCAGTGGGCTCCGGTCCGAGGATGAGGCTGATTATTACT
GTGGATCATGGGATGACAGCCTGAGTGGTGTGGTATTCGGCGGAGGGACCAAGGTCACCGTCCTA
(SEQ ID NO: 652)
> hN1S3-036
CAGTCTGTGCTGACTCAGCCACCCTCAGCGTCTGGGACCCCCGGGCAGAGGGTCACCATCTCTTGT
TCTGGAAGCAGCTCCAATATCGGAAGTAATTATGTATACTGGTACCAGCAGCTCCCAGGAACGGCCC
CCAAACTCCTCATCTATAGGAATAATCAGCGGCCCTCAGGGGTCCCTGACCGATTCTCTGGCTCCAA
GTCTGGCACCTCAGCCTCCCTGGCCATCAGTGGGCTCCGGTCCGAGGATGAGGCTGATTATTACTG
TGCAGCATGGGATGACAGCCTGAGTGGCGTGGTATTCGGCGGAGGGACCAAGGTCACCGTCCTA
(SEQ ID NO: 653)
> hN1S3-038
CAGTCTGTCGTGACGCAGCCGCCCTCAGCGTCTGGGACCCCCGGGCAGAGGGTCACCATCTCTTG
TTCTGGAAGCAGCTCCAACATCGGAAGTAATTATGTATACTGGTACCAGCAGCTCCCAGGAACGGCC
CCCAAACTCCTCATCTATAGGAATAATCAGCGGCCCTCAGGGGTCCCTGACCGATTCTCTGGCTCCA
AGTCTGGCACCTCAGCCTCCCTGGCCATCAGTGGGCTCCGGTCCGAGGATGAGGCTGATTATTACT
GTGCAGCATGGGATGACAGCCTGAGTGGTGTGGTATTCGGCGGAGGGACCAAGCTGACCGTCCTA
(SEQ ID NO: 654)
> hN1S3-074
CAGTCTGTGCTGACGCAGCCGCCCTCAGCGTCTGGGACCCCCGGGCAGAGGGTCACCATCTCTTG
TTCTGGAGGCAGTTCCAACATCGGAAGTAATACTGTAACCTGGTACCAACAACTCCCAGGAAAGGCC
CCCAAACTCCTCATTTATATCAGTGATCAGCGGCCCTCTGGGGTCCCTGACCGATTCTCTGGCTCCA
AGTCTGGCACCTCAGCCTCCCTGGCCATCAGAGGGCTCCAGTCTGAGGATGAGGCTGATTATTACT
GTGCAGCATGGGATGACAGCCTCAATGGTGCCGTCTTCGGAACTGGGACCAAGGTCACCGTCCTA
(SEQ ID NO: 655)
> hN1S3-077
CAGTCTGTCGTGACGCAGCCGCCCTCACTGTCTGCGGCCCCAGGACAAAGGGTCACCATCTCCTGC
TCTGGAAGCCACTCCAACATTGGTAACTTTTATGTGTCGTGGTACCAGCAAGTCCCAGGAGCAGCCC
CCAAACTCCTCATTTATGACAATAATGAGCGACCCTCAGGGATTCCTGACCGATTCTCTGGCTCCAA
GTCTGGCACGTCTGCCACCCTAGACATCACCGGACTCCAGACTGGGGACGAGGCCGATTATTACTG
CTCAACCTGGGATCGCAGCCTCAGTGCTTCTGTCTTCGGAAGTGGGACCAAGCTGACCGTCCTA
(SEQ ID NO: 656)
> hN1S3-083
CAGTCTGTGCTGACTCAGCCACCCTCCGCGTCCGGGTCTCCTGGACGGTCAGTCACCATCTCCTGC
ACTGGAACCAGCAGGGACGTTGGTGCTTATAACTATGTCTCCTGGTACCAACAACACCCAGGCGAA
GCCCCCAAACTCCTGATTTCTGAGGTCACTAAGCGGCCCTCAGGGGTCCCTGCTCGCTTCTCTGCC
TCCAAGTCTGGCAACACGGCCTCCCTGACCATCTCTGGACTCCAGGCTGAAGATGAGGGTGATTATT
ACTGCAGCTCATATGCAGGCAGCGACAGTTGGGTGTTCGGCGGAGGGACCAAGCTGACCGTCCTA
(SEQ ID NO: 657)
> hN1S3-087
AATTTTATGCTGACTCAGCCCCACTCTGTGTCGGAGTCTCCGGGGAAGACGGTAACCATCTCCTGCA
CCCGCAGCAGTGGCAGCATTGGCAGCAGCTACGTGCAGTGGTACCAGCAGCGCCCGGGCACTTCC
CCTGTCACCATCATCTATGAGGATAGCCAACGGCCCTCTGGGGTCCCTGATCGATTCTCTGGGTCCA
TCGACAACTCCGCCAATTCTGCCTCCCTCACCATCTCTGGACTGAAGACTGAGGACGAGGCTGACTA
CTACTGTCAGTCTTATGATGCCAACAATCGGGTGTTCGGCGGAGGGACCAAGCTGACCGTCCTA
(SEQ ID NO: 658)
> hN1S3-091
CAGTCTGTGCTGACTCAGCCACCCTCAGCGACTGGGACCCCCGGGCAGACAGTCAGCATCTCTTGC
TCTGGAGGCAACTCCAACATCGGAAGTAATTATGTTTCCTGGTATCAGCAACTCCCAGGAACGGCCC
CCAAACTCCTCATCTATAGTAATAATCAGCGGCCCTCAGGGGTCCCTGACCGATTCTCTGGCTCCAA
GTCTGGCACCTCAGCCTCCCTGGCCATCAGTGGGCTCCAGTCTGAGGATGAGGCTGATTATTACTG
TGCAGCATGGGATGACAGCCTGAATGGGCATATCTTCGGAACTGGGACCAAGCTGACCGTCCTA
(SEQ ID NO: 659)
> hN1S3-092
CAGTCTGTGTTGACGCAGCCGCCCTCAGCGTCCGGGACCCCCGGGCAGAGGGTCACCATCTCTTG
TTCTGGAAGCAGCTCCAACATCGGAGCTAATTATGTATACTGGTACCAGCAGCTCCCAGGAACGGCC
CCCAAACTCCTCATTTATAAAAATGATCAGCGGCCCTCAGGGGTCCCTGACCGATTCTCTGGCTCCA
AGTCTGGCACCTCAGCCTCCCTGGCCATCAGTGGGCTCCGGTCCGAGGATGAGGCTGATTATTACT
GTGCAGCATGGGATGACAGCCTGAGTGGTCTGGTATTCGGCGGAGGGACCAAGGTCACCGTCCTA
(SEQ ID NO: 660)
> hN1S3-095
CAGTCTGTGTTGACGCAGCCGCCCTCAGCGTCTGGGACCCCCGGACAGAGGGTCACCATCTCTTGT
TCTGGAAGCAACTCCAACATCGGAAGCAATACTGTGAACTGGTACCAACAGCTCCCAGGAACGGCC
CCCAAAGTCCTCATCTATGGTAATCTTCAGCGGCCCTCAGGGGTCCCTGACCGATTCTCTGGCTCCA
AGTCTGGCACCTCAGCCTCCCTGGCCATCAGTGGGCTCCAGTCTGAGGATGAGGCTGATTATTACT
GTGCAACTTGGGATGACAGCCTGAATGGTTTTGTCTTCGGAACTGGGACCAAGCTGACCGTCCTA
(SEQ ID NO: 661)
> hN1S3-107
CAGTCTGTGCTGACTCAGCCACCCTCAGCGTCTGGGACCCCCGGGCAGAGGGTCACCATCTCTTGT
TCTGGAAGCAGCTCCAACATCGGGAGTAACACTGTAAACTGGTACCAGCGACTCCCAGGAGCGGCC
CCCCAACTCCTCATCTACAATAATGACCAGCGGCCCTCAGGGATCCCTGACCGATTCTCTGGCTCCA
AGTCTGGCACCTCAGGCTCCCTGGTCATCAGTGGGCTCCAGTCTGAAGATGAGGCTGATTACTACT
GTGCGTCATGGGATGACAGTCTGAATGGTCGGGTGTTCGGCGGAGGGACCAAGCTGACCGTCCTA
(SEQ ID NO: 662)
> hN1S3-133
CAGTCTGTGTTGACGCAGCCGCCCTCGGTGTCTGAAGCCCCCAGGCAGAGGGTCACCATCTCCTGT
TCTGGAAGCAGCTCCAACATCGGAAATAATGCTGTAAACTGGTACCAGCAGCTCCCAGGAGAGGCT
CCCAAACTCCTCATCTATTATGATGATCTGCTGCCCTCAGGGGTCTCTGACCGATTCTCTGGCTCCA
AGTCTGGCAGTTCAGCCTCCCTGGCCATCAGTGGGCTCCAGGCTGAGGATGAGGCTGATTATTACT
GTGCTACATGGGATGACAGCCTGAATGCTTGGGTGTTCGGCGGAGGGACCAAGGTCACCGTCCTA
(SEQ ID NO: 663)
> hN1S3-135
CTGCCTGTGCTGACTCAGCCACCCTCGGTGTCTGAAGCCCCCAGGCAGAGGGTCACCATCTCCTGT
TCTGGAAGCAGCTTCAACATCGGAAATAATGCTGTAAACTGGTACCAGCAGCTCCCAGGAAAGGCTC
CCAAACTCCTCATCTATTATGATGATCTGCTGCCCTCAGGGGTCTCTGACCGATTCTCTGGCTCCAA
GTCTGGCACCTCAGCCTCCCTGGCCATCAGTGGGCTCCAGTCTGAGGATGAGGCTGATTATTACTG
TGCAGCATGGGATGACAGCCTGAATGCCGTGGTATTCGGCGGAGGGACCAAGGTCACCGTCCTA
(SEQ ID NO: 664)
> hN1S3-141
CAGTCTGTGCTGACTCAGCCACCCTCAGCGTCTGGGACCCCCGGGCAGAGGGTCACCATCTCTTGT
TCTGGAAGCTACTCCAACATCGGAAGTAATTATGTCTACTGGTACCAGCAGTTCCCAGGAACGGCCC
CCAAACTCCTCATCTATACGAATAATCAGCGGCCCTCAGGGGTCCCTGACCGATTCTCTGGCTCCAA
GTCTGGCTCCTCAGCCTCCCTTGCCATCAGTGGGCTCCGGTCCGAGGATGAGGCTGATTATTACTG
TGCAGCATGGGATGACAGTGTGGGTGTCCTGTTATTCGGCGGGGGGACCAAGCTGACCGTCCTA
(SEQ ID NO: 665)
> hN1S3-151
CAGTCTGTCGTGACGCAGCCGCCCTCAGCGTCTGGGACCCCCGGGCAGAGGGTCACCATCTCTTG
TTCTGGAAGCAGCTCCAACATCGGAAGTAATACTGTAAACTGGTACCAGCAGCTCCCAGGAACGGC
CCCCAAACTCCTCATCTATAGTAATAATCAGCGGCCCTCAGGGGTCCCTGACCGATTCTCTGGCTCC
AAGTCTGGCACCTCAGCCTCCCTGGCCATCAGTGGGCTCCAGTCTGAGGATGAGGCTGATTATTAC
TGTGCAGCATGGGATGACAGCCTGAATGGTGGGGTGTTCGGCGGAGGGACCAAGCTGACCGTCCTA
(SEQ ID NO: 666)
> hN1S3-153
CAGGCTGTGCTGACTCAGCCGTCCTCAGTGTCTGGGGCCCCCGGCCAGAGGGTCATCATCTCCTG
CACTGGAACCAGCTCCAACACCGGGGCAGGCTATTATGTTAATTGGTATCAACAACTTCCGGGAGCA
GCCCCCAAAGTCCTCATCTTTGGTAACGACAATCGGCCCTCAGGGGTCCCTGACCGGTTCTCTGGC
GCCAAGTCTGGCACCTCAGCCTCCCTGGCCATCACTGGGCTCCAGGCTGAAGATGAGGCTGACTAT
TATTGCCAGTCGTACGACCGCAGCCTGAGTGGTTGGGTCTTCGGCGGAGGGACCAAGCTGACCGT
CCTA (SEQ ID NO: 667)
> hN1S3-190
TCTTCTGAGCTGACTCAGGACCCTGCTGTGTCTGTGGCCTTGGGACAGACAGTCAGAATCACATGC
CAAGGAGACAGCCTCAGAAGCAATTATCCAAGCTGGTACCAGCAGAAGCCAGGACAGGCCCCTCTA
CTTGTCATCTATAGTGAAAACAGGCGGCCCTCAGGGATCCCGGACCGATTCTCTGCCTCCAGGTCA
GGAAACACAGCTTCCTTGACCATCACTGGGGCTCAGGCGGAAGATGAGGCTGACTATTACTGTCAC
TCCCGGGGCAGCAGTGGTAACCATAAAGTGTTCGGCGGAGGGACCCAGCTCACCGTTTTA
(SEQ ID NO: 668)
> hN1S3-108
GACATCGTGATGACCCAGTCTCCTTCCACCCTGTCTGCATCTGTAGGAGACAGAGTCACCATCACTT
GCCGGGCCAGTCAGGGTATTAGTAGCTGGTTGGCCTGGTATCAGCAGAAACCAGGGAGAGCCCCT
AAGGTCTTGATCTATAAGGCATCTACTTTAGAAAGTGGGGTCCCATCAAGGTTCAGCGGCAGTGGAT
CTGGGACAGATTTCACTCTCACCATCAGCAGTCTGCAACCTGAAGATTTTGCAACTTACTACTGTCAA
CAGAGTTACAGTACCCCGTGGACGTTCGGCCAAGGGACCAAGCTGGAGATCAAA (SEQ ID NO: 669)
> hN1S3-130
GACATCGTGATGACCCAGTCTCCTTCCACCCTGTCTGCATCTGTAGGAGACAGAGTCACCATCACTT
GCCGGGCCAGTCAGGGTATTAGTAGCTGGTTGGCCTGGTATCAGCAGAAACCAGGGAGAGCCCCT
AAGGTCTTGATCTATAAGGCATCTACTTTAGAAAGTGGGGTCCCATCAAGGTTCAGCGGCAGTGGAT
CTGGGACAGATTTCACTCTCACCATCAGCAGTCTGCAACCTGAAGATTTTGCAACTTACTACTGTCAA
CAGAGTTACAGTACCCCGTGGACGTTCGGCCAAGGGACCAAGCTGGAGATCAAA (SEQ ID NO: 670)
>N1wc11
CAGCCTGTGCTGACTCAGCCCCCCTCAGCGTCTGGGACCCCCGGGCAGAGGGTCACCATCTCTTGT
TCTGGAAGCAGCTCCAACCTCGGAATTAATACTGTAAACTGGTACCAGCAAGTCCCAGGAACGGCC
CCCAAACTCCTCATCTATAGTGATCATCAGCGGCCCTCAGGGGTCCCTGACCGATTCTCTGGCTCCA
AGTCTGCCACCTCAGCCTCCCTGGCCATCAGTGGGCTCCAGTCTGAGGATGAGGCTGATTATTACT
GTGCAGCATGGGATGACAGCCTGAATGGCTGGGTGTTCGGCGGAGGGACCAAGCTGACCGTCCTA
(SEQ ID NO: 671)
>N1wc13
CAGTCTGTGCTGACGCAGCCGCCCTCAGCGTCTGGGACCCCCGGGCAGAGGGTCACCATCCCTTG
TTCTGGAAGCAGCTCCAACATCGGAAGTAATTATGTCTACTGGTATCAGCAGCTCCCAGGAACGGCC
CCCAAACTCCTCATCTATAGGAATAATCAGCGGCCCTCAGGGGTCCCTGACCGATTCTCTGGCTCCA
AGTCTGGCACCTCAGCCTCCCTGGCCATCAGTGGACTCCGGTCCGAGGATGAGGCTGATTATTACT
GTGCAGCATGGGATGACAGCCTGAGTGGTCTGGTGTTCGGCGGAGGGACCAAGGTCACCGTCCTA
(SEQ ID NO: 672)
>N1wc15
TCCTATGTGCTGACTCAGCCACCCTCAGCGTCTGGGACCCCCGGGCAGAGGGTCACCGTCTCTTGT
TCTGGAAGCAGCTCCAGCATCGGAAGTAATACTGTAAACTGGTACCAGCAGCTCCCAGGAACGGCC
CCCAAGCTCCTCATCTATAGTAATAATCAGCGGCCCTCAGGGGTCCCTGACCGATTCTCTGGCTCCA
AGTCTGGCACCTCAGCCTCCCTGGCCATCAGTGGGCTCCAGTCTGAGGATGAGGCTGATTATTACT
GTGCAGCATGGGATGACAGCCTGAATGGTCTGGTATTCGGCGGAGGGACCAAGCTGACCGTCCTA
(SEQ ID NO: 673)
>N1wc16
CAGTCTGTGTTGACGCAGCCGCCCTCGGTGTCTGTGGCCCCAGGACAGACGGCCACACTTTCCTGT
GTGGGAGACGCCATTGAGAGAAAAAGTGTTCACTGGTACCAACAGAGGCCAGGCCAGGCCCCGGT
GCTGGTCGTCCATGATGATAGCGACCGGCCCTCAGGGATCCCTGACCGAATCTCTGGCTCCAACTC
TGGGAACACGGCCACCCTGACCATCACCAGGGTCGAAGTCGGGGATGAGGCCGACTACTTCTGTCA
GGTGTGGGACACTAGTAGTGGTCATGACGCTGTGGTCTTCGGCGGAGGGACCAAGCTGACCGTCC
TA (SEQ ID NO: 674)
>N1wc21
TCCTATGAGCTGACTCAGCCACCCTCGGTGTCAGTGTCCCCAGGACAAACGGCCAGGATCACCTGC
TCTGGAGATGCATTGCCAGACAAATATGTTCATTGGTACCAGCAGAAGTCAGGCCAGGCCCCTGTCC
TGGTCCTCTTCGACGACAGTAAGCGACCCTCCGGTATCCCTGAGAGATTCTCTGGCTCCAGCTCAG
GGACAGTGGCCACCTTGATTATCAGTGGGGCCCAGGTGGAGGATGAAGGTGACTTCTTCTGTTACT
CAACAGACAGCAGTGGTAATGAAAGGGTTTTCGGCGGAGGGACCAAGCTGACCGTCCTA
(SEQ ID NO: 675)
>N1wc24
CAGTCTGTGCTGACGCAGCCGCCCTCGGTGTCTGGGGCCCCCAGGCAGAGGGTCACCATCTCCTG
TTCTGGAAGCAGCTCCAACATCGAAAATAATATTGTAAACTGGTACCAGCAACTCCCAGGAGAGGCT
CCCAAACTCCTCATCTATTATGATAATCTGCTGCCCTCAGGGGTCTCTGACCGATTCTCTGGCTCCAA
GTCTGGCACCTCCGCCTCCCTGGCCATCAGTGGCCTCCAGTCTGGGGATGAGGCTGATTATTACTG
TGCAGCATGGGATGACAGCCTGAAGGCTGTGGTGTTCGGCGGAGGGACCAAGCTGACCGTCCTA
(SEQ ID NO: 676)
>N1wc72
CAGCCTGTGCTGACTCAGCCACCCTCAGCGTCTGGGACCCCCGGGCAGAGGGTCACCATCTCTTGT
TCTGGAAGCTACTCCAACATCGGAAGTAATTATGTCTACTGGTACCAGCAGTTCCCAGGAACGGCCC
CCAAACTCCTCATCTATACGAATAATCAGCGGCCCTCAGGGGTCCCTGACCGATTCTCTGGCTCCAA
GTCTGGCTCCTCAGCCTCCCTTGCCATCAGTGGGCTCCGGTCCGAGGATGAGGCTGATTATTACTG
TGCAGCATGGGATGACAGTGTGGGTGTCCTGTTATTCGGCGGGGGGACCAAGGTCACCGTCCTA
(SEQ ID NO: 677)
>N1wc79
CAGTCTGTGCTGACTCAGCCACCCTCAGCGTCTGGGACCCCCGGGCAGAGAGTCACCATCTCTTGT
TCTGGAAGCACCTCCAACATCGGAAAGAATACTGTAAACTGGTACCAGCAACTCCCAGCAACGGCC
CCCAAACTCCTCATCTATAGTGATAATCAGCGGCCCTCAGGGGTCCCTGACCGATTCTCTGGCTCCA
AGTCTGGCACCTCAGCCTCCCTGGCCATCAGTGGGCTCCAGTCTGAAGATGAGTCTGATTATTACTG
TGCAGCATGGGATGACAGCCTGGATGGCCTGGTATTCGGCGGAGGGACCAAGCTGACCGTCCTA
(SEQ ID NO: 678)
>N1wc82
CAGTCTGTGCTGACGCAGCCGCCCTCAGTGTCTGCGACCCCAGGACAGAAGGTCACCATCTCCTGC
TCTGGAAGCAACTCCAACATTGGGAATAATTATGTGTCCTGGTATCAACAACTCCCAGGAACAGCCC
CCAAACTTCTCATTTATGACAGTGATCAGCGACCCGCAGGGATTCCTGACCGATTCTCTGGCTCCAA
GTTAGGCACGTCAGCCACCCTGGGCATCACCGGACTCCAGACTGGGGACGAGGCCGATTATTACTG
CGCAACATGGGATGCCGGCCTGAGTGTTCTGATTTTCGGCGGAGGGACCAAGCTGACCGTCCTA
(SEQ ID NO: 679)
>N1wc102
CAGCCTGTGCTGACTCAGCCCCCCTCAGCGTCTGGGACCCCCGGGCAGAGGGTCGCCATCTCTTG
TTCTGGAAGCAGCTCCAACATCGGAAGTAATCCTGTAAACTGGTACCAACAACTCCCAGGAACGGCC
CCCAAGCTCCTCATCTATGGTGATAATCAGCGGCCCTCAGGGGTCCCTGACCGATTCTCTGGCTCC
AAGTCTGGCACCTCAGCCTCCCTGGCCGTCAGTGGGCTCCAGTCTGAGGATGAGGCTGATTATTTC
TGTGCAGCATGGGATGACAGCCTGAATGGTGTGGTCTTCGGCGGAGGGACCAAGCTGACCGTCCTA
(SEQ ID NO: 680)
>N1wc113
CAGGCTGTGCTGACTCAGCCGTCCTCAGTGTCTGGGGCCCCAGGGCAGAGGGTCACCATCTCCTG
CACTGGGAGCAGCTCCAACATCGGGGCAGGCAATGATGTACACTGGTATCGGCAGTTTCCAGGAAC
GGCCCCCAAACTCCTCATCTATGGAAATAACAAGCGGCCCTCAGGGGTCTCTGACCGATTCTCTGG
CTCCAAGTCTGGCACCTCAGCCTCCCTGGCCATCACTGGGCTCCAGGTTGAGGATGAGGCTGATTA
TTACTGCCAGTCCTATGACAGCAGCCTGGGTGGGTCGGACGTTTTCGGCGGAGGGACCAAGCTGAC
CGTCCTA (SEQ ID NO: 681)
>N1wc117
CAGTCTGTGCTGACTCAGCCACCCTCAGCGTCTGGGACCCCCGGGCAGAGGGTCACCATCTCCTGT
TCTGGAAGCAGCTCCAACATCGGAGGTAATACTGTAAACTGGTACCAGCAGCTCCCAGGAACGGCC
CCCAAACTCCTCATCTATGGTACTAATCAGCGGCCCTCAGGGGTCCCTGACCGATTCTCTGGCTCCA
AGTCTGGCACCTCAGCCTCCCTGGCCATCAGTGGGCTCCAGTCTGAGGATGAGGCTGATTATTACT
GTGCAGCATGGGATGACTTCCTGAATGGTAAGGTATTCGGCGGAGGGACCAAGCTGACCGTCCTA
(SEQ ID NO: 682)
>N1wc125
CAGTCTGTGCTGACTCAGCCTGCCTCCGTGTCTGGGTCTCCTGGACAGTCGATCACCATCTCCTGC
ACTGGAACCAGCAGTGACGTTGGTGGTTATAACTATGTCTCCTGGTACCAACAACACCCAGGCAAAG
CCCCCAAACTCATGATTTATGAGGGCAGTAAGCGGCCCTCAGGGGTTTCTAATCGCTTCTCTGGCTC
CAAGTCTGGCAACACGGCCTCCCTGACAATCTCTGGGCTCCAGGCTGAGGACGAGGCTGATTATTA
CTGCAGCTCATATACAACCAGGAGCACTCGAGTTTTCGGCGGAGGGACCAAGCTGACCGTCCTA
(SEQ ID NO: 683)
>N1wc127
TCCTATGTGCTGACTCAGCCACCCTCAACGTCTGGGGCCCCCGGGCAGAGGGTCACCATCTCTTGT
TCTGGGAGCAGCTCCAACATCGGAAGTAATGGTGTTAACTGGTACCACCACCTCCCAGGAACGGCC
CCCAAACTCCTCATCTACAGTAATTTTCATCGGCCCTCAGGGGTCCCGGACCGATTCTCTGGCTCCA
AGTCTGGCACCTCAGCCACCCTGGCCATCAGTGGGCTCCAGTCTGAGGATGAGGCTGATTATTACT
GTGCAGCATGGGATGACAGCCTGAATGATTATGTCTTCGGAGTTGGGACCAAGGTCACCGTCCTA
(SEQ ID NO: 684)
>N1wc129
CAGTCTGTGCTGACTCAGCCACCCTCAGCGTCTGGGACCCCCGGCCAGAGGGCCACCATCTCTTGT
TCTGGAAGCGGCTCCAACATCGGAAGAAATGCTGTTAACTGGTTCCAGCAGCTCCCAGGAGCGGCC
CCCAAACTCCTCATCTATAGTAATATTCACCGGCCCTCAGGGGTCCCGGACCGAATCTCTGGCTCCA
AGTCTGGCACCTCAGCCACCCTGGCCATCAGTGGGCTCCAGTCTGAGGATGAGGCTGATTATTACT
GTGCAACATGGGATGACAGCCTTAATGATTATGTCTTCGGAACTGGGACCAAGCTGACCGTCCTA
(SEQ ID NO: 685)
>N1wc130
CAGGCTGTGCTGACTCAGCCGTCCTCAGTGTCTGGGGCCCCAGGGCAGAGGGTCACCATCTCCTG
CACTGGGACCGGCTCCAACATCGGGGCAGGTTATGATGTACACTGGTACCAGCAGCTTCCAGGAAC
TGCCCCCAAACTCCTCATCTATGCTAACACCAATCGGCCCTCAGGGGTCCCTGACCGATTCTCTGGC
TCCAAGTCTGGCACCTCAGCCTCCCTGGCCATCACTGGGCTCCAGGCTGAGGATGAGGCTGATTAT
TACTGCCAGTCCTATGACAGCAGCCTGAGTGGTTCGGTGTTCGGCGGAGGGACCAAGGTCACCGTC
CTA (SEQ ID NO: 686)
>N1wc131
TCCTATGAGCTGACTCAGCCACCCTCAGCGTCTGGGACCCCCGGGCAGAGGGTCACCATCTCTTGT
TCTGGAACCAGCTCCAACATCGGAATTAATTATGTATACTGGTATCAGCAGCTCCCAAGAACGGCCC
CCAAACTCCTCATCTATTATAATAATCAACGGCCCTCAGGGGTCCCTGACCGATTCTCTGGCTCCAG
ATCTGGCACCTCAGCCTCCCTGGCCATCAGTGGGCTCCGGTCCGAGGATGAGGCTGATTATTACTG
TGCAGCATGGGATGACAGCCTGAGTGGTCTAGTCTTCGGAACTGGGACCAAGCTGACCGTCCTA
(SEQ ID NO: 687)
>N1wc132
CAGTCTGTGCTGACTCAGCCACCCTCAGCGTCTGGGACCCCCGGACAGAGGGTCACCATCTCTTGT
TCTGGAAGCAGCTCCAACATCGGAAGTAATCCTGTCAACTGGTACCAGCAGCTCCCAGGATCGGCC
CCCAAACTCCTCGTCTATAGTGATGATCAGCGGCCCTCAGGGGTCCCTGACCGATTCTCTGGCTCC
AAGTCTGGAGCCTCAGCCTCCCTGGCCATCAGTGGGCTCCAGTCTGAGGATGAGTCTGATTATTACT
GTGCAGCATGGGATGACAGCCAGAATGCTGTGGTTTTCGGCGGAGGGACCAAGGTCACCGTCCTA
(SEQ ID NO: 688)
>N1wc135
CTGCCTGTGCTGACTCAGCCACCCTCAGCGTCTGGGACCCCCGGGCAGAGGGTCATCATCTCTTGT
TCTGGAAGCAGCTCCAACATCGGAAGTAATTATGTATACTGGTACCAGCAGCTCCCAGGAACGGCC
CCCAAACTCCTCATCTATAGGAATAATCAGCGGCCCTCAGGGGTCCCTGACCGATTCTCTGGCTCCA
AGTCTGGCACCTCAGCCTCCCTGGCCATCAGTGGGCTCCGGTCCGAGGATGAGGCTGATTATCACT
GTGCAGCATGGGATGACAGCCTGAGTGGGGTGGTATTCGGCGGAGGGACCAAGCTGACCGTCCTA
(SEQ ID NO: 689)
>N1wc150
TCCTATGAGCTGACTCAGCCACCCTCAGCGTCTGGGACCCCCGGGCAGAGGGTCACCATCTCTTGT
TCTGGAAGCGGCTCCAACATCGGGAAGAATACTGTAAACTGGTATCAGCAGCTCCCAGGGGCGGCC
CCCAAACTCCTCATCTATAGTAATAATGAGCGTCCCTCAGGGGTCCCTGGCCGGTTCTCTGGCTCCA
AGTCTGGCACCTCAGCCTCCCTGGCCATCAGTGGGCTCCAGTCTGAGGATGAGACTGATTATTATTG
TGCAGCCTGGGATGACAGCCTGAATGATATGGTCTTCGGCGGAGGGACCAAGGTCACCGTCCTA
(SEQ ID NO: 690)
>N1wc164
CAGGCTGTGCTGACTCAGCCGTCCTCAGTGTCTGGGGCCCCAGGGCAGAGGATCACCATCTCCTG
CACTGGGACCAGCTCCAACATCGGGGCAGGTTATGATGTAGAGTGGTACCAGCACCTTCCAGGAAC
AGCCCCCAAACTCCTCATCTATCGTAACAGCAATCGGCCCTCAGGGGTCCCTGATCGATTCTCTGCC
TCCAAGTCTGGCACCTCAGCCTCCCTGGTCATCACTGGACTCCAGGCTGAAGATGAGGCTGATTATT
ACTGCCAGTCCTATGACACCAGGCTGAGTGATTCTGTGATTTTCGGCGGAGGGACCAAGCTGACCG
TCCTA (SEQ ID NO: 691)
>N1wc170
TCCTATGAGCTGACTCAGCCACCCTCGGTGTCAGTGGCCCCAGGGCAGACGGCCAGGATTACCTGT
GGGGGAGAAAACATTGGAACTAAAAGTGTGCACTGGTACCAGCAGAAGTCAGGCCAGGCCCCTGTG
CTAGTCATCTATGAGGACAAGAAACGACCCTCCGGGATCCCTGACAGATTCTCTGGCTCCAGCTCAG
GGACAATGGCCACCTTGACTATCACTGGGGCCCAGGTGGACGATGAAGCTGACTACTACTGTTACT
CAGCAGACAACACTGGTGATCAGAGAATGTTCGGCGGAGGGACCAAGCTGACCGTCCTA
(SEQ ID NO: 692)
>N1wc177
CAGGCTGTGCTGACTCAGCCGTCTTCAGCGTCTGGGACCCCCGGGCAGAGGGTCATCATCTCTTGT
TCTGGAAGCAGCTCCAACATCGGAAAGTACATTGTTAATTGGTATCAGCAACTCCCAGGAGCGGCCC
CCAAACTCCTCATCTATACTTATAATGAGCGGCCCTCAGGGGTCCCTGCCCGCTTCTCTGGCTCCAC
GTCTGGCACGTCAGCCTCCCTGGCCATCACTGGGCTCCAGTCTGAAGATGAGGCTGATTATTATTGT
GCATCATGGGATGACAGCCTGAATGTCGTGGTCTTCGGCGGAGGGACCAAGCTGACCGTCCTA
(SEQ ID NO: 693)
>N1wc182
CAGTCTGTCGTGACGCAGCCGCCCTCAGCGTCTGGGACCCCCGGGCAGAGGGTCACCATCTCTTG
TTCTGGAAGCAGCTCCAACATCGGAAGTAATACTGTAAACTGGTACCAGCAGCTCCCAGGAATGGCC
CCCAAACTCCTCATCTATAGTAATAATCAGCGGCCCCCAGGGGTCCCTGACCGATTCTCTGGCTCCA
AGTCTGGCACCTCAGCCTCCCTGGCCATCAGTGGGCTCCAGTCTGAGGATGAGGCTGATTATTACT
GTGCAGCATGGGATGACAGCCTGAATGGGAATTGGGTTTTCGGCGGAGGGACCAAGCTGACCGTC
CTA (SEQ ID NO: 694)
>N1wc197
CAGTCTGTGCTGACTCAGCCACCCTCAGCGTCTGGGACCCCCGGGCAGAGGGTCACCATCTCTTGT
TCTGGAAGCAGCTCCAACATCGGAAGTAATTATGTATATTGGTACCAGAAGTTCCCAGGAACGGCCC
CCAAACTCCTCATCTATACGAATAATCAGCGGCCCTCAGGGGTCCCTGACCGATTTTCTGGCTCCAA
GTCTGGCACCTCAGCCTCCCTGGCCATCAGTGGGCTCCGGTCCGAGGATGAGGCTGATTATTACTG
TGCAGCATGGGATGACAGCCTGAGTGGTGTGGTATTCGGCGGAGGGACCAAGGTCACCGTCCTA
(SEQ ID NO: 695)
>N1wc225
CAGTCTGTCGTGACGCAGCCGCCCTCAGTGTCTGGGGCCCCAGGGCAGAGGGTCACCATCTCCTG
CACTGGGAGCAGCTCCAACATCGGGGCAGGTTATGATGTACACTGGTACCAGCAGCTTCCAGGAAC
AGCCCCCAAACTCCTCATCTATGGTAACAGCAATCGGCCCTCAGGGGTCCCTGACCGATTCTCTGG
CTCCAAGTCTGGCACCTCAGCCTCCCTGGCCATCACTGGGCTCCAGGCTGAGGATGAGGCTGATTA
TTACTGCCAGTCCTATGACAGCAGCCTGAGTGGTTCGGTGTTCGGCGGAGGGACCAAGCTGACCGT
CCTA (SEQ ID NO: 696)
>N1wc239
CAGTCTGTGTTGACGCAGCCGCCCTCAGCGTCTGGGACCCCCGGGCAGAGGGTCGCCATCTCTTG
TTCTGGCAGCCACTCCAACATCAGAAGTAATGATGTTTATTGGTACCAGCAGTTTCCAGGAACGGCC
CCCAAACTCCTCATCTATAGTAATGATCAGCGGCCCTCAGGGGTCTCTGACCGATTCGCTGGCTCCA
AGTCTGGCACCTCAGCCTCCCTGGCCATCAGCGGACTCCGGTCCGAGGATGAGTCTGATTATTATT
GTGCAACATGGGATGACAGCCTGAGTGGCGTGGTCTTCGGCGGAGGGACCAAGCTGACCGTCCTA
(SEQ ID NO: 697)
>N1wc241
CAGTCTGTGCTGACTCAGCCACCCTCAGCGTCTGGGACCCCCGGGCAGAGGGTCACCATCTCTTGT
TCTGGAAGCTTCTCCAACATCGGAAGTAATAATGTATACTGGTACCAGCAGCTCGCAGGAACGGCCC
CCAAACTCCTCATCTATAGGAATAATGAGCGGCCCTCAGGGGTCCCTGACCGATTCTCTGGCTCCAA
GTCTGGCACCTCAGCCTCCCTGGCCATCAGTGGGCTCCGGTCCGAGGATGAGGCTGATTATTACTG
TGCAGTACGGGATGACAGCCTGAGTGGTCATTATGTCTTCGGATCTGGGACCAAGGTCACCGTCCTA
(SEQ ID NO: 698)
>N1wc248
CAGTCTGTGTTGACGCAGCCGCCCTCAGCGTCTGGGACCCCCGGGCAGAGGGTCACCATCTCCTG
TTCTGGAAGCAGCTCCAATATCGGAAGTAATTCTGTATACTGGTACCAGCAGTTCCCAGGAACGGCC
CCCAAACTCCTCATCTATAGGAATAATCAGCGGCCCTCAGGGGTCCCTGACCGATTCTCTGGTTCCA
AGTCTGACACCTCAGCCTCCCTGGCCATCAGTGGGCTCCGGTCCGAGGATGAGGCTGATTATTACT
GTGCAACATGGGATGACAGCCTGAGTGGTGTGGTATTCGGCGGAGGGACCAAGCTGACCGTCCTA
(SEQ ID NO: 699)
>N1wc266
CAGGCTGTGCTGACTCAGCCGTCCTCCGTGTCTGGGACCCCCGGCCAGAGGGTCACCATGTCTTGT
TCTGGAGGCAGGTCCAACATCGGAAGTAATGCTGTCAACTGGTACCAACAGTTCCCAGGAACGGCC
CCCAAACTCCTCATGTATAATACTAATCAGCGGCCCTCAGGGGTCCCTGGCCGATTCTCTGGCTCCA
AGTCTGGCGCCTCAGCCTCCCTGGCCATCAGTGGCCTCCAGTCTGATGATGAGGCTGATTATTACT
GTTCGTCTTGGGATGACAGCCTCAATGGTCTGGAGTTCGGCGGAGGGACCAAGCTGGAGATCAAA
(SEQ ID NO: 700)
>N1wc281
CAGTCTGTGCTGACTCAGCCACCCTCAGTGTCCGTGTCCCCAGGACAGACAGCCAGCATCACCTGC
TCTGGAGATAAATTGGGAGATAAATATACTTTCTGGTATCAGCAGAAGCCAGGCCAGTCCCCTGTGC
TGATCATCTATCAAGATACCAAGCGGCCCTCAGGGATCCCAGAGCGATTCTCTGGCTCCAACTCTGG
GAACACAGCCACTCTGACCATCAGCGGGACCCAGGCTATGGATGAGGCTGACTATTACTGTCAGGC
GTGGGATAGTAATACTGATCATGTGATATTCGGCGGAGGGACCAAGCTGACCGTCCTA
(SEQ ID NO: 701)
>N1wc289
CAGGCTGTGCTGACTCAGCCGTCTTCCCTCTCTGCATCTCCTGGAGCATCAGCCAGTCTCACCTGCA
CCTTGCGCAGTGACATCAATCTTGAGACCTCCAGGATTTATTGGTTTCAACAAAAGCCAGGGAGTCC
TCCCCGCTATCTCTTGAGGTACCAGTCAGACTCAGATAATAACCTGGACTCTGGAGTCCCCAGCCGC
TTCTCTGGATCCAAAGATGCTTCGGCCAACGCAGGAATTTTGCTCATCTCTGGGGTCCAGTCTGAGG
ATGAGGCTGATTATCATTGTATGATTTGGCACAGCGGCGGTTCGGTTTTCGGCGGAGGGACCAAGG
TCACCGTCCTA (SEQ ID NO: 702)
>N1wc295
CAGTCTGTGTTGACGCAGCCGCCCTCAGCGTCTGGGACCCCCGGGCAGAGGGTCACCATCTCTTGT
TCTGGAAACAGGTCCAACATCGGAAGTAATACTGTAAACTGGTACCAGCAACTCCCAGGAACGGCC
CCCAAACTCCTCATCTATAGTAATGATCAGCGGCCCTCAGGGGTCCCTGACCGATTCTCTGGCTCCA
AGTCTGGCACCTCAGCCTCCCTGACCATCAGTGGGCTCCAGTCTGACGATGAGGCTGATTTTTACTG
TGCAGCATGGGATGATAATCTGAATGGTTATGTCTTCGGAAGTGGGACCAAGGTCACCGTCCTA
(SEQ ID NO: 703)
>N1wc301
CAGTCTGTCGTGACGCAGCCGCCCTCAGCGTCTGGGACCCCCGGGCAGAGGGTCACCATCTCTTG
TTCTGGAAGCAGCTCCAACATCGGAAAGAATAATGTATACTGGTACCAGCAGCTCCCAGGAGCGGC
CCCCAAACTCCTCATCTATCGGAATCATGAGCGGGCCTCAGGGGTCCCTGACCGATTCTCTGGCTC
CAAGTCTGGCACCTCAGCCTCCCTGGCCATCAGTGGGCTCCAGTCTGAGGATGAGGCTGATTATTA
CTGTGCAGCACGGGATGACAGCCTGAACGGTTATGTCTTCGGAACTGGGACCAAGGTCACCGTCCTA
(SEQ ID NO: 704)
>N1wc319
CAGTCTGTGCTGACTCAGCCACCCTCATCATCTGGGACCCCCGGGCAGAGGGTCACCATCTCTTGT
TCTGGAAGCAGCTCCAACATCGGAAGTAACTCTGTAAACTGGTACCAACAACTCCCAGGGGCGGCC
CCCAAACTCCTCATCTATAGTGATAATCAGCGGCCCTCAGGGGTCCCTGACCGATTCTCTGCCTCCA
AGTCTTACACCTCAGCCTCCCTGGCCATCAGTGGGCTCCAGTCTGGGGATGAGGCTGATTATTTCTG
TGCAACATGGGATGACAGCCTGACTGGTCTAGTCTTCGGCGGAGGGACCAAGCTGACCGTCCTA
(SEQ ID NO: 705)
>N1wc336
CAGGCTGTGCTGACTCAGCCGTCCTCAGCGTCTGGGACCCCCGGGCAGAGGGTCACCATCTCTTGT
TCTGGAAGCTACTCCAACATCGGAAGTAATTATGTATACTGGTACCAGCAGCTCCCAGGAACGGCCC
CCAAACTCCTCATCTATACGAATAATCAGCGGCCCTCAGGGGTCCCTGACCGATTCTCTGGCTCCAA
GTCTGGCACCTCAGCCTCCCTTGTCATCAGTGGGCTCCGGTCCGACGATGAAGGTGATTATTACTGT
GCAGCATGGGATGACAGTGTGAGTGTCCTGTTATTCGGCGGGGGGACCAAGCTGACCGTCCTA
(SEQ ID NO: 706)
>N1wc338
CAGTCTGTGTTGACGCAGCCGCCCTCAGCGTCTGGGACCCCCGGGCAGAGGGTCACCATCTCTTGT
TCTGGAAGCAGTTCCAACATCGGAACTTACACTGTAAACTGGTACCAGCAGCACCCAGGAACGGCC
CCCAAGCTCCTCATCTATAGTAATGATCAGCGGCCCTCAGGGGTCCCTGATCGATTCTCTGGCTCCA
GGTCTGGCACCTCAGCCTCCCTGGCCATCAGTGGACTCCGGTCTGAGGATGAGGCTGATTATTACT
GTGCAGCATGGGATGACAGCCTACATGGCTATGTCTTCAGAACTGGGACCAAGCTGACCGTCCTA
(SEQ ID NO: 707)
>N1wc403
CAGGCTGTGCTGACTCAGCCGTCCTCAGCGTCTGGGACCCCCGGGCAGAGGGTCACCATCTCTTGT
CCTGGAAGCAGCTCCAACATCGGAAGTAATACTGTAAACTGGTACCAGCAGCTCCCAGGAACGGCC
CCCAAACTCCTCATCTATACTAATAATCAGCGGCCCTCAGGGGTCCCTGACCGATTCTCTGCCTCCA
AGTCTGGCACCTCAGCCTCCCTGGCCATCAGTGGGCTCCAGTCTGAGGATGAGGCTGATTATTACT
GTGCAGCATGGGATGACAGCCTGAATGGTTATGTCTTCGGAACTGGGACCAAGCTGACCGTCCTA
(SEQ ID NO: 708)
>N1wc441
TCCTATGAGCTGACTCAGCCACCCTCGGTGTCAGTGTCCCCAGGACAAACGGCCAGGATCACCTGC
TCTGGAGATGCATTGCCAGACAAATATGTTCATTGGTACCAGCAGAAGTCAGGCCAGGCCCCTGTCC
TGGTCCTCTTCGACGACAGTAAGCGACCCTCCGGTATCCCTGAGAGATTCTCTGGCTCCAGCTCAG
GGACAGTGGCCACCTTGATTATCAGTGGGGCCCAGGTGGAGGATGAAGGTGACTTCTTCTGTTACT
CAACAGACAGCAGTGGTAATGAAAGGGTTTTCGGCGGAGGGACCAAGCTGACCGTCCTA
(SEQ ID NO: 709)
>N1wc476
CAGGCTGTGCTGACTCAGCCGTCCTCAGTGTCTGGGACCCCCGGGCAGACGGTCACCATCTCTTGT
TCTGGCAGCAACTCCAACATCGGAAATAATTATGTATACTGGTACCAACAATTGCCAGGGTCGGCCC
CCAAAGTCGTCATCTACAGGAATCATCAGCGGCCCTCAGGGGTCCCTGACCGATTCTCTGGCTCCA
AGTCTGGCACCGCCGCCTCCCTGGCCATCAGTGGACTCCGGTCCGAAGATGAAGCTGATTATTATT
GTGCATCATGGGATGACAGCCTGAGTGGTCTGGTTTTCGGCGGGGGGACCAAGCTGACCGTCCTA
(SEQ ID NO: 710)
>N1wc516
CAGCCTGTGCTGACTCAGCCCCCCTCAGCGTCTGGGACCCCCGGGCAGAGGGTCACCATCTCTTGT
TCTGGAAGCAGCTCCAACATCGGAAGTAATACTGTAAACTGGTACCAGCAGCTCCCAGGAACGGCC
CCCAAACTCCTCATCTATAGTAATAATCAGCGGCCCTCAGGGGTCCCTGACCGATTCTCTGGCTCCA
AGTCTGGCACCTCAGCCTCCCTGGCCATCAGTGGGCTCCAGTCTGAGGATGAGGCTGATTATTACT
GTGCAGCATGGGATGACAGCCTGAATGATGTGGTATTCGGCGGAGGGACCAAGCTGACCGTCCTA
(SEQ ID NO: 711)
>N1wc535
CAGTCTGTGCTGACGCAGCCGCCCTCAGCGTCTGGGACCCCGGGGCAGAGGGTCACCCTCTCTTG
TTCTGGAAGCAGCTCCAACATCGGAAGTAATACTGTAAACTGGTACCAGCAGCTCCCAGGAACGGC
CCCCAAAGTCCTCATCTATAGTGATAATCAGCGGCCCTCAGGGGTCCCTGACCGATTCTCTGGCTCC
AAGTCTGGCACCTCAGCCTCCCTGGCCATCAGTGGGCTCCAGTCTGACGATGAGGCTGTTTATTACT
GTGCAGGTTGGGATGACAGCCTGAATGCTTATCTCTTCGGAACTGGGACCAAGGTCACCGTCCTA
(SEQ ID NO: 712)
>N1wc540
CAGGCTGTGCTGACTCAGCCGTCCTCAGCGTCTGGGACCCCCGGGCAGAGGGTCACCATCTCTTGT
TCTGGAAGCAGCTCCAACATCGGAAGTTATACCGTAAACTGGTACCAGCAGGTCCCAGGAACGGCC
CCCAAACTCCTCATCTATAGTAATAATCAGCGGCCCTCAGGGGTCCCTGAGCGATTCTCTGGCTCCA
AGTCTGGCACCTCAGCCTCCCTGGCCATCAGTGGGCTCCAGTCTGAGGATGAGGCTGATTATTACT
GTGCAGCATGGGATGACAGCCTGAGTGATGTGCTATTCGGCGGAGGGACCAAGCTGACCGTCCTA
(SEQ ID NO: 713)
>N1wc558
CTGCCTGTGCTGACTCAGCCCCCCTCAGCGTCTGGGACCCCCGGGCAGAGGGTCACCATCTCTTGT
TCTGGAAGCAGCTCCAACATCGGAAGTAATTCTGTAAACTGGTACCAGCAACTCCCCGGAACGGCC
CCCAAACTCCTCATCTATTCTAATAATCAGCGGCCCTCAGGGGTCCCTGACCGATTCTCTGGCTCCC
AGTCTGGCACCTCAGCCTCCCTGGCCATCAGTGGACTCCGGTCCGAGGATGAGGCTGATTATTACT
GTGCAGCATGGGATGACAGCCTGAATAATTTGGTGTTCGGCGGAGGGACCAAGCTGACCGTCCTA
(SEQ ID NO: 714)
>N1wc570
CAGTCTGTGCTGACGCAGCCGCCCTCAGCGTCTGCGACCCCCGGGCAGACGGTCACCATCTCTTGT
TCTGGAAGCACCTCCAACATCGGAAATAATAATGTAAACTGGTACCAGCAGGTCCCAGGAACGGCC
CCCAAACTCCTCATGTACAGTAATAATCAGCGGCCCTCAGGGGTCCCTGACCGATTCTCTGGCTCCA
GGTCTGGCACCTCAGCCTCCCTGGCCATCACTGCGCTCCAGTCTGAGGATGAGGCTGATTATTACT
GTGCAACATGGGATGACAGCCTGAGTGCTTGGGTATTCGGCGGAGGGACCAAGGTCACCGTCCTA
(SEQ ID NO: 715)
>N1wc572
CTGCCTGTGCTGACTCAGCCCCCCTCAGCGTCTGGGACCCCCGGACAGAGGGTCACCATCTCTTGT
TCTGGAAGCAGCTCCAACATCGGAAGTAATCCTGTCAACTGGTACCAGCAGCTCCCAGGATCGGCC
CCCAAACTCCTCGTCTATAGTGATGATCAGCGGCCCTCAGGGGTCCCTGACCGATTCTCTGGCTCC
AAGTCTGGAGCCTCAGCCTCCCTGGCCATCAGTGGGCTCCAGTCTGAGGATGAGTCTGATTATTACT
GTGCAGCATGGGATGACAGCCTGAATGCTGTGGTTTTCGGCGGAGGGACCAAGGTCACCGTCCTA
(SEQ ID NO: 716)
>N1wc586
GACATCGTGATGACCCAGTCTCCTTCCACCCTGTCTGCATCTGTAGGAGACAGAGTCACCATCACTT
GCCGGGCCAGTCAGGGTATTAGTAGCTGGTTGGCCTGGTATCAGCAGAAACCAGGGAGAGCCCCT
AAGGTCTTGATCTATAAGGCATCTACTTTAGAAAGTGGGGTCCCATCAAGGTTCAGCGGCAGTGGAT
CTGGGACAGATTTCACTCTCACCATCAGCAGTCTGCAACCTGAAGATTTTGCAACTTACTACTGTCAA
CAGAGTTACAGTACCCCGTGGACGTTCGGCCAAGGGACCAAGCTGGAGATCAAA (SEQ ID NO: 717)
>N1wc614
CAGTCTGTGCTGACGCAGCCGCCCTCAGCGTCTGGGACCCCCGGGCAGAGGGTCACCATATCTTG
TTCTGGAAGCAGCTCCAACATCGGAAGTAATACTGTAAACTGGTACCAGCAGCTCCCAGGAACGGC
CCCCAAACTCCTCATCTATAGTAATAATCAGCGGCCCTCAGGGGTCCCTGACCGATTCTCTGGCTCC
AAGTCTGGCACCTCAGCCTCCCTGGCCATCAGTGGGCTCCAGTCTGAGGATGAGGCTGATTATTAC
TGTGCAGCATGGGATGACAGCCTGAATGGTCTGGTGTTCGGCGGAGGGACCAAGGTCACCGTCCTA
(SEQ ID NO: 718)
>N1wc621
CAGGCTGTGCTGACTCAGCCGTCCTCAGTGTCTGGGGCCCCAGGGCAGACGGTCACCATCTCCTG
CGCTGGGAGCAGCTCCAACCTCGGGGCAGGTTATGATGCACACTGGTATCAACACCTTCCAGGAGC
AGTCCCCAGACTCCTCATTTATAATAATAGTAAACGCCCCTCAGGGGTCCCTGACCGATTCTCTGCC
TCCAAGTCCGGCTCCTCAGCCTCCCTGGCCATCACTGGGCTCCAGCCTGAGGATGAGGCTGATTAC
TACTGCCAGACCTATGACACCAGACTGAGCGGATCGATGCTATTCGGCGGAGGGACCAAGGTCACC
GTCCTA (SEQ ID NO: 719)
>N1wc624
CAGTCTGTGCTGACGCAGCCGCCCTCAGCGTCTGGGACCCCCGGGCAGGGGGTCACCATCTCTTG
TTCTGGAAGCAGCTCCAACATCGGAAGTAATACTGTAAACTGGTACCAGCAGCTCCCAGGAACGGC
CCCCAAACTCCTCATCTATAGTAATAATCAGCGGCCCTCAGGGGTCCCTGACCGATTCTCTGGCTCC
AAGTCTGGCACCTCAGCCTCCCTGGCCATCAGTGGGCTCCAGTCTGAGGATGAGGCTGATTATTAC
TGTGCTGCATGGGATGACAGCCTGAATGGTAAAGTCTTCGGAACTGGGACCAAGGTCACCGTCCTA
(SEQ ID NO: 720)
>N1wc633
CAGGCTGTGCTGACTCAGCCGTCCTCAGCGTCTGGGACCCCCGGGCAGAGGGTCACCATCTCTTGT
TCTGGAAGCAGCTCCAACATCGGAAGTTATACTGTAAACTGGTACCAGCAGGTCCCAGGAACGGCC
CCCAAACTCCTCATCTATAGTAATAATCAGCGGCCCTCAGGGGTCCCTGAGCGATTCTCTGGCTCCA
AGTCTGGCACCTCAGCCTCCCTGGCCATCAGTGGGCTCCAGTCTGAGGATGAGGCTGATTATTACT
GTGCAGCATGGGATGACAGCCTGAGTGATGTGCTATTCGGCGGAGGGACCAAGGTCACCGTCCTA
(SEQ ID NO: 721)
>N1wc39
GACATCCAGTTGACCCAGTCTCCTTCCTCCGTGTCTGCATCTATAGGCGACACAGTCACCATCACTT
GTCGGGCGAGCCAGAATATTAACAAGTGGTTAGCCTGGTATCAGCAGAAACCAGGGAAAGCCCCTT
ACCTCCTGATCTCTACTGCTTCCAATTTGCAAAGTGGGGTCCCATCAAGGTTCAGCGGCACTGGGTC
TGGGACAGCTTTCACTCTCACCATCAGCCGCGTGCAGCCTGAAGATTTTGCAAATTACTATTGTCAA
CAGGCTTTCAGTTTCCCCATCACCTTCGGCCAAGGGACACGACTGGAGATTAAA
(SEQ ID NO: 722)
>N1wc45
TCGTCTGAGCTGACTCAGGACCCTGCTGTGTCTGTGGCCTTGGGACAGACAGTCAGGATCACATGC
CAAGGAGACAGCCTCAGAAGCTATTATGCAAGCTGGTACCAGCAGAAGCCAGGACAGGCCCCTGTA
CTTGTCATCTATGGTAAAAACAACCGGCCCTCAGGGATCCCAGACCGATTCTCTGGCTCCAGCTCAG
GAAACACAGCTTCCTTGACCATCACAGGGGCTCAGGCGGAAGATGAGGCTGACTATTACTGTAACTC
CCGGGACAGCAGTGGTAACCATGTGGTATTCGGCGGAGGGACCAAGCTGACCGTCCTA
(SEQ ID NO: 723)

C. CDR (Aminoacid sequence)
Heavy Chain

| Clone Name | HC-CDR1 | HC-CDR2 | HC-CDR3 |
|---|---|---|---|
| hN1S3-001 | GYNFTGYY | INPNSGG | ATDSFDY (SEQ ID NO: 209) |
| | (SEQ ID | (SEQ ID | |
| | NO: 207) | NO: 208) | |
| hN1S3-002 | GYTFTDYY | INPNNGG | ARDLLPYYGDYRADKIDDFDI (SEQ ID |
| | (SEQ ID | (SEQ ID | NO: 725) |
| | NO: 268) | NO: 724) | |
| hN1S3-003 | GFTFTNSA | IVVGSGN | VADTVY (SEQ ID NO: 215) |
| | (SEQ ID | (SEQ ID | |
| | NO: 726) | NO: 214) | |
| hN1S3-006 | GYNFTGYY | INPNSGG | ATDSFDY (SEQ ID NO: 209) |
| | (SEQ ID | (SEQ ID | |
| | NO: 207) | NO: 208) | |
| hN1S3-007 | GYNFTGYY | INPNSGG | ATDSFDY (SEQ ID NO: 209) |
| | (SEQ ID | (SEQ ID | |
| | NO: 207) | NO: 208) | |
| hN1S3-023 | GYNFTGYY | INPNSGG | ATDSFDY (SEQ ID NO: 209) |
| | (SEQ ID | (SEQ ID | |
| | NO: 207) | NO: 208) | |
| hN1S3-030 | GYNFTGYY | INPNSGG | ATDSFDY (SEQ ID NO: 209) |
| | (SEQ ID | (SEQ ID | |
| | NO: 207) | NO: 208) | |
| hN1S3-032 | GYNFTGYY | INPNSGG | ATDSFDY (SEQ ID NO: 209) |
| | (SEQ ID | (SEQ ID | |
| | NO: 207) | NO: 208) | |
| hN1S3-035 | GYNFTGYY | INPNSGG | ATDSFDY (SEQ ID NO: 209) |
| | (SEQ ID | (SEQ ID | |
| | NO: 207) | NO: 208) | |
| hN1S3-036 | GYNFTGYY | INPNSGG | ATDSFDY (SEQ ID NO: 209) |
| | (SEQ ID | (SEQ ID | |
| | NO: 207) | NO: 208) | |
| hN1S3-038 | GYNFTGYY | INPNSGG | ATDSFDY (SEQ ID NO: 209) |
| | (SEQ ID | (SEQ ID | |
| | NO: 207) | NO: 208) | |
| hN1S3-040 | GFTFTSSA | IVVGSGN | VADTVY (SEQ ID NO: 215) |
| | (SEQ ID | (SEQ ID | |
| | NO: 213) | NO: 214) | |
| hN1S3-053 | GYNFTGYY | INPNSGG | ATDSFDY (SEQ ID NO: 209) |
| | (SEQ ID | (SEQ ID | |
| | NO: 207) | NO: 208) | |
| hN1S3-055 | GYNFTGYY | INPNSGG | ATDSFDY (SEQ ID NO: 209) |
| | (SEQ ID | (SEQ ID | |
| | NO: 207) | NO: 208) | |
| hN1S3-147 | GFTFTSSA | IVVGSGN | VADTVY (SEQ ID NO: 215) |
| | (SEQ ID | (SEQ ID | |
| | NO: 213) | NO: 214) | |
| hN1S3-074 | GYNFTGYY | INPNSGG | ATDSFDY (SEQ ID NO: 209) |
| | (SEQ ID | (SEQ ID | |
| | NO: 207) | NO: 208) | |
| hN1S3-077 | GYGFSNYY | INPSDGS (SEQ | AREKANSMYFDY (SEQ ID NO: 729) |
| | (SEQ ID | ID NO: 728) | |
| | NO: 727) | | |
| hN1S3-082 | GFTFTSSA | IVVGSGN | VADTVY (SEQ ID NO: 215) |
| | (SEQ ID | (SEQ ID | |
| | NO: 213) | NO: 214) | |
| hN1S3-083 | GDSISSSSAT | TYYRSKWY | ARQLGGRIEF (SEQ ID NO: 732) |
| | (SEQ ID | (SEQ ID | |
| | NO: 730) | NO: 731) | |
| hN1S3-087 | GFTFTSYF | INPSRGN | ARDRLGGLLDYYDSSDSSKNFDAFDF |
| | (SEQ ID | (SEQ ID | (SEQ ID NO: 212) |
| | NO: 210) | NO: 211) | |
| hN1S3-088 | GFTFSSYA | ISGSGGS | ATEPPFDI (SEQ ID NO: 735) |
| | (SEQ ID | (SEQ ID | |
| | NO: 733) | NO: 734) | |
| hN1S3-091 | GYNFTGYY | INPNSGG | ATDSFDY (SEQ ID NO: 209) |
| | (SEQ ID | (SEQ ID | |
| | NO: 207) | NO: 208) | |
| hN1S3-092 | GYNFTGYY | INPNSGG | ATDSFDY (SEQ ID NO: 209) |
| | (SEQ ID | (SEQ ID | |
| | NO: 207) | NO: 208) | |
| hN1S3-095 | GYNFTGYY | INPNSGG | ATDSFDY (SEQ ID NO: 209) |
| | (SEQ ID | (SEQ ID | |
| | NO: 207) | NO: 208) | |
| hN1S3-096 | GYNFTGYY | INPNSGG | ATDSFDY (SEQ ID NO: 209) |
| | (SEQ ID | (SEQ ID | |
| | NO: 207) | NO: 208) | |
| hN1S3-107 | GYTFTDYF | INPNTGG | ARGAYDYVWGSHRYVADAFDI (SEQ |
| | (SEQ ID | (SEQ ID | ID NO: 738) |
| | NO: 736) | NO: 737) | |
| hN1S3-112 | GYNFTGYY | INPNSGG | ATDSFDY (SEQ ID NO: 209) |
| | (SEQ ID | (SEQ ID | |
| | NO: 207) | NO: 208) | |
| hN1S3-124 | GYNFTGYY | INPNSGG | ATDSFDY (SEQ ID NO: 209) |
| | (SEQ ID | (SEQ ID | |
| | NO: 207) | NO: 208) | |
| hN1S3-127 | GYNFTGYY | INPNSGG | ATDSFDY (SEQ ID NO: 209) |
| | (SEQ ID | (SEQ ID | |
| | NO: 207) | NO: 208) | |
| hN1S3-133 | GFTFTSSA | IVVGSGN | VADTVY (SEQ ID NO: 215) |
| | (SEQ ID | (SEQ ID | |
| | NO: 213) | NO: 214) | |
| hN1S3-135 | GYNFTGYY | INPNSGG | ATDSFDY (SEQ ID NO: 209) |
| | (SEQ ID | (SEQ ID | |
| | NO: 207) | NO: 208) | |
| hN1S3-136 | GYNFTGYY | INPNSGG | ATDSFDY (SEQ ID NO: 209) |
| | (SEQ ID | (SEQ ID | |
| | NO: 207) | NO: 208) | |
| hN1S3-141 | GYNFTGYY | INPNSGG | ATDSFDY (SEQ ID NO: 209) |
| | (SEQ ID | (SEQ ID | |
| | NO: 207) | NO: 208) | |
| hN1S3-151 | GYNFTGYY | INPNSGG | ATDSFDY (SEQ ID NO: 209) |
| | (SEQ ID | (SEQ ID | |
| | NO: 207) | NO: 208) | |
| hN1S3-153 | GFTFGNYV | ITEGGGDF | AKSGGDDNSNYNFEY (SEQ ID |
| | (SEQ ID | (SEQ ID | NO: 741) |
| | NO: 739) | NO: 740) | |
| hN1S3-177 | GYNFTGYY | INPNSGG | ATDSFDY (SEQ ID NO: 209) |
| | (SEQ ID | (SEQ ID | |
| | NO: 207) | NO: 208) | |
| hN1S3-181 | GYNFTGYY | INPNSGG | ATDSFDY (SEQ ID NO: 209) |
| | (SEQ ID | (SEQ ID | |
| | NO: 207) | NO: 208) | |
| hN1S3-182 | GYNFTGYY | INPNSGG | ATDSFDY (SEQ ID NO: 209) |
| | (SEQ ID | (SEQ ID | |
| | NO: 207) | NO: 208) | |
| hN1S3-184 | GYNFTGYY | INPNSGG | ATDSFDY (SEQ ID NO: 209) |
| | (SEQ ID | (SEQ ID | |
| | NO: 207) | NO: 208) | |
| hN1S3-190 | GFTFTSYF | INPSRGN | ARDRLGGLLDYYDSSDSSKNFDAFDF |
| | (SEQ ID | (SEQ ID | (SEQ ID NO: 212) |
| | NO: 210) | NO: 211) | |
| hN1S3-191 | GYNFTGYY | INPNSGG | ATDSFDY (SEQ ID NO: 208) |
| | (SEQ ID | (SEQ ID | |
| | NO: 207) | NO: 208) | |
| hN1S3-108 | GYTFTDYY | INPNTGG | ARGAYDYVWGSHRYVADAFDI (SEQ |
| | (SEQ ID | (SEQ ID | ID NO: 738) |
| | NO: 268) | NO: 737) | |
| hN1S3-130 | GYTFTDFY | INSKSGD (SEQ | ARGAYDYVWGSSRYVADAFDI (SEQ |
| | (SEQ ID | ID NO: 743) | ID NO: 744) |
| | NO: 742) | | |
| N1wc614 | GYNFTGYY | INPNSGG | ATDSFDY (SEQ ID NO: 209) |
| | (SEQ ID | (SEQ ID | |
| | NO: 207) | NO: 208) | |
| N1wc621 | GYTFTGYY | ISPNSGV (SEQ | AREGREDGVLFDY (SEQ ID NO: 747) |
| | (SEQ ID | ID NO: 746) | |
| | NO: 745) | | |
| N1wc624 | GYNFTGYY | INPNSGG | ATDSFDY (SEQ ID NO: 209) |
| | (SEQ ID | (SEQ ID | |
| | NO: 207) | NO: 208) | |
| N1wc633 | GYNFTGYY | INPNSGG | ATDSFDY (SEQ ID NO: 209) |
| | (SEQ ID | (SEQ ID | |
| | NO: 207) | NO: 208) | |
| N1wc441 | GYSFTDYY | INPNNGG | ATDGPPGTGNAFDI (SEQ ID NO: 749) |
| | (SEQ ID | (SEQ ID | |
| | NO: 748) | NO: 724) | |
| N1wc476 | GYNFTGYY | INPNSGG | ATDSFDY (SEQ ID NO: 209) |
| | (SEQ ID | (SEQ ID | |
| | NO: 207) | NO: 208) | |
| N1wc516 | GYNFTGYY | INPNSGG | ATDSFDY (SEQ ID NO: 209) |
| | (SEQ ID | (SEQ ID | |
| | NO: 207) | NO: 208) | |
| N1wc535 | GYNFTGYY | INPNSGG | ATDSFDY (SEQ ID NO: 209) |
| | (SEQ ID | (SEQ ID | |
| | NO: 207) | NO: 208) | |
| N1wc540 | GYNFTGYY | INPNSGG | ATDSFDY (SEQ ID NO: 209) |
| | (SEQ ID | (SEQ ID | |
| | NO: 207) | NO: 208) | |
| N1wc558 | GYNFTGYY | INPNSGG | ATDSFDY (SEQ ID NO: 209) |
| | (SEQ ID | (SEQ ID | |
| | NO: 207) | NO: 208) | |
| N1wc570 | GFTFTSSA | IVVGSGN | VADTVY (SEQ ID NO: 215) |
| | (SEQ ID | (SEQ ID | |
| | NO: 213) | NO: 214) | |
| N1wc572 | GYNFTGYY | INPNSGG | ATDSFDY (SEQ ID NO: 209) |
| | (SEQ ID | (SEQ ID | |
| | NO: 207) | NO: 208) | |
| N1wc586 | GYTFPDFY | INSNSGS (SEQ | ARGAYDYVWGTSRYVADAFDI (SEQ |
| | (SEQ ID | ID NO: 751) | ID NO: 752) |
| | NO: 750) | | |
| N1wc102 | GYNFTGYY | INPNSGG | ATDSFDY (SEQ ID NO: 209) |
| | (SEQ ID | (SEQ ID | |
| | NO: 207) | NO: 208) | |
| N1wc113 | GYSFSRYY | INPGGGT | ARSNYDILAGYGPDAFDF (SEQ ID |
| | (SEQ ID | (SEQ ID | NO: 272) |
| | NO: 270) | NO: 271) | |
| N1wc117 | GYNFTGYY | INPNSGG | ATDSFDY (SEQ ID NO: 209) |
| | (SEQ ID | (SEQ ID | |
| | NO: 207) | NO: 208) | |
| N1wc125 | GFTFTGYY | VDPYNGA | ARGWELDS (SEQ ID NO: 755) |
| | (SEQ ID | (SEQ ID | |
| | NO: 753) | NO: 754) | |
| N1wc127 | GYNFTGYY | INPNSGG | ATDSFDY (SEQ ID NO: 209) |
| | (SEQ ID | (SEQ ID | |
| | NO: 207) | NO: 208) | |
| N1wc129 | GYNFTGYY | IDPNSGG | ASDSFDH (SEQ ID NO: 756) |
| | (SEQ ID | (SEQ ID | |
| | NO: 207) | NO: 208) | |
| N1wc130 | GYNFTGYY | INPNSGG | ATDSFDY (SEQ ID NO: 209) |
| | (SEQ ID | (SEQ ID | |
| | NO: 207) | NO: 208) | |
| N1wc131 | GYNFTGYY | INPNSGG | ATDSFDY (SEQ ID NO: 209) |
| | (SEQ ID | (SEQ ID | |
| | NO: 207) | NO: 208) | |
| N1wc132 | GYNFTGYY | INPNSGG | VSDSFDH (SEQ ID NO: 757) |
| | (SEQ ID | (SEQ ID | |
| | NO: 207) | NO: 208) | |
| N1wc135 | GYNFTGYY | INPNSGG | ATDSFDY (SEQ ID NO: 209) |
| | (SEQ ID | (SEQ ID | |
| | NO: 207) | NO: 208) | |
| N1wc150 | GYNFTGYY | INPNSGG | ATDSFDY (SEQ ID NO: 209) |
| | (SEQ ID | (SEQ ID | |
| | NO: 207) | NO: 208) | |
| N1wc164 | GYTFTKYY | INPTNGY (SEQ | TRSYSNYDSFDI (SEQ ID NO: 760) |
| | (SEQ ID | ID NO: 759) | |
| | NO: 758) | | |
| N1wc170 | GYTFSSFG | VSPYSGD | ANSPISDGSSSSYDN (SEQ ID NO: 763) |
| | (SEQ ID | (SEQ ID | |
| | NO: 761) | NO: 762) | |
| N1wc11 | GFTFTNSA | IVVGSGN | VADTVY (SEQ ID NO: 215) |
| | (SEQ ID | (SEQ ID | |
| | NO: 726) | NO: 214) | |
| N1wc13 | GYNFTGYY | INPNSGG | ATDSFDY (SEQ ID NO: 209) |
| | (SEQ ID | (SEQ ID | |
| | NO: 207) | NO: 208) | |
| N1wc15 | GYNFTGYY | INPNSGG | ATDSFDY (SEQ ID NO: 209) |
| | (SEQ ID | (SEQ ID | |
| | NO: 207) | NO: 208) | |
| N1wc16 | GYTFTDNY | INVNSGA (SEQ | ARDFRYSGVDV (SEQ ID NO: 766) |
| | (SEQ ID | ID NO: 765) | |
| | NO: 764) | | |
| N1wc177 | GGTFSNYA | IIPGFGK (SEQ | ARDRGPDIGVAHFDS (SEQ ID NO: 768) |
| | (SEQ ID | ID NO: 767) | |
| | NO: 279) | | |
| N1wc182 | GFTFTSSA | IVVGSGN | VADTVY (SEQ ID NO: 215) |
| | (SEQ ID | (SEQ ID | |
| | NO: 213) | NO: 214) | |
| N1wc21 | GYTFTDHY | MNPKSGG | ASDPTIPPSPNYDYYYYAMDL (SEQ ID |
| | (SEQ ID | (SEQ ID | NO: 771) |
| | NO: 769) | NO: 770) | |
| N1wc24 | GGSMTSNF | VLYTGS (SEQ | ARGNGWYLP (SEQ ID NO: 774) |
| | (SEQ ID | ID NO: 773) | |
| | NO: 772) | | |
| N1wc39 | GHTFTNYA | INADNGN | ARDPWSGVLDH (SEQ ID NO: 777) |
| | (SEQ ID | (SEQ ID | |
| | NO: 775) | NO: 776) | |
| N1wc45 | GYSFRSYG | ISAYNGN (SEQ | VRGDRHYDILTGYSPVWFDP (SEQ ID |
| | (SEQ ID | ID NO: 292) | NO: 779) |
| | NO: 778) | | |
| N1wc72 | GYNFTGYY | INPNSGG | ATDSFDY (SEQ ID NO: 209) |
| | (SEQ ID | (SEQ ID | |
| | NO: 207) | NO: 208) | |
| N1wc79 | GYNFTGYY | INPNSGG | ATDSFDY (SEQ ID NO: 209) |
| | (SEQ ID | (SEQ ID | |
| | NO: 207) | NO: 208) | |
| N1wc82 | GYNFTGYY | INPNSGG | ATDSFDY (SEQ ID NO: 209) |
| | (SEQ ID | (SEQ ID | |
| | NO: 207) | NO: 208) | |
| N1wc197 | GYNFTGYY | INPNSGG | ATDSFDY (SEQ ID NO: 209) |
| | (SEQ ID | (SEQ ID | |
| | NO: 207) | NO: 208) | |
| N1wc241 | GYTFNDYY | INPHTGV (SEQ | ARERSEWRNTVSSPSEYFHH (SEQ ID |
| | (SEQ ID | ID NO: 781) | NO: 782) |
| | NO: 780) | | |
| N1wc266 | GYNFTGYY | INPNSGG | ATDSFDY (SEQ ID NO: 209) |
| | (SEQ ID | (SEQ ID | |
| | NO: 207) | NO: 208) | |
| N1wc281 | GYTFTTNS | INPRDGS | ARDGLDYRDTSRILAPTDV (SEQ ID |
| | (SEQ ID | (SEQ ID | NO: 785) |
| | NO: 783) | NO: 784) | |
| N1wc289 | GYKFTDYY | VDPTDDE | IRGIAALAD (SEQ ID NO: 788) |
| | (SEQ ID | (SEQ ID | |
| | NO: 786) | NO: 787) | |
| N1wc301 | GYIFGTYA | INAGTGN | TRGDYYDSRGYYNRFDY (SEQ ID |
| | (SEQ ID | (SEQ ID | NO: 791) |
| | NO: 789) | NO: 790) | |
| N1wc336 | GYNFTGYY | INPNSGG | ATDSFDY (SEQ ID NO: 209) |
| | (SEQ ID | (SEQ ID | |
| | NO: 207) | NO: 208) | |
| N1wc403 | GYNFTGYY | INPNSGG | ATDSFDY (SEQ ID NO: 209) |
| | (SEQ ID | (SEQ ID | |
| | NO: 207) | NO: 208) | |
| N1wc338 | GYNFTGYY | INPNSGG | ATDSFDY (SEQ ID NO: 209) |
| | (SEQ ID | (SEQ ID | |
| | NO: 207) | NO: 208) | |
| N1wc225 | GYNFTGYY | INPNSGG | ATDSFDY (SEQ ID NO: 209) |
| | (SEQ ID | (SEQ ID | |
| | NO: 207) | NO: 208) | |
| N1wc295 | GYNFTGYY | INPNSGG | ATDSFDY (SEQ ID NO: 209) |
| | (SEQ ID | (SEQ ID | |
| | NO: 207) | NO: 208) | |
| N1wc239 | GYNFTGYY | INPNSGG | ATDSFDY (SEQ ID NO: 209) |
| | (SEQ ID | (SEQ ID | |
| | NO: 207) | NO: 208) | |
| N1wc248 | GYNFTGYY | INPNSGG | ATDSFDY (SEQ ID NO: 209) |
| | (SEQ ID | (SEQ ID | |
| | NO: 207) | NO: 208) | |
| N1wc319 | GYNFTGYY | INPNSGG | ATDSFDY (SEQ ID NO: 209) |
| | (SEQ ID | (SEQ ID | |
| | NO: 207) | NO: 208) | |

Light Chain

| Clone Name | LC-CDR1 | LC-CDR2 | LC-CDR3 |
|---|---|---|---|
| hN1S3-001 | SSNIGSNT (SEQ | SN | AAWDDSLNGYV (SEQ |
| | ID NO: 226) | | ID NO: 215) |
| hN1S3-002 | SSNIGSNT (SEQ | NN | ASWDDSLNGRV (SEQ |
| | ID NO: 226) | | ID NO: 303) |
| hN1S3-003 | SSNIGSNT (SEQ | SN | AAWDDSLNGWV (SEQ |
| | ID NO: 226) | | ID NO: 792) |
| hN1S3-006 | NSNIGSNT (SEQ | SN | AAWDDSLNGYV (SEQ |
| | ID NO: 246) | | ID NO: 215) |
| hN1S3-007 | RSNIGSNA (SEQ | NT | SSWDDSLNGLE (SEQ ID |
| | ID NO: 793) | | NO: 794) |
| hN1S3-023 | SSNIGSNT (SEQ | SN | AAWDDSLNGYV (SEQ |
| | ID NO: 226) | | ID NO: 315) |
| hN1S3-030 | SSNIGSNY (SEQ | RN | GSWDDSLSGVV (SEQ |
| | ID NO: 228) | | ID NO: 795) |
| hN1S3-032 | SSNIGAGYD | GN | QSYDSSLSGSV (SEQ ID |
| | (SEQ ID NO: 241) | | NO: 248) |
| hN1S3-035 | SSNIGAGYD | GN | QSYDSSLSGSV (SEQ ID |
| | (SEQ ID NO: 241) | | NO: 248) |
| hN1S3-036 | SSNIGSNY (SEQ | RN | AAWDDSLSGVV (SEQ |
| | ID NO: 228) | | ID NO: 225) |
| hN1S3-038 | SSNIGSNY (SEQ | RN | AAWDDSLSGVV (SEQ |
| | ID NO: 228) | | ID NO: 225) |
| hN1S3-040 | SSNIGRNS (SEQ | GD | AAWDDSLNGWV (SEQ |
| | ID NO: 796) | | ID NO: 792) |
| hN1S3-053 | RSNIGAGYD | GN | QSYDSSLSGSV (SEQ ID |
| | (SEQ ID NO: 797) | | NO: 248) |
| hN1S3-055 | SSNIGSNT (SEQ | TN | AAWDDSLNGVV (SEQ |
| | ID NO: 226) | | ID NO: 223) |
| hN1S3-147 | SSNIGSNP (SEQ | SD | ATWDDSLSGWV (SEQ |
| | ID NO: 310) | | ID NO: 798) |
| hN1S3-074 | SSNIGSNT (SEQ | IS | AAWDDSLNGAV (SEQ |
| | ID NO: 226) | | ID NO: 234) |
| hN1S3-077 | HSNIGNFY (SEQ | DN | STWDRSLSASV (SEQ ID |
| | ID NO: 799) | | NO: 800) |
| hN1S3-082 | GSNIGSNT (SEQ | NN | AAWDDSLNGWL (SEQ |
| | ID NO: 801) | | ID NO: 802) |
| hN1S3-083 | SRDVGAYNY | EV | SSYAGSDSWV (SEQ ID |
| | (SEQ ID NO: 803) | | NO: 804) |
| hN1S3-087 | SGSIGSSY (SEQ | ED | QSYDANNRV (SEQ ID |
| | ID NO: 805) | | NO: 806) |
| hN1S3-088 | SSNIGNDV (SEQ | ND | AVWDDSLDGWV (SEQ |
| | ID NO: 807) | | ID NO: 808) |
| hN1S3-091 | NSNIGSNY (SEQ | SN | AAWDDSLNGHI (SEQ ID |
| | ID NO: 224) | | NO: 809) |
| hN1S3-092 | SSNIGANY (SEQ | KN | AAWDDSLSGLV (SEQ ID |
| | ID NO: 810) | | NO: 329) |
| hN1S3-095 | NSNIGSNT (SEQ | GN | ATWDDSLNGFV (SEQ ID |
| | ID NO: 246) | | NO: 811) |
| hN1S3-096 | SSNIASNS (SEQ | RN | AAWDDNLYGVV (SEQ |
| | ID NO: 812) | | ID NO: 813) |
| hN1S3-107 | SSNIGSNT (SEQ | NN | ASWDDSLNGRV (SEQ |
| | ID NO: 226) | | ID NO: 303) |
| hN1S3-112 | SSNIGNNY (SEQ | RN | GGWDDSLSGQVV (SEQ |
| | ID NO: 814) | | ID NO: 815) |
| hN1S3-124 | SSNIGSNT (SEQ | AT | AAWDDSLNGKV (SEQ |
| | ID NO: 226) | | ID NO: 816) |
| hN1S3-127 | TSNIGGNP (SEQ | RD | ASWDDSLGGVV (SEQ |
| | ID NO: 817) | | ID NO: 818) |
| hN1S3-133 | SSNIGNNA (SEQ | YD | ATWDDSLNAWV (SEQ |
| | ID NO: 222) | | ID NO: 819) |
| hN1S3-135 | SFNIGNNA (SEQ | YD | AAWDDSLNAVV (SEQ ID |
| | ID NO: 820) | | NO: 333) |
| hN1S3-136 | TSNIGDNT (SEQ | SN | AAWDDSRNGYV (SEQ |
| | ID NO: 821) | | ID NO: 822) |
| hN1S3-141 | YSNIGSNY (SEQ | TN | AAWDDSVGVLL (SEQ ID |
| | ID NO: 244) | | NO: 336) |
| hN1S3-151 | SSNIGSNT (SEQ | SN | AAWDDSLNGGV (SEQ |
| | ID NO: 226) | | ID NO: 823) |
| hN1S3-153 | SSNTGAGYY | GN | QSYDRSLSGWV (SEQ |
| | (SEQ ID NO: 824) | | ID NO: 825) |
| hN1S3-177 | SSNIGTNY (SEQ | SN | AAWDDSLSGLV (SEQ ID |
| | ID NO: 826) | | NO: 329) |
| hN1S3-181 | SPNIGSNT (SEQ | TN | AAWDDSLLGVV (SEQ ID |
| | ID NO: 235) | | NO: 236) |
| hN1S3-182 | SSNIGSPYD | GN | QTYDSTLNGAV (SEQ ID |
| | (SEQ ID NO: 827) | | NO: 828) |
| hN1S3-184 | SSNIGGNY (SEQ | RS | AAWDDSLHGVI (SEQ ID |
| | ID NO: 829) | | NO: 830) |
| hN1S3-190 | SLRSNY (SEQ ID | SE | HSRGSSGNHKV (SEQ |
| | NO: 831) | | ID NO: 832) |
| hN1S3-191 | SSNIGSNT (SEQ | TN | AAWDDSLNGYV (SEQ |
| | ID NO: 235) | | ID NO: 215) |
| hN1S3-108 | QGISSW (SEQ ID | KA | QQSYSTPWT (SEQ ID |
| | NO: 833) | | NO: 834) |
| hN1S3-130 | QGISSW (SEQ ID | KA | QQSYSTPWT (SEQ ID |
| | NO: 833) | | NO: 834) |
| N1wc614 | SSNIGSNT (SEQ | SN | AAWDDSLNGLV (SEQ ID |
| | ID NO: 235) | | NO: 233) |
| N1wc621 | SSNLGAGYD | NN | QTYDTRLSGSML (SEQ |
| | (SEQ ID NO: 835) | | ID NO: 836) |
| N1wc624 | SSNIGSNT (SEQ | SN | AAWDDSLNGKV (SEQ |
| | ID NO: 235) | | ID NO: 816) |
| N1wc633 | SSNIGSYT (SEQ | SN | AAWDDSLSDVL (SEQ ID |
| | ID NO: 837) | | NO: 838) |
| N1wc441 | ALPDKY (SEQ ID | DD | YSTDSSGNERV (SEQ ID |
| | NO: 326) | | NO: 327) |
| N1wc476 | NSNIGNNY (SEQ | RN | ASWDDSLSGLV (SEQ ID |
| | ID NO: 839) | | NO: 840) |
| N1wc516 | SSNIGSNT (SEQ | SN | AAWDDSLNDVV (SEQ |
| | ID NO: 235) | | ID NO: 841) |
| N1wc535 | SSNIGSNT (SEQ | SD | AGWDDSLNAYL (SEQ ID |
| | ID NO: 235) | | NO: 842) |
| N1wc540 | SSNIGSYT (SEQ | SN | AAWDDSLSDVL (SEQ ID |
| | ID NO: 837) | | NO: 838) |
| N1wc558 | SSNIGSNS (SEQ | SN | AAWDDSLNNLV (SEQ ID |
| | ID NO: 328) | | NO: 843) |
| N1wc570 | TSNIGNNN (SEQ | SN | ATWDDSLSAWV (SEQ |
| | ID NO: 844) | | ID NO: 845) |
| N1wc572 | SSNIGSNP (SEQ | SD | AAWDDSLNAVV (SEQ ID |
| | ID NO: 310) | | NO: 333) |
| N1wc586 | QGISSW (SEQ ID | KA | QQSYSTPWT (SEQ ID |
| | NO: 833) | | NO: 834) |
| N1wc102 | SSNIGSNP (SEQ | GD | AAWDDSLNGVV (SEQ |
| | ID NO: 310) | | ID NO: 223) |
| N1wc113 | SSNIGAGND | GN | QSYDSSLGGSDV (SEQ |
| | (SEQ ID NO: 846) | | ID NO: 847) |
| N1wc117 | SSNIGGNT (SEQ | GT | AAWDDFLNGKV (SEQ |
| | ID NO: 848) | | ID NO: 849) |
| N1wc125 | SSDVGGYNY | EG | SSYTTRSTRV (SEQ ID |
| | (SEQ ID NO: 266) | | NO: 267) |
| N1wc127 | SSNIGSNG (SEQ | SN | AAWDDSLNDYV (SEQ |
| | ID NO: 850) | | ID NO: 851) |
| N1wc129 | GSNIGRNA (SEQ | SN | ATWDDSLNDYV (SEQ ID |
| | ID NO: 852) | | NO: 853) |
| N1wc130 | GSNIGAGYD | AN | QSYDSSLSGSV (SEQ ID |
| | (SEQ ID NO: 854) | | NO: 248) |
| N1wc131 | SSNIGINY (SEQ | YN | AAWDDSLSGLV (SEQ ID |
| | ID NO: 855) | | NO: 329) |
| N1wc132 | SSNIGSNP (SEQ | SD | AAWDDSQNAVV (SEQ |
| | ID NO: 310) | | ID NO: 856) |
| N1wc135 | SSNIGSNY (SEQ | RN | AAWDDSLSGVV (SEQ |
| | ID NO: 228) | | ID NO: 225) |
| N1wc150 | GSNIGKNT (SEQ | SN | AAWDDSLNDMV (SEQ |
| | ID NO: 857) | | ID NO: 858) |
| N1wc164 | SSNIGAGYD | RN | QSYDTRLSDSVI (SEQ |
| | (SEQ ID NO: 241) | | ID NO: 859) |
| N1wc170 | NIGTKS (SEQ ID | ED | YSADNTGDQRM (SEQ |
| | NO: 860) | | ID NO: 861) |
| N1wc11 | SSNLGINT (SEQ | SD | AAWDDSLNGWV (SEQ |
| | ID NO: 862) | | ID NO: 792) |
| N1wc13 | SSNIGSNY (SEQ | RN | AAWDDSLSGLV (SEQ ID |
| | ID NO: 228) | | NO: 329) |
| N1wc15 | SSSIGSNT (SEQ | SN | AAWDDSLNGLV (SEQ ID |
| | ID NO: 863) | | NO: 233) |
| N1wc16 | AIERKS (SEQ ID | DD | QVWDTSSGHDAVV |
| | NO: 864) | | (SEQ ID NO: 865) |
| N1wc177 | SSNIGKYI (SEQ | TY | ASWDDSLNVVV (SEQ ID |
| | ID NO: 866) | | NO: 867) |
| N1wc182 | SSNIGSNT (SEQ | SN | AAWDDSLNGNWV (SEQ |
| | ID NO: 235) | | ID NO: 868) |
| N1wc21 | ALPDKY (SEQ ID | DD | YSTDSSGNERV (SEQ ID |
| | NO: 326) | | NO: 327) |
| N1wc24 | SSNIENNI (SEQ | YD | AAWDDSLKAVV (SEQ ID |
| | ID NO: 869) | | NO: 870) |
| N1wc39 | QNINKW (SEQ ID | TA | QQAFSFPIT (SEQ ID |
| | NO: 871) | | NO: 872) |
| N1wc45 | SLRSYY (SEQ ID | GK | NSRDSSGNHVV (SEQ |
| | NO: 873) | | ID NO: 874) |
| N1wc72 | YSNIGSNY (SEQ | TN | AAWDDSVGVLL (SEQ ID |
| | ID NO: 244) | | NO: 336) |
| N1wc79 | TSNIGKNT (SEQ | SD | AAWDDSLDGLV (SEQ ID |
| | ID NO: 875) | | NO: 876) |
| N1wc82 | NSNIGNNY (SEQ | DS | ATWDAGLSVLI (SEQ ID |
| | ID NO: 839) | | NO: 877) |
| N1wc197 | SSNIGSNY (SEQ | TN | AAWDDSLSGVV (SEQ |
| | ID NO: 228) | | ID NO: 225) |
| N1wc241 | FSNIGSNN (SEQ | RN | AVRDDSLSGHYV (SEQ |
| | ID NO: 878) | | ID NO: 879) |
| N1wc266 | RSNIGSNA (SEQ | NT | SSWDDSLNGLE (SEQ ID |
| | ID NO: 793) | | NO: 794) |
| N1wc281 | KLGDKY (SEQ ID | QD | QAWDSNTDHVI (SEQ ID |
| | NO: 880) | | NO: 881) |
| N1wc289 | SDINLETSR (SEQ | YQSDSD | MIWHSGGSV (SEQ ID |
| | ID NO: 882) | (SEQ ID | NO: 884) |
| | | NO: 883) | |
| N1wc301 | SSNIGKNN (SEQ | RN | AARDDSLNGYV (SEQ ID |
| | ID NO: 885) | | NO: 886) |
| N1wc336 | YSNIGSNY (SEQ | TN | AAWDDSVSVLL (SEQ ID |
| | ID NO: 244) | | NO: 245) |
| N1wc403 | SSNIGSNT (SEQ | TN | AAWDDSLNGYV (SEQ |
| | ID NO: 226) | | ID NO: 215) |
| N1wc338 | SSNIGTYT (SEQ | SN | AAWDDSLHGYV (SEQ |
| | ID NO: 887) | | ID NO: 888) |
| N1wc225 | SSNIGAGYD | GN | QSYDSSLSGSV (SEQ ID |
| | (SEQ ID NO: 241) | | NO: 248) |
| N1wc295 | RSNIGSNT (SEQ | SN | AAWDDNLNGYV (SEQ |
| | ID NO: 889) | | ID NO: 890) |
| N1wc239 | HSNIRSND (SEQ | SN | ATWDDSLSGVV (SEQ ID |
| | ID NO: 891) | | NO: 892) |
| N1wc248 | SSNIGSNS (SEQ | RN | ATWDDSLSGVV (SEQ ID |
| | ID NO: 329) | | NO: 892) |
| N1wc319 | SSNIGSNS (SEQ | SD | ATWDDSLTGLV (SEQ ID |
| | ID NO: 328) | | NO: 893) |

APPENDIX V
Constant Region DNA Sequences

>Lambda Light Chain Constant Region
CAGCCCAAGGCCAACCCCACCGTGACCCTGTTCCCCCCATCTTCTGAGGAGCTGCAAGCCAACAAGGCCACCCTGGT
GTGCCTGATCTCTGACTTCTACCCTGGCGCTGTGACAGTGGCCTGGAAGGCTGATGGCTCTCCTGTGAAGGCTGGCG
TGGAGACCACCAAGCCATCTAAGCAGTCTAACAACAAGTATGCTGCCTCTTCTTACCTGTCTCTGACCCCTGAGCAG
TGGAAGAGCCACCGGTCTTACTCTTGCCAGGTGACCCATGAGGGCTCTACAGTGGAGAAGACAGTGGCCCCCACAGA
GTGCTCTTGA (SEQ ID NO: 894)
>Kappa Light Chain Constant Region
CGTACGGTGGCTGCACCATCTGTCTTCATCTTCCCGCCATCTGATGAGCAGTTGAAATCTGGAACTGCCTCTGTTGT
GTGCCTGCTGAATAACTTCTATCCCAGAGAGGCCAAAGTACAGTGGAAGGTGGATAACGCCCTCCAATCGGGTAACT
CCCAGGAGAGTGTCACAGAGCAGGACAGCAAGGACAGCACCTACAGCCTCAGCAGCACCCTGACGCTGAGCAAAGCA
GACTACGAGAAACACAAAGTCTACGCCTGCGAAGTCACCCATCAGGGCCTGAGCTCGCCCGTCACAAAGAGCTTCAA
CAGGGGAGAGTGTTAG (SEQ ID NO: 895)
>IgG1 Heavy Chain Constant Region
GCATCCACCAAGGGCCCATCTGTCTTCCCCCTGGCCCCATCCTCCAAGAGCACCTCTGGCGGCACAGCTGCCCTGGG
CTGCCTGGTGAAGGACTACTTCCCTGAGCCTGTGACAGTGTCCTGGAACTCTGGCGCCCTGACCAGCGGCGTGCACA
CCTTCCCTGCTGTGCTCCAGTCCTCTGGCCTGTACTCCCTGAGCAGCGTGGTGACAGTGCCATCCAGCAGCCTGGGC
ACCCAGACCTACATCTGCAATGTGAACCACAAGCCCAGCAACACCAAGGTGGACAAGCGGGTGGAGCCCAAGTCCTG
TGACAAGACCCACACCTGCCCCCCATGCCCCGCCCCTGAGCTGCTGGGCGGCCCATCTGTCTTCCTGTTCCCCCCCA
AGCCCAAGGACACCCTGATGATCTCCCGGACCCCCGAGGTGACCTGTGTGGTGGTGGATGTGAGCCATGAGGACCCC
GAGGTGAAGTTCAACTGGTATGTGGATGGCGTGGAGGTGCACAACGCCAAGACCAAGCCCCGGGAGGAGCAGTACAA
CAGCACCTACCGGGTGGTGAGCGTGCTGACAGTGCTGCATCAGGACTGGCTGAATGGCAAGGAGTACAAGTGCAAGG
TGTCCAACAAGGCCCTGCCTGCCCCCATTGAGAAGACCATCTCCAAGGCCAAGGGCCAGCCCCGGGAGCCCCAGGTC
TACACCCTGCCCCCCTCCCGGGAGGAGATGACCAAGAACCAGGTGAGCCTGACCTGCCTGGTGAAGGGCTTCTACCC
CAGCGACATTGCTGTGGAGTGGGAGAGCAACGGCCAGCCTGAGAACAACTACAAGACCACCCCCCCTGTGCTGGACT
CTGATGGCTCCTTCTTCCTGTACAGCAAGCTGACAGTGGACAAGAGCCGGTGGCAGCAGGGCAATGTCTTCTCCTGC
TCTGTGATGCATGAGGCCCTGCACAACCACTACACCCAGAAGAGCCTGTCCCTGTCCCCCGGCAAGTGA
(SEQ ID NO: 896)

APPENDIX VI
Constant Region Protein Sequences

>Lambda Light Chain Constant Region
QPKANPTVTLFPPSSEELQANKATLVCLISDFYPGAVTVAWKADGSPVKAGVETTKPSKQSNNKYAASSYLSLTPEQ
WKSHRSYSCQVTHEGSTVEKTVAPTECS (SEQ ID NO: 897)
>Kappa Light Chain Constant Region
RTVAAPSVFIFPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGEC (SEQ ID NO: 898)
>IgG1 Heavy Chain Constant Region
ASTKGPSVFPLAPSSKSTSGGTAALGCLVKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYSKLTVDKSRWQQGNVFSC
SVMHEALHNHYTQKSLSLSPGK (SEQ ID NO: 899)

APPENDIX VII
Leader DNA sequences

>Light Chain Leader Sequence
ATGAGTGTGCCCACTCAGGTCCTGGGGTTGCTGCTGCTGTGGCTTACA
GATGCCAGATGC (SEQ ID NO: 900)
>Heavy Chain Leader Sequence
ATGGAATGGAGCTGGGTCTTTCTCTTCTTCCTGTCAGTAACTACAGGTGT
CCACTCG (SEQ ID NO: 901)

APPENDIX VIII
Leader Protein Sequences

>Light Chain Leader Sequence
MSVPTQVLGLLLLWLTDARC (SEQ ID NO: 902)
>Heavy Chain Leader Sequence
MEWSWVFLFFLSVTTGVHS (SEQ ID NO: 903)

APPENDIX IX
Group I - Notch1 specific antibodies
A. Aminoacid sequence
A.1-Binding to NRR

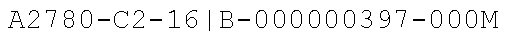

Heavy chain
EVQLVQSGAEVKKPGSSVKVSCKASGGTFSSFAFSWVRQAPGQGLDWMGRIIPILGRTN
YAQKFQGRVTFTADTSTSTVYMELSSLSSEDTAVYYCARDREYSLSLGGSDYWGQGTL
VTVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKRVEPKSCDKTHTCPPCPAP
ELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGK (SEQ ID NO: 904)
Light chain
QAVLTQPSSVSGAPGQRVTISCIGSSSNIGAGYDVHWYRQPPGTAPQLLIYANTNRPSGV
PDRFSGSRSGTSASLAITGIQAEDEADYYCQSYDSSLSGRVFGGGTKVTVLGQPKAAPSV
TLFPPSSEELQANKATLVCLISDFYPGAVTVAWKADSSPVKAGVETTTPSKQSNNKYAA
SSYLSLTPEQWKSHRSYSCQVTHEGSTVEKTVAPTECS (SEQ ID NO: 905)

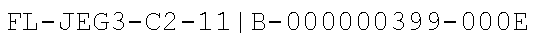

Heavy chain
EVQLQLVETGGGLVQPVGSLRLSCAASGFSFDNYAMSWVRQAPGKGLEWVSSISGSGG
QIFYADSVKGRFTISRDNAKNTLVLQMNSLRVDDTAVYYCARDFYRTPGYNTNWGYW
GRGTMVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRVEPKSCDKTHTC
PPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDSDG
SFFLYSKLTVDKSRWQQGNVFSCSVMHEALHNHYTQKSLSLSPGK (SEQ ID NO: 906)
Light chain
SYVLTQPPSVSVSPGETASISCSGDGLGNKYVAWYQQKPGQSPVLVIQQDSKRPSGIPER
FSGSNSGDTATLTISGTQSLDEGDYYCQAWDRPAVIFGGGTKLTVLGQPKAAPSVTLFPP
SSEELQANKATLVCLISDFYPGAVTVAWKADSSPVKAGVETTTPSKQSNNKYAASSYLS
LTPEQWKSHRSYSCQVTHEGSTVEKTVAPTECS (SEQ ID NO: 907)
A.2-Additional Notch Antibodies

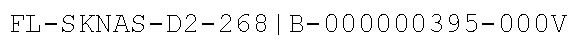

Heavy chain
EVQLLESGGGLVQPGGSLRLSCAASGFTFSSYAMSWVRQAPGKGLEWVSAISGSGGSTY
YADSVKGRFTISRDNSKNTLYLQMNSLRAEDTAVYYCARHGHSSGYFVYWGKGTMVT
VSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKRVEPKSCDKTHTCPPCPAPEL
LGGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGK (SEQ ID NO: 908)
Light chain
QAVLTQPSSVSGAPGQRVTISCTGTSSNIGAPYDVNWYQQLPGIAPKLLISGNTNRPSGV
PDRFSGSKSGASASLAITGLQADDEADYYCQSYDHSLSGWVFGGGTKVTVLGQPKAAP
SVTLFPPSSEELQANKATLVCLISDFYPGAVTVAWKADSSPVKAGVETTTPSKQSNNKY
AASSYLSLTPEQWKSHRSYSCQVTHEGSTVEKTVAPTECS (SEQ ID NO: 909)

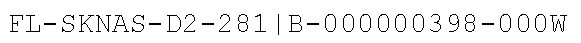

Heavy chain
EVQLWESGGGLVQPGGSLRLSCAASGFTFSSYAMSWVRQAPGKGLEWVSAISGSGGST
YYADSVKGRFTISRDNSKNTLYLQMNSLRAEDTAVYYCARSQLPYCGGDCYGSDYWG
RGTMVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEPKSCDKTHTCP
PCPAPELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK (SEQ ID NO: 910)
Light chain
QAVLTQPSSVSVSPGQTASITCSGDKLGDKYASWYQQRPGQSPLLVIYKDSERPSGIPERFSGSSAGNT
ATLTISGTQPMDEADYYCQAWDSGAVFGGGTKVTVLGQPKAAPSVTLFPPSSEELQANKATLVCLISDFY
PGAVTVAWKADSSPVKAGVETTTPSKQSNNKYAASSYLSLTPEQWKSHRSYSCQVTHEGSTVEKTVAP
TECS (SEQ ID NO: 911)

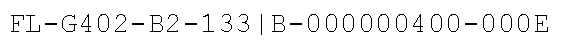

Heavy chain
QVQLVQSGAEVKKPGESLRISCKASGYTFTDYYLHWVRQAPGKGLEWMGWLNPNSGG
TNYAQKFQGRVTMTRDTSISTAYMELSRLRSDDTAVYFCARGAYDYVWGTSRYVADA
FDIWGQGTMVTVSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDKRVEPKSCDK
THTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK (SEQ ID NO: 912)
Light chain
QSVLTQPPSASGTPGQRVTISCSGSSSNIGSNTVNWYQRLPGAAPQLLIYNNDQRPSGIPD
RFSGSKSGTSGSLVISGLQSEDEADYYCASWDDSLNGRVFGGGTKLTVLGQPKAAPSVT
LFPPSSEELQANKATLVCLISDFYPGAVTVAWKADSSPVKAGVETTTPSKQSNNKYAAS
SYLSLTPEQWKSHRSYSCQVTHEGSTVEKTVAPTECS (SEQ ID NO: 913)

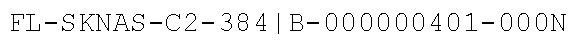

Heavy chain
EVQLVQSGAEVKKPGSSVKVSCKASGGTLSSSAISWVRQAPGEGLEWMGGIIPMFDSTN
YAQKFQGRVTITADESTSTAFMELSSLRSDDTAVYYCGRDPYYDRLSGGGYMDVWGQ
GTLVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDKTHTCPPC
PAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK (SEQ ID NO: 914)
Light chain
SYVLTQPPSVSGALRQTATVTCTGNSSNVGNQGAAWLRQHQGRPPKLLSYRNNDRPSG
VSQRFSASTSGNTASLTITGLQSEDEADYFCSAWDSSRSGWVFGGGTKLTVLGQPKAAP
SVTLFPPSSEELQANKATLVCLISDFYPGAVTVAWKADSSPVKAGVETTTPSKQSNNKY
AASSYLSLTPEQWKSHRSYSCQVTHEGSTVEKTVAPTECS (SEQ ID NO: 915)

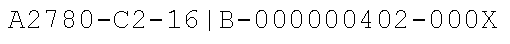

Heavy chain
EVQLVQSGAEVKEPGESLRISCKASGYDFSNYWIAWVRQMPGKGLEWMGIIYPGDSDT
RYSPSFQGQVTISVDRSISTAYLQWSSLKASDTALYYCSRPTYWSTTWGREFDYWGQGT
LVTVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKRVEPKSCDKTHTCPPCPA
PELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGK (SEQ ID NO: 916)
Light chain
SYELTQPPSVSVSPGQTATITCSGDALPKKYAYWYQQKSGQAPVLVVYEDSKRPSGIPE
RFSGSSSGTMATLTISGAQVEDEADYYCYSTDSSGNHWVFGGGTKLTVLGQPKAAPSV
TLFPPSSEELQANKATLVCLISDFYPGAVTVAWKADSSPVKAGVETTTPSKQSNNKYAA
SSYLSLTPEQWKSHRSYSCQVTHEGSTVEKTVAPTECS (SEQ ID NO: 917)

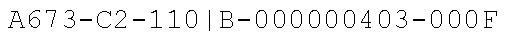

Heavy chain
QVQLVQSGTEVKKPGASVRVSCKPSADTFTSYYVHWVRQAPGQGLEWMGTINPTGTY
TRYAQQFQGRVTVTRDTSTSSVYMELSSLRSEDSAVYYCARDVRPYTAMYSFDNWGR
GTTVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDKTHTCPPC
PAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK (SEQ ID NO: 918)
Light chain
SYVLTQPPSVSVSPGQTATITCSGDALPKQYANWYQQKPGQAPVLMIYKDSDRPSGIPE
RFSGSSSGTKATLTISGVQAEDEADYYCQSADNSGTNYVFGTGTKLTVLGQPKAAPSVT
LFPPSSEELQANKATLVCLISDFYPGAVTVAWKADSSPVKAGVETTTPSKQSNNKYAAS
SYLSLTPEQWKSHRSYSCQVTHEGSTVEKTVAPTECS (SEQ ID NO: 919)
B. DNA sequence
B.1-Binding to NRR
A2780-C2-16|B-000000397-000M
Heavy chain
GTGCAGCTGGTGCAGTCTGGGGCTGAGGTGAAGAAGCCTGGGTCCTCGGTGAAGGT
CTCCTGCAAGGCTTCTGGAGGCACCTTCAGCAGCTTTGCTTTCAGCTGGGTGCGACA
GGCCCCTGGACAAGGGCTTGACTGGATGGGAAGGATCATCCCTATACTTGGTAGAA
CAAACTACGCACAGAAATTCCAGGGCAGAGTCACCTTTACCGCGGACACATCCACG
AGCACAGTCTACATGGAATTGAGCAGTCTGAGCTCTGAGGACACGGCCGTGTATTA
CTGTGCGAGAGACCGGGAATATAGCTTGAGTCTGGGGGGCAGTGACTACTGGGGCC
AGGGCACCCTGGTCACCGTCTCGAGTGCCTCCACCAAGGGCCCATCGGTCTTCCCCC
TGGCACCCTCCTCCAAGAGCACCTCTGGGGGCACAGCGGCCCTGGGCTGCCTGGTCA
AGGACTACTTCCCCGAACCGGTGACGGTGTCGTGGAACTCAGGCGCCCTGACAGCG
GCGTGCACACCTTCCCGGCTGTCCTACAGTCCTCAGGACTCTACTCCCTCAGCAGCG
TGGTGACAGTGCCCTCCAGCAGCTTGGGCACCCAGACCTACATCTGCAACGTGAATC
ACAAGCCCAGCAACACCAAGGTGGACAAGAGAGTTGAGCCCAAATCTTGTGACAAA
ACTCACACATGCCCACCGTGCCCAGCACCTGAACTCCTGGGGGGACCGTCAGTCTTC
CTCTTCCCCCCAAAACCCAAGGACACCCTCATGATCTCCCGGACCCCTGAGGTCACA
TGCGTGGTGGTGGACGTGAGCCACGAAGACCCTGAGGTCAAGTTCAACTGGTACGT
GGACGGCGTGGAGGTGCATAATGCCAAGACAAAGCCGCGGGAGGAGCAGTACAAC
AGCACGTACCGTGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAAC
CATCTCCAAAGCCAAAGGGCAGCCCCGAGAACCACAGGTGTACACCCTGCCCCCAT
CCCGGGAGGAGATGACCAAGAACCAGGTCAGCCTGACCTGCCTGGTCAAAGGCTTC
TATCCCAGCGACATCGCCGTGGAGTGGGAGAGCAATGGGCAGCCGGAGAACAACTA
CAAGACCACGCCTCCCGTGCTGGACTCCGACGGCTCCTTCTTCCTCTATAGCAAGCT
CACCGTGGACAAGAGCAGGTGGCAGCAGGGGAACGTCTTCTCATGCTCCGTGATGC
ATGAGGCTCTGCACAACCACTACACGCAGAAGAGCCTCTCCCTGTCTCCGGGTAAAT
GA (SEQ ID NO: 920)
Light chain (lambda)
CAGGCTGTGCTGACTCAGCCGTCCTCAGTGTCTGGGGCCCCAGGGCAGAGGGTCAC
CATCTCCTGCATTGGGAGCAGCTCCAACATCGGGGCGGGTTATGATGTACACTGGTA
TCGCCAGCCTCCAGGAACAGCCCCCCAACTCCTCATCTATGCAAACACCAATCGGCC
CTCTGGGGTCCCTGACCGATTCTCTGGCTCCCGGTCTGGCACCTCAGCCTCCCTGGCC
ATCACTGGGATCCAGGCTGAGGATGAGGCTGATTATTACTGCCAGTCCTACGACAGC
AGCCTGAGTGGCAGGGTGTTCGGCGGAGGGACCAAGGTCACCGTCCTAGGTGGTCA
GCCCAAGGCTGCCCCCTCGGTCACTCTGTTCCCGCCCTCCTCTGAGGAGCTTCAAGC
CAACAAGGCCACACTGGTGTGTCTCATAAGTGACTTCTACCCGGGAGCCGTGACAGT
GGCCTGGAAGGCAGATAGCAGCCCCGTCAAGGCGGGAGTGGAGACCACCACACCCT
CCAAACAAAGCAACAACAAGTACGCGGCCAGCAGCTATCTGAGCCTGACGCCTGAG
CAGTGGAAGTCCCACAGAAGCTACAGCTGCCAGGTCACGCATGAAGGGAGCACCGT
GGAGAAGACAGTGGCCCCTACAGAATGTTCATAG (SEQ ID NO: 921)
FL-JEG3-C2-11|B-000000399-000E
Heavy chain
GAGGTGCAGCTGCAGCTGGTGGAGACTGGGGGAGGCTTGGTGCAGCCTGTGGGGTC
CCTGAGACTCTCCTGTGCAGCCTCTGGATTCTCCTTTGACAACTATGCCATGAGCTG
GGTCCGCCAGGCTCCAGGGAAGGGACTGGAGTGGGTCTCAAGTATTAGTGGGAGTG
GTGGGCAAATATTCTACGCAGACTCCGTGAAGGGCCGGTTCACCATCTCCAGAGAC
AACGCCAAGAACACGCTGGTTTTGCAAATGAACAGCCTGAGAGTCGACGACACGGC
CGTCTATTACTGTGCGAGAGATTTCTACCGGACCCCCGGTTATAATACCAACTGGGG
ATACTGGGGCAGGGGGACAATGGTCACCGTCTCGAGTGCCTCCACCAAGGGCCCAT
CGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGC
GCCCTGACAGCGGCGTGCACACCTTCCCGGCTGTCCTACAGTCCTCAGGACTCTACT
CCCTCAGCAGCGTGGTGACAGTGCCCTCCAGCAGCTTGGGCACCCAGACCTACATCT
GCAACGTGAATCACAAGCCCAGCAACACCAAGGTGGACAAGAGAGTTGAGCCCAA
ATCTTGTGACAAAACTCACACATGCCCACCGTGCCCAGCACCTGAACTCCTGGGGGG
ACCGTCAGTCTTCCTCTTCCCCCCAAAACCCAAGGACACCCTCATGATCTCCCGGAC
CCCTGAGGTCACATGCGTGGTGGTGGACGTGAGCCACGAAGACCCTGAGGTCAAGT
TCAACTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCAGGA
CTGGCTGAATGGCAAGGAGTACAAGTGCAAGGTCTCCAACAAAGCCCTCCCAGCCC
CCATCGAGAAAACCATCTCCAAAGCCAAAGGGCAGCCCCGAGAACCACAGGTGTAC
ACCCTGCCCCCATCCCGGGAGGAGATGACCAAGAACCAGGTCAGCCTGACCTGCCT
GGTCAAAGGCTTCTATCCCAGCGACATCGCCGTGGAGTGGGAGAGCAATGGGCAGC
CGGAGAACAACTACAAGACCACGCCTCCCGTGCTGGACTCCGACGGCTCCTTCTTCC
TCTATAGCAAGCTCACCGTGGACAAGAGCAGGTGGCAGCAGGGGAACGTCTTCTCA
TGCTCCGTGATGCATGAGGCTCTGCACAACCACTACACGCAGAAGAGCCTCTCCCTG
TCTCCGGGTAAATGA (SEQ ID NO: 922)
Light chain
TCCTATGTGCTGACTCAGCCACCCTCAGTGTCCGTGTCCCCAGGAGAGACAGCCAGC
ATCTCCTGCTCTGGTGATGGATTGGGGAATAAATATGTTGCTTGGTATCAGCAGAAG
CCAGGCCAGTCCCCTGTCCTGGTCATCCAGCAAGATTCCAAGCGGCCCTCAGGAATC
CCTGAGCGATTCTCTGGCTCCAATTCTGGGGACACAGCCACTCTGACCATCAGCGGG
ACGCAGTCTCTGGATGAGGGTGACTATTATTGTCAGGCGTGGGACAGACCCGCTGTG
ATATTCGGCGGGGGGACCAAGCTGACCGTCCTAGGTGGTCAGCCCAAGGCTGCCCC
CTCGGTCACTCTGTTCCCGCCCTCCTCTGAGGAGCTTCAAGCCAACAAGGCCACACT
GGTGTGTCTCATAAGTGACTTCTACCCGGGAGCCGTGACAGTGGCCTGGAAGGCAG
ATAGCAGCCCCGTCAAGGCGGGAGTGGAGACCACCACACCCTCCAAACAAAGCAAC
AACAAGTACGCGGCCAGCAGCTATCTGAGCCTGACGCCTGAGCAGTGGAAGTCCCA
CAGAAGCTACAGCTGCCAGGTCACGCATGAAGGGAGCACCGTGGAGAAGACAGTG
GCCCCTACAGAATGTTCATAG (SEQ ID NO: 923)
B.2-Additional Notch Antibodies
FL-SKNAS-D2-268|B-000000395-000V
Heavy chain
GAGGTGCAGCTGTTGGAGTCTGGGGGAGGCTTGGTACAGCCTGGGGGGTCCCTGAG
ACTCTCCTGTGCAGCCTCTGGATTCACCTTTAGCAGCTATGCCATGAGCTGGGTCCG
CCAGGCTCCAGGGAAGGGGCTGGAGTGGGTCTCAGCTATTAGTGGTAGTGGTGGTA
GCACATACTACGCAGACTCCGTGAAGGGCCGGTTCACCATCTCCAGAGACAATTCC
AAGAACACGCTGTATCTGCAAATGAACAGCCTGAGAGCCGAGGACACGGCCGTGTA
TTACTGTGCGAGACATGGCCATAGCAGTGGCTACTTTGTCTATTGGGGCAAGGGGAC
AATGGTCACCGTCTCGAGTGCCTCCACCAAGGGCCCATCGGTCTTCCCCCTGGCACC
CTCCTCCAAGAGCACCTCTGGGGGCACAGCGGCCCTGGGCTGCCTGGTCAAGGACT
ACTTCCCCGAACCGGTGACGGTGTCGTGGAACTCAGGCGCCCTGACAGCGGCGTGC
ACACCTTCCCGGCTGTCCTACAGTCCTCAGGACTCTACTCCCTCAGCAGCGTGGTGA
CAGTGCCCTCCAGCAGCTTGGGCACCCAGACCTACATCTGCAACGTGAATCACAAG
CCCAGCAACACCAAGGTGGACAAGAGAGTTGAGCCCAAATCTTGTGACAAAACTCA
CACATGCCCACCGTGCCCAGCACCTGAACTCCTGGGGGGACCGTCAGTCTTCCTCTT
CCCCCCAAAACCCAAGGACACCCTCATGATCTCCCGGACCCCTGAGGTCACATGCGT
GGTGGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAGCAC
GTACCGTGTGGTCAGCGTCCTCACCGTCCTGCACCAGGACTGGCTGAATGGCAAGG
AGTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCCCG
GGAGGAGATGACCAAGAACCAGGTCAGCCTGACCTGCCTGGTCAAAGGCTTCTATC
CCAGCGACATCGCCGTGGAGTGGGAGAGCAATGGGCAGCCGGAGAACAACTACAA
GACCACGCCTCCCGTGCTGGACTCCGACGGCTCCTTCTTCCTCTATAGCAAGCTCAC
CGTGGACAAGAGCAGGTGGCAGCAGGGGAACGTCTTCTCATGCTCCGTGATGCATG
AGGCTCTGCACAACCACTACACGCAGAAGAGCCTCTCCCTGTCTCCGGGTAAATGA
(SEQ ID NO: 924)
Light chain
CAGGCTGTGCTGACTCAGCCGTCCTCAGTGTCTGGGGCCCCAGGGCAGAGGGTCAC
CATCTCCTGCACTGGGACCTCCTCCAACATCGGGGCACCTTATGATGTAAACTGGTA
CCAACAGCTGCCAGGAATAGCCCCCAAACTCCTCATCTCTGGTAACACCAATCGGCC
CTCAGGAGTCCCTGACCGATTCTCTGGCTCCAAGTCTGGCGCCTCAGCCTCCCTGGC
CATCACTGGGCTCCAGGCTGACGATGAGGCTGATTATTACTGTCAGTCCTATGACCA
CAGCCTGAGTGGTTGGGTGTTCGGCGGCGGGACCAAGGTCACCGTCCTAGGTGGTC
AGCCCAAGGCTGCCCCCTCGGTCACTCTGTTCCCGCCCTCCTCTGAGGAGCTTCAAG
CCAACAAGGCCACACTGGTGTGTCTCATAAGTGACTTCTACCCGGGAGCCGTGACA
GTGGCCTGGAAGGCAGATAGCAGCCCCGTCAAGGCGGGAGTGGAGACCACCACACC
CTCCAAACAAAGCAACAACAAGTACGCGGCCAGCAGCTATCTGAGCCTGACGCCTG
AGCAGTGGAAGTCCCACAGAAGCTACAGCTGCCAGGTCACGCATGAAGGGAGCACC
GTGGAGAAGACAGTGGCCCCTACAGAATGTTCATAG (SEQ ID NO: 925)
FL-SKNAS-D2-281|B-000000398-000W
Heavy chain
GAGGTGCAGCTGTGGGAGTCTGGGGGAGGCTTGGTACAGCCTGGGGGGTCCCTGAG
ACTCTCCTGTGCAGCCTCTGGATTCACCTTTAGCAGCTATGCCATGAGCTGGGTCCG
CCAGGCTCCAGGGAAGGGGCTGGAGTGGGTCTCAGCTATTAGTGGTAGTGGTGGTA
GCACATACTACGCAGACTCCGTGAAGGGCCGGTTCACCATCTCCAGAGACAATTCC
AAGAACACGCTGTATCTGCAAATGAACAGCCTGAGAGCCGAGGACACGGCCGTGTA
TTACTGTGCGAGAAGTCAGCTCCCATACTGTGGTGGTGACTGCTATGGTAGCGACTA
CTGGGGCCGGGGGACAATGGTCACCGTCTCGAGTGCCTCCACCAAGGGCCCATCGG
TCTTCCCCCTGGCACCCTCCTCCAAGAGCACCTCTGGGGGCACAGCGGCCCTGGGCT
GCCTGGTCAAGGACTACTTCCCCGAACCGGTGACGGTGTCGTGGAACTCAGGCGCC
CTGACAGCGGCGTGCACACCTTCCCGGCTGTCCTACAGTCCTCAGGACTCTACTCCC
TCAGCAGCGTGGTGACAGTGCCCTCCAGCAGCTTGGGCACCCAGACCTACATCTGCA
ACGTGAATCACAAGCCCAGCAACACCAAGGTGGACAAGAGAGTTGAGCCCAAATCT
TGTGACAAAACTCACACATGCCCACCGTGCCCAGCACCTGAACTCCTGGGGGGACC
GTCAGTCTTCCTCTTCCCCCCAAAACCCAAGGACACCCTCATGATCTCCCGGACCCC
TGAGGTCACATGCGTGGTGGTGGACGTGAGCCACGAAGACCCTGAGGTCAAGTTCA
ACTGGTACGTGGACGGCGTGGAGGTGCATAATGCCAAGACAAAGCCGCGGGAGGA
GCAGTACAACAGCACGTACCGTGTGGTCAGCGTCCTCACCGTCCTGCACCAGGACTG
GCTGAATGGCAAGGAGTACAAGTGCAAGGTCTCCAACAAAGCCCTCCCAGCCCCCA
TCGAGAAAACCATCTCCAAAGCCAAAGGGCAGCCCCGAGAACCACAGGTGTACACC
CTGCCCCCATCCCGGGAGGAGATGACCAAGAACCAGGTCAGCCTGACCTGCCTGGT
CAAAGGCTTCTATCCCAGCGACATCGCCGTGGAGTGGGAGAGCAATGGGCAGCCGG
AGAACAACTACAAGACCACGCCTCCCGTGCTGGACTCCGACGGCTCCTTCTTCCTCT
ATAGCAAGCTCACCGTGGACAAGAGCAGGTGGCAGCAGGGGAACGTCTTCTCATGC
TCCGTGATGCATGAGGCTCTGCACAACCACTACACGCAGAAGAGCCTCTCCCTGTCT
CCGGGTAAATGA (SEQ ID NO: 926)
Light chain
CAGGCTGTGCTGACTCAGCCGTCCTCAGTGTCCGTGTCCCCAGGACAGACAGCCAGC
ATCACCTGCTCTGGAGATAAATTGGGAGATAAATATGCTTCCTGGTATCAACAGAGG
CCAGGCCAGTCCCCTCTACTGGTCATTTATAAAGATTCCGAGCGGCCCTCAGGGATC
CCTGAACGATTCTCTGGCTCCAGCGCTGGGAATACAGCCACTCTGACCATCAGCGGG
ACCCAGCCTATGGATGAGGCTGACTATTACTGTCAGGCGTGGGACAGCGGTGCTGT
ATTCGGCGGAGGGACCAAGGTCACCGTCCTAGGTGGTCAGCCCAAGGCTGCCCCCT
CGGTCACTCTGTTCCCGCCCTCCTCTGAGGAGCTTCAAGCCAACAAGGCCACACTGG
TGTGTCTCATAAGTGACTTCTACCCGGGAGCCGTGACAGTGGCCTGGAAGGCAGAT
AGCAGCCCCGTCAAGGCGGGAGTGGAGACCACCACACCCTCCAAACAAAGCAACA
ACAAGTACGCGGCCAGCAGCTATCTGAGCCTGACGCCTGAGCAGTGGAAGTCCCAC
AGAAGCTACAGCTGCCAGGTCACGCATGAAGGGAGCACCGTGGAGAAGACAGTGG
CCCCTACAGAATGTTCATAG (SEQ ID NO: 927)
FL-G402-B2-133|B-000000400-000E
Heavy chain
CAGGTGCAGCTGGTGCAGTCTGGGGCAGAGGTGAAAAAGCCCGGGGAGTCTCTGAG
GATCTCCTGTAAGGCTTCTGGATACACCTTCACCGACTACTATCTGCACTGGGTGCG
ACAGGCCCCTGGAAAAGGGCTTGAGTGGATGGGATGGCTCAACCCTAACAGTGGTG
GCACAAACTATGCACAGAAGTTTCAGGGCAGGGTCACCATGACCAGGGACACGTCC
ATCAGCACAGCCTATATGGAGCTGAGCAGGCTGAGATCTGACGACACGGCCGTGTA
TTTTTGTGCGAGAGGGGCCTATGATTACGTCTGGGGGACTTCTCGCTATGTCGCTGA
TGCTTTCGATATCTGGGGCCAAGGGACAATGGTCACCGTCTCTTCAGCCTCCACCAA
GGGCCCATCGGTCTTCCCCCTGGCACCCTCCTCCAAGAGCACCTCTGGGGGCACAGC
GGCCCTGGGCTGCCTGGTCAAGGACTACTTCCCCGAACCGGTGACGGTGTCGTGGA
ACTCAGGCGCCCTGACAGCGGCGTGCACACCTTCCCGGCTGTCCTACAGTCCTCAGG
ACTCTACTCCCTCAGCAGCGTGGTGACAGTGCCCTCCAGCAGCTTGGGCACCCAGAC
CTACATCTGCAACGTGAATCACAAGCCCAGCAACACCAAGGTGGACAAGAGAGTTG
AGCCCAAATCTTGTGACAAAACTCACACATGCCCACCGTGCCCAGCACCTGAACTCC
TGGGGGGACCGTCAGTCTTCCTCTTCCCCCCAAAACCCAAGGACACCCTCATGATCT
CCCGGACCCCTGAGGTCACATGCGTGGTGGTGGACGTGAGCCACGAAGACCCTGAG
GTCAAGTTCAACTGGTACGTGGACGGCGTGGAGGTGCATAATGCCAAGACAAAGCC
GCGGGAGGAGCAGTACAACAGCACGTACCGTGTGGTCAGCGTCCTCACCGTCCTGC
ACCAGGACTGGCTGAATGGCAAGGAGTACAAGTGCAAGGTCTCCAACAAAGCCCTC
CCAGCCCCCATCGAGAAAACCATCTCCAAAGCCAAAGGGCAGCCCCGAGAACCACA
GGTGTACACCCTGCCCCCATCCCGGGAGGAGATGACCAAGAACCAGGTCAGCCTGA
CCTGCCTGGTCAAAGGCTTCTATCCCAGCGACATCGCCGTGGAGTGGGAGAGCAAT
GGGCAGCCGGAGAACAACTACAAGACCACGCCTCCCGTGCTGGACTCCGACGGCTC
CTTCTTCCTCTATAGCAAGCTCACCGTGGACAAGAGCAGGTGGCAGCAGGGGAACG
TCTTCTCATGCTCCGTGATGCATGAGGCTCTGCACAACCACTACACGCAGAAGAGCC
TCTCCCTGTCTCCGGGTAAATGA (SEQ ID NO: 928)
Light chain
CAGTCTGTGCTGACTCAGCCACCCTCAGCGTCTGGGACCCCCGGGCAGAGGGTCACC
ATCTCTTGTTCTGGAAGCAGCTCCAACATCGGGAGTAACACTGTAAACTGGTACCAG
CGACTCCCAGGAGCGGCCCCCCAACTCCTCATCTACAATAATGACCAGCGGCCCTCA
GGGATCCCTGACCGATTCTCTGGCTCCAAGTCTGGCACCTCAGGCTCCCTGGTCATC
AGTGGGCTCCAGTCTGAAGATGAGGCTGATTACTACTGTGCGTCATGGGATGACAGT
CTGAATGGTCGGGTGTTCGGCGGAGGGACCAAGCTGACCGTCCTAGGTGGTCAGCC
CAAGGCTGCCCCCTCGGTCACTCTGTTCCCGCCCTCCTCTGAGGAGCTTCAAGCCAA
CAAGGCCACACTGGTGTGTCTCATAAGTGACTTCTACCCGGGAGCCGTGACAGTGGC
CTGGAAGGCAGATAGCAGCCCCGTCAAGGCGGGAGTGGAGACCACCACACCCTCCA
AACAAAGCAACAACAAGTACGCGGCCAGCAGCTATCTGAGCCTGACGCCTGAGCAG
TGGAAGTCCCACAGAAGCTACAGCTGCCAGGTCACGCATGAAGGGAGCACCGTGGA
GAAGACAGTGGCCCCTACAGAATGTTCATAG (SEQ ID NO: 929)
FL-SKNAS-C2-384|B-000000401-000N
Heavy chain
GAAGTGCAGCTGGTGCAGTCTGGGGCTGAGGTGAAGAAGCCTGGGTCCTCGGTGAA
GGTCTCCTGCAAGGCTTCTGGAGGCACCCTCAGCAGTTCTGCTATCAGCTGGGTGCG
ACAGGCCCCTGGAGAAGGGCTTGAGTGGATGGGAGGGATCATCCCTATGTTTGATT
CAACAAACTACGCACAGAAGTTCCAGGGCAGAGTCACCATTACCGCGGACGAATCG
ACGAGCACAGCCTTCATGGAGCTGAGCAGCCTGAGATCTGACGACACGGCCGTGTA
TTATTGTGGGAGAGACCCCTACTATGATCGCCTTTCTGGGGGGGGGTACATGGACGT
CTGGGGCCAAGGAACCCTGGTCACCGTCTCGAGTGCCTCCACCAAGGGCCCATCGG
TCTTCCCCCTGGCACCCTCCTCCAAGAGCACCTCTGGGGGCACAGCGGCCCTGGGCT
GCCTGGTCAAGGACTACTTCCCCGAACCGGTGACGGTGTCGTGGAACTCAGGCGCC
CTGACAGCGGCGTGCACACCTTCCCGGCTGTCCTACAGTCCTCAGGACTCTACTCCC
TCAGCAGCGTGGTGACAGTGCCCTCCAGCAGCTTGGGCACCCAGACCTACATCTGCA
ACGTGAATCACAAGCCCAGCAACACCAAGGTGGACAAGAGAGTTGAGCCCAAATCT
TGTGACAAAACTCACACATGCCCACCGTGCCCAGCACCTGAACTCCTGGGGGGACC
GTCAGTCTTCCTCTTCCCCCCAAAACCCAAGGACACCCTCATGATCTCCCGGACCCC
TGAGGTCACATGCGTGGTGGTGGACGTGAGCCACGAAGACCCTGAGGTCAAGTTCA
ACTGGTACGTGGACGGCGTGGAGGTGCATAATGCCAAGACAAAGCCGCGGGAGGA
GCAGTACAACAGCACGTACCGTGTGGTCAGCGTCCTCACCGTCCTGCACCAGGACTG
GCTGAATGGCAAGGAGTACAAGTGCAAGGTCTCCAACAAAGCCCTCCCAGCCCCCA
TCGAGAAAACCATCTCCAAAGCCAAAGGGCAGCCCCGAGAACCACAGGTGTACACC
CTGCCCCCATCCCGGGAGGAGATGACCAAGAACCAGGTCAGCCTGACCTGCCTGGT
CAAAGGCTTCTATCCCAGCGACATCGCCGTGGAGTGGGAGAGCAATGGGCAGCCGG
AGAACAACTACAAGACCACGCCTCCCGTGCTGGACTCCGACGGCTCCTTCTTCCTCT
ATAGCAAGCTCACCGTGGACAAGAGCAGGTGGCAGCAGGGGAACGTCTTCTCATGC
TCCGTGATGCATGAGGCTCTGCACAACCACTACACGCAGAAGAGCCTCTCCCTGTCT
CCGGGTAAATGA (SEQ ID NO: 930)
Light chain
TCCTATGTGCTGACTCAGCCACCCTCGGTGTCCGGGGCCTTGAGACAGACCGCCACA
GTCACCTGCACTGGGAACAGCAGCAATGTTGGCAACCAAGGAGCAGCTTGGCTGAG
GCAGCACCAGGGCCGCCCTCCCAAACTTCTATCTTACAGGAATAACGACCGGCCCTC
AGGGGTCTCACAGAGATTTTCTGCTTCCACGTCAGGGAACACAGCCTCCCTGACCAT
TACTGGCCTCCAGTCTGAGGACGAGGCTGACTATTTCTGCTCAGCATGGGACAGCAG
CCGCAGTGGTTGGGTCTTCGGCGGAGGGACCAAGCTGACCGTCCTAGGTGGTCAGC
CCAAGGCTGCCCCCTCGGTCACTCTGTTCCCGCCCTCCTCTGAGGAGCTTCAAGCCA
ACAAGGCCACACTGGTGTGTCTCATAAGTGACTTCTACCCGGGAGCCGTGACAGTG
GCCTGGAAGGCAGATAGCAGCCCCGTCAAGGCGGGAGTGGAGACCACCACACCCTC
CAAACAAAGCAACAACAAGTACGCGGCCAGCAGCTATCTGAGCCTGACGCCTGAGC
AGTGGAAGTCCCACAGAAGCTACAGCTGCCAGGTCACGCATGAAGGGAGCACCGTG
GAGAAGACAGTGGCCCCTACAGAATGTTCATAG (SEQ ID NO: 931)
FL-SKNAS-C2-514|B-000000402-000X
Heavy chain
GAGGTCCAGCTGGTGCAGTCTGGAGCAGAGGTGAAAGAGCCGGGGGAGTCTCTGAG
GATCTCCTGTAAGGCTTCTGGATATGACTTTTCCAACTACTGGATCGCCTGGGTGCG
CCAGATGCCCGGGAAAGGCCTGGAGTGGATGGGGATCATCTATCCTGGTGACTCTG
ATACCAGATATAGCCCGTCCTTCCAAGGCCAGGTCACCATCTCAGTCGACAGGTCCA
TCAGCACCGCCTACCTGCAGTGGAGCAGCCTGAAGGCCTCGGACACCGCCCTGTATT
ACTGTTCCAGACCTACCTATTGGAGCACCACTTGGGGGAGGGAGTTTGACTACTGGG
GCCAAGGCACCCTGGTCACCGTCTCGAGTGCCTCCACCAAGGGCCCATCGGTCTTCC
CCCTGGCACCCTCCTCCAAGAGCACCTCTGGGGGCACAGCGGCCCTGGGCTGCCTGG
TCAAGGACTACTTCCCCGAACCGGTGACGGTGTCGTGGAACTCAGGCGCCCTGACA
GCGGCGTGCACACCTTCCCGGCTGTCCTACAGTCCTCAGGACTCTACTCCCTCAGCA
GCGTGGTGACAGTGCCCTCCAGCAGCTTGGGCACCCAGACCTACATCTGCAACGTG
AATCACAAGCCCAGCAACACCAAGGTGGACAAGAGAGTTGAGCCCAAATCTTGTGA
CAAAACTCACACATGCCCACCGTGCCCAGCACCTGAACTCCTGGGGGGACCGTCAG
TCTTCCTCTTCCCCCCAAAACCCAAGGACACCCTCATGATCTCCCGGACCCCTGAGG
TCACATGCGTGGTGGTGGACGTGAGCCACGAAGACCCTGAGGTCAAGTTCAACTGG
TACGTGGACGGCGTGGAGGTGCATAATGCCAAGACAAAGCCGCGGGAGGAGCAGT
ACAACAGCACGTACCGTGTGGTCAGCGTCCTCACCGTCCTGCACCAGGACTGGCTGA
ATGGCAAGGAGTACAAGTGCAAGGTCTCCAACAAAGCCCTCCCAGCCCCCATCGAG
AAAACCATCTCCAAAGCCAAAGGGCAGCCCCGAGAACCACAGGTGTACACCCTGCC
CCCATCCCGGGAGGAGATGACCAAGAACCAGGTCAGCCTGACCTGCCTGGTCAAAG
GCTTCTATCCCAGCGACATCGCCGTGGAGTGGGAGAGCAATGGGCAGCCGGAGAAC
AACTACAAGACCACGCCTCCCGTGCTGGACTCCGACGGCTCCTTCTTCCTCTATAGC
AAGCTCACCGTGGACAAGAGCAGGTGGCAGCAGGGGAACGTCTTCTCATGCTCCGT
GATGCATGAGGCTCTGCACAACCACTACACGCAGAAGAGCCTCTCCCTGTCTCCGGG
TAAATGA (SEQ ID NO: 932)
Light chain
TCCTATGAGCTGACTCAGCCACCCTCGGTGTCAGTGTCCCCAGGACAAACGGCCACG
ATCACCTGCTCTGGAGATGCATTGCCAAAAAAATATGCTTATTGGTACCAGCAGAAG
TCAGGCCAGGCCCCTGTGCTGGTCGTCTATGAGGACAGCAAACGACCCTCCGGGAT
CCCTGAGAGATTCTCTGGCTCCAGCTCAGGGACAATGGCCACCTTGACTATCAGTGG
GGCCCAGGTGGAGGATGAAGCTGACTACTACTGTTACTCAACAGACAGCAGTGGTA
ATCATTGGGTGTTCGGCGGAGGGACCAAGCTGACCGTCCTAGGTGGTCAGCCCAAG
GCTGCCCCCTCGGTCACTCTGTTCCCGCCCTCCTCTGAGGAGCTTCAAGCCAACAAG
GCCACACTGGTGTGTCTCATAAGTGACTTCTACCCGGGAGCCGTGACAGTGGCCTGG
AAGGCAGATAGCAGCCCCGTCAAGGCGGGAGTGGAGACCACCACACCCTCCAAACA
AAGCAACAACAAGTACGCGGCCAGCAGCTATCTGAGCCTGACGCCTGAGCAGTGGA
AGTCCCACAGAAGCTACAGCTGCCAGGTCACGCATGAAGGGAGCACCGTGGAGAAG
ACAGTGGCCCCTACAGAATGTTCATAG (SEQ ID NO:933)
A673-C2-110|B-000000403-000F
Heavy chain
CAGGTCCAGCTGGTGCAGTCTGGGACTGAGGTGAAGAAGCCTGGGGCCTCAGTGAG
GGTCTCCTGTAAGCCATCTGCAGACACCTTCACCAGCTACTATGTGCACTGGGTGCG
ACAGGCCCCTGGACAAGGGCTTGAGTGGATGGGCACAATCAACCCCACCGGAACTT
ACACAAGGTACGCACAGCAGTTCCAGGGCAGAGTCACCGTGACCAGGGACACGTCC
ACGAGCTCAGTGTACATGGAATTGAGCAGCCTGAGATCTGAGGACTCGGCCGTGTA
TTATTGTGCGAGAGACGTAAGACCCTATACAGCTATGTACTCCTTTGACAACTGGGG
GCGGGGGACCACGGTCACCGTCTCGAGTGCCTCCACCAAGGGCCCATCGGTCTTCCC
CCTGGCACCCTCCTCCAAGAGCACCTCTGGGGGCACAGCGGCCCTGGGCTGCCTGGT
CAAGGACTACTTCCCCGAACCGGTGACGGTGTCGTGGAACTCAGGCGCCCTGACAG
CGGCGTGCACACCTTCCCGGCTGTCCTACAGTCCTCAGGACTCTACTCCCTCAGCAG
CGTGGTGACAGTGCCCTCCAGCAGCTTGGGCACCCAGACCTACATCTGCAACGTGA
ATCACAAGCCCAGCAACACCAAGGTGGACAAGAGAGTTGAGCCCAAATCTTGTGAC
AAAACTCACACATGCCCACCGTGCCCAGCACCTGAACTCCTGGGGGGACCGTCAGT
CTTCCTCTTCCCCCCAAAACCCAAGGACACCCTCATGATCTCCCGGACCCCTGAGGT
CACATGCGTGGTGGTGGACGTGAGCCACGAAGACCCTGAGGTCAAGTTCAACTGGT
ACGTGGACGGCGTGGAGGTGCATAATGCCAAGACAAAGCCGCGGGAGGAGCAGTA
CAACAGCACGTACCGTGTGGTCAGCGTCCTCACCGTCCTGCACCAGGACTGGCTGAA
TGGCAAGGAGTACAAGTGCAAGGTCTCCAACAAAGCCCTCCCAGCCCCCATCGAGA
AAACCATCTCCAAAGCCAAAGGGCAGCCCCGAGAACCACAGGTGTACACCCTGCCC
CCATCCCGGGAGGAGATGACCAAGAACCAGGTCAGCCTGACCTGCCTGGTCAAAGG
CTTCTATCCCAGCGACATCGCCGTGGAGTGGGAGAGCAATGGGCAGCCGGAGAACA
ACTACAAGACCACGCCTCCCGTGCTGGACTCCGACGGCTCCTTCTTCCTCTATAGCA
AGCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGGGT
AAATGA (SEQ ID NO: 934)
Light chain
TCCTATGTGCTGACTCAGCCACCCTCGGTGTCAGTGTCCCCAGGACAGACGGCCACG
ATCACCTGCTCTGGAGATGCATTGCCAAAGCAATATGCTAATTGGTACCAGCAGAA
ACCAGGCCAGGCCCCTGTGTTAATGATATATAAAGACAGTGACAGGCCCTCAGGGA
TCCCTGAGCGATTCTCTGGCTCCAGCTCAGGGACAAAAGCCACGTTGACCATCAGTG
GAGTCCAGGCAGAAGACGAGGCTGACTATTACTGTCAATCAGCAGACAACAGTGGT
ACTAATTATGTCTTCGGAACTGGGACCAAGCTGACCGTCCTAGGTGGTCAGCCCAAG
GCTGCCCCCTCGGTCACTCTGTTCCCGCCCTCCTCTGAGGAGCTTCAAGCCAACAAG
GCCACACTGGTGTGTCTCATAAGTGACTTCTACCCGGGAGCCGTGACAGTGGCCTGG
AAGGCAGATAGCAGCCCCGTCAAGGCGGGAGTGGAGACCACCACACCCTCCAAACA
AAGCAACAACAAGTACGCGGCCAGCAGCTATCTGAGCCTGACGCCTGAGCAGTGGA
AGTCCCACAGAAGCTACAGCTGCCAGGTCACGCATGAAGGGAGCACCGTGGAGAAG
ACAGTGGCCCCTACAGAATGTTCATAG (SEQ ID NO: 935)
C. CDR (Aminoacid sequence)
C.1-Binding to NRR

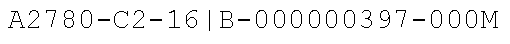

HCDR1 aa:
Start 26 End 33
GGTFSSFA (SEQ ID NO: 936)
HCDR2
Start 51 End 60
IIPILGRTNY (SEQ ID NO: 937)
HCDR3
Start 97 End 112|
ARDREYSLSLGGSDYW (SEQ ID NO: 938)
LCDR1
Start 26 End 34
SSNIGAGYD (SEQ ID NO: 241)
LCDR2
Start 52 End 55
ANTN (SEQ ID NO: 939)
LCDR3
Start 91 End 101|
QSYDSSLSGRV (SEQ ID NO: 940)

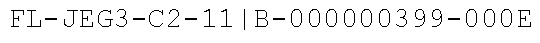

HCDR1 aa:
Start 28 End 35
GFSFDNY (SEQ ID NO: 941)
HCDR2
Start 53 End 62
ISGSGGQIFY (SEQ ID NO: 942)
HCDR3
Start 99 End 115|
ARDFYRTPGYNTNWGYW (SEQ ID NO: 943)
LCDR1
Start 26 End 31
GLGNKY (SEQ ID NO: 944)
LCDR2
Start 49 End 52
QDSK (SEQ ID NO: 945)
LCDR3
Start 88 End 96|
QAWDRPAVI (SEQ ID NO: 946)
C.2-Additional Notch Antibodies

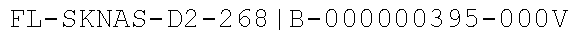

HCDR1 aa:
Start 26 End 33
HCDR2
Start 51 End 60
ISGSGGSTYY (SEQ ID NO: 947)
HCDR3
Start 97 End 109|
ARHGHSSGYFVYW (SEQ ID NO: 948)
LCDR1
Start 26 End 34
SSNIGAPYD (SEQ ID NO: 949)
LCDR2
Start 52 End 55
GNTN (SEQ ID NO: 950)
LCDR3
Start 91 End 101|
QSYDHSLSGWV (SEQ ID NO: 951)

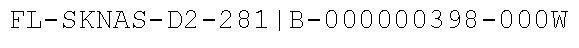

HCDR1
Start 26 End 33
HCDR2
Start 51 End 60
ISGSGGSTYY (SEQ ID NO: 952)
HCDR3
Start 97 End 114|
ARSQLPYCGGDCYGSDYW (SEQ ID NO: 953)
LCDR1
Start 26 End 31
KLGDKY (SEQ ID NO: 880)
LCDR2
Start 49 End 52
KDSE (SEQ ID NO: 954)
LCDR3
Start 88 End 95|
QAWDSGAV (SEQ ID NO: 955)

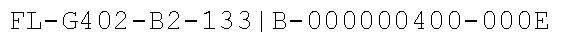

HCDR1
Start 26 End 33
GYTFTDYY (SEQ ID NO: 267)
HCDR2
Start 51 End 60
LNPNSGGTNY (SEQ ID NO: 956)
HCDR3
Start 97 End 118|
ARGYDYVWGTSRYVADAFDIW (SEQ ID NO: 957)
LCDR1
Start 26 End 33
SSNIGSNT (SEQ ID NO: 226)
LCDR2
Start 51 End 54
NNDQ (SEQ ID NO: 958)
LCDR3
Start 90 End 100|
ASWDDSLNGRV (SEQ ID NO: 303)

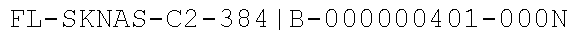

HCDR1
Start 26 End 33
GGTLSSSA (SEQ ID NO: 959)
HCDR2
Start 51 End 60
IIPMFDSTNY (SEQ ID NO: 960)
HCDR3
Start 97 End 114|
GRDPYYDRLSGGGYMDVW (SEQ ID NO: 961)
LCDR1
Start 26 End 33
SSNVGNQG (SEQ ID NO: 962)
LCDR2
Start 51 End 54
RNND (SEQ ID NO: 963)
LCDR3
Start 90 End 100|
SAWDSSRSGWV (SEQ ID NO: 964)

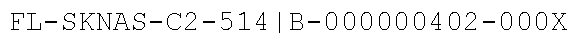

HCDR1
Start 26 End 33
GYDFSNYW (SEQ ID NO: 965)
HCDR2
Start 51 End 60
IYPGDSDTRY (SEQ ID NO: 966)
HCDR3
Start 97 End 113|
SRPTYWSTTWGREFDYW (SEQ ID NO: 967)
LCDR1
Start 26 End 31
ALPKKY (SEQ ID NO: 968)
LCDR2
Start 49 End 52
EDSK (SEQ ID NO: 969)
LCDR3
Start 88 End 98|
YSTDSSGNHWV (SEQ ID NO: 970)

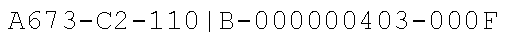

HCDR1
Start 26 End 33
ADTFTSYY (SEQ ID NO: 971)
HCDR2
Start 51 End 60
INPTGTYTRY (SEQ ID NO: 972)
HCDR3
Start 97 End 112|
ARDVRPYTAMYSFDNW (SEQ ID NO: 973)
LCDR1
Start 26 End 31
ALPKQY (SEQ ID NO: 974)
LCDR2
Start 49 End 52
KDSD (SEQ ID NO: 975)
LCDR3
Start 88 End 98|
QSADNSGTNYV (SEQ ID NO: 976)

What is claimed is:

1. An isolated or purified Notch1-specific antibody molecule or an antigen binding portion thereof comprising at least one light chain sequence comprising a sequence of amino acids selected from the group consisting of SEQ ID NOS. 23-42 or 46-48 and at least one heavy chain sequence comprising a sequence of amino acids selected from the group consisting of SEQ ID NOS. 3-22 or 43-45, or at least one light chain comprising an amino acid sequence having at least 80% identity with the sequence set forth in any one or more of SEQ ID NOS. 23-42 or 46-48, and wherein said heavy chain comprises an amino acid sequence or at least one heavy chain comprising an amino acid sequence having at least 80% identity with one of SEQ ID NOS. SEQ ID NOS. 3-22 or 43-45.

2. The antigen-binding portion according to claim 1, wherein said portion is selected from the group consisting of: a Fab fragment, an F(ab′)₂ fragment and an Fv fragment.

3. An isolated or purified Notch1-specific antibody molecule comprising a heavy chain variable region comprising the amino acid sequence as set forth in one of SEQ ID NOs. 3-22 or 43-45 or a glycosylation variant, fusion molecule or a chemical derivative thereof or an antigen-binding region thereof that specifically binds Notch1.

4. The antibody of claim 1 or 3 that comprises a mutant immunoglobulin chain, the mutant antibody having higher affinity for an antigen than a parent antibody that comprises a parent immunoglobulin chain, wherein the mutant immunoglobulin chain comprises an amino acid substitution that eliminates a variable region glycosylation site of the parent immunoglobulin chain, said elimination having the effect of increasing the affinity of the mutant antibody relative to the parent antibody.

5. A cell line that produces the antibody as set forth in claim 1.

6. A method for diagnosing an oncogenic disorder associated with expression of Notch1 or determining the prognosis for developing an oncogenic disorder associated with expression of Notch1 in a subject comprising contacting a sample from the subject with the monoclonal antibody of claim 1, and detecting the binding of the monoclonal antibody with the sample, wherein binding of the monoclonal antibody to the sample is indicative of the diagnosis of said neoplasia.

7. The method according to claim 6, wherein said antibody is labeled.

8. A method of detecting the presence or location of an Notch1-expressing tumor in a subject, comprising the steps of: a) administering the antibody according to claim 1 to the subject; and b) detecting binding of said antibody, wherein said binding indicates the presence or location of the tumor.

9. A method for determining the prognosis of the course of a malignant disease associated with expression of Notch1, comprising obtaining a sample from a subject suspected of containing tumor cells, contacting said sample with the antibody of claim 1 or an antigen-binding fragment thereof, wherein binding of the antibody or the antigen-binding fragment thereof with tumor cells in the sample is indicative of a tumor and gives a prognoses for the course of a malignant disease in said subject.

10. A method for selecting a therapy for a patient or a patient population with a tumor associated with or mediated by expression of Notch1 comprising: (a) determining whether the patient's tumor is known to over express Notch1 bearing cells relative to normal and (b) selecting an Notch1 inhibitory agent as the therapy if the patient's tumor is known to over express said Notch1.

11. The method of claim 10, wherein the agent is: (i) the isolated antibody or antigen-binding fragment thereof according to claim 1.

12. A method for following progress of a therapeutic regime designed to alleviate an oncogenic disorder associated with or characterized by expression of Notch1 comprising:
(a) assaying a biological sample from a subject to determine level of Notch1 at a first time point by contacting said sample with the antibody according to claim 1;
(b) assaying level of Notch1 at a second time point; and
(c) comparing said level at said second time point to the level determined in (a) as a determination of effect of said therapeutic regime.

13. A method for determining the expression of Notch1 (a) in a test tissue sample suspected of containing said polypeptide and (b) a control normal tissue sample of the same tissue type, said method comprising exposing the test and control tissue samples to the anti-Notch1 antibody of claim 1 and determining the relative binding of said antibody to said polypeptide in each of said samples.

14. The method according to claim 13, further comprising quantifying the level of Notch1 expression in said control sample to obtain a normal or control value and comparing the same to the level obtained in the test tissue sample to determine the overall expression of Notch1 in said test tissue sample.

15. A method for determining the prognosis for survival for a patient presenting with a cancer mediated by Notch1, comprising: (a) measuring a level of Notch1 receptor polypeptide in a cancer cell-containing sample from said patient, and (b) comparing the level of Notch1 receptor polypeptide in said sample to a reference level of Notch1 polypeptide from normal tissue, wherein a lower level of Notch1 polypeptide relative to said reference level correlates with increased survival of said patient, wherein step (a) using the antibody of claim 1.

16. A method for prognostic evaluation of a patient suspected of exhibiting an oncogenic disorder associated with expression of Notch1 comprising: (a) determining the concentration of Notch1 present in a biological sample, taken from the patient, suspected of containing oncogenic tissue; (b) comparing the level determined in step (a) to the concentration range of Notch1 polypeptide known to be present in normal, non-oncogenic tissue of the same type as present in the biological sample; and (c) evaluating the prognosis of said patient based on the comparison in step (b), wherein a high level of Notch1 expression in step (a) indicates an aggressive form of cancer and therefore a poor prognosis., wherein step (a) comprises contacting said biological sample with the antibody of claim 1.

17. The method according to claim 16 further comprising a step prior to step (a) comprising purifying said Notch1 polypeptide from the biological sample.

18. The method of claim 17 wherein the purifying method is immunoaffinity chromatography.

19. A method for determining the prognosis of an individual with an oncogenic disorder or a susceptibility to a pathological hyperproliferative disorder associated with expression of Notch1 in a subject, comprising: a) determining the expression levels of Notch1 in a biological sample collected from said patient in different states of the individual; and b) comparing the expression profile of Notch1 in the different states, wherein a higher level of the expression in a later state sample compared with an early state sample indicates a poor prognosis, wherein step (a) comprises contacting said biological sample with the antibody of claim 1.

20. A method of detecting a pathological hyperproliferative oncogenic disorder associated with expression of Notch1 in a subject comprising: a) determining the level of expression of Notch1 in a first tissue sample obtained from said first individual; and b) comparing said level obtained in step (a) with that of a normal tissue sample obtained from said first individual or a second unaffected individual; wherein a difference in said expression of Notch1 is an indication that the first individual may present have said pathological hyperproliferative oncogenic disorder, wherein step (a) comprises contacting said biological sample with the antibody of claim 1.

21. The method according to claim 20, wherein said difference is an increase in the expression level of Notch1 relative to the normal tissue.

22. A method for determining onset, progression, or regression, of an oncogenic disorder associated with expression of Notch1 in a subject, comprising:
(i) (a) obtaining from a subject a first biological sample,
(b) contacting the first sample with a therapeutically effective amount of a therapeutic anti-Notch1 antibody sufficient to down regulate Notch1 expression, wherein said antibody is other than the antibody of claim 1;
(c) determining specific binding between the antibody in the first sample and Notch1 bearing cells,
(ii) (a) obtaining subsequently from the subject a second biological sample,
(b) contacting the second biological sample with the antibody of claim 1,
(c) determining specific binding between the antibody in the second sample and Notch1 bearing cells, and
(iii) comparing the determination of binding in the first sample to the determination of specific binding in the second sample as a determination of the onset, progression, or regression of the neoplasia.

23. A method for monitoring the efficacy of an antibody in correcting an abnormal level of Notch1 in a subject presenting with an oncogenic disorder associated with increased of Notch1, comprising
i) administering an effective amount of a conventional Notch1 antibody other than the antibody of claim 1 to said subject; and
ii) determining a level of Notch1 in said subject following the administration of the conventional antibody, wherein a change in the level of Notch1 towards a normal level is indicative of the efficacy of said antibody.

24. The method according to claim 23 wherein step (ii) comprises contacting a tissue sample obtained from said subject with the antibody according to claim 1 under conditions favoring the formation of a complex between Notch1 expressing cells and said antibody and detecting said complex as a determination of the expression level of Notch1 in said sample.

25. The method as claimed in claim 13, wherein said antibody comprises a detectable label.

26. The method as claimed in claim 25, wherein said detectable label is selected from the group consisting of fluorescein, rhodamine, phycoerythrin, biotin, and strepavidin.

27. The method as claimed in claim 26, wherein said antibody is detected by a method selected from the group consisting of flow cytometric analysis, immunochemical detection and immunoblot analysis.

28. The method of claim 27, wherein said antibody or fragment is in solution.

29. The method as claimed in claim 28, wherein said biological sample comprises soft tissue tumor cells and non-malignant cells.

30. An article of manufacture, comprising: a container; a label on the container; and a composition comprising an active agent contained within the container, wherein the composition is effective for detecting Notch1 in neoplastic tissue or dysplastic cells and wherein the label on the container indicates that the composition is effective for diagnosing conditions associated with expression of Notch1 polypeptide in said neoplastic tissue compared to normal tissue.

31. The article of manufacture according to claim 30, wherein said active ingredient comprises the antibody according to claim 1.

32. An in vivo method of imaging an oncogenic disorder associated with expression of Notch1 comprising the steps of:
(a) administering to a subject an imaging-effective amount of the labeled monoclonal antibody according to claim 1 or fragment thereof and a pharmaceutically effective carrier; and
(b) detecting the binding of said labeled monoclonal antibody or fragment thereof to Notch1 expressing cells associated with said disorder.

33. The method of claim 32, wherein said monoclonal antibody or fragment thereof is radiolabeled.

34. The method of claim 33, wherein said detecting involves radioactive imaging.

35. A method for determining whether a cancer is susceptible to treatment with an anti-neoplastic agent comprising the steps of: (a) obtaining a sample of the cancer, (b) measuring the level of Notch1 in the sample, (c) comparing the level with a predetermined value, and (d) determining that, if the measured level is larger than the predetermined value, the cancer is susceptible to treatment with the anti-neoplastic agent, wherein step (a) comprises contacting said biological sample with the antibody of claim 1.

36. A pharmaceutical composition for in vivo imaging of an oncogenic disorder associated with expression of Notch1 comprising the monoclonal antibody of claim 1 or an antigen binding fragment thereof which is labeled and which binds Notch1 in vivo; and a pharmaceutically acceptable carrier.

37. A method for detecting Notch1 or one of its isoforms or a fragment thereof in a biological sample, comprising: (a) contacting said biological sample the antibody of claim 1 thereby forming an antibody-polypeptide complex; and (b) detecting said antibody-polypeptide complex as indicating presence of said Notch1 in said sample.

38. The method according to claim 37, wherein said antibody is detectably labeled.

39. The method of claim 37 in which step (b) comprises an immunoassay, wherein said immunoassay is selected from the group comprising: direct, indirect, capture, competitive binding, and displacement.

40. The method of claim 37 in which said step of detecting the presence of Notch1 comprises a qualitative analysis.

41. The method of claim 37 in which said step of detecting the presence of Notch1 comprises a quantitative analysis.

42. The method of claim 39 in which said binding assay comprises a clinical diagnostic assay.

43. The method of claim 42 which is of the type selected from the group consisting of: IFA, linear flow, radial flow, Western Blot, ELISA, dip stick, EIA, fluorescent polarization, enzyme capture, and RIA.

44. A method for diagnosing an oncogenic disorder associated with expression of Notch1 comprising: a) measuring by radioimmunoassay, competitive-binding assay, Western blot analysis, ELISA assay, or sandwich assay the amount of Notch1 protein in a sample obtained from a patient, using an antibody that specifically binds to Notch1; and b) comparing the amount of antibody bound to said Notch1 protein to a normal control tissue sample, wherein increased expression or over-expression of Notch1 in the sample obtained from the patient relative to the normal control tissue sample is diagnostic of an oncogenic disorder associated with expression of Notch1, wherein said antibody is as described in claim 1.

45. The method of claim 44, wherein said sample obtained from a patient is tissue biopsy.

46. A diagnostic or monitoring method comprising: a) obtaining a sample of tissue from an individual in need of diagnosis or monitoring for cancer; b) detecting levels of Notch1 protein in said sample, c) scoring said sample for Notch1 protein levels; and d) comparing said scoring to that obtained from a control tissue sample to determine the prognosis associated with said cancer, wherein step (b) comprises contacting said tissue sample with the antibody of claim 1.

47. The diagnostic or monitoring method according to claim 46, wherein said scoring comprises using a scale of 0 to 4, where 0 is negative (no detectable Notch1 or level of Notch1 comparable to a control level), and 4 is high intensity staining in the majority of cells and wherein a score of 1 to 4 indicates a poor prognosis while a score of 0 indicates a good prognosis.

48. The diagnostic or monitoring method according to claim 47, wherein the detecting or measuring step is selected from the group of methods consisting of immunoblotting, immunohistochemistry and immunocytochemistry.

49. The diagnostic or monitoring method according to claim 46 wherein the step b) is done by Fluorescence-Activated Cell Sorting (FACS).

50. A method for determining a chemotherapeutic regimen comprising an Notch1 targeted agent, for treating a tumor in a patient comprising: (a) obtaining a tissue sample of the tumor; (b) detecting levels of Notch1 levels in said sample, (c) scoring said sample for expression of Notch1 levels, (d) repeating steps (b)-(c) in a matching non-malignant tissue sample to obtain a threshold level (e) determining a chemotherapeutic regimen by comparing the differential Notch1 expression level of step (c) and the threshold level of step (d), wherein an increase in differential Notch1 expression level in step (c) relative to step (d) dictate placing said patient in the chemotherapeutic regimen wherein step (b) comprises contacting said tissue sample with the antibody of claim 1.

51. The method according to claim 50, wherein said scoring comprises using a scale of 0 to 4, where 0 is negative (no detectable Notch1 or level of Notch1 comparable to a control level), and 4 is high intensity staining in the majority of cells and wherein a score of 1 to 4 (i.e. a positive score) indicates chemotherapeutic regimen.

52. A method for predicting disease-free survival and overall survival in a patient with an oncogenic disorder associated with Notch1 expression comprising: a) obtaining a sample of diseased or cancerous tissue from an individual presenting with said oncogenic disorder, b) detecting levels of Notch1 expressing cells in said cancer cells or cancer tissue of said sample; c) scoring said samples for expression of Notch1 levels; and d) comparing said scoring to that obtained from a control sample to determine likelihood of disease-free survival and overall survival associated with Notch1, wherein step (b) comprises contacting said tissue sample with the antibody of claim 1.

53. The method according to claim 52, wherein said scoring comprises using a scale of 0 to 4, where 0 is negative (no detectable Notch1 or level of Notch1 comparable to a control level), and 4 is high intensity staining in the majority of cells and wherein a score of 1 to 4 (i.e. a positive score) indicates a poor prognosis for disease free and overall survival in patients with said disorder.

54. A method for treating an Notch1 mediated cancer comprising: a) obtaining a sample of diseased tissue from a patient in need of treatment of said cancer; b) determining the level of expression of Notch1 levels in said tissue sample; c) scoring said samples for expression of Notch1 levels; d) correlating said score to identify patients likely to benefit from treatment with an Notch1 antagonist, wherein said step of correlating comprises comparing said scoring to that obtained from a control sample, e) treating said patient with a therapeutic regime known to improve the prognosis for said cancer; f) repeating steps “a” and “b”, and g) adjusting the therapeutic regime known to improve the prognosis for said cancer; h) repeating steps a-f as frequently as deemed appropriate, wherein step (b) comprises contacting said tissue sample with the antibody of claim 1.

55. The method according to claim 54, wherein said scoring comprises using a scale of 0 to 4, where 0 is negative (no detectable Notch1 or level of Notch1 comparable to a control level), and 4 is high intensity staining in the majority of cells.

56. A method of treating a Notch1 mediated disorder comprising administrating to a patient in need thereof the antibody of claim 1 sufficient to treat said disorder, wherein said antibody is an agonist antibody specific for Notch1.

57. A method of treating a Notch1 mediated disorder comprising administrating to a patient in need thereof the antibody of claim 1 sufficient to treat said disorder, wherein said antibody is an antagonist agonist antibody specific for Notch1.

58. A method for determining the effect of a therapeutic regimen for alleviating an Notch1 mediated disorder, wherein said regimen comprises the use of an Notch1 antagonist, the method comprising the steps of: a) obtaining a cell or tissue sample from an individual undergoing said therapeutic regimen b) measuring the levels of Notch1 in said cell or tissue sample; c) scoring said sample for Notch1 protein levels, and d) comparing said levels to that of a control sample to predict the responsiveness of said Notch1 mediated disorder to said therapeutic regimen, wherein step (b) comprises contacting said tissue sample with the antibody of claim 1.

59. A method for stratifying a patient presenting with an oncogenic disorder mediated by Notch1 for a clinical trial comprising: a) obtaining a tissue sample from said patient, b) detecting levels of Notch1 protein in said sample, c) scoring said sample for Notch1 protein levels; and d) stratifying said patient for said clinical trial based on the results of the scoring step, wherein step (b) comprises contacting said tissue sample with the antibody of claim 1.

60. The method according to claim 59, wherein said scoring comprises using a scale of 0 to 4, where 0 is negative (no detectable Notch1 or level of Notch1 comparable to a control level), and 4 is high intensity staining in the majority of cells.

61. A method of classifying or staging a breast tumor characterized by expression of Notch1 comprising the steps of: i) providing a breast tumor sample, ii) detecting expression Notch1 in the sample, iii) scoring the sample for Notch1 expression level, and iv) classifying the tumor as belonging to a tumor subclass based on the results of the scoring step, wherein step ii) comprises contacting said tissue sample with the antibody of claim 1.

62. The method according to claim 61, wherein said scoring comprises using a scale of 0 to 4, where 0 is negative (no detectable Notch1 or level of Notch1 comparable to a control level), and 4 is high intensity staining in the majority of cells.

63. A method of treating a human tumor susceptible to an antibody induced cellular cytotoxicity in a mammal, wherein said human tumor expresses an antigen which specifically binds to the monoclonal antibody which has the cellular cytotoxicity inducing characteristics of said antibody of claim 1 or a cellular cytotoxicity inducing antigen binding fragment thereof, comprising administering to said mammal said antibody or said antigen binding fragment thereof in an amount effective to induce cellular cytotoxicity and thereby reduce said mammal's tumor burden.

64. The method of claim 63 wherein said antibody is conjugated to a cytotoxic moiety.

65. The method of claim 63, wherein said cytotoxic moiety is a radioactive isotope.

66. The method of claim 63 wherein said antibody activates complement.

67. The method of claim 63 wherein said antibody mediates antibody dependent cellular cytotoxicity.

68. The isolated antibody or antigen binding fragment of any one of claim 1 or 3 conjugated with a member selected from the group consisting of cytotoxic moieties, enzymes, radioactive compounds, and hematogenous cells.

69. The isolated antibody according to claim 1 wherein said antibody is a multivalent antibody.

70. The antibody according to claim 69, wherein said antibody is a bispecific, tetravalent antibody specific for Notch1.

71. The isolated antibody of claim 1 which comprises an antigen binding region and a variant Fe region, wherein said variant Fc region: (A) differs from a wild-type Fe region by comprising an amino acid modification and (B) binds an FcγR with an increased affinity relative to a said wild-type Fc region.

72. The antibody according to claim 71 wherein said FcγR is FcγRIIIA.

73. The antibody according to claim 72, wherein said variant Fc region of said antibody has decreased affinity for FcγRIIB relative to said wild-type Fc region.

74. A variant antibody derived from the antibody of claim 1, wherein said antibody comprises an Fc region, said variant mediates antibody-dependent cell-mediated cytotoxicity (ADCC) in the presence of human effector cells more effectively, or binds an Fc gamma receptor (FcγR) with better affinity, than the parent polypeptide and comprises at least one amino acid modification in the Fc region.

75. A method of treating a human tumor susceptible to antibody induced cellular cytotoxicity in a mammal, wherein said human tumor expresses a Notch1 receptor which specifically binds to a monoclonal antibody which has the cellular cytotoxicity inducing characteristics of the antibody of claim 1, comprising administering to said mammal said monoclonal antibody or said antigen binding fragment thereof in an amount effective to induce cellular cytotoxicity and thereby reduce said mammal's tumor burden.

76. The method of claim 75 wherein said monoclonal antibody is conjugated to a cytotoxic moiety.

77. The method of claim 76 wherein said cytotoxic moiety is a radioactive isotope.

78. The method of claim 77 wherein said monoclonal antibody activates complement.

79. The method of claim 78 wherein said monoclonal antibody mediates antibody dependent cellular cytotoxicity.

80. The isolated antibody or antigen binding fragments of claim 1 conjugated with a member selected from the group consisting of cytotoxic moieties, enzymes and radioactive compounds.

81. A Notch1 antibody variant derived from the antibody of claim 1 comprising an Fc region, which variant mediates antibody-dependent cell-mediated cytotoxicity (ADCC) in the presence of human effector cells more effectively, or binds an Fc gamma receptor (FcγR) with better affinity, than the parent polypeptide and comprises at least one amino acid modification in the Fc region.

82. A Notch1 antibody or variant thereof derived from the antibody of claim 1 or 3 which recognizes mutations in the negative regulatory region (NRR), wherein said mutations within said NRR are as set forth in FIG. 12A.

83. The antibody according to claim 82, wherein said antibody is WC-12.
